(19) United States
(12) Patent Application Publication
(10) Pub. No.: US 2019/0330335 A1
(43) Pub. Date: Oct. 31, 2019

(54) ANTI-TREM2 ANTIBODIES AND METHODS OF USE THEREOF

(71) Applicant: ALECTOR LLC, South San Francisco, CA (US)

(72) Inventors: Tina SCHWABE, San Francisco, CA (US); Francesca AVOGADRI-CONNORS, South San Francisco, CA (US); Helen LAM, South San Francisco, CA (US); Ilaria TASSI, San Francisco, CA (US); Seung-Joo LEE, Benicia, CA (US); Arnon ROSENTHAL, Woodside, CA (US)

(73) Assignee: ALECTOR LLC, South San Francisco, CA (US)

(21) Appl. No.: 15/766,363

(22) PCT Filed: Oct. 6, 2016
(86) PCT No.: PCT/US2016/055828

§ 371 Date: Apr. 5, 2018

Related U.S. Application Data
(60) Provisional application No. 62/369,666, filed on Aug. 1, 2016.
(60) Provisional application No. 62/238,044, filed on Oct. 6, 2015.

(51) Int. Cl. C07K 16/28; A61P 25/28

(52) U.S. Cl. CPC C07K 16/2803; A61P 25/28; C07K 2317/34; A61K 2039/505; C07K 2317/75; C07K 2317/24; C07K 2317/76; C07K 2317/92

(57) ABSTRACT

The present disclosure is generally directed to compositions that include antibodies, e.g., monoclonal, chimeric, humanized antibodies, antibody fragments, etc., that specifically bind a TREM2 protein, e.g., a mammalian TREM2 or human TREM2, and use of such compositions in preventing, reducing risk, or treating an individual in need thereof.

### CROSS-REFERENCE TO RELATED APPLICATIONS

This application is a U.S. national stage application of PCT/US2016/055828, filed internationally on Oct. 6, 2016, which claims the benefit of U.S. Provisional Application No. 62/238,044, filed Oct. 6, 2015, and U.S. Provisional Application No. 62/369,666, filed Aug. 1, 2016, each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 735022000900SEQLIST.txt, date recorded: Apr. 5, 2018, size: 651 KB).

### FIELD OF THE PRESENT DISCLOSURE

The present disclosure relates to anti-TREM2 antibodies and therapeutic uses of such antibodies.

### BACKGROUND OF THE PRESENT DISCLOSURE

Triggering receptor expressed on myeloid cells-2 (TREM2) is an immunoglobulin-like receptor that is expressed primarily on myeloid lineage cells, such as macrophages, dendritic cells, monocytes, Langerhans cells of skin, Kupffer cells, osteoclasts, and microglia; and is required for modulation of Toll-like receptor (TLR) signaling, the modulation of inflammatory cytokines, as well as for normal osteoclast development. TREM2 was discovered as a member of the TREM transmembrane glycoproteins, which belong to the single immunoglobulin variable (IgV) domain receptor family. The genes encoding human and mouse TREMs map to human chromosome 6p21.1 and mouse chromosome 17C3, respectively. The TREM cluster includes genes encoding TREM1, TREM2, TREM4, and TREM5, as well as the TREM-like genes in both human and mouse. Additionally TREM3 and plasmocytoid dendritic cell (pDC)-TREM were identified in mouse. The TREM-like genes, TREML1 and TREML2 in humans, and Treml1 and Treml2 in mouse, encode TLT-1 and TLT-2 proteins respectively. The two best characterized of these receptor family, TREM1 and TREM2, display −20% sequence homology as well as some homology with other members of the Ig-SF such as activating NK cells receptors (20% identity with NKp44) and act through association with a DAP12-mediated pathway for signaling.

TREM2 was originally cloned as a cDNA encoding a TREM1 homologue (Bouchon, A et al., J Exp Med, 2001. 194(8): p. 1111-22). This receptor is a glycoprotein of about 40 kDa, which is reduced to 26 kDa after N-deglycosylation. The TREM2 gene encodes a 230 amino acid-length protein that includes an extracellular domain, a transmembrane region and a short cytoplasmic tail. The extracellular region, encoded by exon 2, is composed of a single type V Ig-SF domain, containing three potential N-glycosylation sites. The putative transmembrane region contains a charged lysine residue. The cytoplasmic tail of TREM2 lacks signaling motifs and is thought to signal through the signaling adaptor molecule DAP12/TRYROBP.

The signaling adaptor molecule DAP12 is expressed as a homodimer at the surface of a variety of cells participating in innate immune response, including microglia, macrophages, granulocytes, NK cells, and dendritic cells (DC). DAP12 is a member of the type I transmembrane adapter protein family on the basis of homology with the human T-cell receptor (TCR)-associated CD3 chains and the Fc receptor (FcR) γ-chain (Turnbull, I R and Colonna, M, Nat Rev Immunol, 2007. 7(2): p. 155-61). These proteins share many structural and functional characteristics, including one or more ITAM motifs in their cytoplasmic domain, charged acidic residue in transmembrane region (critical for interaction with its partner chain) and the ability to recruit Src homology domain-2 (SH2)-containing proteins following tyrosine phosphorylation. The ITAM motif mediates signal propagation by activation of the ZAP70 or Syk tyrosine kinase. Both kinases phosphorylate several substrates, thereby facilitating the formation of a signaling complex leading to cellular activation. Interestingly, some B-cells and T-cells also express DAP12 under inflammatory conditions. In humans, subsets of CD4⁺CD28⁻ T-cells, αβTCR⁺CD4⁺ T-cells, and CD8⁺ T-cells expressing this protein have been described in patients suffering from chronic inflammatory diseases, in the context of autoimmune T cells (Schleinitz, N. et al., PLoS ONE, 4 (2009), p. e6264). In view of the significant level of DAP12 expression in mouse peritoneal macrophages, this protein is believed to be expressed in other macrophage-related cells, such as osteoclasts in the bone marrow, Kupffer cells in the liver, alveolar macrophages of the lung, Langerhans cells of skin, and microglial cells in the brain (Takaki, R et al., Immunol Rev, 2006. 214: p. 118-29).

TREM2 has been identified as expressed on the surface of human monocyte-derived dendritic cells and as an mRNA transcript in the mouse macrophage cell line RAW264 (Bouchon, A et al., J Exp Med, 2001. 194(8): p. 1111-22). Human TREM2 was the first DAP12-associated receptor described on the surface of DCs. Studies have demonstrated that TREM2 cell surface expression is reduced in DAP12-deficient bone marrow-derived dendritic cells (BMDCs) and in DAP12-deficient macrophages, as compared to wild-type cells (Ito, H and Hamerman, J A, Eur J Immunol. 42(1): p. 176-85; Hamerman, J A et al., J Immunol, 2006. 177(4): p. 2051-5; and Hamerman, J A et al., Nat Immunol, 2005. 6(6): p. 579-86). This indicates that formation of the TREM2/DAP12 complex is needed for maximal TREM2 surface expression.

Recent studies have also shown cell-surface expression of TREM2 on macrophages infiltrating tissue from the circulation, as well as on macrophages activated by IL-4 or IL-13 (Turnbull, I R et al., J Immunol, 2006. 177(6): p. 3520-4). However, TREM2 expression was not always found in other cell populations, such as tissue-resident macrophages, circulating monocytes, or the corresponding progenitor cells in the bone marrow, suggesting that TREM2 expression is not induced centrally, but locally during tissue infiltration or by cytokine-mediated activation. Moreover, it has also been observed that IFN-γ and LPS reduce TREM2 expression. Further, it has been recently reported that TREM2 is highly expressed on microglia and infiltrating macrophages in the central nervous system during experimental autoimmune encaphalomyelitis or Alzheimer's disease (Picchio, L et al., Eur J Immunol, 2007. 37(5): p. 1290-301; and Wang Y, Cell. 2015 Mar. 12; 160(6):1061-71).

It has been shown that TREM2 signals through DAP12. Downstream this leads to activation of the Syk/Zap70 tyrosine kinase family, PI3K, and other intracellular signals. On myeloid cells, TLR signals are important for activation, such as with infection response, but also play a key role in the pathological inflammatory response, such as with macrophages and dendritic cells (Hamerman, J A et al., (2006) J Immunol 177: 2051-2055; Ito, H et al., Eur J Immunol 42: 176-185; Neumann, H et al., (2007) J Neuroimmunol 184: 92-99; Takahashi, K et al., (2005) J Exp Med 201: 647-657; and Takahashi, K et al., (2007) PLoS Med 4: e124). Deficiency of either TREM2 or DAP12 is thought to lead to increased pro-inflammatory signaling. The impact of TREM2-deficiency in vitro has been shown in the context of stimulation with typical TLR ligands, such as LPS, CpG DNA, and Zymosan. TREM-2-deficient dendritic cells show increased release of IL-12p70, TNF, IL-6, and IL-10 in the presence, but not in the absence of stimulation.

Several recent studies have explored the intracellular signaling events induced by the activation of the TREM2/DAP12 pathway. For example, TREM2 is thought to activate signaling pathways involved in cell survival (e.g., protein kinase B-Akt), cell activation and differentiation (e.g., Syk, Erk1/2, PLC-γ, etc.), and in the control of the actin cytoskeleton (e.g., Syk, Vav, etc.) (Peng, Q et al., Sci Signal. 3(122): p. ra38; and Whittaker, G C et al., J Biol Chem. 285(5): p. 2976-85). After ligation of TREM2, the ITAM tyrosines in DAP12 are phosphorylated by SRC-family kinases leading to the recruitment and activation of the Syk kinase and/or ZAP70 kinase. In the mouse, Syk may be the predominant kinase involved, whereas in humans both Syk and ZAP70 appear to couple efficiently with such ITAM-containing subunits, binding them through their tandem SH2 domains.

Studies on TREM2 signaling have shown that, like TREM1, TREM2-mediated signaling through DAP12 also leads to an increase in intracellular calcium ion levels and ERK1/2 phosphorylation of ERK1/2 (Bouchon, A et al., J Exp Med, 2001. 194(8): p. 1111-22; and Sharif, 0 and Knapp, S, Immunobiology, 2008. 213(9-10): p. 701-13). Importantly, TREM2 receptor ligation may not induce the degradation of IkB-a and the subsequent nuclear translocation of NF-kB, which points to a possible difference between TREM2 and TREM1 signaling (Bouchon, A et al., J Exp Med, 2001. 194(8): p. 1111-22). Receptor cross-linking of TREM2 on immature dendritic cells triggers the up-regulation of molecules involved in T-cell co-stimulation, such as CD86, CD40, and MHC class II, as well as the up-regulation of the chemokine receptor CCR7 (Bouchon, A et al., J Exp Med, 2001. 194(8): p. 1111-22). TREM2 is also expressed on microglia, where receptor cross-linking results in an increase in ERK1/2 phosphorylation and CCR7, but not an increase in CD86 or MHC class II expression, suggesting possible cell type-specific differences in TREM2 signaling. Additionally, over expression of TREM2 signaling in microglia, myeloid Precursors, CHO or EK293 cells results in an increase in phagocytosis of apoptotic neurons, nerve and non-nerve tissue debris in the nervous system, disease causing proteins, bacteria and other foreign invaders, which is accompanied by a polarization and re-organization of F-actin in an ERK-dependent manner (Takahashi, K et al., PLoS Med, 2007. 4(4): p. e124; Neumann, H and Takahashi, K, J Neuroimmunol, 2007. 184(1-2): p. 92-9; and Kleinberg et al., Sci Transl Med. 2014 Jul. 2; 6(243):243ra86). However in some physiological contexts such as Pneumococcal Pneumonia, TREM2 appear to decrease phagocytosis. Thus TREM2 deficient alveolar macrophages display augmented bacterial clearance from the lung and enhanced phagocytosis of bacteria in vivo (Sharif et al., PLoS Pathog. 2014 Jun. 12; 10(6):e1004167).

It has also been shown that bone marrow-derived macrophages (BMDM) that have been silenced for TREM2 using shRNAi display increased secretion of TNF in response to the TLR2/6 ligand zymosan and the TLR9 ligand CpG, as compared to control BMDM cells that were treated with a non-specific shRNAi, indicating that TREM2 negatively regulates cytokine synthesis in macrophages (Ito, H and Hamerman, J A, Eur J Immunol. 42(1): p. 176-85; Hamerman, J A et al., J Immunol, 2006. 177(4): p. 2051-5; and Hamerman, J A et al., Nat Immunol, 2005. 6(6): p. 579-86). These results have been confirmed using BMDM cells from TREM2 knockout mice, and have further shown that levels of TNF and IL-6 were also higher in TREM2^{-/-} BMDM cells in response to LPS, as compared to wild-type BMDM cells (Turnbull, I R, et al., J Immunol, 2006. 177(6): p. 3520-4; and Turnbull, I R and Colonna, M, Nat Rev Immunol, 2007. 7(2): p. 155-61). Additionally, TREM2 overexpression in microglia has been demonstrated to lead to a decrease in TNF and inducible nitric oxide (iNOS) mRNA after culture of these cells with apoptotic neurons, whereas TREM2 knockdown resulted in a modest increase in TNF and iNOS mRNA levels. This indicates that, in contrast to TREM1, which is a positive regulator of cytokine synthesis, TREM2 is a negative regulator of cytokine synthesis. This effect of TREM2 on inflammation was thought to be independent of the type of macrophage as it occurs in both microglia and BMDM cells.

It has also been shown that in resident myeloid cells of the central nervous system, activation of microglia can lead to inflammation (Neumann, H et al., (2007) J Neuroimmunol 184: 92-99; Takahashi, K et al., (2005) J Exp Med 201: 647-657; Takahashi, K et al., (2007) PLoS Med 4: e124; and Hsieh, C L et al., (2009) J Neurochem 109: 1144-1156). Moreover, microglia activation has also been implicated in frontotemporal dementia (FTD), Alzheimer's disease, Parkinson's disease, stroke/ischemic brain injury, and multiple sclerosis. Whereas reduced TREM2 activation leads to increases in certain activation and inflammation markers, such as NOS2 gene transcription in myeloid cells, increased TREM2 activation leads to reduced NOS2 transcription. It is thought that dying neurons express an endogenous ligand for TREM2. HSP60 has been implicated as a ligand of TREM2 on neuroblastoma cells (Stefani, L et al., (2009) Neurochem 110: 284-294). TREM2 over-expression also leads to increased phagocytosis of dying neurons by microglia, and similarly increases phagocytosis by other myeloid lineage cells. TREM2 has also been implicated in myeloid cell migration, as TREM2 deficient myeloid cells fail to populate the brain of rodent models for Alzheimer's disease (Malm, T M et al, Neurotherapeutics. 2014 Nov. 18).

In humans, the complete absence of TREM2 has been shown to cause Nasu-Hakola disease, a rare neurodegenerative disease with late-onset dementia, demyelination, and cerebral atrophy (Paloneva, J et al., (2002) Am J Hum Genet 71: 656-662; and Paloneva, J et al., (2003) J Exp Med 198: 669-675). Nasu-Hakola disease can also be caused by DAP12-deficiency. Further, exome sequencing of individuals with frontotemporal dementia (FTD) presentation has identified homozygous mutations in TREM2 (Guerreiro, R J et al., (2013) JAMA Neurol 70: 78-84; Guerreiro, R J et al., (2012) Arch Neurol: 1-7). More recently, heterozygous mutations in TREM2 were found to increase the risk of Alzheimer's disease by up to 3 fold (Guerreiro, R et al., (2013) N Engl J Med 368: 117-127; Jonsson, T et al., (2013) N Engl J Med 368: 107-116; and Neumann, H et al., (2013) N Engl J Med 368: 182-184). Even individuals without Alzheimer's disease who carry a heterozygous TREM2 mutation show worse cognition as compared to individuals with two normal TREM2 alleles. These carriers also display doubling in the rate of brain volume shrinkage (Rajagopalan et al., (2013) N Engl J Med 369; 16). Some of these mutations lead to truncation and likely loss-of-function of TREM2. While others involve changes in amino acids including, Q33X, R47H, T66M, and S116C (Borroni B, et al. Neurobiol Aging. 2014 April; 35(4):934.e7-10). Imaging analysis in certain individuals with TREM2 homozygous mutations has also shown evidence of demyelination. Further, it has been shown that the R47H variant of TREM2 (arginine to histidine amino acid substitution at position 47 of TREM2), which is the most common TREM2 mutation, is located within the immunoglobulin domain of TREM2 and reduces ligand binding. Other TREM2 mutations were shown to reduce cell surface expression of TREM2 indicating that loss of function is the cause of increased risk for AD (Wang Y, Cell. 2015; 160(6):1061-71).

In addition an integrative network-based approach to rank-ordered organized structure of molecular networks of gene expression for relevance to late onset developing Alzheimer's disease (LOAD) identified TYROBP/DAP12 as the signaling molecule for TREM2 as a key regulator of the immune/microglia gene modules that is associated with LOAD. TYROBP was found to be the causal regulator of the highest scoring immune/microglia module as rank-ordered based on the number of other genes that TREM2 regulated and the magnitude of loss of regulation, as well as differential expression in LOAD brains. TYROBP was significantly upregulated in LOAD brains and there was a progression of TYROBP expression changes across mild cognitive impairment (MCI), which often precedes LOAD (Zhang et al., (2013) Cell 153, 707-720; and Ma et al., Mol Neurobiol. 2014 Jul. 23). Targeting such causal networks in ways that restore them to a normal state may be a way to treat disease.

TREM2 is highly expressed on microglia and infiltrating macrophages in the central nervous system during pathological conditions including Alzheimer's disease (Picchio, L et al., (2007) Eur J Immunol, 37(5): p. 1290-301; and Wang et al., (2015), Cell.; 160(6):1061-71). TREM2 gene expression has also been shown to be increased in APP23 transgenic mice, an Alzheimer's disease model in which the mice express a mutant form of the amyloid precursor protein that is associated with familial Alzheimer's disease (Melchior, B et al., ASN Neuro 2: e00037). Uptake of Amyloid 1-42 has also been shown to be increased in BV-2 microglial cell lines that overexpress TREM2.

TREM2 has further been shown to be upregulated in the EAE mouse model of multiple sclerosis (Neumann, H et al., (2007) J Neuroimmunol 184: 92-99; Takahashi, K et al., (2005) J Exp Med 201: 647-657; and Takahashi, K et al., (2007) PLoS Med 4: e124). The transduction of bone marrow-derived myeloid precursor cells (BM-DC) in vitro with TREM2 leads to increased phagocytosis of beads or of neuron fragments. In response to LPS, these cells show increased IL-10 and decreased IL-1β. Intravenous transplantation of myeloid cells overexpressing TREM2 can suppress EAE in vivo. Conversely, deficiency in TREM2 was shown to exacerbate Multiple Sclerosis in a Cuprizon model of the disease (Cantoni et al., Acta Neuropathol (2015)129(3):429-47; Luigi Poliani et al., (2015) J Clin Invest. 125(5):2161-2170). Deficiency in TREM2 was also shown to also exacerbate Alzheimer's disease in a rodent model (Wang et al., (2015), Cell.; 160(6):1061-71), although opposing data showing beneficial effect of TREM2 deficiency Alzheimer's disease in a rodent model have also been reported (Jay et al., (2015) J Exp Med 212:287-295). TREM2 was also shown to be required for survival of microglia in the brain (Otero et al., (2009) Nat Immunol.; 10:734-43). In summary, TREM2 variants were identified as genetic risk factors for frontotemporal dementia, Parkinson's disease, and amyotrophic lateral sclerosis (Borroni B, et al. Neurobiol Aging. 2014 April; 35(4):934.e7-10; Rayaprolu S, et al., Mol Neurodegener. 2013 Jun. 21; 8:19; and Cady J, et al., JAMA Neurol. 2014 April; 71(4):449-53). This common genetic linkage suggests a more general role for TREM2 in modulating neurodegenerative disease pathology.

TREM2 antibodies have been described, but the only reported effects are on cultured cells and their therapeutic utility is limited in part because they block interaction between TREM2 and its natural ligands, and act as antagonists in solution. Such antibodies in solution would mimic the disease causing loss of function phenotype of TREM2 mutations and would therefor pose a safety and efficacy risk. Another problem with existing anti-TREM2 antibodies is their requirement to be clustered by coating on a plastic plate or by secondary antibodies in order to induce agonistic activity. Accordingly, there is a need for antibodies that specifically bind TREM2 on a cell surface and that modulate (e.g., activate) one or more TREM2 activities in a safe and effective way in order to treat one or more diseases, disorders, and conditions associated with decreased TREM2 activity.

Some diseases may require TREM2 blocking antibodies that do not activate TREM2 under any circumstances. For example, the tumor microenvironment is composed of a heterogeneous immune infiltrate, which include T lymphocytes, macrophages and cells of myeloid/granulocytic lineage. Therapeutic approaches that modulate specific subsets of immune cells are changing the standard of care. “Checkpoint blocking” antibodies targeting immune-modulatory molecules expressed on T cells (such as CTLA-4 and PD-1) have demonstrated clinical activity across a variety of tumor types (Naidoo—et al., (2014) British Journal of Cancer 111, 2214-2219).

Cancer immune-therapy targeting tumor-associated macrophages (e.g., M2-type macrophages) is an intense area of research. The presence of M2-macrophages in tumors is associated with poor prognosis.

Accordingly, there is also a need for antibodies that specifically bind TREM2 on a cell surface and modulate (e.g., inhibit and/or otherwise reduce) ligand binding and/or one or more TREM2 activities in order to prevent, reduce the risk of, or treat cancer.

All references cited herein, including patent applications and publications, are hereby incorporated by reference in their entirety.

### SUMMARY OF THE PRESENT DISCLOSURE

The present disclosure is generally directed to compositions that include antibodies, e.g., monoclonal, chimeric, humanized antibodies, antibody fragments, etc., that specifically bind a TREM2 protein, e.g., a mammalian TREM2 (e.g., any non-human mammal) or human TREM2, and to methods of using such compositions. The antibodies of the present disclosure may include agonist, antagonist, or inert antibodies. The methods provided herein find use in preventing, reducing risk, or treating an individual having dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, solid and blood cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2 and/or TREM2 ligands, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza. The methods provided herein also find use in inducing or promoting the survival, maturation, functionality, migration, or proliferation of one or more immune cells in an individual in need thereof. The methods provided herein find further use in decreasing the activity, functionality, or survival of regulatory T cells, tumor-imbedded immunosuppressor dendritic cells, tumor-imbedded immunosuppressor macrophages, neutrophils, natural killer (NK) cells, myeloid-derived suppressor cells, tumor-associated macrophages, neutrophils, NK cells, acute myeloid leukemia (AML) cells, chronic lymphocytic leukemia (CLL) cell, or chronic myeloid leukemia (CML) cell in an individual in need thereof

In some embodiments, tumor cells, such as acute myeloblastic leukemia (AML) cell, express TREM2. Accordingly, anti-TREM2 antibodies of the present disclosure also find use in treating cancers. In some embodiments, anti-TREM2 antibodies, including antibodies that display antibody-dependent cell-mediated cytotoxicity (ADCC) and/or TREM2 antibody drug conjugates, can be used to target and inhibit cancer, such as AML.

Certain aspects of the present disclosure are based, at least in part, on the identification of two distinct classes of isolated antibodies that specifically bind to and modulate TREM2 proteins.

One class of antibodies relates to agonist antibodies that induce one or more TREM2 activities on, for example, human primary immune cells and TREM2-expressing cell lines, and when combined with one or more TREM2 ligands enhance one or more TREM2 activities induced by binding of the one or more TREM2 ligands to the TREM2 protein. Advantageously, such agonist anti-TREM2 antibodies can enhance ligand-induced TREM2 activity without competing with our otherwise blocking binding of the one or more TREM2 ligands to the TREM2 protein. In some embodiments, the agonist antibodies can activate and/or enhance the one or more TREM2 activities regardless of whether the antibodies are clustered or in solution. In some embodiments, the agonist antibodies can activate TREM2 in solution without the need to be clustered by secondary antibodies, by Fc receptors, or by binding to plates. In some embodiments, the agonist antibodies may activate TREM2 regardless of whether the mechanism for antibody clustering are present at the therapeutic site of action in vivo. In some embodiments, the agonist antibodies may have increased safety and efficacy. In some embodiments, the agonist antibodies can ensure that immune cells that express TREM2 will act primarily in the location where they are required for therapeutic efficacy and will be able to interact with their physiological targets. In some embodiments, the agonist antibodies do not block TREM2 activity that leads to increased disease risks similar to those observed with genetic mutations that reduce TREM2 activity.

The second class of antibodies relates to antagonist antibodies that specifically bind to and inhibit TREM2, and are incapable of activating TREM2 regardless of whether the antibodies are clustered or in solution. In some embodiments, the antagonist antibodies have increases safety and efficacy. In some embodiments, the antagonist antibodies are incapable of activating TREM2 regardless of their configuration or their ability to cluster.

Accordingly, certain aspects of the present disclosure relate to an isolated (e.g., monoclonal) antibody that binds to a TREM2 protein, wherein the antibody induces one or more TREM2 activities and enhances one or more TREM2 activities induced by binding of one or more TREM2 ligands to the TREM2 protein. In some embodiments, the antibody enhances one or more TREM2 activities induced by binding of one or more TREM2 ligands to the TREM2 protein, as compared to the one or more TREM2 activities induced by binding of the one or more TREM2 ligands to the TREM2 protein in the absence of the isolated antibody. In some embodiments, the antibody enhances the one or more TREM2 activities without blocking binding of the one or more TREM2 ligands to the TREM2 protein. In some embodiments, the antibody does not compete with the one or more TREM2 ligands for binding to the TREM2 protein. In some embodiments, the antibody enhances binding of the one or more TREM2 ligands to the TREM2 protein.

Other aspects of the present disclosure relate to an isolated (e.g., monoclonal) antibody that binds to a TREM2 protein, wherein the antibody induces one or more TREM2 activities without blocking binding of one or more TREM2 ligands to the TREM2 protein. In some embodiments, the antibody does not compete with the one or more TREM2 ligands for binding to the TREM2 protein. In some embodiments, the antibody enhances binding of the one or more TREM2 ligands to the TREM2 protein. In some embodiments, the antibody enhances one or more TREM2 activities induced by binding of the one or more TREM2 ligands to the TREM2 protein. In some embodiments, the antibody enhances one or more TREM2 activities induced by binding of the one or more TREM2 ligands to the TREM2 protein, as compared to the one or more TREM2 activities induced by binding of the one or more TREM2 ligands to the TREM2 protein in the absence of the isolated antibody.

In some embodiments that may be combined with any of the preceding embodiments, the antibody synergizes with the one or more TREM2 ligands to enhance the one or more TREM2 activities. In some embodiments that may be combined with any of the preceding embodiments, the antibody synergizes with the one or more TREM2 ligands to enhance the one or more TREM2 activities. In some embodiments that may be combined with any of the preceding embodiments, the antibody enhances the one or more TREM2 activities in the absence of cell surface clustering of TREM2. In some embodiments that may be combined with any of the preceding embodiments, the antibody enhances the one or more TREM2 activities by inducing or retaining cell surface clustering of TREM2. In some embodiments that may be combined with any of the preceding embodiments, the antibody is clustered by an Fc-gamma receptor expressed on one or more immune cells. In some embodiments that may be combined with any of the preceding embodiments, the one or more immune cells are B cells or microglial cells. In some embodiments that may be combined with any of the preceding embodiments, the enhancement of the one or more TREM2 activities induced by binding of one or more TREM2 ligands to the TREM2 protein is measured on primary cells selected from the group consisting of dendritic cells, bone marrow-derived dendritic cells, monocytes, microglia, macrophages, neutrophils, NK cells, osteoclasts, Langerhans cells of skin, and Kupffer cells, or on cell lines, and wherein the enhancement of the one or more TREM2 activities induced by binding of one or more TREM2 ligands to the TREM2 protein is measured utilizing an in vitro cell assay. In some embodiments that may be combined with any of the preceding embodiments, the antibody increases levels of soluble TREM2, increases half-life of soluble TREM2, or both. In some embodiments that may be combined with any of the preceding embodiments, the levels of soluble TREM2 are selected from the group consisting of serum levels of TREM2, cerebral spinal fluid (CSF) levels of TREM2, tissue levels of TREM2, and any combination thereof. In some embodiments that may be combined with any of the preceding embodiments, the antibody does not bind to soluble TREM2. In some embodiments that may be combined with any of the preceding embodiments, the antibody does not bind to soluble TREM2 in vivo. In some embodiments that may be combined with any of the preceding embodiments, the soluble TREM2 corresponds to amino acid residues selected from the group consisting of amino acid residues 19-160 of SEQ ID NO: 1, amino acid residues 19-159 of SEQ ID NO: 1, amino acid residues 19-158 of SEQ ID NO: 1, amino acid residues 19-157 of SEQ ID NO: 1, amino acid residues 19-156 of SEQ ID NO: 1, amino acid residues 19-155 of SEQ ID NO: 1, and amino acid residues 19-154 of SEQ ID NO: 1. In some embodiments that may be combined with any of the preceding embodiments, the antibody decreases levels of TREM2 in one or more cells. In some embodiments that may be combined with any of the preceding embodiments, the antibody decreases cell surface levels of TREM2, decreases intracellular levels of TREM2, decreases total levels of TREM2, or any combination thereof. In some embodiments that may be combined with any of the preceding embodiments, the antibody induces TREM2 degradation, TREM2 cleavage, TREM2 internalization, TREM2 shedding, downregulation of TREM2 expression, or any combination thereof. In some embodiments that may be combined with any of the preceding embodiments, the levels of TREM2 in one or more cells are measured in primary cells selected from the group consisting of dendritic cells, bone marrow-derived dendritic cells, monocytes, microglia, macrophages, neutrophils, NK cells, osteoclasts, Langerhans cells of skin, and Kupffer cells, or on cell lines, and wherein the cellular levels of TREM2 are measured utilizing an in vitro cell assay. In some embodiments that may be combined with any of the preceding embodiments, the TREM2 protein is a mammalian, such as a non-human mammal, protein or a human protein. In some embodiments that may be combined with any of the preceding embodiments, the TREM2 protein is a wild-type protein. In some embodiments that may be combined with any of the preceding embodiments, the TREM2 protein is a naturally occurring variant. In some embodiments that may be combined with any of the preceding embodiments, the TREM2 protein is expressed on human dendritic cells, human macrophages, human monocytes, human osteoclasts, human Langerhans cells of skin, human Kupffer cells, human microglia, or any combination thereof. In some embodiments that may be combined with any of the preceding embodiments, the one or more TREM2 activities are selected from the group consisting of: (a) TREM2 binding to DAP12; (b) TREM2 phosphorylation; (c) DAP12 phosphorylation; (d) activation of one or more tyrosine kinases, optionally wherein the one or more tyrosine kinases comprise a Syk kinase, ZAP70 kinase, or both; (e) activation of phosphatidylinositol 3-kinase (PI3K); (0 activation of protein kinase B (Akt); (g) recruitment of phospholipase C-gamma (PLC-gamma) to a cellular plasma membrane, activation of PLC-gamma, or both; (h) recruitment of TEC-family kinase dVav to a cellular plasma membrane; (i) activation of nuclear factor-rB (NF-rB); (j) inhibition of MAPK signaling; (k) phosphorylation of linker for activation of T cells (LAT), linker for activation of B cells (LAB), or both; (1) activation of IL-2-induced tyrosine kinase (Itk); (m) modulation of one or more pro-inflammatory mediators selected from the group consisting of IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (n) modulation of one or more anti-inflammatory mediators selected from the group consisting of IL-4, IL-10 TGF-β, IL-13, IL-35 IL-16, IFN-alpha, IL-1Ra, VEGF, G-CSF, YM, AXL, FLT1, and soluble receptors for TNF or IL-6, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (o) modulation of one or more genes whose expression is increased upon induction of inflammation, optionally wherein the one or more genes are selected from the group consisting of Fabp3, Fabp5, and LDR; (p) phosphorylation of extracellular signal-regulated kinase (ERK); (q) modulated expression of C—C chemokine receptor 7 (CCR7) in one or more cells selected from the group consisting of macrophages, M1 macrophages, activated M1 macrophages, M2 macrophages, dendritic cells, monocytes, osteoclasts, Langerhans cells of skin, Kupffer cells, microglia, M1 microglia, activated M1 microglia, and M2 microglia, and any combination thereof; (r) induction of microglial cell chemotaxis toward CCL19 and CCL21 expressing cells; (s) normalization of disrupted TREM2/DAP12-dependent gene expression; (t) recruitment of Syk, ZAP70, or both to a DAP12/TREM2 complex; (u) increasing activity of one or more TREM2-dependent genes, optionally wherein the one or more TREM2-dependent genes comprise nuclear factor of activated T-cells (NFAT) transcription factors; (v) increased maturation of dendritic cells, monocytes, microglia, M1 microglia, activated M1 microglia, and M2 microglia, macrophages, M1 macrophages, activated M1 macrophages, M2 macrophages, or any combination thereof; (w) increased ability of dendritic cells, monocytes, microglia, M1 microglia, activated M1 microglia, and M2 microglia, macrophages, M1 macrophages, activated M1 macrophages, M2 macrophages, or any combination thereof to prime or modulate the function of T cells, optionally wherein the T cells are one or more cells selected from the group consisting of CD8+ T cells, CD4+ T cells regulatory T cells, and any combination thereof; (x) enhanced ability, normalized ability, or both of bone marrow-derived dendritic cells to prime or modulate function of antigen-specific T cells, optionally wherein the antigen-specific T cells are one or more cells selected from the group consisting of CD8+ T cells, CD4+ T cells regulatory T cells, and any combination thereof; (y) enhanced ability, normalized ability, or both of bone marrow-derived dendritic cells to induce antigen-specific T-cell proliferation; (z) induction of osteoclast production, increased rate of osteoclastogenesis, or both; (aa) increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; (bb) increasing the function of dendritic cells, macrophages, M1 macrophages, activated M1 macrophages, M2 macrophages, microglia, M1 microglia, activated M1 microglia, and M2 microglia, or any combination thereof; (cc) increasing phagocytosis by dendritic cells, macrophages, M1 macrophages, activated M1 macrophages, M2 macrophages, monocytes, microglia, M1 microglia, activated M1 microglia, and M2 microglia, or any combination thereof; (dd) induction of one or more types of clearance selected from the group consisting of apoptotic neuron clearance, nerve tissue debris clearance, non-nerve tissue debris clearance, bacteria or other foreign body clearance, disease-causing agent clearance, tumor cell clearance, or any combination thereof, optionally wherein the disease-causing agent is selected from the group consisting of amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products including DiPeptide Repeats, (DPRs peptides) composed of glycine-alanine (GA), glycine-proline (GP), glycine-arginine (GR), proline-alanine (PA), or proline-arginine (PR), antisense GGCCCC (G2C4) repeat-expansion RNA; (ee) induction of phagocytosis of one or more of apoptotic neurons, nerve tissue debris, non-nerve tissue debris, bacteria, other foreign bodies, disease-causing agents, tumor cells, or any combination thereof, optionally wherein the disease-causing agent is selected from the group consisting of amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products including DiPeptide Repeats, (DPRs peptides) composed of glycine-alanine (GA), glycine-proline (GP), glycine-arginine (GR), proline-alanine (PA), or proline-arginine (PR), antisense GGCCCC (G2C4) repeat-expansion RNA; (ff) modulated expression of one or more stimulatory molecules selected from the group consisting of CD83, CD86 MHC class II. CD40, and any combination thereof, optionally wherein the CD40 is expressed on dendritic cells, monocytes, macrophages, or any combination thereof, and optionally wherein the dendritic cells comprise bone marrow-derived dendritic cells; (gg) modulating secretion of one or more pro-inflammatory mediators selected from the group consisting of IFN-β, IL-1α, IL-1β, CD86, TNF-α, IL-6, IL-8, CRP, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, and optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (hh) modulating secretion of one or more anti-inflammatory mediators selected from the group consisting of IL-4, IL-10 TGF-β, IL-13, IL-35 IL-16, IFN-alpha, IL-1Ra, VEGF, G-CSF, YM, AXL, FLT1, and soluble receptors for TNF or IL-6, and optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (ii) modulating expression of one or more proteins selected from the group consisting of C1qa, C1qB, C1qC, C1s, C1R, C4, C2, C3, ITGB2, HMOX1, LAT2. CASP1, CSTA, VSIG4, MS4A4A, C3AR1, GPX1, TyroBP, ALOXSAP, ITGAM, SLC7A7, CD4, ITGAX, PYCARD, and VEGF; (jj) increasing memory; and (kk) reducing cognitive deficit. In some embodiments that may be combined with any of the preceding embodiments, the one or more TREM2 activities are selected from the group consisting of: (a) TREM2 binding to DAP12; (b) DAP12 phosphorylation; (c) activation of Syk kinase; (d) modulation of one or more pro-inflammatory mediators selected from the group consisting of IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (e) recruitment of Syk to a DAP12/TREM2 complex; (f) increasing activity of one or more TREM2-dependent genes, optionally wherein the one or more TREM2-dependent genes comprise nuclear factor of activated T-cells (NFAT) transcription factors; (g) increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; (h) modulated expression of one or more stimulatory molecules selected from the group consisting of CD83, CD86 MHC class II, CD40, and any combination thereof, optionally wherein the CD40 is expressed on dendritic cells, monocytes, macrophages, or any combination thereof, and optionally wherein the dendritic cells comprise bone marrow-derived dendritic cells; (i) increasing memory; and (j) reducing cognitive deficit. In some embodiments that may be combined with any of the preceding embodiments, the antibody is of the IgG class the IgM class, or the IgA class. In some embodiments that may be combined with any of the preceding embodiments, the antibody is of the IgG class and has an IgG1, IgG2, IgG3, or IgG4 isotype. In some embodiments that may be combined with any of the preceding embodiments, the antibody has an IgG2 isotype. In some embodiments that may be combined with any of the preceding embodiments, the antibody comprises a human IgG2 constant region. In some embodiments that may be combined with any of the preceding embodiments, the human IgG2 constant region comprises an Fc region. In some embodiments that may be combined with any of the preceding embodiments, the antibody enhances the one or more TREM2 activities independently of binding to an Fc receptor. In some embodiments that may be combined with any of the preceding embodiments, the antibody binds an inhibitory Fc receptor. In some embodiments that may be combined with any of the preceding embodiments, the inhibitory Fc receptor is inhibitory Fc-gamma receptor IIB (FcγIIB). In some embodiments that may be combined with any of the preceding embodiments: (a) the isolated antibody has a human or mouse IgG1 isotype and comprises one or more amino acid substitutions in the Fc region at a residue position selected from the group consisting of: N297A, D265A, D270A, L234A, L235A, G237A, C226S, C229S, E233P, L234V, L234F, L235E, P331S, S267E, L328F, A330L, M252Y, S254T, T256E, L328E, P238D, S267E, L328F, E233D, G237D, H268D, P271G, A330R, and any combination thereof, wherein the numbering of the residues is according to EU numbering, or comprises an amino acid deletion in the Fc region at a position corresponding to glycine 236; (b) the isolated antibody has an IgG1 isotype and comprises an IgG2 isotype heavy chain constant domain 1(CH1) and hinge region, optionally wherein the IgG2 isotype CH1 and hinge region comprises the amino acid sequence of ASTKGPSVFP LAPCSRSTSE STAALGCLVK DYFPEPVTVS WNSGALTSGVHTFPAVLQSS GLYSLSSVVT VPSSNFGTQT YTCNVDHKPS NTKVDKTVERKCCVECPPCP (SEQ ID NO: 886), and optionally wherein the antibody Fc region comprises a S267E amino acid substitution, a L328F amino acid substitution, or both, and/or a N297A or N297Q amino acid substitution, wherein the numbering of the residues is according to EU numbering; (c) the isolated antibody has an IgG2 isotype and comprises one or more amino acid substitutions in the Fc region at a residue position selected from the group consisting of: P238S, V234A, G237A, H268A, H268Q, V309L, A330S, P331S, C214S, C232S, C233S, S267E, L328F, M252Y, S254T, T256E, H268E, N297A, N297Q, A330L, and any combination thereof, wherein the numbering of the residues is according to EU numbering; (d) the a isolated antibody has a human or mouse IgG4 isotype and comprises one or more amino acid substitutions in the Fc region at a residue position selected from the group consisting of: L235A, G237A, S228P, L236E, S267E, E318A, L328F, M252Y, S254T, T256E, E233P, F234V, L234A/F234A, S228P, S241P, L248E, T394D, N297A, N297Q, L235E, and any combination thereof, wherein the numbering of the residues is according to EU numbering; or (e) the isolated antibody has a hybrid IgG2/4 isotype, and optionally wherein the antibody comprises an amino acid sequence comprising amino acids 118 to 260 of human IgG2 and amino acids 261 to 447 of human IgG4, wherein the numbering of the residues is according to EU or numbering. In some embodiments that may be combined with any of the preceding embodiments, the antibody has an IgG4 isotype. In some embodiments that may be combined with any of the preceding embodiments, the antibody comprises an S228P amino acid substitution at residue position 228, an F234A amino acid substitution at residue position 234, and an L235A amino acid substitution at residue position 235, wherein the numbering of the residue position is according to EU numbering.

In some embodiments that may be combined with any of the preceding embodiments, the antibody binds to one or more amino acids within amino acid residues selected from the group consisting of: (i) amino acid residues 19-174 of SEQ ID NO: 1, or amino acid residues on a TREM2 protein corresponding to amino acid residues 19-174 of SEQ ID NO: 1; (ii) amino acid residues 29-112 of SEQ ID NO: 1, or amino acid residues on a TREM2 protein corresponding to amino acid residues 29-112 of SEQ ID NO: 1; (iii) amino acid residues 113-174 of SEQ ID NO: 1, or amino acid residues on a TREM2 protein corresponding to amino acid residues 113-174 of SEQ ID NO: 1; (iv) amino acid residues 35-49 of SEQ ID NO: 1, or amino acid residues on a TREM2 protein corresponding to amino acid residues 35-49 of SEQ ID NO: 1; (v) amino acid residues 35-49 and 140-150 of SEQ ID NO: 1, or amino acid residues on a TREM2 protein corresponding to amino acid residues 35-49 and 140-150 of SEQ ID NO: 1; (vi) amino acid residues 39-49 of SEQ ID NO: 1, or amino acid residues on a TREM2 protein corresponding to amino acid residues 39-49 of SEQ ID NO: 1; (vii) amino acid residues 39-49 and 63-77 of SEQ ID NO: 1, or amino acid residues on a TREM2 protein corresponding to amino acid residues 39-49 and 63-77 of SEQ ID NO: 1; (viii) amino acid residues 51-61 of SEQ ID NO: 1, or amino acid residues on a TREM2 protein corresponding to amino acid residues 51-61 of SEQ ID NO: 1; (ix) amino acid residues 55-62 of SEQ ID NO: 1, or amino acid residues on a TREM2 protein corresponding to amino acid residues 55-62 of SEQ ID NO: 1; (x) amino acid residues 55-62, 104-109, and 148-158 of SEQ ID NO: 1, or amino acid residues on a TREM2 protein corresponding to amino acid residues 55-62, 104-109, and 148-158 of SEQ ID NO: 1; (xi) amino acid residues 55-62, 104-109, and 160-166 of SEQ ID NO: 1, or amino acid residues on a TREM2 protein corresponding to amino acid residues 55-62, 104-109, and 160-166 of SEQ ID NO: 1; (xii) amino acid residues 55-65 of SEQ ID NO: 1, or amino acid residues on a TREM2 protein corresponding to amino acid residues 55-65 of SEQ ID NO: 1; (xiii) amino acid residues 55-65 and 124-134 of SEQ ID NO: 1, or amino acid residues on a TREM2 protein corresponding to amino acid residues 55-65 and 124-134 of SEQ ID NO: 1; (xiv) amino acid residues 63-73 of SEQ ID NO: 1, or amino acid residues on a TREM2 protein corresponding to amino acid residues 63-73 of SEQ ID NO: 1; (xv) amino acid residues 63-77 of SEQ ID NO: 1, or amino acid residues on a TREM2 protein corresponding to amino acid residues 63-77 of SEQ ID NO: 1; (xvi) amino acid residues 104-109 of SEQ ID NO: 1, or amino acid residues on a TREM2 protein corresponding to amino acid residues 104-109 of SEQ ID NO: 1; (xvii) amino acid residues 117-133 of SEQ ID NO: 1, or amino acid residues on a TREM2 protein corresponding to amino acid residues 117-133 of SEQ ID NO: 1; (xviii) amino acid residues 124-134 of SEQ ID NO: 1, or amino acid residues on a TREM2 protein corresponding to amino acid residues 124-134 of SEQ ID NO: 1; (xix) amino acid residues 137-146 of SEQ ID NO: 1, or amino acid residues on a TREM2 protein corresponding to amino acid residues 137-146 of SEQ ID NO: 1; (xx) amino acid residues 139-147 of SEQ ID NO: 1, or amino acid residues on a TREM2 protein corresponding to amino acid residues 139-147 of SEQ ID NO: 1; (xxi) amino acid residues 139-149 of SEQ ID NO: 1, or amino acid residues on a TREM2 protein corresponding to amino acid residues 139-149 of SEQ ID NO: 1; (xxii) amino acid residues 140-150 of SEQ ID NO: 1, or amino acid residues on a TREM2 protein corresponding to amino acid residues 140-150 of SEQ ID NO: 1; (xxiii) amino acid residues 140-146 of SEQ ID NO: 1, or amino acid residues on a TREM2 protein corresponding to amino acid residues 140-146 of SEQ ID NO: 1; (xxiv) amino acid residues 140-143 of SEQ ID NO: 1, or amino acid residues on a TREM2 protein corresponding to amino acid residues 140-143 of SEQ ID NO: 1; (xxv) amino acid residues 142-152 of SEQ ID NO: 1, or amino acid residues on a TREM2 protein corresponding to amino acid residues 142-152 of SEQ ID NO: 1; (xxvi) amino acid residues 146-154 of SEQ ID NO: 1, or amino acid residues on a TREM2 protein corresponding to amino acid residues 146-154 of SEQ ID NO: 1; (xxvii) amino acid residues 148-158 of SEQ ID NO: 1, or amino acid residues on a TREM2 protein corresponding to amino acid residues 148-158 of SEQ ID NO: 1; (xxviii) amino acid residues 149-157 of SEQ ID NO: 1, or amino acid residues on a TREM2 protein corresponding to amino acid residues 149-157 of SEQ ID NO: 1; (xxix) amino acid residues 149 and 150 of SEQ ID NO: 1, or amino acid residues on a TREM2 protein corresponding to amino acid residues 149 and 150 of SEQ ID NO: 1; (xxx) amino acid residues 151-155 of SEQ ID NO: 1, or amino acid residues on a TREM2 protein corresponding to amino acid residues 151-155 of SEQ ID NO: 1; (xxxi) amino acid residues 154-161 of SEQ ID NO: 1, or amino acid residues on a TREM2 protein corresponding to amino acid residues 154-161 of SEQ ID NO: 1; (xxxii) amino acid residues 156-170 of SEQ ID NO: 1, or amino acid residues on a TREM2 protein corresponding to amino acid residues 156-170 of SEQ ID NO: 1; (xxxiii) amino acid residues 160-166 of SEQ ID NO: 1, or amino acid residues on a TREM2 protein corresponding to amino acid residues 160-166 of SEQ ID NO: 1; and (xxxiv) amino acid residues 162-165 of SEQ ID NO: 1, or amino acid residues on a TREM2 protein corresponding to amino acid residues 162-165 of SEQ ID NO: 1. In some embodiments that may be combined with any of the preceding embodiments, the antibody binds to one or more amino acid residues selected from the group consisting of K42, H43, W44, G45, H67, R77, T88, H114, E117, E151, D152, H154, and E156 of SEQ ID NO: 1, or one or more amino acid residues on a mammalian TREM2 protein corresponding to an amino acid residue selected from the group consisting of K42, H43, W44, G45, H67, R77, T88, H114, E117, E151, D152, H154, and E156 of SEQ ID NO: 1. In some embodiments that may be combined with any of the preceding embodiments, the antibody binds to one or more amino acid residues selected from the group consisting of E151, D152, H154, and E156 of SEQ ID NO: 1, or one or more amino acid residues on a mammalian TREM2 protein corresponding to an amino acid residue selected from the group consisting of E151, D152, H154, and E156 of SEQ ID NO: 1. In some embodiments that may be combined with any of the preceding embodiments, the antibody competes with one or more antibodies selected from the group consisting of 3B10, 7B3, 8F8, 9F5, 9G1, 9G3, 11A8, 12F9, 7E9, 7F6, 8C3, 2C5, 3C5, 4C12, 7D9, 2F6, 3A7, 7E5, 11H5, 1B4, 6H2, 7B11, 18D8, 18E4, 29F6, 40D5, 43B9, 44A8, 44B4, and any combination thereof for binding to TREM2.

In some embodiments that may be combined with any of the preceding embodiments, the antibody comprises a light chain variable domain and a heavy chain variable domain, wherein the light chain variable domain, or the heavy chain variable domain, or both comprise at least one, two, three, four, five, or six HVRs selected from HVR-L1, HVR-L2, HVR-L3, HVR-H1, HVR-H2, and HVR-H3 of an antibody selected from the group consisting of: 4D11, 7C5, 6G12, 8F11, 8E10, 7E5, 7F8, 8F8, 1H7, 2H8, 3A2, 3A7, 3B10, 4F11, 6H6, 7A9, 7B3, 8A1, 9F5, 9G1, 9G3, 10A9, 11A8, 12D9, 12F9, 10C1, 7E9, 7F6, 8C3, 2C5, 3C5, 4C12, 7D9, 2F6, 11H5, B4, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2. In some embodiments that may be combined with any of the preceding embodiments: (a) the HVR-L1 comprises an amino acid sequence selected from the group consisting of SEQ ID NOs: 9-23, 581, 690-694, 734-738, and 826-828; (b) the HVR-L2 comprises an amino acid sequence selected from the group consisting of SEQ ID NOs: 24-33, 695-697, and 739-743; (c) the HVR-L3 comprises an amino acid sequence selected from the group consisting of SEQ ID NOs: 34-47, 582, 583, 698-702, and 744-746; (d) the HVR-H1 comprises an amino acid sequence selected from the group consisting of SEQ ID NOs: 48-65, 584, 703-705, 747-754, and 829-835; (e) the HVR-H2 comprises an amino acid sequence selected from the group consisting of SEQ ID NOs: 66-84, 585-587, 706-708, 755-762, 836-842, and 888; or (f) the HVR-H3 comprises an amino acid sequence selected from the group consisting of SEQ ID NOs: 85-102, 588, 589, 709, 710, and 763-770. In some embodiments that may be combined with any of the preceding embodiments: (a) the HVR-L1 comprises the amino acid sequence of SEQ ID NO: 11, the HVR-L2 comprises the amino acid sequence of SEQ ID NO: 26, the HVR-L3 comprises the amino acid sequence of SEQ ID NO: 36, the HVR-H1 comprises the amino acid sequence of SEQ ID NO: 51, the HVR-H2 comprises the amino acid sequence of SEQ ID NO: 69, and the HVR-H3 comprises the amino acid sequence of SEQ ID NO: 88; (b) the HVR-L1 comprises the amino acid sequence of SEQ ID NO: 14, the HVR-L2 comprises the amino acid sequence of SEQ ID NO: 28, the HVR-L3 comprises the amino acid sequence of SEQ ID NO: 39, the HVR-H1 comprises the amino acid sequence of SEQ ID NO: 53, the HVR-H2 comprises the amino acid sequence of SEQ ID NO: 71, and the HVR-H3 comprises the amino acid sequence of SEQ ID NO: 90; (c) the HVR-L1 comprises the amino acid sequence of SEQ ID NO: 11, the HVR-L2 comprises the amino acid sequence of SEQ ID NO: 26, the HVR-L3 comprises the amino acid sequence of SEQ ID NO: 36, the HVR-H1 comprises the amino acid sequence of SEQ ID NO: 51, the HVR-H2 comprises the amino acid sequence of SEQ ID NO: 69, and the HVR-H3 comprises the amino acid sequence of SEQ ID NO: 88; (d) the HVR-L1 comprises the amino acid sequence of SEQ ID NO: 16, the HVR-L2 comprises the amino acid sequence of SEQ ID NO: 29, the HVR-L3 comprises the amino acid sequence of SEQ ID NO: 35, the HVR-H1 comprises the amino acid sequence of SEQ ID NO: 55, the HVR-H2 comprises the amino acid sequence of SEQ ID NO: 73, and the HVR-H3 comprises the amino acid sequence of SEQ ID NO: 92; (e) the HVR-H1 comprises the amino acid sequence of SEQ ID NO: 58, the HVR-H2 comprises the amino acid sequence of SEQ ID NO: 76, and the HVR-H3 comprises the amino acid sequence of SEQ ID NO: 95; (f) the HVR-L1 comprises the amino acid sequence of SEQ ID NO: 19, the HVR-L2 comprises the amino acid sequence of SEQ ID NO: 28, the HVR-L3 comprises the amino acid sequence of SEQ ID NO: 43, the HVR-H1 comprises the amino acid sequence of SEQ ID NO: 60, the HVR-H2 comprises the amino acid sequence of SEQ ID NO: 78, and the HVR-H3 comprises the amino acid sequence of SEQ ID NO: 97; (g) the HVR-L1 comprises the amino acid sequence of SEQ ID NO: 20, the HVR-L2 comprises the amino acid sequence of SEQ ID NO: 28, the HVR-L3 comprises the amino acid sequence of SEQ ID NO: 44, the HVR-H1 comprises the amino acid sequence of SEQ ID NO: 61, the HVR-H2 comprises the amino acid sequence of SEQ ID NO: 79, and the HVR-H3 comprises the amino acid sequence of SEQ ID NO: 98; (h) the HVR-L1 comprises the amino acid sequence of SEQ ID NO: 21, the HVR-L2 comprises the amino acid sequence of SEQ ID NO: 32, the HVR-L3 comprises the amino acid sequence of SEQ ID NO: 45, the HVR-H1 comprises the amino acid sequence of SEQ ID NO: 62, the HVR-H2 comprises the amino acid sequence of SEQ ID NO: 80, and the HVR-H3 comprises the amino acid sequence of SEQ ID NO: 99; (i) the HVR-L1 comprises the amino acid sequence of SEQ ID NO: 22, the HVR-L2 comprises the amino acid sequence of SEQ ID NO: 29, the HVR-L3 comprises the amino acid sequence of SEQ ID NO: 46, the HVR-H1 comprises the amino acid sequence of SEQ ID NO: 63, the HVR-H2 comprises the amino acid sequence of SEQ ID NO: 82, and the HVR-H3 comprises the amino acid sequence of SEQ ID NO: 100; or (j) the HVR-L1 comprises the amino acid sequence of SEQ ID NO: 16, the HVR-L2 comprises the amino acid sequence of SEQ ID NO: 29, the HVR-L3 comprises the amino acid sequence of SEQ ID NO: 35, the HVR-H1 comprises the amino acid sequence of SEQ ID NO: 65, the HVR-H2 comprises the amino acid sequence of SEQ ID NO: 84, and the HVR-H3 comprises the amino acid sequence of SEQ ID NO: 102. In some embodiments that may be combined with any of the preceding embodiments, the antibody comprises a light chain variable domain and a heavy chain variable domain, wherein the light chain variable domain comprises: (a) an HVR-L1 comprising an amino acid sequence selected from the group consisting of SEQ ID NOs: 9-23, 581, 690-694, 734-738, and 826-828, or an amino acid sequence with at least about 90% homology to an amino acid sequence selected from the group consisting of SEQ ID NOs: 9-23, 581, 690-694, 734-738, and 826-828; (b) an HVR-L2 comprising an amino acid sequence selected from the group consisting of SEQ ID NOs: 24-33, 695-697, and 739-743, or an amino acid sequence with at least about 90% homology to an amino acid sequence selected from the group consisting of SEQ ID NOs: 24-33, 695-697, and 739-743; and (c) an HVR-L3 comprising an amino acid sequence selected from the group consisting of SEQ ID NOs: 34-47, 582, 583, 698-702, and 744-746, or an amino acid sequence with at least about 90% homology to an amino acid sequence selected from the group consisting of SEQ ID NOs: 34-47, 582, 583, 698-702, and 744-746; and wherein the heavy chain variable domain comprises: (a) an HVR-H1 comprising an amino acid sequence selected from the group consisting of SEQ ID NOs: 48-65, 584, 703-705, 747-754, and 829-835, or an amino acid sequence with at least about 90% homology to an amino acid sequence selected from the group consisting of SEQ ID NOs: 48-65, 584, 703-705, 747-754, and 829-835; (b) an HVR-H2 comprising an amino acid sequence selected from the group consisting of SEQ ID NOs: 66-84, 585-587, 706-708, 755-762, 836-842, and 888, or an amino acid sequence with at least about 90% homology to an amino acid sequence selected from the group consisting of SEQ ID NOs: 66-84, 585-587, 706-708, 755-762, 836-842, and 888; and (c) an HVR-H3 comprising an amino acid sequence selected from the group consisting of SEQ ID NOs: 85-102, 588, 589, 709, 710, and 763-770, or an amino acid sequence with at least about 90% homology to an amino acid sequence selected from the group consisting of SEQ ID NOs: 85-102, 588, 589, 709, 710, and 763-770. In some embodiments that may be combined with any of the preceding embodiments, the antibody comprises a light chain variable domain comprising an amino acid sequence selected from the group consisting of SEQ ID NOs: 219-398, 602-634, 679-689, 724-730, 809-816, 821, 843, 844, 849, and 850; and/or a heavy chain variable domain comprising an amino acid sequence selected from the group consisting of SEQ ID NOs: 399-580, 635-678, 731-733, and 817-820, 822-825, and 845-847. In some embodiments that may be combined with any of the preceding embodiments, the antibody comprises a light chain variable domain and a heavy chain variable domain, wherein: (a) the light chain variable domain comprises the amino acid sequence of SEQ ID NO: 333 and the heavy chain variable domain comprises the amino acid sequence of SEQ ID NO:521; (b) the light chain variable domain comprises the amino acid sequence of SEQ ID NO: 850 and the heavy chain variable domain comprises the amino acid sequence of SEQ ID NO:521; (c) the light chain variable domain comprises the amino acid sequence of SEQ ID NO: 334 and the heavy chain variable domain comprises the amino acid sequence of SEQ ID NO:522; (d) the light chain variable domain comprises the amino acid sequence of SEQ ID NO: 335 and the heavy chain variable domain comprises the amino acid sequence of SEQ ID NO:523; (e) the light chain variable domain comprises the amino acid sequence of SEQ ID NO: 336 and the heavy chain variable domain comprises the amino acid sequence of SEQ ID NO:524; (f) the light chain variable domain comprises the amino acid sequence of SEQ ID NO: 337 and the heavy chain variable domain comprises the amino acid sequence of SEQ ID NO:525; (g) the light chain variable domain comprises the amino acid sequence of SEQ ID NO: 338 and the heavy chain variable domain comprises the amino acid sequence of SEQ ID NO:526; (h) the light chain variable domain comprises the amino acid sequence of SEQ ID NO: 339 and the heavy chain variable domain comprises the amino acid sequence of SEQ ID NO:526; (i) the light chain variable domain comprises the amino acid sequence of SEQ ID NO: 340 and the heavy chain variable domain comprises the amino acid sequence of SEQ ID NO:527; (j) the light chain variable domain comprises the amino acid sequence of SEQ ID NO: 341 and the heavy chain variable domain comprises the amino acid sequence of SEQ ID NO:528; (k) the light chain variable domain comprises the amino acid sequence of SEQ ID NO: 342 and the heavy chain variable domain comprises the amino acid sequence of SEQ ID NO:529; (1) the light chain variable domain comprises the amino acid sequence of SEQ ID NO: 343 and the heavy chain variable domain comprises the amino acid sequence of SEQ ID NO:530; (m) the light chain variable domain comprises the amino acid sequence of SEQ ID NO: 843 and the heavy chain variable domain comprises the amino acid sequence of SEQ ID NO:845; (n) the light chain variable domain comprises the amino acid sequence of SEQ ID NO: 844 and the heavy chain variable domain comprises the amino acid sequence of SEQ ID NO:846; (o) the light chain variable domain comprises the amino acid sequence of SEQ ID NO:844 and the heavy chain variable domain comprises the amino acid sequence of SEQ ID NO:847; (p) the light chain variable domain comprises the amino acid sequence of SEQ ID NO: 219 and the heavy chain variable domain comprises the amino acid sequence of SEQ ID NO:399; (q) the light chain variable domain comprises the amino acid sequence of SEQ ID NO: 230 and the heavy chain variable domain comprises the amino acid sequence of SEQ ID NO:409; (r) the light chain variable domain comprises the amino acid sequence of SEQ ID NO: 252 and the heavy chain variable domain comprises the amino acid sequence of SEQ ID NO:419; (s) the light chain variable domain comprises the amino acid sequence of SEQ ID NO: 241 and the heavy chain variable domain comprises the amino acid sequence of SEQ ID NO:429; (t) the light chain variable domain comprises the amino acid sequence of SEQ ID NO: 849 and the heavy chain variable domain comprises the amino acid sequence of SEQ ID NO:429; (u) the light chain variable domain comprises the amino acid sequence of SEQ ID NO: 263 and the heavy chain variable domain comprises the amino acid sequence of SEQ ID NO:439; (v) the light chain variable domain comprises the amino acid sequence of SEQ ID NO: 274 and the heavy chain variable domain comprises the amino acid sequence of SEQ ID NO:449; (w) the light chain variable domain comprises the amino acid sequence of SEQ ID NO:285 and the heavy chain variable domain comprises the amino acid sequence of SEQ ID NO:459; (x) the light chain variable domain comprises the amino acid sequence of SEQ ID NO:286 and the heavy chain variable domain comprises the amino acid sequence of SEQ ID NO:460; (y) the light chain variable domain comprises the amino acid sequence of SEQ ID NO: 287 and the heavy chain variable domain comprises the amino acid sequence of SEQ ID NO:461; (z) the light chain variable domain comprises the amino acid sequence of SEQ ID NO: 298 and the heavy chain variable domain comprises the amino acid sequence of SEQ ID NO:429; (aa) the light chain variable domain comprises the amino acid sequence of SEQ ID NO:299 and the heavy chain variable domain comprises the amino acid sequence of SEQ ID NO:471; (bb) the light chain variable domain comprises the amino acid sequence of SEQ ID NO: 310 and the heavy chain variable domain comprises the amino acid sequence of SEQ ID NO:461; (cc) the light chain variable domain comprises the amino acid sequence of SEQ ID NO: 679 and the heavy chain variable domain comprises the amino acid sequence of SEQ ID NO:481; (dd) the light chain variable domain comprises the amino acid sequence of SEQ ID NO: 311 and the heavy chain variable domain comprises the amino acid sequence of SEQ ID NO:491; (ee) the light chain variable domain comprises the amino acid sequence of SEQ ID NO: 322 and the heavy chain variable domain comprises the amino acid sequence of SEQ ID NO:511; (ff) the light chain variable domain comprises the amino acid sequence of SEQ ID NO: 344 and the heavy chain variable domain comprises the amino acid sequence of SEQ ID NO:531; (gg) the light chain variable domain comprises the amino acid sequence of SEQ ID NO: 355 and the heavy chain variable domain comprises the amino acid sequence of SEQ ID NO:635; (hh) the light chain variable domain comprises the amino acid sequence of SEQ ID NO: 365 and the heavy chain variable domain comprises the amino acid sequence of SEQ ID NO:541; (ii) the light chain variable domain comprises the amino acid sequence of SEQ ID NO: 376 and the heavy chain variable domain comprises the amino acid sequence of SEQ ID NO:551; (jj) the light chain variable domain comprises the amino acid sequence of SEQ ID NO: 387 and the heavy chain variable domain comprises the amino acid sequence of SEQ ID NO:561; (kk) the light chain variable domain comprises the amino acid sequence of SEQ ID NO: 398 and the heavy chain variable domain comprises the amino acid sequence of SEQ ID NO:571; (11) the light chain variable domain comprises the amino acid sequence of SEQ ID NO: 724 and the heavy chain variable domain comprises the amino acid sequence of SEQ ID NO:731; (mm) the light chain variable domain comprises the amino acid sequence of SEQ ID NO: 809 and the heavy chain variable domain comprises the amino acid sequence of SEQ ID NO:731; (nn) the light chain variable domain comprises the amino acid sequence of SEQ ID NO: 725 and the heavy chain variable domain comprises the amino acid sequence of SEQ ID NO:732; (oo) the light chain variable domain comprises the amino acid sequence of SEQ ID NO: 726 and the heavy chain variable domain comprises the amino acid sequence of SEQ ID NO:731; (pp) the light chain variable domain comprises the amino acid sequence of SEQ ID NO: 726 and the heavy chain variable domain comprises the amino acid sequence of SEQ ID NO:817; (qq) the light chain variable domain comprises the amino acid sequence of SEQ ID NO: 727 and the heavy chain variable domain comprises the amino acid sequence of SEQ ID NO:731; (rr) the light chain variable domain comprises the amino acid sequence of SEQ ID NO: 728 and the heavy chain variable domain comprises the amino acid sequence of SEQ ID NO:733; (ss) the light chain variable domain comprises the amino acid sequence of SEQ ID NO:810 and the heavy chain variable domain comprises the amino acid sequence of SEQ ID NO:818; (tt) the light chain variable domain comprises the amino acid sequence of SEQ ID NO:811 and the heavy chain variable domain comprises the amino acid sequence of SEQ ID NO:733; (uu) the light chain variable domain comprises the amino acid sequence of SEQ ID NO:729 and the heavy chain variable domain comprises the amino acid sequence of SEQ ID NO:731; (vv) the light chain variable domain comprises the amino acid sequence of SEQ ID NO:812 and the heavy chain variable domain comprises the amino acid sequence of SEQ ID NO:819; (ww) the light chain variable domain comprises the amino acid sequence of SEQ ID NO:729 and the heavy chain variable domain comprises the amino acid sequence of SEQ ID NO:820; (xx) the light chain variable domain comprises the amino acid sequence of SEQ ID NO: 730 and the heavy chain variable domain comprises the amino acid sequence of SEQ ID NO:731; (yy) the light chain variable domain comprises the amino acid sequence of SEQ ID NO:813 and the heavy chain variable domain comprises the amino acid sequence of SEQ ID NO:731; (zz) the light chain variable domain comprises the amino acid sequence of SEQ ID NO:814 and the heavy chain variable domain comprises the amino acid sequence of SEQ ID NO:822; (aaa) the light chain variable domain comprises the amino acid sequence of SEQ ID NO:815 and the heavy chain variable domain comprises the amino acid sequence of SEQ ID NO:824; or (bbb) the light chain variable domain comprises the amino acid sequence of SEQ ID NO:816 and the heavy chain variable domain comprises the amino acid sequence of SEQ ID NO:825. In some embodiments that may be combined with any of the preceding embodiments, the antibody comprises a light chain variable domain of an antibody selected from the group consisting of: 3B10, 7B3, 8F8, 9F5, 9G1, 9G3, 11A8, 12F9, 7E9, 7F6, 8C3, 2C5, 3C5, 4C12, 7D9, 2F6, 3A7, 7E5, 11H5, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; and/or a heavy chain variable domain of an antibody selected from the group consisting of: 3B10, 7B3, 8F8, 9F5, 9G1, 9G3, 11A8, 12F9, 7E9, 7F6, 8C3, 2C5, 3C5, 4C12, 7D9, 2F6, 3A7, 77E5, 11H5, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2. In some embodiments that may be combined with any of the preceding embodiments, the anti-TREM2 antibody comprises a light chain variable domain and a heavy chain variable domain, wherein the light chain variable domain comprises HVR-L1, HVR-L2, HVR-L3, the heavy chain variable domain comprises HVR-H1, HVR-H2, and HVR-H3, and wherein the HVR-H3 comprises an amino acid sequence selected from the group consisting of SEQ ID NOs: 85-102, 588, 589, 709, 710, and 763-770, or an amino acid sequence with at least about 90% homology to an amino acid sequence selected from the group consisting of SEQ ID NOs: 85-102, 588, 589, 709, 710, and 763-770.

Other aspects of the present disclosure relate to an isolated (e.g., monoclonal) antibody that binds to a TREM2 protein, wherein the antibody binds to one or more amino acids within amino acid residues selected from the group consisting of: (i) amino acid residues 19-174 of SEQ ID NO: 1, or amino acid residues on a TREM2 protein corresponding to amino acid residues 19-174 of SEQ ID NO: 1; (ii) amino acid residues 29-112 of SEQ ID NO: 1, or amino acid residues on a TREM2 protein corresponding to amino acid residues 29-112 of SEQ ID NO: 1; (iii) amino acid residues 113-174 of SEQ ID NO: 1, or amino acid residues on a TREM2 protein corresponding to amino acid residues 113-174 of SEQ ID NO: 1; (iv) amino acid residues 35-49 of SEQ ID NO: 1, or amino acid residues on a TREM2 protein corresponding to amino acid residues 35-49 of SEQ ID NO: 1; (v) amino acid residues 35-49 and 140-150 of SEQ ID NO: 1, or amino acid residues on a TREM2 protein corresponding to amino acid residues 35-49 and 140-150 of SEQ ID NO: 1; (vi) amino acid residues 39-49 of SEQ ID NO: 1, or amino acid residues on a TREM2 protein corresponding to amino acid residues 39-49 of SEQ ID NO: 1; (vii) amino acid residues 39-49 and 63-77 of SEQ ID NO: 1, or amino acid residues on a TREM2 protein corresponding to amino acid residues 39-49 and 63-77 of SEQ ID NO: 1; (viii) amino acid residues 51-61 of SEQ ID NO: 1, or amino acid residues on a TREM2 protein corresponding to amino acid residues 51-61 of SEQ ID NO: 1; (ix) amino acid residues 55-62 of SEQ ID NO: 1, or amino acid residues on a TREM2 protein corresponding to amino acid residues 55-62 of SEQ ID NO: 1; (x) amino acid residues 55-62, 104-109, and 148-158 of SEQ ID NO: 1, or amino acid residues on a TREM2 protein corresponding to amino acid residues 55-62, 104-109, and 148-158 of SEQ ID NO: 1; (xi) amino acid residues 55-62, 104-109, and 160-166 of SEQ ID NO: 1, or amino acid residues on a TREM2 protein corresponding to amino acid residues 55-62, 104-109, and 160-166 of SEQ ID NO: 1; (xii) amino acid residues 55-65 of SEQ ID NO: 1, or amino acid residues on a TREM2 protein corresponding to amino acid residues 55-65 of SEQ ID NO: 1; (xiii) amino acid residues 55-65 and 124-134 of SEQ ID NO: 1, or amino acid residues on a TREM2 protein corresponding to amino acid residues 55-65 and 124-134 of SEQ ID NO: 1; (xiv) amino acid residues 63-73 of SEQ ID NO: 1, or amino acid residues on a TREM2 protein corresponding to amino acid residues 63-73 of SEQ ID NO: 1; (xv) amino acid residues 63-77 of SEQ ID NO: 1, or amino acid residues on a TREM2 protein corresponding to amino acid residues 63-77 of SEQ ID NO: 1; (xvi) amino acid residues 104-109 of SEQ ID NO: 1, or amino acid residues on a TREM2 protein corresponding to amino acid residues 104-109 of SEQ ID NO: 1; (xvii) amino acid residues 117-133 of SEQ ID NO: 1, or amino acid residues on a TREM2 protein corresponding to amino acid residues 117-133 of SEQ ID NO: 1; (xviii) amino acid residues 124-134 of SEQ ID NO: 1, or amino acid residues on a TREM2 protein corresponding to amino acid residues 124-134 of SEQ ID NO: 1; (xix) amino acid residues 137-146 of SEQ ID NO: 1, or amino acid residues on a TREM2 protein corresponding to amino acid residues 137-146 of SEQ ID NO: 1; (xx) amino acid residues 139-147 of SEQ ID NO: 1, or amino acid residues on a TREM2 protein corresponding to amino acid residues 139-147 of SEQ ID NO: 1; (xxi) amino acid residues 139-149 of SEQ ID NO: 1, or amino acid residues on a TREM2 protein corresponding to amino acid residues 139-149 of SEQ ID NO: 1; (xxii) amino acid residues 140-150 of SEQ ID NO: 1, or amino acid residues on a TREM2 protein corresponding to amino acid residues 140-150 of SEQ ID NO: 1; (xxiii) amino acid residues 140-146 of SEQ ID NO: 1, or amino acid residues on a TREM2 protein corresponding to amino acid residues 140-146 of SEQ ID NO: 1; (xxiv) amino acid residues 140-143 of SEQ ID NO: 1, or amino acid residues on a TREM2 protein corresponding to amino acid residues 140-143 of SEQ ID NO: 1; (xxv) amino acid residues 142-152 of SEQ ID NO: 1, or amino acid residues on a TREM2 protein corresponding to amino acid residues 142-152 of SEQ ID NO: 1; (xxvi) amino acid residues 146-154 of SEQ ID NO: 1, or amino acid residues on a TREM2 protein corresponding to amino acid residues 146-154 of SEQ ID NO: 1; (xxvii) amino acid residues 148-158 of SEQ ID NO: 1, or amino acid residues on a TREM2 protein corresponding to amino acid residues 148-158 of SEQ ID NO: 1; (xxviii) amino acid residues 149-157 of SEQ ID NO: 1, or amino acid residues on a TREM2 protein corresponding to amino acid residues 149-157 of SEQ ID NO: 1; (xxix) amino acid residues 149 and 150 of SEQ ID NO: 1, or amino acid residues on a TREM2 protein corresponding to amino acid residues 149 and 150 of SEQ ID NO: 1; (xxx) amino acid residues 151-155 of SEQ ID NO: 1, or amino acid residues on a TREM2 protein corresponding to amino acid residues 151-155 of SEQ ID NO: 1; (xxxi) amino acid residues 154-161 of SEQ ID NO: 1, or amino acid residues on a TREM2 protein corresponding to amino acid residues 154-161 of SEQ ID NO: 1; (xxxii) amino acid residues 156-170 of SEQ ID NO: 1, or amino acid residues on a TREM2 protein corresponding to amino acid residues 156-170 of SEQ ID NO: 1; (xxxiii) amino acid residues 160-166 of SEQ ID NO: 1, or amino acid residues on a TREM2 protein corresponding to amino acid residues 160-166 of SEQ ID NO: 1; and (xxxiv) amino acid residues 162-165 of SEQ ID NO: 1, or amino acid residues on a TREM2 protein corresponding to amino acid residues 162-165 of SEQ ID NO: 1. In some embodiments, the antibody induces one or more TREM2 activities and enhances one or more TREM2 activities induced by binding of one or more TREM2 ligands to the TREM2 protein. In some embodiments, the antibody further binds to one or more amino acid residues selected from the group consisting of: (i) amino acid residue Arg47 or Asp87 of SEQ ID NO: 1; (ii) amino acid residues 40-44 of SEQ ID NO: 1; (iii) amino acid residues 67-76 of SEQ ID NO: 1; and (iv) amino acid residues 114-118 of SEQ ID NO: 1.

Other aspects of the present disclosure relate to an isolated (e.g., monoclonal) antibody that binds to a TREM2 protein, wherein the antibody binds to one or more amino acid residues selected from the group consisting of K42, H43, W44, G45, H67, R77, T88, H114, E117, E151, D152, H154, and E156 of SEQ ID NO: 1, or one or more amino acid residues on a mammalian TREM2 protein corresponding to an amino acid residue selected from the group consisting of K42, H43, W44, G45, H67, R77, T88, H114, E117, E151, D152, H154, and E156 of SEQ ID NO: 1. In some embodiments, the antibody binds to one or more amino acid residues selected from the group consisting of E151, D152, H154, and E156 of SEQ ID NO: 1, or one or more amino acid residues on a mammalian TREM2 protein corresponding to an amino acid residue selected from the group consisting of E151, D152, H154, and E156 of SEQ ID NO: 1. Other aspects of the present disclosure relate to an isolated (e.g., monoclonal) antibody that binds to a TREM2 protein, wherein the antibody binds to one or more amino acid residues selected from the group consisting of E151, D152, H154, and E156 of SEQ ID NO: 1, or one or more amino acid residues on a mammalian TREM2 protein corresponding to an amino acid residue selected from the group consisting of E151, D152, H154, and E156 of SEQ ID NO: 1.

Other aspects of the present disclosure relate to an isolated (e.g., monoclonal) antibody that binds to a TREM2 protein, wherein the antibody competes with one or more antibodies selected from the group consisting of 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, 44B4v2, and any combination thereof for binding to TREM2.

Other aspects of the present disclosure relate to an isolated (e.g., monoclonal) antibody that binds to a TREM2 protein, wherein the antibody comprises a light chain variable domain and a heavy chain variable domain, wherein the light chain variable domain, or the heavy chain variable domain, or both comprise at least one, two, three, four, five, or six HVRs selected from HVR-L1, HVR-L2, HVR-L3, HVR-H1, HVR-H2, and HVR-H3 of an antibody selected from the group consisting of: 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2. In some embodiments: (a) the HVR-L1 comprises an amino acid sequence selected from the group consisting of SEQ ID NOs: 9-23, 581, 690-694, 734-738, and 826-828; (b) the HVR-L2 comprises an amino acid sequence selected from the group consisting of SEQ ID NOs: 24-33, 695-697, and 739-743; (c) the HVR-L3 comprises an amino acid sequence selected from the group consisting of SEQ ID NOs: 34-47, 582, 583, 698-702, and 744-746; (d) the HVR-H1 comprises an amino acid sequence selected from the group consisting of SEQ ID NOs: 48-65, 584, 703-705, 747-754, and 829-835; (e) the HVR-H2 comprises an amino acid sequence selected from the group consisting of SEQ ID NOs: 66-84, 585-587, 706-708, 755-762, 836-842, and 888; or (f) the HVR-H3 comprises an amino acid sequence selected from the group consisting of SEQ ID NOs: 85-102, 588, 589, 709, 710, and 763-770. In some embodiments: (a) the HVR-L1 comprises the amino acid sequence of SEQ ID NO: 9, the HVR-L2 comprises the amino acid sequence of SEQ ID NO: 24, the HVR-L3 comprises the amino acid sequence of SEQ ID NO: 34, the HVR-H1 comprises the amino acid sequence of SEQ ID NO: 48, the HVR-H2 comprises the amino acid sequence of SEQ ID NO: 66, and the HVR-H3 comprises the amino acid sequence of SEQ ID NO: 85; (b) the HVR-L1 comprises the amino acid sequence of SEQ ID NO: 9, the HVR-L2 comprises the amino acid sequence of SEQ ID NO: 24, the HVR-L3 comprises the amino acid sequence of SEQ ID NO: 34, the HVR-H1 comprises the amino acid sequence of SEQ ID NO: 48, the HVR-H2 comprises the amino acid sequence of SEQ ID NO: 66, and the HVR-H3 comprises the amino acid sequence of SEQ ID NO: 85; (c) the HVR-L1 comprises the amino acid sequence of SEQ ID NO: 10, the HVR-L2 comprises the amino acid sequence of SEQ ID NO: 25, the HVR-L3 comprises the amino acid sequence of SEQ ID NO: 35, the HVR-H1 comprises the amino acid sequence of SEQ ID NO: 49, the HVR-H2 comprises the amino acid sequence of SEQ ID NO: 67, and the HVR-H3 comprises the amino acid sequence of SEQ ID NO: 86; (d) the HVR-L1 comprises the amino acid sequence of SEQ ID NO: 12, the HVR-L2 comprises the amino acid sequence of SEQ ID NO: 26, the HVR-L3 comprises the amino acid sequence of SEQ ID NO: 37, the HVR-H1 comprises the amino acid sequence of SEQ ID NO: 50, the HVR-H2 comprises the amino acid sequence of SEQ ID NO: 68, and the HVR-H3 comprises the amino acid sequence of SEQ ID NO: 87; (e) the HVR-L1 comprises the amino acid sequence of SEQ ID NO: 11, the HVR-L2 comprises the amino acid sequence of SEQ ID NO: 26, the HVR-L3 comprises the amino acid sequence of SEQ ID NO: 36, the HVR-H1 comprises the amino acid sequence of SEQ ID NO: 51, the HVR-H2 comprises the amino acid sequence of SEQ ID NO: 69, and the HVR-H3 comprises the amino acid sequence of SEQ ID NO: 88; (f) the HVR-L1 comprises the amino acid sequence of SEQ ID NO: 13, the HVR-L2 comprises the amino acid sequence of SEQ ID NO: 27, the HVR-L3 comprises the amino acid sequence of SEQ ID NO: 38, the HVR-H1 comprises the amino acid sequence of SEQ ID NO: 52, the HVR-H2 comprises the amino acid sequence of SEQ ID NO: 70, and the HVR-H3 comprises the amino acid sequence of SEQ ID NO: 89; (g) the HVR-L1 comprises the amino acid sequence of SEQ ID NO: 14, the HVR-L2 comprises the amino acid sequence of SEQ ID NO: 28, the HVR-L3 comprises the amino acid sequence of SEQ ID NO: 39, the HVR-H1 comprises the amino acid sequence of SEQ ID NO: 53, the HVR-H2 comprises the amino acid sequence of SEQ ID NO: 71, and the HVR-H3 comprises the amino acid sequence of SEQ ID NO: 90; (h) the HVR-L1 comprises the amino acid sequence of SEQ ID NO: 13, the HVR-L2 comprises the amino acid sequence of SEQ ID NO: 27, the HVR-L3 comprises the amino acid sequence of SEQ ID NO: 38, the HVR-H1 comprises the amino acid sequence of SEQ ID NO: 52, the HVR-H2 comprises the amino acid sequence of SEQ ID NO: 70, and the HVR-H3 comprises the amino acid sequence of SEQ ID NO: 89; (i) the HVR-L1 comprises the amino acid sequence of SEQ ID NO: 13, the HVR-L2 comprises the amino acid sequence of SEQ ID NO: 27, the HVR-L3 comprises the amino acid sequence of SEQ ID NO: 38, the HVR-H1 comprises the amino acid sequence of SEQ ID NO: 52, the HVR-H2 comprises the amino acid sequence of SEQ ID NO: 70, and the HVR-H3 comprises the amino acid sequence of SEQ ID NO: 89; (j) the HVR-L1 comprises the amino acid sequence of SEQ ID NO: 15, the HVR-L2 comprises the amino acid sequence of SEQ ID NO: 28, the HVR-L3 comprises the amino acid sequence of SEQ ID NO: 40, the HVR-H1 comprises the amino acid sequence of SEQ ID NO: 54, the HVR-H2 comprises the amino acid sequence of SEQ ID NO: 72, and the HVR-H3 comprises the amino acid sequence of SEQ ID NO: 91; (k) the HVR-L1 comprises the amino acid sequence of SEQ ID NO: 11, the HVR-L2 comprises the amino acid sequence of SEQ ID NO: 26, the HVR-L3 comprises the amino acid sequence of SEQ ID NO: 36, the HVR-H1 comprises the amino acid sequence of SEQ ID NO: 51, the HVR-H2 comprises the amino acid sequence of SEQ ID NO: 69, and the HVR-H3 comprises the amino acid sequence of SEQ ID NO: 88; (1) the HVR-L1 comprises the amino acid sequence of SEQ ID NO: 16, the HVR-L2 comprises the amino acid sequence of SEQ ID NO: 29, the HVR-L3 comprises the amino acid sequence of SEQ ID NO: 35, the HVR-H1 comprises the amino acid sequence of SEQ ID NO: 55, the HVR-H2 comprises the amino acid sequence of SEQ ID NO: 73, and the HVR-H3 comprises the amino acid sequence of SEQ ID NO: 92; (m) the HVR-L1 comprises the amino acid sequence of SEQ ID NO: 15, the HVR-L2 comprises the amino acid sequence of SEQ ID NO: 28, the HVR-L3 comprises the amino acid sequence of SEQ ID NO: 40, the HVR-H1 comprises the amino acid sequence of SEQ ID NO: 54, the HVR-H2 comprises the amino acid sequence of SEQ ID NO: 72, and the HVR-H3 comprises the amino acid sequence of SEQ ID NO: 91; (n) the HVR-L1 comprises the amino acid sequence of SEQ ID NO: 581, the HVR-L2 comprises the amino acid sequence of SEQ ID NO: 29, the HVR-L3 comprises the amino acid sequence of SEQ ID NO: 582, the HVR-H1 comprises the amino acid sequence of SEQ ID NO: 56, the HVR-H2 comprises the amino acid sequence of SEQ ID NO: 74, and the HVR-H3 comprises the amino acid sequence of SEQ ID NO: 93; (o) the HVR-L1 comprises the amino acid sequence of SEQ ID NO: 17, the HVR-L2 comprises the amino acid sequence of SEQ ID NO: 30, the HVR-L3 comprises the amino acid sequence of SEQ ID NO: 41, the HVR-H1 comprises the amino acid sequence of SEQ ID NO: 57, the HVR-H2 comprises the amino acid sequence of SEQ ID NO: 75, and the HVR-H3 comprises the amino acid sequence of SEQ ID NO: 94; (p) the HVR-H1 comprises the amino acid sequence of SEQ ID NO: 58, the HVR-H2 comprises the amino acid sequence of SEQ ID NO: 76, and the HVR-H3 comprises the amino acid sequence of SEQ ID NO: 95; (q) the HVR-L1 comprises the amino acid sequence of SEQ ID NO: 18, the HVR-L2 comprises the amino acid sequence of SEQ ID NO: 31, the HVR-L3 comprises the amino acid sequence of SEQ ID NO: 42, the HVR-H1 comprises the amino acid sequence of SEQ ID NO: 59, the HVR-H2 comprises the amino acid sequence of SEQ ID NO: 77, and the HVR-H3 comprises the amino acid sequence of SEQ ID NO: 96; (r) the HVR-L1 comprises the amino acid sequence of SEQ ID NO: 19, the HVR-L2 comprises the amino acid sequence of SEQ ID NO: 28, the HVR-L3 comprises the amino acid sequence of SEQ ID NO: 43, the HVR-H1 comprises the amino acid sequence of SEQ ID NO: 60, the HVR-H2 comprises the amino acid sequence of SEQ ID NO: 78, and the HVR-H3 comprises the amino acid sequence of SEQ ID NO: 97; (s) the HVR-L1 comprises the amino acid sequence of SEQ ID NO: 20, the HVR-L2 comprises the amino acid sequence of SEQ ID NO: 28, the HVR-L3 comprises the amino acid sequence of SEQ ID NO: 44, the HVR-H1 comprises the amino acid sequence of SEQ ID NO: 61, the HVR-H2 comprises the amino acid sequence of SEQ ID NO: 79, and the HVR-H3 comprises the amino acid sequence of SEQ ID NO: 98; (t) the HVR-L1 comprises the amino acid sequence of SEQ ID NO: 21, the HVR-L2 comprises the amino acid sequence of SEQ ID NO: 32, the HVR-L3 comprises the amino acid sequence of SEQ ID NO: 45, the HVR-H1 comprises the amino acid sequence of SEQ ID NO: 62, the HVR-H2 comprises the amino acid sequence of SEQ ID NO: 80, and the HVR-H3 comprises the amino acid sequence of SEQ ID NO: 99; (u) the HVR-L1 comprises the amino acid sequence of SEQ ID NO: 15, the HVR-L2 comprises the amino acid sequence of SEQ ID NO: 33, the HVR-L3 comprises the amino acid sequence of SEQ ID NO: 40, the HVR-H1 comprises the amino acid sequence of SEQ ID NO: 54, the HVR-H2 comprises the amino acid sequence of SEQ ID NO: 81, and the HVR-H3 comprises the amino acid sequence of SEQ ID NO: 91; (v) the HVR-L1 comprises the amino acid sequence of SEQ ID NO: 22, the HVR-L2 comprises the amino acid sequence of SEQ ID NO: 29, the HVR-L3 comprises the amino acid sequence of SEQ ID NO: 46, the HVR-H1 comprises the amino acid sequence of SEQ ID NO: 63, the HVR-H2 comprises the amino acid sequence of SEQ ID NO: 82, and the HVR-H3 comprises the amino acid sequence of SEQ ID NO: 100; (w) the HVR-L1 comprises the amino acid sequence of SEQ ID NO: 23, the HVR-L2 comprises the amino acid sequence of SEQ ID NO: 29, the HVR-L3 comprises the amino acid sequence of SEQ ID NO: 47, the HVR-H1 comprises the amino acid sequence of SEQ ID NO: 64, the HVR-H2 comprises the amino acid sequence of SEQ ID NO: 83, and the HVR-H3 comprises the amino acid sequence of SEQ ID NO: 101; (x) the HVR-L1 comprises the amino acid sequence of SEQ ID NO: 16, the HVR-L2 comprises the amino acid sequence of SEQ ID NO: 29, the HVR-L3 comprises the amino acid sequence of SEQ ID NO: 35, the HVR-H1 comprises the amino acid sequence of SEQ ID NO: 65, the HVR-H2 comprises the amino acid sequence of SEQ ID NO: 84, and the HVR-H3 comprises the amino acid sequence of SEQ ID NO: 102; (y) the HVR-L1 comprises the amino acid sequence of SEQ ID NO: 581, the HVR-L2 comprises the amino acid sequence of SEQ ID NO: 29, the HVR-L3 comprises the amino acid sequence of SEQ ID NO: 582, the HVR-H1 comprises the amino acid sequence of SEQ ID NO: 56, the HVR-H2 comprises the amino acid sequence of SEQ ID NO: 585, and the HVR-H3 comprises the amino acid sequence of SEQ ID NO: 588; (z) the HVR-L1 comprises the amino acid sequence of SEQ ID NO: 10, the HVR-L2 comprises the amino acid sequence of SEQ ID NO: 29, the HVR-L3 comprises the amino acid sequence of SEQ ID NO: 35, the HVR-H1 comprises the amino acid sequence of SEQ ID NO: 49, the HVR-H2 comprises the amino acid sequence of SEQ ID NO: 586, and the HVR-H3 comprises the amino acid sequence of SEQ ID NO: 86; or (aa) the HVR-L1 comprises the amino acid sequence of SEQ ID NO: 14, the HVR-L2 comprises the amino acid sequence of SEQ ID NO: 28, the HVR-L3 comprises the amino acid sequence of SEQ ID NO: 583, the HVR-H1 comprises the amino acid sequence of SEQ ID NO: 584, the HVR-H2 comprises the amino acid sequence of SEQ ID NO: 587, and the HVR-H3 comprises the amino acid sequence of SEQ ID NO: 589. In some embodiments, the light chain variable domain comprises: (a) an HVR-L1 comprising an amino acid sequence selected from the group consisting of SEQ ID NOs: 9-23, 581, 690-694, 734-738, and 826-828, or an amino acid sequence with at least about 90% homology to an amino acid sequence selected from the group consisting of SEQ ID NOs: 9-23, 581, 690-694, 734-738, and 826-828; (b) an HVR-L2 comprising an amino acid sequence selected from the group consisting of SEQ ID NOs: 24-33, 695-697, and 739-743, or an amino acid sequence with at least about 90% homology to an amino acid sequence selected from the group consisting of SEQ ID NOs: 24-33, 695-697, and 739-743; and (c) an HVR-L3 comprising an amino acid sequence selected from the group consisting of SEQ ID NOs: 34-47, 582, 583, 698-702, and 744-746, or an amino acid sequence with at least about 90% homology to an amino acid sequence selected from the group consisting of SEQ ID NOs: 34-47, 582, 583, 698-702, and 744-746; and wherein the heavy chain variable domain comprises: (a) an HVR-H1 comprising an amino acid sequence selected from the group consisting of SEQ ID NOs: 48-65, 584, 703-705, 747-754, and 829-835, or an amino acid sequence with at least about 90% homology to an amino acid sequence selected from the group consisting of SEQ ID NOs: 48-65, 584, 703-705, 747-754, and 829-835; (b) an HVR-H2 comprising an amino acid sequence selected from the group consisting of SEQ ID NOs: 66-84, 585-587, 706-708, 755-762, 836-842, and 888, or an amino acid sequence with at least about 90% homology to an amino acid sequence selected from the group consisting of SEQ ID NOs: 66-84, 585-587, 706-708, 755-762, 836-842, and 888; and (c) an HVR-H3 comprising an amino acid sequence selected from the group consisting of SEQ ID NOs: 85-102, 588, 589, 709, 710, and 763-770, or an amino acid sequence with at least about 90% homology to an amino acid sequence selected from the group consisting of SEQ ID NOs: 85-102, 588, 589, 709, 710, and 763-770. In some embodiments, the anti-TREM2 antibody comprises a light chain variable domain and a heavy chain variable domain, wherein the light chain variable domain comprises HVR-L1, HVR-L2, HVR-L3, the heavy chain variable domain comprises HVR-H1, HVR-H2, and HVR-H3, and wherein the HVR-H3 comprises an amino acid sequence selected from the group consisting of SEQ ID NOs: 85-102, 588, 589, 709, 710, and 763-770, or an amino acid sequence with at least about 90% homology to an amino acid sequence selected from the group consisting of SEQ ID NOs: 85-102, 588, 589, 709, 710, and 763-770. In some embodiments, the antibody comprises a light chain variable domain comprising an amino acid sequence selected from the group consisting of SEQ ID NOs: 219-398, 602-634, 679-689, 724-730, 809-816, 821, 843, 844, 849, and 850; and/or a heavy chain variable domain comprising an amino acid sequence selected from the group consisting of SEQ ID NOs: 399-580, 635-678, 731-733, 817-820, 822-825, and 845-847. hi some embodiments, the antibody comprises a light chain variable domain and a heavy chain variable domain, wherein: (a) the light chain variable domain comprises the amino acid sequence of SEQ ID NO: 333 and the heavy chain variable domain comprises the amino acid sequence of SEQ ID NO:521; (b) the light chain variable domain comprises the amino acid sequence of SEQ ID NO: 850 and the heavy chain variable domain comprises the amino acid sequence of SEQ ID NO:521; (c) the light chain variable domain comprises the amino acid sequence of SEQ ID NO: 334 and the heavy chain variable domain comprises the amino acid sequence of SEQ ID NO:522; (d) the light chain variable domain comprises the amino acid sequence of SEQ ID NO: 335 and the heavy chain variable domain comprises the amino acid sequence of SEQ ID NO:523; (e) the light chain variable domain comprises the amino acid sequence of SEQ ID NO: 336 and the heavy chain variable domain comprises the amino acid sequence of SEQ ID NO:524; (f) the light chain variable domain comprises the amino acid sequence of SEQ ID NO: 337 and the heavy chain variable domain comprises the amino acid sequence of SEQ ID NO:525; (g) the light chain variable domain comprises the amino acid sequence of SEQ ID NO: 338 and the heavy chain variable domain comprises the amino acid sequence of SEQ ID NO:526; (h) the light chain variable domain comprises the amino acid sequence of SEQ ID NO: 339 and the heavy chain variable domain comprises the amino acid sequence of SEQ ID NO:526; (i) the light chain variable domain comprises the amino acid sequence of SEQ ID NO: 340 and the heavy chain variable domain comprises the amino acid sequence of SEQ ID NO:527; (j) the light chain variable domain comprises the amino acid sequence of SEQ ID NO: 341 and the heavy chain variable domain comprises the amino acid sequence of SEQ ID NO:528; (k) the light chain variable domain comprises the amino acid sequence of SEQ ID NO: 342 and the heavy chain variable domain comprises the amino acid sequence of SEQ ID NO:529; (1) the light chain variable domain comprises the amino acid sequence of SEQ ID NO: 343 and the heavy chain variable domain comprises the amino acid sequence of SEQ ID NO:530; (m) the light chain variable domain comprises the amino acid sequence of SEQ ID NO: 843 and the heavy chain variable domain comprises the amino acid sequence of SEQ ID NO:845; (n) the light chain variable domain comprises the amino acid sequence of SEQ ID NO: 844 and the heavy chain variable domain comprises the amino acid sequence of SEQ ID NO:846; (o) the light chain variable domain comprises the amino acid sequence of SEQ ID NO:844 and the heavy chain variable domain comprises the amino acid sequence of SEQ ID NO:847; (p) the light chain variable domain comprises the amino acid sequence of SEQ ID NO: 219 and the heavy chain variable domain comprises the amino acid sequence of SEQ ID NO:399; (q) the light chain variable domain comprises the amino acid sequence of SEQ ID NO: 230 and the heavy chain variable domain comprises the amino acid sequence of SEQ ID NO:409; (r) the light chain variable domain comprises the amino acid sequence of SEQ ID NO: 252 and the heavy chain variable domain comprises the amino acid sequence of SEQ ID NO:419; (s) the light chain variable domain comprises the amino acid sequence of SEQ ID NO: 241 and the heavy chain variable domain comprises the amino acid sequence of SEQ ID NO:429; (t) the light chain variable domain comprises the amino acid sequence of SEQ ID NO: 849 and the heavy chain variable domain comprises the amino acid sequence of SEQ ID NO:429; (u) the light chain variable domain comprises the amino acid sequence of SEQ ID NO: 263 and the heavy chain variable domain comprises the amino acid sequence of SEQ ID NO:439; (v) the light chain variable domain comprises the amino acid sequence of SEQ ID NO: 274 and the heavy chain variable domain comprises the amino acid sequence of SEQ ID NO:449; (w) the light chain variable domain comprises the amino acid sequence of SEQ ID NO:285 and the heavy chain variable domain comprises the amino acid sequence of SEQ ID NO:459; (x) the light chain variable domain comprises the amino acid sequence of SEQ ID NO:286 and the heavy chain variable domain comprises the amino acid sequence of SEQ ID NO:460; (y) the light chain variable domain comprises the amino acid sequence of SEQ ID NO: 287 and the heavy chain variable domain comprises the amino acid sequence of SEQ ID NO:461; (z) the light chain variable domain comprises the amino acid sequence of SEQ ID NO: 298 and the heavy chain variable domain comprises the amino acid sequence of SEQ ID NO:429; (aa) the light chain variable domain comprises the amino acid sequence of SEQ ID NO:299 and the heavy chain variable domain comprises the amino acid sequence of SEQ ID NO:471; (bb) the light chain variable domain comprises the amino acid sequence of SEQ ID NO: 310 and the heavy chain variable domain comprises the amino acid sequence of SEQ ID NO:461; (cc) the light chain variable domain comprises the amino acid sequence of SEQ ID NO: 679 and the heavy chain variable domain comprises the amino acid sequence of SEQ ID NO:481; (dd) the light chain variable domain comprises the amino acid sequence of SEQ ID NO: 311 and the heavy chain variable domain comprises the amino acid sequence of SEQ ID NO:491; (ee) the light chain variable domain comprises the amino acid sequence of SEQ ID NO: 322 and the heavy chain variable domain comprises the amino acid sequence of SEQ ID NO:511; (ff) the light chain variable domain comprises the amino acid sequence of SEQ ID NO: 344 and the heavy chain variable domain comprises the amino acid sequence of SEQ ID NO:531; (gg) the light chain variable domain comprises the amino acid sequence of SEQ ID NO: 355 and the heavy chain variable domain comprises the amino acid sequence of SEQ ID NO:635; (hh) the light chain variable domain comprises the amino acid sequence of SEQ ID NO: 365 and the heavy chain variable domain comprises the amino acid sequence of SEQ ID NO:541; (ii) the light chain variable domain comprises the amino acid sequence of SEQ ID NO: 376 and the heavy chain variable domain comprises the amino acid sequence of SEQ ID NO:551; (jj) the light chain variable domain comprises the amino acid sequence of SEQ ID NO: 387 and the heavy chain variable domain comprises the amino acid sequence of SEQ ID NO:561; (kk) the light chain variable domain comprises the amino acid sequence of SEQ ID NO: 398 and the heavy chain variable domain comprises the amino acid sequence of SEQ ID NO:571; (11) the light chain variable domain comprises the amino acid sequence of SEQ ID NO: 724 and the heavy chain variable domain comprises the amino acid sequence of SEQ ID NO:731; (mm) the light chain variable domain comprises the amino acid sequence of SEQ ID NO: 809 and the heavy chain variable domain comprises the amino acid sequence of SEQ ID NO:731; (nn) the light chain variable domain comprises the amino acid sequence of SEQ ID NO: 725 and the heavy chain variable domain comprises the amino acid sequence of SEQ ID NO:732; (oo) the light chain variable domain comprises the amino acid sequence of SEQ ID NO: 726 and the heavy chain variable domain comprises the amino acid sequence of SEQ ID NO:731; (pp) the light chain variable domain comprises the amino acid sequence of SEQ ID NO: 726 and the heavy chain variable domain comprises the amino acid sequence of SEQ ID NO:817; (qq) the light chain variable domain comprises the amino acid sequence of SEQ ID NO: 727 and the heavy chain variable domain comprises the amino acid sequence of SEQ ID NO:731; (rr) the light chain variable domain comprises the amino acid sequence of SEQ ID NO: 728 and the heavy chain variable domain comprises the amino acid sequence of SEQ ID NO:733; (ss) the light chain variable domain comprises the amino acid sequence of SEQ ID NO:810 and the heavy chain variable domain comprises the amino acid sequence of SEQ ID NO:818; (tt) the light chain variable domain comprises the amino acid sequence of SEQ ID NO:811 and the heavy chain variable domain comprises the amino acid sequence of SEQ ID NO:733; (uu) the light chain variable domain comprises the amino acid sequence of SEQ ID NO:729 and the heavy chain variable domain comprises the amino acid sequence of SEQ ID NO:731; (vv) the light chain variable domain comprises the amino acid sequence of SEQ ID NO:812 and the heavy chain variable domain comprises the amino acid sequence of SEQ ID NO:819; (ww) the light chain variable domain comprises the amino acid sequence of SEQ ID NO:729 and the heavy chain variable domain comprises the amino acid sequence of SEQ ID NO:820; (xx) the light chain variable domain comprises the amino acid sequence of SEQ ID NO: 730 and the heavy chain variable domain comprises the amino acid sequence of SEQ ID NO:731; (yy) the light chain variable domain comprises the amino acid sequence of SEQ ID NO:813 and the heavy chain variable domain comprises the amino acid sequence of SEQ ID NO:731; (zz) the light chain variable domain comprises the amino acid sequence of SEQ ID NO:814 and the heavy chain variable domain comprises the amino acid sequence of SEQ ID NO:822; (aaa) the light chain variable domain comprises the amino acid sequence of SEQ ID NO:815 and the heavy chain variable domain comprises the amino acid sequence of SEQ ID NO:824; or (bbb) the light chain variable domain comprises the amino acid sequence of SEQ ID NO:816 and the heavy chain variable domain comprises the amino acid sequence of SEQ ID NO:825.

Other aspects of the present disclosure relate to an isolated (e.g., monoclonal) antibody that binds to a TREM2 protein, wherein the antibody comprises a light chain variable domain comprising an amino acid sequence selected from the group consisting of SEQ ID NOs: 219-398, 602-634, 679-689, 724-730, 809-816, 821, 843, 844, 849, and 850; and/or a heavy chain variable domain comprising an amino acid sequence selected from the group consisting of SEQ ID NOs: 399-580, 635-678, 731-733, and 817-820, 822-825, and 845-847. In some embodiments, the antibody comprises a light chain variable domain comprising the amino acid sequence of SEQ ID NO:843 and a heavy chain variable domain comprising the amino acid sequence of SEQ ID NO:845. In some embodiments, the antibody comprises a light chain variable domain comprising the amino acid sequence of SEQ ID NO:843 and a heavy chain variable domain comprising the amino acid sequence of SEQ ID NO:846. In some embodiments, the antibody comprises a light chain variable domain comprising the amino acid sequence of SEQ ID NO:843 and a heavy chain variable domain comprising the amino acid sequence of SEQ ID NO:847. In some embodiments, the antibody comprises a light chain variable domain comprising the amino acid sequence of SEQ ID NO:844 and a heavy chain variable domain comprising the amino acid sequence of SEQ ID NO:847. In some embodiments, the antibody comprises a light chain variable domain and a heavy chain variable domain, wherein: (a) the light chain variable domain comprises the amino acid sequence of SEQ ID NO: 333 and the heavy chain variable domain comprises the amino acid sequence of SEQ ID NO:521; (b) the light chain variable domain comprises the amino acid sequence of SEQ ID NO: 850 and the heavy chain variable domain comprises the amino acid sequence of SEQ ID NO:521; (c) the light chain variable domain comprises the amino acid sequence of SEQ ID NO: 334 and the heavy chain variable domain comprises the amino acid sequence of SEQ ID NO:522; (d) the light chain variable domain comprises the amino acid sequence of SEQ ID NO: 335 and the heavy chain variable domain comprises the amino acid sequence of SEQ ID NO:523; (e) the light chain variable domain comprises the amino acid sequence of SEQ ID NO: 336 and the heavy chain variable domain comprises the amino acid sequence of SEQ ID NO:524; (f) the light chain variable domain comprises the amino acid sequence of SEQ ID NO: 337 and the heavy chain variable domain comprises the amino acid sequence of SEQ ID NO:525; (g) the light chain variable domain comprises the amino acid sequence of SEQ ID NO: 338 and the heavy chain variable domain comprises the amino acid sequence of SEQ ID NO:526; (h) the light chain variable domain comprises the amino acid sequence of SEQ ID NO: 339 and the heavy chain variable domain comprises the amino acid sequence of SEQ ID NO:526; (i) the light chain variable domain comprises the amino acid sequence of SEQ ID NO: 340 and the heavy chain variable domain comprises the amino acid sequence of SEQ ID NO:527; (j) the light chain variable domain comprises the amino acid sequence of SEQ ID NO: 341 and the heavy chain variable domain comprises the amino acid sequence of SEQ ID NO:528; (k) the light chain variable domain comprises the amino acid sequence of SEQ ID NO: 342 and the heavy chain variable domain comprises the amino acid sequence of SEQ ID NO:529; (1) the light chain variable domain comprises the amino acid sequence of SEQ ID NO: 343 and the heavy chain variable domain comprises the amino acid sequence of SEQ ID NO:530; (m) the light chain variable domain comprises the amino acid sequence of SEQ ID NO: 843 and the heavy chain variable domain comprises the amino acid sequence of SEQ ID NO:845; (n) the light chain variable domain comprises the amino acid sequence of SEQ ID NO: 844 and the heavy chain variable domain comprises the amino acid sequence of SEQ ID NO:846; (o) the light chain variable domain comprises the amino acid sequence of SEQ ID NO:844 and the heavy chain variable domain comprises the amino acid sequence of SEQ ID NO:847; (p) the light chain variable domain comprises the amino acid sequence of SEQ ID NO: 219 and the heavy chain variable domain comprises the amino acid sequence of SEQ ID NO:399; (q) the light chain variable domain comprises the amino acid sequence of SEQ ID NO: 230 and the heavy chain variable domain comprises the amino acid sequence of SEQ ID NO:409; (r) the light chain variable domain comprises the amino acid sequence of SEQ ID NO: 252 and the heavy chain variable domain comprises the amino acid sequence of SEQ ID NO:419; (s) the light chain variable domain comprises the amino acid sequence of SEQ ID NO: 241 and the heavy chain variable domain comprises the amino acid sequence of SEQ ID NO:429; (t) the light chain variable domain comprises the amino acid sequence of SEQ ID NO: 849 and the heavy chain variable domain comprises the amino acid sequence of SEQ ID NO:429; (u) the light chain variable domain comprises the amino acid sequence of SEQ ID NO: 263 and the heavy chain variable domain comprises the amino acid sequence of SEQ ID NO:439; (v) the light chain variable domain comprises the amino acid sequence of SEQ ID NO: 274 and the heavy chain variable domain comprises the amino acid sequence of SEQ ID NO:449; (w) the light chain variable domain comprises the amino acid sequence of SEQ ID NO:285 and the heavy chain variable domain comprises the amino acid sequence of SEQ ID NO:459; (x) the light chain variable domain comprises the amino acid sequence of SEQ ID NO:286 and the heavy chain variable domain comprises the amino acid sequence of SEQ ID NO:460; (y) the light chain variable domain comprises the amino acid sequence of SEQ ID NO: 287 and the heavy chain variable domain comprises the amino acid sequence of SEQ ID NO:461; (z) the light chain variable domain comprises the amino acid sequence of SEQ ID NO: 298 and the heavy chain variable domain comprises the amino acid sequence of SEQ ID NO:429; (aa) the light chain variable domain comprises the amino acid sequence of SEQ ID NO:299 and the heavy chain variable domain comprises the amino acid sequence of SEQ ID NO:471; (bb) the light chain variable domain comprises the amino acid sequence of SEQ ID NO: 310 and the heavy chain variable domain comprises the amino acid sequence of SEQ ID NO:461; (cc) the light chain variable domain comprises the amino acid sequence of SEQ ID NO: 679 and the heavy chain variable domain comprises the amino acid sequence of SEQ ID NO:481; (dd) the light chain variable domain comprises the amino acid sequence of SEQ ID NO: 311 and the heavy chain variable domain comprises the amino acid sequence of SEQ ID NO:491; (ee) the light chain variable domain comprises the amino acid sequence of SEQ ID NO: 322 and the heavy chain variable domain comprises the amino acid sequence of SEQ ID NO:511; (ff) the light chain variable domain comprises the amino acid sequence of SEQ ID NO: 344 and the heavy chain variable domain comprises the amino acid sequence of SEQ ID NO:531; (gg) the light chain variable domain comprises the amino acid sequence of SEQ ID NO: 355 and the heavy chain variable domain comprises the amino acid sequence of SEQ ID NO:635; (hh) the light chain variable domain comprises the amino acid sequence of SEQ ID NO: 365 and the heavy chain variable domain comprises the amino acid sequence of SEQ ID NO:541; (ii) the light chain variable domain comprises the amino acid sequence of SEQ ID NO: 376 and the heavy chain variable domain comprises the amino acid sequence of SEQ ID NO:551; (jj) the light chain variable domain comprises the amino acid sequence of SEQ ID NO: 387 and the heavy chain variable domain comprises the amino acid sequence of SEQ ID NO:561; (kk) the light chain variable domain comprises the amino acid sequence of SEQ ID NO: 398 and the heavy chain variable domain comprises the amino acid sequence of SEQ ID NO:571; (11) the light chain variable domain comprises the amino acid sequence of SEQ ID NO: 724 and the heavy chain variable domain comprises the amino acid sequence of SEQ ID NO:731; (mm) the light chain variable domain comprises the amino acid sequence of SEQ ID NO: 809 and the heavy chain variable domain comprises the amino acid sequence of SEQ ID NO:731; (nn) the light chain variable domain comprises the amino acid sequence of SEQ ID NO: 725 and the heavy chain variable domain comprises the amino acid sequence of SEQ ID NO:732; (oo) the light chain variable domain comprises the amino acid sequence of SEQ ID NO: 726 and the heavy chain variable domain comprises the amino acid sequence of SEQ ID NO:731; (pp) the light chain variable domain comprises the amino acid sequence of SEQ ID NO: 726 and the heavy chain variable domain comprises the amino acid sequence of SEQ ID NO:817; (qq) the light chain variable domain comprises the amino acid sequence of SEQ ID NO: 727 and the heavy chain variable domain comprises the amino acid sequence of SEQ ID NO:731; (rr) the light chain variable domain comprises the amino acid sequence of SEQ ID NO: 728 and the heavy chain variable domain comprises the amino acid sequence of SEQ ID NO:733; (ss) the light chain variable domain comprises the amino acid sequence of SEQ ID NO:810 and the heavy chain variable domain comprises the amino acid sequence of SEQ ID NO:818; (tt) the light chain variable domain comprises the amino acid sequence of SEQ ID NO:811 and the heavy chain variable domain comprises the amino acid sequence of SEQ ID NO:733; (uu) the light chain variable domain comprises the amino acid sequence of SEQ ID NO:729 and the heavy chain variable domain comprises the amino acid sequence of SEQ ID NO:731; (vv) the light chain variable domain comprises the amino acid sequence of SEQ ID NO:812 and the heavy chain variable domain comprises the amino acid sequence of SEQ ID NO:819; (ww) the light chain variable domain comprises the amino acid sequence of SEQ ID NO:729 and the heavy chain variable domain comprises the amino acid sequence of SEQ ID NO:820; (xx) the light chain variable domain comprises the amino acid sequence of SEQ ID NO: 730 and the heavy chain variable domain comprises the amino acid sequence of SEQ ID NO:731; (yy) the light chain variable domain comprises the amino acid sequence of SEQ ID NO:813 and the heavy chain variable domain comprises the amino acid sequence of SEQ ID NO:731; (zz) the light chain variable domain comprises the amino acid sequence of SEQ ID NO:814 and the heavy chain variable domain comprises the amino acid sequence of SEQ ID NO:822; (aaa) the light chain variable domain comprises the amino acid sequence of SEQ ID NO:815 and the heavy chain variable domain comprises the amino acid sequence of SEQ ID NO:824; or (bbb) the light chain variable domain comprises the amino acid sequence of SEQ ID NO:816 and the heavy chain variable domain comprises the amino acid sequence of SEQ ID NO:825.

Other aspects of the present disclosure relate to an isolated (e.g., monoclonal) antibody that binds to a TREM2 protein, wherein the antibody comprises a light chain variable domain of an antibody selected from the group consisting of: 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; and/or a heavy chain variable domain of an antibody selected from the group consisting of: 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2.

Other aspects of the present disclosure relate to an isolated (e.g., monoclonal) antibody that binds to a TREM2 protein, wherein the antibody binds essentially the same TREM2 epitope as an antibody selected from the group consisting of: 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2.

Other aspects of the present disclosure relate to an isolated (e.g., monoclonal) antibody that binds to a TREM2 protein, wherein the antibody comprises a light chain variable domain and a heavy chain variable domain, wherein the light chain variable domain comprises: (a) an HVR-L1 comprising an amino acid sequence selected from the group consisting of SEQ ID NOs: 9-23, 581, 690-694, 734-738, and 826-828, or an amino acid sequence with at least about 90% homology to an amino acid sequence selected from the group consisting of SEQ ID NOs: 9-23, 581, 690-694, 734-738, and 826-828; (b) an HVR-L2 comprising an amino acid sequence selected from the group consisting of SEQ ID NOs: 24-33, 695-697, and 739-743, or an amino acid sequence with at least about 90% homology to an amino acid sequence selected from the group consisting of SEQ ID NOs: 24-33, 695-697, and 739-743; and (c) an HVR-L3 comprising an amino acid sequence selected from the group consisting of SEQ ID NOs: 34-47, 582, 583, 698-702, and 744-746, or an amino acid sequence with at least about 90% homology to an amino acid sequence selected from the group consisting of SEQ ID NOs: 34-47, 582, 583, 698-702, and 744-746; and wherein the heavy chain variable domain comprises: (a) an HVR-H1 comprising an amino acid sequence selected from the group consisting of SEQ ID NOs: 48-65, 584, 703-705, 747-754, and 829-835, or an amino acid sequence with at least about 90% homology to an amino acid sequence selected from the group consisting of SEQ ID NOs: 48-65, 584, 703-705, 747-754, and 829-835; (b) an HVR-H2 comprising an amino acid sequence selected from the group consisting of SEQ ID NOs: 66-84, 585-587, 706-708, 755-762, 836-842, and 888, or an amino acid sequence with at least about 90% homology to an amino acid sequence selected from the group consisting of SEQ ID NOs: 66-84, 585-587, 706-708, 755-762, 836-842, and 888; and (c) an HVR-H3 comprising an amino acid sequence selected from the group consisting of SEQ ID NOs: 85-102, 588, 589, 709, 710, and 763-770, or an amino acid sequence with at least about 90% homology to an amino acid sequence selected from the group consisting of SEQ ID NOs: 85-102, 588, 589, 709, 710, and 763-770. Other aspects of the present disclosure relate to an isolated (e.g., monoclonal) antibody that binds to a TREM2 protein, wherein the anti-TREM2 antibody comprises a light chain variable domain and a heavy chain variable domain, wherein the light chain variable domain comprises HVR-L1, HVR-L2, HVR-L3, the heavy chain variable domain comprises HVR-H1, HVR-H2, and HVR-H3, and wherein the HVR-H3 comprises an amino acid sequence selected from the group consisting of SEQ ID NOs: 85-102, 588, 589, 709, 710, and 763-770, or an amino acid sequence with at least about 90% homology to an amino acid sequence selected from the group consisting of SEQ ID NOs: 85-102, 588, 589, 709, 710, and 763-770.

In some embodiments that may be combined with any of the preceding embodiments, the antibody competes with one or more TREM2 ligands for binding to the TREM2 protein. In some embodiments that may be combined with any of the preceding embodiments, the antibody induces one or more TRME2 activities and enhances one or more TREM2 activities induced by binding of one or more TREM2 ligands to the TREM2 protein. In some embodiments that may be combined with any of the preceding embodiments, the antibody induces one or more TRME2 activities without blocking binding of the one or more TREM2 ligands to the TREM2 protein. In some embodiments that may be combined with any of the preceding embodiments, the antibody induces one or more TRME2 activities without blocking binding of one or more TREM2 ligands to the TREM2 protein. In some embodiments that may be combined with any of the preceding embodiments, the antibody enhances the one or more TREM2 activities. In some embodiments that may be combined with any of the preceding embodiments, the antibody does not compete with the one or more TREM2 ligands for binding of to the TREM2 protein. In some embodiments that may be combined with any of the preceding embodiments, the antibody enhances binding of the one or more TREM2 ligands to the TREM2 protein. In some embodiments that may be combined with any of the preceding embodiments, the antibody enhances one or more TREM2 activities induced by binding of one or more TREM2 ligands to the TREM2 protein, as compared to the one or more TREM2 activities induced by binding of the one or more TREM2 ligands to the TREM2 protein in the absence of the isolated antibody. In some embodiments that may be combined with any of the preceding embodiments, the antibody synergizes with the one or more TREM2 ligands to enhance the one or more TREM2 activities. In some embodiments that may be combined with any of the preceding embodiments, the antibody enhances the one or more TREM2 activities in the absence of cell surface clustering of TREM2. In some embodiments that may be combined with any of the preceding embodiments, the antibody enhances the one or more TREM2 activities by inducing or retaining cell surface clustering of TREM2. In some embodiments that may be combined with any of the preceding embodiments, the antibody is clustered by an Fc-gamma receptor expressed on one or more immune cells. In some embodiments that may be combined with any of the preceding embodiments, the one or more immune cells are B cells or microglial cells. In some embodiments that may be combined with any of the preceding embodiments, the antibody increases levels of soluble TREM2, increases half-life of soluble TREM2, or both. In some embodiments that may be combined with any of the preceding embodiments, the levels of soluble TREM2 are selected from the group consisting of serum levels of TREM2, cerebral spinal fluid (CSF) levels of TREM2, tissue levels of TREM2, and any combination thereof. In some embodiments that may be combined with any of the preceding embodiments, the antibody decreases levels of TREM2 in one or more cells. In some embodiments that may be combined with any of the preceding embodiments, the antibody decreases cell surface levels of TREM2, decreases intracellular levels of TREM2, decreases total levels of TREM2, or any combination thereof. In some embodiments that may be combined with any of the preceding embodiments, the antibody induces TREM2 degradation, TREM2 cleavage, TREM2 internalization, TREM2 shedding, downregulation of TREM2 expression, or any combination thereof. In some embodiments that may be combined with any of the preceding embodiments, the levels of TREM2 in one or more cells are measured in primary cells selected from the group consisting of dendritic cells, bone marrow-derived dendritic cells, monocytes, microglia, macrophages, neutrophils, NK cells, osteoclasts, Langerhans cells of skin, and Kupffer cells, or on cell lines, and wherein the cellular levels of TREM2 are measured utilizing an in vitro cell assay. In some embodiments that may be combined with any of the preceding embodiments, the TREM2 protein is a mammalian protein or a human protein. In some embodiments that may be combined with any of the preceding embodiments, the TREM2 protein is a wild-type protein. In some embodiments that may be combined with any of the preceding embodiments, the TREM2 protein is a naturally occurring variant. In some embodiments that may be combined with any of the preceding embodiments, the TREM2 protein is expressed on human dendritic cells, human macrophages, human monocytes, human osteoclasts, human Langerhans cells of skin, human Kupffer cells, human microglia, or any combination thereof. In some embodiments that may be combined with any of the preceding embodiments, the one or more TREM2 activities are selected from the group consisting of: (a) TREM2 binding to DAP12; (b) TREM2 phosphorylation; (c) DAP12 phosphorylation; (d) activation of one or more tyrosine kinases, optionally wherein the one or more tyrosine kinases comprise a Syk kinase, ZAP70 kinase, or both; (e) activation of phosphatidylinositol 3-kinase (PI3K); (f) activation of protein kinase B (Akt); (g) recruitment of phospholipase C-gamma (PLC-gamma) to a cellular plasma membrane, activation of PLC-gamma, or both; (h) recruitment of TEC-family kinase dVav to a cellular plasma membrane; (i) activation of nuclear factor-rB (NF-rB); (j) inhibition of MAPK signaling; (k) phosphorylation of linker for activation of T cells (LAT), linker for activation of B cells (LAB), or both; (l) activation of IL-2-induced tyrosine kinase (Itk); (m) modulation of one or more pro-inflammatory mediators selected from the group consisting of IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (n) modulation of one or more anti-inflammatory mediators selected from the group consisting of IL-4, IL-10, TGF-β, IL-13, IL-35, IL-16, IFN-α, IL-1Rα, VEGF, G-CSF, YM, AXL, FLT1, and soluble receptors for TNF or IL-6, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (o) modulation of one or more genes whose expression is increased upon induction of inflammation, optionally wherein the one or more genes are selected from the group consisting of Fabp3, Fabp5, and LDR; (p) phosphorylation of extracellular signal-regulated kinase (ERK); (q) modulated expression of C-C chemokine receptor 7 (CCR7) in one or more cells selected from the group consisting of macrophages, M1 macrophages, activated M1 macrophages, M2 macrophages, dendritic cells, monocytes, osteoclasts, Langerhans cells of skin, Kupffer cells, microglia, M1 microglia, activated M1 microglia, and M2 microglia, and any combination thereof; (r) induction of microglial cell chemotaxis toward CCL19 and CCL21 expressing cells; (s) normalization of disrupted TREM2/DAP12-dependent gene expression; (t) recruitment of Syk, ZAP70, or both to a DAP12/TREM2 complex; (u) increasing activity of one or more TREM2-dependent genes, optionally wherein the one or more TREM2-dependent genes comprise nuclear factor of activated T-cells (NFAT) transcription factors; (v) increased maturation of dendritic cells, monocytes, microglia, M1 microglia, activated M1 microglia, and M2 microglia, macrophages, M1 macrophages, activated M1 macrophages, M2 macrophages, or any combination thereof; (w) increased ability of dendritic cells, monocytes, microglia, M1 microglia, activated M1 microglia, and M2 microglia, macrophages, M1 macrophages, activated M1 macrophages, M2 macrophages, or any combination thereof to prime or modulate the function of T cells, optionally wherein the T cells are one or more cells selected from the group consisting of CD8+ T cells, CD4+ T cells, regulatory T cells, and any combination thereof; (x) enhanced ability, normalized ability, or both of bone marrow-derived dendritic cells to prime or modulate function of antigen-specific T cells, optionally wherein the antigen-specific T cells are one or more cells selected from the group consisting of CD8+ T cells, CD4+T cells, regulatory T cells, and any combination thereof; (y) induction of osteoclast production, increased rate of osteoclastogenesis, or both; (z) increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; (aa) increasing the function of dendritic cells, macrophages, M1 macrophages, activated M1 macrophages, M2 macrophages, microglia, M1 microglia, activated M1 microglia, and M2 microglia, or any combination thereof; (bb) increasing phagocytosis by dendritic cells, macrophages, M1 macrophages, activated M1 macrophages, M2 macrophages, monocytes, microglia, M1 microglia, activated M1 microglia, and M2 microglia, or any combination thereof; (cc) induction of one or more types of clearance selected from the group consisting of apoptotic neuron clearance, nerve tissue debris clearance, non-nerve tissue debris clearance, bacteria or other foreign body clearance, disease-causing agent clearance, tumor cell clearance, or any combination thereof, optionally wherein the disease-causing agent is selected from the group consisting of amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products including DiPeptide Repeats, (DPRs peptides) composed of glycine-alanine (GA), glycine-proline (GP), glycine-arginine (GR), proline-alanine (PA), or proline-arginine (PR), antisense GGCCCC (G2C4) repeat-expansion RNA; (dd) induction of phagocytosis of one or more of apoptotic neurons, nerve tissue debris, non-nerve tissue debris, bacteria, other foreign bodies, disease-causing agents, tumor cells, or any combination thereof, optionally wherein the disease-causing agent is selected from the group consisting of amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products including DiPeptide Repeats, (DPRs peptides) composed of glycine-alanine (GA), glycine-proline (GP), glycine-arginine (GR), proline-alanine (PA), or proline-arginine (PR), antisense GGCCCC (G2C4) repeat-expansion RNA; (ee) increased expression of one or more stimulatory molecules selected from the group consisting of CD83, CD86 MHC class II, CD40, and any combination thereof, optionally wherein the CD40 is expressed on dendritic cells, monocytes, macrophages, or any combination thereof, and optionally wherein the dendritic cells comprise bone marrow-derived dendritic cells; (ff) modulating secretion of one or more pro-inflammatory mediators selected from the group consisting of IFN-β, IL-1α, IL-1β, CD86, TNF-α, IL-6, IL-8, CRP, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, and optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (gg) modulating secretion of one or more anti-inflammatory mediators selected from the group consisting of IL-4, IL-10 TGF-β, IL-13, IL-35 IL-16, IFN-alpha, IL-1Ra, VEGF, G-CSF, YM, AXL, FLT1, and soluble receptors for TNF or IL-6, and optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (hh) modulating expression of one or more proteins selected from the group consisting of C1qa, C1qB, C1qC, C1s, C1R, C4, C2, C3, ITGB2, HMOX1, LAT2. CASP1, CSTA, VSIG4, MS4A4A, C3AR1, GPX1, TyroBP, ALOX5AP, ITGAM, SLC7A7, CD4, ITGAX, PYCARD, and VEGF; (ii) increasing memory; and (jj) reducing cognitive deficit. In some embodiments that may be combined with any of the preceding embodiments, the one or more TREM2 activities are selected from the group consisting of: (a) TREM2 binding to DAP12; (b) DAP12 phosphorylation; (c) activation of Syk kinase; (d) modulation of one or more pro-inflammatory mediators selected from the group consisting of IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (e) recruitment of Syk to a DAP12/TREM2 complex; (f) increasing activity of one or more TREM2-dependent genes, optionally wherein the one or more TREM2-dependent genes comprise nuclear factor of activated T-cells (NFAT) transcription factors; (g) increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; (h) modulated expression of one or more stimulatory molecules selected from the group consisting of CD83, CD86 MHC class II, CD40, and any combination thereof, optionally wherein the CD40 is expressed on dendritic cells, monocytes, macrophages, or any combination thereof, and optionally wherein the dendritic cells comprise bone marrow-derived dendritic cells; (i) increasing memory; and (j) reducing cognitive deficit. In some embodiments that may be combined with any of the preceding embodiments, the antibody is of the IgG class the IgM class, or the IgA class. In some embodiments that may be combined with any of the preceding embodiments, the antibody is of the IgG class and has an IgG1, IgG2, IgG3, or IgG4 isotype. In some embodiments that may be combined with any of the preceding embodiments, the antibody has an IgG2 isotype. In some embodiments that may be combined with any of the preceding embodiments, the antibody comprises a human IgG2 constant region. In some embodiments that may be combined with any of the preceding embodiments, the human IgG2 constant region comprises an Fc region. In some embodiments that may be combined with any of the preceding embodiments, the antibody enhances the one or more TREM2 activities independently of binding to an Fc receptor. In some embodiments that may be combined with any of the preceding embodiments, the antibody binds an inhibitory Fc receptor. In some embodiments that may be combined with any of the preceding embodiments, the inhibitory Fc receptor is inhibitory Fc-gamma receptor IIB (FcγIIB). In some embodiments that may be combined with any of the preceding embodiments: (a) the isolated antibody has a human or mouse IgG1 isotype and comprises one or more amino acid substitutions in the Fc region at a residue position selected from the group consisting of: N297A, D265A, D270A, L234A, L235A, G237A, C226S, C229S, E233P, L234V, L234F, L235E, P331S, S267E, L328F, A330L, M252Y, S254T, T256E, L328E, P238D, S267E, L328F, E233D, G237D, H268D, P271G, A330R, and any combination thereof, wherein the numbering of the residues is according to EU numbering, or comprises an amino acid deletion in the Fc region at a position corresponding to glycine 236; (b) the isolated antibody has an IgG1 isotype and comprises an IgG2 isotype heavy chain constant domain 1(CH1) and hinge region, optionally wherein the IgG2 isotype CH1 and hinge region comprises the amino acid sequence of ASTKGPSVFP LAPCSRSTSE STAALGCLVK DYFPEPVTVS WNSGALTSGVHTFPAVLQSS GLYSLSSVVT VPSSNFGTQT YTCNVDHKPS NTKVDKTVERKCCVECPPCP (SEQ ID NO: 886), and optionally wherein the antibody Fc region comprises a S267E amino acid substitution, a L328F amino acid substitution, or both, and/or a N297A or N297Q amino acid substitution, wherein the numbering of the residues is according to EU numbering; (c) the isolated antibody has an IgG2 isotype and comprises one or more amino acid substitutions in the Fc region at a residue position selected from the group consisting of: P238S, V234A, G237A, H268A, H268Q, V309L, A330S, P331S, C214S, C232S, C233S, S267E, L328F, M252Y, S254T, T256E, H268E, N297A, N297Q, A330L, and any combination thereof, wherein the numbering of the residues is according to EU numbering; (d) the isolated antibody has a human or mouse IgG4 isotype and comprises one or more amino acid substitutions in the Fc region at a residue position selected from the group consisting of: L235A, G237A, S228P, L236E, S267E, E318A, L328F, M252Y, S254T, T256E, E233P, F234V, L234A/F234A, S228P, S241P, L248E, T394D, N297A, N297Q, L235E, and any combination thereof, wherein the numbering of the residues is according to EU numbering; or (e) the isolated antibody has a hybrid IgG2/4 isotype, and optionally wherein the antibody comprises an amino acid sequence comprising amino acids 118 to 260 of human IgG2 and amino acids 261 to 447 of human IgG4, wherein the numbering of the residues is according to EU or, Kabat numbering. In some embodiments that may be combined with any of the preceding embodiments, the antibody is an inert antibody that binds to a TREM2 protein. In some embodiments that may be combined with any of the preceding embodiments, the antibody is an antagonist antibody that binds to a TREM2 protein. In some embodiments that may be combined with any of the preceding embodiments, the TREM2 protein is a mammalian protein or a human protein. In some embodiments that may be combined with any of the preceding embodiments, the TREM2 protein is a wild-type protein. In some embodiments that may be combined with any of the preceding embodiments, the TREM2 protein is a naturally occurring variant. In some embodiments that may be combined with any of the preceding embodiments, the TREM2 protein is a disease variant. In some embodiments that may be combined with any of the preceding embodiments, the antibody inhibits one or more TREM2 activities. In some embodiments that may be combined with any of the preceding embodiments, the one or more TREM2 activities are selected from the group consisting of: (a) TREM2 binding to DAP12; (b) TREM2 phosphorylation; (c) DAP12 phosphorylation; (d) activation of one or more tyrosine kinases, optionally wherein the one or more tyrosine kinases comprise a Syk kinase, ZAP70 kinase, or both; (e) activation of phosphatidylinositol 3-kinase (PI3K); (f) activation of protein kinase B (Akt); (g) recruitment of phospholipase C-gamma (PLC-gamma) to a cellular plasma membrane, activation of PLC-gamma, or both; (h) recruitment of TEC-family kinase dVav to a cellular plasma membrane; (i) activation of nuclear factor-rB (NF-rB); (j) inhibition of MAPK signaling; (k) phosphorylation of linker for activation of T cells (LAT), linker for activation of B cells (LAB), or both; (1) activation of IL-2-induced tyrosine kinase (Itk); (m) modulation of one or more pro-inflammatory mediators selected from the group consisting of IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (n) modulation of one or more anti-inflammatory mediators selected from the group consisting of IL-4, IL-10 TGF-β, IL-13, IL-35 IL-16, IFN-alpha, IL-1Ra, VEGF, G-CSF, YM, AXL, FLT1 and soluble receptors for TNF or IL-6, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (o) modulation of one or more genes whose expression is increased upon induction of inflammation, optionally wherein the one or more genes are selected from the group consisting of Fabp3, Fabp5, and LDR; (p) phosphorylation of extracellular signal-regulated kinase (ERK); (q) increased expression of C-C chemokine receptor 7 (CCR7) in one or more cells selected from the group consisting of macrophages, M1 macrophages, activated M1 macrophages, M2 macrophages, dendritic cells, monocytes, osteoclasts, Langerhans cells of skin, Kupffer cells, microglia, M1 microglia, activated M1 microglia, and M2 microglia, and any combination thereof; (r) induction of microglial cell chemotaxis toward CCL19 and CCL21 expressing cells; (s) normalization of disrupted TREM2/DAP12-dependent gene expression; (t) recruitment of Syk, ZAP70, or both to a DAP12/TREM2 complex; (u) increasing activity of one or more TREM2-dependent genes, optionally wherein the one or more TREM2-dependent genes comprise nuclear factor of activated T-cells (NFAT) transcription factors; (v) promoting proliferation, maturation, migration, differentiation, or functionality of one or more cells selected from the group consisting of immunosuppressor dendritic cells, immunosuppressor macrophages, immunosuppressor neutrophils, immunosuppressor NK cells, myeloid derived suppressor cells, tumor-associated macrophages, tumor-associated suppressor neutrophils, tumor-associated suppressor NK cells, regulatory T cells, and any combination thereof; (w) enhancing infiltration into tumors of one or more cells selected from the group consisting of immunosuppressor dendritic cells, immunosuppressor macrophages, immunosuppressor neutrophils, immunosuppressor NK cells, myeloid-derived suppressor cells, tumor-associated macrophages, tumor-associated suppressor neutrophils, tumor-associated suppressor NK cells, regulatory T cells, and any combination thereof; (x) increasing number of tumor-promoting myeloid immunosuppressor or tumor-promoting granulocytic immunosuppressor cells in a tumor, peripheral blood, lymphoid organ, or any combination thereof; (y) enhancing tumor-promoting activity of myeloid-derived suppressor cells (MDSC); (z) increasing expression of tumor-promoting cytokines in a tumor or in peripheral blood, optionally wherein the tumor-promoting cytokines are selected from the group consisting of TGF-beta, IL-10, and any combination thereof; (aa) increasing tumor infiltration of tumor-promoting FoxP3+ regulatory T lymphocytes; (bb) decreasing activation of tumor-specific T lymphocytes with tumor-killing potential; (cc) decreasing infiltration of one of more cells selected from the group consisting of tumor-specific T lymphocytes with tumor killing potential, tumor-specific NK cells with tumor killing potential, tumor-specific B lymphocytes with potential to enhance immune response, and any combination thereof; (dd) increasing tumor volume; (ee) increasing tumor growth rate; (ff) increasing metastasis; (gg) increasing rate of tumor recurrence; (hh) decreasing efficacy of one or more immunotherapies that modulate anti-tumor T cell responses, optionally wherein the one or more immunotherapies are selected from the group consisting of PD1/PDL1 blockade, CTLA-4 blockade, and cancer vaccines; (ii) inhibition of PLCγ/PKC/calcium mobilization; and (jj) inhibition of PI3K/Akt, Ras/MAPK signaling. In some embodiments that may be combined with any of the preceding embodiments, the one or more TREM2 activities are selected from the group consisting of: (a) TREM2 binding to DAP12; (b) DAP12 phosphorylation; (c) activation of Syk kinase; (d) recruitment of Syk to a DAP12/TREM2 complex; (e) increasing activity of one or more TREM2-dependent genes, optionally wherein the one or more TREM2-dependent genes comprise nuclear factor of activated T-cells (NFAT) transcription factors; (f) increasing tumor volume; and (g) increasing tumor growth rate. In some embodiments that may be combined with any of the preceding embodiments, the antibody inhibits interaction between TREM2 and one or more TREM2 ligands, inhibits TREM2 signal transduction, or both. In some embodiments that may be combined with any of the preceding embodiments, the antibody is incapable of binding an Fc-gamma receptor (FcγR). In some embodiments that may be combined with any of the preceding embodiments, the antibody has an IgG1, IgG2, IgG3, or IgG4 isotype. In some embodiments that may be combined with any of the preceding embodiments: (a) the antibody has a human or mouse IgG1 isotype and comprises one or more amino acid substitutions in the Fc region at a residue position selected from the group consisting of: N297A, N297Q, D270A, D265A, L234A, L235A, C226S, C229S, P238S, E233P, L234V, P238A, A327Q, A327G, P329A, K322A, L234F, L235E, P331S, T394D, A330L, M252Y, S254T, T256E, L328E, P238D, S267E, L328F, E233D, G237D, H268D, P271G, A330R, and any combination thereof, wherein the numbering of the residues is according to EU numbering, or comprises an amino acid deletion in the Fc region at a position corresponding to glycine 236; (b) the antibody has an IgG2 isotype and comprises one or more amino acid substitutions in the Fc region at a residue position selected from the group consisting of: P238S, V234A, G237A, H268A, H268Q, H268E, V309L, N297A, N297Q, A330S, P331S, C232S, C233S, M252Y, S254T, T256E, and any combination thereof, wherein the numbering of the residues is according to EU numbering; or (c) the antibody has an IgG4 isotype and comprises one or more amino acid substitutions in the Fc region at a residue position selected from the group consisting of: E233P, F234V, L234A/F234A, L235A, G237A, E318A, S228P, L236E, S241P, L248E, T394D, M252Y, S254T, T256E, N297A, N297Q, and any combination thereof, wherein the numbering of the residues is according to EU numbering. In some embodiments that may be combined with any of the preceding embodiments: (a) the Fc region further comprises one or more additional amino acid substitutions at a position selected from the group consisting of A330L, L234F; L235E, P331S, and any combination thereof, wherein the numbering of the residues is according to EU numbering; (b) the Fc region further comprises one or more additional amino acid substitutions at a position selected from the group consisting of M252Y, S254T, T256E, and any combination thereof, wherein the numbering of the residues is according to EU numbering; or (c) the Fc region further comprises a S228P amino acid substitution according to EU numbering. In some embodiments that may be combined with any of the preceding embodiments, the antibody is an antibody fragment that binds to one or more human proteins selected from the group consisting of human TREM2, a naturally occurring variant of human TREM2, and a disease variant of human TREM2, and optionally wherein the antibody fragment is cross-linked to a second antibody fragment that binds to one or more human proteins selected from the group consisting of human TREM2, a naturally occurring variant of human TREM2, and a disease variant of human TREM2. In some embodiments that may be combined with any of the preceding embodiments, the fragment is an Fab, Fab′, Fab′-SH, F(ab′)2, Fv or scFv fragment. In some embodiments that may be combined with any of the preceding embodiments, the one or more TREM2 ligands are selected from the group consisting of E. coli cells, apoptotic cells, nucleic acids, anionic lipids, anionic lipids, APOE, APOE2, APOE3, APOE4, anionic APOE, anionic APOE2, anionic APOE3, anionic APOE4, lipidated APOE, lipidated APOE2, lipidated APOE3, lipidated APOE4, zwitterionic lipids, negatively charged phospholipids, phosphatidylserine, sulfatides, phosphatidylcholin, sphingomyelin, membrane phospholipids, lipidated proteins, proteolipids, lipidated peptides, lipidated amyloid beta peptide, and any combination thereof. In some embodiments that may be combined with any of the preceding embodiments, the antibody is a murine antibody. In some embodiments that may be combined with any of the preceding embodiments, the antibody is a humanized antibody, a bispecific antibody, a multivalent antibody, a conjugated antibody, or a chimeric antibody. In some embodiments that may be combined with any of the preceding embodiments, the antibody is a monoclonal antibody. In some embodiments that may be combined with any of the preceding embodiments, the antibody is a bispecific antibody recognizing a first antigen and a second antigen. In some embodiments that may be combined with any of the preceding embodiments, the first antigen is human TREM2 or a naturally occurring variant thereof, and the second antigen is: (a) an antigen facilitating transport across the blood-brain-barrier; (b) an antigen facilitating transport across the blood-brain-barrier selected from the group consisting of transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopeptide, and ANG1005; (c) a disease-causing agent selected from the group consisting of disease-causing peptides or proteins or, disease-causing nucleic acids, wherein the disease-causing nucleic acids are antisense GGCCCC (G2C4) repeat-expansion RNA, the disease-causing proteins are selected from the group consisting of amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9R AN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; (d) ligands and/or proteins expressed on immune cells, wherein the ligands and/or proteins selected from the group consisting of CD40, OX40, ICOS, CD28, CD137/4-1BB, CD27, GITR, PD-L1, CTLA-4, PD-L2, PD-1, B7-H3, B7-H4, HVEM, BTLA, KIR, GAL9, TIM3, A2AR, LAG-3, and phosphatidylserine; and (e) a protein, lipid, polysaccharide, or glycolipid expressed on one or more tumor cells. In some embodiments that may be combined with any of the preceding embodiments, the antibody is used in combination with one or more antibodies that specifically bind a disease-causing agent selected from the group consisting of disease-causing peptides, disease-causing proteins, amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and any combination thereof; or with one or more antibodies that bind an immunomodulatory protein selected from the group consisting of: CD40, OX40, ICOS, CD28, CD137/4-1BB, CD27, GITR, PD-L1, CTLA-4, PD-L2, PD-1, B7-H3, B7-H4, HVEM, BTLA, KIR, GAL9, TIM3, A2AR, LAG-3, TREM1, TREM2, CD33, Siglec-5, Siglec-9, Siglec-11, phosphatidylserine, disease-causing nucleic acids, antisense GGCCCC (G2C4) repeat-expansion RNA, and any combination thereof. In some embodiments that may be combined with any of the preceding embodiments, when administered to an individual increases memory, reduces cognitive deficit, or both. In some embodiments that may be combined with any of the preceding embodiments, the antibody binds specifically to both human TREM2 and mouse TREM2. In some embodiments that may be combined with any of the preceding embodiments, the antibody has dissociation constant (K_{D}) for human TREM2 and mouseTREM2 that ranges from about 12.8 nM to about 1.2 nM, or less than 1.2 nM. In some embodiments that may be combined with any of the preceding embodiments, the antibody has dissociation constant (K_{D}) for human TREM2 that ranges from about 12.8 nM to about 2.9 nM, or less than 2.9 nM. In some embodiments that may be combined with any of the preceding embodiments, the antibody has dissociation constant (K_{D}) for mouse TREM2 that ranges from about 10.4 nM to about 1.2 nM, or less than 1.2 nM. In some embodiments that may be combined with any of the preceding embodiments, the K_{D} is determined at a temperature of approximately 4° C. In some embodiments that may be combined with any of the preceding embodiments, the antibody does not inhibit the growth of innate immune cells. In some embodiments that may be combined with any of the preceding embodiments, the antibody binds to primary immune cells with a K_{D} of less than 1 nM. In some embodiments that may be combined with any of the preceding embodiments, the antibody accumulates in the brain, or the cerebrospinal fluid (CSF), or both to an extent that is 1% or more of the concentration of the antibody in the blood. In some embodiments that may be combined with any of the preceding embodiments, the antibody accumulates in the brain, or the cerebrospinal fluid (CSF), or both to an extent that is 2% or more of the concentration of the antibody in the blood. In some embodiments that may be combined with any of the preceding embodiments, the antibody accumulates in the brain, or the cerebrospinal fluid (CSF), or both to an extent that is 3% or more of the concentration of the antibody in the blood. In some embodiments that may be combined with any of the preceding embodiments, the antibody accumulates in the brain, or the cerebrospinal fluid (CSF), or both to an extent that is 4% or more of the concentration of the antibody in the blood.

Other aspects of the present disclosure relate to an isolated nucleic acid comprising a nucleic acid sequence encoding the antibody of any one of the preceding embodiments. Other aspects of the present disclosure relate to a vector comprising the nucleic acid of any of the preceding embodiments. Other aspects of the present disclosure relate to an isolated host cell comprising the vector of any of the preceding embodiments. Other aspects of the present disclosure relate to a method of producing an antibody that binds to TREM2, comprising culturing the host cell of any of the preceding embodiments so that the antibody is produced. In some embodiments, the method further comprising recovering the antibody produced by the cell. Other aspects of the present disclosure relate to an isolated (e.g., monoclonal) antibody that binds to TREM2 produced by the method of any of the preceding embodiments. Other aspects of the present disclosure relate to a pharmaceutical composition comprising the antibody of any of the preceding embodiments and a pharmaceutically acceptable carrier.

Other aspects of the present disclosure relate to a method of preventing, reducing risk, or treating an individual having a disease, disorder, or injury selected from the group consisting of dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an isolated (e.g., monoclonal) antibody that binds to a TREM2 protein. Other aspects of the present disclosure relate to an isolated (e.g., monoclonal) antibody that binds to a TREM2 protein for use in preventing, reducing risk, or treating an individual having a disease, disorder, or injury selected from the group consisting of dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza. Other aspects of the present disclosure relate to use of an isolated (e.g., monoclonal) antibody that binds to a TREM2 protein in the manufacture of a medicament for preventing, reducing risk, or treating an individual having a disease, disorder, or injury selected from the group consisting of dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza. Other aspects of the present disclosure relate to a method of preventing, reducing risk, or treating an individual having a disease, disorder, or injury selected from the group consisting of dementia, frontotemporal dementia, Alzheimer's disease, Nasu-Hakola disease, cognitive deficit, memory loss, spinal cord injury, traumatic brain injury, multiple sclerosis, chronic colitis, ulcerative colitis in and cancer, comprising administering to an individual in need thereof a therapeutically effective amount of an isolated (e.g., monoclonal) antibody that binds to a TREM2 protein. Other aspects of the present disclosure relate to an isolated (e.g., monoclonal) antibody that binds to a TREM2 protein for use in preventing, reducing risk, or treating an individual having a disease, disorder, or injury selected from the group consisting of dementia, frontotemporal dementia, Alzheimer's disease, Nasu-Hakola disease, cognitive deficit, memory loss, spinal cord injury, traumatic brain injury, multiple sclerosis, chronic colitis, ulcerative colitis in and cancer. Other aspects of the present disclosure relate to use of an isolated (e.g., monoclonal) antibody that binds to a TREM2 protein in the manufacture of a medicament for preventing, reducing risk, or treating an individual having a disease, disorder, or injury selected from the group consisting of dementia, frontotemporal dementia, Alzheimer's disease, Nasu-Hakola disease, cognitive deficit, memory loss, spinal cord injury, traumatic brain injury, multiple sclerosis, chronic colitis, ulcerative colitis in and cancer. In some embodiments, the isolated antibody is: (a) an agonist antibody; (b) an inert antibody; or (c) an antagonist antibody. In some embodiments, the isolated antibody is the antibody of any of the preceding embodiments. In some embodiments, the disease, disorder, or injury is Alzheimer's disease. In some embodiments, the isolated antibody that binds to a TREM2 protein increases expression of one or more inflammatory mediators, wherein the one or more inflammatory mediators are selected from the group consisting of IL-1β, TNF-α, YM-1, CD86, CCL2, CCL3, CCL5, CCR2, CXCL10, Gata3, Rorc, and any combination thereof. hi some embodiments, the isolated antibody that binds to a TREM2 protein decreases expression of one or more inflammatory mediators, wherein the one or more inflammatory mediators are selected from the group consisting of FLT1, OPN, CSF-1, CD11c, AXL, and any combination thereof. In some embodiments, the isolated antibody that binds to a TREM2 protein decreases levels of Abeta peptide in the individual. In some embodiments, the isolated antibody that binds to a TREM2 protein increases the number of CD11b⁺ microglial cells in the brain of the individual. In some embodiments, the isolated antibody that binds to a TREM2 protein increases memory of the individual. In some embodiments, the isolated antibody that binds to a TREM2 protein reduces cognitive deficit in the individual. In some embodiments, the isolated antibody that binds to a TREM2 protein increases motor coordination in the individual. In some embodiments, the method further comprises administering to the individual at least one antibody that specifically binds to an inhibitory checkpoint molecule, and/or another standard or investigational anti-cancer therapy. In some embodiments, the at least one antibody that specifically binds to an inhibitory checkpoint molecule is administered in combination with the isolated antibody. In some embodiments, the at least one antibody that specifically binds to an inhibitory checkpoint molecule is selected from the group consisting of an anti-PD-L1 antibody, an anti-CTLA-4 antibody, an anti-PD-L2 antibody, an anti-PD-1 antibody, an anti-B7-H3 antibody, an anti-B7-H4 antibody, and anti-HVEM antibody, an anti-B- and T-lymphocyte attenuator (BTLA) antibody, an anti-Killer inhibitory receptor (KIR) antibody, an anti-GALS antibody, an anti-TIM3 antibody, an anti-A2AR antibody, an anti-LAG-3 antibody, an anti-phosphatidylserine antibody, an anti-CD27 antibody, and any combination thereof. In some embodiments, the standard or investigational anti-cancer therapy is one or more therapies selected from the group consisting of radiotherapy, cytotoxic chemotherapy, targeted therapy, hormonal therapy, imatinib (Gleevec®), trastuzumab (Herceptin®), bevacizumab (Avastin®), Ofatumumab (Arzerra®), Rituximab (Rituxan®, MabThera®, Zytux®), cryotherapy, ablation, radiofrequency ablation, adoptive cell transfer (ACT), chimeric antigen receptor T cell transfer (CAR-T), vaccine therapy, and cytokine therapy. In some embodiments, the method further comprises administering to the individual at least one antibody that specifically binds to an inhibitory cytokine. In some embodiments, the at least one antibody that specifically binds to an inhibitory cytokine is administered in combination with the isolated antibody. In some embodiments, the at least one antibody that specifically binds to an inhibitory cytokine is selected from the group consisting of an anti-CCL2 antibody, an anti-CSF-1 antibody, an anti-IL-2 antibody, and any combination thereof. In some embodiments, the method further comprises administering to the individual at least one agonistic antibody that specifically binds to a stimulatory checkpoint protein. In some embodiments, the at least one agonistic antibody that specifically binds to a stimulatory checkpoint protein is administered in combination with the isolated antibody. In some embodiments, the at least one agonistic antibody that specifically binds to a stimulatory checkpoint protein is selected from the group consisting of an agonist anti-CD40 antibody, an agonist anti-OX40 antibody, an agonist anti-ICOS antibody, an agonist anti-CD28 antibody, an agonist anti-CD137/4-1BB antibody, an agonist anti-CD27 antibody, an agonist anti-glucocorticoid-induced TNFR-related protein GITR antibody, and any combination thereof. In some embodiments, the method further comprises administering to the individual at least one stimulatory cytokine. In some embodiments, the at least one stimulatory cytokine is administered in combination with the isolated antibody. In some embodiments, the at least one stimulatory cytokine is selected from the group consisting of TNF-α, IL-10, IL-6, IL-8, CRP, TGF-beta members of the chemokine protein families, IL20 family member, IL-33, LIF, OSM, CNTF, TGF-beta, IL-11, IL-12, IL-17, IL-8, IL-23, IFN-α, IFN-β, IL-2, IL-18, GM-CSF, G-CSF, and any combination thereof.

Other aspects of the present disclosure relate to a method of enhancing one or more TREM2 activities induced by binding of one or more TREM2 ligands to a TREM2 protein in an individual in need thereof, comprising administering to the individual a therapeutically effective amount of an isolated (e.g., monoclonal) antibody that binds to a TREM2 protein. Other aspects of the present disclosure relate to an isolated (e.g., monoclonal) antibody that binds to a TREM2 protein for use in enhancing one or more TREM2 activities induced by binding of one or more TREM2 ligands to a TREM2 protein in an individual in need thereof. Other aspects of the present disclosure relate to use of an isolated (e.g., monoclonal) antibody that binds to a TREM2 protein in the manufacture of a medicament for enhancing one or more TREM2 activities induced by binding of one or more TREM2 ligands to a TREM2 protein in an individual in need thereof. In some embodiments, the isolated antibody is the antibody of any of the preceding embodiments.

Other aspects of the present disclosure relate to a method of inducing one or more TREM2 activities in an individual in need thereof, comprising administering to the individual a therapeutically effective amount of an isolated (e.g., monoclonal) antibody that binds to a TREM2 protein. Other aspects of the present disclosure relate to an isolated (e.g., monoclonal) antibody that binds to a TREM2 protein for use in inducing one or more TREM2 activities in an individual in need thereof. Other aspects of the present disclosure relate to use of an isolated (e.g., monoclonal) antibody that binds to a TREM2 protein in the manufacture of a medicament for inducing one or more TREM2 activities in an individual in need thereof. In some embodiments, the isolated antibody is the antibody of any of the preceding embodiments.

Other aspects of the present disclosure relate to a method of inducing one or more TREM2 activities and enhancing one or more TREM2 activities induced by binding of one or more TREM2 ligands to a TREM2 protein in an individual in need thereof, comprising administering to the individual a therapeutically effective amount of an isolated (e.g., monoclonal) antibody that binds to a TREM2 protein. Other aspects of the present disclosure relate to an isolated (e.g., monoclonal) antibody that binds to a TREM2 protein for use in inducing one or more TREM2 activities and enhancing one or more TREM2 activities induced by binding of one or more TREM2 ligands to a TREM2 protein in an individual in need thereof. Other aspects of the present disclosure relate to use of an isolated (e.g., monoclonal) antibody that binds to a TREM2 protein in the manufacture of a medicament for inducing one or more TREM2 activities and enhancing one or more TREM2 activities induced by binding of one or more TREM2 ligands to a TREM2 protein in an individual in need thereof. In some embodiments, the isolated antibody is the antibody of any of the preceding embodiments.

Other aspects of the present disclosure relate to a method of decreasing cellular levels of TREM2 in one or more cells in an individual in need thereof, comprising administering to the individual a therapeutically effective amount of an isolated (e.g., monoclonal) antibody that binds to a TREM2 protein. Other aspects of the present disclosure relate to an isolated (e.g., monoclonal) antibody that binds to a TREM2 protein for use in decreasing cellular levels of TREM2 in one or more cells in an individual in need thereof. Other aspects of the present disclosure relate to use of an isolated (e.g., monoclonal) antibody that binds to a TREM2 protein in the manufacture of a medicament for decreasing cellular levels of TREM2 in one or more cells in an individual in need thereof. In some embodiments, the isolated antibody is the antibody of any of the preceding embodiments.

In some embodiments that may be combined with any of the preceding embodiments, the individual has a heterozygous variant of TREM2, wherein the variant comprises one or more substitutions selected from the group consisting of: i. a glutamic acid to stop codon substitution in the nucleic acid sequence encoding amino acid residue Glu14 of SEQ ID NO: 1; ii. a glutamine to stop codon substitution in the nucleic acid sequence encoding amino acid residue Gln33 of SEQ ID NO: 1; iii. a tryptophan to stop codon substitution in the nucleic acid sequence encoding amino acid residue Trp44 of SEQ ID NO: 1; iv. an arginine to histidine amino acid substitution at an amino acid corresponding to amino acid residue Arg47 of SEQ ID NO: 1; v. a tryptophan to stop codon substitution in the nucleic acid sequence encoding amino acid residue Trp78 of SEQ ID NO: 1; vi. a valine to glycine amino acid substitution at an amino acid corresponding to amino acid residue Va1126 of SEQ ID NO: 1; vii. an aspartic acid to glycine amino acid substitution at an amino acid corresponding to amino acid residue Asp134 of SEQ ID NO: 1; and viii. a lysine to asparagine amino acid substitution at an amino acid corresponding to amino acid residue Lys186 of SEQ ID NO: 1. In some embodiments that may be combined with any of the preceding embodiments, the individual has a heterozygous variant of TREM2, wherein the variant comprises a guanine nucleotide deletion at a nucleotide corresponding to nucleotide residue G313 of the nucleic acid sequence encoding SEQ ID NO: 1; a guanine nucleotide deletion at a nucleotide corresponding to nucleotide residue G267 of the nucleic acid sequence encoding SEQ ID NO: 1; or both. In some embodiments that may be combined with any of the preceding embodiments, the individual has a heterozygous variant of DAP12, wherein the variant comprises one or more variants selected from the group consisting of: i. a methionine to threonine substitution at an amino acid corresponding to amino acid residue Mal of SEQ ID NO: 2; ii. a glycine to arginine amino acid substitution at an amino acid corresponding to amino acid residue Gly49 of SEQ ID NO: 2; iii. a deletion within exons 1-4 of the nucleic acid sequence encoding SEQ ID NO: 2; iv. an insertion of 14 amino acid residues at exon 3 of the nucleic acid sequence encoding SEQ ID NO: 2; and v. a guanine nucleotide deletion at a nucleotide corresponding to nucleotide residue G141 of the nucleic acid sequence encoding SEQ ID NO: 2.

Other aspects of the present disclosure relate to a method of inducing or promoting innate immune cell survival or wound healing an individual in need thereof, comprising administering to the individual a therapeutically effective amount of an isolated agonist antibody that binds to a TREM2 protein. Other aspects of the present disclosure relate to an isolated agonist antibody that binds to a TREM2 protein for use in inducing or promoting innate immune cell survival or wound healing an individual in need thereof. Other aspects of the present disclosure relate to use of an isolated agonist antibody that binds to a TREM2 protein in the manufacture of a medicament for inducing or promoting innate immune cell survival or wound healing an individual in need thereof. In some embodiments, the isolated agonist antibody is the agonist antibody of any of the preceding embodiments.

Other aspects of the present disclosure relate to a method of increasing memory, reducing cognitive deficit, or both in an individual in need thereof, comprising administering to the individual a therapeutically effective amount of an isolated agonist antibody that binds to a TREM2 protein. Other aspects of the present disclosure relate to an isolated agonist antibody that binds to a TREM2 protein for use in increasing memory, reducing cognitive deficit, or both in an individual in need thereof. Other aspects of the present disclosure relate to use of an isolated agonist antibody that binds to a TREM2 protein in the manufacture of a medicament for increasing memory, reducing cognitive deficit, or both in an individual in need thereof. In some embodiments, the isolated agonist antibody is the agonist antibody of any of the preceding embodiments.

Other aspects of the present disclosure relate to a method of increasing motor coordination in an individual in need thereof, comprising administering to the individual a therapeutically effective amount of an isolated agonist antibody that binds to a TREM2 protein. Other aspects of the present disclosure relate to an isolated agonist antibody that binds to a TREM2 protein for use in increasing motor coordination in an individual in need thereof. Other aspects of the present disclosure relate to use of an isolated agonist antibody that binds to a TREM2 protein in the manufacture of a medicament for increasing motor coordination in an individual in need thereof. In some embodiments, the isolated agonist antibody is the agonist antibody of any of the preceding embodiments.

Other aspects of the present disclosure relate to a method of reducing Abeta peptide levels in an individual in need thereof, comprising administering to the individual a therapeutically effective amount of an isolated agonist antibody that binds to a TREM2 protein. Other aspects of the present disclosure relate to an isolated agonist antibody that binds to a TREM2 protein for use in reducing Abeta peptide levels in an individual in need thereof. Other aspects of the present disclosure relate to use of an isolated agonist antibody that binds to a TREM2 protein in the manufacture of a medicament for reducing Abeta peptide levels an individual in need thereof. In some embodiments, the isolated agonist antibody is the agonist antibody of any of the preceding embodiments.

Other aspects of the present disclosure relate to a method of increasing the number of CD11b⁺ microglial cells in an individual in need thereof, comprising administering to the individual a therapeutically effective amount of an isolated agonist antibody that binds to a TREM2 protein. Other aspects of the present disclosure relate to an isolated agonist antibody that binds to a TREM2 protein for use in increasing the number of CD11b⁺ microglial cells in an individual in need thereof. Other aspects of the present disclosure relate to use of an isolated agonist antibody that binds to a TREM2 protein in the manufacture of a medicament for increasing the number of CD11b⁺ microglial cells in an individual in need thereof. In some embodiments, the isolated agonist antibody is the agonist antibody of any of the preceding embodiments.

Other aspects of the present disclosure relate to a method of increasing levels of one or more of FLT1, OPNCSF1, CD11c, and AXL in an individual in need thereof, comprising administering to the individual a therapeutically effective amount of an isolated agonist antibody that binds to a TREM2 protein. Other aspects of the present disclosure relate to an isolated agonist antibody that binds to a TREM2 protein for use in increasing levels of one or more of FLT1, OPNCSF1, CD11c, and AXL in an individual in need thereof. Other aspects of the present disclosure relate to use of an isolated agonist antibody that binds to a TREM2 protein in the manufacture of a medicament for increasing levels of one or more of FLT1, OPNCSF1, CD11c, and AXL in an individual in need thereof. In some embodiments, the isolated agonist antibody is the agonist antibody of any of the preceding embodiments.

Other aspects of the present disclosure relate to a method of treating spinal cord injury in an individual in need thereof, comprising administering to the individual a therapeutically effective amount of an isolated agonist antibody that binds to a TREM2 protein. Other aspects of the present disclosure relate to an isolated agonist antibody that binds to a TREM2 protein for use in treating spinal cord injury in an individual in need thereof. Other aspects of the present disclosure relate to use of an isolated agonist antibody that binds to a TREM2 protein in the manufacture of a medicament for treating spinal cord injury in an individual in need thereof. In some embodiments, the isolated agonist antibody is the agonist antibody of any of the preceding embodiments.

Other aspects of the present disclosure relate to a method of treating s chronic colitis or ulcerative colitis in an individual in need thereof, comprising administering to the individual a therapeutically effective amount of an isolated agonist antibody that binds to a TREM2 protein. Other aspects of the present disclosure relate to an isolated agonist antibody that binds to a TREM2 protein for use in treating chronic colitis or ulcerative colitis in an individual in need thereof. Other aspects of the present disclosure relate to use of an isolated agonist antibody that binds to a TREM2 protein in the manufacture of a medicament for treating chronic colitis or ulcerative colitis in an individual in need thereof. In some embodiments, the isolated agonist antibody is the agonist antibody of any of the preceding embodiments.

In some embodiments that may be combined with any of the preceding embodiments, the antibody does not inhibit the growth of innate immune cells. In some embodiments that may be combined with any of the preceding embodiments, the antibody binds to primary immune cells with a K_{D} of less than 1 nM. In some embodiments that may be combined with any of the preceding embodiments, the K_{D} is determined at a temperature of approximately 4° C. In some embodiments that may be combined with any of the preceding embodiments, the antibody accumulates in the brain, or the cerebrospinal fluid (CSF), or both to an extent that is 1% or more of the concentration of the antibody in the blood. In some embodiments that may be combined with any of the preceding embodiments, the antibody accumulates in the brain, or the cerebrospinal fluid (CSF), or both to an extent that is 2% or more of the concentration of the antibody in the blood. In some embodiments that may be combined with any of the preceding embodiments, the antibody accumulates in the brain, or the cerebrospinal fluid (CSF), or both to an extent that is 3% or more of the concentration of the antibody in the blood. In some embodiments that may be combined with any of the preceding embodiments, the antibody accumulates in the brain, or the cerebrospinal fluid (CSF), or both to an extent that is 4% or more of the concentration of the antibody in the blood.

### BRIEF DESCRIPTION OF THE DRAWINGS

FIG. 1A shows an amino acid sequence alignment between the human TREM2 protein (SEQ ID NO: 1) and the human NCTR2 protein (SEQ ID NO: 851), depicting the homology between the two proteins. The consensus sequence is SEQ ID NO: 852.

FIG. 1B shows a structure-based sequence alignment between several TREM proteins and other members of the IgV family. The amino acid residue numbering is consistent with the mature sequence of the human TREM1 protein. The secondary structure elements of TREM1 are illustrated as arrows for the β strands and cylinders for a helices Amino acid residues involved in homo- and heterodimer formation are shown on black background. Cysteine residues that form disulfide bonds and that are conserved for the V-type Ig fold, are depicted in bold and marked with asterisks. Gaps are indicated by “−”. M−1 residues violating antibody-like dimer formation mode are marked with closed triangles as (e.g., Radaev et al., (2003) Structure. 11(12):1527-1535). TREM-1_human (SEQ ID NO: 853), TREM-2_human (SEQ ID NO: 854), TREM-1_mouse (SEQ ID NO: 855), TREM-2_mouse (SEQ ID NO: 856), TREM-3_mouse (SEQ ID NO: 857), NKp44 (SEQ ID NO: 858), aTCR_human (SEQ ID NO: 859), bTCR_human (SEQ ID NO: 860), gTCR_human (SEQ ID NO: 861), dTCR_human (SEQ ID NO: 862), Vd_human (SEQ ID NO: 863), hIGG1_mouse (SEQ ID NO: 864), 1IGG1_mouse (SEQ ID NO: 865), CD8_human (SEQ ID NO: 866), and CTLA4_human (SEQ ID NO: 867).

FIG. 2 shows an amino acid sequence alignment between the human TREM1 protein (SEQ ID NO: 868) and the human TREM2 protein (SEQ ID NO: 1), depicting the homology between the two proteins. The consensus sequence is SEQ ID NO: 869.

FIG. 3A shows FACS histograms demonstrating binding of TREM2 antibodies 7E5 and 2H8 to a mouse cell line (BWZ) expressing recombinant mouse TREM2. FIG. 3B shows antibodies 7E5 and 2H8 binding to wild-type (TREM2+/+) bone marrow derived mouse macrophages (BMMac) and TREM2-deficient (TREM2−/−) BMMac. Antibody mIgG1 represents a negative isotype control. Shaded histograms represent the TREM2 negative cells population. Black outlined histograms represent the TREM2 positive cell population. FIG. 3C shows a dose response curve demonstrating dose-dependent binding of the TREM2 antibody 7E5 to BWZ cells expressing recombinant mouse TREM2 but not to parental BWZ cells. Antibody mIgG1 represents the negative isotype control.

FIG. 4A shows FACS histograms demonstrating binding of TREM2 antibodies 10A9, 10C1, and 8F8 to a human cell line (293) expressing a recombinant human TREM2-DAP12 fusion protein. Shaded histograms represent a TREM2 negative cell population. Black outlined histograms represent a TREM2 positive cell population. FIG. 4B shows antibodies 10A9, 10C1, and 8F8 binding to primary human dendritic cells (hDCs). Shaded histograms show binding of the isotype antibody negative control. Black outlined histograms represent binding of the TREM2 antibodies. FIG. 4C shows a schematic for combining antibody light chain variable region (VL) sequences of humanized versions of anti-TREM2 antibody 9F5 (mAb T2-9F5.1). Additional variations are listed below each sequence. The figure includes sequences for versions of humanized antibody 9F5. In the figure, IGKV2-29*02 (SEQ ID NO: 870); Joining region (SEQ ID NO: 871); T2-9F5.1 (SEQ ID NO: 872); 2-29*02 (SEQ ID NO: 873); h9F5-L1 (SEQ ID NO: 874); h9F5-L2 (SEQ ID NO: 875). FIG. 4D shows a schematic for combining antibody heavy chain variable region (VH) sequences of humanized versions of anti-TREM2 antibody 9F5 (mAb T2-9F5.1). Additional variations are listed below each sequence. The figure includes sequences for versions of humanized antibody 9F5. In the figure, IGHV1-46*01 (SEQ ID NO: 876); Joining region (SEQ ID NO: 877); T2-9F5.1 (SEQ ID NO: 878); 1-46*01 (SEQ ID NO: 879); h9F5-H1 (SEQ ID NO: 880); h9F5-H2 (SEQ ID NO: 881); h9F5-H3 (SEQ ID NO: 882). FIG. 4E shows binding reactivity in percentage to wild-type TREM2 (% WT) of the anti-TREM2 antibody 9F5 (MAb), as well as the anti-TREM2 antibodies T21-9 (Fab), T22 (Fab), and T45-10 (Fab), to the indicated TREM2 mutants.

FIG. 5A shows Syk phosphorylation as determined by Western blot analysis in mouse bone marrow derived macrophages after incubation with TREM2 antibodies 2F6, 11H5, 2H8, 1H7, 3A7, 3B10, 10A9, 7F8, and 7E5. As a control cells were left untreated (NT) or incubated with mIgG1 isotype control do not induce Syk phosphorylation. FIG. 5B shows Syk phosphorylation as determined by western blot in WT, Fc receptor common gamma chain-deficient (FcgR−/−) and TREM2-deficient (TREM2−/−) bone marrow derived mouse macrophages after incubation with the TREM2 antibodies 7E5, 3A7, and 2F6.

FIG. 6A shows Syk phosphorylation as determined by Western blot in wild-type (WT) and TREM2-deficient (TREM2−/−) bone marrow derived mouse macrophages that were untreated (NT), or treated with TREM2 antibodies 7E5, 3A7, 8F8, and 2F6 in the presence of a P815 cell line that overexpresses Fc receptors FcR2b and FcR3. Antibody IgG1 is the isotype control. FIG. 6B shows Syk phosphorylation as determined by western blot in WT bone marrow derived mouse macrophages that were untreated (NT), or treated with TREM2 antibodies 7E5, 3A7, 8F8, and 2F6 in the presence of primary murine B cells that express endogenous Fc receptor FcR2b. Antibody IgG1 is the isotype control.

FIG. 7A shows DAP12 phosphorylation (pTyr) as determined by Western blot in mouse macrophages after incubation with the TREM2 antibodies 11A2, 11H5, 2F6, 3A7, 4G3, 12F9, 3B10, and 7A9, or left untreated (NT). Antibody mIgG1 is the isotype negative control. FIG. 7B shows DAP12 phosphorylation as determined by Western blot in wild-type (WT) and TREM2-deficient (TREM2−/−) mouse macrophages that were untreated (NT), or treated with TREM2 antibodies 7E5 and 2F6. FIG. 7C shows DAP12 phosphorylation as determined by Western blot immunoprecipitation in peritoneal cells from mice that were treated with control antibody MOPC.1 or TREM2 antibody 7E5 for 15 minutes. FIG. 7D shows fold-change over IP-TREM2 of MOPC1-treated mouse for the 15 minute treatment. FIG. 7E shows DAP12 phosphorylation as determined by Western blot immunoprecipitation in peritoneal cells from mice that were treated with control antibody MOPC.1 or TREM2 antibody 7E5 for 24 hours. FIG. 7F shows fold-change over IP-TREM2 of MOPC1-treated mouse for the 24 hour treatment.

FIG. 8A shows induction of mouse TREM2-dependent luciferase reporter in a cell-based assay. Cells were either untreated (NT) or treated with plate-bound, full-length anti-TREM2 antibodies 1H7, 2F6, 2H8, 3A7, 3B10, 7E5, 7F8, 8F8, and 11H5. Results are expressed as fold over background. The background level is depicted by the dotted line. FIG. 8B shows induction of human TREM2-dependent luciferase reporter in a cell-based assay. Cells were either untreated (NT) or treated with plate-bound, full-length anti-TREM2 antibodies 9F5, 9G1, 9G3, 10A9, 10C1, 11A8, 12D9, 12E2, 12F9, 12G6, 2C7, 2F5, 3C1, and 4D7. Results are expressed as fold over background. Antibody msIgG1 is the isotype negative control. Cells treated with PMA/Ionomycin (P+I) represent the positive control. FIG. 8C shows induction of mouse TREM2-dependent luciferase reporter gene expression by increasing concentrations of plate-bound phosphatidylserine (PS) or sphingomyelin (SM). Results are expressed as absolute luminescence values. FIG. 8D shows induction of human TREM2-dependent luciferase reporter, gene expression by increasing concentrations of plate-bound phosphatidylserine (PS) or sphingomyelin (SM). Results are expressed as absolute luminescence values. FIG. 8E shows induction of human TREM2-dependent luciferase reporter gene expression by increasing concentrations of Apolipoprotein E (APOE). Three different alleles of APOE (APOE2, APOE3 and APOE4) were tested. Results are expressed as absolute luminescence values. FIG. 8F shows binding of APOE2, APOE3 and APOE4 to a recombinant human TREM2 protein as detected by ELISA. Results are expressed as OD₄₅₀.

FIG. 9A shows induction of mouse TREM2-dependent luciferase reporter in a cell-based assay. Cells were either untreated (NT) or treated with soluble full-length anti-TREM2 antibodies 1H7, 2F6, 2H8, 3A7, 3B10, 7E5, 7F8, 8F8, and 11H5. Antibody mIgG1 is the isotype negative control. Cell treated with PMA/Ionomycin represent the positive control. Results are expressed as fold over background (represented by the dotted line). FIG. 9B shows induction of human TREM2-dependent luciferase reporter expression by full-length anti-TREM2 antibodies 9F5, 9G1, 9G3, 10A9, 10C1, 11A8, 12D9, 12E2, 12F9, 12G6, 2C7, 2F5, 3C1, and 4D7 in solution. Antibody mIgG1 is the isotype negative control. Cells treated with PMA/Ionomycin represent the positive control. Results are expressed as fold over background (represented by the dotted line). FIG. 9C shows a dose dependent induction of the TREM2 luciferase reporter in a cell-based assay. Cells were either untreated (NT) or treated with increasing concentrations of the full-length anti-TREM2 antibody 7E5 in solution. Results are expressed as absolute luminescence values. Data was analyzed with Prism6 software and fitted with log (agonist) vs. response four parameter variable slope. EC50=1.52 nM.

FIG. 10A shows induction of mouse TREM2-dependent luciferase reporter gene expression by the indicated amounts of full-length anti-TREM2 antibody 7E5 added in solution, and in conjunction with increasing concentrations of plate-bound phosphatidylserine (PS). FIG. 10B shows induction of mouse TREM2-dependent luciferase reporter gene expression by the indicated amounts of full-length IgG1 isotype control antibody added in solution, and in conjunction with increasing concentrations of plate-bound phosphatidylserine (PS). FIG. 10C shows induction of mouse TREM2-dependent luciferase reporter gene expression by the indicated amounts of full-length anti-TREM2 antibody 7E5 added in solution, and in conjunction with increasing concentrations of plate-bound sphingomyelin (SM). FIG. 10D shows induction of mouse TREM2-dependent luciferase reporter gene expression by the indicated amounts of full-length IgG1 isotype control antibody added in solution, and in conjunction with increasing concentrations of plate-bound sphingomyelin (SM). FIG. 10E shows induction of mouse TREM2-dependent luciferase reporter gene expression by full-length anti-TREM2 antibodies 2F6, 3A7, 3B10, 8F8, and 11H5, or the IgG1 isotype control added in solution, and in conjunction with increasing concentrations of plate-bound phosphatidylserine (PS). Results are expressed as absolute luminescence values. FIG. 10F shows induction of mouse TREM2-dependent luciferase reporter gene expression by full-length anti-TREM2 antibody 7E5 in comparison with a commercial antibody added in solution, and in conjunction with increasing concentrations of plate-bound sphingomyelin (SM). Mouse IgG1 and rat IgG2b antibodies were used as isotype controls.

FIG. 11A shows induction of human TREM2-dependent luciferase reporter gene expression by the indicated amounts of full-length anti-TREM2 antibody 9F5 added in solution, and in conjunction with increasing concentrations of plate-bound phosphatidylserine (PS). FIG. 11B shows induction of human TREM2-dependent luciferase reporter gene expression by the indicated amounts of full-length IgG1 isotype control antibody added in solution, and in conjunction with increasing concentrations of plate-bound phosphatidylserine (PS). FIG. 11C shows induction of human TREM2-dependent luciferase reporter gene expression by full-length anti-TREM2 antibodies 7B3, 9G1, 9G3, 9F5, and IgG1 isotype control antibody (msIgG1) in solution, and in conjunction with increasing concentrations of plate-bound phosphatidylserine (PS). Results are expressed as absolute luminescence values. FIG. 11D shows induction of human TREM2-dependent luciferase reporter gene expression by full-length anti-TREM2 antibodies 11A8, 12F9, 3B10, 8F8, and IgG1 isotype control antibody (msIgG1) in solution, and in conjunction with increasing concentrations of plate-bound phosphatidylserine (PS). Results are expressed as absolute luminescence values. FIG. 11E shows binding of a recombinant human TREM2 protein to APOE3 in the presence of 5 μg/ml of full-length anti-TREM2 antibodies 9F5, 7B3, and 9G3 and in the presence of IgG1 isotype control antibody (msIgG1) in solution. Average and SEM of two replicates are shown. FIG. 11F shows binding of a recombinant human TREM2 protein to APOE3 in the presence of 15 μg/ml of full-length anti-TREM2 antibodies 9F5, 7B3, and 9G3 and in the presence of IgG1 isotype control antibody (msIgG1) in solution. Average and SEM of two replicates are shown.

FIG. 12A shows the viability of wild-type (WT) bone marrow derived mouse macrophages after incubation with 100 nM soluble full-length anti-TREM2 antibodies 1H7, 2F6, 2H8, 3A7, 7E5, 7F8, and 8F8, or a commercial antibody (R&D Cat#F7E57291). As a negative control cells were incubated with mouse IgG1 and rat IgG2b isotype control antibodies. Results are expressed as % live cells, where 100% is the viability of untreated cells and 0% is the viability of cells cultured in the absence of the cytokine M-CSF. FIG. 12B shows the viability of wild-type (WT) bone marrow derived mouse macrophages after incubation with 2.5 ug/ml or 10 ug/ml of plate-bound full-length anti-TREM2 antibodies 2F6, 3A7, 7E5, and 8F8. As a negative control cells were incubated with mouse IgG1 (mIgG1). Results are expressed as luminescence, which is a measure of cell viability. The dotted line indicates the baseline average viability when cells are left untreated. FIG. 12C shows the number of immune cells expressing the markers CD11b or CD11b and Gr1 that are found in the brain of mice that have been injected with anti-TREM2 antibody 7E5 or an isotype control antibody (mIgG1).

FIG. 13A shows the design of an exemplary in vivo experiment to determine the effect of TREM2 antibodies injected into the abdominal cavity alone or in combination with LPS on the total number of immune cells. FIG. 13B shows the percentage of neutrophils in the abdominal cavity after injection of LPS, control (CTR) or TREM2 antibody 7E5 alone, or CTR or TREM2 antibody 7E5 in combination with LPS. FIG. 13C shows the number of neutrophil cells in the abdominal cavity after injection of LPS, control (CTR) or TREM2 antibody 7E5 alone, or CTR or TREM2 antibody 7E5 in combination with LPS. FIG. 13D shows the percentage of neutrophils in the abdominal cavity after injection of LPS, control (CTR) or TREM2 antibody 8F8 alone, or CTR or TREM2 antibody 8F8 in combination with LPS. FIG. 13E shows the number of neutrophil cells in the abdominal cavity after injection of LPS, control (CTR) or TREM2 antibody 8F8 alone, or CTR or TREM2 antibody 8F8 in combination with LPS. FIG. 13F shows the percentage of resident macrophages (CD11b⁺F4/80^{high}) in the abdominal cavity after injection of LPS, control (CTR) or TREM2 antibody 7E5 alone, or CTR or TREM2 antibody 7E5 in combination with LPS. FIG. 13G shows the number of resident macrophage cells (CD11b⁺F4/80^{high}) in the abdominal cavity after injection of LPS, control (CTR) or TREM2 antibody 7E5 alone, or CTR or TREM2 antibody 7E5 in combination with LPS. FIG. 13H shows the percentage of resident macrophages (CD11b⁺F4/80^{high}) in the abdominal cavity after injection of LPS, control (CTR) or TREM2 antibody 8F8 alone, or CTR or TREM2 antibody 8F8 in combination with LPS. FIG. 13I shows the number of resident macrophage cells (CD11b⁺F4/80^{high}) in the abdominal cavity after injection of LPS, control (CTR) or TREM2 antibody 8F8 alone, or CTR or TREM2 antibody 8F8 in combination with LPS. FIG. 13J shows the percentage of small infiltrating macrophages (CD11b⁺F4/80^{int}) in the abdominal cavity after injection of LPS, control (CTR) or TREM2 antibody 7E5 alone, or CTR or TREM2 antibody 7E5 in combination with LPS. FIG. 13K shows the number of small infiltrating macrophage cells (CD11b⁺F4/80^{int}) in the abdominal cavity after injection of LPS, control (CTR) or TREM2 antibody 7E5 alone, or CTR or TREM2 antibody 7E5 in combination with LPS. FIG. 13L shows the percentage of small infiltrating macrophages (CD11b⁺F4/80^{int}) in the abdominal cavity after injection of LPS, control (CTR) or TREM2 antibody 8F8 alone, or CTR or TREM2 antibody 8F8 in combination with LPS. FIG. 13M shows the number of small infiltrating macrophage cells (CD11b⁺F4/80^{int}) in the abdominal cavity after injection of LPS, control (CTR) or TREM2 antibody 8F8 alone, or CTR or TREM2 antibody 8F8 in combination with LPS. FIG. 13N shows the design of an exemplary in vivo experiment to determine the effect of TREM2 antibodies injected into the abdominal cavity alone or in combination with LPS on the production of inflammatory mediators CCL4, IL-1β, and MCP-1 (CCL2). FIG. 13O shows the concentration in pg/ml of CCL4 in the abdominal cavity after injection of control (CTR) or TREM2 antibodies 7E5 and 8F8 in combination with LPS. FIG. 13P shows the concentration in pg/ml of IL-1β in the abdominal cavity after injection of control (CTR) or TREM2 antibodies 7E5 and 8F8 in combination with LPS. FIG. 13Q shows the concentration in pg/ml of MCP-1 (CCL2) in the abdominal cavity after injection of control (CTR) or TREM2 antibodies 7E5 and 8F8 in combination with LPS.

FIG. 14 shows the average concentration (ug/ml) of 7E5 antibody found in blood serum at days 2, 4, 8, and 15 after injection of the indicated doses of antibody in the peritoneum of three mice. Measurement of soluble 7E5 antibody was done by standard ELISA. Data was analyzed with Prism6 software and fitted with exponential one-phase decay curve to calculate the half-life. The half-life of the antibodies is approximately 9.5 days in mouse serum.

FIG. 15 shows the concentration (ng/ml) of soluble TREM2 receptor (sTREM2) found in blood serum at days 2, 4, 8, and 15 after injection of the indicated doses of antibody in the peritoneum. Measurement of soluble TREM2 was done by ELISA.

FIG. 16A shows TREM2 receptor down regulation in culture in response to plate-bound phosphatidylserine (PS) and sphingomyelin (SM). FIG. 16 B shows TREM2 receptor down regulation in culture in response to soluble full-length anti-TREM2 antibodies 3A7 and 2F6 in solution, and in conjunction with increasing concentrations of plate-bound phosphatidylserine (PS).

FIG. 17A shows the change in the expression of pro-inflammatory and anti-inflammatory genes in the hippocampus of APP/PS1 mice that have been injected with anti-TREM2 antibody 7E5 using TaqMan assays containing TaqMan® gene expression probes for IL-1b, IL-6, TNFa, IL-12, YM-1, IL-1Ra, MRC1, IL-10, CD86, FCGR1B, and TGFb (Applied Biosystems, Invitrogen), and real-time PCR as described in Example 16. Fold change is relative to gene expression in control mice (dotted line). Treatment with anti-TREM2 antibody 7E5 significantly increased the expression of IL-1b, IL-6, TNFa, and CD86 by approximately 2-fold. The expression of FCGR1B was increased approximately 3-fold, and the expression of IL-10 was increased approximately 4-fold. By contrast, expression of IL-1Ra decreased by half. Expression of IL-12, YM-1, MRC1, and TGFB remained unchanged. All gene expression data was normalized to 18S rRNA expression. FIG. 17B shows the change in the expression of pro-inflammatory and anti-inflammatory genes in the hippocampus of 5XFAD mice 24 hours and 72 hours after mice were injected intracranially with anti-TREM2 antibody 7E5 using TaqMan assays containing TaqMan® gene expression probes for IL-1b, TNFa, YM-1, IL-1Rn CD86, TGF-β1, CCL2, CCL3, CCL5, CCR2, CXCL10, Gata3 and Rorc (Applied Biosystems, Invitrogen), and real-time PCR as described in Example 16. Fold change is relative to gene expression in mice treated with an isotype control antibody. The dotted line indicates the level of expression in mice treated with control antibody. Treatment with anti-TREM2 antibody 7E5 significantly increased the expression of IL-1b, TNFa, YM-1, CD86, CCL2, CCL3, CCR2, CXCL10, Gata3 and Rorc by approximately 2-fold 72 hours post injection. The expression of CCL5 was increased approximately 3-fold. Expression of IL-1Rn and TGFB instead remained unchanged. All gene expression data was normalized to 18S rRNA expression. *=Pval<0.05; **=Pval<0.01. FIG. 17C shows the change in the expression of FLT1 in the brain of APP/PS1 mice injected intracranially with 5 mg/ml 7E5 or control msIgG1 antibody. *Pval<0.01, Student's t-test. FIG. 17D-17P show expression of cytokines and chemokines in brains of 5XFAD mice 3 months after the mice were injected with 50 mg/kg anti-TREM2 antibody 7E5 weekly using TaqMan assays containing TaqMan® gene expression probes for CCL2, CXCL10, Rorc, TNFa, AXL, LDR, CXCR4, Fabp5, Fabp3, OPN, FLT1, CSF-1, and CD11c, and real-time PCR as described in Example 16. FIG. 17D shows results for CCL2. FIG. 17E shows results for CXCL10. FIG. 17F shows results for Rorc. FIG. 17G shows results for TNFa. FIG. 17H shows results for CSF-1. FIG. 17I shows results for OPN. FIG. 17J shows results for CD11c. FIG. 17K shows results for Flt1. FIG. 17L shows results for AXL. FIG. 17M shows results for LDR. FIG. 17N shows results for CXCR4. FIG. 17O shows results for Fabp5. FIG. 17P shows results for Fabp3. For FIG. 17D-17P *Pval<0.05, **Pval<0.01, ***Pval<0.001, One Way Anova with Tukey post hoc test. FIG. 17Q shows the quantification of Abeta peptide in the frontal cortex (FCX) and hippocampus (HPC) of APP/PS1 mice that were injected intracranially with anti-TREM2 antibody 7E5 or an isotype control antibody (mIgG1) using free-floating immunohistochemistry for Abeta stained with rabbit polyclonal antibody Aβ1-16 (Invitrogen) as described in Example 16. **=Pval<0.01, Two-way ANOVA with Fisher's PLSD post hoc test. FIG. 17R shows the quantification of Abeta peptide in the frontal cortex (FCX) and hippocampus (HPC) of 5XFAD mice that were chronically intraperitoneally injected with anti-TREM2 antibody 7E5 or an isotype control antibody (mIgG1) using free-floating immunohistochemistry for Abeta stained with rabbit polyclonal antibody Aβ1-16 (Invitrogen) as described in Example 16. *=Pval<0.05; **=Pval<0.01. FIG. 17S-17U show results from analysis of insoluble protein from frontal cortex of 5XFAD mice that have been chronically intraperitoneally injected with anti-TREM2 antibody 7E5 or an isotype control antibody (mIgG1) using Meso Scale Discovery Abeta kit that measures Abeta 38 (Ab38), Abeta 40 (Ab40), and Abeta 42 (Ab42). There is a significant decrease in insoluble Abeta42 after treatment with 7E5. FIG. 17S shows results with Abeta 38 (Ab38). FIG. 17T shows results with Abeta 40 (Ab40). FIG. 17U shows results with Abeta 42 (Ab42). FIG. 17V shows the quantification of CD11b expressing cells in the frontal cortex (FCX) and hippocampus (HPC) of APP/PS1 mice that were injected intracranially with anti-TREM2 antibody 7E5 or an isotype control antibody (mIgG1) using free-floating immunohistochemistry for CD11b stained with a rat monoclonal antibody (Serotec, Raleigh, N.C., USA) as described in Example 16. **=Pval<0.01. FIG. 17W shows the quantification of CD11b expressing cells in the frontal cortex (FCX) and hippocampus (HPC) of mice that were injected chronically intraperitoneally with anti-TREM2 antibody 7E5 or an isotype control antibody (mIgG1) using free-floating immunohistochemistry for CD11b stained with a rat monoclonal antibody (Serotec, Raleigh, N.C., USA) as described in Example 16. **=Pval<0.01. FIG. 17X shows cognitive function results assessed with the radial arm water maze test of WT or 5XFAD mice chronically injected with 7E5 or control antibody, as described in Example 16. The radial arm water maze test was performed after 12 weeks of treatment with antibodies. Graphs represent the average number of errors performed to complete the task. Blocks are the average of three trials. 5XFAD transgenic mice receiving the control antibody were significantly impaired compared to the non-transgenic wild-type mice (WT), scoring on average more than 3 errors throughout the second day of testing. By contrast, WT mice treated with either antibody scored less than one error in bocks 8 through 10, as expected from a mouse with normal cognitive function. 5XFAD transgenic mice that are treated with anti-TREM2 antibody 7E5 antibody performed significantly better than the control 5XFAD transgenic mice treated with the isotype antibody, and were indistinguishable from normal nontransgenic mice in blocks 5, 9, and 10, indicating recovery of cognitive functions. Bars indicate SEM, *=Pval<0.05, **=Pval<0.05. FIG. 17Y shows cognitive function results assessed with the novel object recognition test (NORT) of WT or 5xFAD mice chronically injected with 7E5 or control antibody, as described in Example 16. The NORT test was performed after 12 weeks of treatment with antibodies. Bar graphs represent the percentage of time spent at the new object. 5XFAD mice treated with the control antibody spent only ˜50% of the time exploring the novel object, which is indicative of highly unpaired cognitive function. By contrast, mice treated with the anti-TREM2 antibody 7E5 spent 67% of the time exploring the novel object, which is close to normal cognitive function, indicating almost full recovery. Post hoc Fisher's PLSD test was used for statistical analysis **=Pval<0.01.

FIG. 18 shows TREM2 expression on the indicated immune cell populations present in the spleen (SPL) or in the tumor (Tum) of naïve mice or mice bearing the indicated types of tumors.

FIG. 19A shows the tumor size in wild-type (WT) or TREM2-deficient (KO) mice, measured at day 8 or day 26 after inoculation with MC38 tumor cells. Each dot indicates an individual mouse. The mean and standard error (SEM) are indicated. Mann-Whitney U test was used for statistical analysis. FIG. 19B shows the median growth curve of MC38 cells implanted in wild-type (WT) or TREM2-deficient (TREM2 KO) mice.

FIG. 20 shows a dose dependent improvement in cognitive function in mice with traumatic brain injury that were treated with different doses of anti-TREM2 antibody 7E5. Cognitive function was assessed with the novel object recognition test (NORT), as described in Example 25. Treatment groups are: 1=40 mg/Kg 7E5; 2=20 mg/Kg 7E5; 3=10 mg/Kg 7E5; 4=5 mg/Kg 7E5 and CTR=40 mg/Kg isotype control antibody mIgG1. The NORT test was performed at day 32 after injury. Bar graphs represent the percentage of time spent at the new object from total exploration time spending of the two objects. “Baseline” bar graphs represent the time spent exploring two identical objects, which is similar regardless of the treatment that the mice have received. “Test” bar graphs represent the time spent exploring a new object. Mice with traumatic brain injury that are treated with the control antibody spent only 57.4±5.3% exploring the novel object, which is indicative of highly unpaired cognitive function. By contrast, mice that are treated with the highest dose of anti-TREM2 antibody 7E5 spent 73.9±5.4% of the time exploring the novel object, which is close to normal cognitive function, indicating almost full recovery. Post hoc Fisher's PLSD test was used for statistical analysis *=Pval<0.05.

FIG. 21A shows the amount of the cytokine TNFa measured in the peritoneal cavity of TREM2 wild-type mice (WT) and TREM2 knock-out mice (KO) that were injected with Brewer's Thioglyicollate and then administered anti-TREM2 antibody 7E5 or isotype control antibody (mIgG1). The concentration of TNFa increased about 6-fold in mice treated with antibody 7E5, as compared to control treated mice. FIG. 21B shows the amount of the cytokine CCL2 measured in the peritoneal cavity of TREM2 wild-type mice (WT) and TREM2 knock-out mice (KO) that were injected with Brewer's Thioglyicollate and then administered anti-TREM2 antibody 7E5 or isotype control antibody (mIgG1). The concentration of CCL2 increased of −2-fold in mice treated with antibody 7E5, as compared to control treated mice. The increase of these cytokines is specific because it does not occur in the TREM2 KO mice.

FIGS. 22A and 22B show results of Basso Mouse Scale (BMS) test to measure hindlimb performance in mice treated with anti-TREM2 antibody 7E5 (7E5) or isotype control antibody (Control IgG) after induction of spinal cord injury on day 0. FIG. 22A shows BMS scores. FIG. 22B shows BMS subscores. The results indicate that antibody 7E5 causes transient improvement in motor function after spinal cord contusion as measured by the BMS scoring system. *p<0.05, 2-way repeated ANOVA with Tukey post hoc test.

FIG. 23 shows percent (%) survival of human monocyte-derived dendritic cells after incubation with soluble TREM2 antibody 9F5 or 10A9. In contrast to antibody 10A9, there is no significant decrease in survival of dendritic cells upon incubation with antibody 9F5. “mIgG1” refers to a mouse istotype control antibody, and “Media” refers to a culture media-only control.

FIG. 24 shows that treatment of chronically dextran sodium sulfate (DSS)-challenged mice with the anti-TREM2 antibody 7E5 significantly reduces symptoms of chronic colitis. FIG. 24A shows body weight loss of chronically DSS-challenged mice treated with antibody 7E5. FIG. 24B shows disease activity index of chronically DSS-challenged mice treated with antibody 7E5. FIG. 24C shows colon length of chronically DSS-challenged mice treated with antibody 7E5. FIG. 24D shows colon endoscopic score of chronically DSS-challenged mice treated with antibody 7E5. Statistical analysis was performed using two-way ANOVA (FIGS. 24A and 24B) or unpaired t-test (FIGS. 24C and 24D), ***p<0.001, ****p<0.0001.

FIG. 25 shows that the anti-TREM2 antibody 9F5 can bind to and cross-link human TREM2 expressed by mouse macrophages. FIG. 25A shows FACS histograms demonstrating binding of human specific TREM2 antibodies 9F5 and 10A9 to human TREM2 expressed on macrophages from humanized TEM2 BAC transgenic mice (huTREM2 Tg), but not on macrophages from wild-type mice (WT). Anti-TREM2 antibodies that bind to both human and mouse TREM2 (antibody 2F5 and a commercial antibody from R&D) show positive binding to TREM2 expressed on macrophages from both from WT and huTREM2 Tg mice. Grey shaded plots are isotype stained cells, and black lined histograms show cells stained with anti-TREM2 antibodies. FIG. 25B shows secretion of TNFa by macrophages from humanized TEM2 BAC transgenic mice (Bac-Tg) that were stimulated in vitro with plate bound 9F5 or control antibody. FIG. 25C shows Dap12 phosphorylation (pTyr) after in vitro clustering of anti-TREM2 antibody 9F5 on macrophages from humanized TEM2 BAC transgenic mice (Bac-Tg) or wild-type mice (WT). The control Iantibody did not induce Dap12 phosphorylation.

FIG. 26A shows levels of soluble human Trem2 (sTREM2) measured in human TREM2 BAC transgenic mice (huTREM2 Tg) compared to wild-type mice (WT). Anti-TREM2 antibody T21-9 significantly increases plasma levels of sTREM2, while anti-TREM2 antibody 9F5 does not. FIG. 26B shows that anti-TREM2 antibody 9F5 binds only very weakly to sTREM2 in plasma samples, in contrast to anti-TREM2 antibody T21-9. The X axis denotes the dilution factor of the plasma tested and the Y axis shows the optical density readout.

### DETAILED DESCRIPTION OF THE PRESENT DISCLOSURE

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### Definitions

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “therapeutically effective amount” is at least the minimum concentration required to effect a measurable improvement of a particular disease, disorder, or condition. A therapeutically effective amount herein may vary according to factors such as the disease state, age, sex, and weight of the patient, and the ability of the anti-TREM2 antibody to elicit a desired response in the individual. A therapeutically effective amount is also one in which any toxic or detrimental effects of the anti-TREM2 antibody are outweighed by the therapeutically beneficial effects.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Ten and Tristram G. Parslow (eds.), Appleton & Lange, Norwalk, Conn., 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” antibody, such as an isolated anti-TREM2 antibody of the present disclosure, is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T-cells since at least one component of the antibody's natural environment will not be present. Ordinarily, however, an isolated polypeptide or antibody will be prepared by at least one purification step.

The “variable region” or “variable domain” of an antibody, such as an anti-TREM2 antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-TREM2 antibodies of the present disclosure. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as a monoclonal anti-TREM2 antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” or “whole antibody” are used interchangeably to refer to an antibody, such as an anti-TREM2 antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-TREM2 antibodies of the present disclosure, produces two identical antigen-binding fragments, called “Fab” fragments, and a residual “Fe” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding. For a review of the sFv, see Plückthun in The Pharmacology of Monoclonal Antibodies, vol. 113, Rosenburg and Moore eds., Springer-VerLAG-3, New York, pp. 269-315 (1994).

“Functional fragments” of antibodies, such as anti-TREM2 antibodies of the present disclosure, comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Nat'l Acad. Sci. USA 90:6444-48 (1993).

As used herein, a “chimeric antibody” refers to an antibody (immunoglobulin), such as a chimeric anti-TREM2 antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-TREM2 antibodies of the present disclosure, are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody, such as an anti-TREM2 antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e g, immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain, such as that of an anti-TREM2 antibody of the present disclosure, that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The phrase “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU or, Kabat numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. References to residue numbers in the variable domain of antibodies means residue numbering by the Kabat numbering system. References to residue numbers in the constant domain of antibodies means residue numbering by the EU or, Kabat numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferable those changes occur at only three, two, or one of positions 71H, 73H and 78H; for instance, the amino acid residues at those positions may by 71A, 73T and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position, e.g., of an anti-TREM2 antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody, such as an affinity matured anti-TREM2 antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody, such as between an anti-TREM2 antibody and TREM2 that is determinative of the presence of the target in the presence of a heterogeneous population of molecules including biological molecules. For example, an antibody, such as an anti-TREM2 antibody of the present disclosure, that specifically or preferentially binds to a target or an epitope is an antibody that binds this target or epitope with greater affinity, avidity, more readily, and/or with greater duration than it binds to other targets or other epitopes of the target. It is also understood by reading this definition that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 10⁸ M⁻¹ to 10⁹ M⁻¹, or about 10¹⁰ M⁻¹ to 10¹¹M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, an “interaction” between a TREM2 protein, or DAP12 protein, and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

An “agonist” antibody or an “activating” antibody is an antibody, such as an agonist anti-TREM2 antibody of the present disclosure, that induces (e.g., increases) one or more activities or functions of the antigen after the antibody binds the antigen.

An “antagonist” antibody or a “blocking” antibody is an antibody, such as an antagonist anti-TREM2 antibody of the present disclosure, that reduces or eliminates (e.g., decreases) antigen binding to one or more ligand after the antibody binds the antigen, and/or that reduces or eliminates (e.g., decreases) one or more activities or functions of the antigen after the antibody binds the antigen. In some embodiments, antagonist antibodies, or blocking antibodies substantially or completely inhibit antigen binding to one or more ligand and/or one or more activities or functions of the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU or, Kabat numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of antibodies.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

An “isolated” nucleic acid molecule encoding an antibody, such as an anti-TREM2 antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### Overview

The present disclosure relates to anti-TREM2 antibodies (e.g., monoclonal antibodies) with one or more agonist or antagonist activities; methods of making and using such antibodies; pharmaceutical compositions containing such antibodies; nucleic acids encoding such antibodies; and host cells containing nucleic acids encoding such antibodies.

In some embodiments, the agonistic activities of the anti-TREM2 antibodies of the present disclosure are due, at least in part, to the ability of the antibodies to enhance one or more TREM2 activities induced by binding of one or more TREM2 ligands to the TREM2 protein without competing with or otherwise blocking binding of the one or more TREM2 ligands to the TREM2 protein. hi some embodiments, the enhancement of the one or more TREM2 activities by the anti-TREM2 antibodies is compared to the one or more TREM2 activities induced by binding of the one or more TREM2 ligands to the TREM2 protein in the absence of the anti-TREM2 antibodies. In some embodiments, enhancement of one or more TREM2 activities can be determined or tested in vitro or in vivo by any of several techniques disclosed herein (see, e.g., Examples 3-13 and 24).

Accordingly, certain aspects of the present disclosure are based, at least in part, on the identification of anti-TREM2 antibodies that are capable of binding to both human and mouse TREM2 with high affinity (see, e.g., Example 1); that can activate and enhance (e.g., by synergizing with TREM2 ligands) TREM2 activities (see, e.g., Examples 3-13 and 24). Advantageously, agonist anti-TREM2 antibodies of the present disclosure was shown to be therapeutically effective in treating Alzheimer's disease and symptoms of Alzheimer's disease in several mouse models of Alzheimer's disease (see, e.g., Example 16).

Further aspects of the present disclosure are based, at least in part, on the surprising discovery that the anti-TREM2 antibodies of the present disclosure can also induce antagonistic activities when the antibody is produced or otherwise formatted such that it is incapable of inducing or retaining TREM2 receptor clustering. In some embodiments, anti-TREM2 antibodies of the present disclosure exhibit one or more antagonistic TREM2 activities, including, without limitation, inhibition of TREM2-dependent gene activation (see, e.g., Examples 7 and 8).

### TREM2 Proteins

In one aspect, the present disclosure provides antibodies that bind to a TREM2 protein of the present disclosure and induce one or more TREM2 activities and/or enhance one or more TREM2 activities after binding to a TREM2 protein expressed in a cell.

TREM2 proteins of the present disclosure include, without limitation, a human TREM2 protein (Uniprot Accession No. Q9NZC2; SEQ ID NO: 1), and a non-human mammalian TREM2 protein, such as mouse TREM2 protein (Uniprot Accession No. Q99NH8; SEQ ID NO: 2), rat TREM2 protein (Uniprot Accession No. D3ZZ89; SEQ ID NO: 3), Rhesus monkey TREM2 protein (Uniprot Accession No. F6QVF2; SEQ ID NO: 4), bovine TREM2 protein (Uniprot Accession No. Q05B59; SEQ ID NO: 5), equine TREM2 protein (Uniprot Accession No. F7D6LO; SEQ ID NO: 6), pig TREM2 protein (Uniprot Accession No. H2EZZ3; SEQ ID NO: 7), and dog TREM2 protein (Uniprot Accession No. E2RP46; SEQ ID NO: 8). As used herein “TREM2 protein” refers to both wild-type sequences and naturally occurring variant sequences.

Triggering receptor expressed on myeloid cells-2 (TREM2) is variously referred to as TREM-2, TREM2a, TREM2b, TREM2c, triggering receptor expressed on myeloid cells-2a, and triggering receptor expressed on monocytes-2. TREM2 is a 230 amino acid membrane protein. TREM2 is an immunoglobulin-like receptor primarily expressed on myeloid lineage cells, including without limitation, macrophages, dendritic cells, monocytes, Langerhans cells of skin, Kupffer cells, osteoclasts, and microglia. In some embodiments, TREM2 forms a receptor signaling complex with DAP12. In some embodiments, TREM2 phosphorylates and signals through DAP12 (an ITAM domain adaptor protein). In some embodiments TREM2 signaling results in the downstream activation of PI3K or other intracellular signals. On Myeloid cells, Toll-like receptor (TLR) signals are important for the activation of TREM2 activities, e.g., in the context of an infection response. TLRs also play a key role in the pathological inflammatory response, e.g., TLRs expressed in macrophages and dendritic cells.

In some embodiments, an example of a human TREM2 amino acid sequence is set forth below as SEQ ID NO: 1:

```
                10 20 30 40
MEPLRLLILL FVTELSGAHN TTVFQGVAGQ SLQVSCPYDS
                50 60 70 80
MKHWGRRKAW CRQLGEKGPC QRVVSTHNLW LLSFLRRWNG
                90 100 110 120
STAITDDTLG GTLTITLRNL QPHDAGLYQC QSLHGSEADT
              130 140 150 160
LRKVLVEVLA DPLDHRDAGD LWFPGESESF EDAHVEHSIS
              170 180 190 200
RSLLEGEIPF PPTSILLLLA CIFLIKILAA SALWAAAWHG
              210 220 230
QKPGTHPPSE LDCGHDPGYQ LQTLPGLRDT
```

In some embodiments, the human TREM2 is a preprotein that includes a signal peptide. In some embodiments, the human TREM2 is a mature protein. In some embodiments, the mature TREM2 protein does not include a signal peptide. In some embodiments, the mature TREM2 protein is expressed on a cell. In some embodiments, TREM2 contains a signal peptide located at amino acid residues 1-18 of human TREM2 (SEQ ID NO: 1); an extracellular immunoglobulin-like variable-type (IgV) domain located at amino acid residues 29-112 of human TREM2 (SEQ ID NO: 1); additional extracellular sequences located at amino acid residues 113-174 of human TREM2 (SEQ ID NO: 1); a transmembrane domain located at amino acid residues 175-195 of human TREM2 (SEQ ID NO: 1); and an intracellular domain located at amino acid residues 196-230 of human TREM2 (SEQ ID NO: 1).

The transmembrane domain of human TREM2 contains a lysine at amino acid residue 186 that can interact with an aspartic acid in DAP12, which is a key adaptor protein that transduces signaling from TREM2, TREM1, and other related IgV family members.

Homologues of human TREM2 include, without limitation, the natural killer (NK) cell receptor NK-p44 (NCTR2), the polymeric immunoglobulin receptor (pIgR), CD300E, CD300A, CD300C, and TREML1/TLT1. In some embodiments, NCTR2 has similarity with TREM2 within the IgV domain.

### DAP12 Proteins

In one aspect, the present disclosure provides antibodies that may further bind to a DAP12 protein of the present disclosure and modulate one or more DAP12 activities after binding to a DAP12 protein expressed in a cell.

DAP12 proteins of the present disclosure include, without limitation, a mammalian (e.g., non-human mammal) DAP12 protein, human DAP12 protein (Uniprot Accession No. 043914), mouse DAP12 protein (Uniprot Accession No. 054885), rat DAP12 protein (Uniprot Accession No. Q6X9T7), Rhesus monkey DAP12 protein (Uniprot Accession No. Q8WNQ8), bovine DAP12 protein (Uniprot Accession No. Q95J80), and pig DAP12 protein (Uniprot Accession No. Q9TU45). As used herein “DAP12 protein” refers to both wild-type sequences and naturally occurring variant sequences.

DNAX-activation protein 12 (DAP12) is variously referred to as Killer-activating receptor-associated protein, KAR-associated protein (KARAP), PLOSL, PLO-SL, TYRO protein, and tyrosine kinase-binding protein. DAP12 is a 113 amino acid membrane protein. In some embodiments, DAP12 functions as a transmembrane signaling polypeptide, which contains an immunoreceptor tyrosine-based activation motif (ITAM) in its cytoplasmic domain. It may associate with the killer-cell inhibitory receptor (KIR) family of membrane glycoproteins and may act as an activating signal transduction element. In other embodiments, the DAP12 protein may bind zeta-chain (TCR) associated protein kinase 70 kDa (ZAP-70) and spleen tyrosine kinase (SYK), and play a role in signal transduction, bone modeling, brain myelination, and inflammation.

Mutations within the DAP12-encoding gene have been associated with polycystic lipomembranous osteodysplasia with sclerosing leukoencephalopathy (PLOSL), also known as Nasu-Hakola disease. Without wishing to be bound by theory, it is believed that the DAP12 receptor is TREM2, which also causes PLOSL. Multiple alternative transcript variants encoding distinct isoforms of DAP12 have been identified. DAP12 non-covalently associates with activating receptors of the CD300 family. Cross-linking of CD300-TYROBP/DAP12 complexes results in cellular activation, such as neutrophil activation mediated by integrin. DAP12 is a homodimer; disulfide-linked protein. In some embodiments, DAP12 interacts with SIRPB1, TREM1, CLECSFS, SIGLEC14, CD300LB, CD300E, and CD300D by similarity and via ITAM domain, as well as with SYK via SH2 domain. In other embodiments, DAP12 activates SYK, which mediates neutrophils and macrophages integrin-mediated activation. In other embodiments, DAP12 interacts with KLRC2 and KIR2DS3.

In some embodiments, an example of a human DAP12 amino acid sequence is set forth below as SEQ ID NO: 887:

```
                10 20 30 40
MGGLEPCSRL LLLPLLLAVS GLRPVQAQAQ SDCSCSTVSP
                50 60 70 80
GVLAGIVMGD LVLTVLIALA VYFLGRLVPR GRGAAEAATR
                90 100 110
KQRITETESP YQELQGQRSD VYSDLNTQRPYYK
```

In some embodiments, the human DAP12 is a preprotein that includes a signal peptide. In some embodiments, the human DAP12 is a mature protein. In some embodiments, the mature DAP12 protein does not include a signal peptide. In some embodiments, the mature DAP12 protein is expressed on a cell. DAP12 is a single-pass type I membrane protein. It contains an extracellular domain located at amino acid residues 22-40 of human DAP12 (SEQ ID NO: 887); a transmembrane domain located at amino acid residues 41-61 of human DAP12 (SEQ ID NO: 887); and an intracellular domain located at amino acid residues 62-113 of human DAP12 (SEQ ID NO: 887). The immunoreceptor tyrosine-based activation motif (ITAM) domain is located at amino acid residues 80-118 of human DAP12 (SEQ ID NO: 887).

In some embodiments, an aspartic acid residue in DAP12 interacts with the transmembrane domain of human TREM2 containing a lysine at amino acid residue 186, and transduces signaling from TREM2, TREM1, and other related IgV family member proteins.

### Anti-TREM2 Antibodies

Certain aspects of the present disclosure relate to antibodies (e.g., monoclonal antibodies) that specifically bind to TREM2. In some embodiments, antibodies of the present disclosure bind a mature TREM2 protein. In some embodiments, antibodies of the present disclosure bind a mature TREM2 protein, wherein the mature TREM2 protein is expressed on a cell. In some embodiments, antibodies of the present disclosure bind a TREM2 protein expressed on one or more human cells selected from human dendritic cells, human macrophages, human monocytes, human osteoclasts, human Langerhans cells of skin, human Kupffer cells, human microglia, and any combinations thereof. In some embodiments, antibodies of the present disclosure are agonist antibodies. In some embodiments, antibodies of the present disclosure are inert antibodies. In some embodiments, antibodies of the present disclosure are antagonist antibodies.

In some embodiments, anti-TREM2 antibodies of the present disclosure bind to a TREM2 protein without competing with, inhibiting, or otherwise blocking one or more TREM2 ligands from binding to the TREM2 protein. Examples of suitable TREM2 ligands include, without limitation, TREM2 ligands expressed by E. coli cells, apoptotic cells, nucleic acids, anionic lipids, APOE, APOE2, APOE3, APOE4, anionic APOE, anionic APOE2, anionic APOE3, anionic APOE4, lipidated APOE, lipidated APOE2, lipidated APOE3, lipidated APOE4, zwitterionic lipids, negatively charged phospholipids, phosphatidylserine, sulfatides, phosphatidylcholin, sphingomyelin, membrane phospholipids, lipidated proteins, proteolipids, lipidated peptides, and lipidated amyloid beta peptide. Accordingly, in certain embodiments, the one or more TREM2 ligands comprise E. coli cells, apoptotic cells, nucleic acids, anionic lipids, zwitterionic lipids, negatively charged phospholipids, phosphatidylserine (PS), sulfatides, phosphatidylcholin, sphingomyelin (SM), phospholipids, lipidated proteins, proteolipids, lipidated peptides, and lipidated amyloid beta peptide.

In some embodiments, anti-TREM2 antibodies of the present disclosure do not inhibit the growth of one or more innate immune cells. In some embodiments, anti-TREM2 antibodies of the present disclosure bind to one or more primary immune cells with a K_{D} of less than 50 nM, less than 45 nM, less than 40 nM, less than 35 nM, less than 30 nM, less than 25 nM, less than 20 nM, less than 15 nM, less than 10 nM, less than 9 nM, less than 8 nM, less than 7 nM, less than 6 nM, less than 5 nM, less than 4 nM, less than 3 nM, less than 2 nM, or less than 1 nM. In some embodiments, an anti-TREM2 antibody of the present disclosure accumulates in the brain, or the cerebrospinal fluid (CSF), or both to an extent that is 1% or more, 2% or more, 3% or more, 4% or more, 5% or more, 6% or more, 7% or more, 8% or more, 9% or more, 10% or more of the concentration of the antibody in the blood. In some embodiments, the dissociation constant (K_{D}) is determined at a temperature of approximately 4° C. In some embodiments, the K_{D} is determined using a monovalent antibody (e.g., a Fab) or a full-length antibody in a monovalent form. Methods for the preparation and selection of antibodies that interact and/or bind with specificity to TREM2 are described herein. (e.g., see Example 1).

Agonist Anti-TREM2 Antibodies

Anti-TREM2 antibodies of the present disclosure generally bind to one or more TREM2 proteins expressed on a cell. One class of antibodies is agonist antibodies. For example, the TREM2 receptor is thought to require clustering on the cell surface in order to transduce a signal. Thus agonist antibodies may have unique features to stimulate, for example, the TREM2 receptor. For example, they may have the correct epitope specificity that is compatible with receptor activation, as well as the ability to induce or retain receptor clustering on the cell surface. In addition, agonist anti-TREM2 antibodies of the present disclosure may display the ability to bind TREM2 without blocking simultaneous binding of one or more TREM2 ligands. The anti-TREM2 antibodies of the present disclosure may further display additive and/or synergistic functional interactions with one or more TREM2 ligands. Thus, in some embodiments, the maximal activity of TREM2 when bound to anti-TREM2 antibodies of the present disclosure in combination with one or more TREM2 ligands of the present disclosure may be greater (e.g., enhanced) than the maximal activity of TREM2 when exposed to saturating concentrations of ligand alone or to saturating concentrations of the antibody alone. In addition, the activity of TREM2 at a given concentration of TREM2 ligand may be greater (e.g., enhanced) in the presence of the antibody. Accordingly, in some embodiments, anti-TREM2 antibodies of the present disclosure have an additive effect with the one or more TREM2 ligands to enhance the one or more TREM2 activities when bound to the TREM2 protein. In some embodiments, anti-TREM2 antibodies of the present disclosure synergize with the one or more TREM2 ligands to enhance the one or more TREM2 activities. In some embodiments, anti-TREM2 antibodies of the present disclosure increase the potency of the one or more TREM2 ligands to induce the one or more TREM2 activities, as compared to the potency of the one or more TREM2 ligands to induce the one or more TREM2 activities in the absence of the antibody. In some embodiments, anti-TREM2 antibodies of the present disclosure enhance the one or more TREM2 activities in the absence of cell surface clustering of TREM2. In some embodiments, anti-TREM2 antibodies of the present disclosure enhance the one or more TREM2 activities by inducing or retaining cell surface clustering of TREM2. In some embodiments, anti-TREM2 antibodies of the present disclosure are clustered by one or more Fc-gamma receptors expressed on one or more immune cells, including without limitation, B cells and microglial cells. In some embodiments, enhancement of the one or more TREM2 activities induced by binding of one or more TREM2 ligands to the TREM2 protein is measured on primary cells, including without limitation, dendritic cells, bone marrow-derived dendritic cells, monocytes, microglia, macrophages, neutrophils, NK cells, osteoclasts, Langerhans cells of skin, and Kupffer cells, or on cell lines, and the enhancement of the one or more TREM2 activities induced by binding of one or more TREM2 ligands to the TREM2 protein is measured, for example, utilizing an in vitro cell assay.

In vivo, anti-TREM2 antibodies of the present disclosure may activate receptors by multiple potential mechanisms. In some embodiments, agonistic anti-TREM2 antibodies of the present disclosure, have, due to the correct epitope specificity, the ability to activate TREM2 in solution without having to be clustered with a secondary antibody, bound on plates, or clustered through Fcg receptors. In some embodiments, anti-TREM2 antibodies of the present disclosure have isotypes of human antibodies, such as IgG2, that have, due to their unique structure, an intrinsic ability to cluster receptors or retain receptors in a clustered configuration, thereby activating receptors such as TREM2 without binding to an Fc receptor (e.g., White et al., (2015) Cancer Cell 27, 138-148).

In some embodiments, anti-TREM2 antibodies of the present disclosure cluster receptors (e.g., TREM2) by binding to Fcg receptors on adjacent cells. Binding of the constant IgG Fc part of the antibody to Fcg receptors leads to aggregation of the antibodies, and the antibodies in turn aggregate the receptors to which they bind through their variable region (Chu et al (2008) Mol Immunol, 45:3926-3933; and Wilson et al., (2011) Cancer Cell 19, 101-113). Binding to the inhibitory Fcg receptor FcgR (FcgRIIB) that does not elicit cytokine secretion, oxidative burst, increased phagocytosis, and enhanced antibody-dependent, cell-mediated cytotoxicity (ADCC) is often a preferred way to cluster antibodies in vivo, since binding to FcgRIIB is not associated with immune adverse effects. Any suitable assay described herein (see, e.g., Example 4) may be used to determine antibody clustering.

Other mechanisms may also be used to cluster receptors (e.g., TREM2). For example, in some embodiments, antibody fragments (e.g., Fab fragments) that are cross-linked together may be used to cluster receptors (e.g., TREM2) in a manner similar to antibodies with Fc regions that bind Fcg receptors, as described above. In some embodiments, cross-linked antibody fragments (e.g., Fab fragments) may function as agonist antibodies if they induce receptor clustering on the cell surface and bind an appropriate epitope on the target (e.g., TREM2).

In some embodiments, antibodies of the present disclosure that bind a TREM2 protein may include agonist antibodies that due to their epitope specificity bind TREM2 and activate one or more TREM2 activities. In some embodiments, such antibodies may bind to the ligand-binding site on TREM2 and mimic the action of one or more TREM2 ligands, or stimulate the target antigen to transduce signal by binding to one or more domains that are not the ligand-binding sites. In some embodiments, the antibodies do not compete with or otherwise block ligand binding to TREM2. In some embodiments, the antibodies, act additively or synergistically with one or more TREM2 ligands to activate and/or enhance one more TREM2 activities.

In some embodiments, TREM2 activities that may be induced and/or enhanced by anti-TREM2 antibodies of the present disclosure and/or one or more TREM2 ligands of the present disclosure include, without limitation, TREM2 binding to DAP12; TREM2 phosphorylation; DAP12 phosphorylation; activation of one or more tyrosine kinases, optionally where the one or more tyrosine kinases comprise a Syk kinase, ZAP70 kinase, or both; activation of phosphatidylinositol 3-kinase (PI3K); activation of protein kinase B (Akt); recruitment of phospholipase C-gamma (PLC-gamma) to a cellular plasma membrane, activation of PLC-gamma, or both; recruitment of TEC-family kinase dVav to a cellular plasma membrane; activation of nuclear factor-rB (NF-rB); inhibition of MAPK signaling; phosphorylation of linker for activation of T cells (LAT), linker for activation of B cells (LAB), or both; activation of IL-2-induced tyrosine kinase (Itk); modulation of one or more pro-inflammatory mediators selected from IFN-β, IL-1α, IL-1β, TNF-α, YM-1, IL-6, IL-8, CRP, CD86, MCP-1/CCL2, CCL3, CCL4, CCL5, CCR2, CXCL-10, Gata3, Rorc, IL-20 family members, IL-33, LIF, IFN-gamma, OSM, CNTF, GM-CSF, CSF-1, MHC-II, OPN, CD11c, GM-CSF, IL-11, IL-12, IL-17, IL-18, and IL-23, optionally where the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; modulation of one or more anti-inflammatory mediators selected from IL-4, IL-10 TGF-β, IL-13, IL-35 IL-16, IFN-alpha, IL-1Ra, VEGF, G-CSF, YM, AXL, FLT1, and soluble receptors for TNF or IL-6, optionally wherein the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; modulation of one or more genes whose expression is increased upon induction of inflammation, optionally wherein the one or more genes are selected from Fabp3, Fabp5, and LDR; phosphorylation of extracellular signal-regulated kinase (ERK); modulation of one or more genes whose expression is increased upon induction of inflammation, optionally wherein the one or more genes are selected from the group consisting of Fabp3, Fabp5, and LDR; modulated expression of C-C chemokine receptor 7 (CCR7) in one or more cells selected from macrophages, M1 macrophages, activated M1 macrophages, M2 macrophages, dendritic cells, monocytes, osteoclasts, Langerhans cells of skin, Kupffer cells, microglia, M1 microglia, activated M1 microglia, and M2 microglia, and any combination thereof; induction of microglial cell chemotaxis toward CCL19 and CCL21 expressing cells; normalization of disrupted TREM2/DAP12-dependent gene expression; recruitment of Syk, ZAP70, or both to a DAP12/TREM2 complex; increasing activity of one or more TREM2-dependent genes, optionally where the one or more TREM2-dependent genes comprise nuclear factor of activated T-cells (NFAT) transcription factors; increased maturation of dendritic cells, monocytes, microglia, M1 microglia, activated M1 microglia, and M2 microglia, macrophages, M1 macrophages, activated M1 macrophages, M2 macrophages, or any combination thereof; increased ability of dendritic cells, monocytes, microglia, M1 microglia, activated M1 microglia, and M2 microglia, macrophages, M1 macrophages, activated M1 macrophages, M2 macrophages, or any combination thereof to prime or modulate the function of T cells, optionally wherein the T cells are one or more cells selected from CD8+ T cells, CD4+T cells regulatory T cells, and any combination thereof; enhanced ability, normalized ability, or both of bone marrow-derived dendritic cells to prime or modulate function of antigen-specific T cells, optionally wherein the antigen-specific T cells are one or more cells selected from CD8+ T cells, CD4+T cells regulatory T cells, and any combination thereof; induction of osteoclast production, increased rate of osteoclastogenesis, or both; increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; increasing the function of dendritic cells, macrophages, M1 macrophages, activated M1 macrophages, M2 macrophages, microglia, M1 microglia, activated M1 microglia, and M2 microglia, or any combination thereof; increasing phagocytosis by dendritic cells, macrophages, M1 macrophages, activated M1 macrophages, M2 macrophages, monocytes, microglia, M1 microglia, activated M1 microglia, and M2 microglia, or any combination thereof; induction of one or more types of clearance selected from apoptotic neuron clearance, nerve tissue debris clearance, non-nerve tissue debris clearance, bacteria or other foreign body clearance, disease-causing agent clearance, tumor cell clearance, or any combination thereof, optionally where the disease-causing agent is selected from amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products including DiPeptide Repeats, (DPRs peptides) composed of glycine-alanine (GA), glycine-proline (GP), glycine-arginine (GR), proline-alanine (PA), or proline-arginine (PR), antisense GGCCCC (G2C4) repeat-expansion RNA; induction of phagocytosis of one or more of apoptotic neurons, nerve tissue debris, non-nerve tissue debris, bacteria, other foreign bodies, disease-causing agents, tumor cells, or any combination thereof, optionally where the disease-causing agent is selected from amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products including DiPeptide Repeats, (DPRs peptides) composed of glycine-alanine (GA), glycine-proline (GP), glycine-arginine (GR), proline-alanine (PA), or proline-arginine (PR), antisense GGCCCC (G2C4) repeat-expansion RNA; modulated expression of one or more stimulatory molecules selected from CD83, CD86 MHC class II, CD40, and any combination thereof, optionally where the CD40 is expressed on dendritic cells, monocytes, macrophages, or any combination thereof, and optionally where the dendritic cells comprise bone marrow-derived dendritic cells; modulating secretion of one or more pro-inflammatory mediators, optionally where the one or more inflammatory mediators are selected from IFN-β, IL-1α, IL-1β, CD86, TNF-α, IL-6, IL-8, CRP, MCP-1/CCL2, CCL3, CCL4, CCL5, CCR2, CXCL-10, Gata3, IL-20 family members, IL-33, LIF, IFN-gamma, OSM, CNTF, CSF1, OPN, CD11c, GM-CSF, IL-11, IL-12, IL-17, IL-18, and IL-23, and any combination thereof; modulation of one or more anti-inflammatory mediators selected from the group consisting of IL-4, IL-10 TGF-β, IL-13, IL-35 IL-16, IFN-alpha, IL-1Ra, VEGF, G-CSF, YM, AXL, FLT1, and soluble receptors for TNF or IL-6, and any combination thereof; modulating expression of one or more proteins selected from C1qa, C1qB, C1qC, C1s, C1R, C4, C2, C3, ITGB2, HMOX1, LAT2. CASP1, CSTA, VSIG4, MS4A4A, C3AR1, GPX1, TyroBP, ALOXSAP, ITGAM, SLC7A7, CD4, ITGAX, PYCARD, and VEGF; increasing memory; and reducing cognitive deficit. In some embodiments, anti-TREM2 antibodies of the present disclosure increase memory and/or reduce cognitive deficit when administered to an individual.

As used herein, an anti-TREM2 antibody of the present disclosure enhances one or more TREM2 activities induced by binding of one or more TREM2 ligands to the TREM2 protein if it induces at least a 2-fold, at least a 3-fold, at least a 4-fold, at least a 5-fold, at least a 6-fold, at least a 7-fold, at least a 8-fold, at least a 9-fold, at least a 10-fold, at least an 11-fold, at least a 12-fold, at least a 13-fold, at least a 14-fold, at least a 15-fold, at least a 16-fold, at least a 17-fold, at least an 18-fold, at least a 19-fold, at least a 20-fold or greater increase in the one or more TREM2 activities as compared to levels of the one or more TREM2 activities induced by binding of one or more TREM2 ligands to the TREM2 protein in the absence of the anti-TREM2 antibody. In some embodiments, the increase in one more TEM2 activities may be measured by any suitable in vitro cell-based assays or suitable in vivo model described herein or known in the art, for example, by utilizing a luciferase-based reporter assay to measure TREM2-dependent gene expression, using Western blot analysis to measure increase in TREM2-induced phosphorylation of downstream signaling partners, such as Syk, or by utilizing flow cytometry, such as fluorescence-activated cell sorting (FACS) to measure changes in cell surface levels of markers of TREM2 activation. Any in vitro cell-based assays or suitable in vivo model described herein or known in the art may be used to measure interaction (e.g., binding) between TREM2 and one or more TREM2 ligands.

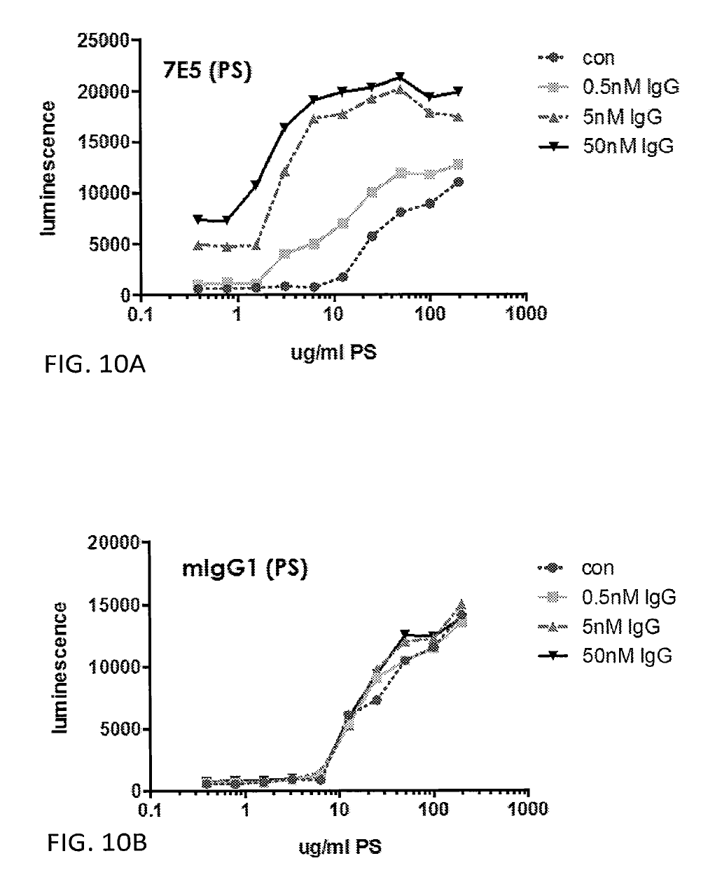

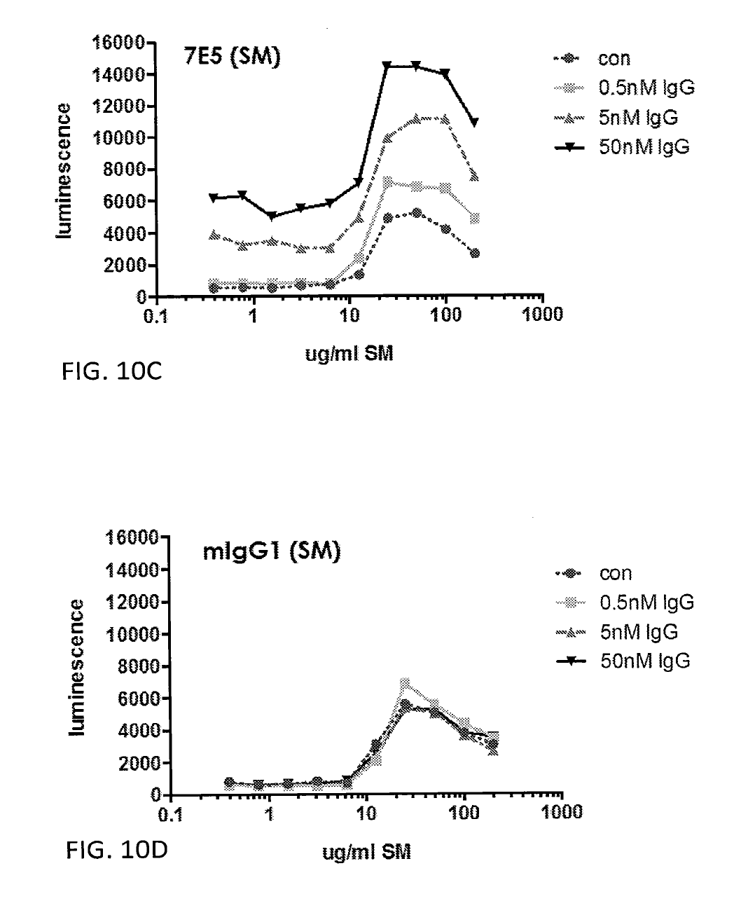

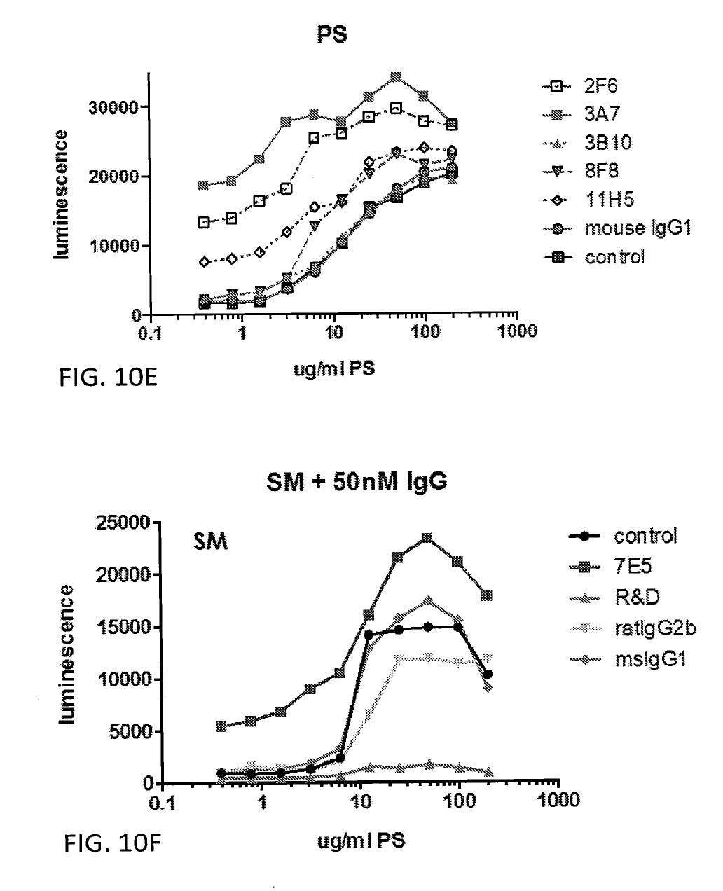

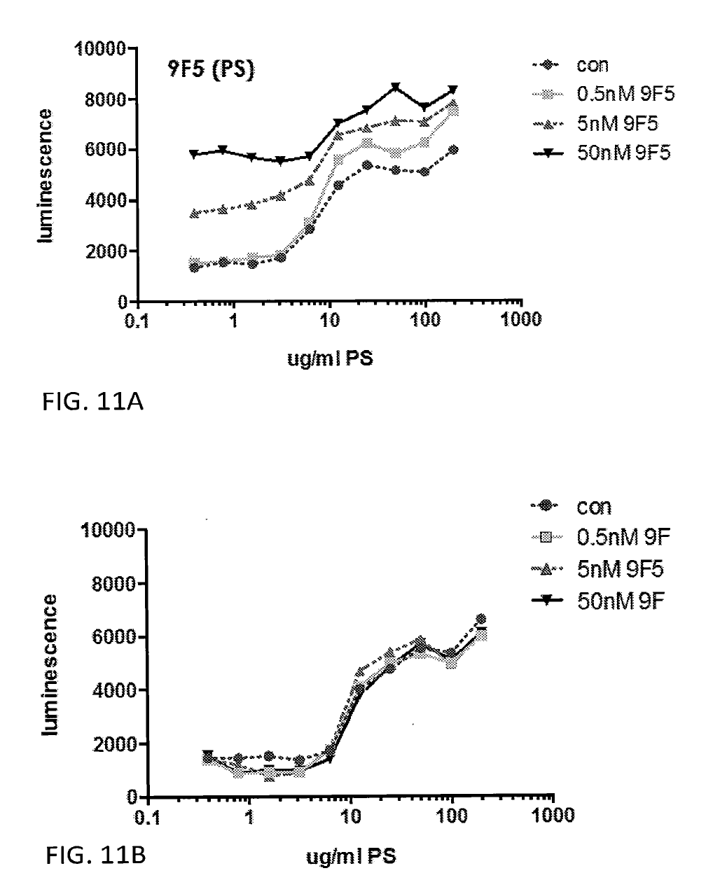

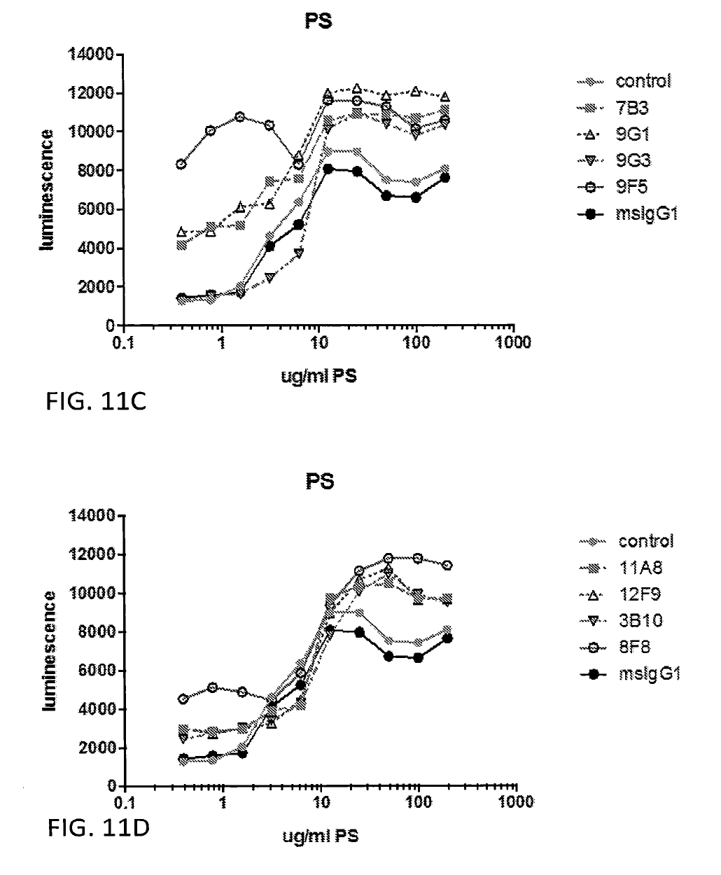

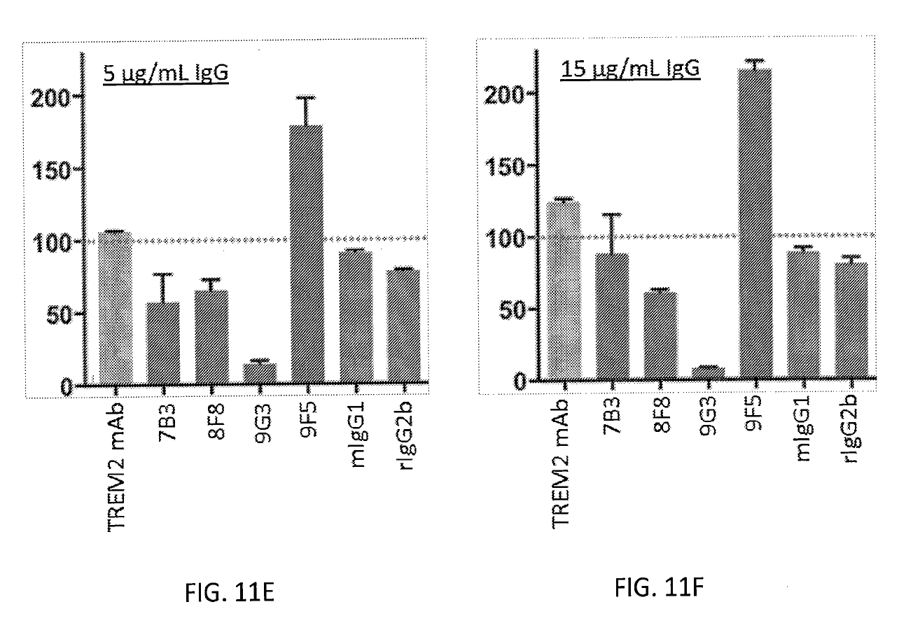

In some embodiments an anti-TREM2 antibody of the present disclosure enhances one or more TREM2 activities induced by binding of a TREM2 ligand to the TREM2 protein if it induces an increase that ranges from about 1-fold to about 6-fold, or more than 6-fold in ligand-induced TREM2-dependent gene transcription when used at a concentration that ranges from about 0.5 nM to about 50 nM, or greater than 50 nM, and as compared to the level of TREM2-dependent gene transcription induced by binding of the TREM2 ligand to the TREM2 protein in the absence of the anti-TREM2 antibody when the TREM2 ligand is used at its EC₅₀ concentration. In some embodiments the increase in ligand-induced TREM2-dependent gene transcription is at least 1-fold, at least 2-fold, at least a 3-fold, at least a 4-fold, at least a 5-fold, at least a 6-fold, at least a 7-fold, at least a 8-fold, at least a 9-fold, at least a 10-fold, at least an 11-fold, at least a 12-fold, at least a 13-fold, at least a 14-fold, at least a 15-fold, at least a 16-fold, at least a 17-fold, at least an 18-fold, at least a 19-fold, at least a 20-fold or greater when used at a concentration that ranges from about 0.5 nM to about 50 nM, or greater than 50 nM, and as compared to the level of TREM2-dependent gene transcription induced by binding of the TREM2 ligand to the TREM2 protein in the absence of the anti-TREM2 antibody when the TREM2 ligand is used at its EC₅₀ concentration. In some embodiments, the anti-TREM2 antibody is used at a concentration of at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at least 15 nM, at least 20 nM, at least 25 nM, at least 30 nM, at least 35 nM, at least 40 nM, at least 45 nM, at least 46 nM, at least 47 nM, at least 48 nM, at least 49 nM, or at least 50 nM. In some embodiments, the TREM2 ligand is phosphatidylserine (PS). In some embodiments, the TREM2 ligand is sphingomyelin (SM). In some embodiments, the increase in one more TEM2 activities may be measured by any suitable in vitro cell-based assays or suitable in vivo model described herein or known in the art. In some embodiments, a luciferase-based reporter assay is used to measure the fold increase of ligand-induced TREM2-dependent gene expression in the presence and absence of antibody, as described in Example 8, FIG. 10A-10F and FIG. 11A-11D.

As used herein, an anti-TREM2 antibody of the present disclosure does not compete with, inhibit, or otherwise block the interaction (e.g., binding) between one or more TREM2 ligands and TREM2 if it decreases ligand binding to TREM2 by less than 20% at saturating antibody concentrations utilizing any in vitro assay or cell-based culture assay described herein or known in the art. In some embodiments, anti-TREM2 antibodies of the present disclosure inhibit interaction (e.g., binding) between one or more TREM2 ligands and TREM2 by less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% at saturating antibody concentrations utilizing any in vitro assay or cell-based culture assay described herein or known in the art.

In some embodiments, an anti-TREM2 antibody of the present disclosure is an agonist antibody that induces one or more TREM2 activities. In some embodiments the antibody induces one or more activities of TREM2 after binding to a TREM2 protein that is expressed on a cell. In some embodiments the antibody induces one or more activities of TREM2 after binding to a soluble TREM2 protein that is not bound to the cell membrane. In certain embodiments the TREM2 protein is expressed on a cell surface. In certain embodiments, soluble TREM2 protein (sTREM2) may be found, without limitation, in extracellular milieu, in blood serum, in cerebrospinal fluid (CSF), and in the interstitial space within tissues. In certain embodiments, soluble TREM2 protein (sTREM2) is non-cellular. In some embodiments, anti-TREM2 antibodies of the present disclosure increase levels of soluble TREM2 protein (sTREM2) and/or increase the half-life of soluble TREM2 protein (sTREM2). In some embodiments a soluble TREM2 (sTREM2) protein of the present disclosure corresponds to amino acid residues 19-160 of SEQ ID NO:1. In some embodiments a soluble TREM2 (sTREM2) protein of the present disclosure corresponds to amino acid residues 19-159 of SEQ ID NO:1. In some embodiments a soluble TREM2 (sTREM2) protein of the present disclosure corresponds to amino acid residues 19-158 of SEQ ID NO:1. In some embodiments a soluble TREM2 (sTREM2) protein of the present disclosure corresponds to amino acid residues 19-157 of SEQ ID NO:1. In some embodiments a soluble TREM2 (sTREM2) protein of the present disclosure corresponds to amino acid residues 19-156 of SEQ ID NO:1. In some embodiments a soluble TREM2 (sTREM2) protein of the present disclosure corresponds to amino acid residues 19-155 of SEQ ID NO:1. In some embodiments a soluble TREM2 (sTREM2) protein of the present disclosure corresponds to amino acid residues 19-154 of SEQ ID NO:1.

In some embodiments, soluble TREM2 (sTREM2) proteins of the present disclosure may be inactive variants of cellular TREM2 receptors. In some embodiments, sTREM2 may be present in the periphery, such as in the plama, or brains of subject, and may sequester anti-TREM2 antibodies. Such sequestered antibodies would be unable to bind to and activate, for example, the cellular TREM2 resceptor present on cells. Accordingly, in certain embodiments, anti-TREM2 antibodies of the present disclosure, such as agonist anti-TREM2 antibodies of the present disclosure, do not bind to soluble TREM2. In some embodiments, anti-TREM2 antibodies of the present disclosure, such as agonist anti-TREM2 antibodies of the present disclosure, do not bind to soluble TREM2 in vivo. In some embodiments, agonist anti-TREM2 antibodies of the present disclosure that do not bind soluble TREM2 may bind to an epitope on TREM2 that, for example, may include a portion of the extracellular domain of cellular TREM2 that is not contained in sTREM2, for example one or more amino acid residues within amino acid residues 161-175; may be at or near a transmembrane portion of TREM2; or may include a transmembrane portion of TREM2. In some embodiments, such antibodies may bind to an epitope that includes amino acid residues E151, D152, H154, and E156 of SEQ ID NO: 1. In some embodiments, such antibodies may bind to an epitope that includes the N-terminal regions of the extra-cellular domain of TREM2. Thus, such anti-TREM2 antibodies bind cellular TREM2 without binding soluble TREM2. Advantageously, such anti-TREM2 antibodies will not be questered by sTREM2 present, for example, in the periphery or brain, and will thus be available to activate the cellular TREM2 receptor present on cells.

The TREM2 activities induced by anti-TREM2 antibodies of the present disclosure may include, (a) modulated expression of one or more anti-inflammatory cytokines, optionally wherein the one or more anti-inflammatory cytokines are selected from IL-4, IL-10 TGF-β, IL-13, IL-35 IL-16, IFN-alpha, IL-1Ra, VEGF, G-CSF, YM, AXL, FLT1 and soluble receptors for TNF or IL-6; (b) modulated expression of one or more anti-inflammatory cytokines in one or more cells selected from macrophages, dendritic cells, bone marrow-derived dendritic cells, monocytes, osteoclasts, and microglial cells; (c) modulated expression of one or more pro-inflammatory cytokines, optionally wherein the one or more pro-inflammatory cytokines are selected from IFN-β, IL-1α, IL-1β, TNF-α, IL-6, IL-8, CRP, IL-20 family members, IL-33, LIF, IFN-gamma, OSM, CNTF, GM-CSF, IL-11, IL-12, IL-17, IL-18, IL-23, CXCL10, CCL4, and MCP-1; (d) modulated expression of one or more pro-inflammatory cytokines in one or more cells selected from macrophages, dendritic cells, bone marrow-derived dendritic cells, monocytes, osteoclasts, and microglial cells; (e) activation of extracellular signal-regulated kinase (ERK) phosphorylation; (f) activating tyrosine phosphorylation on multiple cellular proteins; (g) modulated expression of C-C chemokine receptor 7 (CCR7); (h) activation of microglial cell chemotaxis toward CCL19 and CCL21 expressing cells; (i) increasing priming and/or modulating function of one or more T cells, such as CD8+ T cells, CD4+ T cells and/or regulatory T cell by one or more cells selected from dendritic cells, bone marrow-derived dendritic cells, monocytes, microglia, M1 microglia, activated M1 microglia, M2 microglia, macrophages, M1 macrophages, activated M1 macrophages, and M2 macrophages; (j) activation of osteoclast production, increased rate of osteoclastogenesis, or both; (k) increased survival of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (1) increased proliferation of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (m) activating migration of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (n) activating one or more functions of one or more cells selected from t dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (o) activating maturation of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (p) activating of one or more types of clearance selected from apoptotic neuron clearance, nerve tissue debris clearance, non-nerve tissue debris clearance, bacteria clearance, other foreign body clearance, disease-causing protein clearance, disease-causing peptide clearance, and tumor cell clearance; optionally wherein the disease-causing protein is selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides and the tumor cell is from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer; (q) inhibition of phagocytosis of one or more of apoptotic neurons, nerve tissue debris, non-nerve tissue debris, bacteria, other foreign bodies, disease-causing proteins, disease-causing peptides, disease-causing nucleic acids, or tumor cells; optionally wherein the disease-causing nucleic acids are antisense GGCCCC (G2C4) repeat-expansion RNA, the disease-causing proteins are selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the tumor cells are from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, or thyroid cancer; (r) binding to TREM2 ligand on tumor cells; (s) binding to TREM2 ligand on cells selected from neutrophils, dendritic cells, bone marrow-derived dendritic cells, monocytes, microglia, and macrophages; (t) activation of tumor cell killing by one or more of microglia, macrophages, dendritic cells, bone marrow-derived dendritic cells, neutrophils, T cells, T helper cells, or cytotoxic T cells; (u) activating anti-tumor cell proliferation activity of one or more of microglia, macrophages, dendritic cells, bone marrow-derived dendritic cells, neutrophils, T cells, T helper cells, or cytotoxic T cells; (v) activating anti-tumor cell metastasis activity of one or more of microglia, macrophages, dendritic cells, bone marrow-derived dendritic cells, neutrophils, T cells, T helper cells, or cytotoxic T cells; (w) activating of one or more ITAM motif containing receptors, optionally wherein the one or more ITAM motif containing receptors are selected from TREM1, TREM2, FcgR, DAP10, and DAP12; (x) activating of signaling by one or more pattern recognition receptors (PRRs), optionally wherein the one or more PRRs are selected from receptors that identify pathogen-associated molecular patterns (PAMPs), receptors that identify damage-associated molecular patterns (DAMPs), and any combination thereof; (y) activating of one or more receptors comprising the motif D/Ex₀₋₂YxxL/IX₆₋₈YxxL/I (SEQ ID NO: 883); (z) activating of signaling by one or more Toll-like receptors; (aa) activating of the JAK-STAT signaling pathway; (bb) activating of nuclear factor kappa-light-chain-enhancer of activated B cells (NFκB); (cc) phosphorylation of an ITAM motif containing receptor; (dd) modulated expression of one or more inflammatory receptors, optionally wherein the one or more inflammatory receptors comprise CD86 and the one or more inflammatory receptors are expressed on one or more of microglia, macrophages, dendritic cells, bone marrow-derived dendritic cells, neutrophils, T cells, T helper cells, or cytotoxic T cells; (ee) increasing expression of one or more TREM2-dependent genes; (gg) normalization of disrupted TREM2-dependent gene expression; (ff) increasing expression of one or more ITAM-dependent genes, optionally wherein the one more ITAM-dependent genes are activated by nuclear factor of activated T cells (NFAT) transcription factors; (gg) inhibiting differentiation of one or more of immunosuppressor dendritic cells, immunosuppressor macrophages, myeloid derived suppressor cells, tumor-associated macrophages, immunosuppressor neutrophils, and regulatory T cells; (hh) inhibiting functionality of one or more of immunosuppressor dendritic cells, immunosuppressor macrophages, myeloid-derived suppressor cells, tumor-associated macrophages, immunosuppressor neutrophils, and regulatory T cells; (ii) decreasing infiltration of one or more of immunosuppressor dendritic cells, immunosuppressor macrophages, myeloid derived suppressor cells, tumor-associated macrophages, immunosuppressor neutrophils, and regulatory T cells into tumors; (jj) decreasing number of tumor-promoting myeloid/granulocytic immune-suppressive cells in a tumor, in peripheral blood, or other lymphoid organ; (kk) inhibiting tumor-promoting activity of myeloid-derived suppressor cells; (11) decreasing expression of tumor-promoting cytokines in a tumor or in peripheral blood, optionally wherein the tumor-promoting cytokines are TGF-beta or IL-10; (mm) decreasing tumor infiltration of tumor-promoting FoxP3+ regulatory T lymphocytes; (nn) increasing activation of tumor-specific T lymphocytes with tumor killing potential; (oo) decreasing tumor volume; (pp) decreasing tumor growth rate; (qq) increasing efficacy of one or more immune-therapies that modulate anti-tumor T cell responses, optionally wherein the one or more immune-therapies are selected from f PD1/PDL1 blockade, CTLA-4 blockade, and cancer vaccines; (rr) inhibition of PLCγ/PKC/calcium mobilization; and (uu) inhibition of PI3K/Akt, Ras/MAPK signaling. (ss) increasing phagocytosis by dendritic cells, macrophages, monocytes, and/or microglia (tt) induction or retention of TREM2 clustering on a cell surface; (xx) TREM2 binding to DAP12; (uu) TREM2 phosphorylation; (vv) DAP12 phosphorylation; (ww) TREM2 phosphorylation; (xx) activation of one or more SRC family tyrosine kinases including Syk kinase; (yy) increasing memory; and (zz) reducing cognitive deficit.

Anti-TREM2 antibodies of the present disclosure can be used to prevent, reduce risk of, or treat dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, solid and blood cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza. The methods provided herein also find use in inducing or promoting the survival, maturation, functionality, migration, or proliferation of one or more immune cells in an individual in need thereof. The methods provided herein find further use in decreasing the activity, functionality, or survival of regulatory T cells, tumor-imbedded immunosuppressor dendritic cells, tumor-imbedded immunosuppressor macrophages, myeloid-derived suppressor cells, tumor-associated macrophages, acute myeloid leukemia (AML) cells, chronic lymphocytic leukemia (CLL) cell, or chronic myeloid leukemia (CML) cell in an individual in need thereof. The methods provided herein find further use in increasing memory and/or reducing cognitive deficit.

The anti-TREM2 antibodies of the present disclosure may also be used in advanced wound care. In some embodiments, the anti-TREM2 antibodies of the present disclosure are monoclonal antibodies. Anti-TREM2 antibodies of the present disclosure may be tested for inducing one or more TREM2 activities (a) modulated expression of one or more anti-inflammatory cytokines, optionally wherein the one or more anti-inflammatory cytokines are selected from IL-4, IL-10 TGF-β, IL-13, IL-35 IL-16, IFN-alpha, IL-1Ra, VEGF, G-CSF, YM, AXL, FLT1 and soluble receptors for TNF or IL-6; (b) modulated expression of one or more anti-inflammatory cytokines in one or more cells selected from macrophages, dendritic cells, bone marrow-derived dendritic cells, monocytes, osteoclasts, and microglial cells; (c) modulated expression of one or more pro-inflammatory cytokines, optionally wherein the one or more pro-inflammatory cytokines are selected from IFN-β, IL-1α, IL-1β, TNF-α, YM-1, CD86, CCL2, CCL3, CCL5, CCR2, Gata3, Rorc, IL-6, IL-8, CRP, IL-20 family members, IL-33, LIF, IFN-gamma, OSM, CNTF, GM-CSF, IL-11, IL-12, IL-17, IL-18, IL-23, CXCL10, CCL4, FLT1, CSF-1, OPN, MHC-II, CD11c, AXL and MCP-1; (d) modulated expression of one or more pro-inflammatory cytokines in one or more cells selected from macrophages, dendritic cells, bone marrow-derived dendritic cells, monocytes, osteoclasts, and microglial cells; (e) activation of extracellular signal-regulated kinase (ERK) phosphorylation; (f) activating tyrosine phosphorylation on multiple cellular proteins; (g) modulated expression of C-C chemokine receptor 7 (CCR7); (h) activation of microglial cell chemotaxis toward CCL19 and CCL21 expressing cells; (i) increasing priming or modulating function of T cells, such as CD8+ T cells, CD4+ T cells, and/or regulatory T cells, induced by one or more cells selected from dendritic cells, bone marrow-derived dendritic cells, monocytes, microglia, M1 microglia, activated M1 microglia, M2 microglia, macrophages, M1 macrophages, activated M1 macrophages, and M2 macrophages; (j) activation of osteoclast production, increased rate of rate of osteoclastogenesis, or both; (k) increased survival of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (1) increased proliferation of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (m) activating migration of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (n) activating one or more functions of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (o) activating maturation of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (p) activating of one or more types of clearance selected from apoptotic neuron clearance, nerve tissue debris clearance, non-nerve tissue debris clearance, bacteria clearance, other foreign body clearance, disease-causing protein clearance, disease-causing peptide clearance, and tumor cell clearance; optionally wherein the disease-causing protein is selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides and the tumor cell is from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer; (u) activation of phagocytosis of one or more of apoptotic neurons, nerve tissue debris, non-nerve tissue debris, bacteria, other foreign bodies, disease-causing proteins, disease-causing peptides, disease-causing nucleic acids, or tumor cells; optionally wherein the disease-causing nucleic acids are antisense GGCCCC (G2C4) repeat-expansion RNA, the disease-causing proteins are selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the tumor cells are from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, or thyroid cancer; (p) binding to TREM2 ligand on tumor cells; (q) binding to TREM2 ligand on cells selected from neutrophils, dendritic cells, bone marrow-derived dendritic cells, monocytes, microglia, and macrophages; (r) activation of tumor cell killing by one or more of microglia, macrophages, dendritic cells, bone marrow-derived dendritic cells, neutrophils, T cells, T helper cells, or cytotoxic T cells; (s) activating anti-tumor cell proliferation activity of one or more of microglia, macrophages, dendritic cells, bone marrow-derived dendritic cells, neutrophils, T cells, T helper cells, or cytotoxic T cells; (t) activating anti-tumor cell metastasis activity of one or more of microglia, macrophages, dendritic cells, bone marrow-derived dendritic cells, neutrophils, T cells, T helper cells, or cytotoxic T cells; (y) activating of one or more ITAM motif containing receptors, optionally wherein the one or more ITAM motif containing receptors are selected from TREM1, TREM2, FcgR, DAP10, and DAP12; (z) activating of signaling by one or more pattern recognition receptors (PRRs), optionally wherein the one or more PRRs are selected from receptors that identify pathogen-associated molecular patterns (PAMPs), receptors that identify damage-associated molecular patterns (DAMPs), and any combination thereof; (aa) activating of one or more receptors comprising the motif D/Ex₀₋₂YxxL/IX₆₋₈YxxL/I (SEQ ID NO: 883); (bb) activating of signaling by one or more Toll-like receptors; (cc) activating of the JAK-STAT signaling pathway; (dd) activating of nuclear factor kappa-light-chain-enhancer of activated B cells (NFκB); (dd) phosphorylation of an ITAM motif containing receptor; (ee) modulated expression of one or more inflammatory receptors, optionally wherein the one or more inflammatory receptors comprise CD86 and the one or more inflammatory receptors are expressed on one or more of microglia, macrophages, dendritic cells, bone marrow-derived dendritic cells, neutrophils, T cells, T helper cells, or cytotoxic T cells; (ff) increasing expression of one or more TREM2-dependent genes; (gg) normalization of disrupted TREM2-dependent gene expression; (hh) increasing expression of one or more ITAM-dependent genes, optionally wherein the one more ITAM-dependent genes are activated by nuclear factor of activated T cells (NFAT) transcription factors; (ii) inhibiting differentiation of one or more of immunosuppressor dendritic cells, immunosuppressor macrophages, myeloid derived suppressor cells, tumor-associated macrophages, immunosuppressor neutrophils, and regulatory T cells; (jj) inhibiting functionality of one or more of immunosuppressor dendritic cells, immunosuppressor macrophages, myeloid-derived suppressor cells, tumor-associated macrophages, immunosuppressor neutrophils, and regulatory T cells; (nn) decreasing infiltration of one or more of immunosuppressor dendritic cells, immunosuppressor macrophages, myeloid derived suppressor cells, tumor-associated macrophages, immunosuppressor neutrophils, and regulatory T cells into tumors; (kk) decreasing number of tumor-promoting myeloid/granulocytic immune-suppressive cells in a tumor, in peripheral blood, or other lymphoid organ; (11) inhibiting tumor-promoting activity of myeloid-derived suppressor cells; (mm) decreasing expression of tumor-promoting cytokines in a tumor or in peripheral blood, optionally wherein the tumor-promoting cytokines are TGF-beta or IL-10; (nn) decreasing tumor infiltration of tumor-promoting FoxP3+ regulatory T lymphocytes; (oo) increasing activation of tumor-specific T lymphocytes with tumor killing potential; (pp) decreasing tumor volume; (qq) decreasing tumor growth rate; (rr) increasing efficacy of one or more immune-therapies that modulate anti-tumor T cell responses, optionally wherein the one or more immune-therapies are selected from PD1/PDL1 blockade, CTLA-4 blockade, and cancer vaccines; (ww) inhibition of PLCy/PKC/calcium mobilization; and (xx) inhibition of PI3K/Akt, Ras/MAPK signaling. (xx) increasing phagocytosis by dendritic cells, macrophages, monocytes, and/or microglia (yy) induction or retention of TREM2 clustering on a cell surface; (zz) TREM2 binding to DAP12; (aaa) TREM2 phosphorylation; (bbb) DAP12 phosphorylation; (ccc) TREM2 autophosphorylation; (ddd) activation of one or more SRC family tyrosine kinases including Syk kinase; (eee) modulating expression of one or more proteins selected from the group consisting of C1qa, C1qB, C1qC, C1s, C1R, C4, C2, C3, ITGB2, HMOX1, LAT2. CASP1, CSTA, VSIG4, MS4A4A, C3AR1, GPX1, TyroBP, ALOXSAP, ITGAM, SLC7A7, CD4, ITGAX, PYCARD, and VEGF; (fff) increasing memory; and (ggg) reducing cognitive deficit. Useful assays may include western blots (e.g., for tyrosine-phosphorylated DAP12 or threonine/serine-phosphorylated PI3K-kinase substrates), ELISA (e.g., for secreted interleukin or cytokine secretion), FACS (e.g., for anti-TREM2 binding to TREM2), immunocytochemistry (e.g., for e.g., for tyrosine-phosphorylated DAP12 or threonine/serine-phosphorylated PI3K-kinase substrates), reporter-gene assays (e.g., for TLR activation), increased survival and/or function of dendritic cells, macrophages, monocytes, osteoclasts, Langerhans cells of skin, Kupffer cells, and/or microglia, increased phagocytosis of apoptotic neurons, damaged synapses, amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, and proline-arginine (PR) repeat peptides, nerve tissue debris, non-nerve tissue debris, bacteria, other foreign bodies, disease-causing proteins, disease-causing peptides, disease-causing nucleic acid, or tumor cells by macrophages, dendritic cells, Langerhans cells of skin, Kupffer cells, monocytes, osteoclasts, and/or microglial cells, increased cytoskeleton reorganization, and decreased microglial pro-inflammatory responses, or other assays known in the art.

An antibody dependent on binding to FcgR receptor to activate targeted receptors may lose its agonist activity if engineered to eliminate FcgR binding (see, e.g., Wilson et al., (2011) Cancer Cell 19, 101-113; Armour at al., (2003) Immunology 40 (2003) 585-593); and White et al., (2015) Cancer Cell 27, 138-148). As such, it is thought that an anti-TREM2 antibody of the present disclosure with the correct epitope specificity can be an agonist antibody and activate the target antigen, with minimal adverse effects, when the antibody has an Fc domain from a human IgG2 isotype (CH1 and hinge region) or another type of Fc domain that is capable of preferentially binding the inhibitory FcgRIIB r receptors, or a variation thereof.

Exemplary agonist antibody Fc isotypes and modifications are provided in Table A below. In some embodiments, the agonist antibody has an Fc isotype listed in Table A below.

TABLE A
Exemplary anti-TREM2 antibody Fc isotypes that are
capable of binding Fc gamma receptor

| Fc Isotype | Mutation (EU numbering scheme) |
|---|---|
| IgG1 | N297A |
| IgG1 | D265A and N297A |
| IgG1 | D270A |
| IgG1 | L234A and L235A |
| | L234A and G237A |
| | L234A and L235A and G237A |
| IgG1 | P238D and/or L328E and/or S267E/L328F and/or E233 and |
| | or/G237D and/or H268D and/or P271G and/or A330R |
| IgG1 | P238D and L328E and E233D and G237D and H268D and |
| | P271G and A330R |
| IgG1 | P238D and L328E and G237D and H268D and P271G and |
| | A330R |
| IgG1 | P238D and S267E and L328F and E233D and G237D and |
| | H268D and P271G and A330R |
| IgG1 | P238D and S267E and L328F and G237D and H268D and |
| | P271G and A330R |
| IgG2 | V234A and G237A |
| IgG4 | L235A and G237A and E318A |
| IgG4 | S228P and L236E |
| IgG2/4 | IgG2 aa 118 to 260 and IgG4 aa 261 to 447 |
| hybrid | H268Q and V309L; and A330S and P331S |
| IgG1 | C226S and C229S and E233P and L234V and L235A |
| IgG1 | L234F and L235E and P331S |
| IgG2 | C232S or C233S |
| IgG2 | A330S and P331S |
| IgG1 | S267E, and L328F |
| | S267E alone |
| IgG2 | S267E and L328F |
| IgG4 | S267E and L328F |
| IgG2 | WT HC with Kappa (light chain) LC |
| | HC C127S with Kappa LC |
| | Kappa LC C214S |
| | Kappa LC C214S and HC C233S |
| | Kappa LC C214S and HC C232S |
| | Any of the above listed mutations together with P330S and |
| | P331S mutations |
| | F(ab′)2 fragment of WT IgG1 and any of the above listed |
| | mutations |
| IgG1 | Substitute the Constant Heavy 1 (CH1) and hinge region of |
| | IgG1 With CH1 and hinge region of IGg2 |
| | ASTKGPSVFP LAPCSRSTSE STAALGCLVK |
| | DYFPEPVTVS WNSGALTSGV HTFPAVLQSS |
| | GLYSLSSVVT VPSSNFGTQT YTCNVDHKPS |
| | NTKVDKTVER KCCVECPPCP (SEQ ID NO: 886) |
| | With a Kappa LC |
| IgG1 | Any of the above listed mutations together with A330L and/ |
| | or L234F and/or L235E and/or P331S |
| IgG1, IgG2, | Any of the above listed mutations together with M252Y |
| or IgG4 | and/or S254T and/or T256E |
| Mouse IgG1 | For mouse disease models |
| IgG4 | WT |

In addition to the isotypes described in Table A, and without wishing to be bound to theory, it is thought that antibodies with human IgG1 or IgG3 isotypes and mutants thereof (e.g. Strohl (2009) Current Opinion in Biotechnology 2009, 20:685-691) that bind the activating Fcg Receptors I, HA, IIC, MA, BIB in human and/or Fcg Receptors I, III and IV in mouse, may also act as agonist antibodies in vivo but may be associated with adverse effects related to ADCC. However, such Fcg receptors appear to be less available for antibody binding in vivo, as compared to the Inhibitory Fcg receptor FcgRIIB (see, e.g., White, et al., (2013) Cancer Immunol. Immunother. 62, 941-948; and Li et al., (2011) Science 333(6045):1030-1034.).

In some embodiments, the agonist antibody is of the IgG class, the IgM class, or the IgA class. In some embodiments, the agonist antibody has an IgG1, IgG2, IgG3, or IgG4 isotype.

In certain embodiments, the agonist antibody has an IgG2 isotype. In some embodiments, the agonist antibody contains a human IgG2 constant region. In some embodiments, the human IgG2 constant region includes an Fc region. In some embodiments, the agonist antibody induces the one or more TREM2 activities, the DAP12 activities, or both independently of binding to an Fc receptor. In some embodiments, the agonist antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from V234A (Alegre et al., (1994) Transplantation 57:1537-1543. 31; Xu et al., (2000) Cell Immunol, 200:16-26), G237A (Cole et al. (1999) Transplantation, 68:563-571), H268Q, V309L, A330S, P331S (US 2007/0148167; Armour et al. (1999) Eur J Immunol 29: 2613-2624; Armour et al. (2000) The Haematology Journal 1(Supp1.1):27; Armour et al. (2000) The Haematology Journal 1(Supp1.1):27), C232S, and/or C233S (White et al. (2015) Cancer Cell 27, 138-148), S267E, L328F (Chu et al., (2008) Mol Immunol, 45:3926-3933), M252Y, S254T, and/or T256E, where the amino acid position is according to the EU or, Kabat numbering convention.

In some embodiments, the agonist antibody has an IgG2 isotype with a heavy chain constant domain that contains a C127S amino acid substitution, where the amino acid position is according to the EU or, Kabat numbering convention (White et al., (2015) Cancer Cell 27, 138-148; Lightle et al., (2010) PROTEIN SCIENCE 19:753-762; and WO2008079246).

In some embodiments, the agonist antibody has an IgG2 isotype with a Kappa light chain constant domain that contains a C214S amino acid substitution, where the amino acid position is according to the EU or, Kabat numbering convention (White et al., (2015) Cancer Cell 27, 138-148; Lightle et al., (2010) PROTEIN SCIENCE 19:753-762; and WO2008079246).

In certain embodiments, the agonist antibody has an IgG1 isotype. In some embodiments, the agonist antibody contains a mouse IgG1 constant region. In some embodiments, the agonist antibody contains a human IgG1 constant region. In some embodiments, the human IgG1 constant region includes an Fc region. In some embodiments, the agonist antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU or, Kabat numbering convention.

In some embodiments, the antibody includes an IgG2 isotype heavy chain constant domain 1(CH1) and hinge region (White et al., (2015) Cancer Cell 27, 138-148). In certain embodiments, the IgG2 isotype CH1 and hinge region contain the amino acid sequence of ASTKGPSVFPLAPCSRSTSESTAALGCLVKDYFPEPVTVSWNSGALTSGVHTFPAVLQSSGLY SLSSVVTVPSSNFGTQTYTCNVDHKPSNTKVDKTVERKCCVECPPCP (SEQ ID NO: 886). In some embodiments, the antibody Fc region contains a S267E amino acid substitution, a L328F amino acid substitution, or both, and/or a N297A or N297Q amino acid substitution, where the amino acid position is according to the EU or, Kabat numbering convention.

In certain embodiments, the agonist antibody has an IgG4 isotype. In some embodiments, the agonist antibody contains a human IgG4 constant region. In some embodiments, the human IgG4 constant region includes an Fc region. In some embodiments, the agonist antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from L235A, G237A, S228P, L236E (Reddy et al., (2000) J Immunol, 164:1925-1933), S267E, E318A, L328F, M252Y, S254T, and/or T256E, where the amino acid position is according to the EU or, Kabat numbering convention.

In certain embodiments, the agonist antibody has a hybrid IgG2/4 isotype. In some embodiments, the agonist antibody includes an amino acid sequence containing amino acids 118 to 260 according to EU or, Kabat numbering of human IgG2 and amino acids 261-447 according to EU or, Kabat numbering of human IgG4 (WO 1997/11971; WO 2007/106585).

In certain embodiments, the antibody contains a mouse IgG4 constant region (Bartholomaeus, et al. (2014). J. Immunol. 192, 2091-2098).

In some embodiments, the Fc region further contains one or more additional amino acid substitutions selected from A330L, L234F; L235E, or P331S according to EU or, Kabat numbering; and any combination thereof.

Inert Antibodies

Another class of antibodies of the present disclosure includes inert antibodies. As used herein, “inert” antibodies refer to antibodies that specifically bind their target antigen but do not modulate (e.g., decrease/inhibit or activate/induce) antigen function. For example, in the case of TREM2, inert antibodies do not modulate ligand binding and/or TREM2 activities. Without wishing to be bound to theory, it is thought that antibodies that do not have the ability to cluster TREM2 on the cell surface may be inert antibodies even if they have an epitope specificity that is compatible with receptor activation.

In some embodiments, antibodies that bind a TREM2 protein may include antibodies that bind TREM2 but, due to their epitope specificity, do not modulate protein function. Such functionally inert antibodies can be used as cargo to transport toxins or to tumor cells as described for the CD33 antibody Gemtuzumab zogamicin, (marketed as Mylotarg) which is conjugated to the cytotoxic agent from the class of calicheamicins and is used to target and kill acute myelogenous leukemia tumors (Naito et al., (2000), Leukemia, 14, 1436-1443; Ricart (2011) Clin Cancer Res 17; 6417-6436; Hamann et al., (2002) Journal: Bioconjugate Chemistry, 13, 47-58; and Beitz et al., (2001) Clin Cancer Res 7; 1490-6.). Therefore, in some embodiments, antibodies of the present disclosure are inert antibodies that bind TREM2 but are incapable of inducing one or more TREM2 activities (e.g., a TREM2 activity described herein).

Exemplary inert antibody Fc isotypes and modifications are provided in Table B below. In some embodiments, the inert antibody has an Fc isotype listed in Table B below.

Antagonist Antibodies

A third class of antibodies of the present disclosure includes antagonist antibodies. In some embodiments, antibodies that bind a TREM2 protein may include antagonist antibodies that bind TREM2 and inhibit one or more TREM2 activities, either by preventing interaction between TREM2 and one or more TREM2 ligands, or by preventing the transduction of signal from the extracellular domain of TREM2 into the cell cytoplasm in the presence of ligand. In some embodiments, antagonist antibodies of the present disclosure may have the epitope specificity of an agonist antibody of the present disclosure, but have an Fc domain that is not capable of binding Fcg receptors and thus is unable to, for example, cluster the TREM2 receptor.

In some embodiments, an antibody of the present disclosure is an antagonist antibody. In some embodiments, the antagonist antibody inhibits one or more TREM2 activities. In some embodiments, the antagonist antibody decreases activity of one or more TREM2-dependent genes. In some embodiments, the anti-TREM2 antibody decreases levels of TREM2 in one or more cells (e.g., cell surface levels, intracellular levels, or total levels). In some embodiments, the anti-TREM2 antibody induces degradation of TREM2. In some embodiments, the anti-TREM2 antibody induces cleavage of TREM2. In some embodiments, the anti-TREM2 antibody induces internalization of TREM2. In some embodiments, the anti-TREM2 antibody induces shedding of TREM2. In some embodiments, the anti-TREM2 antibody induces downregulation of TREM2 expression. In some embodiments, the anti-TREM2 antibody inhibits interaction (e.g., binding) between TREM2 and one or more TREM2 ligands. In some embodiments, the anti-TREM2 antibody transiently activates and then induces degradation of TREM2. In some embodiments, the anti-TREM2 antibody transiently activates and then induces cleavage of TREM2. In some embodiments, the anti-TREM2 antibody transiently activates and then induces internalization of TREM2. In some embodiments, the anti-TREM2 antibody transiently activates and then induces shedding of TREM2. In some embodiments, the anti-TREM2 antibody transiently activates and then induces downregulation of TREM2 expression. In some embodiments, the anti-TREM2 antibody transiently activates and then induces decreased expression of TREM2. In certain embodiments, the individual has a TREM2 variant allele. In some embodiments, the anti-TREM2 antibody acts in solution.

In some embodiments, the one or more TREM2-dependent genes include, without limitation, one or more nuclear factor of activated T-cells (NFAT) transcription factors. In some embodiments, the antagonist antibody decreases the survival of macrophages, microglial cells, M1 macrophages, M1 microglial cells, M2 macrophages, M2 microglial cells, osteoclasts, Langerhans cells of skin, Kupffer cells, and/or dendritic cells. In some embodiments, the antagonist antibody inhibits interaction between TREM2 and one or more TREM2 ligands. In some embodiments, the antagonist antibody inhibits TREM2 signal transduction. In some embodiments, the antagonist antibody inhibits interaction between TREM2 and one or more TREM2 ligands and inhibits TREM2 signal transduction. In some embodiments, the antagonist antibody inhibits TREM2 interaction with DAP12.

Levels of TREM2 in one or more cells (e.g., cellular levels) may refer to, without limitation, cell surface levels of TREM2, intracellular levels of TREM2, and total levels of TREM2. In some embodiments, a decrease in cellular levels of TREM2 comprises decrease in cell surface levels of TREM2. As used herein, cell surface levels of TREM2 may be measured by any in vitro cell-based assays or suitable in vivo model described herein or known in the art, for example, utilizing flow cytometry, such as fluorescence-activated cell sorting (FACS), to measure cell surface levels of TREM2. In some embodiments, a decrease in levels of TREM2 in cells comprises a decrease in intracellular levels of TREM2. As used herein intracellular levels of TREM2 may be measured by any in vitro cell-based assays or suitable in vivo model described herein or known in the art, for example immunostaining, Western blot analysis, co-immunoprecipitation, and cell cytometry. In some embodiments, a decrease in cellular levels of TREM2 comprises a decrease in total levels of TREM2. As used herein, total levels of TREM2 may be measured by any in vitro cell-based assays or suitable in vivo model described herein or known in the art, for example immunostaining, Western blot analysis, co-immunoprecipitation, and cell cytometry. In some embodiments, the anti-TREM2 antibodies induce TREM2 degradation, TREM2 cleavage, TREM2 internalization, TREM2 shedding, and/or downregulation of TREM2 expression. In some embodiments, levels of TREM2 in one or more cells (e.g., cellular levels) are measured on primary cells (e.g., dendritic cells, bone marrow-derived dendritic cells, monocytes, microglia, and macrophages) or on cell lines utilizing an in vitro cell assay. In some embodiments, anti-TREM2 antibodies of the present disclosure decrease cellular levels of TREM2 by at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more as compared to cellular levels of TREM2 in the absence of the anti-TREM2 antibody. Any in vitro cell-based assays or suitable in vivo model described herein or known in the art may be used to measure inhibition of interaction (e.g., binding) between TREM2 and one or more TREM2 ligands. In some embodiments, anti-TREM2 antibodies of the present disclosure inhibit interaction (e.g., binding) between TREM2 and one or more TREM2 ligands by a at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more at saturating antibody concentrations utilizing any in vitro assay or cell-based culture assay described herein or known in the art.

In some embodiments, antibody cross-linking is required for agonist antibody function. Antibody cross-linking can occur through binding to a secondary antibody in vitro or through binding to Fc receptors in vivo. For example, antagonistic antibodies can be converted to agonistic antibodies via biotin/streptavidin cross-linking or secondary antibody binding in vitro (see for example Gravestein et al., (1996) J. Exp. Med. 184:675-685; Gravestein et al., (1994) International Immunol. 7:551-557). Agonistic antibodies may exert their activity by mimicking the biological activity of the receptor ligand or by enhancing receptor aggregation, thereby activating receptor signaling. In some embodiments, the absence of antibody cross-linking is required for antagonistic activity. In some embodiments, the antibody will act as antagonistic when presented as monomer and as an agonist when presented as a dimer or a multimer. Antagonistic antibodies may exert their activity by blocking receptor-ligand interactions.

Exemplary antagonist antibody Fc isotypes and modifications are provided in Table B below. In some embodiments, the antagonist antibody has an Fc isotype listed in Table B below.

Exemplary Fc Isotypes of Inert and Antagonist Antibodies

In some embodiments, inert and/or antagonist anti-TREM antibodies have an Fc isotype listed in Table B below.

TABLE B
Exemplary anti-TREM2 antibody Fc isotypes with reduced
binding to Fc gamma receptor

| Fc Isotype | Mutation (EU numbering scheme) |
|---|---|
| IgG1 | N297A or N297Q |
| IgG1 | D265A and N297A |
| IgG1 | L234A and L235A |
| IgG2 | V234A and G237A |
| IgG4 | F235A and G237A and E318A |
| | E233P and/or F234V |
| | N297A or N297Q |
| IgG4 | S228P and L236E |
| | S241P |
| | S241P and L248E |
| | S228P and F234A and L235A |
| IgG2 | H268Q and V309L and A330S and P331S |
| IgG1 | C220S and C226S and C229S and P238S |
| IgG1 | C226S and C229S and E233P and L234V, and L235A |
| IgG1 | E233P and L234V and L235A and G236-deleted |
| | P238A |
| | D265A |
| | N297A |
| | A327Q or A327G |
| | P329A |
| IgG1 | K322A and L234A and L235A |
| IgG1 | L234F and L235E and P331S |
| IgG1 or IgG4 | T394D |
| IgG2 | C232S or C233S |
| | N297A or N297Q |
| IgG2 | V234A and G237A and P238S and H268A and V309L |
| | and A330S and P331S |
| IgG1, IgG2, | delta a, b, c, ab, ac, g modifications |
| or IgG4 | |
| IgG1 | Any of the above listed mutations together with A330L |
| | or L234F and/or L235E and/or P331S |
| IgG1, IgG2, | Any of the above listed mutations together with |
| or IgG4 | M252Y and/or S254T and/or T256E |

In certain embodiments, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from N297A, N297Q (Bolt S et al. (1993) Eur J Immunol 23:403-411), D265A, L234A, L235A (McEarchern et al., (2007) Blood, 109:1185-1192), C226S, C229S (McEarchern et al., (2007) Blood, 109:1185-1192), P238S (Davis et al., (2007) J Rheumatol, 34:2204-2210), E233P, L234V (McEarchern et al., (2007) Blood, 109:1185-1192), P238A, A327Q, A327G, P329A (Shields R L. et al., (2001) J Biol Chem. 276(9):6591-604), K322A, L234F, L235E (Hezareh, et al., (2001) J Virol 75, 12161-12168; Oganesyan et al., (2008). Acta Crystallographica 64, 700-704), P331S (Oganesyan et al., (2008) Acta Crystallographica 64, 700-704), T394D (Wilkinson et al. (2013) MAbs 5(3): 406-417), A330L, M252Y, S254T, and/or T256E, where the amino acid position is according to the EU or, Kabat numbering convention. In certain embodiments, the Fc region further includes an amino acid deletion at a position corresponding to glycine 236 according to the EU or, Kabat numbering convention.

In some embodiments, the antibody has an IgG1 isotype with a heavy chain constant region that contains a C220S amino acid substitution according to the EU or, Kabat numbering convention.

In some embodiments, the Fc region further contains one or more additional amino acid substitutions selected from t A330L, L234F; L235E, and/or P331S according to EU or, Kabat numbering convention.

In certain embodiments, the antibody has an IgG2 isotype. hi some embodiments, the antibody contains a human IgG2 constant region. In some embodiments, the human IgG2 constant region includes an Fc region. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from V234A, G237A, H268E, V309L, N297A, N297Q, A330S, P331S, C232S, C233S, M252Y, S254T, and/or T256E, where the amino acid position is according to the EU or, Kabat numbering convention.

In certain embodiments, the antibody has an IgG4 isotype. In some embodiments, the antibody contains a human IgG4 constant region. In some embodiments, the human IgG4 constant region includes an Fc region. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from E233P, F234V, L235A, G237A, E318A (Hutchins et al. (1995) Proc Natl Acad Sci USA, 92:11980-11984), S228P, L236E, S241P, L248E (Reddy et al., (2000) J Immunol, 164:1925-1933; Angal et al., (1993) Mol Immunol. 30(1):105-8; U.S. Pat. No. 8,614,299 B2), T394D, M252Y, S254T, T256E, and/or N297A, N297Q, where the amino acid position is according to the EU or, Kabat numbering convention.

In some embodiments, the Fc region further contains one or more additional amino acid substitutions selected from a M252Y, S254T, and/or T256E, where the amino acid position is according to the EU or, Kabat numbering convention.

Further IgG Mutations

In some embodiments, one or more of the IgG1 variants described herein may be combined with an A330L mutation (Lazar et al., (2006) Proc Natl Acad Sci USA, 103:4005-4010), or one or more of L234F, L235E, and/or P331S mutations (Sazinsky et al., (2008) Proc Natl Acad Sci USA, 105:20167-20172), where the amino acid position is according to the EU or, Kabat numbering convention, to eliminate complement activation. In some embodiments, the IgG variants described herein may be combined with one or more mutations to enhance the antibody half-life in human serum (e.g. M252Y, S254T, T256E mutations according to the EU or, Kabat numbering convention) (Dall'Acqua et al., (2006) J Biol Chem, 281:23514-23524; and Strohl e al., (2009) Current Opinion in Biotechnology, 20:685-691).

In some embodiments, an IgG4 variant of the present disclosure may be combined with an S228P mutation according to the EU or, Kabat numbering convention (Angal et al., (1993) Mol Immunol, 30:105-108) and/or with one or more mutations described in Peters et al., (2012) J Biol Chem. 13; 287(29):24525-33) to enhance antibody stabilization.

Exemplary Anti-TREM2 Antibodies

In some embodiments, an isolated anti-TREM2 antibody of the present disclosure enhances one or more TREM2 activities induced by binding of one or more TREM2 ligands to the TREM2 protein, as compared to the one or more TREM2 activities induced by binding of the one or more TREM2 ligands to the TREM2 protein in the absence of the isolated antibody. In some embodiments, the anti-TREM2 antibody enhances the one or more TREM2 activities without competing with or otherwise blocking binding of the one or more TREM2 ligands to the TREM2 protein. In some embodiments, the antibody is a human antibody, a humanized antibody, a bispecific antibody, a multivalent antibody, or a chimeric antibody. Exemplary descriptions of such antibodies are found throughout the present disclosure. In some embodiments, the antibody is a bispecific antibody recognizing a first antigen and a second antigen.

In some embodiments, anti-TREM2 antibodies of the present disclosure bind to a human TREM2, or a homolog thereof, including without limitation a mammalian (e.g., non-human mammalian) TREM2 protein, mouse TREM2 protein (Uniprot Accession No. Q99NH8), rat TREM2 protein (Uniprot Accession No. D3ZZ89), Rhesus monkey TREM2 protein (Uniprot Accession No. F6QVF2), bovine TREM2 protein (Uniprot Accession No. Q05B59), equine TREM2 protein (Uniprot Accession No. F7D6L0), pig TREM2 protein (Uniprot Accession No. H2EZZ3), and dog TREM2 protein (Uniprot Accession No. E2RP46). In some embodiments, anti-TREM2 antibodies of the present disclosure specifically bind to human TREM2. In some embodiments, anti-TREM2 antibodies of the present disclosure specifically bind to mouse TREM2. In some embodiments, anti-TREM2 antibodies of the present disclosure specifically bind to both human TREM2 and mouse TREM2. In some embodiments, anti-TREM2 antibodies of the present disclosure modulate (e.g., induce or inhibit) at least one TREM2 activity. In some embodiments, the at least one TREM2 activity includes, without limitation, (a) modulated expression of one or more anti-inflammatory mediators, optionally wherein the one or more anti-inflammatory mediators are selected from IL-4, IL-10 TGF-β, IL-13, IL-35 IL-16, IFN-alpha, IL-1Ra, VEGF, G-CSF, YM, AXL, FLT1, and soluble receptors for TNF or IL-6; (b) modulated expression of one or more anti-inflammatory mediators in one or more cells selected from macrophages, dendritic cells, bone marrow-derived dendritic cells, monocytes, osteoclasts, and microglial cells; (c) modulated expression of one or more pro-inflammatory mediators, optionally wherein the one or more pro-inflammatorymediators are selected from IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF1, OPN, CD11c, GM-CSF, IL-11, IL-12, IL-17, IL-18, and IL-23; modulation of one or more genes whose expression is increased upon induction of inflammation, optionally wherein the one or more genes are selected from the group consisting of Fabp3, Fabp5, and LDR; modulating secretion of one or more pro-inflammatory mediators selected from IFN-β, IL-1α, IL-1β, CD86, TNF-α, IL-6, IL-8, CRP, MCP-1/CCL2, CCL3, CCL4, CCL5, CCR2, CXCL-10, Gata3, IL-20 family members, IL-33, LIF, IFN-gamma, OSM, CNTF, CSF1, OPN, CD11c, GM-CSF, IL-11, IL-12, IL-17, IL-18, and IL-23, and optionally where the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; modulating secretion of one or more anti-inflammatory mediators selected from IL-4, IL-10 TGF-β, IL-13, IL-35 IL-16, IFN-alpha, IL-1Ra, VEGF, G-CSF, YM, AXL, FLT1, and soluble receptors for TNF or IL-6, and optionally where the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; (d) modulated expression of one or more pro-inflammatory mediators in one or more cells selected from macrophages, dendritic cells, bone marrow-derived dendritic cells, monocytes, osteoclasts, and microglial cells; (e) activation of extracellular signal-regulated kinase (ERK) phosphorylation; (f) activating tyrosine phosphorylation on multiple cellular proteins; (g) modulated expression of C-C chemokine receptor 7 (CCR7); (h) activation of microglial cell chemotaxis toward CCL19 and CCL21 expressing cells; (i) increasing T cell priming and/or modulated T cell function of CD8+ T cells, CD4+ T cells, and/or regulatory T cells induced by one or more cells selected from dendritic cells, bone marrow-derived dendritic cells, monocytes, microglia, M1 microglia, activated M1 microglia, M2 microglia, macrophages, M1 macrophages, activated M1 macrophages, and M2 macrophages; (j) activation of osteoclast production, increased rate of rate of osteoclastogenesis, or both; (k) increased survival of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (l) increased proliferation of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (m) activating migration of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (n) activating one or more functions of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (o) activating maturation of one or more cells selected from dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, neutrophils, microglia, M1 microglia, activated M1 microglia, and M2 microglia; (p) activating of one or more types of clearance selected from apoptotic neuron clearance, nerve tissue debris clearance, non-nerve tissue debris clearance, bacteria clearance, other foreign body clearance, disease-causing protein clearance, disease-causing peptide clearance, and tumor cell clearance; optionally wherein the disease-causing protein is selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides and the tumor cell is from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer; (u) activation of phagocytosis of one or more of apoptotic neurons, nerve tissue debris, non-nerve tissue debris, bacteria, other foreign bodies, disease-causing proteins, disease-causing peptides, disease-causing nucleic acids, or tumor cells; optionally wherein the disease-causing nucleic acids are antisense GGCCCC (G2C4) repeat-expansion RNA, the disease-causing proteins are selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the tumor cells are from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, or thyroid cancer; (p) binding to TREM2 ligand on tumor cells; (q) binding to TREM2 ligand on cells selected from neutrophils, dendritic cells, bone marrow-derived dendritic cells, monocytes, microglia, and macrophages; (r) activation of tumor cell killing by one or more of microglia, macrophages, dendritic cells, bone marrow-derived dendritic cells, neutrophils, T cells, T helper cells, or cytotoxic T cells; (s) activating anti-tumor cell proliferation activity of one or more of microglia, macrophages, dendritic cells, bone marrow-derived dendritic cells, neutrophils, T cells, T helper cells, or cytotoxic T cells; (t) activating anti-tumor cell metastasis activity of one or more of microglia, macrophages, dendritic cells, bone marrow-derived dendritic cells, neutrophils, T cells, T helper cells, or cytotoxic T cells; (y) activating of one or more ITAM motif containing receptors, optionally wherein the one or more ITAM motif containing receptors are selected from TREM1, TREM2, FcgR, DAP10, and DAP12; (z) activating of signaling by one or more pattern recognition receptors (PRRs), optionally wherein the one or more PRRs are selected from receptors that identify pathogen-associated molecular patterns (PAMPs), receptors that identify damage-associated molecular patterns (DAMPs), and any combination thereof; (aa) activating of one or more receptors comprising the motif D/Ex₀₋₂YxxL/IX₆₋₈YxxL/I (SEQ ID NO: 883); (bb) activating of signaling by one or more Toll-like receptors; (cc) activating of the JAK-STAT signaling pathway; (dd) activating of nuclear factor kappa-light-chain-enhancer of activated B cells (NFκB); (dd) phosphorylation of an ITAM motif containing receptor; (ee) modulated expression of one or more inflammatory receptors, optionally wherein the one or more inflammatory receptors comprise CD86 and the one or more inflammatory receptors are expressed on one or more of microglia, macrophages, dendritic cells, bone marrow-derived dendritic cells, neutrophils, T cells, T helper cells, or cytotoxic T cells; (ff) increasing expression of one or more TREM2-dependent genes; (gg) normalization of disrupted TREM2-dependent gene expression; (hh) increasing expression of one or more ITAM-dependent genes, optionally wherein the one more ITAM-dependent genes are activated by nuclear factor of activated T cells (NFAT) transcription factors; (ii) inhibiting differentiation of one or more of immunosuppressor dendritic cells, immunosuppressor macrophages, myeloid derived suppressor cells, tumor-associated macrophages, immunosuppressor neutrophils, and regulatory T cells; (jj) inhibiting functionality of one or more of immunosuppressor dendritic cells, immunosuppressor macrophages, myeloid-derived suppressor cells, tumor-associated macrophages, immunosuppressor neutrophils, and regulatory T cells; (kk) decreasing infiltration of one or more of immunosuppressor dendritic cells, immunosuppressor macrophages, myeloid derived suppressor cells, tumor-associated macrophages, immunosuppressor neutrophils, and regulatory T cells into tumors; (11) decreasing number of tumor-promoting myeloid/granulocytic immune-suppressive cells in a tumor, in peripheral blood, or other lymphoid organ; (mm) inhibiting tumor-promoting activity of myeloid-derived suppressor cells; (nn) decreasing expression of tumor-promoting cytokines in a tumor or in peripheral blood, optionally wherein the tumor-promoting cytokines are TGF-beta or IL-10; (oo) decreasing tumor infiltration of tumor-promoting FoxP3+ regulatory T lymphocytes; (pp) increasing activation of tumor-specific T lymphocytes with tumor killing potential; (qq) decreasing tumor volume; (rr) decreasing tumor growth rate; (ss) increasing efficacy of one or more immune-therapies that modulate anti-tumor T cell responses, optionally wherein the one or more immune-therapies are selected from PD1/PDL1 blockade, CTLA-4 blockade, and cancer vaccines; (tt) inhibition of PLCγ/PKC/calcium mobilization; and (uu) inhibition of PI3K/Akt, Ras/MAPK signaling. (vv) increasing phagocytosis by dendritic cells, macrophages, monocytes, and/or microglia (ww) induction or retaintion of TREM2 clustering on a cell surface; (xx) TREM2 binding to DAP12; (yy) TREM2 phosphorylation; (zz) DAP12 phosphorylation; (aaa) activation of one or more SRC family tyrosine kinases including Syk kinase; (bbb) recruitment of Syk, ZAP70, or both to a DAP12/TREM2 complex; (ccc) modulating expression of one or more proteins selected from C1qa, C1qB, C1qC, C1s, C1R, C4, C2, C3, ITGB2, HMOX1, LAT2. CASP1, CSTA, VSIG4, MS4A4A, C3AR1, GPX1, TyroBP, ALOXSAP, ITGAM, SLC7A7, CD4, ITGAX, PYCARD, and VEGF; (ddd) increasing memory; and (eee) reducing cognitive deficit.

In some embodiments, anti-TREM2 antibodies of the present disclosure bind to membrane bound or soluble form of a TREM2 protein of the present disclosure and/or naturally occurring variants. In certain preferred embodiments, the anti-TREM2 antibodies bind to human TREM2.

In some embodiments, anti-TREM2 antibodies of the present disclosure are agonist antibodies or antagonist antibodies that bind to a TREM2 protein of the present disclosure expressed on the surface of a cell and modulate (e.g., induce or inhibit) at least one TREM2 activity of the present disclosure after binding to the surface-expressed TREM2 protein. In some embodiments, anti-TREM2 antibodies of the present disclosure are inert antibodies.

Anti-TREM2 Antibody-Binding Regions

Certain aspects of the preset disclosure provide anti-TREM2 antibodies that bind to one or more amino acids within amino acid residues 19-174; 29-112; 113-174; 35-49, 35-49 and 140-150; 39-49, 39-49 and 63-77; 51-61; 55-62; 55-62, 104-109, and 148-158; 55-62, 104-109, and 160-166; 55-65, 55-65 and 124-134; 63-73; 63-77; 104-109; 117-133; 124-134; 137-146; 139-147; 139-149; 140-150; 140-146; 140-143; 142-152; 146-154; 148-158; 149-157; 149 and 150; 151-155; 154-161; 156-170; 160-166; or 162-165 of human TREM2 (SEQ ID NO: 1), or within amino acid residues on a TREM2 homolog or ortholog corresponding to amino acid residues 19-174; 29-112; 113-174; 35-49, 35-49 and 140-150; 39-49, 39-49 and 63-77; 51-61; 55-62; 55-62, 104-109, and 148-158; 55-62, 104-109, and 160-166; 55-65, 55-65 and 124-134; 63-73; 63-77; 104-109; 117-133; 124-134; 137-146; 139-147; 139-149; 140-150; 140-146; 140-143; 142-152; 146-154; 148-158; 149-157; 149 and 150; 151-155; 154-161; 156-170; 160-166; or 162-165 of SEQ ID NO: 1. In some embodiments, the anti-TREM2 antibody binds to one or more amino acids within amino acid residues 35-49 of human TREM2 (SEQ ID NO: 1), or within amino acid residues on a TREM2 homolog or ortholog corresponding to amino acid residues 35-49 of SEQ ID NO: 1. In some embodiments, the anti-TREM2 antibody binds to one or more amino acids within amino acid residues 35-49 and 140-150 of human TREM2 (SEQ ID NO: 1), or within amino acid residues on a TREM2 homolog or ortholog corresponding to amino acid residues 35-49 and 140-150 of SEQ ID NO: 1. In some embodiments, the anti-TREM2 antibody binds to one or more amino acids within amino acid residues 39-49 of human TREM2 (SEQ ID NO: 1), or within amino acid residues on a TREM2 homolog or ortholog corresponding to amino acid residues 39-49 of SEQ ID NO: 1. In some embodiments, the anti-TREM2 antibody binds to one or more amino acids within amino acid residues 39-49 and 63-77 of human TREM2 (SEQ ID NO: 1), or within amino acid residues on a TREM2 homolog or ortholog corresponding to amino acid residues 39-49 and 63-77 of SEQ ID NO: 1. In some embodiments, the anti-TREM2 antibody binds to one or more amino acids within amino acid residues 51-61 of human TREM2 (SEQ ID NO: 1), or within amino acid residues on a TREM2 homolog or ortholog corresponding to amino acid residues 51-61 of SEQ ID NO: 1. In some embodiments, the anti-TREM2 antibody binds to one or more amino acids within amino acid residues 55-62 of human TREM2 (SEQ ID NO: 1), or within amino acid residues on a TREM2 homolog or ortholog corresponding to amino acid residues 55-62 of SEQ ID NO: 1. In some embodiments, the anti-TREM2 antibody binds to one or more amino acids within amino acid residues 55-62, 104-109, and 148-158 and of human TREM2 (SEQ ID NO: 1), or within amino acid residues on a TREM2 homolog or ortholog corresponding to amino acid residues 55-62, 104-109, and 148-158 of SEQ ID NO: 1. In some embodiments, the anti-TREM2 antibody binds to one or more amino acids within amino acid residues 55-62, 104-109, and 160-166 and of human TREM2 (SEQ ID NO: 1), or within amino acid residues on a TREM2 homolog or ortholog corresponding to amino acid residues 55-62, 104-109, and 160-166 of SEQ ID NO: 1. In some embodiments, the anti-TREM2 antibody binds to one or more amino acids within amino acid residues 55-65 of human TREM2 (SEQ ID NO: 1), or within amino acid residues on a TREM2 homolog or ortholog corresponding to amino acid residues 55-65 of SEQ ID NO: 1. In some embodiments, the anti-TREM2 antibody binds to one or more amino acids within amino acid residues 55-65 and 124-134 of human TREM2 (SEQ ID NO: 1), or within amino acid residues on a TREM2 homolog or ortholog corresponding to amino acid residues 55-65 and 124-134 of SEQ ID NO: 1. In some embodiments, the anti-TREM2 antibody binds to one or more amino acids within amino acid residues 63-73 of human TREM2 (SEQ ID NO: 1), or within amino acid residues on a TREM2 homolog or ortholog corresponding to amino acid residues 63-73 of SEQ ID NO: 1. In some embodiments, the anti-TREM2 antibody binds to one or more amino acids within amino acid residues 63-77 of human TREM2 (SEQ ID NO: 1), or within amino acid residues on a TREM2 homolog or ortholog corresponding to amino acid residues 63-77 of SEQ ID NO: 1. In some embodiments, the anti-TREM2 antibody binds to one or more amino acids within amino acid residues 104-109 of human TREM2 (SEQ ID NO: 1), or within amino acid residues on a TREM2 homolog or ortholog corresponding to amino acid residues 104-109 of SEQ ID NO: 1. In some embodiments, the anti-TREM2 antibody binds to one or more amino acids within amino acid residues 117-133 of human TREM2 (SEQ ID NO: 1), or within amino acid residues on a TREM2 homolog or ortholog corresponding to amino acid residues 117-133 of SEQ ID NO: 1. In some embodiments, the anti-TREM2 antibody binds to one or more amino acids within amino acid residues 124-134 of human TREM2 (SEQ ID NO: 1), or within amino acid residues on a TREM2 homolog or ortholog corresponding to amino acid residues 124-134 of SEQ ID NO: 1.In some embodiments, the anti-TREM2 antibody binds to one or more amino acids within amino acid residues 137-146 of human TREM2 (SEQ ID NO: 1), or within amino acid residues on a TREM2 homolog or ortholog corresponding to amino acid residues 137-146 of SEQ ID NO: 1. In some embodiments, the anti-TREM2 antibody binds to one or more amino acids within amino acid residues 139-147 of human TREM2 (SEQ ID NO: 1), or within amino acid residues on a TREM2 homolog or ortholog corresponding to amino acid residues 139-147 of SEQ ID NO: 1. In some embodiments, the anti-TREM2 antibody binds to one or more amino acids within amino acid residues 139-149 of human TREM2 (SEQ ID NO: 1), or within amino acid residues on a TREM2 homolog or ortholog corresponding to amino acid residues 139-149 of SEQ ID NO: 1. In some embodiments, the anti-TREM2 antibody binds to one or more amino acids within amino acid residues 140-150 of human TREM2 (SEQ ID NO: 1), or within amino acid residues on a TREM2 homolog or ortholog corresponding to amino acid residues 140-150 of SEQ ID NO: 1. In some embodiments, the anti-TREM2 antibody binds to one or more amino acids within amino acid residues 140-146 of human TREM2 (SEQ ID NO: 1), or within amino acid residues on a TREM2 homolog or ortholog corresponding to amino acid residues 140-146 of SEQ ID NO: 1. In some embodiments, the anti-TREM2 antibody binds to one or more amino acids within amino acid residues 140-143 of human TREM2 (SEQ ID NO: 1), or within amino acid residues on a TREM2 homolog or ortholog corresponding to amino acid residues 140-143 of SEQ ID NO: 1. In some embodiments, the anti-TREM2 antibody binds to one or more amino acids within amino acid residues 142-152 of human TREM2 (SEQ ID NO: 1), or within amino acid residues on a TREM2 homolog or ortholog corresponding to amino acid residues 142-152 of SEQ ID NO: 1. In some embodiments, the anti-TREM2 antibody binds to one or more amino acids within amino acid residues 146-154 of human TREM2 (SEQ ID NO: 1), or within amino acid residues on a TREM2 homolog or ortholog corresponding to amino acid residues 146-154 of SEQ ID NO: 1. In some embodiments, the anti-TREM2 antibody binds to one or more amino acids within amino acid residues 148-158 of human TREM2 (SEQ ID NO: 1), or within amino acid residues on a TREM2 homolog or ortholog corresponding to amino acid residues 148-158 of SEQ ID NO: 1. In some embodiments, the anti-TREM2 antibody binds to one or more amino acids within amino acid residues 149-157 of human TREM2 (SEQ ID NO: 1), or within amino acid residues on a TREM2 homolog or ortholog corresponding to amino acid residues 149-157 of SEQ ID NO: 1. In some embodiments, the anti-TREM2 antibody binds to one or more amino acids within amino acid residues 149 and 150 of human TREM2 (SEQ ID NO: 1), or within amino acid residues on a TREM2 homolog or ortholog corresponding to amino acid residues 149 and 150 of SEQ ID NO: 1. In some embodiments, the anti-TREM2 antibody binds to one or more amino acids within amino acid residues 154-161 of human TREM2 (SEQ ID NO: 1), or within amino acid residues on a TREM2 homolog or ortholog corresponding to amino acid residues 154-161 of SEQ ID NO: 1. In some embodiments, the anti-TREM2 antibody binds to one or more amino acids within amino acid residues 156-170 of human TREM2 (SEQ ID NO: 1), or within amino acid residues on a TREM2 homolog or ortholog corresponding to amino acid residues 156-170 of SEQ ID NO: 1. In some embodiments, the anti-TREM2 antibody binds to one or more amino acids within amino acid residues 160-166 of human TREM2 (SEQ ID NO: 1), or within amino acid residues on a TREM2 homolog or ortholog corresponding to amino acid residues 160-166 of SEQ ID NO: 1. In some embodiments, the anti-TREM2 antibody binds to one or more amino acids within amino acid residues 162-165 of human TREM2 (SEQ ID NO: 1), or within amino acid residues on a TREM2 homolog or ortholog corresponding to amino acid residues 162-165 of SEQ ID NO: 1.

In other embodiments, anti-TREM2 antibodies of the present disclosure bind to an epitope that includes amino acid residue Arg47 or Asp87 of human TREM 2 (SEQ ID NO: 1). In some embodiments, anti-TREM2 antibodies of the present disclosure bind to an epitope that includes amino acid residues 40-44 of human TREM 2 (SEQ ID NO: 1). In some embodiments, anti-TREM2 antibodies of the present disclosure bind to an epitope that includes amino acid residues 67-76 of human TREM 2 (SEQ ID NO: 1). In some embodiments, anti-TREM2 antibodies of the present disclosure bind to an epitope that includes amino acid residues 114-118 of human TREM 2 (SEQ ID NO: 1).

In some embodiments, an anti-TREM2 antibody of the present disclosure binds to one or more amino acid residues selected from K42, H43, W44, G45, H67, R77, T88, H114, E117, E151, D152, H154, and E156 of SEQ ID NO: 1, or one or more amino acid residues on a mammalian TREM2 protein corresponding to an amino acid residue selected from K42, H43, W44, G45, H67, R77, T88, H114, E117, E151, D152, H154, and E156 of SEQ ID NO: 1. In some embodiments, an anti-TREM2 antibody of the present disclosure binds to one or more, two or more, three or more, or all four amino acid residues selected from E151, D152, H154, and E156 of SEQ ID NO: 1, or one or more, two or more, three or more, or all four amino acid residues on a mammalian TREM2 protein corresponding to an amino acid residue selected from E151, D152, H154, and E156 of SEQ ID NO: 1. In some embodiments, an anti-TREM2 antibody of the present disclosure binds to one or more or all two amino acid residues selected from K42 and H114 of SEQ ID NO: 1, or one or more, or all two amino acid residues on a mammalian TREM2 protein corresponding to an amino acid residue selected from K42 and H114 of SEQ ID NO: 1. In some embodiments, an anti-TREM2 antibody of the present disclosure binds to one or more, two or more, or all three amino acid residues selected from K42, G45, and H114 of SEQ ID NO: 1, or one or more, two or more, or all three amino acid residues on a mammalian TREM2 protein corresponding to an amino acid residue selected from K42, G45, and H114 of SEQ ID NO: 1. In some embodiments, an anti-TREM2 antibody of the present disclosure binds to the amino acid residue R77 of SEQ ID NO: 1, or an amino acid residue on a mammalian TREM2 protein corresponding to the amino acid residue R77 of SEQ ID NO: 1.

In some embodiments, anti-TREM2 antibodies of the present disclosure competitively inhibit binding of at least one antibody selected from any of the antibodies listed in Tables 2A, 2B, 3A, 3B, 4A, 4B, 7A, and 7B. In some embodiments, anti-TREM2 antibodies of the present disclosure competitively inhibit binding of at least one antibody selected from 11A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2.

In some embodiments, anti-TREM2 antibodies of the present disclosure bind to an epitope of human TREM2 that is the same as or overlaps with the TREM2 epitope bound by at least one antibody selected from any of the antibodies listed in Tables 2A, 2B, 3A, 3B, 4A, 4B, 7A, and 7B. In some embodiments, anti-TREM2 antibodies of the present disclosure bind to an epitope of human TREM2 that is the same as or overlaps with the TREM2 epitope bound by at least one antibody selected from 11A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2.

In some embodiments, anti-TREM2 antibodies of the present disclosure bind essentially the same TREM2 epitope bound by at least one antibody selected from any of the antibodies listed in Tables 2A, 2B, 3A, 3B, 4A, 4B, 7A, and 7B. In some embodiments, anti-TREM2 antibodies of the present disclosure bind essentially the same TREM2 epitope bound by at least one antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In some embodiments, anti-TREM2 antibodies of the present disclosure compete with one or more antibodies selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2, and any combination thereof for binding to TREM2.

In an exemplary competition assay, immobilized TREM2 or cells expressing TREM2 on the cell surface are incubated in a solution comprising a first labeled antibody that binds to TREM2 (e.g., human or non-human primate) and a second unlabeled antibody that is being tested for its ability to compete with the first antibody for binding to TREM2. The second antibody may be present in a hybridoma supernatant. As a control, immobilized TREM2 or cells expressing TREM2 is incubated in a solution comprising the first labeled antibody but not the second unlabeled antibody. After incubation under conditions permissive for binding of the first antibody to TREM2, excess unbound antibody is removed, and the amount of label associated with immobilized TREM2 or cells expressing TREM2 is measured. If the amount of label associated with immobilized TREM2 or cells expressing TREM2 is substantially reduced in the test sample relative to the control sample, then that indicates that the second antibody is competing with the first antibody for binding to TREM2. See, Harlow and Lane (1988) Antibodies: A Laboratory Manual ch.14 (Cold Spring Harbor Laboratory, Cold Spring Harbor, N.Y.).

Anti-TREM2 Antibody Light Chain and Heavy Chain Variable Regions

In some embodiments, anti-TREM2 antibodies of the present disclosure comprise (a) a light chain variable region comprising at least one, two, or three HVRs selected from HVR-L1, HVR-L2, and HVR-L3 of any one of the antibodies listed in Tables 2A, 2B, 3A, 3B, 4A, 4B, 7A, and 7B, or selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2, and any combination thereof; and/or (b) a heavy chain variable region comprising at least one, two, or three HVRs selected from HVR-H1, HVR-H2, and HVR-H3 of any one of the antibodies listed in Tables 2A, 2B, 3A, 3B, 4A, 4B, 7A, and 7B, or selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2, and any combination thereof. In some embodiments, the HVR-L1, HVR-L2, HVR-L3, HVR-H1, HVR-H2, and HVR-H3 comprise EU or Kabat HVR, Chothia HVR, or Contact HVR sequences as shown in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2, and any combination thereof.

In some embodiments, anti-TREM2 antibodies of the present disclosure comprise at least one, two, three, four, five, or six HVRs selected from (i) HVR-L1 comprising the amino acid sequence of any of the HVR-L1 sequences listed in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; (ii) HVR-L2 comprising the amino acid sequence of any of the HVR-L2 sequences listed in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; (iii) HVR-L3 comprising the amino acid sequence of any of the HVR-L3 sequences listed in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; (iv) HVR-H1 comprising the amino acid sequence of any of the HVR-H1 sequences listed in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; (v) HVR-H2 comprising the amino acid sequence of any of the HVR-H2 sequences listed in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; and (vi) HVR-H3 comprising the amino acid sequence of any of the HVR-H3 sequences listed in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2. In some embodiments, anti-TREM2 antibodies of the present disclosure comprise a light chain variable domain and a heavy chain variable domain, wherein (a) the HVR-L1 comprises the amino acid sequence of SEQ ID NO: 9, the HVR-L2 comprises the amino acid sequence of SEQ ID NO: 24, the HVR-L3 comprises the amino acid sequence of SEQ ID NO: 34, the HVR-H1 comprises the amino acid sequence of SEQ ID NO: 48, the HVR-H2 comprises the amino acid sequence of SEQ ID NO: 66, and the HVR-H3 comprises the amino acid sequence of SEQ ID NO: 85; (b) the HVR-L1 comprises the amino acid sequence of SEQ ID NO: 9, the HVR-L2 comprises the amino acid sequence of SEQ ID NO: 24, the HVR-L3 comprises the amino acid sequence of SEQ ID NO: 34, the HVR-H1 comprises the amino acid sequence of SEQ ID NO: 48, the HVR-H2 comprises the amino acid sequence of SEQ ID NO: 66, and the HVR-H3 comprises the amino acid sequence of SEQ ID NO: 85; (c) the HVR-L1 comprises the amino acid sequence of SEQ ID NO: 10, the HVR-L2 comprises the amino acid sequence of SEQ ID NO: 25, the HVR-L3 comprises the amino acid sequence of SEQ ID NO: 35, the HVR-H1 comprises the amino acid sequence of SEQ ID NO: 49, the HVR-H2 comprises the amino acid sequence of SEQ ID NO: 67, and the HVR-H3 comprises the amino acid sequence of SEQ ID NO: 86; (d) the HVR-L1 comprises the amino acid sequence of SEQ ID NO: 12, the HVR-L2 comprises the amino acid sequence of SEQ ID NO: 26, the HVR-L3 comprises the amino acid sequence of SEQ ID NO: 37, the HVR-H1 comprises the amino acid sequence of SEQ ID NO: 50, the HVR-H2 comprises the amino acid sequence of SEQ ID NO: 68, and the HVR-H3 comprises the amino acid sequence of SEQ ID NO: 87; (e) the HVR-L1 comprises the amino acid sequence of SEQ ID NO: 11, the HVR-L2 comprises the amino acid sequence of SEQ ID NO: 26, the HVR-L3 comprises the amino acid sequence of SEQ ID NO: 36, the HVR-H1 comprises the amino acid sequence of SEQ ID NO: 51, the HVR-H2 comprises the amino acid sequence of SEQ ID NO: 69, and the HVR-H3 comprises the amino acid sequence of SEQ ID NO: 88; (f) the HVR-L1 comprises the amino acid sequence of SEQ ID NO: 13, the HVR-L2 comprises the amino acid sequence of SEQ ID NO: 27, the HVR-L3 comprises the amino acid sequence of SEQ ID NO: 38, the HVR-H1 comprises the amino acid sequence of SEQ ID NO: 52, the HVR-H2 comprises the amino acid sequence of SEQ ID NO: 70, and the HVR-H3 comprises the amino acid sequence of SEQ ID NO: 89; (g) the HVR-L1 comprises the amino acid sequence of SEQ ID NO: 14, the HVR-L2 comprises the amino acid sequence of SEQ ID NO: 28, the HVR-L3 comprises the amino acid sequence of SEQ ID NO: 39, the HVR-H1 comprises the amino acid sequence of SEQ ID NO: 53, the HVR-H2 comprises the amino acid sequence of SEQ ID NO: 71, and the HVR-H3 comprises the amino acid sequence of SEQ ID NO: 90; (h) the HVR-L1 comprises the amino acid sequence of SEQ ID NO: 13, the HVR-L2 comprises the amino acid sequence of SEQ ID NO: 27, the HVR-L3 comprises the amino acid sequence of SEQ ID NO: 38, the HVR-H1 comprises the amino acid sequence of SEQ ID NO: 52, the HVR-H2 comprises the amino acid sequence of SEQ ID NO: 70, and the HVR-H3 comprises the amino acid sequence of SEQ ID NO: 89; (i) the HVR-L1 comprises the amino acid sequence of SEQ ID NO: 13, the HVR-L2 comprises the amino acid sequence of SEQ ID NO: 27, the HVR-L3 comprises the amino acid sequence of SEQ ID NO: 38, the HVR-H1 comprises the amino acid sequence of SEQ ID NO: 52, the HVR-H2 comprises the amino acid sequence of SEQ ID NO: 70, and the HVR-H3 comprises the amino acid sequence of SEQ ID NO: 89; (j) the HVR-L1 comprises the amino acid sequence of SEQ ID NO: 15, the HVR-L2 comprises the amino acid sequence of SEQ ID NO: 28, the HVR-L3 comprises the amino acid sequence of SEQ ID NO: 40, the HVR-H1 comprises the amino acid sequence of SEQ ID NO: 54, the HVR-H2 comprises the amino acid sequence of SEQ ID NO: 72, and the HVR-H3 comprises the amino acid sequence of SEQ ID NO: 91; (k) the HVR-L1 comprises the amino acid sequence of SEQ ID NO: 11, the HVR-L2 comprises the amino acid sequence of SEQ ID NO: 26, the HVR-L3 comprises the amino acid sequence of SEQ ID NO: 36, the HVR-H1 comprises the amino acid sequence of SEQ ID NO: 51, the HVR-H2 comprises the amino acid sequence of SEQ ID NO: 69, and the HVR-H3 comprises the amino acid sequence of SEQ ID NO: 88; (1) the HVR-L1 comprises the amino acid sequence of SEQ ID NO: 16, the HVR-L2 comprises the amino acid sequence of SEQ ID NO: 29, the HVR-L3 comprises the amino acid sequence of SEQ ID NO: 35, the HVR-H1 comprises the amino acid sequence of SEQ ID NO: 55, the HVR-H2 comprises the amino acid sequence of SEQ ID NO: 73, and the HVR-H3 comprises the amino acid sequence of SEQ ID NO: 92; (m) the HVR-L1 comprises the amino acid sequence of SEQ ID NO: 15, the HVR-L2 comprises the amino acid sequence of SEQ ID NO: 28, the HVR-L3 comprises the amino acid sequence of SEQ ID NO: 40, the HVR-H1 comprises the amino acid sequence of SEQ ID NO: 54, the HVR-H2 comprises the amino acid sequence of SEQ ID NO: 72, and the HVR-H3 comprises the amino acid sequence of SEQ ID NO: 91; (n) the HVR-L1 comprises the amino acid sequence of SEQ ID NO: 581, the HVR-L2 comprises the amino acid sequence of SEQ ID NO: 29, the HVR-L3 comprises the amino acid sequence of SEQ ID NO: 582, the HVR-H1 comprises the amino acid sequence of SEQ ID NO: 56, the HVR-H2 comprises the amino acid sequence of SEQ ID NO: 74, and the HVR-H3 comprises the amino acid sequence of SEQ ID NO: 93; (o) the HVR-L1 comprises the amino acid sequence of SEQ ID NO: 17, the HVR-L2 comprises the amino acid sequence of SEQ ID NO: 30, the HVR-L3 comprises the amino acid sequence of SEQ ID NO: 41, the HVR-H1 comprises the amino acid sequence of SEQ ID NO: 57, the HVR-H2 comprises the amino acid sequence of SEQ ID NO: 75, and the HVR-H3 comprises the amino acid sequence of SEQ ID NO: 94; (p) the HVR-H1 comprises the amino acid sequence of SEQ ID NO: 58, the HVR-H2 comprises the amino acid sequence of SEQ ID NO: 76, and the HVR-H3 comprises the amino acid sequence of SEQ ID NO: 95; (q) the HVR-L1 comprises the amino acid sequence of SEQ ID NO: 18, the HVR-L2 comprises the amino acid sequence of SEQ ID NO: 31, the HVR-L3 comprises the amino acid sequence of SEQ ID NO: 42, the HVR-H1 comprises the amino acid sequence of SEQ ID NO: 59, the HVR-H2 comprises the amino acid sequence of SEQ ID NO: 77, and the HVR-H3 comprises the amino acid sequence of SEQ ID NO: 96; (r) the HVR-L1 comprises the amino acid sequence of SEQ ID NO: 19, the HVR-L2 comprises the amino acid sequence of SEQ ID NO: 28, the HVR-L3 comprises the amino acid sequence of SEQ ID NO: 43, the HVR-H1 comprises the amino acid sequence of SEQ ID NO: 60, the HVR-H2 comprises the amino acid sequence of SEQ ID NO: 78, and the HVR-H3 comprises the amino acid sequence of SEQ ID NO: 97; (s) the HVR-L1 comprises the amino acid sequence of SEQ ID NO: 20, the HVR-L2 comprises the amino acid sequence of SEQ ID NO: 28, the HVR-L3 comprises the amino acid sequence of SEQ ID NO: 44, the HVR-H1 comprises the amino acid sequence of SEQ ID NO: 61, the HVR-H2 comprises the amino acid sequence of SEQ ID NO: 79, and the HVR-H3 comprises the amino acid sequence of SEQ ID NO: 98; (t) the HVR-L1 comprises the amino acid sequence of SEQ ID NO: 21, the HVR-L2 comprises the amino acid sequence of SEQ ID NO: 32, the HVR-L3 comprises the amino acid sequence of SEQ ID NO: 45, the HVR-H1 comprises the amino acid sequence of SEQ ID NO: 62, the HVR-H2 comprises the amino acid sequence of SEQ ID NO: 80, and the HVR-H3 comprises the amino acid sequence of SEQ ID NO: 99; (u) the HVR-L1 comprises the amino acid sequence of SEQ ID NO: 15, the HVR-L2 comprises the amino acid sequence of SEQ ID NO: 33, the HVR-L3 comprises the amino acid sequence of SEQ ID NO: 40, the HVR-H1 comprises the amino acid sequence of SEQ ID NO: 54, the HVR-H2 comprises the amino acid sequence of SEQ ID NO: 81, and the HVR-H3 comprises the amino acid sequence of SEQ ID NO: 91; (v) the HVR-L1 comprises the amino acid sequence of SEQ ID NO: 22, the HVR-L2 comprises the amino acid sequence of SEQ ID NO: 29, the HVR-L3 comprises the amino acid sequence of SEQ ID NO: 46, the HVR-H1 comprises the amino acid sequence of SEQ ID NO: 63, the HVR-H2 comprises the amino acid sequence of SEQ ID NO: 82, and the HVR-H3 comprises the amino acid sequence of SEQ ID NO: 100; (w) the HVR-L1 comprises the amino acid sequence of SEQ ID NO: 23, the HVR-L2 comprises the amino acid sequence of SEQ ID NO: 29, the HVR-L3 comprises the amino acid sequence of SEQ ID NO: 47, the HVR-H1 comprises the amino acid sequence of SEQ ID NO: 64, the HVR-H2 comprises the amino acid sequence of SEQ ID NO: 83, and the HVR-H3 comprises the amino acid sequence of SEQ ID NO: 101; (x) the HVR-L1 comprises the amino acid sequence of SEQ ID NO: 16, the HVR-L2 comprises the amino acid sequence of SEQ ID NO: 29, the HVR-L3 comprises the amino acid sequence of SEQ ID NO: 35, the HVR-H1 comprises the amino acid sequence of SEQ ID NO: 65, the HVR-H2 comprises the amino acid sequence of SEQ ID NO: 84, and the HVR-H3 comprises the amino acid sequence of SEQ ID NO: 102, (y) the HVR-L1 comprises the amino acid sequence of SEQ ID NO: 581, the HVR-L2 comprises the amino acid sequence of SEQ ID NO: 29, the HVR-L3 comprises the amino acid sequence of SEQ ID NO: 582, the HVR-H1 comprises the amino acid sequence of SEQ ID NO: 56, the HVR-H2 comprises the amino acid sequence of SEQ ID NO: 585, and the HVR-H3 comprises the amino acid sequence of SEQ ID NO: 588, (z) the HVR-L1 comprises the amino acid sequence of SEQ ID NO: 10, the HVR-L2 comprises the amino acid sequence of SEQ ID NO: 29, the HVR-L3 comprises the amino acid sequence of SEQ ID NO: 35, the HVR-H1 comprises the amino acid sequence of SEQ ID NO: 49, the HVR-H2 comprises the amino acid sequence of SEQ ID NO: 586, and the HVR-H3 comprises the amino acid sequence of SEQ ID NO: 86, or (aa) the HVR-L1 comprises the amino acid sequence of SEQ ID NO: 14, the HVR-L2 comprises the amino acid sequence of SEQ ID NO: 28, the HVR-L3 comprises the amino acid sequence of SEQ ID NO: 583, the HVR-H1 comprises the amino acid sequence of SEQ ID NO: 584, the HVR-H2 comprises the amino acid sequence of SEQ ID NO: 587, and the HVR-H3 comprises the amino acid sequence of SEQ ID NO: 589.

In some embodiments, anti-TREM2 antibodies of the present disclosure comprise at least one, two, three, four, five, or six HVRs selected from (i) HVR-L1 comprising an amino acid sequence selected from SEQ ID NOs: 826-828, or an amino acid sequence with at least about 90% homology to an amino acid sequence selected from SEQ ID NOs: 826-828; (ii) HVR-L2 comprising the amino acid sequence of any of the HVR-L2 sequences listed in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; (iii) HVR-L3 comprising the amino acid sequence of any of the HVR-L3 sequences listed in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; (iv) HVR-H1 comprising an amino acid sequence selected from SEQ ID NOs: 829-835, or an amino acid sequence with at least about 90% homology to an amino acid sequence selected from SEQ ID NOs: 829-835; (v) HVR-H2 comprising an amino acid sequence selected from SEQ ID NOs: 836-842, or an amino acid sequence with at least about 90% homology to an amino acid sequence selected from SEQ ID NOs: 836-842; and (vi) HVR-H3 comprising the amino acid sequence of any of the HVR-H3 sequences listed in Tables 2A, 2B, 3A, 3B, 4A, 4B, 7A, and 7B, or from an antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2.

In some embodiments, anti-TREM2 antibodies of the present disclosure comprise a light chain variable domain and a heavy chain variable domain, wherein the light chain variable domain comprises one or more of: (a) an HVR-L1 comprising an amino acid sequence selected from SEQ ID NOs: 9-23, 581, 690-694, 734-738, and 826-828, or an amino acid sequence with at least about 90% homology to an amino acid sequence selected from SEQ ID NOs: 9-23, 581, 690-694, 734-738, and 826-828; (b) an HVR-L2 comprising an amino acid sequence selected from SEQ ID NOs: 24-33, 695-697, and 739-743, or an amino acid sequence with at least about 90% homology to an amino acid sequence selected from SEQ ID NOs: 24-33, 695-697, and 739-743; and (c) an HVR-L3 comprising an amino acid sequence selected from SEQ ID NOs: 34-47, 582, 583, 698-702, and 744-746, or an amino acid sequence with at least about 90% homology to an amino acid sequence selected from SEQ ID NOs: 34-47, 582, 583, 698-702, and 744-746; and/or wherein the heavy chain variable domain comprises one or more of: (a) an HVR-H1 comprising an amino acid sequence selected from SEQ ID NOs: 48-65, 584, 703-705, 747-754, and 829-835, or an amino acid sequence with at least about 90% homology to an amino acid sequence selected from SEQ ID NOs: 48-65, 584, 703-705, 747-754, and 829-835; (b) an HVR-H2 comprising an amino acid sequence selected from SEQ ID NOs: 66-84, 585-587, 706-708, 755-762, 836-842, and 888, or an amino acid sequence with at least about 90% homology to an amino acid sequence selected from SEQ ID NOs: 66-84, 585-587, 706-708, 755-762, 836-842, and 888; and (c) an HVR-H3 comprising an amino acid sequence selected from SEQ ID NOs: 85-102, 588, 589, 709, 710, and 763-770, or an amino acid sequence with at least about 90% homology to an amino acid sequence selected from SEQ ID NOs: 85-102, 588, 589, 709, 710, and 763-770.

In some embodiments, anti-TREM2 antibodies of the present disclosure comprise a light chain variable region of any one of the antibodies listed in Tables 2A, 2B, 3A, 3B, 4A, 4B, 7A, and 7B, or selected from 1A7, 3A2, 3B10, 6G12, 6H6, 7A9, 7B3, 8A1, 8E10, 8F11, 8F8, 9F5, 9F5v2, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7E5v2, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2; and/or a heavy chain variable region of any one of the antibodies listed in Tables 2A, 2B, 3A, 3B, 4A, 4B, 7A, and 7B, or selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2. In some embodiments, anti-TREM2 antibodies of the present disclosure comprise a light chain variable region comprising an amino acid sequence selected from any of SEQ ID NOs: 219-398, 602-634, 679-689, 724-730, 809-816, 821, 843, 844, 849, and 850; and/or a heavy chain variable domain comprising an amino acid sequence selected from any of SEQ ID NOs: 399-580, 635-678, 731-733, and 817-820, 822-825, and 845-847. In some embodiments, the antibody comprises a light chain variable domain comprising the amino acid sequence of SEQ ID NO:843 and a heavy chain variable domain comprising the amino acid sequence of SEQ ID NO:845. In some embodiments, the antibody comprises a light chain variable domain comprising the amino acid sequence of SEQ ID NO:843 and a heavy chain variable domain comprising the amino acid sequence of SEQ ID NO:846. In some embodiments, the antibody comprises a light chain variable domain comprising the amino acid sequence of SEQ ID NO:843 and a heavy chain variable domain comprising the amino acid sequence of SEQ ID NO:847. In some embodiments, the antibody comprises a light chain variable domain comprising the amino acid sequence of SEQ ID NO:844 and a heavy chain variable domain comprising the amino acid sequence of SEQ ID NO:845. In some embodiments, the antibody comprises a light chain variable domain comprising the amino acid sequence of SEQ ID NO:844 and a heavy chain variable domain comprising the amino acid sequence of SEQ ID NO:846. In some embodiments, the antibody comprises a light chain variable domain comprising the amino acid sequence of SEQ ID NO:844 and a heavy chain variable domain comprising the amino acid sequence of SEQ ID NO:847. In some embodiments, the antibody comprises a light chain variable domain and a heavy chain variable domain, wherein: (a) the light chain variable domain comprises the amino acid sequence of SEQ ID NO: 333 and the heavy chain variable domain comprises the amino acid sequence of SEQ ID NO:521; (b) the light chain variable domain comprises the amino acid sequence of SEQ ID NO: 850 and the heavy chain variable domain comprises the amino acid sequence of SEQ ID NO:521; (c) the light chain variable domain comprises the amino acid sequence of SEQ ID NO: 334 and the heavy chain variable domain comprises the amino acid sequence of SEQ ID NO:522; (d) the light chain variable domain comprises the amino acid sequence of SEQ ID NO: 335 and the heavy chain variable domain comprises the amino acid sequence of SEQ ID NO:523; (e) the light chain variable domain comprises the amino acid sequence of SEQ ID NO: 336 and the heavy chain variable domain comprises the amino acid sequence of SEQ ID NO:524; (f) the light chain variable domain comprises the amino acid sequence of SEQ ID NO: 337 and the heavy chain variable domain comprises the amino acid sequence of SEQ ID NO:525; (g) the light chain variable domain comprises the amino acid sequence of SEQ ID NO: 338 and the heavy chain variable domain comprises the amino acid sequence of SEQ ID NO:526; (h) the light chain variable domain comprises the amino acid sequence of SEQ ID NO: 339 and the heavy chain variable domain comprises the amino acid sequence of SEQ ID NO:526; (i) the light chain variable domain comprises the amino acid sequence of SEQ ID NO: 340 and the heavy chain variable domain comprises the amino acid sequence of SEQ ID NO:527; (j) the light chain variable domain comprises the amino acid sequence of SEQ ID NO: 341 and the heavy chain variable domain comprises the amino acid sequence of SEQ ID NO:528; (k) the light chain variable domain comprises the amino acid sequence of SEQ ID NO: 342 and the heavy chain variable domain comprises the amino acid sequence of SEQ ID NO:529; (1) the light chain variable domain comprises the amino acid sequence of SEQ ID NO: 343 and the heavy chain variable domain comprises the amino acid sequence of SEQ ID NO:530; (m) the light chain variable domain comprises the amino acid sequence of SEQ ID NO: 843 and the heavy chain variable domain comprises the amino acid sequence of SEQ ID NO:845; (n) the light chain variable domain comprises the amino acid sequence of SEQ ID NO: 844 and the heavy chain variable domain comprises the amino acid sequence of SEQ ID NO:846; (o) the light chain variable domain comprises the amino acid sequence of SEQ ID NO:844 and the heavy chain variable domain comprises the amino acid sequence of SEQ ID NO:847; (p) the light chain variable domain comprises the amino acid sequence of SEQ ID NO: 219 and the heavy chain variable domain comprises the amino acid sequence of SEQ ID NO:399; (q) the light chain variable domain comprises the amino acid sequence of SEQ ID NO: 230 and the heavy chain variable domain comprises the amino acid sequence of SEQ ID NO:409; (r) the light chain variable domain comprises the amino acid sequence of SEQ ID NO: 252 and the heavy chain variable domain comprises the amino acid sequence of SEQ ID NO:419; (s) the light chain variable domain comprises the amino acid sequence of SEQ ID NO: 241 and the heavy chain variable domain comprises the amino acid sequence of SEQ ID NO:429; (t) the light chain variable domain comprises the amino acid sequence of SEQ ID NO: 849 and the heavy chain variable domain comprises the amino acid sequence of SEQ ID NO:429; (u) the light chain variable domain comprises the amino acid sequence of SEQ ID NO: 263 and the heavy chain variable domain comprises the amino acid sequence of SEQ ID NO:439; (v) the light chain variable domain comprises the amino acid sequence of SEQ ID NO: 274 and the heavy chain variable domain comprises the amino acid sequence of SEQ ID NO:449; (w) the light chain variable domain comprises the amino acid sequence of SEQ ID NO:285 and the heavy chain variable domain comprises the amino acid sequence of SEQ ID NO:459; (x) the light chain variable domain comprises the amino acid sequence of SEQ ID NO:286 and the heavy chain variable domain comprises the amino acid sequence of SEQ ID NO:460; (y) the light chain variable domain comprises the amino acid sequence of SEQ ID NO: 287 and the heavy chain variable domain comprises the amino acid sequence of SEQ ID NO:461; (z) the light chain variable domain comprises the amino acid sequence of SEQ ID NO: 298 and the heavy chain variable domain comprises the amino acid sequence of SEQ ID NO:429; (aa) the light chain variable domain comprises the amino acid sequence of SEQ ID NO:299 and the heavy chain variable domain comprises the amino acid sequence of SEQ ID NO:471; (bb) the light chain variable domain comprises the amino acid sequence of SEQ ID NO: 310 and the heavy chain variable domain comprises the amino acid sequence of SEQ ID NO:461; (cc) the light chain variable domain comprises the amino acid sequence of SEQ ID NO: 679 and the heavy chain variable domain comprises the amino acid sequence of SEQ ID NO:481; (dd) the light chain variable domain comprises the amino acid sequence of SEQ ID NO: 311 and the heavy chain variable domain comprises the amino acid sequence of SEQ ID NO:491; (ee) the light chain variable domain comprises the amino acid sequence of SEQ ID NO: 322 and the heavy chain variable domain comprises the amino acid sequence of SEQ ID NO:511; (ff) the light chain variable domain comprises the amino acid sequence of SEQ ID NO: 344 and the heavy chain variable domain comprises the amino acid sequence of SEQ ID NO:531; (gg) the light chain variable domain comprises the amino acid sequence of SEQ ID NO: 355 and the heavy chain variable domain comprises the amino acid sequence of SEQ ID NO:635; (hh) the light chain variable domain comprises the amino acid sequence of SEQ ID NO: 365 and the heavy chain variable domain comprises the amino acid sequence of SEQ ID NO:541; (ii) the light chain variable domain comprises the amino acid sequence of SEQ ID NO: 376 and the heavy chain variable domain comprises the amino acid sequence of SEQ ID NO:551; (jj) the light chain variable domain comprises the amino acid sequence of SEQ ID NO: 387 and the heavy chain variable domain comprises the amino acid sequence of SEQ ID NO:561; (kk) the light chain variable domain comprises the amino acid sequence of SEQ ID NO: 398 and the heavy chain variable domain comprises the amino acid sequence of SEQ ID NO:571; (11) the light chain variable domain comprises the amino acid sequence of SEQ ID NO: 724 and the heavy chain variable domain comprises the amino acid sequence of SEQ ID NO:731; (mm) the light chain variable domain comprises the amino acid sequence of SEQ ID NO: 809 and the heavy chain variable domain comprises the amino acid sequence of SEQ ID NO:731; (nn) the light chain variable domain comprises the amino acid sequence of SEQ ID NO: 725 and the heavy chain variable domain comprises the amino acid sequence of SEQ ID NO:732; (oo) the light chain variable domain comprises the amino acid sequence of SEQ ID NO: 726 and the heavy chain variable domain comprises the amino acid sequence of SEQ ID NO:731; (pp) the light chain variable domain comprises the amino acid sequence of SEQ ID NO: 726 and the heavy chain variable domain comprises the amino acid sequence of SEQ ID NO:817; (qq) the light chain variable domain comprises the amino acid sequence of SEQ ID NO: 727 and the heavy chain variable domain comprises the amino acid sequence of SEQ ID NO:731; (rr) the light chain variable domain comprises the amino acid sequence of SEQ ID NO: 728 and the heavy chain variable domain comprises the amino acid sequence of SEQ ID NO:733; (ss) the light chain variable domain comprises the amino acid sequence of SEQ ID NO:810 and the heavy chain variable domain comprises the amino acid sequence of SEQ ID NO:818; (tt) the light chain variable domain comprises the amino acid sequence of SEQ ID NO:811 and the heavy chain variable domain comprises the amino acid sequence of SEQ ID NO:733; (uu) the light chain variable domain comprises the amino acid sequence of SEQ ID NO:729 and the heavy chain variable domain comprises the amino acid sequence of SEQ ID NO:731; (vv) the light chain variable domain comprises the amino acid sequence of SEQ ID NO:812 and the heavy chain variable domain comprises the amino acid sequence of SEQ ID NO:819; (ww) the light chain variable domain comprises the amino acid sequence of SEQ ID NO:729 and the heavy chain variable domain comprises the amino acid sequence of SEQ ID NO:820; (xx) the light chain variable domain comprises the amino acid sequence of SEQ ID NO: 730 and the heavy chain variable domain comprises the amino acid sequence of SEQ ID NO:731; (yy) the light chain variable domain comprises the amino acid sequence of SEQ ID NO:813 and the heavy chain variable domain comprises the amino acid sequence of SEQ ID NO:731; (zz) the light chain variable domain comprises the amino acid sequence of SEQ ID NO:814 and the heavy chain variable domain comprises the amino acid sequence of SEQ ID NO:822; (aaa) the light chain variable domain comprises the amino acid sequence of SEQ ID NO:815 and the heavy chain variable domain comprises the amino acid sequence of SEQ ID NO:824; or (bbb) the light chain variable domain comprises the amino acid sequence of SEQ ID NO:816 and the heavy chain variable domain comprises the amino acid sequence of SEQ ID NO:825.

Any of the antibodies of the present disclosure may be produced by a cell line. In some embodiments, the cell line may be a mammalian cell line. In certain embodiments, the cell line may be a hybridoma cell line. In other embodiments, the cell line may be a yeast cell line. Any cell line known in the art suitable for antibody production may be used to produce an antibody of the present disclosure. Exemplary cell lines for antibody production are described throughout the present disclosure.

In some embodiments, the anti-TREM2 antibody is an anti-TREM2 monoclonal antibody selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2, and humanized variants thereof. In certain embodiments, the anti-TREM2 antibody is an agonist antibody. In certain embodiments, the anti-TREM2 antibody is an inert antibody. In certain embodiments, the anti-TREM2 antibody is an antagonist antibody.

In some embodiments, the anti-TREM2 antibody is anti-TREM2 monoclonal antibody 7E5. In some embodiments, the anti-TREM2 antibody is an isolated antibody that binds essentially the same TREM2 epitope as 7E5. In some embodiments, the anti-TREM2 antibody is an isolated antibody comprising the HVR-H1, HVR-H2, and HVR-H3 of the heavy chain variable domain of monoclonal antibody 7E5. In some embodiments, the anti-TREM2 antibody is an isolated antibody comprising the HVR-L1, HVR-L2, and HVR-L3 of the light chain variable domain of monoclonal antibody 7E5. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 7E5.

In some embodiments, the anti-TREM2 antibody is anti-TREM2 monoclonal antibody 9F5. In some embodiments, the anti-TREM2 antibody is an isolated antibody which binds essentially the same TREM2 epitope as 9F5. In some embodiments, the anti-TREM2 antibody is an isolated antibody comprising the HVR-H1, HVR-H2, and HVR-H3 of the heavy chain variable domain of monoclonal antibody 9F5. In some embodiments, the anti-TREM2 antibody is an isolated antibody comprising the HVR-L1, HVR-L2, and HVR-L3 of the light chain variable domain of monoclonal antibody 9F5. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 9F5.

In some embodiments, the anti-TREM2 antibody is anti-TREM2 monoclonal antibody 3A7. In some embodiments, the anti-TREM2 antibody is an isolated antibody which binds essentially the same TREM2 epitope as 3A7. In some embodiments, the anti-TREM2 antibody is an isolated antibody comprising the HVR-H1, HVR-H2, and HVR-H3 of the heavy chain variable domain of monoclonal antibody 3A7. In some embodiments, the anti-TREM2 antibody is an isolated antibody comprising the HVR-L1, HVR-L2, and HVR-L3 of the light chain variable domain of monoclonal antibody 3A7. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 3A7.

In some embodiments, the anti-TREM2 antibody is anti-TREM2 monoclonal antibody 4D11. In some embodiments, the anti-TREM2 antibody is an isolated antibody, which binds essentially the same TREM2 epitope as 4D11. In some embodiments, the anti-TREM2 antibody is an isolated antibody comprising the HVR-H1, HVR-H2, and HVR-H3 of the heavy chain variable domain of monoclonal antibody 4D11. In some embodiments, the anti-TREM2 antibody is an isolated antibody comprising the HVR-L1, HVR-L2, and HVR-L3 of the light chain variable domain of monoclonal antibody 4D11. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 4D11.

In some embodiments, the anti-TREM2 antibody is anti-TREM2 monoclonal antibody 12F9. In some embodiments, the anti-TREM2 antibody is an isolated antibody which binds essentially the same TREM2 epitope as 12F9. In some embodiments, the anti-TREM2 antibody is an isolated antibody comprising the HVR-H1, HVR-H2, and HVR-H3 of the heavy chain variable domain of monoclonal antibody 12F9. In some embodiments, the anti-TREM2 antibody is an isolated antibody comprising the HVR-L1, HVR-L2, and HVR-L3 of the light chain variable domain of monoclonal antibody 12F9. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 12F9.

In some embodiments, the anti-TREM2 antibody is anti-TREM2 monoclonal antibody 8F8. In some embodiments, the anti-TREM2 antibody is an isolated antibody which binds essentially the same TREM2 epitope as 8F8. In some embodiments, the anti-TREM2 antibody is an isolated antibody comprising the HVR-H1, HVR-H2, and HVR-H3 of the heavy chain variable domain of monoclonal antibody 8F8. In some embodiments, the anti-TREM2 antibody is an isolated antibody comprising the HVR-L1, HVR-L2, and HVR-L3 of the light chain variable domain of monoclonal antibody 8F8. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 8F8.

In some embodiments, the anti-TREM2 antibody is anti-TREM2 monoclonal antibody 1B4. In some embodiments, the anti-TREM2 antibody is an isolated antibody which binds essentially the same TREM2 epitope as 1B4. In some embodiments, the anti-TREM2 antibody is an isolated antibody comprising the HVR-H1, HVR-H2, and HVR-H3 of the heavy chain variable domain of monoclonal antibody 1B4. In some embodiments, the anti-TREM2 antibody is an isolated antibody comprising the HVR-L1, HVR-L2, and HVR-L3 of the light chain variable domain of monoclonal antibody 1B4v1. In some embodiments, the anti-TREM2 antibody is an isolated antibody comprising the HVR-L1, HVR-L2, and HVR-L3 of the light chain variable domain of monoclonal antibody 1B4v2. In some embodiments, the anti-TREM2 antibody is an isolated antibody comprising the HVR-H1, HVR-H2, and HVR-H3 of the heavy chain variable domain of monoclonal antibody 1B4, and the HVR-L1, HVR-L2, and HVR-L3 of the light chain variable domain of monoclonal antibody 1B4v1. In some embodiments, the anti-TREM2 antibody is an isolated antibody comprising the HVR-H1, HVR-H2, and HVR-H3 of the heavy chain variable domain of monoclonal antibody 1B4, and the HVR-L1, HVR-L2, and HVR-L3 of the light chain variable domain of monoclonal antibody 1B4v2.

In some embodiments, the anti-TREM2 antibody is anti-TREM2 monoclonal antibody 6H2. In some embodiments, the anti-TREM2 antibody is an isolated antibody which binds essentially the same TREM2 epitope as 6H2. In some embodiments, the anti-TREM2 antibody is an isolated antibody comprising the HVR-H1, HVR-H2, and HVR-H3 of the heavy chain variable domain of monoclonal antibody 6H2. In some embodiments, the anti-TREM2 antibody is an isolated antibody comprising the HVR-L1, HVR-L2, and HVR-L3 of the light chain variable domain of monoclonal antibody 6H2. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 6H2.

In some embodiments, the anti-TREM2 antibody is anti-TREM2 monoclonal antibody 7B11. In some embodiments, the anti-TREM2 antibody is an isolated antibody which binds essentially the same TREM2 epitope as 7B11. In some embodiments, the anti-TREM2 antibody is an isolated antibody comprising the HVR-H1, HVR-H2, and HVR-H3 of the heavy chain variable domain of monoclonal antibody 7B11v1. In some embodiments, the anti-TREM2 antibody is an isolated antibody comprising the HVR-H1, HVR-H2, and HVR-H3 of the heavy chain variable domain of monoclonal antibody 7B11v2. In some embodiments, the anti-TREM2 antibody is an isolated antibody comprising the HVR-L1, HVR-L2, and HVR-L3 of the light chain variable domain of monoclonal antibody 7B11. In some embodiments, the anti-TREM2 antibody is an isolated antibody comprising the HVR-H1, HVR-H2, and HVR-H3 of the heavy chain variable domain of monoclonal antibody 7B11v1, and the HVR-L1, HVR-L2, and HVR-L3 of the light chain variable domain of monoclonal antibody 7B11. In some embodiments, the anti-TREM2 antibody is an isolated antibody comprising the HVR-H1, HVR-H2, and HVR-H3 of the heavy chain variable domain of monoclonal antibody 7B11v2, and the HVR-L1, HVR-L2, and HVR-L3 of the light chain variable domain of monoclonal antibody 7B11.

In some embodiments, the anti-TREM2 antibody is anti-TREM2 monoclonal antibody 18D8. In some embodiments, the anti-TREM2 antibody is an isolated antibody which binds essentially the same TREM2 epitope as 18D8. In some embodiments, the anti-TREM2 antibody is an isolated antibody comprising the HVR-H1, HVR-H2, and HVR-H3 of the heavy chain variable domain of monoclonal antibody 18D8. In some embodiments, the anti-TREM2 antibody is an isolated antibody comprising the HVR-L1, HVR-L2, and HVR-L3 of the light chain variable domain of monoclonal antibody 18D8. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 18D8.

In some embodiments, the anti-TREM2 antibody is anti-TREM2 monoclonal antibody 18E4v1. In some embodiments, the anti-TREM2 antibody is an isolated antibody which binds essentially the same TREM2 epitope as 18E4v1. In some embodiments, the anti-TREM2 antibody is an isolated antibody comprising the HVR-H1, HVR-H2, and HVR-H3 of the heavy chain variable domain of monoclonal antibody 18E4v1. In some embodiments, the anti-TREM2 antibody is an isolated antibody comprising the HVR-L1, HVR-L2, and HVR-L3 of the light chain variable domain of monoclonal antibody 18E4v1. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 18E4v1.

In some embodiments, the anti-TREM2 antibody is anti-TREM2 monoclonal antibody 18E4v2. In some embodiments, the anti-TREM2 antibody is an isolated antibody which binds essentially the same TREM2 epitope as 18E4v2. In some embodiments, the anti-TREM2 antibody is an isolated antibody comprising the HVR-H1, HVR-H2, and HVR-H3 of the heavy chain variable domain of monoclonal antibody 18E4v2. In some embodiments, the anti-TREM2 antibody is an isolated antibody comprising the HVR-L1, HVR-L2, and HVR-L3 of the light chain variable domain of monoclonal antibody 18E4v2. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 18E4v2.

In some embodiments, the anti-TREM2 antibody is anti-TREM2 monoclonal antibody 29F6v1. In some embodiments, the anti-TREM2 antibody is an isolated antibody which binds essentially the same TREM2 epitope as 29F6v1. In some embodiments, the anti-TREM2 antibody is an isolated antibody comprising the HVR-H1, HVR-H2, and HVR-H3 of the heavy chain variable domain of monoclonal antibody 29F6v1. In some embodiments, the anti-TREM2 antibody is an isolated antibody comprising the HVR-L1, HVR-L2, and HVR-L3 of the light chain variable domain of monoclonal antibody 29F6v1. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 29F6v1.

In some embodiments, the anti-TREM2 antibody is anti-TREM2 monoclonal antibody 29F6v2. In some embodiments, the anti-TREM2 antibody is an isolated antibody which binds essentially the same TREM2 epitope as 29F6v2. In some embodiments, the anti-TREM2 antibody is an isolated antibody comprising the HVR-H1, HVR-H2, and HVR-H3 of the heavy chain variable domain of monoclonal antibody 29F6v2. In some embodiments, the anti-TREM2 antibody is an isolated antibody comprising the HVR-L1, HVR-L2, and HVR-L3 of the light chain variable domain of monoclonal antibody 29F6v2. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 29F6v2.

In some embodiments, the anti-TREM2 antibody is anti-TREM2 monoclonal antibody 40D5. In some embodiments, the anti-TREM2 antibody is an isolated antibody which binds essentially the same TREM2 epitope as 40D5. In some embodiments, the anti-TREM2 antibody is an isolated antibody comprising the HVR-H1, HVR-H2, and HVR-H3 of the heavy chain variable domain of monoclonal antibody 40D5v1. In some embodiments, the anti-TREM2 antibody is an isolated antibody comprising the HVR-H1, HVR-H2, and HVR-H3 of the heavy chain variable domain of monoclonal antibody 40D5v2. In some embodiments, the anti-TREM2 antibody is an isolated antibody comprising the HVR-L1, HVR-L2, and HVR-L3 of the light chain variable domain of monoclonal antibody 40D5. In some embodiments, the anti-TREM2 antibody is an isolated antibody comprising the HVR-H1, HVR-H2, and HVR-H3 of the heavy chain variable domain of monoclonal antibody 40D5v1, and the HVR-L1, HVR-L2, and HVR-L3 of the light chain variable domain of monoclonal antibody 40D5. In some embodiments, the anti-TREM2 antibody is an isolated antibody comprising the HVR-H1, HVR-H2, and HVR-H3 of the heavy chain variable domain of monoclonal antibody 40D5v2, and the HVR-L1, HVR-L2, and HVR-L3 of the light chain variable domain of monoclonal antibody 40D5.

In some embodiments, the anti-TREM2 antibody is anti-TREM2 monoclonal antibody 43B9. In some embodiments, the anti-TREM2 antibody is an isolated antibody which binds essentially the same TREM2 epitope as 43B9. In some embodiments, the anti-TREM2 antibody is an isolated antibody comprising the HVR-H1, HVR-H2, and HVR-H3 of the heavy chain variable domain of monoclonal antibody 43B9. In some embodiments, the anti-TREM2 antibody is an isolated antibody comprising the HVR-L1, HVR-L2, and HVR-L3 of the light chain variable domain of monoclonal antibody 43B9. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 43B9.

In some embodiments, the anti-TREM2 antibody is anti-TREM2 monoclonal antibody 44A8. In some embodiments, the anti-TREM2 antibody is an isolated antibody which binds essentially the same TREM2 epitope as 44A8. In some embodiments, the anti-TREM2 antibody is an isolated antibody comprising the HVR-H1, HVR-H2, and HVR-H3 of the heavy chain variable domain of monoclonal antibody 44A8. In some embodiments, the anti-TREM2 antibody is an isolated antibody comprising the HVR-L1, HVR-L2, and HVR-L3 of the light chain variable domain of monoclonal antibody 44A8v1. In some embodiments, the anti-TREM2 antibody is an isolated antibody comprising the HVR-L1, HVR-L2, and HVR-L3 of the light chain variable domain of monoclonal antibody 44A8v2. In some embodiments, the anti-TREM2 antibody is an isolated antibody comprising the HVR-H1, HVR-H2, and HVR-H3 of the heavy chain variable domain of monoclonal antibody 44A8, and the HVR-L1, HVR-L2, and HVR-L3 of the light chain variable domain of monoclonal antibody 44A8v1. In some embodiments, the anti-TREM2 antibody is an isolated antibody comprising the HVR-H1, HVR-H2, and HVR-H3 of the heavy chain variable domain of monoclonal antibody 44A8, and the HVR-L1, HVR-L2, and HVR-L3 of the light chain variable domain of monoclonal antibody 44A8v2.

In some embodiments, the anti-TREM2 antibody is anti-TREM2 monoclonal antibody 44B4v1. In some embodiments, the anti-TREM2 antibody is an isolated antibody which binds essentially the same TREM2 epitope as 44B4v1. In some embodiments, the anti-TREM2 antibody is an isolated antibody comprising the HVR-H1, HVR-H2, and HVR-H3 of the heavy chain variable domain of monoclonal antibody 44B4v1. In some embodiments, the anti-TREM2 antibody is an isolated antibody comprising the HVR-L1, HVR-L2, and HVR-L3 of the light chain variable domain of monoclonal antibody 44B4v1. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 44B4v1.

In some embodiments, the anti-TREM2 antibody is anti-TREM2 monoclonal antibody 44B4v2. In some embodiments, the anti-TREM2 antibody is an isolated antibody which binds essentially the same TREM2 epitope as 44B4v2. In some embodiments, the anti-TREM2 antibody is an isolated antibody comprising the HVR-H1, HVR-H2, and HVR-H3 of the heavy chain variable domain of monoclonal antibody 44B4v2. In some embodiments, the anti-TREM2 antibody is an isolated antibody comprising the HVR-L1, HVR-L2, and HVR-L3 of the light chain variable domain of monoclonal antibody 44B4v2. In some embodiments, the anti-TREM2 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 44B4v2.

In some embodiments, anti-TREM2 antibodies of the present disclosure do not compete with one or more TREM2 ligands for binding to TREM2. In some embodiments, anti-TREM2 antibodies of the present disclosure are capable of binding TREM2 without blocking simultaneous binding of one or moreTREM2 ligands to TREM2. In some embodiments anti-TREM2 antibodies of the present disclosure are capable of additive and/or synergistic functional interactions with one or more TREM2 ligands. In some embodiments, anti-TREM2 antibodies of the present disclosure increase the maximal activity of TREM2 exposed to saturating concentrations of one or more TREM2 ligands. In some embodiments, anti-TREM2 antibodies of the present disclosure increase the activity of TREM2 obtained at any concentration of one or more TREM2 ligands.

Anti-TREM2 Antibody Binding Affinity

The dissociation constants (K_{D}) of anti-TREM2 antibodies for human TREM2 and mouse TREM2 may be less than 15 nM, less than 14.5 nM, less than 14 nM, less than 13.5 nM, less than 13 nM, less than 12.9 nM, less than 12.8 nM, less than 12.7 nM, less than 12.6 nM, less than 12.5 nM, less than 12.4 nM, less than 12.3 nM, less than 12.2 nM, less than 12.1 nM, less than 12 nM, less than 11.5 nM, less than 11 nM, less than 10.9 nM, less than 10.8 nM, less than 10.7 nM, less than 10.6 nM, less than 10.5 nM, less than 10.4 nM, less than 10.3 nM, less than 10.2 nM, less than 10.1 nM, less than 10 nM, less than 9.5 nM, less than 9 nM, less than 8.5 nM, less than 8 nM, less than 7.5 nM, less than 7 nM, less than 6.9 nM, less than 6.8 nM, less than 6.7 nM, less than 6.6 nM, less than 6.5 nM, less than 6.4 nM, less than 6.3 nM, less than 6.2 nM, less than 6.1 nM, less than 6 nM, less than 5.5 nM, less than 5 nM, less than 4.5 nM, less than 4 nM, less than 3.5 nM, less than 3.4 nM, less than 3.3 nM, less than 3.2 nM, less than 3.1 nM, less than 3 nM, less than 2.9 nM, less than 2.8 nM, less than 2.7 nM, less than 2.6 nM, less than 2.5 nM, less than 2.4 nM, less than 2.3 nM, less than 2.2 nM, less than 2.1 nM, less than 2 nM, less than 1.9 nM, less than 1.8 nM, less than 1.7 nM, less than 1.6 nM, less than 1.5 nM, less than 1.4 nM, less than 1.3 nM, less than 1.2 nM, less than 1.1 nM, less than 1 nM, less than 0.95 nM, or less than 0.9 nM. In some embodiments, dissociation constants range from about 12.8 nM to about 1.2 nM, or less than 1.2 nM. In some embodiments, dissociation constants of anti-TREM2 antibodies for human TREM2 range from about 12.8 nM to about 2.9 nM, or less than 2.9 nM. In some embodiments, dissociation constants of anti-TREM2 antibodies for mouse TREM2 range from about 10.4 nM to about 1.2 nM, or less than 1.2 nM.

In some embodiments, anti-TREM2 antibodies of the present disclosure increase memory and/or reduce cognitive deficit when administered to an individual. In some embodiments, anti-TREM2 antibodies of the present disclosure do not inhibit the growth of one or more innate immune cells. In some embodiments, anti-TREM2 antibodies of the present disclosure bind to one or more primary immune cells with a K_{D} of less than 50 nM, less than 45 nM, less than 40 nM, less than 35 nM, less than 30 nM, less than 25 nM, less than 20 nM, less than 15 nM, less than 10 nM, less than 9 nM, less than 8 nM, less than 7 nM, less than 6 nM, less than 5 nM, less than 4 nM, less than 3 nM, less than 2 nM, or less than 1 nM. In some embodiments, the dissociation constant (K_{D}) is determined at a temperature of approximately 4° C. In some embodiments, the K_{D} is determined using a monovalent antibody (e.g., a Fab) or a full-length antibody in a monovalent form. Methods for the preparation and selection of antibodies that interact and/or bind with specificity to TREM2 are described herein. (e.g., see Example 1).

Dissociation constants may be determined through any analytical technique, including any biochemical or biophysical technique such as ELISA, surface plasmon resonance (SPR), bio-layer interferometry (see, e.g., Octet System by ForteBio), isothermal titration calorimetry (ITC), differential scanning calorimetry (DSC), circular dichroism (CD), stopped-flow analysis, and colorimetric or fluorescent protein melting analyses. In some embodiments, the dissociation constant (K_{D}) for TREM2 is determined at a temperature of approximately 4° C. In some embodiments, the K_{D} is determined using a monovalent antibody (e.g., a Fab) or a full-length antibody. In some embodiments, the K_{D} is determined using a full-length antibody in a monovalent form. Utilizing, for example, any assay described herein (see, e.g., Example 1).

Additional anti-TREM2 antibodies, e.g., antibodies that specifically bind to a TREM2 protein of the present disclosure, may be identified, screened, and/or characterized for their physical/chemical properties and/or biological activities by various assays known in the art.

Bispecific Antibodies

Certain aspects of the present disclosure relate to bispecific antibodies that bind to a TREM2 protein of the present disclosure and a second antigen. Methods of generating bispecific antibodies are well known in the art and described herein. In some embodiments, bispecific antibodies of the present disclosure bind to one or more amino acid residues of human TREM2 (SEQ ID NO: 1), or amino acid residues on a TREM2 protein corresponding to amino acid residues of SEQ ID NO: 1. In other embodiments, bispecific antibodies of the present disclosure also bind to one or more amino acid residues of human DAP12 (SEQ ID NO: 887), or amino acid residues on a DAP12 protein corresponding to amino acid residues of SEQ ID NO: 887.

In some embodiments, bispecific antibodies of the present disclosure recognize a first antigen and a second antigen. In some embodiments, the first antigen is human TREM2 or a naturally occurring variant thereof, or human DAP12 or a naturally occurring variant thereof. In some embodiments, the second antigen is a) an antigen facilitating transport across the blood-brain-barrier; (b) an antigen facilitating transport across the blood-brain-barrier selected from transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, and ANG1005; (c) a disease-causing protein selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; and (d) ligands and/or proteins expressed on immune cells, wherein the ligands and/or proteins selected from CD40, OX40, ICOS, CD28, CD137/4-1BB, CD27, GITR, PD-L1, CTLA-4, PD-L2, PD-1, B7-H3, B7-H4, HVEM, BTLA, KIR, GALS, TIM3, A2AR, LAG-3, and phosphatidylserine; and (e) a protein, lipid, polysaccharide, or glycolipid expressed on one or more tumor cells and any combination thereof.

Antibody Fragments

Certain aspects of the present disclosure relate to antibody fragments that bind to one or more of human TREM2, a naturally occurring variant of human TREM2, and a disease variant of human TREM2. In some embodiments, the antibody fragment is an Fab, Fab′, Fab′-SH, F(ab′)2, Fv or scFv fragment. In some embodiments, the antibody fragment is used in combination with one or more antibodies that specifically bind a disease-causing protein selected from: a) an antigen facilitating transport across the blood-brain-barrier; (b) an antigen facilitating transport across the blood-brain-barrier selected from transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, and ANG1005; (c) a disease-causing protein selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; and (d) ligands and/or proteins expressed on immune cells, wherein the ligands and/or proteins selected from CD40, OX40, ICOS, CD28, CD137/4-1BB, CD27, GITR, PD-L1, CTLA-4, PD-L2, PD-1, B7-H3, B7-H4, HVEM, BTLA, KIR, GALS, TIM3, A2AR, LAG-3, and phosphatidylserine; and (e) a protein, lipid, polysaccharide, or glycolipid expressed on one or more tumor cells, and any combination thereof.

Antibody Frameworks

Any of the antibodies described herein further include a framework. In some embodiments, the framework is a human immunoglobulin framework. For example, in some embodiments, an antibody (e.g., an anti-TREM2 antibody) comprises HVRs as in any of the above embodiments and further comprises an acceptor human framework, e.g., a human immunoglobulin framework or a human consensus framework. Human immunoglobulin frameworks may be part of the human antibody, or a non-human antibody may be humanized by replacing one or more endogenous frameworks with human framework region(s). Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In some embodiments, an antibody comprises a light chain variable region comprising an HVR-L1, an HVR-L2, and an HVR-L3 of the present disclosure and one, two, three or four of the light chain framework regions as shown in Table 4A. In some embodiments, an antibody comprises a heavy chain variable region comprising an HVR-H1, an HVR-H2, and an HVR-H3 of the present disclosure and one, two, three or four of the heavy chain framework regions as shown in Table 4B. In some embodiments, an antibody comprises a light chain variable region comprising an HVR-L1, an HVR-L2, and an HVR-L3 of the present disclosure and one, two, three or four of the light chain framework regions as shown in Table 4A and further comprises a heavy chain variable region comprising an HVR-H1, an HVR-H2, and an HVR-H3 of the present disclosure and one, two, three or four of the heavy chain framework regions as shown in Table 4B.

PI3K Activation

In some embodiments, the anti-TREM2 antibodies of the present disclosure may induce PI3K activation after binding to a TREM2 protein expressed in a cell.

PI3Ks are a family of related intracellular signal transducer kinases capable of phosphorylating the 3-position hydroxyl group of the inositol ring of phosphatidylinositol (PtdIns). The PI3K family is divided into three different classes (Class I, Class II, and Class III) based on primary structure, regulation, and in vitro lipid substrate specificity.

Activated PI3K produces various 3-phosphorylated phosphoinositides, including without limitation, PtdIns3P, Ptdlns(3,4)P2, Ptdlns(3,5)P2, and Ptdlns(3,4,5)P3. These 3-phosphorylated phosphoinositides function in a mechanism by which signaling proteins are recruited to various cellular membranes. These signaling proteins contain phosphoinositide-binding domains, including without limitation, PX domains, pleckstrin homology domains (PH domains), and FYVE domains. Any method known in the art for determining PI3K activation may be used.

In some embodiments, anti-TREM2 antibodies of the present disclosure may be beneficial for preventing, lowering the risk of, or treating conditions and/or diseases associated with decreased levels of PI3K activity, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that do not, inhibits interaction between TREM2 and one or more TREM2 ligands, and/or enhance one or more activities of at least one TREM2 ligand. Other aspects of the present disclosure relate to an agent that do not, inhibits interaction between TREM2 and one or more TREM2 ligands, and/or enhance one or more activities of at least one TREM2 ligand, for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

Modulated Expression of Anti-Inflammatory Mediators

In some embodiments, the anti-TREM2 antibodies of the present disclosure modulate (e.g., increase or decrease) anti-inflammatory mediators in the brain after binding to a TREM2 protein expressed on a cell surface. The anti-TREM2 antibodies of the present disclosure modulate the expression of cytokines (e.g., anti-inflammatory mediators) and/or modulate the expression of pro-inflammatory mediators after binding to a TREM2 protein expressed in a cell. Once the cells are dying due to deficiency in TREM2 signaling they induce a pro inflammatory response.

Inflammation is part of a complex biological response of vascular tissues to harmful stimuli, such as pathogens, damaged cells, and irritants. The classical signs of acute inflammation are pain, heat, redness, swelling, and loss of function. Inflammation is a protective attempt by an organism to remove the injurious stimuli and to initiate the healing process. Inflammation can be classified as either acute inflammation or chronic inflammation. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Chronic inflammation is prolonged inflammation that leads to a progressive shift in the type of cells present at the site of inflammation and is characterized by simultaneous destruction and healing of the tissue from the inflammatory process.

As used herein, anti-inflammatory mediators are proteins involved either directly or indirectly (e.g., by way of an anti-inflammatory signaling pathway) in a mechanism that reduces, inhibits, or inactivates an inflammatory response. Any method known in the art for identifying and characterizing anti-inflammatory mediators may be used. Examples of anti-inflammatory mediators include, without limitation, cytokines, such as IL-4, IL-10 TGF-β, IL-13, IL-35 IL-16, IFN-alpha, IL-1Ra, VEGF, G-CSF, YM, AXL, FLT1 and soluble receptors for TNF or IL-6

In some embodiments, the anti-TREM2 antibodies of the present disclosure may modulate expression of cytokines, such as IL-12p70, IL-6, and IL-10. In certain embodiments, modulated expression of the cytokines occurs in macrophages, dendritic cells, monocytes, osteoclasts, Langerhans cells of skin, Kupffer cells, and/or microglial cells. Modulated expression may include, without limitation, modulated gene expression, modulated transcriptional expression, or modulated protein expression. Any method known in the art for determining gene, transcript (e.g., mRNA), and/or protein expression may be used. For example, Northern blot analysis may be used to determine cytokine gene expression levels, RT-PCR may be used to determine the level of cytokine transcription, and Western blot analysis may be used to determine cytokine protein levels.

As used herein, a cytokine may have modulated (e.g., increased or decreased) expression if its expression in one or more cells of a subject treated with an anti-TREM2 antibody of the present disclosure is modulated as compared to the expression of the same cytokine expressed in one or more cells of a corresponding subject that is not treated with the anti-TREM2 antibody. In some embodiments, an anti-TREM2 antibody of the present disclosure may modulate cytokine expression in one or more cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to cytokine expression in one or more cells of a corresponding subject that is not treated with the anti-TREM2 antibody. In other embodiments, an anti-TREM2 antibody of the present disclosure modulate cytokine expression in one or more cells of a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to cytokine expression in one or more cells of a corresponding subject that is not treated with the anti-TREM2 antibody.

In some embodiments, anti-TREM2 antibodies of the present disclosure may be useful for preventing, lowering the risk of, or treating conditions and/or diseases associated with abnormal levels of one or more anti-inflammatory mediators, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibits interaction between TREM2 and one or more TREM2 ligands, and/or enhance one or more activities of at least one TREM2 ligand. Other aspects of the present disclosure relate to an agent that does not inhibits interaction between TREM2 and one or more TREM2 ligands, and/or enhance one or more activities of at least one TREM2 ligand, for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

Modulated Expression of Pro-Inflammatory Mediators

In some embodiments, the anti-TREM2 antibodies of the present disclosure may modulate (e.g., increase or decrease) the expression of pro-inflammatory mediators after binding to a TREM2 protein expressed in a cell.

As used herein, pro-inflammatory mediators are proteins involved either directly or indirectly (e.g., by way of pro-inflammatory signaling pathways) in a mechanism that induces, activates, promotes, or otherwise increases an inflammatory response. Any method known in the art for identifying and characterizing pro-inflammatory mediators may be used. Examples of pro-inflammatory mediators include, without limitation, cytokines such as IFN-β, IL-1α, IL-1β, CD86, TNF-α, IL-6, IL-8, CRP, MCP-1/CCL2, CCL3, CCL4, CCL5, CCR2, CXCL-10, Gata3, IL-20 family members, IL-33, LIF, IFN-gamma, OSM, CNTF, CSF1, OPN, CD11c, GM-CSF, IL-11, IL-12, IL-17, IL-18, and IL-23.

In some embodiments, the anti-TREM2 antibodies of the present disclosure may modulate functional expression and/or secretion of pro-inflammatory mediators, such as IFN-β, IL-la, IL-1β, CD86, TNF-α, IL-6, IL-8, CRP, MCP-1/CCL2, CCL3, CCL4, CCL5, CCR2, CXCL-10, Gata3, IL-20 family members, IL-33, LIF, IFN-gamma, OSM, CNTF, CSF1, OPN, CD11c, GM-CSF, IL-11, IL-12, IL-17, IL-18, and IL-23. In certain embodiments, modulated expression of the pro-inflammatory mediators occurs in macrophages, dendritic cells, monocytes, osteoclasts, Langerhans cells of skin, Kupffer cells, and/or microglial cells. Modulated expression may include, without limitation, modulated gene expression, modulated transcriptional expression, or modulated protein expression. Any method known in the art for determining gene, transcript (e.g., mRNA), and/or protein expression may be used. For example, Northern blot analysis may be used to determine pro-inflammatory mediator gene expression levels, RT-PCR may be used to determine the level of pro-inflammatory mediator transcription, and Western blot analysis may be used to determine pro-inflammatory mediator protein levels.

In certain embodiments, pro-inflammatory mediators include inflammatory cytokines. Accordingly, in certain embodiments, the anti-TREM2 antibodies of the present disclosure may modulate secretion of one or more inflammatory cytokines. Examples of inflammatory cytokines whose secretion may be reduced by the anti-TREM2 antibodies of the present disclosure include, without limitation, IFN-β, IL-1α, IL-1β, CD86, TNF-α, IL-6, IL-8, CRP, MCP-1/CCL2, CCL3, CCL4, CCL5, CCR2, CXCL-10, Gata3, IL-20 family members, IL-33, LIF, IFN-gamma, OSM, CNTF, CSF1, OPN, CD11c, GM-CSF, IL-11, IL-12, IL-17, IL-18, and IL-23.

In certain embodiments, pro-inflammatory mediators include inflammatory receptors. Accordingly, in certain embodiments, the anti-TREM2 antibodies of the present disclosure may modulate expression of one or more inflammatory receptors. Examples of inflammatory receptors whose expression may be reduced by the anti-TREM2 antibodies of the present disclosure include, without limitation, CD86.

As used herein, a pro-inflammatory mediator may have modulated expression if its expression in one or more cells of a subject treated with an agonist anti-TREM2 antibody of the present disclosure is modulated (e.g., increased or decreased) as compared to the expression of the same pro-inflammatory mediator expressed in one or more cells of a corresponding subject that is not treated with the agonist anti-TREM2 antibody. In some embodiments, the agonist anti-TREM2 antibody of the present disclosure may modulate pro-inflammatory mediator expression in one or more cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to pro-inflammatory mediator expression in one or more cells of a corresponding subject that is not treated with the agonist anti-TREM2 antibody. In other embodiments, the agonist anti-TREM2 antibody may modulate pro-inflammatory mediator expression in one or more cells of a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to pro-inflammatory mediator expression in one or more cells of a corresponding subject that is not treated with the anti-TREM2 antibody.

In some embodiments, anti-TREM2 antibodies of the present disclosure may be useful for preventing, lowering the risk of, or treating conditions and/or diseases associated with abnormal levels of one or more pro-inflammatory mediators, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibit interaction between TREM2 and one or more TREM2 ligands, and/or enhance one or more activities of at least one TREM2 ligand. Other aspects of the present disclosure relate to an agent that does not inhibit interaction between TREM2 and one or more TREM2 ligands, and/or enhance one or more activities of at least one TREM2 ligand, for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

ERK Phosphorylation

In some embodiments, the anti-TREM2 antibodies of the present disclosure may induce extracellular signal-regulated kinase (ERK) phosphorylation after binding to a TREM2 protein expressed in a cell.

Extracellular-signal-regulated kinases (ERKs) are widely expressed protein kinase intracellular signaling kinases that are involved in, for example, the regulation of meiosis, mitosis, and postmitotic functions in differentiated cells. Various stimuli, such as growth factors, cytokines, virus infection, ligands for heterotrimeric G protein-coupled receptors, transforming agents, and carcinogens, activate ERK pathways. Phosphorylation of ERKs leads to the activation of their kinase activity.

In some embodiments, anti-TREM2 antibodies of the present disclosure may be beneficial for preventing, lowering the risk of, or treating conditions and/or diseases associated with decreased levels of ERK phosphorylation, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not, inhibit interaction between TREM2 and one or more TREM2 ligands, and/or enhance one or more activities of at least one TREM2 ligand. Other aspects of the present disclosure relate to an agent that does not inhibit interaction between TREM2 and one or more TREM2 ligands, and/or enhance one or more activities of at least one TREM2 ligand, for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

Syk Phosphorylation

In some embodiments, the anti-TREM2 antibodies of the present disclosure may induce spleen tyrosine kinase (Syk) phosphorylation after binding to a TREM2 protein expressed in a cell.

Spleen tyrosine kinase (Syk) is an intracellular signaling molecule that functions downstream of TREM2 by phosphorylating several substrates, thereby facilitating the formation of a signaling complex leading to cellular activation and inflammatory processes.

In some embodiments, anti-TREM2 antibodies of the present disclosure may be beneficial for preventing, lowering the risk of, or treating conditions and/or diseases associated with decreased levels of Syk phosphorylation, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibit interaction between TREM2 and one or more TREM2 ligands, and/or enhance one or more activities of at least one TREM2 ligand. Other aspects of the present disclosure relate to an agent that does not inhibit interaction between TREM2 and one or more TREM2 ligands, and/or enhance one or more activities of at least one TREM2 ligand, for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

TREM2 Autophosphorylation

In some embodiments, the anti-TREM2 a antibodies of the present disclosure may induce TREM2 autophosphorylation after binding to a TREM2 protein expressed in a cell.

In some embodiments, anti-TREM2 antibodies of the present disclosure may be beneficial for preventing, lowering the risk of, or treating conditions and/or diseases associated with decreased levels of TREM2 phosphorylation, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibit interaction between TREM2 and one or more TREM2 ligands, and/or enhance one or more activities of at least one TREM2 ligand. Other aspects of the present disclosure relate to an agent that does not inhibit interaction between TREM2 and one or more TREM2 ligands, and/or enhance one or more activities of at least one TREM2 ligand, for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

DAP12 Binding and Phosphorylation

In some embodiments, the anti-TREM2 antibodies of the present disclosure may induce binding of TREM2 to DAP12. In other embodiments, the anti-TREM2 antibodies of the present disclosure may induce DAP12 phosphorylation after binding to a TREM2 protein expressed in a cell. In other embodiments, TREM2-mediated DAP12 phosphorylation is induced by one or more SRC family tyrosine kinases. Examples of Src family tyrosine kinases include, without limitation, Src, Syk, Yes, Fyn, Fgr, Lck, Hck, Blk, Lyn, and Frk.

DAP12 is variously referred to as TYRO protein tyrosine kinase-binding protein, TYROBP, KARAP, and PLOSL. DAP12 is a transmembrane signaling protein that contains an immunoreceptor tyrosine-based activation motif (ITAM) in its cytoplasmic domain. In certain embodiments, the anti-TREM2 and/or anti-DAP12 antibody may induce DAP12 phosphorylation in its ITAM motif. Any method known in the art for determining protein phosphorylation, such as DAP12 phosphorylation, may be used.

In some embodiments, DAP12 is phosphorylated by SRC family kinases, resulting in the recruitment and activation of the Syk kinase, ZAP70 kinase, or both, to a DAP12/TREM2 complex. Thus, in certain embodiments, the anti-TREM2 antibodies of the present disclosure may recruit Syk, ZAP70, or both to a DAP12/TREM2 complex. Without wishing to be bound by theory, it is believed that anti-TREM2 a antibodies of the present disclosure are useful for preventing, lowering the risk of, or treating conditions and/or diseases associated with decreased levels of DAP12 activity, DAP12 phosphorylation, or recruitment of Syk, ZAP70, or both to a DAP12/TREM2 complex, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibit interaction between TREM2 and one or more TREM2 ligands, and/or enhance one or more activities of one or more TREM2 ligands, Other aspects of the present disclosure relate to an agent does not inhibit interaction between TREM2 and one or more TREM2 ligands, and/or enhance one or more activities of one or more TREM2 ligands, use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

Modulated Expression of C-C Chemokine Receptor 7

In some embodiments, the anti-TREM2 antibodies of the present disclosure may modulate (e.g., increase or decrease) expression of C-C chemokine receptor 7 (CCR7) after binding to a TREM2 protein expressed in a cell. Modulated expression may include, without limitation, modulation in gene expression, modulation in transcriptional expression, or modulation in protein expression. Any method known in the art for determining gene, transcript (e.g., mRNA), and/or protein expression may be used. For example, Northern blot analysis may be used to determine anti-inflammatory mediator gene expression levels, RT-PCR may be used to determine the level of anti-inflammatory mediator transcription, and Western blot analysis may be used to determine anti-inflammatory mediator protein levels.

C-C chemokine receptor 7 (CCR7) is a member of the G protein-coupled receptor family. CCR7 is expressed in various lymphoid tissues and can activate B-cells and T-cells. In some embodiments, CCR7 may modulate the migration of memory T-cells to secondary lymphoid organs, such as lymph nodes. In other embodiments, CCR7 may stimulate dendritic cell maturation. CCR7 is a receptor protein that can bind the chemokine (C-C motif) ligands CCL19/ELC and CCL21.

As used herein, CCR7 may have modulated (e.g., increased or decreased) expression if its expression in one or more cells of a subject treated with an anti-TREM2 antibody of the present disclosure is modulated as compared to the expression of CCR7 expressed in one or more cells of a corresponding subject that is not treated with the anti-TREM2 antibody. In some embodiments, an anti-TREM2 antibody of the present disclosure may modulate CCR7 expression in one or more cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to CCR7 expression in one or more cells of a corresponding subject that is not treated with the anti-TREM2 antibody. In other embodiments, an anti-TREM2 antibody of the present disclosure modulates CCR7 expression in one or more cells of a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to CCR7 expression in one or more cells of a corresponding subject that is not treated with the anti-TREM2 antibody.

In some embodiments, modulated expression of CCR7 occurs in macrophages, dendritic cells, and/or microglial cells. Increased expression of CCR7 may induce microglial cell chemotaxis toward cells expressing the chemokines CCL19 and CCL21. Accordingly, in certain embodiments, anti-TREM2 antibodies of the present disclosure may induce microglial cell chemotaxis toward CCL19 and CCL21 expressing cells.

In some embodiments, anti-TREM2 antibodies of the present disclosure may be useful for preventing, lowering the risk of, or treating conditions and/or diseases associated with abnormal levels of CCR7, including dementia, frontotemporal dementia, Alzheimer's disease, Nasu-Hakola disease, Parkinson's disease, Amyotrophic lateral sclerosis, Huntington's disease, and tauopathy disease.

Modulated Expression of Genes Induced by Inflammation

In some embodiments, the anti-TREM2 antibodies of the present disclosure may modulate (e.g., increase or decrease) expression of one or more genes whose expression is increased upon induction of inflammation after binding to a TREM2 protein expressed in a cell. Examples of such genes include, without limitation, Fabp3, Fabp5, and LDR. Modulated expression may include, without limitation, modulation in gene expression, modulation in transcriptional expression, or modulation in protein expression. Any method known in the art for determining gene, transcript (e.g., mRNA), and/or protein expression may be used. For example, Northern blot analysis may be used to determine anti-inflammatory mediator gene expression levels, RT-PCR may be used to determine the level of anti-inflammatory mediator transcription, and Western blot analysis may be used to determine anti-inflammatory mediator protein levels.

As used herein, the one or more genes (e.g., Fabp3, Fabp5, and/or LDR) may have modulated (e.g., increased or decreased) expression if expression in one or more cells of a subject treated with an anti-TREM2 antibody of the present disclosure is modulated as compared to the expression of the one or more genes expressed in one or more cells of a corresponding subject that is not treated with the anti-TREM2 antibody. In some embodiments, an anti-TREM2 antibody of the present disclosure may modulate gene (e.g., Fabp3, Fabp5, and/or LDR) expression in one or more cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to gene (e.g., Fabp3, Fabp5, and/or LDR) expression in one or more cells of a corresponding subject that is not treated with the anti-TREM2 antibody. In other embodiments, an anti-TREM2 antibody of the present disclosure modulates gene (e.g., Fabp3, Fabp5, and/or LDR) expression in one or more cells of a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to gene (e.g., Fabp3, Fabp5, and/or LDR) expression in one or more cells of a corresponding subject that is not treated with the anti-TREM2 antibody.

Enhancement or Normalization of the Ability of Bone Marrow-Derived Dendritic Cells to Prime or Modulate Function of Antigen-Specific T-Cells

In some embodiments, the anti-TREM2 antibodies of the present disclosure may enhance and/or normalize the ability of bone marrow-derived dendritic cells to prime or modulate function of antigen-specific T cells, including of CD8+ T cells, CD4+ T cells, and/or regulatory T cells, after binding to a TREM2 protein expressed in a cell.

In some embodiments, agonist anti-TREM2 antibodies of the present disclosure may enhance and/or normalize the ability of bone marrow-derived dendritic cells to prime or modulate function of one or more antigen-specific T cells in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the ability of bone marrow-derived dendritic cells to prime or modulate function of one or more antigen-specific T cells in a corresponding subject that is not treated with the agonist anti-TREM2 antibody. In other embodiments, the agonist anti-TREM2 antibody may enhance and/or normalize the ability of bone marrow-derived dendritic cells to prime or modulate function of antigen-specific T cells in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the ability of bone marrow-derived dendritic cells to prime or modulate function of antigen-specific T cells in a corresponding subject that is not treated with the agonist anti-TREM2 antibody.

In some embodiments, anti-TREM2 antibodies of the present disclosure may be beneficial for preventing, lowering the risk of, or treating conditions and/or diseases associated with a decreased or deregulated ability of bone marrow-derived dendritic cells to prime or modulate function of antigen-specific T cells, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibit interaction between TREM2 and one or more TREM2 ligands, and/or enhance one or more activities of one or more TREM2 ligands. Other aspects of the present disclosure relate to an agent that does not inhibit interaction between TREM2 and one or more TREM2 ligands, and/or enhance one or more activities of one or more TREM2 ligands for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

Osteoclast Production

In some embodiments, the anti-TREM2 antibodies of the present disclosure may induce osteoclast production and/or increase the rate of osteoclastogenesis after binding to a TREM2 protein expressed in a cell.

As used herein, an osteoclast is a type of bone cell that can remove bone tissue by removing its mineralized matrix and breaking up the organic bone (e.g., bone resorption). Osteoclasts can be formed by the fusion of cells of the monocyte-macrophage cell line. In some embodiments, osteoclasts may be characterized by high expression of tartrate resistant acid phosphatase (TRAP) and cathepsin K.

As used herein, the rate of osteoclastogenesis may be increased if the rate of osteoclastogenesis in a subject treated with an agonist anti-TREM2 antibody of the present disclosure is greater than the rate of osteoclastogenesis in a corresponding subject that is not treated with the agonist anti-TREM2 antibody. In some embodiments, an agonist anti-TREM2 antibody of the present disclosure may increase the rate of osteoclastogenesis in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to rate of osteoclastogenesis in a corresponding subject that is not treated with the agonist anti-TREM2 antibody. In other embodiments, an agonist anti-TREM2 antibody of the present disclosure may increase the rate of osteoclastogenesis in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to rate of osteoclastogenesis in a corresponding subject that is not treated with the agonist anti-TREM2 antibody.

As used herein, the rate of osteoclastogenesis may be decreased if the rate of osteoclastogenesis in a subject treated with an antagonist anti-TREM2 antibody of the present disclosure is smaller than the rate of osteoclastogenesis in a corresponding subject that is not treated with the antagonist anti-TREM2 antibody. In some embodiments, an antagonist anti-TREM2 antibody of the present disclosure may decrease the rate of osteoclastogenesis in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to rate of osteoclastogenesis in a corresponding subject that is not treated with the antagonist anti-TREM2 antibody. In other embodiments, an antagonist anti-TREM2 antibody of the present disclosure may decrease the rate of osteoclastogenesis in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to rate of osteoclastogenesis in a corresponding subject that is not treated with the antagonist anti-TREM2 antibody.

In some embodiments, anti-TREM2 antibodies of the present disclosure may be beneficial for preventing, lowering the risk of, or treating conditions and/or diseases associated with abnormal bone formation and maintenance including osteoporosis, which is associated with pathological decrease in bone density and osteoporotic diseases which are associated with pathological increase in bone density.

Proliferation, Survival and Functionality of TREM2-Expressing Cells

In some embodiments, the anti-TREM2 antibodies of the present disclosure may increase the proliferation, survival, and/or function of dendritic cells, macrophages, monocytes, osteoclasts, Langerhans cells of skin, Kupffer cells, and microglial cells (microglia) after binding to TREM2 protein expressed in a cell. In some embodiments, the anti-TREM2 antibodies of the present disclosure do not inhibit the growth (e.g., proliferation and/or survival) of one or more innate immune cells.

Microglial cells are a type of glial cell that are the resident macrophages of the brain and spinal cord, and thus act as the first and main form of active immune defense in the central nervous system (CNS). Microglial cells constitute 20% of the total glial cell population within the brain. Microglial cells are constantly scavenging the CNS for plaques, damaged neurons and infectious agents. The brain and spinal cord are considered “immune privileged” organs in that they are separated from the rest of the body by a series of endothelial cells known as the blood-brain barrier, which prevents most infections from reaching the vulnerable nervous tissue. In the case where infectious agents are directly introduced to the brain or cross the blood-brain barrier, microglial cells must react quickly to decrease inflammation and destroy the infectious agents before they damage the sensitive neural tissue. Due to the unavailability of antibodies from the rest of the body (few antibodies are small enough to cross the blood brain barrier), microglia must be able to recognize foreign bodies, swallow them, and act as antigen-presenting cells activating T-cells. Since this process must be done quickly to prevent potentially fatal damage, microglial cells are extremely sensitive to even small pathological changes in the CNS. They achieve this sensitivity in part by having unique potassium channels that respond to even small changes in extracellular potassium.

As used herein, macrophages of the present disclosure include, without limitation, M1 macrophages, activated M1 macrophages, and M2 macrophages. As used herein, microglial cells of the present disclosure include, without limitation, M1 microglial cells, activated M1 microglial cells, and M2 microglial cells. In some embodiments, anti-TREM2 antibodies of the present disclosure may be beneficial for, lowering the risk of, or treating conditions and/or diseases associated with decreased proliferation or survival, of immune cells, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibit interaction between TREM2 and one or more TREM2 ligands, and/or enhance one or more activities of one or more TREM2 ligands. Other aspects of the present disclosure relate to an agent that does not inhibit interaction between TREM2 and one or more TREM2 ligands, and/or enhance one or more activities of one or more TREM2 ligands for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

In some embodiments, anti-TREM2 antibodies of the present disclosure may increase the expression of CD83 and/or CD86 on dendritic cells, monocytes, and/or macrophages.

As used herein, the rate of proliferation, survival, and/or function of macrophages, dendritic cells, monocytes, and/or microglia may include increased expression if the rate of proliferation, survival, and/or function of dendritic cells, macrophages, monocytes, osteoclasts, Langerhans cells of skin, Kupffer cells, and/or microglia in a subject treated with an anti-TREM2 antibody of the present disclosure is greater than the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody. In some embodiments, an anti-TREM2 antibody of the present disclosure may increase the rate of proliferation, survival, and/or function of dendritic cells, macrophages, monocytes, osteoclasts, Langerhans cells of skin, Kupffer cells, and/or microglia in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody. In other embodiments, an anti-TREM2 antibody of the present disclosure may increase the rate of proliferation, survival, and/or function of dendritic cells, macrophages, monocytes, osteoclasts, Langerhans cells of skin, Kupffer cells, and/or microglia in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody.

In some embodiments, anti-TREM2 antibodies of the present disclosure may be beneficial for preventing, lowering the risk of, or treating conditions and/or diseases associated with a reduction in function of dendritic cells, macrophages, monocytes, osteoclasts, Langerhans cells of skin, Kupffer cells, and/or microglia including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibit interaction between TREM2 and one or more TREM2 ligands, and/or enhance one or more activities of at least one TREM2 ligand. Other aspects of the present disclosure relate to an agent that does not inhibit interaction between TREM2 and one or more TREM2 ligands, and/or enhance one or more activities of at least one TREM2 ligand for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

Clearance and Phagocytosis

In some embodiments, the anti-TREM2 antibodies of the present disclosure may induce clearance and/or phagocytosis after binding to a TREM2 protein expressed in a cell of one or more of apoptotic neurons, nerve tissue debris of the nervous system, non-nerve tissue debris of the nervous system, bacteria, other foreign bodies, disease-causing proteins, disease-causing peptides, disease-causing nucleic acid, or tumor cells. In certain embodiments, disease-causing proteins include, without limitation, amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products including DiPeptide Repeats, (DPRs peptides) composed of glycine-alanine (GA), glycine-proline (GP), glycine-arginine (GR), proline-alanine (PA), or proline-arginine (PR). In certain embodiments, disease-causing nucleic acids include, without limitation, antisense GGCCCC (G2C4) repeat-expansion RNA.

In some embodiments, the anti-TREM2 antibodies of the present disclosure may induce of one or more types of clearance, including without limitation, apoptotic neuron clearance, nerve tissue debris clearance, non-nerve tissue debris clearance, bacteria or other foreign body clearance, disease-causing protein clearance, disease-causing peptide clearance, disease-causing nucleic acid clearance, and tumor cell clearance.

In some embodiments, the anti-TREM2 antibodies of the present disclosure may induce phagocytosis of one or more of apoptotic neurons, nerve tissue debris, non-nerve tissue debris, bacteria, other foreign bodies, disease-causing proteins, disease-causing peptides, disease-causing nucleic acid, and/or tumor cells.

In some embodiments, the anti-TREM2 antibodies of the present disclosure may increase phagocytosis by macrophages, dendritic cells, monocytes, and/or microglia under conditions of reduced levels of macrophage colony-stimulating factor (MCSF). Alternatively, in some embodiments, the anti-TREM2 antibodies of the present disclosure may increase phagocytosis by macrophages, dendritic cells, monocytes, and/or microglia in the presence of normal levels of macrophage colony-stimulating factor (MCSF)

In some embodiments, anti-TREM2 antibodies of the present disclosure may be beneficial for preventing, lowering the risk of, or treating conditions and/or diseases associated with clearance and/or phagocytosis of apoptotic neurons, nerve tissue debris of the nervous system, non-nerve tissue debris of the nervous system, bacteria, other foreign bodies, disease-causing proteins, disease-causing peptides, disease-causing nucleic acid, or tumor cells., including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibit interaction between TREM2 and one or more TREM2 ligand, and/or enhance one or more activities of at least one TREM2 ligand. Other aspects of the present disclosure relate to an agent does not inhibit interaction between TREM2 and i for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

TREM2-Dependent Gene Expression

In some embodiments, agonist anti-TREM2 antibodies of the present disclosure may increase the activity and/or expression of TREM2-dependent genes, such as one or more transcription factors of the nuclear factor of activated T-cells (NFAT) family of transcription factors. Alternatively, antagonistic anti-TREM2 antibodies of the present disclosure may inhibit the activity and/or expression of TREM2-dependent genes, such as one or more transcription factors of the NFAT family of transcription factors.

In some embodiments, anti-TREM2 antibodies of the present disclosure may be beneficial for preventing, lowering the risk of, or treating conditions and/or diseases associated with decreased levels of TREM2-dependent genes, including dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, comprising administering to an individual in need thereof a therapeutically effective amount of an agent that does not inhibit interaction between TREM2 and one or more TREM2 ligands, and/or enhance one or more activities of at least one TREM2 ligand. Other aspects of the present disclosure relate to an agent that does not inhibit interaction between TREM2 and one or more CD33 ligands for use in preventing, reducing risk, or treating a disease, disorder, or injury selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza.

Antibody Preparation

Anti-TREM2 antibodies of the present disclosure can encompass polyclonal antibodies, monoclonal antibodies, humanized and chimeric antibodies, human antibodies, antibody fragments (e.g., Fab, Fab′-SH, Fv, scFv, and F(ab′)₂), bispecific and polyspecific antibodies, multivalent antibodies, library derived antibodies, antibodies having modified effector functions, fusion proteins containing an antibody portion, and any other modified configuration of the immunoglobulin molecule that includes an antigen recognition site, such as an epitope having amino acid residues of a TREM2 protein of the present disclosure, including glycosylation variants of antibodies, amino acid sequence variants of antibodies, and covalently modified antibodies. The anti-TREM2 antibodies may be human, murine, rat, or of any other origin (including chimeric or humanized antibodies).

(1) Polyclonal Antibodies

Polyclonal antibodies, such as anti-TREM2 polyclonal antibodies, are generally raised in animals by multiple subcutaneous (sc) or intraperitoneal (ip) injections of the relevant antigen and an adjuvant. It may be useful to conjugate the relevant antigen (e.g., a purified or recombinant TREM2 protein of the present disclosure) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R′ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 μg (for rabbits) or 5 μg (for mice) of the protein or conjugate with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant-cell culture as protein fusions. Also, aggregating agents such as alum are suitable to enhance the immune response.

(2) Monoclonal Antibodies

Monoclonal antibodies, such as anti-TREM2 monoclonal antibodies, are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the anti-TREM2 monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization (e.g., a purified or recombinant TREM2 protein of the present disclosure). Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The immunizing agent will typically include the antigenic protein (e.g., a purified or recombinant TREM2 protein of the present disclosure) or a fusion variant thereof. Generally peripheral blood lymphocytes (“PBLs”) are used if cells of human origin are desired, while spleen or lymph node cells are used if non-human mammalian sources are desired. The lymphoctyes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine or human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient-cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors (available from the Salk Institute Cell Distribution Center, San Diego, Calif. USA), as well as SP-2 cells and derivatives thereof (e.g., X63-Ag8-653) (available from the American Type Culture Collection, Manassas, Va. USA). Human myeloma and mouse-human heteromyeloma cell lines have also been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen (e.g., a TREM2 protein of the present disclosure). Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen (e.g., a TREM2 protein of the present disclosure). Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Anti-TREM2 monoclonal antibodies may also be made by recombinant DNA methods, such as those disclosed in U.S. Pat. No. 4,816,567, and as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host-cells such as E. coli cells, simian COS cells, Chinese hamster ovary (CHO) cells, or myeloma cells that do not otherwise produce immunoglobulin protein, in order to synthesize monoclonal antibodies in such recombinant host-cells. Review articles on recombinant expression in bacteria of DNA encoding the antibody include Skerra et al., Curr. Opin. Immunol., 5:256-262 (1993) and Plückthun, Immunol. Rev. 130:151-188 (1992).

In certain embodiments, anti-TREM2 antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies of desired specificity (e.g., those that bind a TREM2 protein of the present disclosure).

The DNA encoding antibodies or fragments thereof may also be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein (e.g., anti-TREM2 antibodies of the present disclosure or fragments thereof) may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid anti-TREM2 antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

(3) Humanized Antibodies

Anti-TREM2 antibodies of the present disclosure or antibody fragments thereof may further include humanized or human antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally will also comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Methods for humanizing non-human anti-TREM2 antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. hi practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Furthermore, it is important that antibodies be humanized with retention of high affinity for the antigen and other favorable biological properties. To achieve this goal, according to a preferred method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., TREM2 proteins of the present disclosure), is achieved. In general, the CDR residues are directly and most substantially involved in influencing antigen binding.

Various forms of the humanized anti-TREM2 antibody are contemplated. For example, the humanized anti-TREM2 antibody may be an antibody fragment, such as an Fab, which is optionally conjugated with one or more TREM2 ligand, such as HSP60. Alternatively, the humanized anti-TREM2 antibody may be an intact antibody, such as an intact IgG1 antibody.

(4) Human Antibodies

Alternatively, human anti-TREM2 antibodies can be generated. For example, it is now possible to produce transgenic animals (e.g., mice) that are capable, upon immunization, of producing a full repertoire of human antibodies in the absence of endogenous immunoglobulin production. The homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Nat'l Acad. Sci. USA, 90:2551 (1993); Jakobovits et al., Nature, 362:255-258 (1993); Bruggermann et al., Year in Immunol., 7:33 (1993); U.S. Pat. No. 5,591,669 and WO 97/17852.

Alternatively, phage display technology can be used to produce human anti-TREM2 antibodies and antibody fragments in vitro, from immunoglobulin variable (V) domain gene repertoires from unimmunized donors. McCafferty et al., Nature 348:552-553 (1990); Hoogenboom and Winter, J. Mol. Biol. 227: 381 (1991). According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Curr. Opin Struct. Biol. 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See also U.S. Pat. Nos. 5,565,332 and 5,573,905. Additionally, yeast display technology can be used to produce human anti-TREM2 antibodies and antibody fragments in vitro (e.g., WO 2009/036379; WO 2010/105256; WO 2012/009568; US 2009/0181855; US 2010/0056386; and Feldhaus and Siegel (2004) J. Immunological Methods 290:69-80). In other embodiments, ribosome display technology can be used to produce human anti-TREM2 antibodies and antibody fragments in vitro (e.g., Roberts and Szostak (1997) Proc Natl Acad Sci 94:12297-12302; Schaffitzel et al. (1999) J. Immunolical Methods 231:119-135; Lipovsek and Plückthun (2004) J. Immunological Methods 290:51-67).

The techniques of Cole et al., and Boerner et al., are also available for the preparation of human anti-TREM2 monoclonal antibodies (Cole et al., Monoclonal Antibodies and Cancer Therapy, Alan R. Liss, p. 77 (1985) and Boerner et al., J. Immunol. 147(1): 86-95 (1991). Similarly, human anti-TREM2 antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806, 5,569,825, 5,625,126, 5,633,425, 5,661,016 and in the following scientific publications: Marks et al., Bio/Technology 10: 779-783 (1992); Lonberg et al., Nature 368: 856-859 (1994); Morrison, Nature 368: 812-13 (1994), Fishwild et al., Nature Biotechnology 14: 845-51 (1996), Neuberger, Nature Biotechnology 14: 826 (1996) and Lonberg and Huszar, Intern. Rev. Immunol. 13: 65-93 (1995).

Finally, human anti-TREM2 antibodies may also be generated in vitro by activated B-cells (see U.S. Pat. Nos. 5,567,610 and 5,229,275).

(5) Antibody Fragments

In certain embodiments there are advantages to using anti-TREM2 antibody fragments, rather than whole anti-TREM2 antibodies. In some embodiments, smaller fragment sizes allow for rapid clearance and better brain penetration.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host-cells, for example, using nucleic acids encoding anti-TREM2 antibodies of the present disclosure. Fab, Fv and scFv antibody fragments can all be expressed in and secreted from E. coli, thus allowing the straightforward production of large amounts of these fragments. Anti-TREM2 antibody fragments can also be isolated from the antibody phage libraries as discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host-cell culture. Production of Fab and F(ab′)₂ antibody fragments with increased in vivo half-lives are described in U.S. Pat. No. 5,869,046. In other embodiments, the antibody of choice is a single chain Fv fragment (scFv). See WO 93/16185; U.S. Pat. Nos. 5,571,894 and 5,587,458. The anti-TREM2 antibody fragment may also be a “linear antibody,” e.g., as described in U.S. Pat. No. 5,641,870. Such linear antibody fragments may be monospecific or bispecific.

(6) Bispecific and Polyspecific Antibodies

Bispecific antibodies (BsAbs) are antibodies that have binding specificities for at least two different epitopes, including those on the same or another protein (e.g., one or more TREM2 proteins of the present disclosure). Alternatively, one part of a BsAb can be armed to bind to the target TREM2 antigen, and another can be combined with an arm that binds to a second protein. Such antibodies can be derived from full-length antibodies or antibody fragments (e.g., F(ab′)₂ bispecific antibodies).

Methods for making bispecific antibodies are known in the art. Traditional production of full-length bispecific antibodies is based on the coexpression of two immunoglobulin heavy-chain/light chain pairs, where the two chains have different specificities. Millstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only half of the bispecific molecules provides for an easy way of separation. This approach is disclosed in WO 94/04690. For further details of generating bispecific antibodies, see, for example, Suresh et al., Methods in Enzymology 121: 210 (1986); and Garber, Nature Reviews Drug Discovery 13, 799-801 (2014).

According to another approach described in WO 96/27011 or U.S. Pat. No. 5,731,168, the interface between a pair of antibody molecules can be engineered to maximize the percentage of heterodimers which are recovered from recombinant-cell culture. The preferred interface comprises at least a part of the C_{H}3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T-cells, as well as trigger the lytic activity of human cytotoxic lymphocytes against human breast tumor targets.

Various techniques for making and isolating bivalent antibody fragments directly from recombinant-cell culture have also been described. For example, bivalent heterodimers have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Nat'l Acad. Sci. USA, 90: 6444-6448 (1993) has provided an alternative mechanism for making bispecific/bivalent antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific/bivalent antibody fragments by the use of single-chain Fv (sFv) dimers has also been reported. See Gruber et al., J. Immunol., 152:5368 (1994).

Another method to generate bispecific antibodies is designated controlled Fab-arm exchange (cFAE), which is an easy-to-use method to generate bispecific IgG1 (bslgGl). The protocol involves the following: (i) separate expression of two parental IgGls containing single matching point mutations in the CH3 domain; (ii) mixing of parental IgGls under permissive redox conditions in vitro to enable recombination of half-molecules; (iii) removal of the reductant to allow reoxidation of interchain disulfide bonds; and (iv) analysis of exchange efficiency and final product using chromatography-based or mass spectrometry (MS)-based methods. The protocol generates bsAbs with regular IgG architecture, characteristics and quality attributes both at bench scale (micrograms to milligrams) and at a mini-bioreactor scale (milligrams to grams) that is designed to model large-scale manufacturing (kilograms). Starting from good-quality purified proteins, exchange efficiencies of >95% can be obtained within 2-3 days (including quality control). See Labrijn et al., Natur Protocols 9, 2450-2463 (2014); and Garber, Nature Reviews Drug Discovery 13, 799-801 (2014).

Antibodies with more than two valencies are also contemplated. For example, trispecific antibodies can be prepared. Tutt et al., J. Immunol. 147:60 (1991).

Exemplary bispecific antibodies may bind to two different epitopes on a given molecule (e.g., a TREM2 protein of the present disclosure). In some embodiments a bispecific antibody binds to a first antigen, such as a TREM2 or DAP12 protein of the present disclosure, and a second antigen facilitating transport across the blood-brain barrier. Numerous antigens are known in the art that facilitate transport across the blood-brain barrier (see, e.g., Gabathuler R., Approaches to transport therapeutic drugs across the blood-brain barrier to treat brain diseases, Neurobiol. Dis. 37 (2010) 48-57). Such second antigens include, without limitation, transferrin receptor (TR), insulin receptor (HIR), Insulin-like growth factor receptor (IGFR), low-density lipoprotein receptor related proteins 1 and 2 (LPR-1 and 2), diphtheria toxin receptor, including CRM197 (a non-toxic mutant of diphtheria toxin), llama single domain antibodies such as TMEM 30(A) (Flippase), protein transduction domains such as TAT, Syn-B, or penetratin, poly-arginine or generally positively charged peptides, Angiopep peptides such as ANG1005 (see, e.g., Gabathuler, 2010), and other cell surface proteins that are enriched on blood-brain barrier endothelial cells (see, e.g., Daneman et al., PLoS One. 2010 Oct. 29; 5(10):e13741). In some embodiments, second antigens for an anti-TREM2 antibody may include, without limitation, a DAP12 antigen of the present disclosure. In other embodiments, second antigens for an anti-DAP12 antibody may include, without limitation, a TREM2 antigen of the present disclosure. In other embodiments, bispecific antibodies that bind to both TREM2 and DAP12 may facilitate and enhance one or more TREM2 activities. In other embodiments, second antigens for an anti-TREM2 antibody may include, without limitation, A beta peptide, antigen or an alpha synuclein protein antigen or, Tau protein antigen or, TDP-43 protein antigen or, prion protein antigen or, huntingtin protein antigen, or RAN, translation Products antigen, including the DiPeptide Repeats, (DPRs peptides) composed of glycine-alanine (GA), glycine-proline (GP), glycine-arginine (GR), proline-alanine (PA), or proline-arginine (PR).

(7) Multivalent Antibodies

A multivalent antibody may be internalized (and/or catabolized) faster than a bivalent antibody by a cell expressing an antigen to which the antibodies bind. The anti-TREM2 antibodies of the present disclosure or antibody fragments thereof can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises an Fc region or a hinge region. In this scenario, the antibody will comprise an Fc region and three or more antigen binding sites amino-terminal to the Fc region. The preferred multivalent antibody herein contains three to about eight, but preferably four, antigen binding sites. The multivalent antibody contains at least one polypeptide chain (and preferably two polypeptide chains), wherein the polypeptide chain or chains comprise two or more variable domains. For instance, the polypeptide chain or chains may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}—C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain. The Multivalent antibodies may recognize the TREM2 antigen as well as without limitation additional antigens A beta peptide, antigen or an alpha synuclein protein antigen or, Tau protein antigen or, TDP-43 protein antigen or, prion protein antigen or, huntingtin protein antigen, or RAN, translation Products antigen, including the DiPeptide Repeats, (DPRs peptides) composed of glycine-alanine (GA), glycine-proline (GP), glycine-arginine (GR), proline-alanine (PA), or proline-arginine (PR), Insulin receptor, insulin like growth factor receptor. Transferrin receptor or any other antigen that facilitate antibody transfer across the blood brain barrier.

(8) Effector Function Engineering

It may also be desirable to modify an anti-TREM2 antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH 2 domain of IgG) of the antibody. In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000). In other embodiments, it may also be desirable to modify an anti-TREM2 antibody of the present disclosure to modify effector function to increase finding selectivity toward the ITIM-containing FcgRIIb (CD32b) to increase clustering of TREM2 antibodies on adjacent cells without activating humoral responses including Antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

(9) Other Amino Acid Sequence Modifications

Amino acid sequence modifications of anti-TREM2 antibodies of the present disclosure, or antibody fragments thereof, are also contemplated. For example, it may be desirable to improve the binding affinity and/or other biological properties of the antibodies or antibody fragments. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics (i.e., the ability to bind or physically interact with a TREM2 protein of the present disclosure). The amino acid changes also may alter post-translational processes of the antibody, such as changing the number or position of glycosylation sites.

A useful method for identification of certain residues or regions of the anti-TREM2 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the target antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity.

Amino acid sequence insertions include amino-(“N”) and/or carboxy-(“C”) terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table C below under the heading of “preferred substitutions”. If such substitutions result in a change in biological activity, then more substantial changes, denominated “exemplary substitutions” in Table C, or as further described below in reference to amino acid classes, may be introduced and the products screened.

TABLE C
Amino Acid Substitutions

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | Ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions entail exchanging a member of one of these classes for another class.

Any cysteine residue not involved in maintaining the proper conformation of the antibody also may be substituted, generally with serine, to improve the oxidative stability of the molecule and prevent aberrant crosslinking Conversely, cysteine bond(s) may be added to the antibody to improve its stability (particularly where the antibody is an antibody fragment, such as an Fv fragment).

A particularly preferred type of substitutional variant involves substituting one or more hypervariable region residues of a parent antibody (e.g. a humanized or human anti-TREM2 antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen (e.g., a TREM2 protein of the present disclosure). Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-IgE antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibodies (e.g., anti-TREM2 antibodies of the present disclosure) or antibody fragments.

(10) Other Antibody Modifications

Anti-TREM2 antibodies of the present disclosure, or antibody fragments thereof, can be further modified to contain additional non-proteinaceous moieties that are known in the art and readily available, or to contain different types of drug conjugates that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

Drug conjugation involves coupling of a biological active cytotoxic (anticancer) payload or drug to an antibody that specifically targets a certain tumor marker (e.g. a protein that, ideally, is only to be found in or on rumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue, OncLive Jul. 5, 2012; ADC Review on antibody-drug conjugates; and Ducry et al., (2010). Bioconjugate Chemistry 21 (1): 5-13).

Binding Assays and Other Assays

Anti-TREM2 antibodies of the present disclosure may be tested for antigen binding activity, e.g., by known methods such as ELISA, Western blot, etc.

In some embodiments, competition assays may be used to identify an antibody that competes with any of the antibodies listed in Tables 2A, 2B, 3A, 3B, 4A, 4B, 7A, and 7B, selected from 1A7, 3A2, 3B10, 6G12, 6H6, 7A9, 7B3, 8A1, 8E10, 8F11, 8F8, 9F5, 9G1, 9G3, 10A9, 10C1, 11A8, 12E2, 12F9, 12G6, 2C7, 2F5, 3C1, 4D7, 4D11, 6C11, 6G12, 7A3, 7C5, 7E9, 7F6, 7G1, 7H1, 8C3, 8F10, 12A1, 1E9, 2C5, 3C5, 4C12, 4F2, 5A2, 6B3, 7D1, 7D9, 11D8, 8A12, 10E7, 10B11, 10D2, 7D5, 2A7, 3G12, 6H9, 8G9, 9B4, 10A1, 11A8, 12F3, 2F8, 10E3, 1H7, 2F6, 2H8, 3A7, 7E5, 7F8, 11H5, 7C5, 4F11, 12D9, 1B4v1, 1B4v2, 6H2, 7B11v1, 7B11v2, 18D8, 18E4v1, 18E4v2, 29F6v1, 29F6v2, 40D5v1, 40D5v2, 43B9, 44A8v1, 44A8v2, 44B4v1, and 44B4v2 and humanized variants thereof, and/or humanized antibody M7E57291 for binding to TREM2. In certain embodiments, such a competing antibody binds to the same epitope (e.g., a linear or a conformational epitope) that is bound by any of the antibodies listed in Table 1, selected from 4D11, 7C5, 6G12, 8F11, 8E10, 7E5, 7F8, 8F8, 1H7. 2H8. 3A2. 3A7. 3B10, 4F11, 6H6, 7A9, 7B3, 8A1, 9F5, 9G1, 9G3, 10A9, 11A8, 12D9, 12F9, 1B4v1, 1B4v2, 6H2, 7B11v1, 7B11v2, 18D8, 18E4v1, 18E4v2, and their humanized derivatives, and/or human and/or humanized M7E57291. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized TREM2 or cells expressing TREM2 on cell surface are incubated in a solution comprising a first labeled antibody that binds to TREM2 (e.g., human or non-human primate) and a second unlabeled antibody that is being tested for its ability to compete with the first antibody for binding to TREM2. The second antibody may be present in a hybridoma supernatant. As a control, immobilized TREM2 or cells expressing TREM2 is incubated in a solution comprising the first labeled antibody but not the second unlabeled antibody. After incubation under conditions permissive for binding of the first antibody to TREM2, excess unbound antibody is removed, and the amount of label associated with immobilized TREM2 or cells expressing TREM2 is measured. If the amount of label associated with immobilized TREM2 or cells expressing TREM2 is substantially reduced in the test sample relative to the control sample, then that indicates that the second antibody is competing with the first antibody for binding to TREM2. See Harlow and Lane (1988) Antibodies: A Laboratory Manual ch.14 (Cold Spring Harbor Laboratory, Cold Spring Harbor, N.Y.).

### Nucleic Acids, Vectors, and Host Cells

Anti-TREM2 antibodies of the present disclosure may be produced using recombinant methods and compositions, e.g., as described in U.S. Pat. No. 4,816,567. In some embodiments, isolated nucleic acids having a nucleotide sequence encoding any of the anti-TREM2 antibodies of the present disclosure are provided. Such nucleic acids may encode an amino acid sequence containing the VL and/or an amino acid sequence containing the VH of the anti-TREM2 antibody (e.g., the light and/or heavy chains of the antibody). In some embodiments, one or more vectors (e.g., expression vectors) containing such nucleic acids are provided. In some embodiments, a host cell containing such nucleic acid is also provided. In some embodiments, the host cell contains (e.g., has been transduced with): (1) a vector containing a nucleic acid that encodes an amino acid sequence containing the VL of the antibody and an amino acid sequence containing the VH of the antibody, or (2) a first vector containing a nucleic acid that encodes an amino acid sequence containing the VL of the antibody and a second vector containing a nucleic acid that encodes an amino acid sequence containing the VH of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-TREM2 antibody of the present disclosure are provided. In some embodiments, the method includes culturing a host cell of the present disclosure containing a nucleic acid encoding the anti-TREM2 antibody, under conditions suitable for expression of the antibody. In some embodiments, the antibody is subsequently recovered from the host cell (or host cell culture medium).

For recombinant production of an anti-TREM2 antibody of the present disclosure, a nucleic acid encoding the anti-TREM2 antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the anti-TREM2 antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColEl, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-TREM2 antibody of the present disclosure.

Suitable host cells for cloning or expression of antibody-encoding vectors include prokaryotic or eukaryotic cells. For example, anti-TREM2 antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross, Nat. Biotech. 22:1409-1414 (2004); and Li et al., Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants.).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Pharmaceutical Compositions

Anti-TREM2 antibodies of the present disclosure can be incorporated into a variety of formulations for therapeutic administration by combining the antibodies with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

A pharmaceutical composition of the present disclosure can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include, without limitation, sulfate, gluconate, citrate and phosphate. The polypeptides of a composition can also be complexed with molecules that enhance their in vivo attributes. Such molecules include, without limitation, carbohydrates, polyamines, amino acids, other peptides, ions (e.g., sodium, potassium, calcium, magnesium, manganese), and lipids.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the antibody, such as an anti-TREM2 antibody of the present disclosure, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

Pharmaceutical Dosages

Pharmaceutical compositions of the present disclosure containing an anti-TREM2 antibody of the present disclosure may be administered to an individual in need of treatment with the anti-TREM2 antibody, preferably a human, in accord with known methods, such as intravenous administration as a bolus or by continuous infusion over a period of time, by intramuscular, intraperitoneal, intracerobrospinal, intracranial, intraspinal, subcutaneous, intra-articular, intrasynovial, intrathecal, oral, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the anti-TREM2 antibodies of the present disclosure, normal dosage amounts may vary from about 10 ng/kg up to about 100 mg/kg of an individual's body weight or more per day, preferably about 1 mg/kg/day to 10 mg/kg/day, depending upon the route of administration. For repeated administrations over several days or longer, depending on the severity of the disease, disorder, or condition to be treated, the treatment is sustained until a desired suppression of symptoms is achieved.

An exemplary dosing regimen may include administering an initial dose of an anti-TREM2 antibody, of about 2 mg/kg, followed by a weekly maintenance dose of about 1 mg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, and about 2/mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the anti-TREM2 antibody administered, can vary over time independently of the dose used.

Dosages for a particular anti-TREM2 antibody may be determined empirically in individuals who have been given one or more administrations of the anti-TREM2 antibody. Individuals are given incremental doses of an anti-TREM2 antibody. To assess efficacy of an anti-TREM2 antibody, a clinical symptom of ay of the diseases, disorders, or conditions of the present disclosure (e.g., dementia, frontotemporal dementia, Alzheimer's disease, Nasu-Hakola disease, and multiple sclerosis) can be monitored.

Administration of an anti-TREM2 antibody of the present disclosure can be continuous or intermittent, depending, for example, on the recipient's physiological condition, whether the purpose of the administration is therapeutic or prophylactic, and other factors known to skilled practitioners. The administration of an anti-TREM2 antibody may be essentially continuous over a preselected period of time or may be in a series of spaced doses.

Guidance regarding particular dosages and methods of delivery is provided in the literature; see, for example, U.S. Pat. No. 4,657,760; 5,206,344; or 5,225,212. It is within the scope of the present disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Therapeutic Uses

Further aspects of the present disclosure provide methods for modulating (e.g., activating or inhibiting) TREM2, modulating (e.g., activating or inhibiting) DAP12, modulating (e.g., activating or inhibiting) PI3K, modulating (e.g., increasing or reducing) expression of one or more pro- and anti-inflammatory mediators (e.g., IFN-α4, IFN-b, IL-1(3, TNF-α, IL-10, IL-6, IL-8, IL-23, TGF-beta members of the chemokine protein families, IL-20 family members, IL-33, LIF, IFN-gamma, OSM, CNTF, TGF-beta, GM-CSF, IL-11, IL-12, IL-17, IL-18, IL-23, CCL4, MCP-1, VEGF, CXCL10 and CRP) or, modulating (e.g., increasing or reducing) survival of one or more TREM2 expressing cells or, modulating (e.g., increasing or reducing) functionality of one or more TREM2 expressing cells, or, modulating (e.g., increasing or reducing) proliferation of one or more TREM2 expressing cells or, or, modulating (e.g., increasing or reducing) migration of one or more TREM2 expressing cells, or, modulating (e.g., increasing or reducing) interaction with other cells of one or more TREM2 expressing cells in an individual in need thereof, by administering to the individual a therapeutically effective amount of an anti-TREM2 antibody of the present disclosure to modulate (e.g., induce or inhibit) one or more TREM2 activities in the individual.

As disclosed herein, anti-TREM2 antibodies of the present disclosure may be used for preventing, reducing risk, or treating dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and/or Haemophilus influenza. In some embodiments, the anti-TREM2 antibodies are agonist antibodies.

In some embodiments, the present disclosure provides methods of preventing, reducing risk, or treating an individual having dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, tauopathy disease, Nasu-Hakola disease, stroke, acute trauma, chronic trauma, cognitive deficit, memory loss, lupus, acute and chronic colitis, rheumatoid arthritis, wound healing, Crohn's disease, inflammatory bowel disease, ulcerative colitis, obesity, malaria, essential tremor, central nervous system lupus, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, sarcoidosis, diseases of aging, seizures, spinal cord injury, traumatic brain injury, age related macular degeneration, glaucoma, retinitis pigmentosa, retinal degeneration, respiratory tract infection, sepsis, eye infection, systemic infection, lupus, arthritis, multiple sclerosis, low bone density, osteoporosis, osteogenesis, osteopetrotic disease, Paget's disease of bone, cancer, bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, acute lymphoblastic leukemia (ALL), acute myeloid leukemia (AML), chronic lymphocytic leukemia (CLL), chronic myeloid leukemia (CML), multiple myeloma, polycythemia vera, essential thrombocytosis, primary or idiopathic myelofibrosis, primary or idiopathic myelosclerosis, myeloid-derived tumors, tumors that express TREM2, thyroid cancer, infections, CNS herpes, parasitic infections, Trypanosome infection, Cruzi infection, Pseudomonas aeruginosa infection, Leishmania donovani infection, group B Streptococcus infection, Campylobacter jejuni infection, Neisseria meningiditis infection, type I HIV, and Haemophilus influenza, by administering to the individual a therapeutically effective amount of an anti-TREM2 antibody of the present disclosure. In some embodiments, the anti-TREM2 antibody is an agonist antibody. In some embodiments, the anti-TREM2 antibody is an inert antibody. In some embodiments, the anti-TREM2 antibody is an antagonist antibody. In some embodiments, the method further includes administering to the individual at least one antibody that specifically binds to an inhibitory checkpoint molecule, and/or another standard or investigational anti-cancer therapy. In some embodiments, the antibody that specifically binds to an inhibitory checkpoint molecule is administered in combination with the isolated antibody. In some embodiments, the at least one antibody that specifically binds to an inhibitory checkpoint molecule is selected from an anti-PD-L1 antibody, an anti-CTLA-4 antibody, an anti-PD-L2 antibody, an anti-PD-1 antibody, an anti-B7-H3 antibody, an anti-B7-H4 antibody, and anti-HVEM antibody, an anti-B- and T-lymphocyte attenuator (BTLA) antibody, an anti-Killer inhibitory receptor (KIR) antibody, an anti-GALS antibody, an anti-TIM3 antibody, an anti-AZAR antibody, an anti-LAG-3 antibody, an anti-phosphatidylserine antibody, an anti-CD27 antibody, and any combination thereof. In some embodiments, the standard or investigational anti-cancer therapy is one or more therapies selected from radiotherapy, cytotoxic chemotherapy, targeted therapy, hormonal therapy, imatinib (Gleevec®), trastuzumab (Herceptin®), bevacizumab (Avastin®), Ofatumumab (Arzerra®), Rituximab (Rituxan®, MabThera®, Zytux®), cryotherapy, ablation, radiofrequency ablation, adoptive cell transfer (ACT), chimeric antigen receptor T cell transfer (CAR-T), vaccine therapy, and cytokine therapy. In some embodiments, the method further includes administering to the individual at least one antibody that specifically binds to an inhibitory cytokine. In some embodiments, the at least one antibody that specifically binds to an inhibitory cytokine is administered in combination with the isolated antibody. In some embodiments, the at least one antibody that specifically binds to an inhibitory cytokine is selected from an anti-CCL2 antibody, an anti-CSF-1 antibody, an anti-IL-2 antibody, and any combination thereof. In some embodiments, the method further includes administering to the individual at least one agonistic antibody that specifically binds to a stimulatory checkpoint protein. In some embodiments, the at least one agonistic antibody that specifically binds to a stimulatory checkpoint protein is administered in combination with the isolated antibody. In some embodiments, the at least one agonistic antibody that specifically binds to a stimulatory checkpoint protein is selected from an agonist anti-CD40 antibody, an agonist anti-0X40 antibody, an agonist anti-ICOS antibody, an agonist anti-CD28 antibody, an agonist anti-CD137/4-1BB antibody, an agonist anti-CD27 antibody, an agonist anti-glucocorticoid-induced TNFR-related protein GITR antibody, and any combination thereof. In some embodiments, the method further includes administering to the individual at least one stimulatory cytokine. In some embodiments, the at least one stimulatory cytokine is administered in combination with the isolated antibody. In some embodiments, the at least one stimulatory cytokine is selected from TNF-α, IL-10, IL-6, IL-8, CRP, TGF-beta members of the chemokine protein families, IL20 family member, IL-33, LIF, OSM, CNTF, TGF-beta, IL-11, IL-12, IL-17, IL-8, IL-23, IFN-α, IFN-β, IL-2, IL-18, GM-CSF, G-CSF, and any combination thereof.

In some embodiments, the present disclosure provides methods of preventing, reducing risk, or treating an individual having Alzheimer's disease by administering to the individual a therapeutically effective amount of an anti-TREM2 antibody of the present disclosure. In some embodiments, the anti-TREM2 antibody is an agonist antibody. In some embodiments, the anti-TREM2 antibody increases expression of one or more inflammatory mediators, such as IL-1β, TNF-α, YM-1, CD86, CCL2, CCL3, CCL5, CCR2, CXCL10, Gata3, Rorc, and any combination thereof. In some embodiments, the anti-TREM2 antibody decreases expression of one or more inflammatory mediators, such as FLT1, OPN, CSF-1, CD11c, AXL, and any combination thereof. In some embodiments, the anti-TREM2 antibody decreases levels of Abeta peptide in the individual (e.g., in the brain of the individual). In some embodiments, the anti-TREM2 antibody increases the number of CD11b⁺ microglial cells in the brain of the individual. In some embodiments, the anti-TREM2 antibody increases memory in the individual. In some embodiments, the anti-TREM2 antibody reduces cognitive deficit in the individual. In some embodiments, the anti-TREM2 antibody increases motor coordination in the individual.

In some embodiments, the present disclosure provides methods of increasing memory, reducing cognitive deficit, or both in an individual in need thereof, by administering to the individual a therapeutically effective amount of an anti-TREM2 antibody of the present disclosure. In some embodiments, the anti-TREM2 antibody is an agonist antibody.

In some embodiments, the present disclosure provides methods of increasing motor coordination in an individual in need thereof, by administering to the individual a therapeutically effective amount of an anti-TREM2 antibody of the present disclosure. In some embodiments, the anti-TREM2 antibody is an agonist antibody.

In some embodiments, the present disclosure provides methods of reducing Abeta peptide levels in an individual in need thereof, by administering to the individual a therapeutically effective amount of an anti-TREM2 antibody of the present disclosure. In some embodiments, the anti-TREM2 antibody is an agonist antibody.

In some embodiments, the present disclosure provides methods of increasing the number of CD11b⁺ microglial cells in an individual in need thereof, by administering to the individual a therapeutically effective amount of an anti-TREM2 antibody of the present disclosure. In some embodiments, the anti-TREM2 antibody is an agonist antibody.

In some embodiments, the present disclosure provides methods of increasing levels of one or more of FLT1, OPNCSF1, CD11c, and AXL in an individual in need thereof, by administering to the individual a therapeutically effective amount of an anti-TREM2 antibody of the present disclosure. In some embodiments, the anti-TREM2 antibody is an agonist antibody.

In some embodiments, an anti-TREM2 antibody of the present disclosure may increases expression of one or more inflammatory mediators, such as IL-1β, TNF-α, YM-1, CD86, CCL2, CCL3, CCL5, CCR2, CXCL10, Gata3, Rorc, and any combination thereof in one or more cells of an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to expression of one or more inflammatory mediators, such as IL-1β, TNF-α, YM-1, CD86, CCL2, CCL3, CCL5, CCR2, CXCL10, Gata3, Rorc, and any combination thereof in one or more cells of a corresponding individual that is not treated with the anti-TREM2 antibody. In other embodiments, an anti-TREM2 antibody of the present disclosure increases expression of one or more inflammatory mediators, such as IL-1β, TNF-α, YM-1, CD86, CCL2, CCL3, CCL5, CCR2, CXCL10, Gata3, Rorc, and any combination thereof in one or more cells of an individual by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to expression of one or more inflammatory mediators, such as IL-1β, TNF-α, YM-1, CD86, CCL2, CCL3, CCL5, CCR2, CXCL10, Gata3, Rorc, and any combination thereof in one or more cells of a corresponding individual that is not treated with the anti-TREM2 antibody.

In some embodiments, an anti-TREM2 antibody of the present disclosure may decreases expression of one or more inflammatory mediators, such as FLT1, OPN, CSF-1, CD11c, AXL, and any combination thereof in one or more cells of an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to expression of one or more inflammatory mediators, such as FLT1, OPN, CSF-1, CD11c, AXL, and any combination thereof in one or more cells of a corresponding individual that is not treated with the anti-TREM2 antibody. In other embodiments, an anti-TREM2 antibody of the present disclosure decreases expression of one or more inflammatory mediators, such as FLT1, OPN, CSF-1, CD11c, AXL, and any combination thereof in one or more cells of an individual by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to expression of one or more inflammatory mediators, such as FLT1, OPN, CSF-1, CD11c, AXL, and any combination thereof in one or more cells of a corresponding individual that is not treated with the anti-TREM2 antibody.

In some embodiments, an anti-TREM2 antibody of the present disclosure may decrease levels of Abeta peptide in one or more cells of an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to levels of Abeta peptide in one or more cells of a corresponding individual that is not treated with the anti-TREM2 antibody. In other embodiments, an anti-TREM2 antibody of the present disclosure decreases levels of Abeta peptide in one or more cells of an individual by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to levels of Abeta peptide in one or more cells of a corresponding individual that is not treated with the anti-TREM2 antibody.

In some embodiments, an anti-TREM2 antibody of the present disclosure may increase memory of an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the memory of a corresponding individual that is not treated with the anti-TREM2 antibody. In other embodiments, an anti-TREM2 antibody of the present disclosure increases memory of an individual by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the memory of a corresponding individual that is not treated with the anti-TREM2 antibody.

In some embodiments, an anti-TREM2 antibody of the present disclosure may reduce cognitive deficit in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to cognitive deficit in a corresponding individual that is not treated with the anti-TREM2 antibody. In other embodiments, an anti-TREM2 antibody of the present disclosure reduces cognitive deficit an individual by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to cognitive deficit in a corresponding individual that is not treated with the anti-TREM2 antibody.

In some embodiments, an anti-TREM2 antibody of the present disclosure may increase motor coordination in an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to motor coordination in a corresponding individual that is not treated with the anti-TREM2 antibody. In other embodiments, an anti-TREM2 antibody of the present disclosure increases motor coordination an individual by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to motor coordination in a corresponding individual that is not treated with the anti-TREM2 antibody.

Other aspects of the present disclosure relate to methods of enhancing one or more TREM2 activities induced by binding of one or more TREM2 ligands to a TREM2 protein in an individual in need thereof, by administering to the individual a therapeutically effective amount of an anti-TREM2 antibody of the present disclosure. Other aspects of the present disclosure relate to methods of inducing one or more TREM2 activities in an individual in need thereof, by administering to the individual a therapeutically effective amount of an anti-TREM2 antibody of the present disclosure. Any suitable method for measuring TREM2 activity, such as the in vitro cell-based assays or in vivo models of the present disclosure may be used. Exemplary TREM2 activities include, without limitation, TREM2 binding to DAP12; TREM2 phosphorylation; DAP12 phosphorylation; activation of one or more tyrosine kinases, optionally where the one or more tyrosine kinases comprise a Syk kinase, ZAP70 kinase, or both; activation of phosphatidylinositol 3-kinase (PI3K); activation of protein kinase B (Akt); recruitment of phospholipase C-gamma (PLC-gamma) to a cellular plasma membrane, activation of PLC-gamma, or both; recruitment of TEC-family kinase dVav to a cellular plasma membrane; activation of nuclear factor-rB (NF-rB); inhibition of MAPK signaling; phosphorylation of linker for activation of T cells (LAT), linker for activation of B cells (LAB), or both; activation of IL-2-induced tyrosine kinase (Itk); transient activation followed by inhibition of one or more pro-inflammatory mediators selected from IFN-α4, IFN-b, TNF-α, IL-10, IL-6, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, IL-23, CXCL10, VEGF, CCL4, and MCP-1, optionally where the transient activation followed by inhibition occur in one or more cells selected from macrophages, M1 macrophages, activated M1 macrophages, M2 macrophages, dendritic cells, monocytes, osteoclasts, Langerhans cells of skin, Kupffer cells, and microglial cells; phosphorylation of extracellular signal-regulated kinase (ERK); increased expression of C-C chemokine receptor 7 (CCR7) in one or more cells selected from macrophages, M1 macrophages, activated M1 macrophages, M2 macrophages, dendritic cells, monocytes, osteoclasts, Langerhans cells of skin, Kupffer cells, microglia, M1 microglia, activated M1 microglia, and M2 microglia, and any combination thereof; induction of microglial cell chemotaxis toward CCL19 and CCL21 expressing cells; normalization of disrupted TREM2/DAP12-dependent gene expression; recruitment of Syk, ZAP70, or both to a DAP12/TREM2 complex; increasing activity of one or more TREM2-dependent genes, optionally where the one or more TREM2-dependent genes comprise nuclear factor of activated T-cells (NFAT) transcription factors; increased maturation of dendritic cells, monocytes, microglia, M1 microglia, activated M1 microglia, and M2 microglia, macrophages, M1 macrophages, activated M1 macrophages, M2 macrophages, or any combination thereof; increased ability of dendritic cells, monocytes, microglia, M1 microglia, activated M1 microglia, and M2 microglia, macrophages, M1 macrophages, activated M1 macrophages, M2 macrophages, or any combination thereof to induce T-cell proliferation; enhanced ability, normalized ability, or both of bone marrow-derived dendritic cells to induce antigen-specific T-cell proliferation; induction of osteoclast production, increased rate of osteoclastogenesis, or both; increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; increasing the function of dendritic cells, macrophages, M1 macrophages, activated M1 macrophages, M2 macrophages, microglia, M1 microglia, activated M1 microglia, and M2 microglia, or any combination thereof; modulating phagocytosis by dendritic cells, macrophages, M1 macrophages, activated M1 macrophages, M2 macrophages, monocytes, microglia, M1 microglia, activated M1 microglia, and M2 microglia, or any combination thereof; induction of one or more types of clearance selected from apoptotic neuron clearance, nerve tissue debris clearance, non-nerve tissue debris clearance, bacteria or other foreign body clearance, disease-causing agent clearance, tumor cell clearance, or any combination thereof, optionally where the disease-causing agent is selected from amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products including DiPeptide Repeats, (DPRs peptides) composed of glycine-alanine (GA), glycine-proline (GP), glycine-arginine (GR), proline-alanine (PA), or proline-arginine (PR), antisense GGCCCC (G2C4) repeat-expansion RNA; induction of phagocytosis of one or more of apoptotic neurons, nerve tissue debris, non-nerve tissue debris, bacteria, other foreign bodies, disease-causing agents, tumor cells, or any combination thereof, optionally where the disease-causing agent is selected from amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, and Repeat-associated non-ATG (RAN) translation products including DiPeptide Repeats, (DPRs peptides) composed of glycine-alanine (GA), glycine-proline (GP), glycine-arginine (GR), proline-alanine (PA), or proline-arginine (PR), antisense GGCCCC (G2C4) repeat-expansion RNA; increased expression of one or more stimulatory molecules selected from CD83, CD86 MHC class II, CD40, and any combination thereof, optionally where the CD40 is expressed on dendritic cells, monocytes, macrophages, or any combination thereof, and optionally where the dendritic cells comprise bone marrow-derived dendritic cells; reduced secretion of one or more inflammatory mediators, optionally where the one or more inflammatory mediators are selected from CD86, IFN-a4, IFN-b, IL-1β, TNF-α, IL-10, IL-6, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, IL-23, CXCL10, VEGF, CCL4, and MCP-1, and any combination thereof; increased memory; and reduced cognitive deficit.

As disclosed herein, anti-TREM2 antibodies of the present disclosure may be used for decreasing cellular levels of TREM2 on one or more cells, including without limitation, dendritic cells, bone marrow-derived dendritic cells, monocytes, microglia, macrophages, neutrophils, NK cells, osteoclasts, Langerhans cells of skin, and Kupffer cells and/or cell lines. In some embodiments, the present disclosure provides methods of decreasing cellular levels of TREM2 on one or more cells in an individual in need thereof, by administering to the individual a therapeutically effective amount of an anti-TREM2 antibody of the present disclosure. In some embodiments, the one or more cells are selected from dendritic cells, bone marrow-derived dendritic cells, monocytes, microglia, macrophages, neutrophils, NK cells, osteoclasts, Langerhans cells of skin, and Kupffer cells, and any combination thereof. Cellular levels of TREM2 may refer to, without limitation, cell surface levels of TREM2, intracellular levels of TREM2, and total levels of TREM2. In some embodiments, a decrease in cellular levels of TREM2 comprises decrease in cell surface levels of TREM2. As used herein, cell surface levels of TREM2 may be measured by any in vitro cell-based assays or suitable in vivo model described herein or known in the art. In some embodiments, a decrease in cellular levels of TREM2 comprises a decrease in intracellular levels of TREM2. As used herein, intracellular levels of TREM2 may be measured by any in vitro cell-based assays or suitable in vivo model described herein or known in the art. In some embodiments, a decrease in cellular levels of TREM2 comprises a decrease in total levels of TREM2. As used herein, total levels of TREM2 may be measured by any in vitro cell-based assays or suitable in vivo model described herein or known in the art. In some embodiments, the anti-TREM2 antibodies induce TREM2 degradation, TREM2 cleavage, TREM2 internalization, TREM2 shedding, and/or downregulation of TREM2 expression. In some embodiments, cellular levels of TREM2 are measured on primary cells (e.g., dendritic cells, bone marrow-derived dendritic cells, monocytes, microglia, and macrophages) or on cell lines utilizing an in vitro cell assay.

As disclosed herein, anti-TREM2 antibodies of the present disclosure may also be used for increasing memory and/or reducing cognitive deficit. In some embodiments, the present disclosure provides methods of increasing memory and/or reducing cognitive deficit in an individual in need thereof, by administering to the individual a therapeutically effective amount of an anti-TREM2 antibody of the present disclosure.

In certain embodiments, the individual has a heterozygous TREM2 variant allele having an glutamic acid to stop codon substitution in the nucleic acid sequence encoding amino acid residue 14 of the human TREM2 protein (SEQ ID NO: 1). In certain embodiments, the individual has a heterozygous TREM2 variant allele having a glutamine to stop codon substitution in the nucleic acid sequence encoding amino acid residue 33 of the human TREM2 protein (SEQ ID NO: 1). In certain embodiments, the individual has a heterozygous TREM2 variant allele having a tryptophan to stop codon substitution in the nucleic acid sequence encoding amino acid residue 44 of the human TREM2 protein (SEQ ID NO: 1). In certain embodiments, the individual has a heterozygous TREM2 variant allele having an arginine to histidine amino acid substitution at amino acid residue 47 of the human TREM2 protein (SEQ ID NO: 1). In certain embodiments, the individual has a heterozygous TREM2 variant allele having a tryptophan to stop codon substitution in the nucleic acid sequence encoding amino acid residue 78 of the human TREM2 protein (SEQ ID NO: 1). In certain embodiments, the individual has a heterozygous TREM2 variant allele having a valine to glycine amino acid substitution at an amino acid corresponding to amino acid residue 126 of the human TREM2 protein (SEQ ID NO: 1). In certain embodiments, the individual has a heterozygous TREM2 variant allele having an aspartic acid to glycine amino acid substitution at an amino acid corresponding to amino acid residue 134 of the human TREM2 protein (SEQ ID NO: 1). In certain embodiments, the individual has a heterozygous TREM2 variant allele having a lysine to asparagine amino acid substitution at an amino acid corresponding to amino acid residue 186 of the human TREM2 protein (SEQ ID NO: 1).

In some embodiments, the individual has a heterozygous TREM2 variant allele having a guanine nucleotide deletion at a nucleotide corresponding to nucleotide residue G313 of the nucleic acid sequence encoding SEQ ID NO: 1; a guanine nucleotide deletion at a nucleotide corresponding to nucleotide residue G267 of the nucleic acid sequence encoding SEQ ID NO: 1; a threonine to methionine amino acid substitution at an amino acid corresponding to amino acid residue Thr66 of SEQ ID NO: 1; and/or a serine to cysteine amino acid substitution at an amino acid corresponding to amino acid residue Ser116 of SEQ ID NO: 1.

In some embodiments, the individual has a heterozygous DAP12 variant allele having a methionine to threonine substitution at an amino acid corresponding to amino acid residue Mal of SEQ ID NO: 887, a glycine to arginine amino acid substitution at an amino acid corresponding to amino acid residue Gly49 of SEQ ID NO: 887, a deletion within exons 1-4 of the nucleic acid sequence encoding SEQ ID NO: 887, an insertion of 14 amino acid residues at exon 3 of the nucleic acid sequence encoding SEQ ID NO: 887, and/or a guanine nucleotide deletion at a nucleotide corresponding to nucleotide residue G141 of the nucleic acid sequence encoding SEQ ID NO: 887.

As disclosed herein, anti-TREM2 antibodies of the present disclosure may also be used for inducing and/or promoting innate immune cell survival. In some embodiments, the present disclosure provides methods of inducing or promoting innate immune cell survival in an individual in need thereof, by administering to the individual a therapeutically effective amount of an agonist anti-TREM2 antibody of the present disclosure.

As disclosed herein, anti-TREM2 antibodies of the present disclosure may also be used for inducing and/or promoting wound healing, such as after injury. In some embodiments, the wound healing may be colonic wound repair following injury. In some embodiments, the present disclosure provides methods of inducing or promoting wound healing an individual in need thereof, by administering to the individual a therapeutically effective amount of an agonist anti-TREM2 antibody of the present disclosure.

In some embodiments, the methods of the present disclosure may involve the coadministration of anti-TREM2 antibodies, or bispecific antibodies with TLR antagonists or with agents neutralizing TLR agonist (e.g., neutralizing cytokine or interleukin antibodies).

In some embodiments, the methods of the present disclosure may involve the administration of chimeric constructs, including an anti-TREM2 antibody of the present disclosure in conjunction with a TREM2 ligand, such as HSP60.

In some embodiments, the anti-TREM2 antibodies of the present disclosure do not inhibit the growth of one or more innate immune cells. In some embodiments, the anti-TREM2 antibodies of the present disclosure bind to one or more primary immune cells with a K_{D} of less than 50 nM, less than 45 nM, less than 40 nM, less than 35 nM, less than 30 nM, less than 25 nM, less than 20 nM, less than 15 nM, less than 10 nM, less than 9 nM, less than 8 nM, less than 7 nM, less than 6 nM, less than 5 nM, less than 4 nM, less than 3 nM, less than 2 nM, or less than 1 nM. In some embodiments, an anti-TREM2 antibody of the present disclosure accumulates in the brain, or the cerebrospinal fluid (CSF), or both to an extent that is 1% or more, 2% or more, 3% or more, 4% or more, 5% or more, 6% or more, 7% or more, 8% or more, 9% or more, 10% or more of the concentration of the antibody in the blood.

In some embodiments, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

Dementia

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include, without limitation, frontotemporal dementia, Alzheimer's disease, vascular dementia, semantic dementia, and dementia with Lewy bodies.

In some embodiments, administering an anti-TREM2 antibody of the present disclosure can prevent, reduce the risk, and/or treat dementia. hi some embodiments, administering an anti-TREM2 antibody may induce one or more TREM2 activities in an individual having dementia (e.g., DAP12 phosphorylation, PI3K activation, increased expression of one or more anti-inflammatory mediators, or reduced expression of one or more pro-inflammatory mediators).

Frontotemporal Dementia

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)). A majority of those FTD cases with Ub inclusions (FTD-U) were shown to carry mutations in the progranulin gene.

In some embodiments, administering an anti-TREM2 antibody of the present disclosure can prevent, reduce the risk, and/or treat FTD. In some embodiments, administering an anti-TREM2 antibody may induce one or more TREM2 activities in an individual having FTD (e.g., DAP12 phosphorylation, PI3K activation, increased expression of one or more anti-inflammatory mediators, or reduced expression of one or more pro-inflammatory mediators).

Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

In some embodiments, administering an anti-TREM2 antibody of the present disclosure can prevent, reduce the risk, and/or treat Alzheimer's disease. In some embodiments, administering an anti-TREM2 antibody may induce one or more TREM2 activities in an individual having Alzheimer's disease (e.g., DAP12 phosphorylation, PI3K activation, increased expression of one or more anti-inflammatory mediators, or reduced expression of one or more pro-inflammatory mediators).

Nasu-Hakola Disease

Nasu-Hakola disease (NHD), which may alternatively be referred to as polycystic lipomembranous osteodysplasia with sclerosing leukoencephalopathy (PLOSL), is a rare inherited leukodystrophy characterized by progressive presenile dementia associated with recurrent bone fractures due to polycystic osseous lesions of the lower and upper extremities. NHD disease course is generally divided into four stages: latent, osseous, early neurologic, and late neurologic. After a normal development during childhood (latent stage), NHD starts manifesting during adolescence or young adulthood (typical age of onset 20-30 years) with pain in the hands, wrists, ankles, and feet. Patients then start suffering from recurrent bone fractures due to polycystic osseous and osteroporotic lesions in the limb bones (osseous stage). During the third or fourth decade of life (early neurologic stage), patients present with pronounced personality changes (e.g., euphoria, lack of concentration, loss of judgment, and social inhibitions) characteristic of a frontal lobe syndrome. Patients also typically suffer from progressive memory disturbances. Epileptic seizures are also frequently observed. Finally (late neurologic stage), patients progress to a profound dementia, are unable to speak and move, and usually die by the age of 50.

In some embodiments, administering an anti-TREM2 antibody of the present disclosure can prevent, reduce the risk, and/or treat Nasu-Hakola disease (NHD). In some embodiments, administering an anti-TREM2 antibody may induce one or more TREM2 activities in an individual having NHD (e.g., DAP12 phosphorylation, PI3K activation, increased expression of one or more anti-inflammatory mediators, or reduced expression of one or more pro-inflammatory mediators).

Parkinson's Disease

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include, without limitation, tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

In some embodiments, administering an anti-TREM2 antibody of the present disclosure can prevent, reduce the risk, and/or treat Parkinson's disease. In some embodiments, administering an anti-TREM2 antibody may induce one or more TREM2 activities in an individual having Parkinson's disease (e.g., DAP12 phosphorylation, PI3K activation, increased expression of one or more anti-inflammatory mediators, or reduced expression of one or more pro-inflammatory mediators).

Amyotrophic Lateral Sclerosis

As used herein, amyotrophic lateral sclerosis (ALS) or, motor neuron disease or, Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

It has been shown that progranulin play a role in ALS (Schymick, J C et al., (2007) J Neurol Neurosurg Psychiatry.; 78:754-6) and protects again the damage caused by ALS causing proteins such as TDP-43 (Laird, A S et al., (2010). PLoS ONE 5: e13368). It was also demonstrated that pro-NGF induces p75 mediated death of oligodendrocytes and corticospinal neurons following spinal cord injury (Beatty et al., Neuron (2002), 36, pp. 375-386; Giehl et al, Proc. Natl. Acad. Sci USA (2004), 101, pp 6226-30).

In some embodiments, administering an anti-TREM2 antibody of the present disclosure can prevent, reduce the risk, and/or treat ALS. In some embodiments, administering an anti-TREM2 antibody may induce one or more TREM2 activities in an individual having ALS (e.g., DAP12 phosphorylation, PI3K activation, increased expression of one or more anti-inflammatory mediators, or reduced expression of one or more pro-inflammatory mediators).

Huntington's Disease

Huntington's disease (HD) is an inherited neurodegenerative disease caused by an autosomal dominant mutation in the Huntingtin gene (HTT). Expansion of a cytokine-adenine-guanine (CAG) triplet repeat within the Huntingtin gene results in production of a mutant form of the Huntingtin protein (Htt) encoded by the gene. This mutant Huntingtin protein (mHtt) is toxic and contributes to neuronal death. Symptoms of Huntington's disease most commonly appear between the ages of 35 and 44, although they can appear at any age.

Symptoms of Huntington's disease, include, without limitation, motor control problems, jerky, random movements (chorea), abnormal eye movements, impaired balance, seizures, difficulty chewing, difficulty swallowing, cognitive problems, altered speech, memory deficits, thinking difficulties, insomnia, fatigue, dementia, changes in personality, depression, anxiety, and compulsive behavior.

In some embodiments, administering an anti-TREM2 antibody of the present disclosure can prevent, reduce the risk, and/or treat Huntington's disease (HD). In some embodiments, administering an anti-TREM2 antibody may induce one or more TREM2 activities in an individual having HD (e.g., DAP12 phosphorylation, PI3K activation, increased expression of one or more anti-inflammatory mediators, or reduced expression of one or more pro-inflammatory mediators).

Tauopathy Disease

Tauopathy diseases, or Tauopathies, are a class of neurodegenerative disease caused by aggregation of the microtubule-associated protein tau within the brain. Alzheimer's disease (AD) is the most well-known tauopathy disease, and involves an accumulation of tau protein within neurons in the form of insoluble neurofibrillary tangles (NFTs). Other tauopathy diseases and disorders include progressive supranuclear palsy, dementia pugilistica (chromic traumatic encephalopathy), Frontotemporal dementia and parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, frontotemporal dementia, and frontotemporal lobar degeneration.

In some embodiments, administering an anti-TREM2 antibody of the present disclosure can prevent, reduce the risk, and/or treat tauopathy disease. In some embodiments, administering an anti-TREM2 antibody may induce one or more TREM2 activities in an individual having tauopathy disease (e.g., DAP12 phosphorylation, PI3K activation, increased expression of one or more anti-inflammatory mediators, or reduced expression of one or more pro-inflammatory mediators).

Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

In some embodiments, administering an anti-TREM2 antibody of the present disclosure can prevent, reduce the risk, and/or treat multiple sclerosis. In some embodiments, administering an anti-TREM2 antibody may induce one or more TREM2 activities in an individual having multiple sclerosis (e.g., DAP12 phosphorylation, PI3K activation, increased expression of one or more anti-inflammatory mediators, and reduced expression of one or more pro-inflammatory mediators).

Cancer

Yet further aspects of the present disclosure provide methods for preventing, reducing risk, or treating an individual having cancer, comprising administering to the individual a therapeutically effective amount of an isolated anti-TREM2 antibody of the present disclosure. Any of the isolated antibodies of the present disclosure may be used in these methods. In some embodiments, the isolated antibody is an agonist antibody of the present disclosure. In other embodiments, the isolated antibody is an antagonist antibody of the present disclosure.

As described above, the tumor microenvironment is known to contain a heterogeneous immune infiltrate, which includes T lymphocytes, macrophages and cells of myeloid/granulocytic lineage. In particular, the presence of M2-macrophages in tumors is associated with poor prognosis. Therapies that reduce the number of these cells in the tumor, such as CSF-1R blocking agents, are showing beneficial effects in preclinical models and early stage clinical studies. It has been shown that TREM2 synergizes with CSF-1 to promote survival of macrophages in vitro, and that this effect is particularly prominent in M2-type macrophages, compared to other types of phagocytic cells. A seminal preclinical study has also shown synergies between drugs that target tumor-associated macrophages (e.g., CSF-1/CSF-1R blocking antibodies) and checkpoint blocking antibodies that target T cells, indicating that manipulating both cell types shows efficacy in tumor models where individual therapies are poorly effective (Zhu Y; Cancer Res. 2014 Sep. 15; 74(18):5057-69). Therefore, without wishing to be bound by theory, it is thought that blocking TREM2 signaling in tumor associated macrophages may inhibit suppression of the immune response in the tumor microenvironment, resulting in a therapeutic anti-tumor immune response.

Due to the synergies between TREM2 and CSF-1, and between targeting tumor-associated macrophages and targeting T cells, in some embodiments, the methods for preventing, reducing risk, or treating an individual having cancer further include administering to the individual at least one antibody that specifically binds to an inhibitory checkpoint molecule. Examples of antibodies that specifically bind to an inhibitory checkpoint molecule include, without limitation, an anti-PD-L1 antibody, an anti-CTLA-4 antibody, an anti-PD-L2 antibody, an anti-PD-1 antibody, an anti-B7-H3 antibody, an anti-B7-H4 antibody, and anti-HVEM antibody, an anti-BTLA antibody, an anti-GALS antibody, an anti-TIM3 antibody, an anti-AZAR antibody, an anti-LAG-3 antibody, an anti-phosphatidylserine antibody, and any combination thereof. In some embodiments, the at least one antibody that specifically binds to an inhibitory checkpoint molecule is administered in combination with an antagonist anti-TREM2 antibody of the present disclosure.

In some embodiments, a cancer to be prevented or treated by the methods of the present disclosure includes, but is not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma; chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In some embodiments, the cancer is colorectal cancer. In some embodiments, the cancer is selected from non-small cell lung cancer, glioblastoma, neuroblastoma, renal cell carcinoma, bladder cancer, ovarian cancer, melanoma, breast carcinoma, gastric cancer, and hepatocellular carcinoma. In some embodiments, the cancer is triple-negative breast carcinoma. In some embodiments, the cancer may be an early stage cancer or a late stage cancer. In some embodiments, the cancer may be a primary tumor. In some embodiments, the cancer may be a metastatic tumor at a second site derived from any of the above types of cancer.

In some embodiments, anti-TREM2 antibodies of the present disclosure may be used for preventing, reducing risk, or treating cancer, including, without limitation, bladder cancer breast cancer, colon and rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, and thyroid cancer.

In some embodiments, the present disclosure provides methods of preventing, reducing risk, or treating an individual having cancer, by administering to the individual a therapeutically effective amount of an anti-TREM2 antibody of the present disclosure.

In some embodiments, the method further includes administering to the individual at least one antibody that specifically binds to an inhibitory checkpoint molecule, and/or another standard or investigational anti-cancer therapy. In some embodiments, the at least one antibody that specifically binds to an inhibitory checkpoint molecule is administered in combination with the isolated antibody. In some embodiments, the at least one antibody that specifically binds to an inhibitory checkpoint molecule is selected from an anti-PD-L1 antibody, an anti-CTLA-4 antibody, an anti-PD-L2 antibody, an anti-PD-1 antibody, an anti-B7-H3 antibody, an anti-B7-H4 antibody, and anti-HVEM antibody, an anti-B- and T-lymphocyte attenuator (BTLA) antibody, an anti-Killer inhibitory receptor (KIR) antibody, an anti-GALS antibody, an anti-TIM3 antibody, an anti-AZAR antibody, an anti-LAG-3 antibody, an anti-phosphatidylserine antibody, an anti-CD27 antibody, and any combination thereof. In some embodiments, the standard or investigational anti-cancer therapy is one or more therapies selected from radiotherapy, cytotoxic chemotherapy, targeted therapy, imatinib (Gleevec®), trastuzumab (Herceptin®), adoptive cell transfer (ACT), chimeric antigen receptor T cell transfer (CAR-T), vaccine therapy, hormonal therapy, bevacizumab (Avastin®), Ofatumumab (Arzerra®), Rituximab (Rituxan®, MabThera®, Zytux®), cryotherapy, ablation, radiofrequency ablation, and cytokine therapy.

In some embodiments, the method further includes administering to the individual at least one antibody that specifically binds to an inhibitory cytokine. In some embodiments, the at least one antibody that specifically binds to an inhibitory cytokine is administered in combination with the isolated antibody. In some embodiments, the at least one antibody that specifically binds to an inhibitory cytokine is selected from an anti-CCL2 antibody, an anti-CSF-1 antibody, an anti-IL-2 antibody, and any combination thereof.

In some embodiments, the method further includes administering to the individual at least one agonistic antibody that specifically binds to a stimulatory checkpoint protein. In some embodiments, the at least one agonistic antibody that specifically binds to a stimulatory checkpoint protein is administered in combination with the isolated antibody. In some embodiments, the at least one agonistic antibody that specifically binds to a stimulatory checkpoint protein is selected from an agonist anti-CD40 antibody, an agonist anti-0X40 antibody, an agonist anti-ICOS antibody, an agonist anti-CD28 antibody, an agonist anti-CD137/4-1BB antibody, an agonist anti-CD27 antibody, an agonist anti-glucocorticoid-induced TNFR-related protein GITR antibody, and any combination thereof.

In some embodiments, the method further includes administering to the individual at least one stimulatory cytokine. In some embodiments, the at least one stimulatory cytokine is administered in combination with the isolated antibody. In some embodiments, the at least one stimulatory cytokine is selected from TNF-α, IL-1α, IL-1β, IL-10, IL-6, IL-8, CRP, TGF-beta members of the chemokine protein families, IL-20 family members, IL-33, LIF, IFN-gamma, OSM, CNTF, TGF-beta, IL-11, IL-12, IL-17, IL-8, CRP, IFN-α, IFN-β, IL-2, IL-18, IL-23, CXCL10, CCL4, MCP-1, VEGF, GM-CSF, G-CSF, and any combination thereof.

Kits/Articles of Manufacture

The present disclosure also provides kits containing an isolated antibody of the present disclosure (e.g., an anti-TREM2 or anti-DAP12 antibody described herein), or a functional fragment thereof. Kits of the present disclosure may include one or more containers comprising a purified antibody of the present disclosure. In some embodiments, the kits further include instructions for use in accordance with the methods of this disclosure. In some embodiments, these instructions comprise a description of administration of the isolated antibody of the present disclosure (e.g., an anti-TREM2 or anti-DAP12 antibody described herein) to prevent, reduce risk, or treat an individual having a disease, disorder, or injury selected from dementia, frontotemporal dementia, Alzheimer's disease, Nasu-Hakola disease, multiple sclerosis, and cancer, according to any methods of this disclosure.

In some embodiments, the instructions comprise a description of how to detect TREM2 and/or DAP12, for example in an individual, in a tissue sample, or in a cell. The kit may further comprise a description of selecting an individual suitable for treatment based on identifying whether that individual has the disease and the stage of the disease.

In some embodiments, the kits may further include another antibody of the present disclosure (e.g., at least one antibody that specifically binds to an inhibitory checkpoint molecule, at least one antibody that specifically binds to an inhibitory cytokine, and/or at least one agonistic antibody that specifically binds to a stimulatory checkpoint protein) and/or at least one stimulatory cytokine. In some embodiments, the kits may further include instructions for using the antibody and/or stimulatory cytokine in combination with an isolated antibody of the present disclosure (e.g., an anti-TREM2 antagonist antibody described herein), instructions for using the isolated antibody of the present disclosure in combination with an antibody and/or stimulatory cytokine, or instructions for using an isolated antibody of the present disclosure and an antibody and/or stimulatory cytokine, according to any methods of this disclosure.

The instructions generally include information as to dosage, dosing schedule, and route of administration for the intended treatment. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a disease of the present disclosure. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is an isolated antibody of the present disclosure (e.g., an anti-TREM2 or anti-DAP12 antibody described herein). The container may further comprise a second pharmaceutically active agent.

Kits may optionally provide additional components such as buffers and interpretive information. Normally, the kit comprises a container and a label or package insert(s) on or associated with the container.

### Diagnostic Uses

The isolated antibodies of the present disclosure (e.g., an anti-TREM2 or anti-DAP12 antibody described herein) also have diagnostic utility. This disclosure therefore provides for methods of using the antibodies of this disclosure, or functional fragments thereof, for diagnostic purposes, such as the detection of TREM2 and/or DAP12 in an individual or in tissue samples derived from an individual.

In some embodiments, the individual is a human. In some embodiments, the individual is a human patient suffering from, or at risk for developing, cancer. In some embodiments, the diagnostic methods involve detecting TREM2 and/or DAP12 in a biological sample, such as a biopsy specimen, a tissue, or a cell. An isolated antibody of the present disclosure (e.g., an anti-TREM2 or anti-DAP12 antibody described herein) is contacted with the biological sample and antigen-bound antibody is detected. For example, a tumor sample (e.g., a biopsy specimen) may be stained with an anti-TREM2 or anti-DAP12 antibody described herein in order to detect and/or quantify tumor-associated macrophages (e.g., M2-type macrophages). The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain embodiments, the antibody is radiolabeled, for example with ¹⁸F and subsequently detected utilizing micro-positron emission tomography analysis. Antibody-binding may also be quantified in a patient by non-invasive techniques such as positron emission tomography (PET), X-ray computed tomography, single-photon emission computed tomography (SPECT), computed tomography (CT), and computed axial tomography (CAT).

In other embodiments, an isolated antibody of the present disclosure (e.g., an anti-TREM2 or anti-DAP12 antibody described herein) may be used to detect and/or quantify, for example, microglia in a brain specimen taken from a preclinical disease model (e.g., a non-human disease model). As such, an isolated antibody of the present disclosure (e.g., an anti-TREM2 or anti-DAP12 antibody described herein) may be useful in evaluating therapeutic response after treatment in a model for a nervous system disease or injury such as dementia, frontotemporal dementia, Alzheimer's disease, Nasu-Hakola disease, or multiple sclerosis, as compared to a control.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Production, Identification, and Characterization of Agonist Anti-TREM2 Antibodies

### Introduction

The amino acid sequence of the human TREM2 preprotein is set forth below in SEQ ID NO: 1. Human TREM2 contains a signal peptide located at amino residues 1-18 of SEQ ID NO: 1. Human TREM2 contains an extracellular immunoglobulin-like variable-type (IgV) domain located at amino residues 29-112 of SEQ ID NO: 1; additional extracellular sequences located at amino residues 113-174 of SEQ ID NO: 1; a transmembrane domain located at amino residues 175-195 of SEQ ID NO: 1; and an intracellular domain located at amino residues 196-230 of SEQ ID NO: 1.

TREM2 amino acid sequence (SEQ ID NO: 1):

```
                10 20 30 40
MEPLRLLILL FVTELSGAHN TTVFQGVAGQ SLQVSCPYDS
                50 60 70 80
MKHWGRRKAW CRQLGEKGPC QRVVSTHNLW LLSFLRRWNG
                90 100 110 120
STAITDDTLG GTLTITLRNL QPHDAGLYQC QSLHGSEADT
              130 140 150 160
LRKVLVEVLA DPLDHRDAGD LWFPGESESF EDAHVEHSIS
              170 180 190 200
RSLLEGEIPF PPTSILLLLA CIFLIKILAA SALWAAAWHG
              210 220 230
QKPGTHPPSE LDCGHDPGYQ LQTLPGLRDT
```

A known feature of human TREM2 is that the transmembrane domain contains a lysine (aa186) that can interact with an aspartic acid in DAP12, a key adaptor protein that transduces signaling from TREM2, TREM1, and other related IgV family members.

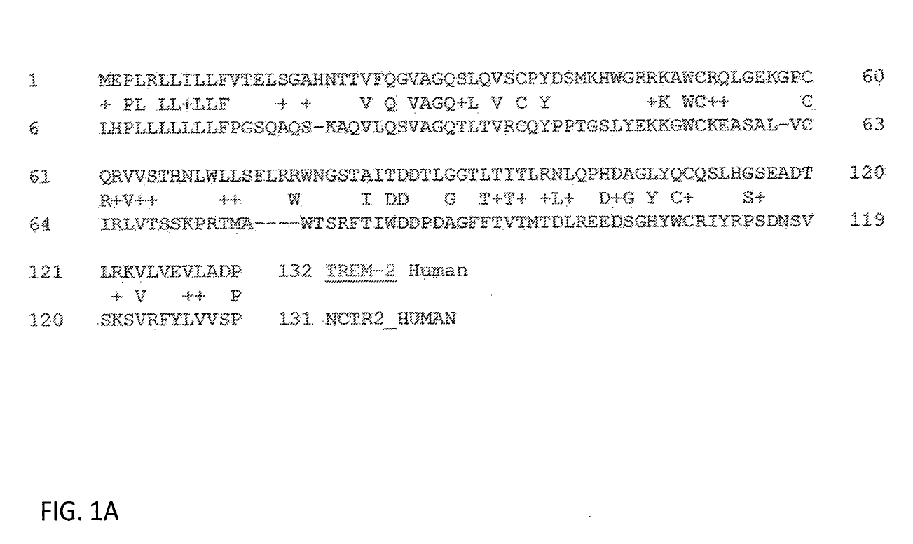

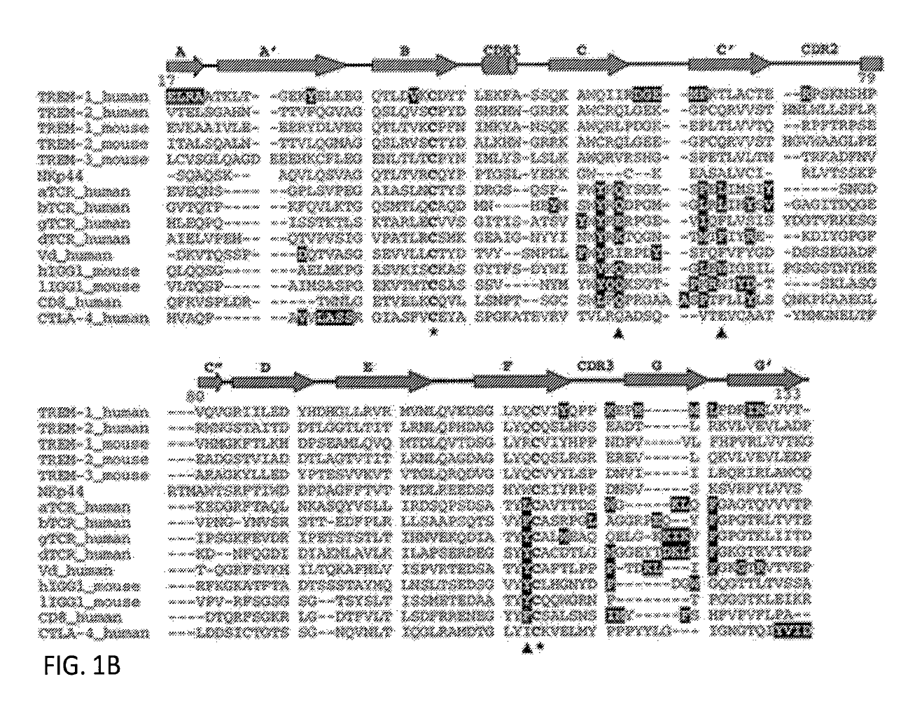

A BLAST analysis of human TREM2 identified 18 related homologues. These homologues included the Natural Killer (NK) cell receptor NK-p44 (NCTR2), the polymeric immunoglobulin receptor (pIgR), CD300E, CD300A, CD300C, and TREML1/TLT1. The closest homologue was identified as NCTR2, having similarity with TREM2 within the IgV domain (FIG. 1A). A BLAST analysis also compared TREM proteins with other IgV family proteins (FIG. 1B).

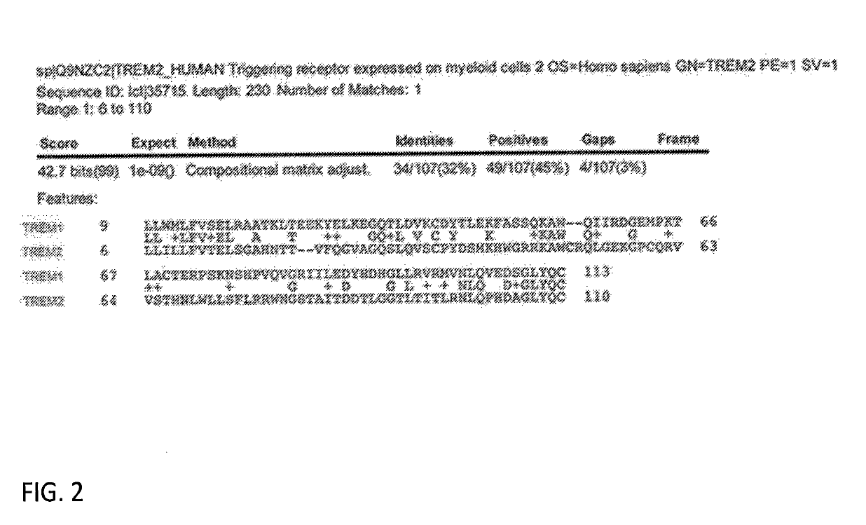

TREM2 is also related to TREM1. An alignment of the amino acid sequences of TREM1 and TREM2 was generated by 2-way blast (FIG. 2). This is limited to the IgV domain as well.

Antibodies that bind the extracellular domain of TREM2, particularly the extra cellular domain (amino acid residues 19-174 of SEQ ID NO: 1) are generated using mouse hybridoma technology, phage display technology, and yeast display technology. Antibodies are then screened for their ability to bind cells that express TREM2 and for their ability to activate TREM2 signaling and functions in cells and in a whole animal in vivo as described in Examples 2-48 below. For example, agonist anti-TREM2 antibodies can be produced that target the IgV domain (amino acid residues 29-112). IgV domains bind to targets, and through multimerization of receptors, lead to activation. Thus these domains are rational targets for agonistic antibodies. They are also highly divergent.

### Results

Anti-TREM2 Antibody Production

Immunization Procedure

Rapid Prime Method:

Four 50-day old female BALB/c mice were immunized with using the following procedure. A series of subcutaneous aqueous injections containing human TREM2 antigen but no adjuvant were given over a period of 19 days. Mice were housed in a ventilated rack system from Lab Products. All four mice were euthanized on Day 19 and lymphocytes were harvested for hybridoma cell line generation.

Standard Method:

Four 50-day old female BALB/c mice, NZB/W mice, or Trem2tml (KOMP)Vlcg mice were immunized using the following procedure. Mice were housed in a ventilated rack system from Lab Products. Mice were injected intraperitoneally every 3 weeks with a human TREM2 antigen mixed in CpG-ODN adjuvant at 25 μg protein antigen per mouse (total volume 125 μL, per mouse). Test bleeds were done by saphenous vein lancing seven days after the second boost. The test bleed (immune sera) was tested by indirect ELISA assay to determine the best two responding mice for the fusion. The mice may require a 3rd and 4th boost and another test bleed 7 days after boost to assess titer before fusion. When the antibody titer is high enough the best two responding mice are given a final intravenous boost via lateral tail vein. Four days after the IV boost the mice were euthanized for fusion. The spleens were harvested and lymphocytes isolated from the spleen were used in the fusion process to produce hybridomas.

HTV Method

Ten female Trem2tml (KOMP)Vlcg mice were immunized using the following procedure according to Bates et al., Biotechniques 2006, 40 (2): 199-208 and Hazen et al., Landes Bioscience 2014, 6:1, 95-107. Mice were housed in a ventilated rack system from Lab Products. Endotoxin free recombinant DNA constructs were produced by BlueSky Technologies. Human Trem2-Dap12 fusion protein was subcloned into the pCAGGS-Kan plasmid and pUNO-mGMCSF and pUNO-mF1t3La plasmids were purchased from Kerafast. Plasmid DNA in PBS was diluted in warm Ringer's solution to 10% of the mice body weight and transferred to a 3 ml syringe with 29G needle. For the hydrodynamic tail vein injection (HTV), mice were lightly anesthesized with Isoflurane on a heat pad and DNA was bolus injected into the lateral tail vein over 6-10 seconds. Mice were allowed to recover for 2 minutes on the heat pad and observed for any acute effects for 10 minutes after injection. Mice were boosted up to five times weekly. Immune response was assessed by test bleeding the mice 5 days post 4^{th} boost using indirect Elisa on Trem2 antigen. Mice with the best IgG titer will be used for hybridoma development.

Hybridoma Development

Lymphocytes were isolated and fused with murine SP2/0 myeloma cells in the presence of poly-ethylene glycol (PEG 1500) as per standard Roche Protocol. Fused cells were cultured using a single-step cloning method (HAT selection). This method uses a semi-solid methylcellulose-based HAT selective medium to combine the hybridoma selection and cloning into one step. Single cell-derived hybridomas grow to form monoclonal colonies on the semi-solid media. Ten days after the fusion event, 948 of the resulting hybridoma clones were transferred to 96-well tissue culture plates and grown in HT containing medium until mid-log growth was reached (5 days).

Hybridoma Screening

Tissue culture supernatants from the 948 hybridomas were tested by indirect ELISA on screening antigen (Primary Screening) and probed for both IgG and IgM antibodies using a Goat anti-IgG/IgM(H&L)-HRP secondary and developed with TMB substrate. Clones >0.2 OD in this assay were taken to the next round of testing. Positive cultures were retested on screening antigen to confirm secretion and on an irrelevant antigen (Human Transferrin) to eliminate non-specific or “sticky” mAbs and rule out false positives. All clones of interest were isotyped by antibody trapping ELISA to determine if they are IgG or IgM isotype.

Hybridoma Cell Culture

The hybridoma cell lines of interest were maintained in culture in 24-well culture plates for 32 days post transfer to 96-well plates. This is referred to as the stability period and tests whether clones remain stable and secreting. During this stability period time temporary frozen cell line back up is made of all the clones of interest for −80° C. storage (viable 6 months). Hybridomas were periodically tested during this time period for secretion and specificity.

Subcloning

The top hybridoma cell lines (clones) were subcloned to ensure monoclonality. Subcloning was performed by plating parental clones out again using the single-step cloning system. Between 24 and 90 subclones were transferred to 96-well culture plates. Subclones were screened by indirect ELISA and antibody trapping ELISA. The top subclones for each parent were taken for expansion in culture. Any parental clones that were <50% clonal had a second round of subcloning performed.

The antibodies were then screened for TREM2 binding. Antibodies that were positive for binding to human TREM2 were tested for ability to block ligand binding and ability to induce, enhance, or otherwise increase ligand-induced TREM2 activity in multiple cell types. The isotype and bin category of each of the antibodies are listed in Table 1. In Table 1, “ND” refers to antibodies for which the Bin category has not been determined.

TABLE 1
Anti-TREM2 antibodies

| Ab ID | Ab Isotype | Bin |
|---|---|---|
| 1A7 | mIgG1 | 3 |
| 3A2 | mIgG1 | 1 |
| 3B10 | mIgG1 | 4 |
| 6G12 | mIgG1 | 1 |
| 6H6 | mIgG2b | 1 |
| 7A9 | mIgG1 | ND |
| 7B3 | mIgG1 | 4 |
| 8A1 | mIgG2a | 3 |
| 8E10 | mIgG1 | 2-3 |
| 8F11 | mIgG1 | 2 |
| 8F8 | mIgG1 | 4 |
| 9F5 | mIgG1 | ND |
| 9G1 | mIgG1 | 4 |
| 9G3 | mIgG1 | 4 |
| 10A9 | mIgG1 | 1 |
| 10C1 | mIgG2b | 1 |
| 11A8 | mIgG1 | 4 |
| 12E2 | mIgG1 | 4 |
| 12F9 | mIgG1 | 4 |
| 12G6 | mIgG1 | ND |
| 2C7 | mIgG1 | 4 |
| 2F5 | mIgG1 | |
| 3C1 | mIgG1 | 4 |
| 4D7 | mIgG1 | 4 |
| 4D11 | mIgG1 | 4 |
| 6C11 | mIgG1 | 4 |
| 6G12 | mIgG1 | ND |
| 7A3 | mIgG1 | 4 |
| 7C5 | mIgG1 | 4 |
| 7E9 | mIgG1 | 4 |
| 7F6 | mIgG1 | 4 |
| 7G1 | mIgG1 | 4 |
| 7H1 | mIgG1 | ND |
| 8C3 | mIgG1 | 4 |
| 8F10 | mIgG1 | ND |
| 12A1 | mIgG1 | 4 |
| 1E9 | mIgG2b | 4 |
| 2C5 | mIgG2b | 1/4 |
| 3C5 | mIgG2b | 4 |
| 4C12 | mIgG2b | 4 |
| 4F2 | mIgG2b | 4 |
| 5A2 | mIgG2a | 4 |
| 6B3 | mIgG2a | 1 |
| 7D1 | mIgG2a | 4 |
| 7D9 | mIgG2b | 4 |
| 11D8 | mIgG2b | 4 |
| 8A12 | mIgG1 | 3 |
| 10E7 | mIgG1 | ND |
| 10B11 | mIgG2a | ND |
| 10D2 | mIgG2b | ND |
| 7D5 | mIgG2a | ND |
| 2A7 | mIgG2a | ND |
| 3G12 | mIgG1 | ND |
| 6H9 | mIgG2a | ND |
| 8G9 | mIgG2a | ND |
| 9B4 | mIgG2a | ND |
| 10A1 | mIgG1 | ND |
| 11A8 | mIgG2a | ND |
| 12F3 | mIgG2a | ND |
| 1H7 | mIgG1 | 1 |
| 2F6 | mIgG1 | 4 |
| 2H8 | mIgG1 | 1 |
| 3A7 | mIgG1 | 4 |
| 7E5 | mIgG1 | 4 |
| 7F8 | mIgG1 | 1 |
| 11H5 | mIgG1 | 4 |
| 1B4 | mIgG2a | 4 |
| 6H2 | mIgG2a | ND |
| 7B11 | mIgG2a | 4 |
| 18D8 | mIgG2a | 1 |
| 18E4 | mIgG2a | ND |
| 29F6 | mIgG1 | 4 |
| 40D5 | mIgG2a | ND |
| 43B9 | mIgG2a | 4 |
| 44A8 | mIgG2a | ND |
| 44B4 | mIgG1 | ND |
| 45D6 | mIgG2a | 4 |
| 29F7 | mIgG2a | ND |
| 32G1 | mIgG2a | 4 |

Antibody Heavy Chain and Light Chain Variable Domain Sequences

Using standard techniques, the amino acid sequences encoding the light chain variable and the heavy chain variable domains of the generated antibodies were determined. The EU or Kabat light chain HVR sequences of the antibodies are set forth in Table 2A. The EU or Kabat light chain HVR consensus sequences of the antibodies are set forth in Table 2B. The EU or Kabat heavy chain HVR sequences of the antibodies are set forth in Table 3A. The EU or Kabat heavy chain HVR consensus sequences of the antibodies are set forth in Table 3B. The EU or Kabat light chain framework (FR) sequences of the antibodies are set forth in Table 4A. The EU or Kabat heavy chain framework (FR) sequences of the antibodies are set forth in Table 4B. The EU or Kabat heavy chain HVR

TABLE 2A
EU or Kabat light chain HVR sequences

| Ab | HVR L1 | HVR L2 | HVR L3 |
|---|---|---|---|
| 4D11 | RASENIYSFLA (SEQ ID | NSKTFAE (SEQ ID | QHHYGTPPWT (SEQ ID NO: 34) |
| | NO: 9) | NO: 24) | |
| 78C5 | RASENIYSFLA (SEQ ID | NSKTFAE (SEQ ID | QHHYGTPPWT (SEQ ID NO: 34) |
| | NO: 9) | NO: 24) | |
| 6G12 | KSSQSLLYSSNQKNCLA | WAFTRES (SEQ ID | QQYYSYPLT (SEQ ID NO: 35) |
| | (SEQ ID NO: 10) | NO: 25) | |
| 8F11 | KSSQSLLYSNGKTFLS (SEQ | LVSKLDS (SEQ ID | MQGTHFPLT (SEQ ID NO: 36) |
| | ID NO: 11) | NO: 26) | |
| 8E10 | KSSQSLLDSDGKTYLN | LVSKLDS (SEQ ID | WQGTHFPYT (SEQ ID NO: 37) |
| | (SEQ ID NO: 12) | NO: 26) | |
| 7E5 | KSSQSLLYSNGKTFLS (SEQ | LVSKLDS (SEQ ID | MQGTHFPLT (SEQ ID NO: 36) |
| | ID NO: 11) | NO: 26) | |
| 7F8 | SASSSVSYMY (SEQ ID | LTSILAS (SEQ ID NO: 27) | QQWSFNPYT (SEQ ID NO: 38) |
| | NO: 13) | | |
| 8F8 | RSSQSLVHSNGNTYLH | KVSNRFS (SEQ ID | SQSTHVPLT (SEQ ID NO: 39) |
| | (SEQ ID NO: 14) | NO: 28) | |
| 1H7 | SASSSVSYMY (SEQ ID | LTSILAS (SEQ ID NO: 27) | QQWSFNPYT (SEQ ID NO: 38) |
| | NO: 13) | | |
| 2H8 | SASSSVSYMY (SEQ ID | LTSILAS (SEQ ID NO: 27) | QQWSFNPYT (SEQ ID NO: 38) |
| | NO: 13) | | |
| 3A2 | RSSQTIIHSNGNTYLE (SEQ | KVSNRFS (SEQ ID | FQGSHVPYT (SEQ ID NO: 40) |
| | ID NO: 15) | NO: 28) | |
| 3A7 | KSSQSLLYSNGKTFLS (SEQ | LVSKLDS (SEQ ID | MQGTHFPLT (SEQ ID NO: 36) |
| | ID NO: 11) | NO: 26) | |
| 3B10 | KSSQSLLYSSDQKNYLA | WASTRES (SEQ ID | QQYYSYPLT (SEQ ID NO: 35) |
| | (SEQ ID NO: 16) | NO: 29) | |
| 4F11 | RSSQTIIHSNGNTYLE (SEQ | KVSNRFS (SEQ ID | FQGSHVPYT (SEQ ID NO: 40) |
| | ID NO: 15) | NO: 28) | |
| 6H6 | KSSQSVFYSSNQKNYLA | WASTRES (SEQ ID | HQYLSSLT (SEQ ID NO: 582) |
| | (SEQ ID NO: 581) | NO: 29) | |
| 7A9 | RASENIYSYLA (SEQ ID | KAKTLAE (SEQ ID | QHHYGTPFT (SEQ ID NO: 41) |
| | NO: 17) | NO: 30) | |
| 8A1 | RTSENVYSNLA (SEQ ID | AATNLAD (SEQ ID | HHFWGTPYT (SEQ ID NO: 42) |
| | NO: 18) | NO: 31) | |
| 9E5 | RSSQSLVHSNGYTYLH | KVSNRFS (SEQ ID | SQSTRVPYT (SEQ ID NO: 43) |
| | (SEQ ID NO: 19) | NO: 28) | |
| 9G1 | RFSQSLVHSNGNTYLH | KVSNRFS (SEQ ID | SQSTRVPPT (SEQ ID NO: 44) |
| | (SEQ ID NO: 20) | NO: 28) | |
| 9G3 | KASSNVNYMS (SEQ ID | FTSNLPS (SEQ ID NO: 32) | SGEVTQFT (SEQ ID NO: 45) |
| | NO: 21) | | |
| 10A9 | RSSQTIIHSNGNTYLE (SEQ | KVSNRFC (SEQ ID | FQGSHVPYT (SEQ ID NO: 40) |
| | ID NO: 15) | NO: 33) | |
| 11A8 | KSSQSLLNSGNQKKYLT | WASTRES (SEQ ID | QNDYGFPLT (SEQ ID NO: 46) |
| | (SEQ ID NO: 22) | NO: 29) | |
| 12D9 | KSSQSLLYSGNQKNFLA | WASTRES (SEQ ID | QQYYSYPFT (SEQ ID NO: 47) |
| | (SEQ ID NO: 23) | NO: 29) | |
| 12E9 | KSSQSLLYSSDQKNYLA | WASTRES (SEQ ID | QQYYSYPLT (SEQ ID NO: 35) |
| | (SEQ ID NO: 16) | NO: 29) | |
| 10C1 | KSSQSVFYSSNQKNYLA | WASTRES (SEQ ID | HQYLSSLT (SEQ ID NO: 582) |
| | (SEQ ID NO: 581 | NO: 29) | |
| 7E9 | KSSQSLLYSSNQKNCLA | WASTRES (SEQ ID | QQYYSYPLT (SEQ ID NO: 35) |
| | (SEQ ID NO: 10) | NO: 29) | |
| 8C3 | RSSQSLVHSNGNTYLH | KVSNRFS (SEQ ID | SQSTHVPPT (SEQ ID NO: 583) |
| | (SEQ ID NO: 14) | NO: 28) | |
| 1B4v1 | SQDVSTTVA (SEQ ID | SASYRYT (SEQ ID | QQHYSTPPT (SEQ ID NO: 698) |
| | NO: 690) | NO: 695) | |
| 1B4v2 | SQSLVHSNGNTYLH (SEQ | KVSNRVS (SEQ ID | SQSTHVPLT (SEQ ID NO: 39) |
| | ID NO: 734) | NO: 739) | |
| 6H2 | SQSIVHSNGNTYLE (SEQ ID | KVSNRFS (SEQ ID | FQGSHVPFT (SEQ ID NO: 699) |
| | NO: 691) | NO: 28) | |
| 7B11 | SQGVSTAVA (SEQ ID | WASTRHT (SEQ ID | HQHYSTYT (SEQ ID NO: 700) |
| | NO: 692) | NO: 696) | |
| 18D8 | SQDVRTAVA (SEQ ID | SASYRYT (SEQ ID | QQHYGTPPWT (SEQ ID |
| | NO: 693) | NO: 695) | NO: 701) |
| 18E4v1 | SENVVTYVS (SEQ ID | GASNRYT (SEQ ID | GQGYSYPYT (SEQ ID NO: 702) |
| | NO: 694) | NO: 697) | |
| 18E4v2 | SQSLVHSNGNTYLH (SEQ | KVSDRFS (SEQ ID | SQSTHVPLT (SEQ ID NO: 39) |
| | ID NO: 734) | NO: 740) | |
| 29F6v1 | SQDVRTAVA (SEQ ID | SASYRYT (SEQ ID | QQHYGTPPWT (SEQ ID |
| | NO: 693) | NO: 695) | NO: 701) |
| 29F6v2 | SQSLVHSNGDTYLH (SEQ | KVSNRFS (SEQ ID | SQSTHVPLT (SEQ ID NO: 39) |
| | ID NO: 735) | NO: 28) | |
| 40D5 | SQDVRTAVA (SEQ ID | SASYRYT (SEQ ID | QQHYGTPPWT (SEQ ID |
| | NO: 693) | NO: 695) | NO: 701) |
| 43B9 | SQDVRTAVA (SEQ ID | SASYRYT (SEQ ID | QQHYGTPPWT (SEQ ID |
| | NO: 693) | NO: 695) | NO: 701) |
| 44A8v1 | SQDVSTTVA (SEQ ID | SASYRYT (SEQ ID | QQHYSTPPT (SEQ ID NO: 698) |
| | NO: 690) | NO: 695) | |
| 44A8v2 | SESVDYHGTSLMQ (SEQ ID | AASNVES (SEQ ID | QQNRKILWT (SEQ ID NO: 744) |
| | NO: 736) | NO: 741) | |
| 44B4v1 | SQDVRTAVA (SEQ ID | SASYRYT (SEQ ID | QQHYGTPPWT (SEQ ID |
| | NO: 693) | NO: 695) | NO: 701) |
| 44B4v2 | SENIZYSLA (SEQ ID | NANSLED (SEQ ID | KQAYDVPWT (SEQ ID NO: 745) |
| | NO: 737) | NO: 742) | |
| 29F7 | RASQSIGTSIH (SEQ ID | FASESIS (SEQ ID NO: 743) | QQTNTWPIT (SEQ ID NO: 746) |
| | NO: 738) | | |
| 32G1 | RSSQSLVHSNGNTYLH | KVSNRFS (SEQ ID | SQSTHVPLT (SEQ ID NO: 39) |
| | (SEQ ID NO: 14) | NO: 28) | |

TABLE 2B
EU or Kabat light chain HVR consensus sequences

| | HVR Ll |
|---|---|
| Consensus 1 | RXSENXYSXLA (SEQ ID NO: 826) |
| Consensus 2 | RSSQXXXHSNGXTYLX (SEQ ID NO: 827) |
| Consensus 3 | KSSQSXXXSXXQKXXLX (SEQ ID NO: 828) |

TABLE 3A
EU or Kabat heavy chain HVR sequences

| Ab ID | HVR H1 | HVR H2 | HVR H3 |
|---|---|---|---|
| 4D11 | FTLSSYAMS | VASISRGGSTYYP (SEQ ID NO: 66) | TRGYGYYRTPFAN (SEQ ID |
| | (SEQ ID NO: 48) | | NO: 85) |
| 78C5 | FTLSSYAMS | VASISRGGSTYYP (SEQ ID NO: 66) | TRGYGYYRTPFAN (SEQ ID |
| | (SEQ ID NO: 48) | | NO: 85) |
| 6G12 | YTFTEYTMH | IGGINPNNGGTSYS (SEQ ID NO: 67) | ARGGSHYYAMDY (SEQ ID |
| | (SEQ ID NO: 49) | | NO: 86) |
| 8E10 | YTFTDYEMH | IGVIDPETGGTAYN (SEQ ID NO: 68) | TSPDYYGSSYPLYYAMDY (SEQ |
| | (SEQ ID NO: 50) | | ID NO: 87) |
| 7E5 | FTFSDAWMG | VAEIRDKVKNHATYYA (SEQ ID | RLGVFDY (SEQ ID NO: 88) |
| | (SEQ ID NO: 51) | NO: 69) | |
| 7F8 | FSFNTYAMN | IARIRSKSNNYATYYA (SEQ ID | VRHGDGNLWYIDV (SEQ ID |
| | (SEQ ID NO: 52) | NO: 70) | NO: 89) |
| 8F8 | YTVSRYWMH | IGRIDPNSGGTKYN (SEQ ID NO: 71) | VLTGTDFDY (SEQ ID NO: 90) |
| | (SEQ ID NO: 53) | | |
| 1H7 | FSFNTYAMN | IARIRSKSNNYATYYA (SEQ ID | VRHGDGNLWYIDV (SEQ ID |
| | (SEQ ID NO: 52) | NO: 70) | NO: 89) |
| 2H8 | FSFNTYAMN | IARIRSKSNNYATYYA (SEQ ID | VRHGDGNLWYIDV (SEQ ID |
| | (SEQ ID NO: 52) | NO: 70) | NO: 89) |
| 3A2 | YPFSNFWIT | IGDIYPGSDNSNYN (SEQ ID NO: 72) | AREAYYTNPGFAY (SEQ ID |
| | (SEQ ID NO: 54) | | NO: 91) |
| 3A7 | FTFSDAWMG | VAEIRDKVKNHATYYA (SEQ ID | RLGVFDY (SEQ ID NO: 88) |
| | (SEQ ID NO: 51) | NO: 69) | |
| 3B10 | LTSNTYTQT | ESVIRSKSNNFSTLYA (SEQ ID | VRHKSNRYPGVY (SEQ ID |
| | (SEQ ID NO: 55) | NO: 73) | NO: 92) |
| 4F11 | YPFSNFWIT | IGDIYPGSDNSNYN (SEQ ID NO: 72) | AREAYYTNPGFAY (SEQ ID |
| | (SEQ ID NO: 54) | | NO: 91) |
| 6H6 | FTFSDAWMD | VAEIRNKVNNHATYYA (SEQ ID | TSLYDGYYLRFAY (SEQ ID |
| | (SEQ ID NO: 56) | NO: 74) | NO: 93) |
| 7A9 | FTFNTYSMN | VAHIKTKZNNFATFYA (SEQ ID | VZHZSNNYPFAY (SEQ ID |
| | (SEQ ID NO: 57) | NO: 75) | NO: 94) |
| 7B3 | YTFTTYWIH | IGRNDPNSGGSNYN (SEQ ID NO: 76) | VRTNWDGDF (SEQ ID NO: 95) |
| | (SEQ ID NO: 58) | | |
| 8A1 | YAFSNYWMS | IGQIYPGDGDTKYN (SEQ ID NO: 77) | SREKGADYYGSTYSAWFSY |
| | (SEQ ID NO: 59) | | (SEQ ID NO: 96) |
| 9F5 | YAFSSSWMN | IGRIYPGDGDTNYN (SEQ ID NO: 78) | ARLLRNQPGESYAMDY (SEQ ID |
| | (SEQ ID NO: 60) | | NO: 97) |
| 9F5a | YAFSSSWMN | RIYPGDGDTNYNGEFRV (SEQ ID | ARLLRNQPGESYAMDY (SEQ ID |
| | (SEQ ID NO: 60) | NO: 888) | NO: 97) |
| 9G1 | YIFTTYWIH | IGRIDPNNGDTNYN (SEQ ID NO: 79) | VMTGTDFDY (SEQ ID NO: 98) |
| | (SEQ ID NO: 61) | | |
| 9G3 | FNFNTYAMK | IARIRSNSNDYATNYS (SEQ ID | VGHKINNYPFAH (SEQ ID |
| | (SEQ ID NO: 62) | NO: 80) | NO: 99) |
| 10A9 | YPFSNFWIT | IGDIYPGSDNRNFN (SEQ ID NO: 81) | AREAYYTNPGFAY (SEQ ID |
| | (SEQ ID NO: 54) | | NO: 91) |
| 11A8 | FNFNTYAMN | VARIRSKSNNYATYYA (SEQ ID | VRHYSNYGWGFAY (SEQ ID |
| | (SEQ ID NO: 63) | NO: 82) | NO: 100) |
| 12D9 | YTFSDYYIH | IGYIYPNNGDNGYN (SEQ ID NO: 83) | ARRGYYGGSYDY (SEQ ID |
| | (SEQ ID NO: 64) | | NO: 101) |
| 12F9 | FRFNTYAMT | EGVIRRKSSNFATLYA (SEQ ID | VRHKSNKYPFVY (SEQ ID |
| | (SEQ ID NO: 65) | NO: 84) | NO: 102) |
| 10C1 | FTFSDAWMD | VAEIRNKINNHATYYA (SEQ ID | TSLYDGSYLRFAY (SEQ ID |
| | (SEQ ID NO: 56) | NO: 585) | NO: 588) |
| 7E9 | YTFTEYTMH | IGGINPNNGGTSYK (SEQ ID | ARGGSHYYAMDY (SEQ ID |
| | (SEQ ID NO: 49) | NO: 586) | NO: 86) |
| 8C3 | YSFTGYYMH | IGRVNPNNGGTSYN (SEQ ID | VLTGGYFDY (SEQ ID NO: 589) |
| | (SEQ ID NO: 584) | NO: 587) | |
| 1B4 | SRFTFSSYAMS | VAAISGGGRYTYYP (SEQ ID | ARHYDGYLDY (SEQ ID NO: 709) |
| | (SEQ ID NO: 703) | NO: 706) | |
| 6H2 | SAFSLTNYAVH | LGVIWSGGSTAFN (SEQ ID NO: 707) | ATHYYRSTYAFSY (SEQ ID |
| | (SEQ ID NO: 704) | | NO: 710) |
| 7B11v1 | SRFTFSSYAMS | VAAISGGGRYTYYP (SEQ ID | ARHYDGYLDY (SEQ ID NO: 709) |
| | (SEQ ID NO: 703) | NO: 706) | |
| 7B11v2 | SGYTFTDFYMN | IGDINPNNGHTTYN (SEQ ID | AREPYSYGSSPWYFLV (SEQ ID |
| | (SEQ ID NO: 747) | NO: 755) | NO: 763) |
| 18D8 | SRFTFSSYAMS | VAAISGGGRYTYYP (SEQ ID | ARHYDGYLDY (SEQ ID NO: 709) |
| | (SEQ ID NO: 703) | NO: 706) | |
| 18E4v1 | SRFTFSSYAVS | VATISGGGRYTYYP (SEQ ID | ARHYDGYLDY (SEQ ID NO: 709) |
| | (SEQ ID NO: 705) | NO: 708) | |
| 18E4v2 | SGYTFTAYWMH | IGRTHPSDSDTNYN (SEQ ID | ATYSNYVTGAMDS (SEQ ID |
| | (SEQ ID NO: 748) | NO: 756) | NO: 764) |
| 29F6v1 | SRFTFSSYAMS | VAAISGGGRYTYYP (SEQ ID | ARHYDGYLDY (SEQ ID NO: 709) |
| | (SEQ ID NO: 703) | NO: 706) | |
| 29F6v2 | SGFNIKNTYIH | IGRIDPAIGNTNYA (SEQ ID NO: 757) | VSPGMDY (SEQ ID NO: 765) |
| | (SEQ ID NO: 749) | | |
| 40D5v1 | SRFTFSSYAMS | VAAISGGGRYTYYP (SEQ ID | ARHYDGYLDY (SEQ ID NO: 709) |
| | (SEQ ID NO: 703) | NO: 706) | |
| 40D5v2 | SGYTFTNYWIH | IGRIHPSDSDINYN (SEQ ID NO: 758) | VKTGTSFAS (SEQ ID NO: 766) |
| | (SEQ ID NO: 750) | | |
| 43B9 | SRFTFSSYAMS | VAAISGGGRYTYYP (SEQ ID | ARHYDGYLDY (SEQ ID NO: 709) |
| | (SEQ ID NO: 703) | NO: 706) | |
| 44A8 | SRFTFSSYAMS | VAAISGGGRYTYYP (SEQ ID | ARHYDGYLDY (SEQ ID NO: 709) |
| | (SEQ ID NO: 703) | NO: 706) | |
| 44B4v1 | SRFTFSSYAMS | VAAISGGGRYTYYP (SEQ ID | ARHYDGYLDY (SEQ ID NO: 709) |
| | (SEQ ID NO: 703) | NO: 706) | |
| 44B4v2 | SGYTFTSATMH | IGYINPNSGYSKYN (SEQ ID NO: 759) | ARWGIDGNYGGGFEDV (SEQ |
| | (SEQ ID NO: 751) | | ID NO: 767) |
| 45D6 | YSFTDYNIH | IGYINPNSDNTRYI (SEQ ID NO: 760) | TRGFSNLGAMDY (SEQ ID |
| | (SEQ ID NO: 752) | | NO: 768) |
| 29F7 | FTLSNYWMN | VAQIRLKSDNYATHYA (SEQ ID | TGAGGNHENY (SEQ ID NO: 769) |
| | (SEQ ID NO: 753) | NO: 761) | |
| 32G1 | YTFTDYNIH | IGYINPNNGGTTYN (SEQ ID | ATTYVSFSY (SEQ ID NO: 770) |
| | (SEQ ID NO: 754) | NO: 762) | |

TABLE 3B
EU or Kabat heavy chain HVR consensus sequences

| | HVR H1 | HVR H2 |
|---|---|---|
| Consensus 1 | YX₁X₂X₃XYXXH | IGXXXPX₁X₂X₃X₄X₅XYX₆ |
| | X₁ is T or S | X₁ is N or E |
| | X₂ is F or V | X₂ is N, S, or T |
| | X₃ is T ot S | X₃ is G or D |
| | (SEQ ID NO: 829) | X₄ is G, D, or N |
| | | X₅ is T, S, or N |
| | | X₆ is N, S, K, or I |
| | | (SEQ ID NO: 836) |
| Consensus 2 | YTFTXYXXH | IGXXXPNNGGTXYN |
| | (SEQ ID NO: 830) | (SEQ ID NO: 837) |
| Consensus 3 | EITSDAWMX₁ | VAEIRX₁KX₂X₃NHATYYA |
| | X₁ is D or G | X₁ is N or D |
| | (SEQ ID NO: 831) | X₂ is V or I |
| | | X₃ is N or K |
| | | (SEQ ID NO: 838) |
| Consensus 4 | FXX₁X₂X₃YX₄MX₅ | XX₁XIX₂X₃X₄X₅X₆X₇X₈ATXYX₉ |
| | X₁ is F or L | X₁ is A or G |
| | X₂ is N or S | X₂ is R or K |
| | X₃ is T or N | X₃ is S, T, R, or L |
| | X₄ is A, S, or W | X₄ is K or N |
| | X₅ is N, K, or T | X₅ is S, E, or Q |
| | (SEQ ID NO: 832) | X₆ is N, S, or D |
| | | X₇ is N or D |
| | | X₈ is Y or F |
| | | X₉ is A or S |
| | | (SEQ ID NO: 839) |
| Consensus 5 | FXFNTYAMN | XAXIRSKSNNYATXYA |
| | (SEQ ID NO: 833) | (SEQ ID NO: 840) |
| Consensus 6 | YXFX₁X₂XVVX₃X | IGXIX₁PX₂XX₃X₄X₅X₆X₇N |
| | X₁ is S or T | X₁ is Y or D |
| | X₂ is N, S, or T | X₂ is G or N |
| | X₃ is I or M | X₃ is G or D |
| | (SEQ ID NO: 834) | X₄ is N or D |
| | | X₅ is T, R, or S |
| | | X₆ is N or K |
| | | X₇ is Y or F |
| | | (SEQ ID NO: 841) |
| Consensus 7 | YXFSNXWIX | IGXIYPGXGDTNYN |
| | (SEQ ID NO: 835) | (SEQ ID NO: 842) |

TABLE 4A
EU or Kabat light chain Framework sequences

| Ab ID | VL FR1 | VL FR2 | VL FR3 | VL FR4 |
|---|---|---|---|---|
| 4D11 | DIZVTQSPASLSA | WYQLKQGKSPQLLV | GVPSRFSGSGSGTQFS | FGGGTKLEIK (SEQ |
| | SVGETVTITC | Y (SEQ ID NO: 120) | LRINSLQPEDEGSYYC | ID NO: 148) |
| | (SEQ ID NO: 103) | | (SEQ ID NO: 130) | |
| 78C5 | DIZVTQSPASLSA | WYQLKQGKSPQLLV | GVPSRFSGSGSGTQFS | FGGGTKLEIK (SEQ |
| | SVGETVTITC | Y (SEQ ID NO: 120) | LRINSLQPEDEGSYYC | ID NO: 148) |
| | (SEQ ID NO: 103) | | (SEQ ID NO: 130) | |
| 6G12 | TMSQSPSSLAVS | WYQQKPGQSPKLLIY | GVPDRFTGSGSGTDFT | FGAGTKLELK |
| | VGEKVTMSC | (SEQ ID NO: 121) | LTISSVKAEDLAVYYC | (SEQ ID NO: 149) |
| | (SEQ ID NO: 104) | | (SEQ ID NO: 131) | |
| 8F11 | DVZMTQTPLTLS | WLLQRPGQSPKRLIY | GVPDRFAGSGSGTDFT | FGAGTKLELK |
| | VTIGQPASISC | (SEQ ID NO: 122) | LKISRLEADDLGIYYC | (SEQ ID NO: 149) |
| | (SEQ ID NO: 105) | | (SEQ ID NO: 132) | |
| 8E10 | DVZMTQTPLTLS | WLLQRPGQSPKRLIY | GVPDRFTGSGSGTDFT | FGGGTKLEIK (SEQ |
| | VTIGQPASISC | (SEQ ID NO: 122) | LKISRVEAEDLGVYYC | ID NO: 148) |
| | (SEQ ID NO: 105) | | (SEQ ID NO: 133) | |
| 7E5 | DVZMTQTPLTLS | WLLQRPGQSPKRLIY | GVPDRFAGSGSGTDFT | FGAGTKLELK |
| | VTIGQPASISC | (SEQ ID NO: 122) | LKISRLEADDLGIYYC | (SEQ ID NO: 149) |
| | (SEQ ID NO: 105) | | (SEQ ID NO: 132) | |
| 7E5v2 | DVVMTQTPLTLS | WLLQRPGQSPKRLIY | GVPDRFAGSGSGTDFT | FGAGTKLELK |
| | VTIGQPASISC | (SEQ ID NO: 122) | LKISRLEADDLGIYYC | (SEQ ID NO: 149) |
| | (SEQ ID NO: 111) | | (SEQ ID NO: 132) | |
| 7F8 | VLTQSPALMSAS | WYQQKPRSSPKPWIY | GVPARFSGSGSGTSYS | FGGGTKLVIK |
| | PGEKVTMTC | (SEQ ID NO: 123) | LTINNMEAEDAATYY | (SEQ ID NO: 150) |
| | (SEQ ID NO: 106) | | C (SEQ ID NO: 134) | |
| 8F8 | DVZMTQTPLSLP | WYLQKPGQSPKLLIY | GVPDRFSGSGSGTDFT | FGAGTKLELK |
| | VSLGDQASISC | (SEQ ID NO: 124) | LKISRVEAEDLGVYFC | (SEQ ID NO: 149) |
| | (SEQ ID NO: 107) | | (SEQ ID NO: 135) | |
| 1H7 | VLTQSPAIMZASP | WYQQKPRSSPKPWIY | GVPARFSGSGSGTSYS | FGGGTKLVIK |
| | GEKVTMTC (SEQ | (SEQ ID NO: 123) | LTISSMEAEDAATYYC | (SEQ ID NO: 150) |
| | ID NO: 108) | | (SEQ ID NO: 136) | |
| 2H8 | NVLTQSPALMSA | WYQQKPRSSPKPWIY | GVPARFSGSGSGTSYS | FGGGTKLVIK |
| | SPGEKVTMTC | (SEQ ID NO: 123) | LTISSMEAEDAATYYC | (SEQ ID NO: 150) |
| | (SEQ ID NO: 109) | | (SEQ ID NO: 136) | |
| 3A2 | DVVMTQTPLSLP | WYLRKPGQSPKLLIY | GVPDRFSGSGSGTDFT | FGGGTELEIK (SEQ |
| | VSLGDQASISC | (SEQ ID NO: 125) | LKISRVEAEDLGVYYC | ID NO: 151) |
| | (SEQ ID NO: 110) | | (SEQ ID NO: 137) | |
| 3A7 | DVVMTQTPLTLS | WLLQRPGQSPKRLIY | GVPDRFAGSGSGTDFT | FGGGTKLEMK |
| | VTIGQPASISC | (SEQ ID NO: 122) | ZKISRLEADDLGIYYC | (SEQ ID NO: 152) |
| | (SEQ ID NO: 111) | | (SEQ ID NO: 138) | |
| 3B10 | ITMSQSPSSLAVS | WYQQKPGQSPKLLIY | GVPDRFTGSGSGTDFT | FGAGTKLELK |
| | VGEKVTMSC | (SEQ ID NO: 121) | LTISSVKAEDLAVYCC | (SEQ ID NO: 149) |
| | (SEQ ID NO: 112) | | (SEQ ID NO: 139) | |
| 4F11 | DVZMTQTPLSLP | WYLRKPGQSPKLLIY | GVPDRFSGSGSGTDFT | FGGGTELEIK (SEQ |
| | VSLGDQASISC | (SEQ ID NO: 125) | LKISRVEGEDLGVYYC | ID NO: 151) |
| | (SEQ ID NO: 107) | | (SEQ ID NO: 140) | |
| 6H6 | QTQSPSSLAVSA | WYQQKPGQSPKLLIS | GVPDRFTGSGFGTDFT | FGAGTKLELK |
| | GEKVTLSC (SEQ | (SEQ ID NO: 593) | LTISSVQGEDLAVYYC | (SEQ ID NO: 149) |
| | ID NO: 590) | | (SEQ ID NO: 594) | |
| 7A9 | QMSQSPACLZAZ | WYQQKQGKSPKLVV | GVPSRFSGRGSGTQFF | FGSGTKLEIK (SEQ |
| | VGESVTITC (SEQ | Y (SEQ ID NO: 126) | LKINSZQREDFGSYYC | ID NO: 153) |
| | ID NO: 113) | | (SEQ ID NO: 141) | |
| 8A1 | DIQMTQSPASLSV | WYQQKQGKSPQLLV | GVPSRFSASGSATQFS | FGGGTKLEMN |
| | SVGETVTITC | Y (SEQ ID NO: 127) | LKINSLQSADFGSYYC | (SEQ ID NO: 154) |
| | (SEQ ID NO: 114) | | (SEQ ID NO: 142) | |
| 9F5 | DVZMTQNPLSLP | WYLQKPGQSPKLLIY | GVPDRFSGSGSGTDFT | FGGGTKLEIK (SEQ |
| | VSLGDQASISC | (SEQ ID NO: 124) | LKISRVEADDLGVYLC | ID NO: 148) |
| | (SEQ ID NO: 115) | | (SEQ ID NO: 143) | |
| 9F5v2 | DVVMTQTPLSLP | WYLQKPGQSPKLLIY | GVPDRFSGSGSGTDFT | FGGGTKLEIK (SEQ |
| | VSLGDQASISC | (SEQ ID NO: 124) | LKISRVEADDLGVYFC | ID NO: 148) |
| | (SEQ ID NO: 110) | | (SEQ ID NO: 848) | |
| 9G1 | DVLMTQTPLSLP | WYLQKPGQSPKLLIY | GVPDRFSGSGSGTDFT | FGGGTKLEIK (SEQ |
| | VSLGDQASISC | (SEQ ID NO: 124) | LRISGVEAEDLGVYFC | ID NO: 148) |
| | (SEQ ID NO: 116) | | (SEQ ID NO: 144) | |
| 9G3 | NVLTQSPALIWA | WXXXKPRSSPKPGIY | GVPGRFSGSGSGTYXS | FGGGTKLEMK |
| | ZPGEKVTMTC | (SEQ ID NO: 128) | FKISSMEGKMGPLIIFC | (SEQ ID NO: 155) |
| | (SEQ ID NO: 117) | | (SEQ ID NO: 145) | |
| 10A9 | DVVMTQTPLSLP | WYLRKPGQSPKLLIY | GVPDRFSGSGSGTDFT | FGGGTELEIK (SEQ |
| | VSLGDQASISC | (SEQ ID NO: 125) | LKISRVEAEDLGVYYC | ID NO: 151) |
| | (SEQ ID NO: 110) | | (SEQ ID NO: 137) | |
| 11A8 | DIZMTQSPSSLTV | WYQQKPGQPZKLLIY | GVRDRFTGSGZGTDFT | FGGGTKLEMK |
| | TAGEKVTMSC | (SEQ ID NO: 129) | LTISSVQGEDLAIYYC | (SEQ ID NO: 152) |
| | (SEQ ID NO: 118) | | (SEQ ID NO: 146) | |
| 12D9 | TQSPSSLAVSVGE | WYQQKPGQSPKLLIY | GVPDRFTGSGSGTDFT | FGSGTKLEIK (SEQ |
| | KVTMTC (SEQ ID | (SEQ ID NO: 121) | LTISTVKAEDLAVYYC | ID NO: 153) |
| | NO: 119) | | (SEQ ID NO: 147) | |
| 12F9 | TMSQSPSSLAVS | WYQQKPGQSPKLLIY | GVPDRFTGSGSGTDFT | FGAGTKLELK |
| | VGEKVTMSC | (SEQ ID NO: 121) | LTISSVKAEDLAVYCC | (SEQ ID NO: 149) |
| | (SEQ ID NO: 104) | | (SEQ ID NO: 139) | |
| 10C1 | QTQVFLSLLLWV | WYQQKPGQSPKLLIS | GVPDRFTGSGSGTDFT | FGAGTKLELK |
| | SGTCGNIMLTQSP | (SEQ ID NO: 593) | LTINSVQAEDLAVYYC | (SEQ ID NO: 149) |
| | SSLAVSAGEKVT | | (SEQ ID NO: 595) | |
| | LSC (SEQ ID | | | |
| | NO: 591) | | | |
| 7E9 | DIVMSQSPSSLAV | WYQQKPGQSPKLLIY | GVPDRFTGSGSGTDFT | FGAGTKLELK |
| | SVGEKVTMSC | (SEQ ID NO: 121) | LTISSVKAEDLAVYYC | (SEQ ID NO: 149) |
| | (SEQ ID NO: 592) | | (SEQ ID NO: 131) | |
| 8C3 | DVVMTQTPLSLP | WYLQKPGQSPKLLIY | GVPDRFSGSGSGTDFT | FGSGTKLEIK (SEQ |
| | VSLGDQASISC | (SEQ ID NO: 124) | LKISRVEAEDLGVYFC | ID NO: 153) |
| | (SEQ ID NO: 110) | | (SEQ ID NO: 135) | |
| 1B4v1 | DIVMTQSHKFMS | WYQQKPGQSPKLLIY | GVPDRFTGSGFGTDFT | FGGGTKLEIK (SEQ |
| | TSVGDRVSITCK | (SEQ ID NO: 121) | FTISSVQAEDLAVYYC | ID NO: 148) |
| | A (SEQ ID | | (SEQ ID NO: 715) | |
| | NO: 711) | | | |
| 1B4v2 | ZVVZTQTPLSLPV | WFLQKPGQSPKLLIF | GVPDRFSGSGSGTDFT | FGAGTKLELK |
| | SLGDQASFSCRS | (SEQ ID NO: 777) | LKISRVEAEDLGVYFC | (SEQ ID NO: 149) |
| | (SEQ ID NO: 771) | | (SEQ ID NO: 135) | |
| 6H2 | DVLMTQTPLSLP | WYLQKPGQSPKLLIY | GVPDRFSGSGSGTDFT | FGSGTKLEIK (SEQ |
| | VSLGDQASISCRS | (SEQ ID NO: 124) | LKISRVEAEDLGVYYC | ID NO: 153) |
| | (SEQ ID NO: 712) | | (SEQ ID NO: 137) | |
| 7B11 | DIVMTQSHKFMS | WYQQKPGQSPKLLIY | GVPDRFTGSGSGTDYT | FGGGTKLEIK (SEQ |
| | TSVGDRVSITCK | (SEQ ID NO: 121) | LTISSVQAEDLALYYC | ID NO: 148) |
| | A (SEQ ID | | (SEQ ID NO: 716) | |
| | NO: 711) | | | |
| 18D8 | DIVMTQSHKFMS | WYQQKPGQSPKLLIY | GVPDRFTGSGFGTDFT | FGGGTKLEIK (SEQ |
| | TSIGARVSITCKA | (SEQ ID NO: 121) | FTISSVQAEDLAVYYC | ID NO: 148) |
| | (SEQ ID NO: 713) | | (SEQ ID NO: 715) | |
| 18E4v1 | DIVMTQSPKSMS | WYQQKPEQSPKLLIY | GVPDRFTGSGSATDFT | FGGGTKLEIK (SEQ |
| | MSVGERVTLTCK | (SEQ ID NO: 714) | LTISSVQAEDLADYHC | ID NO: 148) |
| | A (SEQ ID | | (SEQ ID NO: 717) | |
| | NO: 714) | | | |
| 18E4v2 | NIVMTQSPKSMS | WYQQKPEQSPKLLIY | GVPDRFTGSGSATDFT | FGGGTKLEIK (SEQ |
| | MSVGERVTLTCK | (SEQ ID NO: 714) | LTISSVQAEDLADYHC | ID NO: 148) |
| | A (SEQ ID | | (SEQ ID NO: 717) | |
| | NO: 772) | | | |
| 18E4v3 | DVVMTQTPLSLP | WYLQKPGQSPKLLIY | GVPDRFSGSGSGTDFT | FGAGTKLELK |
| | VSLGDQASISCRS | (SEQ ID NO: 124) | LRISRVEAEDLGVYFC | (SEQ ID NO: 149) |
| | (SEQ ID NO: 773) | | (SEQ ID NO: 781) | |
| 29F6v1 | DIVMTQSHKFMS | WYQQKPGQSPKLLIY | GVPDRFTGSGSGTDFT | FGGGTKLEIK (SEQ |
| | TSIGARVSITCKA | (SEQ ID NO: 121) | FTISSVQAEDLAVYYC | ID NO: 148) |
| | (SEQ ID NO: 713) | | (SEQ ID NO: 718) | |
| 29F6v2 | DVVMTQTPLSLP | WYLQKPGQSPKLLIY | GVPDRFSGSGSGTDFT | FGAGTKLELK |
| | VSLGDQASISCRS | (SEQ ID NO: 124) | LKISRVEAEDLGVYFC | (SEQ ID NO: 149) |
| | (SEQ ID NO: 773) | | (SEQ ID NO: 135) | |
| 40D5 | DIVMTQSHKFMS | WYQQKPGQSPKLLIY | GVPDRFTGSGSGTDFT | FGGGTKLEIK (SEQ |
| | TSIGARVSITCKA | (SEQ ID NO: 121) | FTISSVQAEDLAVYYC | ID NO: 148) |
| | (SEQ ID NO: 713) | | (SEQ ID NO: 718) | |
| 43B9 | DIVMTQSHKFMS | WYQQKPGQSPKLLIY | GVPDRFTGSGSGTDFT | FGGGTKLEIK (SEQ |
| | TSIGARVSITCKA | (SEQ ID NO: 121) | FTISSVQAEDLAVYYC | ID NO: 148) |
| | (SEQ ID NO: 713) | | (SEQ ID NO: 718) | |
| 44A8v1 | IDIVMTQSHKFMS | WYQQKPGQSPKLLIY | GVPDRFTGSGSGTDFT | FGGGTKLEIK (SEQ |
| | TSVGDRVSITCK | (SEQ ID NO: 121) | FTISSVQAEDLAVYYC | ID NO: 148) |
| | A (SEQ ID | | (SEQ ID NO: 718) | |
| | NO: 711) | | | |
| 4418v2 | DIVLTQSPASLAV | WYQQKPGQPPKLLIY | GVPARFSGSGSGTDFS | FGGGTKLEIK (SEQ |
| | SLGQRATISCRA | (SEQ ID NO: 778) | LNIHPVEEDDIAMYFC | ID NO: 148) |
| | (SEQ ID NO: 774) | | (SEQ ID NO: 782) | |
| 44B4v1 | DIVMTQSHKFMS | WYQQKPGQSPKLLIY | GVPDRFTGSGSGTDFT | FGGGTKLEIK (SEQ |
| | TSIGARVSITCKA | (SEQ ID NO: 121) | FTISSVQAEDLAVYYC | ID NO: 148) |
| | (SEQ ID NO: 713) | | (SEQ ID NO: 718) | |
| 44B4v2 | DIQMTQFPASLA | WYQQKQGKSPQLLIY | GVPSRFSGSGSGTQYS | FGGGTKLEIK (SEQ |
| | AZVGESVTITCRA | (SEQ ID NO: 779) | MKINSMQPEDTAIYFC | ID NO: 148) |
| | (SEQ ID NO: 775) | | (SEQ ID NO: 783) | |
| 29F7 | ILLTQSPAILSVSP | WYQQRTNGSPRLLIK | GIPSRFSGSGSGTDFTL | FGAGTKLELK |
| | GERVSFSC | (SEQ ID NO: 780) | NINSVESEDIADYYC | (SEQ ID NO: 149) |
| | (SEQ ID NO: 776) | | (SEQ ID NO: 784) | |
| 32G1 | DVVMTQTPLSLP | WYLQKPGQSPKLLIY | GVPDRFSGSGSGTDFT | FGAGTKLELK |
| | VSLGDQASISC | (SEQ ID NO: 124) | LKISRVEAEDLGVYFC | (SEQ ID NO: 149) |
| | (SEQ ID NO: 110) | | (SEQ ID NO: 135) | |

TABLE 4B
EU or Kabat heavy chain Framework sequences

| Ab ID | VH FR1 | VH FR2 | VH FR3 | VH FR4 |
|---|---|---|---|---|
| 4D11 | EVKLVESGGGLVK | WVRQTPEKRLEW | DSVQGRFTFSRDNARN | WGQGTLVTVSA |
| | PGGSLKLSCAASG | (SEQ ID NO: 175) | ILYLQMSSLRSEDTAM | (SEQ ID NO: 209) |
| | (SEQ ID NO: 156) | | YYC (SEQ ID NO: 188) | |
| 78C5 | EVKLVESGGGLVK | WVRQTPEKRLEW | DSVQGRFTFSRDNARN | WGQGTLVTVSA |
| | PGGSLKLSCAASG | (SEQ ID NO: 175) | ILYLQMSSLRSEDTAM | (SEQ ID NO: 209) |
| | (SEQ ID NO: 156) | | YYC (SEQ ID NO: 188) | |
| 6G12 | EVQLQQSGPELVKP | WVKQSHGKSLEW | QKFKGKASLTVDKSSS | WGQGTSVTVSS |
| | GTSVKISCKTSG | (SEQ ID NO: 176) | TAYMELHSLASDDSA | (SEQ ID NO: 210) |
| | (SEQ ID NO: 157) | | VYYC (SEQ ID NO: 189) | |
| 8E10 | QVQLQQSGAELVR | WVKQTPVHGLEW | QKFKGKAILTADKSSS | WGQGTSVTVSS |
| | PGASVTLSCKASG | (SEQ ID NO: 177) | TAYMELRSLTSEDSAV | (SEQ ID NO: 210) |
| | (SEQ ID NO: 158) | | YYC (SEQ ID NO: 190) | |
| 7E5 | EVKLEESGGGLVQ | WVRQSPEKGLEW | ESVKGRFTISRDDSKST | WGQGTTLTVSS |
| | PGGSMKLSCAASG | (SEQ ID NO: 178) | VYLQMNTLRADDTGI | (SEQ ID NO: 211) |
| | (SEQ ID NO: 159) | | YYC (SEQ ID NO: 191) | |
| 7F8 | EVQLVESGGGLVQ | WVRQAPGKGLEW | DSVKDRITCSRDDSEN | WGTGTTVTVST |
| | PKGSLKLSCAASG | (SEQ ID NO: 179) | MFYLQLSSLKTEDTA | (SEQ ID NO: 212) |
| | (SEQ ID NO: 160) | | MYYC (SEQ ID NO: 192) | |
| 8F8 | QVQLQQSGAELVK | WVKQRPGRGLEW | EKFKTKATLTVDKPSS | WGQGTTLTVSS |
| | PGASVKLSCKASG | (SEQ ID NO: 180) | TAYMQVSSLTSEDSAV | (SEQ ID NO: 211) |
| | (SEQ ID NO: 161) | | YYC (SEQ ID NO: 193) | |
| 1H7 | ZVQLVESGGGLVQ | WVRQAPGKGLEW | DSVKDRFTCSRDDSEN | WGTGTTVTVSS |
| | PKGSLKLSCAASG | (SEQ ID NO: 179) | MFYLQLSSLKTEDTAI | (SEQ ID NO: 213) |
| | (SEQ ID NO: 162) | | YYC (SEQ ID NO: 194) | |
| 2H8 | EVQLVESGGGLVQ | WVRQAPGKGLEW | DSVKDRFTCSRDDSEN | WGTGTTVTVSS |
| | PKGSLKLSCAASG | (SEQ ID NO: 179) | MFYLQLSSLKTEDTA | (SEQ ID NO: 213) |
| | (SEQ ID NO: 160) | | MYYC (SEQ ID NO: 195) | |
| 3A2 | QVQLQQSGAELVK | WVKQRPGQGLVW | EKFKTKATLTVDTSSS | WGQGTLVTVST |
| | PGASVKMSCKTSG | (SEQ ID NO: 181) | TAYMHLSSLTSEDSAV | (SEQ ID NO: 214) |
| | (SEQ ID NO: 163) | | YFC (SEQ ID NO: 196) | |
| 3A7 | EVKLEESGGGLVQ | WVRQSPEKGLEW | ESVKGRFTISRDDSKST | WGQGTTLTVSS |
| | PGGSMKLSCAASG | (SEQ ID NO: 178) | VYLQMNTLRADDTGI | (SEQ ID NO: 211) |
| | (SEQ ID NO: 159) | | YYC (SEQ ID NO: 191) | |
| 3B10 | EVQLVZZGRGZSQ | GVPQGPGKGREW | DSVKDRFTZSRDDSES | WGQGTIVTVS |
| | GKGSXZZGRAZRC | (SEQ ID NO: 182) | LFYZQMSZZKZEDTA | (SEQ ID NO: 215) |
| | (SEQ ID NO: 164) | | MYYZ (SEQ ID NO: 197) | |
| 4F11 | QVQLQQSGAELVK | WVKQRPGQGLVW | EKFKTKATLTVDTSSS | WGQGTLVTVST |
| | PGASVKMSCKTSG | (SEQ ID NO: 181) | TAYMHLSSLTSEDSAV | (SEQ ID NO: 214) |
| | (SEQ ID NO: 163) | | YFC (SEQ ID NO: 196) | |
| 6H6 | EVKLEESGGGLVQ | WVRQSPEKGLEW | ESVKGRFTISRDDSKST | WGQGTLVTVSA |
| | PGGSMKLSCTASG | (SEQ ID NO: 178) | VYLQMNSLRTEDTGIY | (SEQ ID NO: 209) |
| | (SEQ ID NO: 165) | | YC (SEQ ID NO: 198) | |
| 7A9 | LSCAASG (SEQ ID | WVRQAPGKGLEW | DSVKDRFTISRDDSES | WGQGTLVTVSA |
| | NO: 166) | (SEQ ID NO: 179) | MLYLQMZNLKTEDTA | (SEQ ID NO: 209) |
| | | | MYYC (SEQ ID NO: 199) | |
| 7B3 | QVQLQQSGAVLVK | WVKQRPGRGPEW | EKFRNKAILTVDKPSS | WGQGTTLTVSS |
| | PGASVKLSCKASG | (SEQ ID NO: 183) | TAYMQLNSLTSEDZA | (SEQ ID NO: 211) |
| | (SEQ ID NO: 167) | | VYYC (SEQ ID NO: 200) | |
| 8A1 | EVQLQQSGAELVK | WVKQRPGKGLEW | GKFEGKATLTADKSSS | WGQGTLVTVSA |
| | PGASVKISCKASG | (SEQ ID NO: 184) | TAYMQLSSLTSEDSAV | (SEQ ID NO: 209) |
| | (SEQ ID NO: 168) | | YFC (SEQ ID NO: 201) | |
| 9F5 | QVQLQQSGPELVK | WVKQRPGKGLEW | GEFRVRATLTADTSST | WGQGASVTVSS |
| | PGASLKISCKASG | (SEQ ID NO: 184) | TAYMQLSSLTSEDSAV | (SEQ ID NO: 216) |
| | (SEQ ID NO: 169) | | YFC (SEQ ID NO: 202) | |
| 9G1 | QVQLQQSGAELVK | WVKQRPGRGPEW | EKFKTKATLTVDKPSS | WGQGTTLTVSS |
| | PGASVKLSCKASG | (SEQ ID NO: 183) | TADMQLSSLTSEDSAV | (SEQ ID NO: 211) |
| | (SEQ ID NO: 161) | | YYC (SEQ ID NO: 203) | |
| 9G3 | EVQLVESGGGLVQ | WVRQTPGKGLEW | DSVKDRFTISRDDSESI | WGRGTLV (SEQ ID |
| | PKGSLKLSCAAFG | (SEQ ID NO: 185) | VYVQMNNLKTEDTG | NO: 217) |
| | (SEQ ID NO: 170) | | MYSC (SEQ ID NO: 204) | |
| 10A9 | QVQLQQSGAEVVK | WVKQRPGQGLVW | ERFKTKATLTVDTSSS | WGQGTLVTVSA |
| | PGASVKMSCKTSG | (SEQ ID NO: 181) | TAYMHLSSLTSEDSAV | (SEQ ID NO: 209) |
| | (SEQ ID NO: 171) | | YFC (SEQ ID NO: 205) | |
| 11A8 | EVQLVESGGRLVQ | WVRQAPGKGLEW | DSVKDRFTISRDDSES | WGQGTLVTVSA |
| | PKGSLKLSCAASG | (SEQ ID NO: 179) | MLYLQMNNLKTEDTA | (SEQ ID NO: 209) |
| | (SEQ ID NO: 172) | | MYYC (SEQ ID NO: 206) | |
| 12D9 | QVQLQQYGPELVK | WMKQSHGKSLEW | QEFKGKATLTVDKSSS | WGQGT (SEQ ID |
| | PGASVKMSCKVSG | (SEQ ID NO: 186) | TAYMELRSLTFEDSAV | NO: 218) |
| | (SEQ ID NO: 173) | | YZC (SEQ ID NO: 207) | |
| 12F9 | WRIGQGKGSLKLA | RVRQGPGKGREW | DSVKDRFRASRDDSES | WGQGTLVTVSA |
| | RAARG (SEQ ID | (SEQ ID NO: 187) | MLYVQMSNWKQEDT | (SEQ ID NO: 209) |
| | NO: 174) | | AMYYG (SEQ ID | |
| | | | NO: 208) | |
| 10C1 | GVQSEVKFEESGG | WVRQSPEKGLEW | ESVKGRFTISRDDSKSS | WGQGTLVTVSA |
| | GLVQPGGSMKLSC | (SEQ ID NO: 178) | VSLQMNSLRTEDTGIY | (SEQ ID NO: 209) |
| | TASG (SEQ ID | | YC (SEQ ID NO: 599) | |
| | NO: 596) | | | |
| 7E9 | QVQLQQSGPELVK | WVKQSHGKSLEW | QKFKGKATLTVDRSSS | WGQGTSVTVSS |
| | PGASVKISCKTSG | (SEQ ID NO: 176) | TAYMELRSLTSEDSAV | (SEQ ID NO: 210) |
| | (SEQ ID NO: 597) | | YYC (SEQ ID NO: 600) | |
| 8C3 | QVQLQQSGPDLVK | WVKQSHGKSLEW | QKFKGKAILTVDKSSS | WGQGTTLTVSS |
| | PGASVKISCKASG | (SEQ ID NO: 176) | TAYMELRSLTSEDSAV | (SEQ ID NO: 211) |
| | (SEQ ID NO: 598) | | YYC (SEQ ID NO: 601) | |
| 1B4 | EVQLVESGGGLVK | WVRQTPEKRLEW | DSMKGRFTISRDNAKN | WGQGTTLTVSS |
| | PGGSLKLSCEA | (SEQ ID NO: 175) | FLYLQMSSLRSEDTAM | (SEQ ID NO: 211) |
| | (SEQ ID NO: 719) | | YYC (SEQ ID NO: 722) | |
| 6H2 | QVQLQESGPGLVQ | WIRQSPGKGLEW | AAFISRLNISKDNSKSQ | WGQGTLVTVSA |
| | PSQSLSIICTV (SEQ | (SEQ ID NO: 721) | VFFKMNSLQSDDTAIY | (SEQ ID NO: 209) |
| | ID NO: 720) | | YC (SEQ ID NO: 723) | |
| 7B11v1 | EVQLVESGGGLVK | WVRQTPEKRLEW | DSMKGRFTISRDNAKN | WGQGTTLTVSS |
| | PGGSLKLSCEA | (SEQ ID NO: 175) | FLYLQMSSLRSEDTAM | (SEQ ID NO: 211) |
| | (SEQ ID NO: 719) | | YYC (SEQ ID NO: 722) | |
| 7B11v2 | EVQZQQSGPELVKP | WVKQSLGKSLEW | QKFKGKATLTVDKSSS | RGTGTTVTV (SEQ |
| | GASVKISCKA (SEQ | (SEQ ID NO: 793) | TAYMELRSLTZEESAV | ID NO: 806) |
| | ID NO: 785) | | YYC (SEQ ID NO: 798) | |
| 18D8 | EVQLVESGGGLVK | WVRQTPEKRLEW | DSMKGRFTISRDNAKN | WGQGTTLTVSS |
| | PGGSLKLSCEA | (SEQ ID NO: 175) | FLYLQMSSLRSEDTAM | (SEQ ID NO: 211) |
| | (SEQ ID NO: 719) | | YYC (SEQ ID NO: 722) | |
| 18E4v1 | EVQLVESGGGLVK | WVRQTPEKRLEW | DSMKGRFTISRDNAKN | WGQGTTLTVSS |
| | PGGSLKLSCEA | (SEQ ID NO: 175) | FLYLQMSSLRSEDTAM | (SEQ ID NO: 211) |
| | (SEQ ID NO: 719) | | YYC (SEQ ID NO: 722) | |
| 18E4v2 | QVQLQQPGAELVK | WVKEKPGQGLEW | HNFKGKATLTVDKSSS | WGQGTSVTVSS |
| | PGASVKVSCKA | (SEQ ID NO: 794) | TAYMQLNSLTSEDSA | (SEQ ID NO: 210) |
| | (SEQ ID NO: 786) | | VYYC (SEQ ID NO: 799) | |
| 29F6v1 | EVQLVESGGGLVK | WVRQTPEKRLEW | DSMKGRFTISRDNAKN | WGQGTTLTVSS |
| | PGGSLKLSCEA | (SEQ ID NO: 175) | FLYLQMSSLRSEDTAM | (SEQ ID NO: 211) |
| | (SEQ ID NO: 719) | | YYC (SEQ ID NO: 722) | |
| 29F6v2 | QVQLQQSVAELVR | WVKQRPEQGLEW | PKFQATATITVATSSNS | WGHGTSVTVSS |
| | PGASVKLSCTA | (SEQ ID NO: 795) | AYLQLSSLASEDTAIY | (SEQ ID NO: 807) |
| | (SEQ ID NO: 787) | | YC (SEQ ID NO: 800) | |
| 40D5v1 | EVQLVESGGGLVK | WVRQTPEKRLEW | DSMKGRFTISRDNAKN | WGQGTTLTVSS |
| | PGGSLKLSCEA | (SEQ ID NO: 175) | FLYLQMSSLRSEDTAM | (SEQ ID NO: 211) |
| | (SEQ ID NO: 719) | | YYC (SEQ ID NO: 722) | |
| 40D5v2 | QVQLQQSGAELVK | WVKQRPGQGLEW | QKFKGKATLTVDKSSS | WSQGTLVTVS |
| | PGASVKVSCKA | (SEQ ID NO: 796) | TAYMQILSSLTSEDSA | (SEQ ID NO: 808) |
| | (SEQ ID NO: 788) | | VYYC (SEQ ID NO: 801) | |
| 43B9 | EVQLVESGGGLVK | WVRQTPEKRLEW | DSMKGRFTISRDNAKN | WGQGTTLTVSS |
| | PGGSLKLSCEA | (SEQ ID NO: 175) | FLYLQMSSLRSEDTAM | (SEQ ID NO: 211) |
| | (SEQ ID NO: 719) | | YYC (SEQ ID NO: 722) | |
| 44A8 | EVQLVESGGGLVK | WVRQTPEKRLEW | DSMKGRFTISRDNAKN | WGQGTTLTVSS |
| | PGGSLKLSCEA | (SEQ ID NO: 175) | FLYLQMSSLRSEDTAM | (SEQ ID NO: 211) |
| | (SEQ ID NO: 719) | | YYC (SEQ ID NO: 722) | |
| 44B4v1 | EVQLVESGGGLVK | WVRQTPEKRLEW | DSMKGRFTISRDNAKN | WGQGTTLTVSS |
| | PGGSLKLSCEA | (SEQ ID NO: 175) | FLYLQMSSLRSEDTAM | (SEQ ID NO: 211) |
| | (SEQ ID NO: 719) | | YYC (SEQ ID NO: 722) | |
| 44B4v2 | XXXXXQSGTELAR | WVKQRPGQGLEW | QKFKDKATLTADKSSS | WGTGTTVTVSS |
| | PGASVKMPCKA | (SEQ ID NO: 796) | TAYMQLSSLTSEESAV | (SEQ ID NO: 213) |
| | (SEQ ID NO: 789) | | YYC (SEQ ID NO: 802) | |
| 45D6 | QVQLQQSGRELVK | WVIQSHGESLEW | QKFKGKATLTVNKSSS | WGQGTSVTVSS |
| | PGASVKMSCMSSG | (SEQ ID NO: 797) | TAYMELRSLTSEDSAV | (SEQ ID NO: 210) |
| | (SEQ ID NO: 790) | | YYC (SEQ ID NO: 803) | |
| 29F7 | QVKLEESGGGLVQ | WVRQSPEKGLEW | ESVKGRFTISRDDSKSS | WGQGTTLTVSS |
| | PGGSMKLSCVASG | (SEQ ID NO: 178) | VYLQMNNLRAVDTGI | (SEQ ID NO: 211) |
| | (SEQ ID NO: 791) | | YYC (SEQ ID NO: 804) | |
| 32G1 | QVQLQQSGPELVK | WVKQSHGKSLEW | QKFKGKATLTVNKSSS | WGQGTLVTVSA |
| | PGASVQMSCEASG | (SEQ ID NO: 176) | TAYIELRSLTSEDSAV | (SEQ ID NO: 209) |
| | (SEQ ID NO: 792) | | YHC (SEQ ID NO: 805) | |

Characterization of TREM2 Antibody Binding

Initial characterization of TREM2 antibodies involved determining their ability to bind TREM2 expressed on macrophages and other primary human or mouse immune cells. Cells were harvested, plated at 10⁵/ml in a 96 well plate, washed, and incubated in 100 ul PBS containing 10-50 ug/ml Mab and Fc blocking reagent for 1 hour in ice. Cells were then washed twice and incubated in 100 ul PBS containing 5 ug/ml PE-conjugated secondary antibody for 30 minutes in ice. Cells were washed twice in cold PBS and acquired on a BD FACS Canto. Data analysis and calculation of mean fluorescence intensity (MFI) values or % positive cells was performed with FlowJo (TreeStar) software version 10.0.7.

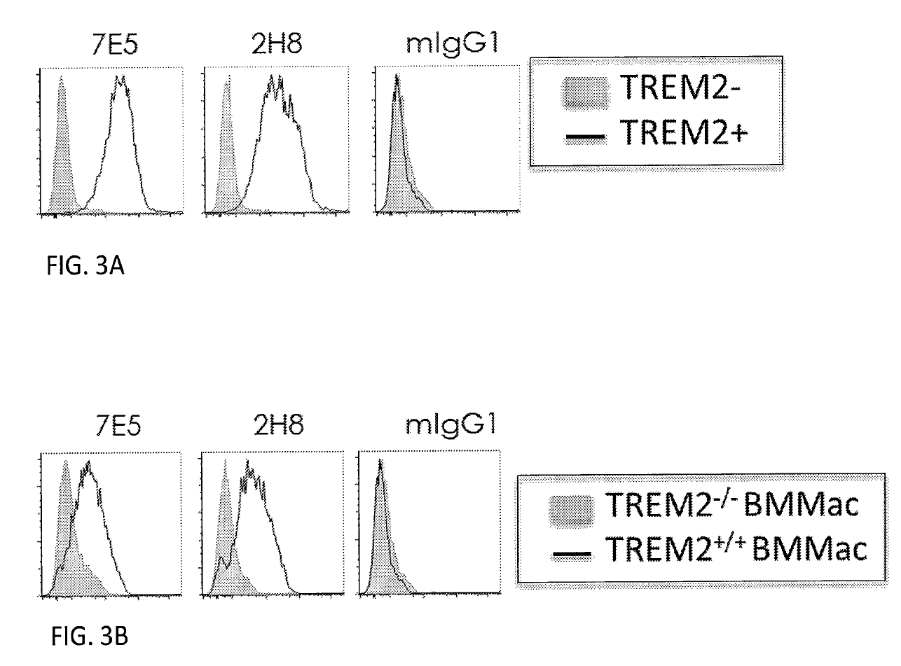

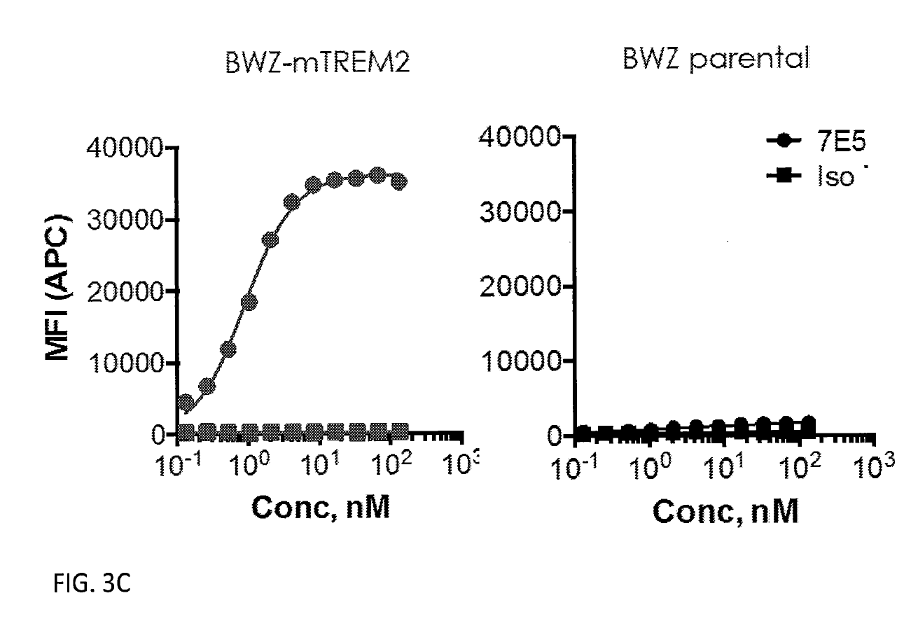

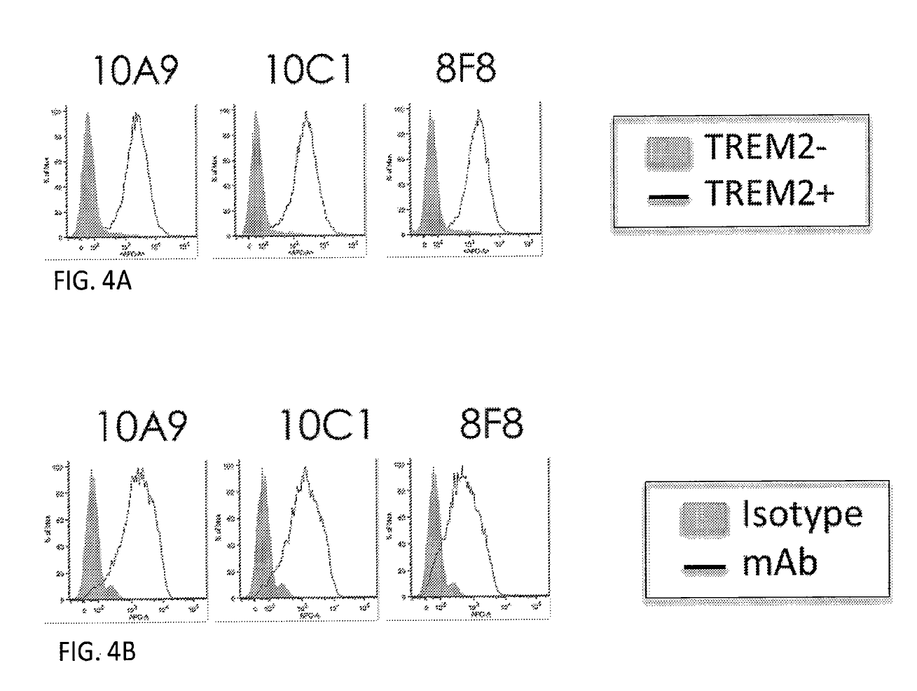

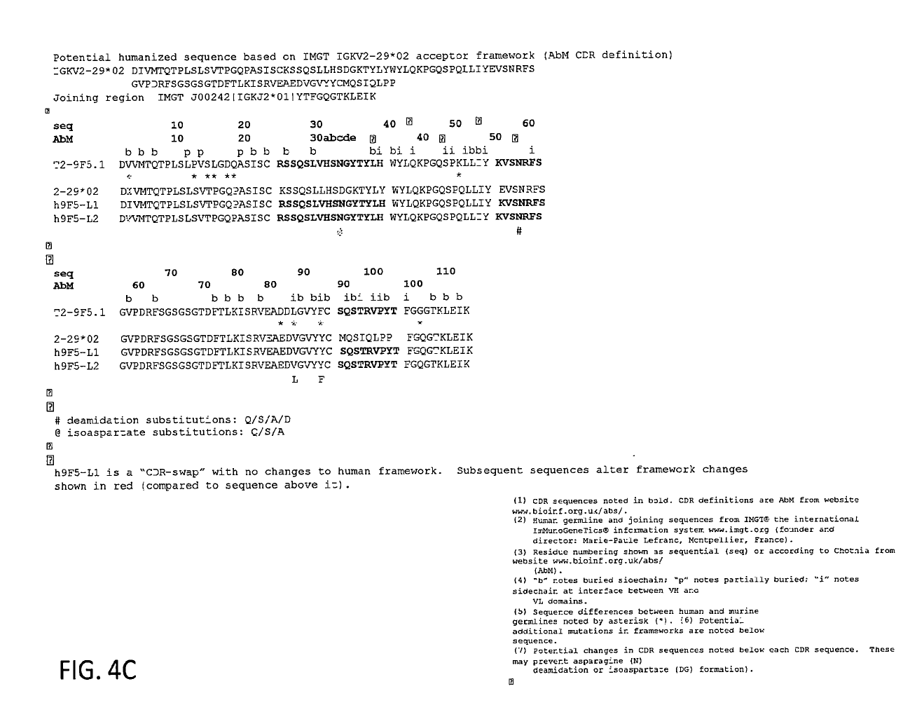

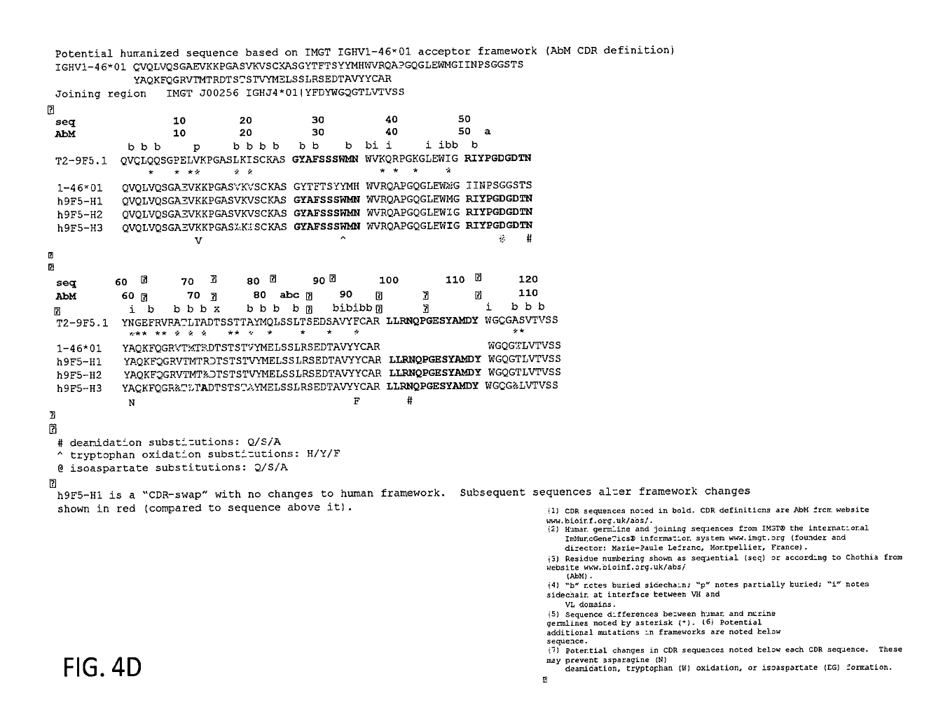

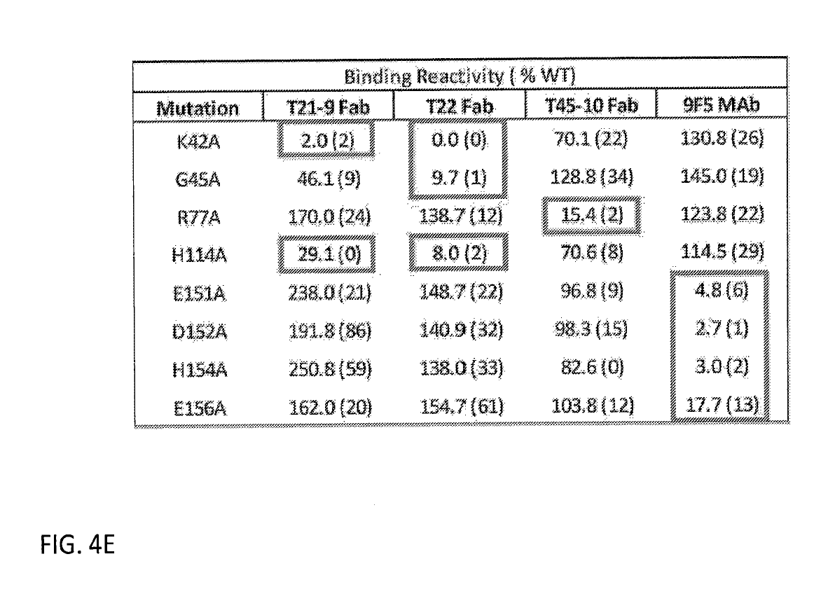

Antibodies 7E5 and 2H8, for example, demonstrated binding to a mouse cell line (BWZ T2) expressing recombinant mouse TREM2, as indicated by positive TREM2 antibody staining detected via FACS analysis (black outlined histograms) (FIG. 3A). The negative isotype control (antibody mIgG1) did not demonstrate binding. Antibodies 7E5 and 2H8 demonstrated antibody binding to WT (TREM+/+) bone marrow derived mouse macrophages (BMMac, mMac), but not to TREM2 deficient (TREM2−/−) mouse macrophages (BMMac, mMacs) (FIG. 3B). FIG. 3C shows a dose response curve demonstrating dose-dependent binding of the TREM2 antibody 7E5 to BWZ cells expressing recombinant mouse TREM2 but not to parental BWZ cells. Antibodies 10A9, 10C1, and 8F8 demonstrated binding to both a human cell line (293) expressing recombinant human TREM2 (FIG. 4A) and to primary human dendritic cells (hDC) (FIG. 4B).

Mean fluorescent intensities (MFI) values for mouse cell types bound by TREM2 antibodies 1H7, 2F6, 2H8, 3A7, 3B10, 7E5, 7F8, 8F8, and 11H5 are listed in Table 5. Binding is compared to the parental cell line (BWZ parental) and to BWZ cells that overexpress mouse TREM2 (BWZmT2). The table also depicts binding to primary mouse macrophages deficient in TREM2 (KO BMMACS), compared to wild-type primary macrophages (WT BMMACS). The results in Table 5 indicate that antibodies 1H7, 2F6, 2H8, 3A7, 3B10, 7E5, 7F8, 8F8, and 11H5 bind specifically to cell lines overexpressing mouse TREM2 on the cell membrane, but not to control cell lines that do not express TREM2. The antibodies also bind to mouse primary macrophages. Binding to mouse primary cells is specific, as it is not detected on primary cells derived from TREM2 KO mice or with the isotype control antibody mIgG1.

In Table 5, “mIgG1” refers to an isotype control antibody, “NT” refers to non-treated control, “2° Ab only” refers to a secondary antibody-only control, “RDT2” refers to a commercially available anti-TREM2 antibody (R&D Cat#F7E57291), and “ND” refers to not determined.

TABLE 5
Anti-TREM2 antibody binding to mouse cells

| | | MFI | % Positive | % Positive | K_{D} cell |
|---|---|---|---|---|---|
| | MFI | BWZ | WT | KO | binding |
| Antibody | BWZmT2 | parental | BMMACS | BMMACS | (nM) |
| 1H7 | 1963 | 76.9 | 40.4 | 0.961 | 3.53 |
| 2F6 | 1219 | 120 | 10.1 | 1.12 | 1.22 |
| 2H8 | 2668 | 66.2 | 36.5 | 1.13 | 6.86 |
| 3A7 | 2866 | 110 | 26.3 | 1.03 | 5.86 |
| 3B10 | 1309 | 123 | 7.22 | 0.735 | ND |
| 7E5 | 3038 | 127 | 27.6 | 3.97 | 10.38 |
| 7F8 | 1498 | 56.2 | 42.1 | 0.717 | 1.78 |
| 8F8 | 1644 | 53.5 | 6.42 | 1.09 | 6.90 |
| 11H5 | 1745 | 103 | 7.87 | 0.894 | 1.53 |
| mIgG1 | 278.00 | 125.00 | 0.574 | 0.725 | ND |
| NT | 245.00 | 134.00 | 0 | 0.0273 | ND |
| 2º Ab only | 123.00 | 68.00 | 0.523 | 0.836 | ND |
| RDT2 | ND | ND | 13.1 | 0.059 | ND |

Mean fluorescent intensities (MFI) values for human cell types bound by TREM2 antibodies 1A7, 3A2, 3B10, 6G12, 6H6, 7A9, 7B3, 8A1, 8E10, 8F11, 8F8, 9F5, 9G1, 9G3, 10A9, 10C1, 11A8, 12D9, 12E2, 12F9, and 12G6 are listed in Table 6. Binding is compared to the parental cell line (HEK parental) and to HEK cells that overexpress human TREM2 (HEKhT2). The table also depicts binding to primary human dendritic cells (hDC) and macrophages (hMAC). The results in Table 6 in indicate that antibodies 1A7, 3A2, 3B10, 6G12, 6H6, 7A9, 7B3, 8A1, 8E10, 8F11, 8F8, 9F5, 9G1, 9G3, 10A9, 10C1, 11A8, 12D9, 12E2, 12F9, and 12G6 bind specifically to cell lines overexpressing human TREM2 on the cell membrane, but not to control cell lines that do not express TREM2. The antibodies also bind to human primary dendritic cell s and macrophages. Binding to human primary cells is specific, as it is not detected with the isotype control antibodies mIgG1, mIgG2a, mIgG2b.

In Table 6, “Media” refers to a culture media only control, “2° Ab only” refers to a secondary antibody-only control, “mIgG1” refers to mouse IgG1 isotype control antibody, “mIgG2a” refers to mouse IgG2a isotype control antibody, “mIgG2b” refers to mouse IgG2b isotype control antibody, “mIgM” refers to mouse IgM isotype control antibody, “rIgG1” refers to rat IgG1 isotype control antibody, “RIgG2a” refers to rat IgG2a isotype control antibody, “RIgG2b” refers to rat IgG2b isotype control antibody, and “ND” refers to not determined.

TABLE 6
Anti-TREM2 antibody binding to human cells

| | | | | | K_{D} cell |
|---|---|---|---|---|---|
| | MFI | MFI | % Positive | % Positive | binding |
| Antibody | HEKhT2 | HEK parental | hDC | hMAC | (nM) |
| 1A7 | 4968 | 61 | 85 | 16 | 3.39 |
| 3A2 | 7297 | 74 | 77 | 18 | 4.98 |
| 3B10 | 2870 | 76 | 55 | 11 | 7.02 |
| 6G12 | 5484 | 50 | 82 | 19 | 4.11 |
| 6H6 | 13499 | 30 | 93 | 46 | 11.00 |
| 7A9 | 3316 | 31 | 59 | 12 | 6.56 |
| 7B3 | 4509 | 67 | 76 | 29 | 5.334 |
| 8A1 | 11013 | 18 | 94 | 34 | 4.637 |
| 8E10 | 9202 | 15 | 76 | 17 | 3.39 |
| 8F11 | 9668 | 32 | 79 | 15 | 2.905 |
| 8F8 | 3333 | 42 | 80 | 25 | 6.945 |
| 9F5 | 2911 | 56 | 73 | 26 | 0.96 |
| 9G1 | 3085 | 30 | 60 | 20 | 9.061 |
| 9G3 | 10677 | 22 | 78 | 30 | 12.21 |
| 10A9 | 11891 | 27 | 96 | 64 | 4.239 |
| 10C1 | 12956 | 70 | 92 | 46 | 8.904 |
| 11A8 | 2915 | 25 | 72 | 19 | 4.428 |
| 12E2 | 3616 | 20 | 63 | 13 | 12.71 |
| 12F9 | 2249 | 29 | 52 | 12 | 10.72 |
| 12G6 | 2521 | 39 | 73 | 23 | 4.224 |
| 2C7 | 4666 | 67 | 30 | 8 | ND |
| 2F5 | 948 | 65 | 6 | 2 | ND |
| 3C1 | 3560 | 55 | 30 | 13 | ND |
| 4D7 | 2418 | 132 | 17 | 8 | ND |
| 4D11 | 2222 | 41 | 16 | 10 | ND |
| 6C11 | 3279 | 55 | 21 | 3 | ND |
| 6G12 | 3288 | 135 | 15 | 7 | ND |
| 7A3 | 9573 | 98 | 33 | 3 | ND |
| 7C5 | 2812 | 77 | 15 | 13 | ND |
| 7E9 | 4553 | 60 | 35 | 12 | ND |
| 7F6 | 4265 | 107 | 31 | 7 | ND |
| 7G1 | 2262 | 99 | 11 | 1 | ND |
| 7H1 | 4556 | 100 | 11 | 8 | ND |
| 8C3 | 3631 | 47 | 32 | 3 | ND |
| 8F10 | 3460 | 102 | 9 | 5 | ND |
| 12A1 | 7599 | 72 | 26 | 3 | ND |
| 1E9 | 15076 | 107 | 75.5 | 16.8 | ND |
| 2C5 | 17811 | 247 | 56.4 | 11.0 | ND |
| 3C5 | 15696 | 127 | 85.8 | 29.1 | ND |
| 4C12 | 14671 | 158 | 84.2 | 26.1 | ND |
| 4F2 | 19264 | 116 | 85.3 | 25.2 | ND |
| 5A2 | 12642 | 187 | 68.0 | 19.4 | ND |
| 6B3 | 16697 | 102 | 81.1 | 14.4 | ND |
| 7D1 | 10742 | 121 | 69.9 | 19.3 | ND |
| 7D9 | 18659 | 150 | 93.9 | 43.3 | ND |
| 11D8 | 17254 | 121 | 69.7 | 20.1 | ND |
| 8A12 | 745 | 59.9 | 70.2 | 13.7 | ND |
| 10E7 | 3935 | 33.1 | 58.4 | 6.3 | ND |
| 10B11 | 14996 | 69.9 | 39.9 | 7.3 | ND |
| 10D2 | 6925 | 48 | 38.8 | 27.3 | ND |
| 7D5 | 2276 | 70.2 | 18.6 | 20.5 | ND |
| 2A7 | 8544 | 56.5 | 39.0 | 28.1 | ND |
| 3G12 | 1043 | 43.8 | 11.7 | 15.5 | ND |
| 6H9 | 6353 | 42.9 | 53.6 | 36.3 | ND |
| 8G9 | 4889 | 36.4 | 32.0 | 23.4 | ND |
| 9B4 | 6161 | 38.1 | 68.4 | 43.2 | ND |
| 10A1 | 2086 | 42.6 | 55.3 | 31.1 | ND |
| 11A8 | 1343 | 36.9 | 15.9 | 22.5 | ND |
| 12F3 | 7859 | 45.6 | 14.3 | 18.9 | ND |
| 2F8 | ND | ND | ND | ND | ND |
| 10E3 | ND | ND | ND | ND | ND |
| 1B4 | 8766 | 89.1 | 93.1 | ND | 6.2 |
| 6H2 | 13539 | 31 | 83.9 | ND | 5.4 |
| 7B11 | 12835 | 27.5 | 82.8 | ND | 3.7 |
| 18D8 | 7177 | 45.5 | 83.55 | ND | 3.7 |
| 18E4 | 978 | 21.3 | 15.4 | ND | 41.6 |
| 29F6 | 5851 | 29.7 | 94.55 | ND | 3.2 |
| 40D5 | 2136 | 38.9 | 74.2 | ND | 78.1 |
| 43B9 | 10082 | 238 | 76.8 | ND | 5.5 |
| 44A8 | 6912 | 25 | 17.1 | ND | 4.1 |
| 44B4 | 1409 | 24.4 | 89.05 | ND | 206.0 |
| 45D6 | 13888 | 22.2 | 81.1 | ND | 3.8 |
| 29F7 | 7755 | 43.8 | 85 | ND | 13.3 |
| 32G1 | 6018 | 70.7 | 95.35 | ND | 43.5 |
| Media | 22.6 | 86.3 | 0.6 | 0.7 | ND |
| 2º Ab only | 17.2 | 67.3 | 5.0 | 4.8 | ND |
| mIgG1 | 16.3 | 83.2 | 4.9 | 2.7 | ND |
| mIgG2a | 40.3 | 81.4 | 7.3 | 7.9 | ND |
| mIgG2b | 13 | 32.3 | 6.1 | 5.3 | ND |
| mIgM | 124 | 11.4 | 14.9 | 63.7 | ND |
| RIgG1 | 544 | 426 | 8.7 | 19.6 | ND |
| RIgG2a | 33 | 26.7 | 6.2 | 10.4 | ND |
| RIgG2b | 35 | 18.55 | 0.3 | 0.9 | ND |

Antibody Humanization

Antibody humanization is used to transform antibodies generated in a different species to best resemble a human antibody through sequence and structural relationships in order to prevent immunogenicity in human administration. Antibodies from different species share characteristic sequence and structural features that allow the grafting of the specificity-determining regions (SDRs) of the non-human antibody onto a human antibody framework. This results in retention of the specificity of the non-human antibody. The humanization process involves identification of the non-human antibody sequence and features, including the framework regions and SDRs. The following criteria are used to humanize an antibody: 1) percent similarity in framework regions between non-human and known human antibodies, 2) length similarity in SDRs between non-human and known human antibodies, 3) genes used to generate the framework regions of the human antibody, and 4) previous use of human antibody frameworks in humanizations and as therapeutics. Similarity in framework regions and SDR lengths are important because differences can generate structural differences in the antibody that can alter the specificity of the antibody. Specific genes used to generate the framework of human antibodies are known to be beneficial or detrimental to the stability or specificity of the antibody and are selectively used or avoided, accordingly. Lastly, previously successful humanization frameworks, including those used in human therapeutics, which are well tolerated with good half-lives, are likely candidates for future successful humanizations.

As shown in Tables 7A and 7B, humanized light chain and heavy variable region sequences were identified for each of the antibodies 4D11, 7C5, 6G12, 8F11, 8E10, 7E5, 7F8, 8F8, 1H7, 2H8, 3A2, 3A7, 3B10, 4F11, 6H6, 7A9, 7B3, 8A1, 9F5, 9G1, 9G3, 10A9, 11A8, 12D9, 12F9, 10C1, 7E9, and 8C1. In Tables 7A and 7B, bolded letters indicate CDR sequences.

TABLE 7A
Humanized light chain variable region sequences

| Antibody variant | Humanized sequences |
|---|---|
| Antibody 4D11 | Antibody 4D11 |
| 4D11V3-15 | EIVMTQSPATLSVSPGERATLSCRASENIYSFLAWYQQKPGQAPRLLIYNSKTF |
| | AEGIPARFSGSGSGFEFTLTISSLQSEDFAVYYCQHHYGTPPWTFGQGTKVEIK |
| | (SEQ ID NO: 220) |
| 4D11V1-9 | DIQLTQSPSFLSASVGDRVTITCRASENIYSFLAWYQQKPGKAPKLLIYNSKTF |
| | AEGVPSRFSGSGSGTEFTLTISSLQPEDFATYYCQHHYGTPPWTFGQGTKVEIK |
| | (SEQ ID NO: 221) |
| 4D11V3-11 | EIVLTQSPATLSLSPGERATLSCRASENIYSFLAWYQQKPGQAPRLLIYNSKTFA |
| | EGIPARFSGSGSGTDFTLTISSLEPEDFAVYYCQHHYGTPPWTFGQGTKVEIK |
| | (SEQ ID NO: 222) |
| 4D11V1-5 | DIQMTQSPSTLSASVGDRVTITCRASENIYSFLAWYQQKPGKAPKLLIYNSKTF |
| | AEGVPSRFSGSGSGTEFTLTISSLQPDDFATYYCQHHYGTPPWTFGQGTKVEIK |
| | (SEQ ID NO: 223) |
| 4D11V1-39 | DIQMTQSPSSLSASVGDRVTITCRASENIYSFLAWYQQKPGKAPKLLIYNSKTF |
| | AEGVPSRFSGSGSGTDFTLTISSLQPEDFATYYCQHHYGTPPWTFGQGTKVEIK |
| | (SEQ ID NO: 224) |
| 4D11V1-33 | DIQMTQSPSSLSASVGDRVTITCRASENIYSFLAWYQQKPGKAPKLLIYNSKTF |
| | AEGVPSRFSGSGSGTDFTFTISSLQPEDIATYYCQHHYGTPPWTFGQGTKVEIK |
| | (SEQ ID NO: 225) |
| 4D11V3-20 | EIVLTQSPGTLSLSPGERATLSCRASENIYSFLAWYQQKPGQAPRLLIYNSKTFA |
| | EGIPDRFSGSGSGTDFTLTISRLEPEDFAVYYCQHHYGTPPWTFGQGTKVEIK |
| | (SEQ ID NO: 226) |
| 4D11V2-28 | DIVMTQSPLSLPVTPGEPASISCRASENIYSFLAWYLQKPGQSPQLLIYNSKTFA |
| | EGVPDRFSGSGSGTDFTLKISRVEAEDVGVYYCQHHYGTPPWTFGQGTKVEIK |
| | (SEQ ID NO: 227) |
| 4D11V2-30 | DVVMTQSPLSLPVTLGQPASISCRASENIYSFLAWFQQRPGQSPRRLIYNSKTF |
| | AEGVPDRFSGSGSGTDFTLKISRVEAEDVGVYYCQHHYGTPPWTFGQGTKVEI |
| | K (SEQ ID NO: 228) |
| 4D11V4-1 | DIVMTQSPDSLAVSLGERATINCRASENIYSFLAWYQQKPGQPPKLLIYNSKTF |
| | AEGVPDRFSGSGSGTDFTLTISSLQAEDVAVYYCQHHYGTPPWTFGQGTKVEI |
| | K (SEQ ID NO: 229) |
| Antibody 7C5 | Antibody 7C5 |
| 7C5V3-15 | EIVMTQSPATLSVSPGERATLSCRASENIYSFLAWYQQKPGQAPRLLIYNSKTF |
| | AEGIPARFSGSGSGTEFTLTISSLQSEDFAVYYCQHHYGTPPWTFGQGTKVEIK |
| | (SEQ ID NO: 220) |
| 7C5V1-9 | DIQLTQSPSFLSASVGDRVTITCRASENIYSFLAWYQQKPGKAPKLLIYNSKTF |
| | AEGVPSRFSGSGSGTEFTLTISSLQPEDFATYYCQHHYGTPPWTFGQGTKVEIK |
| | (SEQ ID NO: 221) |
| 7C5V3-11 | EIVLTQSPATLSLSPGERATLSCRASENIYSFLAWYQQKPGQAPRLLIYNSKTFA |
| | EGIPARFSGSGSGTDFTLTISSLEPEDFAVYYCQHHYGTPPWTFGQGTKVEIK |
| | (SEQ ID NO: 222) |
| 7C5V1-5 | DIQMTQSPSTLSASVGDRVTITCRASENIYSFLAWYQQKPGKAPKLLIYNSKTF |
| | AEGVPSRFSGSGSGTEFTLTISSLQPDDFATYYCQHHYGTPPWTFGQGTKVEIK |
| | (SEQ ID NO: 223) |
| 7C5V1-39 | DIQMTQSPSSLSASVGDRVTITCRASENIYSFLAWYQQKPGKAPKLLIYNSKTF |
| | AEGVPSRFSGSGSGTDFTLTISSLQPEDFATYYCQHHYGTPPWTFGQGTKVEIK |
| | (SEQ ID NO: 224) |
| 7C5V1-33 | DIQMTQSPSSLSASVGDRVTITCRASENIYSFLAWYQQKPGKAPKLLIYNSKTF |
| | AEGVPSRFSGSGSGTDFTFTISSLQPEDIATYYCQHHYGTPPWTFGQGTKVEIK |
| | (SEQ ID NO: 225) |
| 7C5V3-20 | EIVLTQSPGTLSLSPGERATLSCRASENIYSFLAWYQQKPGQAPRLLIYNSKTFA |
| | EGIPDRFSGSGSGTDFTLTISRLEPEDFAVYYCQHHYGTPPWTFGQGTKVEIK |
| | (SEQ ID NO: 226) |
| 7C5V2-28 | DIVMTQSPLSLPVTPGEPASISCRASENIYSFLAWYLQKPGQSPQLLIYNSKTFA |
| | EGVPDRFSGSGSGTDFTLKISRVEAEDVGVYYCQHHYGTPPWTFGQGTKVEIK |
| | (SEQ ID NO: 227) |
| 7C5V2-30 | DVVMTQSPLSLPVTLGQPASISCRASENIYSFLAWFQQRPGQSPRRLIYNSKTF |
| | AEGVPDRFSGSGSGTDFTLKISRVEAEDVGVYYCQHHYGTPPWTFGQGTKVEI |
| | K (SEQ ID NO: 228) |
| 7C5V4-1 | DIVMTQSPDSLAVSLGERATINCRASENIYSFLAWYQQKPGQPPKLLIYNSKTF |
| | AEGVPDRFSGSGSGTDFTLTISSLQAEDVAVYYCQHHYGTPPWTFGQGTKVEI |
| | K (SEQ ID NO: 229) |
| Antibody 6G12 | Antibody 6G12 |
| 6G12V4-1 | DIVMTQSPDSLAVSLGERATINCKSSQSLLYSSNQKNCLAWYQQKPGQPPKLL |
| | IYWAFTRESGVPDRFSGSGSGTDFTLTISSLQAEDVAVYYCQQYYSYPLTFGQ |
| | GTKVEIK (SEQ ID NO: 231) |
| 6G12V2-30 | DVVMTQSPLSLPVTLGQPASISCKSSQSLLYSSNQKNCLAWFQQRPGQSPRRLI |
| | YWAFTRESGVPDRFSGSGSGTDFTLKISRVEAEDVGVYYCQQYYSYPLTFGQ |
| | GTKVEIK (SEQ ID NO: 232) |
| 6G12V2-28 | DIVMTQSPLSLPVTPGEPASISCKSSQSLLYSSNQKNCLAWYLQKPGQSPQLLI |
| | YWAFTRESGVPDRFSGSGSGTDFTLKISRVEAEDVGVYYCQQYYSYPLTFGQ |
| | GTKVEIK (SEQ ID NO: 233) |
| 6G12V1-9 | DIQLTQSPSFLSASVGDRVTITCKSSQSLLYSSNQKNCLAWYQQKPGKAPKLLI |
| | YWAFTRESGVPSRFSGSGSGTEFTLTISSLQPEDFATYYCQQYYSYPLTFGQGT |
| | KVEIK (SEQ ID NO: 234) |
| 6G12V1-5 | DIQMTQSPSTLSASVGDRVTITCKSSQSLLYSSNQKNCLAWYQQKPGKAPKLL |
| | IYWAFTRESGVPSRFSGSGSGTEFTLTISSLQPDDFATYYCQQYYSYPLTFGQG |
| | TKVEIK (SEQ ID NO: 235) |
| 6G12V3-15 | EIVMTQSPATLSVSPGERATLSCKSSQSLLYSSNQKNCLAWYQQKPGQAPRLLI |
| | YWAFTRESGIPARFSGSGSGTEFTLTISSLQSEDFAVYYCQQYYSYPLTFGQGT |
| | KVEIK (SEQ ID NO: 236) |
| 6G12V1-33 | DIQMTQSPSSLSASVGDRVTITCKSSQSLLYSSNQKNCLAWYQQKPGKAPKLLI |
| | YWAFTRESGVPSRFSGSGSGTDFTFTISSLQPEDIATYYCQQYYSYPLTFGQGT |
| | KVEIK (SEQ ID NO: 237) |
| 6G12V1-39 | DIQMTQSPSSLSASVGDRVTITCKSSQSLLYSSNQKNCLAWYQQKPGKAPKLLI |
| | YWAFTRESGVPSRFSGSGSGTDFTLTISSLQPEDFATYYCQQYYSYPLTFGQGT |
| | KVEIK (SEQ ID NO: 238) |
| 6G12V3-11 | EIVLTQSPATLSLSPGERATLSCKSSQSLLYSSNQKNCLAWYQQKPGQAPRLLI |
| | YWAFTRESGIPARFSGSGSGTDFTLTISSLEPEDFAVYYCQQYYSYPLTFGQGT |
| | KVEIK (SEQ ID NO: 239) |
| 6G12V3-20 | EIVLTQSPGTLSLSPGERATLSCKSSQSLLYSSNQKNCLAWYQQKPGQAPRLLI |
| | YWAFTRESGIPDRFSGSGSGTDFTLTISRLEPEDFAVYYCQQYYSYPLTFGQGT |
| | KVEIK (SEQ ID NO: 240) |
| Antibody 8F11 | Antibody 8F11 |
| 8F11V2-30 | DVVMTQSPLSLPVTLGQPASISCKSSQSLLYSNGKTFLSWFQQRPGQSPRRLIY |
| | LVSKLDSGVPDRFSGSGSGTDFTLKISRVEAEDVGVYYCMQGTHFPLTFGQGT |
| | KVEIK (SEQ ID NO: 242) |
| 8F11V2-28 | DIVMTQSPLSLPVTPGEPASISCKSSQSLLYSNGKTFLSWYLQKPGQSPQLLIYL |
| | VSKLDSGVPDRFSGSGSGTDFTLKISRVEAEDVGVYYCMQGTHFPLTFGQGT |
| | KVEIK (SEQ ID NO: 243) |
| 8F11V4-1 | DIVMTQSPDSLAVSLGERATINCKSSQSLLYSNGKTFLSWYQQKPGQPPKLLIY |
| | LVSKLDSGVPDRFSGSGSGTDFTLTISSLQAEDVAVYYCMQGTHFPLTFGQGT |
| | KVEIK (SEQ ID NO: 244) |
| 8F11V1-5 | DIQMTQSPSTLSASGDRVTITCKSSQSLLYSNGKTFLSWYQQKPGKAPKLLIY |
| | LVSKLDSGVPSRFSGSGSGTEFTLTISSLQPDDFATYYCMQGTHFPLTFGQGTK |
| | VEIK (SEQ ID NO: 245) |
| 8F11V1-9 | DIQLTQSPSFLSASVGDRVTITCKSSQSLLYSNGKTFLSWYQQKPGKAPKLLIY |
| | LVSKLDSGVPSRFSGSGSGTEFTLTISSLQPEDFATYYCMQGTHFPLTFGQGTK |
| | VEIK (SEQ ID NO: 246) |
| 8F11V1-39 | DIQMTQSPSSLSASVGDRVTITCKSSQSLLYSNGKTFLSWYQQKPGKAPKLLIY |
| | LVSKLDSGVPSRFSGSGSGTDFTLTISSLQPEDFATYYCMQGTHFPLTFGQGTK |
| | VEIK (SEQ ID NO: 247) |
| 8F11V1-33 | DIQMTQSPSSLSASVGDRVTITCKSSQSLLYSNGKTFLSWYQQKPGKAPKLLIY |
| | LVSKLDSGVPSRFSGSGSGTDFTFTISSLQPEDIATYYCMQGTHFPLTFGQGTK |
| | VEIK (SEQ ID NO: 248) |
| 8F11V3-15 | EIVMTQSPATLSVSPGERATLSCKSSQSLLYSNGKTFLSWYQQKPGQAPRLLIY |
| | LVSKLDSGIPARFSGSGSGTEFTLTISSLQSEDFAVYYCMQGTHFPLTFGQGTK |
| | VEIK (SEQ ID NO: 249) |
| 8F11V3-11 | EIVLTQSPATLSLSPGERATLSCKSSQSLLYSNGKTFLSWYQQKPGQAPRLLIY |
| | LVSKLDSGIPARFSGSGSGTDFTLTISSLEPEDFAVYYCMQGTHFPLTFGQGTK |
| | VEIK (SEQ ID NO: 250) |
| 8F11V3-20 | EIVLTQSPGTLSLSPGERATLSCKSSQSLLYSNGKTFLSWYQQKPGQAPRLLIY |
| | LVSKLDSGIPDRFSGSGSGTDFTLTISRLEPEDFAVYYCMQGTHFPLTFGQGTK |
| | VEIK (SEQ ID NO: 251) |
| Antibody 8E10 | Antibody 8E10 |
| 8E10V2-30 | DVVMTQSPLSLPVTLGQPASISCKSSQSLLDSDGKTYLNWFQQRPGQSPRRLIY |
| | LVSKLDSGVPDRFSGSGSGTDFTLKISRVEAEDVGVYYCWQGTHFPYTFGQG |
| | TKVEIK (SEQ ID NO: 253) |
| 8E10V2-28 | DIVMTQSPLSLPVTPGEPASISCKSSQSLLDSDGKTYLNWYLQKPGQSPQLLIY |
| | LVSKLDSGVPDRFSGSGSGTDFTLKISRVEAEDVGVYYCWQGTHFPYTFGQG |
| | TKVEIK (SEQ ID NO: 254) |
| 8E10V4-1 | DIVMTQSPDSLAVSLGERATINCKSSQSLLDSDGKTYLNWYQQKPGQPPKLLI |
| | YLVSKLDSGVPDRFSGSGSGTDFTLTISSLQAEDVAVYYCWQGTHFPYTFGQG |
| | TKVEIK (SEQ ID NO: 255) |
| 8E10V1-9 | DIQLTQSPSFLSASVGDRVTITCKSSQSLLDSDGKTYLNWYQQKPGKAPKLLIY |
| | LVSKLDSGVPSRFSGSGSGTEFTLTISSLQPEDFATYYCWQGTHFPYTFGQGTK |
| | VEIK (SEQ ID NO: 256) |
| 8E10V1-5 | DIQMTQSPSTLSASVGDRVTITCKSSQSLLDSDGKTYLNWYQQKPGKAPKLLI |
| | YLVSKLDSGVPSRFSGSGSGTEFTLTISSLQPDDFATYYCWQGTHFPYTFCTQGT |
| | KVEIK (SEQ ID NO: 257) |
| 8E10V1-39 | DIQMTQSPSSLSASVGDRVTITCKSSQSLLDSDGKTYLNWYQQKPGKAPKLLI |
| | YLVSKLDSGVPSRFSGSGSGTDFTLTISSLQPEDFATYYCWQGTHFPYTFGQGT |
| | KVEIK (SEQ ID NO: 258) |
| 8E10V1-33 | DIQMTQSPSSLSASVGDRVTITCKSSQSLLDSDGKTYLNWYQQKPGKAPKLLI |
| | YLVSKLDSGVPSRFSGSGSGTDFTFTISSLQPEDIATYYCWQGTHFPYTFGQGT |
| | KVEIK (SEQ ID NO: 259) |
| 8E10V3-11 | EIVLTQSPATLSLSPGERATLSCKSSQSLLDSDGKTYLNWYQQKPGQAPRLLIY |
| | LVSKLDSGIPARFSGSGSGTDFTLTISSLEPEDFAVYYCWQGTHFPYTFGQGTK |
| | VEIK (SEQ ID NO: 260) |
| 8E10V3-15 | EIVMTQSPATLSVSPGERATLSCKSSQSLLDSDGKTYLNWYQQKPGQAPRLLI |
| | YLVSKLDSGIPARFSGSGSGTEFTLTISSLQSEDFAVYYCWQGTHFPYTFGQGT |
| | KVEIK (SEQ ID NO: 261) |
| 8E10V3-20 | EIVLTQSPGTLSLSPGERATLSCKSSQSLLDSDGKTYLNWYQQKPGQAPRLLIY |
| | LVSKLDSGIPDRFSGSGSGTDFTLTISRLEPEDFAVYYCWQGTHFPYTFGQGTK |
| | VEIK (SEQ ID NO: 262) |
| Antibody 7E5 | Antibody 7E5 |
| 7E5V2-30 | DVVMTQSPLSLPVTLGQPASISCKSSQSLLYSNGKTFLSWFQQRPGQSPRRLIY |
| | LVSKLDSGVPDRFSGSGSGTDFTLKISRVEAEDVGVYYCMQGTHFPLTFGQGT |
| | KVEIK (SEQ ID NO: 242) |
| 7E5V2-28 | DIVMTQSPLSLPVTPGEPASISCKSSQSLLYSNGKTFLSWYLQKPGQSPQLLIYL |
| | VSKLDSGVPDRFSGSGSGTDFTLKISRVEAEDVGVYYCMQGTHFPLTFGQGT |
| | KVEIK (SEQ ID NO: 243) |
| 7E5V4-1 | DIVMTQSPDSLAVSLGERATINCKSSQSLLYSNGKTFLSWYQQKPGQPPKLLIY |
| | LVSKLDSGVPDRFSGSGSGTDFTLTISSLQAEDVAVYYCMQGTHFPLTFGQGT |
| | KVEIK (SEQ ID NO: 244) |
| 7E5V1-5 | DIQMTQSPSTLSASVGDRVTITCKSSQSLLYSNGKTFLSWYQQKPGKAPKLLIY |
| | LVSKLDSGVPSRFSGSGSGTEFTLTISSLQPDDFATYYCMQGTHFPLTFGQGTK |
| | VEIK (SEQ ID NO: 245) |
| 7E5V1-9 | DIQLTQSPSFLSASVGDRVTITCKSSQSLLYSNGKTFLSWYQQKPGKAPKLLIY |
| | LVSKLDSGVPSRFSGSGSGTEFTLTISSLQPEDFATYYCMQGTHFPLTFGQGTK |
| | VEIK (SEQ ID NO: 246) |
| 7E5V1-39 | DIQMTQSPSSLSASVGDRVTITCKSSQSLLYSNGKTFLSWYQQKPGKAPKLLIY |
| | LVSKLDSGVPSRFSGSGSGTDFTLTISSLQPEDFATYYCMQGTHFPLTFGQGTK |
| | VEIK (SEQ ID NO: 247) |
| 7E5V1-33 | DIQMTQSPSSLSASVGDRVTITCKSSQSLLYSNGKTFLSWYQQKPGKAPKLLIY |
| | LVSKLDSGVPSRFSGSGSGTDFTFTISSLQPEDIATYYCMQGTHFPLTFGQGTK |
| | VEIK (SEQ ID NO: 248) |
| 7E5V3-15 | EIVMTQSPATLSVSPGERATLSCKSSQSLLYSNGKTFLSWYQQKPGQAPRLLIY |
| | LVSKLDSGIPARFSGSGSGTEFTLTISSLQSEDFAVYYCMQGTHFPLTFGQGTK |
| | VEIK (SEQ ID NO: 249) |
| 7E5V3-11 | EIVLTQSPATLSLSPGERATLSCKSSQSLLYSNGKTFLSWYQQKPGQAPRLLIY |
| | LVSKLDSGIPARFSGSGSGTDFTLTISSLEPEDFAVYYCMQGTHFPLTFGQGTK |
| | VEIK (SEQ ID NO: 250) |
| 7E5V3-20 | EIVLTQSPGTLSLSPGERATLSCKSSQSLLYSNGKTFLSWYQQKPGQAPRLLIY |
| | LVSKLDSGIPDRFSGSGSGTDFTLTISRLEPEDFAVYYCMQGTHFPLTFGQGTK |
| | VEIK (SEQ ID NO: 251) |
| Antibody 7F8 | Antibody 7F8 |
| 7F8V3-11 | EIVLTQSPATLSLSPGERATLSCSASSSVSYMYWYQQKPGQAPRLLIYLTSILAS |
| | GIPARFSGSGSGTDFTLTISSLEPEDFAVYYCQQWSFNPYTFGQGTKVEIK (SEQ |
| | ID NO: 264) |
| 7F8V1-39 | DIQMTQSPSSLSASVGDRVTITCSASSSVSYMYWYQQKPGKAPKLLIYLTSILA |
| | SGVPSRFSGSGSGTDFTLTISSLQPEDFATYYCQQWSFNPYTFGQGTKVEIK |
| | (SEQ ID NO: 265) |
| 7F8V1-5 | DIQMTQSPSTLSASVGDRVTITCSASSSVSYMYWYQQKPGKAPKLLIYLTSILA |
| | SGVPSRFSGSGSGTEFTLTISSLQPDDFATYYCQQWSFNPYTFGQGTKVEIK |
| | (SEQ ID NO: 266) |
| 7F8V3-15 | EIVMTQSPATLSVSPGERATLSCSASSSVSYMYWYQQKPGQAPRLLIYLTSILA |
| | SGIPARFSGSGSGTEFTLTISSLQSEDFAVYYCQQWSFNPYTFGQGTKVEIK |
| | (SEQ ID NO: 267) |
| 7F8V1-9 | DIQLTQSPSFLSASVGDRVTITCSASSSVSYMYWYQQKPGKAPKLLIYLTSILAS |
| | GVPSRFSGSGSGTEFTLTISSLQPEDFATYYCQQWSFNPYTFGQGTKVEIK (SEQ |
| | ID NO: 268) |
| 7F8V1-33 | DIQMTQSPSSLSASVGDRVTITCSASSSVSYMYWYQQKPGKAPKLLIYLTSILA |
| | SGVPSRFSGSGSGTDFTFTISSLQPEDIATYYCQQWSFNPYTFGQGTKVEIK |
| | (SEQ ID NO: 269) |
| 7F8V3-20 | EIVLTQSPGTLSLSPGERATLSCSASSSVSYMYWYQQKPGQAPRLLIYLTSILAS |
| | GIPDRFSGSGSGTDFTLTISRLEPEDFAVYYCQQWSFNPYTFGQGTKVEIK (SEQ |
| | ID NO: 270) |
| 7F8V2-28 | DIVMTQSPLSLPVTPGEPASISCSASSSVSYMYWYLQKPGQSPQLLIYLTSILAS |
| | GVPDRFSGSGSGTDFTLKISRVEAEDVGVYYCQQWSFNPYTFGQGTKVEIK |
| | (SEQ ID NO: 271) |
| 7F8V2-30 | DVVMTQSPLSLPVTLGQPASISCSASSSVSYMYWFQQRPGQSPRRLIYLTSILAS |
| | GVPDRFSGSGSGTDFTLKISRVEAEDVGVYYCQQWSFNPYTFGQGTKVEIK |
| | (SEQ ID NO: 272) |
| 7F8V4-1 | DIVMTQSPDSLAVSLGERATINCSASSSVSYMYWYQQKPGQPPKLLIYLTSILA |
| | SGVPDRFSGSGSGTDFTLTISSLQAEDVAVYYCQQWSFNPYTFGQGTKVEIK |
| | (SEQ ID NO: 273) |
| Antibody 8F8 | Antibody 8F8 |
| 8F8V2-30 | DVVMTQSPLSLPVTLGQPASISCRSSQSLVHSNGNTYLHWFQQRPGQSPRRLIY |
| | KVSNRFSGVPDRFSGSGSGTDFTLKISRVEAEDVGVYYCSQSTHVPLTFGQGT |
| | KVEIK (SEQ ID NO: 275) |
| 8F8V2-28 | DIVMTQSPLSLPVTPGEPASISCRSSQSLVHSNGNTYLHWYLQKPGQSPQLLIY |
| | KVSNRFSGVPDRFSGSGSGTDFTLKISRVEAEDVGVYYCSQSTHVPLTFGQGT |
| | KVEIK (SEQ ID NO: 276) |
| 8F8V4-1 | DIVMTQSPDSLAVSLGERATINCRSSQSLVHSNGNTYLHWYQQKPGQPPKLLI |
| | YKVSNRFSGVPDRFSGSGSGTDFTLTISSLQAEDVAVYYCSQSTHVPLTFGQGT |
| | KVEIK (SEQ ID NO: 277) |
| 8F8V3-11 | EIVLTQSPATLSLSPGERATLSCRSSQSLVHSNGNTYLHWYQQKPGQAPRLLIY |
| | KVSNRFSGIPARFSGSGSGTDFTLTISSLEPEDFAVYYCSQSTHVPLTFGQGTKV |
| | EIK (SEQ ID NO: 278) |
| 8F8V1-39 | DIQMTQSPSSLSASVGDRVTITCRSSQSLVHSNGNTYLHWYQQKPGKAPKLLI |
| | YKVSNRFSGVPSRFSGSGSGTDFTLTISSLQPEDFATYYCSQSTHVPLTFGQGT |
| | KVEIK (SEQ ID NO: 279) |
| 8F8V1-33 | DIQMTQSPSSLSASVGDRVTITCRSSQSLVHSNGNTYLHWYQQKPGKAPKLLI |
| | YKVSNRFSGVPSRFSGSGSGTDFTFTISSLQPEDIATYYCSQSTHVPLTFGQGTK |
| | VEIK (SEQ ID NO: 280) |
| 8F8V3-15 | EIVMTQSPATLSVSPGERATLSCRSSQSLVHSNGNTYLHWYQQKPGQAPRLLI |
| | YKVSNRFSGIPARFSGSGSGTEFTLTISSLQSEDFAVYYCSQSTHVPLTFGQGTK |
| | VEIK (SEQ ID NO: 281) |
| 8F8V1-5 | DIQMTQSPSTLSASVGDRVTITCRSSQSLVHSNGNTYLHWYQQKPGKAPKLLI |
| | YKVSNRFSGVPSRFSGSGSGFEFTLTISSLQPDDFATYYCSQSTHVPLTFGQGT |
| | KVEIK (SEQ ID NO: 282) |
| 8F8V1-9 | DIQLTQSPSELSASVGDRVTITCRSSQSLVHSNGNTYLHWYQQKPGKAPKLLIY |
| | KVSNRFSGVPSRFSGSGSGTEFTLTISSLQPEDFATYYCSQSTHVPLTFGQGTKV |
| | EIK (SEQ ID NO: 283) |
| 8F8V3-20 | EIVLTQSPGTLSLSPGERATLSCRSSQSLVHSNGNTYLHWYQQKPGQAPRLLIY |
| | KVSNRFSGIPDRFSGSGSGTDFTLTISRLEPEDFAVYYCSQSTHVPLTFGQGTKV |
| | EIK (SEQ ID NO: 284) |
| Antibody1H7 | Antibody 1H7 |
| 1H7V1-39 | DIQMTQSPSSLSASVGDRVTITCSASSSVSYMYWYQQKPGKAPKLLIYLTSILA |
| | SGVPSRFSGSGSGTDFTLTISSLQPEDFATYYCQQWSFNPYTFGQGTKVEIK |
| | (SEQ ID NO: 265) |
| 1H7V3-11 | EIVLTQSPATLSLSPGERATLSCSASSSVSYMYWYQQKPGQAPRLLIYLTSILAS |
| | GIPARFSGSGSGTDFTLTISSLEPEDFAVYYCQQWSFNPYTFGQGTKVEIK (SEQ |
| | ID NO: 264) |
| 1H7V1-5 | DIQMTQSPSTLSASVGDRVTITCSASSSVSYMYWYQQKPGKAPKLLIYLTSILA |
| | SGVPSRFSGSGSGTEFTLTISSLQPDDFATYYCQQWSFNPYTFGQGTKVEIK |
| | (SEQ ID NO: 266) |
| 1H7V1-9 | DIQLTQSPSELSASVGDRVTITCSASSSVSYMYWYQQKPGKAPKLLIYLTSILAS |
| | GVPSRFSGSGSGTEFTLTISSLQPEDFATYYCQQWSFNPYTFGQGTKVEIK (SEQ |
| | ID NO: 268) |
| 1H7V3-15 | EIVMTQSPATLSVSPGERATLSCSASSSVSYMYWYQQKPGQAPRLLIYLTSILA |
| | SGIPARFSGSGSGTEFTLTISSLQSEDFAVYYCQQWSFNPYTFGQGTKVEIK |
| | (SEQ ID NO: 267) |
| 1H7V1-33 | DIQMTQSPSSLSASVGDRVTITCSASSSVSYMYWYQQKPGKAPKLLIYLTSILA |
| | SGVPSRFSGSGSGTDFTFTISSLQPEDIATYYCQQWSFNPYTFGQGTKVEIK |
| | (SEQ ID NO: 269) |
| 1H7V3-20 | EIVLTQSPGTLSLSPGERATLSCSASSSVSYMYWYQQKPGQAPRLLIYLTSILAS |
| | GIPDRFSGSGSGTDFTLTISRLEPEDFAVYYCQQWSFNPYTFGQGTKVEIK (SEQ |
| | ID NO: 270) |
| 1H7V2-28 | DIVMTQSPLSLPVTPGEPASISCSASSSVSYMYWYLQKPGQSPQLLIYLTSILAS |
| | GVPDRFSGSGSGTDFTLKISRVEAEDVGVYYCQQWSFNPYTFGQGTKVEIK |
| | (SEQ ID NO: 271) |
| 1H7V2-30 | DVVMTQSPLSLPVTLGQPASISCSASSSVSYMYWFQQRPGQSPRRLIYLTSILAS |
| | GVPDRFSGSGSGTDFTLKISRVEAEDVGVYYCQQWSFNPYTFGQGTKVEIK |
| | (SEQ ID NO: 272) |
| 1H7V4-1 | DIVMTQSPDSLAVSLGERATINCSASSSVSYMYWYQQKPGQPPKLLIYLTSILA |
| | SGVPDRFSGSGSGTDFTLTISSLQAEDVAVYYCQQWSFNPYTFGQGTKVEIK |
| | (SEQ ID NO: 273) |
| Antibody 2H8 | Antibody 2H8 |
| 2H8V3-11 | EIVLTQSPATLSLSPGERATLSCSASSSVSYMYWYQQKPGQAPRLLIYLTSILAS |
| | GIPARFSGSGSGTDFTLTISSLEPEDFAVYYCQQWSFNPYTFGQGTKVEIK (SEQ |
| | ID NO: 264) |
| 2H8V1-39 | DIQMTQSPSSLSASVGDRVTITCSASSSVSYMYWYQQKPGKAPKLLIYLTSILA |
| | SGVPSRFSGSGSGTDFTLTISSLQPEDFATYYCQQWSFNPYTFGQGTKVEIK |
| | (SEQ ID NO: 265) |
| 2H8V1-5 | DIQMTQSPSTLSASVGDRVTITCSASSSVSYMYWYQQKPGKAPKLLIYLTSILA |
| | SGVPSRFSGSGSGTEFTLTISSLQPDDFATYYCQQWSFNPYTFGQGTKVEIK |
| | (SEQ ID NO: 266) |
| 2H8V3-15 | EIVMTQSPATLSVSPGERATLSCSASSSVSYMYWYQQKPGQAPRLLIYLTSILA |
| | SGIPARFSGSGSGTEFTLTISSLQSEDFAVYYCQQWSFNPYTFGQGTKVEIK |
| | (SEQ ID NO: 267) |
| 2H8V1-9 | DIQLTQSPSELSASVGDRVTITCSASSSVSYMYWYQQKPGKAPKLLIYLTSILAS |
| | GVPSRFSGSGSGTEFTLTISSLQPEDFATYYCQQWSFNPYTFGQGTKVEIK (SEQ |
| | ID NO: 268) |
| 2H8V1-33 | DIQMTQSPSSLSASVGDRVTITCSASSSVSYMYWYQQKPGKAPKLLIYLTSILA |
| | SGVPSRFSGSGSGTDFTFTISSLQPEDIATYYCQQWSFNPYTFGQGTKVEIK |
| | (SEQ ID NO: 269) |
| 2H8V3-20 | EIVLTQSPGTLSLSPGERATLSCSASSSVSYMYWYQQKPGQAPRLLIYLTSILAS |
| | GIPDRFSGSGSGTDFTLTISRLEPEDFAVYYCQQWSFNPYTFGQGTKVEIK (SEQ |
| | ID NO: 270) |
| 2H8V2-28 | DIVMTQSPLSLPVTPGEPASISCSASSSVSYMYWYLQKPGQSPQLLIYLTSILAS |
| | GVPDRFSGSGSGTDFTLKISRVEAEDVGVYYCQQWSFNPYTFGQGTKVEIK |
| | (SEQ ID NO: 271) |
| 2H8V2-30 | DVVMTQSPLSLPVTLGQPASISCSASSSVSYMYWFQQRPGQSPRRLIYLTSILAS |
| | GVPDRFSGSGSGTDFTLKISRVEAEDVGVYYCQQWSFNPYTFGQGTKVEIK |
| | (SEQ ID NO: 272) |
| 2H8V4-1 | DIVMTQSPDSLAVSLGERATINCSASSSVSYMYWYQQKPGQPPKLLIYLTSILA |
| | SGVPDRFSGSGSGTDFTLTISSLQAEDVAVYYCQQWSFNPYTFGQGTKVEIK |
| | (SEQ ID NO: 273) |
| Antibody 3A2 | Antibody 3A2 |
| 3A2 V2-30 | DVVMTQSPLSLPVTLGQPASISCRSSQTIIHSNGNTYLEWFQQRPGQSPRRLIY |
| | KVSNRFSGVPDRFSGSGSGTDFTLKISRVEAEDVGVYYCFQGSHVPYTFGQGT |
| | KVEIK (SEQ ID NO: 288) |
| 3A2 V2-28 | DIVMTQSPLSLPVTPGEPASISCRSSQTIIHSNGNTYLEWYLQKPGQSPQLLIYK |
| | VSNRFSGVPDRFSGSGSGTDFTLKISRVEAEDVGVYYCFQGSHVPYTFGQGTK |
| | VEIK (SEQ ID NO: 289) |
| 3A2 V4-1 | DIVMTQSPDSLAVSLGERATINCRSSQTIIHSNGNTYLEWYQQKPGQPPKLLIY |
| | KVSNRFSGVPDRFSGSGSGTDFTLTISSLQAEDVAVYYCFQGSHVPYTFGQGT |
| | KVEIK (SEQ ID NO: 290) |
| 3A2 V3-11 | EIVLTQSPATLSLSPGERATLSCRSSQTIIHSNGNTYLEWYQQKPGQAPRLLIYK |
| | VSNRFSGIPARFSGSGSGTDFTLTISSLEPEDFAVYYCFQGSHVPYTFGQGTKVE |
| | IK (SEQ ID NO: 291) |
| 3A2 V1-9 | DIQLTQSPSFLSASVGDRVTITCRSSQTIIHSNGNTYLEWYQQKPGKAPKLLIY |
| | KVSNRFSGVPSRFSGSGSGTEFTLTISSLQPEDFATYYCFQGSHVPYTFGQGTK |
| | VEIK (SEQ ID NO: 292) |
| 3A2 V1-33 | DIQMTQSPSSLSASVGDRVTITCRSSQTIIHSNGNTYLEWYQQKPGKAPKLLIY |
| | KVSNRFSGVPSRFSGSGSGTDFTFTISSLQPEDIATYYCFQGSHVPYTFGQGTK |
| | VEIK (SEQ ID NO: 293) |
| 3A2 V1-39 | DIQMTQSPSSLSASVGDRVTITCRSSQTIIHSNGNTYLEWYQQKPGKAPKLLIY |
| | KVSNRFSGVPSRFSGSGSGTDFTLTISSLQPEDFATYYCFQGSHVPYTFGQGTK |
| | VEIK (SEQ ID NO: 294) |
| 3A2 V3-15 | EIVMTQSPATLSVSPGERATLSCRSSQTIIHSNGNTYLEWYQQKPGQAPRLLIY |
| | KVSNRFSGIPARFSGSGSGFEFTLTISSLQSEDFAVYYCFQGSHVPYTFGQGTK |
| | VEIK (SEQ ID NO: 295) |
| 3A2 V1-5 | DIQMTQSSTLSASVGDRVTITCRSSQTIIHSNGNTYLEWYQQKPGKAPKLLIY |
| | KVSNRFSGVPSRFSGSGSGTEFTLTISSLQPDDFATYYCFQGSHVPYTFGQGTK |
| | VEIK (SEQ ID NO: 296) |
| 3A2 V3-20 | EIVLTQSPGTLSLSPGERATLSCRSSQTIIHSNGNTYLEWYQQKPGQAPRLLIYK |
| | VSNRFSGIPDRFSGSGSGTDFTLTISRLEPEDFAVYYCFQGSHVPYTFGQGTKV |
| | EIK (SEQ ID NO: 297) |
| Antibody 3A7 | Antibody 3A7 |
| 3A7 V2-30 | DVVMTQSPLSLPVTLGQPASISCKSSQSLLYSNGKTFLSWFQQRPGQSPRRLIY |
| | LVSKLDSGVPDRFSGSGSGTDFTLKISRVEAEDVGVYYCMQGTHFPLTFGQGT |
| | KVEIK (SEQ ID NO: 242) |
| 3A7 V2-28 | DIVMTQSPLSLPVTPGEPASISCKSSQSLLYSNGKTFLSWYLQKPGQSPQLLIYL |
| | VSKLDSGVPDRFSGSGSGTDFTLKISRVEAEDVGVYYCMQGTHFPLTFGQGT |
| | KVEIK (SEQ ID NO: 243) |
| 3A7 V4-1 | DIVMTQSPDSLAVSLGERATINCKSSQSLLYSNGKTFLSWYQQKPGQPPKLLIY |
| | LVSKLDSGVPDRFSGSGSGTDFTLTISSLQAEDVAVYYCMQGTHFPLTFGQGT |
| | KVEIK (SEQ ID NO: 244) |
| 3A7 V1-39 | DIQMTQSPSSLSASVGDRVTITCKSSQSLLYSNGKTFLSWYQQKPGKAPKLLIY |
| | LVSKLDSGVPSRFSGSGSGTDFTLTISSLQPEDFATYYCMQGTHFPLTFGQGTK |
| | VEIK (SEQ ID NO: 247) |
| 3A7 V1-9 | DIQLTQSPSFLSASVGDRVTITCKSSQSLLYSNGKTFLSWYQQKPGKAPKLLIY |
| | LVSKLDSGVPSRFSGSGSGTEFTLTISSLQPEDFATYYCMQGTHFPLTFGQGTK |
| | VEIK (SEQ ID NO: 246) |
| 3A7 V1-5 | DIQMTQSPSTLSASVGDRVTITCKSSQSLLYSNGKTFLSWYQQKPGKAPKLLIY |
| | LVSKLDSGVPSRFSGSGSGTEFTLTISSLQPDDFATYYCMQGTHFPLTFGQGTK |
| | VEIK (SEQ ID NO: 245) |
| 3A7 V1-33 | DIQMTQSPSSLSASVGDRVTITCKSSQSLLYSNGKTFLSWYQQKPGKAPKLLIY |
| | LVSKLDSGVPSRFSGSGSGTDFTFTISSLQPEDIATYYCMQGTHFPLTFGQGTK |
| | VEIK (SEQ ID NO: 248) |
| 3A7 V3-15 | EIVMTQSPATLSVSPGERATLSCKSSQSLLYSNGKTFLSWYQQKPGQAPRLLIY |
| | LVSKLDSGIPARFSGSGSGTEFTLTISSLQSEDFAVYYCMQGTHFPLTFGQGTK |
| | VEIK (SEQ ID NO: 249) |
| 3A7 V3-11 | EIVLTQSPATLSLSPGERATLSCKSSQSLLYSNGKTFLSWYQQKPGQAPRLLIY |
| | LVSKLDSGIPARFSGSGSGTDFTLTISSLEPEDFAVYYCMQGTHFPLTFGQGTK |
| | VEIK (SEQ ID NO: 250) |
| 3A7 V3-20 | EIVLTQSPGTLSLSPGERATLSCKSSQSLLYSNGKTFLSWYQQKPGQAPRLLIY |
| | LVSKLDSGIPDRFSGSGSGTDFTLTISRLEPEDFAVYYCMQGTHFPLTFGQGTK |
| | VEIK (SEQ ID NO: 251) |
| Antibody 3B10 | Antibody 3B10 |
| 3B10V4-1 | DIVMTQSPDSLAVSLGERATINCKSSQSLLYSSDQKNYLAWYQQKPGQPPKLL |
| | IYWASTRESGVPDRFSGSGSGTDFTLTISSLQAEDVAVYYCQQYYSYPLTFGQ |
| | GTKVEIK (SEQ ID NO: 300) |
| 3B10V2-28 | DIVMTQSPLSLPVTPGEPASISCKSSQSLLYSSDQKNYLAWYLQKPGQSPQLLI |
| | YWASTRESGVPDRFSGSGSGTDFTLKISRVEAEDVGVYYCQQYYSYPLTFGQ |
| | GTKVEIK (SEQ ID NO: 301) |
| 3B10V2-30 | DVVMTQSPLSLPVTLGQPASISCKSSQSLLYSSDQKNYLAWFQQRPGQSPRRLI |
| | YWASTRESGVPDRFSGSGSGTDFTLKISRVEAEDVGVYYCQQYYSYPLTFGQ |
| | GTKVEIK (SEQ ID NO: 302) |
| 3B10V1-5 | DIQMTQSPSTLSASVGDRVTITCKSSQSLLYSSDQKNYLAWYQQKPGKAPKLL |
| | IYWASTRESGVPSRFSGSGSGTEFTLTISSLQPDDFATYYCQQYYSYPLTFGQG |
| | TKVEIK (SEQ ID NO: 303) |
| 3B10V1-9 | DIQLTQSPSFLSASVGDRVTITCKSSQSLLYSSDQKNYLAWYQQKPGKAPKLLI |
| | YWASTRESGVPSRFSGSGSGTEFTLTISSLQPEDFATYYCQQYYSYPLTFGQGT |
| | KVEIK (SEQ ID NO: 304) |
| 3B10V3-15 | EIVMTQSPATLSVSPGERATLSCKSSQSLLYSSDQKNYLAWYQQKPGQAPRLLI |
| | YWASTRESGIPARFSGSGSGTEFTLTISSLQSEDFAVYYCQQYYSYPLTFGQGT |
| | KVEIK (SEQ ID NO: 305) |
| 3B10V1-39 | DIQMTQSPSSLSASVGDRVTITCKSSQSLLYSSDQKNYLAWYQQKPGKAPKLLI |
| | YWASTRESGVPSRFSGSGSGTDFTLTISSLQPEDFATYYCQQYYSYPLTFGQGT |
| | KVEIK (SEQ ID NO: 306) |
| 3B10V3-11 | EIVLTQSPATLSLSPGERATLSCKSSQSLLYSSDQKNYLAWYQQKPGQAPRLLI |
| | YWASTRESGIPARFSGSGSGTDFTLTISSLEPEDFAVYYCQQYYSYPLTFGQGT |
| | KVEIK (SEQ ID NO: 307) |
| 3B10V1-33 | DIQMTQSPSSLSASVGDRVTITCKSSQSLLYSSDQKNYLAWYQQKPGKAPKLLI |
| | YWASTRESGVPSRFSGSGSGTDFTFTISSLQPEDIATYYCQQYYSYPLTFGQGT |
| | KVEIK (SEQ ID NO: 308) |
| 3B10V3-20 | EIVLTQSPGTLSLSPGERATLSCKSSQSLLYSSDQKNYLAWYQQKPGQAPRLLI |
| | YWASTRESGIPDRFSGSGSGTDFTLTISRLEPEDFAVYYCQQYYSYPLTFGQGT |
| | KVEIK (SEQ ID NO: 309) |
| Antibody 4F11 | Antibody 4F11 |
| 4F11V2-30 | DVVMTQSPLSLPVTLGQPASISCRSSQTIIHSNGNTYLEWFQQRPGQSPRRLIY |
| | KVSNRFSGVPDRFSGSGSGTDFTLKISRVEAEDVGVYYCFQGSHVPYTFGQGT |
| | KVEIK (SEQ ID NO: 288) |
| 4F11V2-28 | DIVMTQSPLSLPVTPGEPASISCRSSQTIIHSNGNTYLEWYLQKPGQSPQLLIYK |
| | VSNRFSGVPDRFSGSGSGTDFTLKISRVEAEDVGVYYCFQGSHVPYTFGQGTK |
| | VEIK (SEQ ID NO: 289) |
| 4F11V4-1 | DIVMTQSPDSLAVSLGERATINCRSSQTIIHSNGNTYLEWYQQKPGQPPKLLIY |
| | KVSNRFSGVPDRFSGSGSGTDFTLTISSLQAEDVAVYYCFQGSHVPYTFGQGT |
| | KVEIK (SEQ ID NO: 290) |
| 4F11V3-11 | EIVLTQSPATLSLSPGERATLSCRSSQTIIHSNGNTYLEWYQQKPGQAPRLLIYK |
| | VSNRFSGIPARFSGSGSGTDFTLTISSLEPEDFAVYYCFQGSHVPYTFGQGTKVE |
| | IK (SEQ ID NO: 291) |
| 4F11V3-15 | EIVMTQSPATLSVSPGERATLSCRSSQTIIHSNGNTYLEWYQQKPGQAPRLLIY |
| | KVSNRFSGIPARFSGSGSGTEFTLTISSLQSEDFAVYYCFQGSHVPYTFGQGTK |
| | VEIK (SEQ ID NO: 295) |
| 4F11V1-33 | DIQMTQSPSSLSASVGDRVTITCRSSQTIIHSNGNTYLEWYQQKPGKAPKLLIY |
| | KVSNRFSGVPSRFSGSGSGTDFTFTISSLQPEDIATYYCFQGSHVPYTFGQGTK |
| | VEIK (SEQ ID NO: 293) |
| 4F11V1-39 | DIQMTQSPSSLSASVGDRVTITCRSSQTIIHSNGNTYLEWYQQKPGKAPKLLIY |
| | KVSNRFSGVPSRFSGSGSGTDFTLTISSLQPEDFATYYCFQGSHVPYTFGQGTK |
| | VEIK (SEQ ID NO: 294) |
| 4F11V1-9 | DIQLTQSPSFLSASVGDRVTITCRSSQTIIHSNGNTYLEWYQQKPGKAPKLLIY |
| | KVSNRFSGVPSRFSGSGSGTEFTLTISSLQPEDFATYYCFQGSHVPYTFGQGTK |
| | VEIK (SEQ ID NO: 292) |
| 4F11V1-5 | DIQMTQSPSTLSASVGDRVTITCRSSQTIIHSNGNTYLEWYQQKPGKAPKLLIY |
| | KVSNRFSGVPSRFSGSGSGTEFTLTISSLQPDDFATYYCFQGSHVPYTFGQGTK |
| | VEIK (SEQ ID NO: 296) |
| 4F11V3-20 | EIVLTQSPGTLSLSPGERATLSCRSSQTIIHSNGNTYLEWYQQKPGQAPRLLIYK |
| | VSNRFSGIPDRFSGSGSGTDFTLTISRLEPEDFAVYYCFQGSHVPYTFGQGTKV |
| | EIK (SEQ ID NO: 297) |
| Antibody 6H6 | Antibody 6H6 |
| 6H6V4-1 | DIVMTQSPDSLAVSLGERATINCKSSQSVFYSSNQKNYLAWYQQKPGQPPKLL |
| | IYWASTRESGVPDRFSGSGSGTDFTLTISSLQAEDVAVYYCHQYLSSLTFGQGT |
| | KVEIK (SEQ ID NO: 680) |
| 6H6V2-28 | DIVMTQSPLSLPVTPGEPASISCKSSQSVFYSSNQKNYLAWYLQKPGQSPQLLI |
| | YWASTRESGVPDRFSGSGSGTDFTLKISRVEAEDVGVYYCHQYLSSLTFGQGT |
| | KVEIK (SEQ ID NO: 681) |
| 6H6V2-30 | DVVMTQSPLSLPVTLGQPASISCKSSQSVFYSSNQKNYLAWFQQRPGQSPRRLI |
| | YWASTRESGVPDRFSGSGSGTDFTLKISRVEAEDVGVYYCHQYLSSLTFGQGT |
| | KVEIK (SEQ ID NO: 682) |
| 6H6V1-5 | DIQMTQSPSTLSASVGDRVTITCKSSQSVFYSSNQKNYLAWYQQKPGKAPKLL |
| | IYWASTRESGVPSRFSGSGSGTEFTLTISSLQPDDFATYYCHQYLSSLTFGQGT |
| | KVEIK (SEQ ID NO: 683) |
| 6H6V1-9 | DIQLTQSPSFLSASVGDRVTITCKSSQSVFYSSNQKNYLAWYQQKPGKAPKLLI |
| | YWASTRESGVPSRFSGSGSGTEFTLTISSLQPEDFATYYCHQYLSSLTFGQGTK |
| | VEIK (SEQ ID NO: 684) |
| 6H6V3-15 | EIVMTQSPATLSVSPGERATLSCKSSQSVFYSSNQKNYLAWYQQKPGQAPRLLI |
| | YWASTRESGIPARFSGSGSGTEFTLTISSLQSEDFAVYYCHQYLSSLTFGQGTK |
| | VEIK (SEQ ID NO: 685) |
| 6H6V1-33 | DIQMTQSPSSLSASVGDRVTITCKSSQSVFYSSNQKNYLAWYQQKPGKAPKLLI |
| | YWASTRESGVPSRFSGSGSGTDFTFTISSLQPEDIATYYCHQYLSSLTFGQGTK |
| | VEIK (SEQ ID NO: 686) |
| 6H6V3-11 | EIVLTQ SPATLSLSPGERATLSCKSSQSVFYSSNQKNYLAWYQQKPGQAPRLLI |
| | YWASTRESGIPARFSGSGSGTDFTLTISSLEPEDFAVYYCHQYLSSLTFGQGTK |
| | VEIK (SEQ ID NO: 687) |
| 6H6V1-39 | DIQMTQSPSSLSASVGDRVTITCKSSQSVFYSSNQKNYLAWYQQKPGKAPKLLI |
| | YWASTRESGVPSRFSGSGSGTDFTLTISSLQPEDFATYYCHQYLSSLTFGQGTK |
| | VEIK (SEQ ID NO: 688) |
| 6H6V3-20 | EIVLTQ SPGTLSLSPGERATLSCKSSQSVFYSSNQKNYLAWYQQKPGQAPRLLI |
| | YWASTRESGIPDRFSGSGSGTDFTLTISRLEPEDFAVYYCHQYLSSLTFGQGTK |
| | VEIK (SEQ ID NO: 689) |
| Antibody 7A9 | Antibody 7A9 |
| 7A9V1-9 | DIQLTQSPSFLSASVGDRVTITCRASENIYSYLAWYQQKPGKAPKLLIYKAKTL |
| | AEGVPSRFSGSGSGTEFTLTISSLQPEDFATYYCQHHYGTPFTFGQGTKVEIK |
| | (SEQ ID NO: 312) |
| 7A9V3-11 | EIVLTQSPATLSLSPGERATLSCRASENIYSYLAWYQQKPGQAPRLLIYKAKTL |
| | AEGIPARFSGSGSGTDFTLTISSLEPEDFAVYYCQHHYGTPFTFGQGTKVEIK |
| | (SEQ ID NO: 313) |
| 7A9V1-5 | DIQMTQSPSTLSASVGDRVTITCRASENIYSYLAWYQQKPGKAPKLLIYKAKT |
| | LAEGVPSRFSGSGSGTEFTLTISSLQPDDFATYYCQHHYGTPFTFGQGTKVEIK |
| | (SEQ ID NO: 314) |
| 7A9V3-15 | EIVMTQSPATLSVSPGERATLSCRASENIYSYLAWYQQKPGQAPRLLIYKAKTL |
| | AEGIPARFSGSGSGTEFTLTISSLQSEDFAVYYCQHHYGTPFTFGQGTKVEIK |
| | (SEQ ID NO: 315) |
| 7A9V1-39 | DIQMTQSPSSLSASVGDRVTITCRASENIYSYLAWYQQKPGKAPKLLIYKAKTL |
| | AEGVPSRFSGSGSGTDFTLTISSLQPEDFATYYCQHHYGTPFTFGQGTKVEIK |
| | (SEQ ID NO: 316) |
| 7A9V1-33 | DIQMTQSPSSLSASVGDRVTITCRASENIYSYLAWYQQKPGKAPKLLIYKAKTL |
| | AEGVPSRFSGSGSGTDFTFTISSLQPEDIATYYCQHHYGTPFTFGQGTKVEIK |
| | (SEQ ID NO: 317) |
| 7A9V3-20 | EIVLTQSPGTLSLSPGERATLSCRASENIYSYLAWYQQKPGQAPRLLIYKAKTL |
| | AEGIPDRFSGSGSGTDFTLTISRLEPEDFAVYYCQHHYGTPFTFGQGTKVEIK |
| | (SEQ ID NO: 318) |
| 7A9V2-28 | DIVMTQSPLSLPVTPGEPASISCRASENIYSYLAWYLQKPGQSPQLLIYKAKTL |
| | AEGVPDRFSGSGSGTDFTLKISRVEAEDVGVYYCQHHYGTPFTFGQGTKVEIK |
| | (SEQ ID NO: 319) |
| 7A9V4-1 | DIVMTQSPDSLAVSLGERATINCRASENIYSYLAWYQQKPGQPPKLLIYKAKT |
| | LAEGVPDRFSGSGSGTDFTLTISSLQAEDVAVYYCQHHYGTPFTFGQGTKVEI |
| | K (SEQ ID NO: 320) |
| 7A9V2-30 | DVVMTQSPLSLPVTLGQPASISCRASENIYSYLAWFQQRPGQSPRRLIYKAKTL |
| | AEGVPDRFSGSGSGTDFTLKISRVEAEDVGVYYCQHHYGTPFTFGQGTKVEIK |
| | (SEQ ID NO: 321) |
| Antibody 8A1 | Antibody 8A1 |
| 8A1V3-15 | EIVMTQSPATLSVSPGERATLSCRTSENVYSNLAWYQQKPGQAPRLLIYAATN |
| | LADGIPARFSGSGSGTEFTLTISSLQSEDFAVYYCHHFWGTPYTFGQGTKVEIK |
| | (SEQ ID NO: 323) |
| 8A1V3-11 | EIVLTQSPATLSLSPGERATLSCRTSENVYSNLAWYQQKPGQAPRLLIYAATNL |
| | ADGIPARFSGSGSGTDFTLTISSLEPEDFAVYYCHHFWGTPYTFGQGTKVEIK |
| | (SEQ ID NO: 324) |
| 8A1V1-9 | DIQLTQSPSELSASVGDRVTITCRTSENVYSNLAWYQQKPGKAPKLLIYAATNL |
| | ADGVPSRFSGSGSGTEFTLTISSLQPEDFATYYCHHFWGTPYTFGQGTKVEIK |
| | (SEQ ID NO: 325) |
| 8A1V1-5 | DIQMTQSPSTLSASVGDRVTITCRTSENVYSNLAWYQQKPGKAPKLLIYAATN |
| | LADGVPSRFSGSGSGTEFTLTISSLQPDDFATYYCHHFWGTPYTFGQGTKVEIK |
| | (SEQ ID NO: 326) |
| 8A1V1-39 | DIQMTQSPSSLSASVGDRVTITCRTSENVYSNLAWYQQKPGKAPKLLIYAATN |
| | LADGVPSRFSGSGSGTDFTLTISSLQPEDFATYYCHHFWGTPYTFGQGTKVEIK |
| | (SEQ ID NO: 327) |
| 8A1V1-33 | DIQMTQSPSSLSASVGDRVTITCRTSENVYSNLAWYQQKPGKAPKLLIYAATN |
| | LADGVPSRFSGSGSGTDFTFTISSLQPEDIATYYCHHFWGTPYTFGQGTKVEIK |
| | (SEQ ID NO: 328) |
| 8A1V3-20 | EIVLTQSPGTLSLSPGERATLSCRTSENVYSNLAWYQQKPGQAPRLLIYAATNL |
| | ADGIPDRFSGSGSGTDFTLTISRLEPEDFAVYYCHHFWGTPYTFGQGTKVEIK |
| | (SEQ ID NO: 329) |
| 8A1V2-28 | DIVMTQSPLSLPVTPGEPASISCRTSENVYSNLAWYLQKPGQSPQLLIYAATNL |
| | ADGVPDRFSGSGSGTDFTLKISRVEAEDVGVYYCHHFWGTPYTFGQGTKVEIK |
| | (SEQ ID NO: 330) |
| 8A1V2-30 | DVVMTQSPLSLPVTLGQPASISCRTSENVYSNLAWFQQRPGQSPRRLIYAATNL |
| | ADGVPDRFSGSGSGTDFTLKISRVEAEDVGVYYCHHFWGTPYTFGQGTKVEIK |
| | (SEQ ID NO: 331) |
| 8A1V4-1 | DIVMTQSPDSLAVSLGERATINCRTSENVYSNLAWYQQKPGQPPKLLIYAATN |
| | LADGVPDRFSGSGSGTDFTLTISSLQAEDVAVYYCHHFWGTPYTFGQGTKVEI |
| | K (SEQ ID NO: 332) |
| Antibody 9F5 | Antibody 9F5 |
| 9F5V2-30 | DVVMTQSPLSLPVTLGQPASISCRSSQSLVHSNGYTYLHWFQQRPGQSPRRLIY |
| | KVSNRFSGVPDRFSGSGSGTDFTLKISRVEAEDVGVYYCSQSTRVPYTFGQGT |
| | KVEIK (SEQ ID NO: 334) |
| 9F5V2-28 | DIVMTQSPLSLPVTPGEPASISCRSSQSLVHSNGYTYLHWYLQKPGQSPQLLIY |
| | KVSNRFSGVPDRFSGSGSGTDFTLKISRVEAEDVGVYYCSQSTRVPYTFGQGT |
| | KVEIK (SEQ ID NO: 335) |
| 9F5V4-1 | DIVMTQSPDSLAVSLGERATINCRSSQSLVHSNGYTYLHWYQQKPGQPPKLLI |
| | YKVSNRFSGVPDRFSGSGSGTDFTLTISSLQAEDVAVYYCSQSTRVPYTFGQGT |
| | KVEIK (SEQ ID NO: 336) |
| 9F5V3-11 | EIVLTQSPATLSLSPGERATLSCRSSQSLVHSNGYTYLHWYQQKPGQAPRLLIY |
| | KVSNRFSGIPARFSGSGSGTDFTLTISSLEPEDFAVYYCSQSTRVPYTFGQGTKV |
| | EIK (SEQ ID NO: 337) |
| 9F5V1-33 | DIQMTQSPSSLSASVGDRVTITCRSSQSLVHSNGYTYLHWYQQKPGKAPKLLI |
| | YKVSNRFSGVPSRFSGSGSGTDFTFTISSLQPEDIATYYCSQSTRVPYTFGQGTK |
| | VEIK (SEQ ID NO: 338) |
| 9F5V3-15 | EIVMTQSPATLSVSPGERATLSCRSSQSLVHSNGYTYLHWYQQKPGQAPRLLI |
| | YKVSNRFSGIPARFSGSGSGTEFTLTISSLQSEDFAVYYCSQSTRVPYTFGQGTK |
| | VEIK (SEQ ID NO: 339) |
| 9F5V1-5 | DIQMTQSPSTLSASVGDRVTITCRSSQSLVHSNGYTYLHWYQQKPGKAPKLLI |
| | YKVSNRFSGVPSRFSGSGSGTEFTLTISSLQPDDFATYYCSQSTRVPYTFGQGT |
| | KVEIK (SEQ ID NO: 340) |
| 9F5V1-39 | DIQMTQSPSSLSASVGDRVTITCRSSQSLVHSNGYTYLHWYQQKPGKAPKLLI |
| | YKVSNRFSGVPSRFSGSGSGTDFTLTISSLQPEDFATYYCSQSTRVPYTFGQGT |
| | KVEIK (SEQ ID NO: 341) |
| 9F5V1-9 | DIQLTQSPSFLSASVGDRVTITCRSSQSLVHSNGYTYLHWYQQKPGKAPKLLIY |
| | KVSNRFSGVPSRFSGSGSGTEFTLTISSLQPEDFATYYCSQSTRVPYTFGQGTKV |
| | EIK (SEQ ID NO: 342) |
| 9F5V3-20 | EIVLTQSPGTLSLSPGERATLSCRSSQSLVHSNGYTYLHWYQQKPGQAPRLLIY |
| | KVSNRFSGIPDRFSGSGSGTDFTLTISRLEPEDFAVYYCSQSTRVPYTFGQGTKV |
| | EIK (SEQ ID NO: 343) |
| 9F5-L1 | DIVMTQTPLSLSVTPGQPASISCRSSQSLVHSNGYTYLHWYLQKPGQSPQLLIY |
| | KVSNRFSGVPDRFSGSGSGTDFTLKISRVEAEDVGVYYCSQSTRVPYTFGQGT |
| | KLEIK (SEQ ID NO: 843) |
| 9F5-L2 | DVVMTQTPLSLSVTPGQPASISCRSSQSLVHSNGYTYLHWYLQKPGQSPQLLI |
| | YKVSNRFSGVPDRFSGSGSGTDFTLKISRVEAEDVGVYYCSQSTRVPYTFGQG |
| | TKLEIK (SEQ ID NO: 844) |
| Antibody 9G1 | Antibody 9G1 |
| 9G1V2-30 | DVVMTQSPLSLPVTLGQPASISCRFSQSLVHSNGNTYLHWFQQRPGQSPRRLIY |
| | KVSNRFSGVPDRFSGSGSGTDFTLKISRVEAEDVGVYYCSQSTRVPPTFGQGT |
| | KVEIK (SEQ ID NO: 345) |
| 9G1V2-28 | DIVMTQSPLSLPVTPGEPASISCRFSQSLVHSNGNTYLHWYLQKPGQSPQLLIY |
| | KVSNRFSGVPDRFSGSGSGTDFTLKISRVEAEDVGVYYCSQSTRVPPTFGQGT |
| | KVEIK (SEQ ID NO: 346) |
| 9G1V4-1 | DIVMTQSPDSLAVSLGERATINCRFSQSLVHSNGNTYLHWYQQKPGQPPKLLI |
| | YKVSNRFSGVPDRFSGSGSGTDFTLTISSLQAEDVAVYYCSQSTRVPPTFGQGT |
| | KVEIK (SEQ ID NO: 347) |
| 9G1V3-11 | EIVLTQSPATLSLSPGERATLSCRFSQSLVHSNGNTYLHWYQQKPGQAPRLLIY |
| | KVSNRFSGIPARFSGSGSGTDFTLTISSLEPEDFAVYYCSQSTRVPPTFGQGTKV |
| | EIK (SEQ ID NO: 348) |
| 9G1V3-15 | EIVMTQSPATLSVSPGERATLSCRFSQSLVHSNGNTYLHWYQQKPGQAPRLLI |
| | YKVSNRFSGIPARFSGSGSGTEFTLTISSLQSEDFAVYYCSQSTRVPPTFGQGTK |
| | VEIK (SEQ ID NO: 349) |
| 9G1V1-9 | DIQLTQSPSFLSASVGDRVTITCRFSQSLVHSNGNTYLHWYQQKPGKAPKLLIY |
| | KVSNRFSGVPSRFSGSGSGTEFTLTISSLQPEDFATYYCSQSTRVPPTFGQGTKV |
| | EIK (SEQ ID NO: 350) |
| 9G1V1-5 | DIQMTQSPSTLSASVGDRVTITCRFSQSLVHSNGNTYLHWYQQKPGKAPKLLI |
| | YKVSNRFSGVPSRFSGSGSGTEFTLTISSLQPDDFATYYCSQSTRVPPTFGQGTK |
| | VEIK (SEQ ID NO: 351) |
| 9G1V1-39 | DIQMTQSPSSLSASVGDRVTITCRFSQSLVHSNGNTYLHWYQQKPGKAPKLLI |
| | YKVSNRFSGVPSRFSGSGSGTDFTLTISSLQPEDFATYYCSQSTRVPPTFGQGTK |
| | VEIK (SEQ ID NO: 352) |
| 9G1V1-33 | DIQMTQSPSSLSASVGDRVTITCRFSQSLVHSNGNTYLHWYQQKPGKAPKLLI |
| | YKVSNRFSGVPSRFSGSGSGTDFTFTISSLQPEDIATYYCSQSTRVPPTFGQGTK |
| | VEIK (SEQ ID NO: 353) |
| 9G1V3-20 | EIVLTQSPGTLSLSPGERATLSCRFSQSLVHSNGNTYLHWYQQKPGQAPRLLIY |
| | KVSNRFSGIPDRFSGSGSGTDFTLTISRLEPEDFAVYYCSQSTRVPPTFGQGTKV |
| | EIK (SEQ ID NO: 354) |
| Antibody 9G3 | Antibody 9G3 |
| 9G3V1-33 | DIQMTQSPSSLSASVGDRVTITCKASSNVNYMSWYQQKPGKAPKLLIYFTSNL |
| | PSGVPSRFSGSGSGTDFTFTISSLQPEDIATYYCSGEVTQFTFGQGTKVEIK (SEQ |
| | ID NO: 356) |
| 9G3V1-9 | DIQLTQSPSFLSASVGDRVTITCKASSNVNYMSWYQQKPGKAPKLLIYFTSNLP |
| | SGVPSRFSGSGSGTEFTLTISSLQPEDFATYYCSGEVTQFTFGQGTKVEIK (SEQ |
| | ID NO: 357) |
| 9G3V1-39 | DIQMTQSPSSLSASVGDRVTITCKASSNVNYMSWYQQKPGKAPKLLIYFTSNL |
| | PSGVPSRFSGSGSGTDFTLTISSLQPEDFATYYCSGEVTQFTFGQGTKVEIK |
| | (SEQ ID NO: 358) |
| 9G3V3-11 | EIVLTQSPATLSLSPGERATLSCKASSNVNYMSWYQQKPGQAPRLLIYFTSNLP |
| | SGIPARFSGSGSGTDFTLTISSLEPEDFAVYYCSGEVTQFTFGQGTKVEIK (SEQ |
| | ID NO: 821) |
| 9G3V1-5 | DIQMTQSPSTLSASVGDRVTITCKASSNVNYMSWYQQKPGKAPKLLIYFTSNL |
| | PSGVPSRFSGSGSGTEFTLTISSLQPDDFATYYCSGEVTQFTFGQGTKVEIK |
| | (SEQ ID NO: 359) |
| 9G3V3-15 | EIVMTQSPATLSVSPGERATLSCKASSNVNYMSWYQQKPGQAPRLLIYFTSNL |
| | PSGIPARFSGSGSGTEFTLTISSLQSEDFAVYYCSGEVTQFTFGQGTKVEIK (SEQ |
| | ID NO: 360) |
| 9G3V3-20 | EIVLTQSPGTLSLSPGERATLSCKASSNVNYMSWYQQKPGQAPRLLIYFTSNLP |
| | SGIPDRFSGSGSGTDFTLTISRLEPEDFAVYYCSGEVTQFTFGQGTKVEIK (SEQ |
| | ID NO: 361) |
| 9G3V2-28 | DIVMTQSPLSLPVTPGEPASISCKASSNVNYMSWYLQKPGQSPQLLIYFTSNLPS |
| | GVPDRFSGSGSGTDFTLKISRVEAEDVGVYYCSGEVTQFTFGQGTKVEIK (SEQ |
| | ID NO: 362) |
| 9G3V2-30 | DVVMTQSPLSLPVTLGQPASISCKASSNVNYMSWFQQRPGQSPRRLIYFTSNLP |
| | SGVPDRFSGSGSGTDFTLKISRVEAEDVGVYYCSGEVTQFTFGQGTKVEIK |
| | (SEQ ID NO: 363) |
| 9G3V4-1 | DIVMTQSPDSLAVSLGERATINCKASSNVNYMSWYQQKPGQPPKLLIYFTSNL |
| | PSGVPDRFSGSGSGTDFTLTISSLQAEDVAVYYCSGEVTQFTFGQGTKVEIK |
| | (SEQ ID NO: 364) |
| Antibody 10A9 | Antibody 10A9 |
| 10A9V2-30 | DVVMTQSPLSLPVTLGQPASISCRSSQTIIHSNGNTYLEWFQQRPGQSPRRLIY |
| | KVSNRFCGVPDRFSGSGSGTDFTLKISRVEAEDVGVYYCFQGSHVPYTFGQGT |
| | KVEIK (SEQ ID NO: 366) |
| 10A9V2-28 | DIVMTQSPLSLPVTPGEPASISCRSSQTIIHSNGNTYLEWYLQKPGQSPQLLIYK |
| | VSNRFCGVPDRFSGSGSGTDFTLKISRVEAEDVGVYYCFQGSHVPYTFGQGTK |
| | VEIK (SEQ ID NO: 367) |
| 10A9V4-1 | DIVMTQSPDSLAVSLGERATINCRSSQTIIHSNGNTYLEWYQQKPGQPPKLLIY |
| | KVSNRFCGVPDRFSGSGSGTDFTLTISSLQAEDVAVYYCFQGSHVPYTFGQGT |
| | KVEIK (SEQ ID NO: 368) |
| 10A9V3-11 | EIVLTQSPATLSLSPGERATLSCRSSQTIIHSNGNTYLEWYQQKPGQAPRLLIYK |
| | VSNRFCGIPARFSGSGSGTDFTLTISSLEPEDFAVYYCFQGSHVPYTFGQGTKV |
| | EIK (SEQ ID NO: 369) |
| 10A9V3-15 | EIVMTQSPATLSVSPGERATLSCRSSQTIIHSNGNTYLEWYQQKPGQAPRLLIY |
| | KVSNRFCGIPARFSGSGSGTEFTLTISSLQSEDFAVYYCFQGSHVPYTFGQGTK |
| | VEIK (SEQ ID NO: 370) |
| 10A9V1-33 | DIQMTQSPSSLSASVGDRVTITCRSSQTIIHSNGNTYLEWYQQKPGKAPKLLIY |
| | KVSNRFCGVPSRFSGSGSGTDFTFTISSLQPEDIATYYCFQGSHVPYTFGQGTK |
| | VEIK (SEQ ID NO: 371) |
| 10A9V3-20 | EIVLTQ SPGTLSLSPGERATLSCRSSQTIIHSNGNTYLEWYQQKPGQAPRLLIYK |
| | VSNRFCGIPDRFSGSGSGTDFTLTISRLEPEDFAVYYCFQGSHVPYTFGQGTKV |
| | EIK (SEQ ID NO: 372) |
| 10A9V1-9 | DIQLTQSPSFLSASVGDRVTITCRSSQTIIHSNGNTYLEWYQQKPGKAPKLLIY |
| | KVSNRFCGVPSRFSGSGSGTEFTLTISSLQPEDFATYYCFQGSHVPYTFGQGTK |
| | VEIK (SEQ ID NO: 373) |
| 10A9V1-5 | DIQMTQSPSTLSASVGDRVTITCRSSQTIIHSNGNTYLEWYQQKPGKAPKLLIY |
| | KVSNRFCGVPSRFSGSGSGTEFTLTISSLQPDDFATYYCFQGSHVPYTFGQGTK |
| | VEIK (SEQ ID NO: 374) |
| 10A9V1-39 | DIQMTQSPSSLSASVGDRVTITCRSSQTIIHSNGNTYLEWYQQKPGKAPKLLIY |
| | KVSNRFCGVPSRFSGSGSGTDFTLTISSLQPEDFATYYCFQGSHVPYTFGQGTK |
| | VEIK (SEQ ID NO: 375) |
| Antibody 11A8 | Antibody 11A8 |
| 11A8V4-1 | DIVMTQSPDSLAVSLGERATINCKSSQSLLNSGNQKKYLTWYQQKPGQPPKLL |
| | IYWASTRESGVPDRFSGSGSGTDFTLTISSLQAEDVAVYYCQNDYGFPLTFGQ |
| | GTKVEIK (SEQ ID NO: 377) |
| 11A8V2-30 | DVVMTQSPLSLPVTLGQPASISCKSSQSLLNSGNQKKYLTWFQQRPGQSPRRLI |
| | YWASTRESGVPDRFSGSGSGTDFTLKISRVEAEDVGVYYCQNDYGFPLTFGQ |
| | GTKVEIK (SEQ ID NO: 378) |
| 11A8V2-28 | DIVMTQSPLSLPVTPGEPASISCKSSQSLLNSGNQKKYLTWYLQKPGQSPQLLI |
| | YWASTRESGVPDRFSGSGSGTDFTLKISRVEAEDVGVYYCQNDYGFPLTFGQ |
| | GTKVEIK (SEQ ID NO: 379) |
| 11A8V1-33 | DIQMTQSPSSLSASVGDRVTITCKSSQSLLNSGNQKKYLTWYQQKPGKAPKLL |
| | IYWASTRESGVPSRFSGSGSGTDFTFTISSLQPEDIATYYCQNDYGFPLTFGQGT |
| | KVEIK (SEQ ID NO: 380) |
| 11A8V3-11 | EIVLTQSPATLSLSPGERATLSCKSSQSLLNSGNQKKYLTWYQQKPGQAPRLLI |
| | YWASTRESGIPARFSGSGSGTDFTLTISSLEPEDFAVYYCQNDYGFPLTFGQGT |
| | KVEIK (SEQ ID NO: 381) |
| 11A8V3-15 | EIVMTQSPATLSVSPGERATLSCKSSQSLLNSGNQKKYLTWYQQKPGQAPRLL |
| | IYWASTRESGIPARFSGSGSGTEFTLTISSLQSEDFAVYYCQNDYGFPLTFGQGT |
| | KVEIK (SEQ ID NO: 382) |
| 11A8V1-5 | DIQMTQSPSTLSASVGDRVTITCKSSQSLLNSGNQKKYLTWYQQKPGKAPKLL |
| | IYWASTRESGVPSRFSGSGSGTEFTLTISSLQPDDFATYYCQNDYGFPLTFGQG |
| | TKVEIK (SEQ ID NO: 383) |
| 11A8V3-20 | EIVLTQSPGTLSLSPGERATLSCKSSQSLLNSGNQKKYLTWYQQKPGQAPRLLI |
| | YWASTRESGIPDRFSGSGSGTDFTLTISRLEPEDFAVYYCQNDYGFPLTFGQGT |
| | KVEIK (SEQ ID NO: 384) |
| 11A8V1-9 | DIQLTQSPSFLSASVGDRVTITCKSSQSLLNSGNQKKYLTWYQQKPGKAPKLLI |
| | YWASTRESGVPSRFSGSGSGTEFTLTISSLQPEDFATYYCQNDYGFPLTFGQGT |
| | KVEIK (SEQ ID NO: 385) |
| 11A8V1-39 | DIQMTQSPSSLSASVGDRVTITCKSSQSLLNSGNQKKYLTWYQQKPGKAPKLL |
| | IYWASTRESGVPSRFSGSGSGTDFTLTISSLQPEDFATYYCQNDYGFPLTFGQG |
| | TKVEIK (SEQ ID NO: 386) |
| Antibody 12D9 | Antibody 12D9 |
| 12D9V4-1 | DIVMTQSPDSLAVSLGERATINCKSSQSLLYSGNQKNFLAWYQQKPGQPPKLL |
| | IYWASTRESGVPDRFSGSGSGTDFTLTISSLQAEDVAVYYCQQYYSYPFTFGQ |
| | GTKVEIK (SEQ ID NO: 388) |
| 12D9V2-28 | DIVMTQSPLSLPVTPGEPASISCKSSQSLLYSGNQKNFLAWYLQKPGQSPQLLI |
| | YWASTRESGVPDRFSGSGSGTDFTLKISRVEAEDVGVYYCQQYYSYPFTFGQG |
| | TKVEIK (SEQ ID NO: 389) |
| 12D9V2-30 | DVVMTQSPLSLPVTLGQPASISCKSSQSLLYSGNQKNFLAWFQQRPGQSPRRLI |
| | YWASTRESGVPDRFSGSGSGTDFTLKISRVEAEDVGVYYCQQYYSYPFTFGQG |
| | TKVEIK (SEQ ID NO: 390) |
| 12D9V1-9 | DIQLTQSPSFLSASVGDRVTITCKSSQSLLYSGNQKNFLAWYQQKPGKAPKLLI |
| | YWASTRESGVPSRFSGSGSGTEFTLTISSLQPEDFATYYCQQYYSYPFTFGQGT |
| | KVEIK (SEQ ID NO: 391) |
| 12D9V1-5 | DIQMTQSPSTLSASVGDRVTITCKSSQSLLYSGNQKNFLAWYQQKPGKAPKLL |
| | IYWASTRESGVPSRFSGSGSGTEFTLTISSLQPDDFATYYCQQYYSYPFTFGQG |
| | TKVEIK (SEQ ID NO: 392) |
| 12D9V3-15 | EIVMTQSPATLSVSPGERATLSCKSSQSLLYSGNQKNFLAWYQQKPGQAPRLL |
| | IYWASTRESGIPARFSGSGSGTEFTLTISSLQSEDFAVYYCQQYYSYPFTFGQGT |
| | KVEIK (SEQ ID NO: 393) |
| 12D9V1-33 | DIQMTQSPSSLSASVGDRVTITCKSSQSLLYSGNQKNFLAWYQQKPGKAPKLL |
| | IYWASTRESGVPSRFSGSGSGTDFTFTISSLQPEDIATYYCQQYYSYPFTFGQGT |
| | KVEIK (SEQ ID NO: 394) |
| 12D9V3-11 | EIVLTQSPATLSLSPGERATLSCKSSQSLLYSGNQKNFLAWYQQKPGQAPRLLI |
| | YWASTRESGIPARFSGSGSGTDFTLTISSLEPEDFAVYYCQQYYSYPFTFGQGT |
| | KVEIK (SEQ ID NO: 395) |
| 12D9V1-39 | DIQMTQSPSSLSASVGDRVTITCKSSQSLLYSGNQKNFLAWYQQKPGKAPKLL |
| | IYWASTRESGVPSRFSGSGSGTDFTLTISSLQPEDFATYYCQQYYSYPFTFGQG |
| | TKVEIK (SEQ ID NO: 396) |
| 12D9V3-20 | EIVLTQSPGTLSLSPGERATLSCKSSQSLLYSGNQKNFLAWYQQKPGQAPRLLI |
| | YWASTRESGIPDRFSGSGSGTDFTLTISRLEPEDFAVYYCQQYYSYPFTFGQGT |
| | KVEIK (SEQ ID NO: 397) |
| Antibody 12F9 | Antibody 12F9 |
| 12F9V4-1 | DIVMTQSPDSLAVSLGERATINCKSSQSLLYSSDQKNYLAWYQQKPGQPPKLL |
| | IYWASTRESGVPDRFSGSGSGTDFTLTISSLQAEDVAVYYCQQYYSYPLTFGQ |
| | GTKVEIK (SEQ ID NO: 300) |
| 12F9V2-28 | DIVMTQSPLSLPVTPGEPASISCKSSQSLLYSSDQKNYLAWYLQKPGQSPQLLI |
| | YWASTRESGVPDRFSGSGSGTDFTLKISRVEAEDVGVYYCQQYYSYPLTFGQ |
| | GTKVEIK (SEQ ID NO: 301) |
| 12F9V2-30 | DVVMTQSPLSLPVTLGQPASISCKSSQSLLYSSDQKNYLAWFQQRPGQSPRRLI |
| | YWASTRESGVPDRFSGSGSGTDFTLKISRVEAEDVGVYYCQQYYSYPLTFGQ |
| | GTKVEIK (SEQ ID NO: 302) |
| 12F9V3-15 | EIVMTQSPATLSVSPGERATLSCKSSQSLLYSSDQKNYLAWYQQKPGQAPRLLI |
| | YWASTRESGIPARFSGSGSGTEFTLTISSLQSEDFAVYYCQQYYSYPLTFGQGT |
| | KVEIK (SEQ ID NO: 305) |
| 12F9V1-5 | DIQMTQSPSTLSASVGDRVTITCKSSQSLLYSSDQKNYLAWYQQKPGKAPKLL |
| | IYWASTRESGVPSRFSGSGSGTEFTLTISSLQPDDFATYYCQQYYSYPLTFGQG |
| | TKVEIK (SEQ ID NO: 303) |
| 12F9V1-9 | DIQLTQSPSFLSASVGDRVTITCKSSQSLLYSSDQKNYLAWYQQKPGKAPKLLI |
| | YWASTRESGVPSRFSGSGSGTEFTLTISSLQPEDFATYYCQQYYSYPLTFGQGT |
| | KVEIK (SEQ ID NO: 304) |
| 12F9V1-33 | DIQMTQSPSSLSASVGDRVTITCKSSQSLLYSSDQKNYLAWYQQKPGKAPKLLI |
| | YWASTRESGVPSRFSGSGSGTDFTFTISSLQPEDIATYYCQQYYSYPLTFGQGT |
| | KVEIK (SEQ ID NO: 308) |
| 12F9V3-11 | EIVLTQSPATLSLSPGERATLSCKSSQSLLYSSDQKNYLAWYQQKPGQAPRLLI |
| | YWASTRESGIPARFSGSGSGTDFTLTISSLEPEDFAVYYCQQYYSYPLTFGQGT |
| | KVEIK (SEQ ID NO: 307) |
| 12F9V1-39 | DIQMTQSPSSLSASVGDRVTITCKSSQSLLYSSDQKNYLAWYQQKPGKAPKLLI |
| | YWASTRESGVPSRFSGSGSGTDFTLTISSLQPEDFATYYCQQYYSYPLTFGQGT |
| | KVEIK (SEQ ID NO: 306) |
| 12F9V3-20 | EIVLTQSPGTLSLSPGERATLSCKSSQSLLYSSDQKNYLAWYQQKPGQAPRLLI |
| | YWASTRESGIPDRFSGSGSGTDFTLTISRLEPEDFAVYYCQQYYSYPLTFGQGT |
| | KVEIK (SEQ ID NO: 309) |
| Antibody 10C1 | Antibody 10C1 |
| 10C1V4-1 | DIVMTQSPDSLAVSLGERATINCKSSQSVFYSSNQKNYLAWYQQKPGQPPKLL |
| | IYWASTRESGVPDRFSGSGSGTDFTLTISSLQAEDVAVYYCHQYLSSLTFGQGT |
| | KVEIK (SEQ ID NO: 603) |
| 10C1V2-30 | DVVMTQSPLSLPVTLGQPASISCKSSQSVFYSSNQKNYLAWFQQRPGQSPRRLI |
| | YWASTRESGVPDRFSGSGSGTDFTLKISRVEAEDVGVYYCHQYLSSLTFGQGT |
| | KVEIK (SEQ ID NO: 604) |
| 10C1V2-28 | DIVMTQSPLSLPVTPGEPASISCKSSQSVFYSSNQKNYLAWYLQKPGQSPQLLI |
| | YWASTRESGVPDRFSGSGSGTDFTLKISRVEAEDVGVYYCHQYLSSLTFGQGT |
| | KVEIK (SEQ ID NO: 605) |
| 10C1V1-5 | DIQMTQSPSTLSASVGDRVTITCKSSQSVEYSSNQKNYLAWYQQKPGKAPKLL |
| | IYWASTRESGVPSRFSGSGSGTEFTLTISSLQPDDFATYYCHQYLSSLTFGQGT |
| | KVEIK (SEQ ID NO: 606) |
| 10C1V3-15 | EIVMTQSPATLSVSPGERATLSCKSSQSVEYSSNQKNYLAWYQQKPGQAPRLLI |
| | YWASTRESGIPARFSGSGSGTEFTLTISSLQSEDFAVYYCHQYLSSLTFGQGTK |
| | VEIK (SEQ ID NO: 607) |
| 10C1V1-9 | DIQLTQSPSFLSASVGDRVTITCKSSQSVEYSSNQKNYLAWYQQKPGKAPKLLI |
| | YWASTRESGVPSRFSGSGSGTEFTLTISSLQPEDFATYYCHQYLSSLTFGQGTK |
| | VEIK (SEQ ID NO: 608) |
| 10C1V3-11 | EIVLTQSPATLSLSPGERATLSCKSSQSVEYSSNQKNYLAWYQQKPGQAPRLLI |
| | YWASTRESGIPARFSGSGSGTDFTLTISSLEPEDFAVYYCHQYLSSLTFGQGTK |
| | VEIK (SEQ ID NO: 609) |
| 10C1V1-39 | DIQMTQSPSSLSASVGDRVTITCKSSQSVEYSSNQKNYLAWYQQKPGKAPKLLI |
| | YWASTRESGVPSRFSGSGSGTDFTLTISSLQPEDFATYYCHQYLSSLTFGQGTK |
| | VEIK (SEQ ID NO: 610) |
| 10C1V1-33 | DIQMTQSPSSLSASVGDRVTITCKSSQSVEYSSNQKNYLAWYQQKPGKAPKLLI |
| | YWASTRESGVPSRFSGSGSGTDFTFTISSLQPEDIATYYCHQYLSSLTFGQGTK |
| | VEIK (SEQ ID NO: 611) |
| 10C1V3-20 | EIVLTQSPGTLSLSPGERATLSCKSSQSVEYSSNQKNYLAWYQQKPGQAPRLLI |
| | YWASTRESGIPDRFSGSGSGTDFTLTISRLEPEDFAVYYCHQYLSSLTFGQGTK |
| | VEIK (SEQ ID NO: 612) |
| Antibody 7E9 | Antibody 7E9 |
| 7E9V4-1 | DIVMTQSPDSLAVSLGERATINCKSSQSLLYSSNQKNCLAWYQQKPGQPPKLL |
| | IYWASTRESGVPDRFSGSGSGTDFTLTISSLQAEDVAVYYCQQYYSYPLTFGQ |
| | GTKVEIK (SEQ ID NO: 614) |
| 7E9V2-28 | DIVMTQSPLSLPVTPGEPASISCKSSQSLLYSSNQKNCLAWYLQKPGQSPQLLI |
| | YWASTRESGVPDRFSGSGSGTDFTLKISRVEAEDVGVYYCQQYYSYPLTFGQ |
| | GTKVEIK (SEQ ID NO: 615) |
| 7E9V2-30 | DVVMTQSPLSLPVTLGQPASISCKSSQSLLYSSNQKNCLAWFQQRPGQSPRRLI |
| | YWASTRESGVPDRFSGSGSGTDFTLKISRVEAEDVGVYYCQQYYSYPLTFGQ |
| | GTKVEIK (SEQ ID NO: 616) |
| 7E9V1-9 | DIQLTQSPSFLSASVGDRVTITCKSSQSLLYSSNQKNCLAWYQQKPGKAPKLLI |
| | YWASTRESGVPSRFSGSGSGTEFTLTISSLQPEDFATYYCQQYYSYPLTFGQGT |
| | KVEIK (SEQ ID NO: 617) |
| 7E9V3-15 | EIVMTQSPATLSVSPGERATLSCKSSQSLLYSSNQKNCLAWYQQKPGQAPRLLI |
| | YWASTRESGIPARFSGSGSGTEFTLTISSLQSEDFAVYYCQQYYSYPLTFGQGT |
| | KVEIK (SEQ ID NO: 618) |
| 7E9V1-5 | DIQMTQSPSTLSASVGDRVTITCKSSQSLLYSSNQKNCLAWYQQKPGKAPKLL |
| | IYWASTRESGVPSRFSGSGSGTEFTLTISSLQPDDFATYYCQQYYSYPLTFGQG |
| | TKVEIK (SEQ ID NO: 619) |
| 7E9V1-33 | DIQMTQSPSSLSASVGDRVTITCKSSQSLLYSSNQKNCLAWYQQKPGKAPKLLI |
| | YWASTRESGVPSRFSGSGSGTDFTFTISSLQPEDIATYYCQQYYSYPLTFGQGT |
| | KVEIK (SEQ ID NO: 620) |
| 7E9V1-39 | DIQMTQSPSSLSASVGDRVTITCKSSQSLLYSSNQKNCLAWYQQKPGKAPKLLI |
| | YWASTRESGVPSRFSGSGSGTDFTLTISSLQPEDFATYYCQQYYSYPLTFGQGT |
| | KVEIK (SEQ ID NO: 621) |
| 7E9V3-11 | EIVLTQSPATLSLSPGERATLSCKSSQSLLYSSNQKNCLAWYQQKPGQAPRLLI |
| | YWASTRESGIPARFSGSGSGTDFTLTISSLEPEDFAVYYCQQYYSYPLTFGQGT |
| | KVEIK (SEQ ID NO: 622) |
| 7E9V3-20 | EIVLTQSPGTLSLSPGERATLSCKSSQSLLYSSNQKNCLAWYQQKPGQAPRLLI |
| | YWASTRESGIPDRFSGSGSGTDFTLTISRLEPEDFAVYYCQQYYSYPLTFGQGT |
| | KVEIK (SEQ ID NO: 623) |
| Antibody 8C3 | Antibody 8C3 |
| 8C3V2-30 | DVVMTQSPLSLPVTLGQPASISCRSSQSLVHSNGNTYLHWFQQRPGQSPRRLIY |
| | KVSNRFSGVPDRFSGSGSGTDFTLKISRVEAEDVGVYYCSQSTHVPPTFGQGT |
| | KVEIK (SEQ ID NO: 625) |
| 8C3V2-28 | DIVMTQSPLSLPVTPGEPASISCRSSQSLVHSNGNTYLHWYLQKPGQSPQLLIY |
| | KVSNRFSGVPDRFSGSGSGTDFTLKISRVEAEDVGVYYCSQSTHVPPTFGQGT |
| | KVEIK (SEQ ID NO: 626) |
| 8C3V4-1 | DIVMTQSPDSLAVSLGERATINCRSSQSLVHSNGNTYLHWYQQKPGQPPKLLI |
| | YKVSNRFSGVPDRFSGSGSGTDFTLTISSLQAEDVAVYYCSQSTHVPPTFGQGT |
| | KVEIK (SEQ ID NO: 627) |
| 8C3V3-11 | EIVLTQSPATLSLSPGERATLSCRSSQSLVHSNGNTYLHWYQQKPGQAPRLLIY |
| | KVSNRFSGIPARFSGSGSGTDFTLTISSLEPEDFAVYYCSQSTHVPPTFGQGTKV |
| | EIK (SEQ ID NO: 628) |
| 8C3V1-33 | DIQMTQSPSSLSASVGDRVTITCRSSQSLVHSNGNTYLHWYQQKPGKAPKLLI |
| | YKVSNRFSGVPSRFSGSGSGTDFTFTISSLQPEDIATYYCSQSTHVPPTFGQGTK |
| | VEIK (SEQ ID NO: 629) |
| 8C3V1-5 | DIQMTQSPSTLSASVGDRVTITCRSSQSLVHSNGNTYLHWYQQKPGKAPKLLI |
| | YKVSNRFSGVPSRFSGSGSGTEFTLTISSLQPDDFATYYCSQSTHVPPTFGQGTK |
| | VEIK (SEQ ID NO: 630) |
| 8C3V1-39 | DIQMTQSPSSLSASVGDRVTITCRSSQSLVHSNGNTYLHWYQQKPGKAPKLLI |
| | YKVSNRFSGVPSRFSGSGSGTDFTLTISSLQPEDFATYYCSQSTHVPPTFGQGT |
| | KVEIK (SEQ ID NO: 631) |
| 8C3V1-9 | DIQLTQSPSFLSASVGDRVTITCRSSQSLVHSNGNTYLHWYQQKPGKAPKLLIY |
| | KVSNRFSGVPSRFSGSGSGTEFTLTISSLQPEDFATYYCSQSTHVPPTFGQGTKV |
| | EIK (SEQ ID NO: 632) |
| 8C3V3-15 | EIVMTQSPATLSVSPGERATLSCRSSQSLVHSNGNTYLHWYQQKPGQAPRLLI |
| | YKVSNRFSGIPARFSGSGSGTEFTLTISSLQSEDFAVYYCSQSTHVPPTFGQGTK |
| | VEIK (SEQ ID NO: 633) |
| 8C3V3-20 | EIVLTQSPGTLSLSPGERATLSCRSSQSLVHSNGNTYLHWYQQKPGQAPRLLIY |
| | KVSNRFSGIPDRFSGSGSGTDFTLTISRLEPEDFAVYYCSQSTHVPPTFGQGTKV |
| | EIK (SEQ ID NO: 634) |

TABLE 7B
Humanized heavy chain variable region sequences

| Antibody variant | Humanized sequences |
|---|---|
| Antibody 4D11 | Antibody 4D11 |
| 4D11V4-59 | QVQLQESGPGLVKPSETLSLTCTVSGFTLSSYAMSWIRQPPGKGLEWVASISR |
| | GGSTYYPPSLKSRVTISVDTSKNQFSLKLSSVTAADTAVYYCTRGYGYYRTP |
| | FANWGQGTLVTVSS (SEQ ID NO: 400) |
| 4D11V3-23 | EVQLLESGGGLVQPGGSLRLSCAASGFTLSSYAMSWVRQAPGKGLEWVASIS |
| | RGGSTYYPDSVKGRFTISRDNSKNTLYLQMNSLRAEDTAVYYCTRGYGYYR |
| | TPFANWGQGTLVTVSS (SEQ ID NO: 401) |
| 4D11V3-7 | EVQLVESGGGLVQPGGSLRLSCAASGFTLSSYAMSWVRQAPGKGLEWVASIS |
| | RGGSTYYPDSVKGRFTISRDNAKNSLYLQMNSLRAEDTAVYYCTRGYGYYR |
| | TPFANWGQGTLVTVSS (SEQ ID NO: 402) |
| 4D11V3-48 | EVQLVESGGGLVQPGGSLRLSCAASGFTLSSYAMSWVRQAPGKGLEWVASIS |
| | RGGSTYYPDSVKGRFTISRDNAKNSLYLQMNSLRAEDTAVYYCTRGYGYYR |
| | TPFANWGQGTLVTVSS (SEQ ID NO: 402) |
| 4D11V3-30 | QVQLVESGGGVVQPGRSLRLSCAASGFTLSSYAMSWVRQAPGKGLEWVASI |
| | SRGGSTYYPDSVKGRFTISRDNSKNTLYLQMNSLRAEDTAVYYCTRGYGYY |
| | RTPFANWGQGTLVTVSS (SEQ ID NO: 403) |
| 4D11V1-69 | QVQLVQSGAEVKKPGSSVKVSCKASGFTLSSYAMSWVRQAPGQGLEWVASI |
| | SRGGSTYYPQKFQGRVTITADESTSTAYMELSSLRSEDTAVYYCTRGYGYYR |
| | TPFANWGQGTLVTVSS (SEQ ID NO: 404) |
| 4D11V1-46 | QVQLVQSGAEVKKPGASVKVSCKASGFTLSSYAMSWVRQAPGQGLEWVASI |
| | SRGGSTYYPQKFQGRVTMTRDTSTSTVYMELSSLRSEDTAVYYCTRGYGYY |
| | RTPFANWGQGTLVTVSS (SEQ ID NO: 405) |
| 4D11V5-51 | EVQLVQSGAEVKKPGESLKISCKGSGFTLSSYAMSWVRQMPGKGLEWVASIS |
| | RGGSTYYPPSFQGQVTISADKSISTAYLQWSSLKASDTAMYYCTRGYGYYR |
| | TPFANWGQGTLVTVSS (SEQ ID NO: 406) |
| 4D11V4-39 | QLQLQESGPGLVKPSETLSLTCTVSGFTLSSYAMSWIRQPPGKGLEWVASISR |
| | GGSTYYPPSLKSRVTISVDTSKNQFSLKLSSVTAADTAVYYCTRGYGYYRTP |
| | FANWGQGTLVTVSS (SEQ ID NO: 407) |
| 4D11V4-30-4 | QVQLQESGPGLVKPSQTLSLTCTVSGFTLSSYAMSWIRQPPGKGLEWVASISR |
| | GGSTYYPPSLKSRVTISVDTSKNQFSLKLSSVTAADTAVYYCTRGYGYYRTP |
| | FANWGQGTLVTVSS (SEQ ID NO: 408) |
| Antibody 7C5 | Antibody 7C5 |
| 7C5V4-59 | QVQLQESGPGLVKPSETLSLTCTVSGFTLSSYAMSWIRQPPGKGLEWVASISR |
| | GGSTYYPPSLKSRVTISVDTSKNQFSLKLSSVTAADTAVYYCTRGYGYYRTP |
| | FANWGQGTLVTVSS (SEQ ID NO: 400) |
| 7C5V3-23 | EVQLLESGGGLVQPGGSLRLSCAASGFTLSSYAMSWVRQAPGKGLEWVASIS |
| | RGGSTYYPDSVKGRFTISRDNSKNTLYLQMNSLRAEDTAVYYCTRGYGYYR |
| | TPFANWGQGTLVTVSS (SEQ ID NO: 401) |
| 7C5V3-7 | EVQLVESGGGLVQPGGSLRLSCAASGFTLSSYAMSWVRQAPGKGLEWVASIS |
| | RGGSTYYPDSVKGRFTISRDNAKNSLYLQMNSLRAEDTAVYYCTRGYGYYR |
| | TPFANWGQGTLVTVSS (SEQ ID NO: 402) |
| 7C5V3-48 | EVQLVESGGGLVQPGGSLRLSCAASGFTLSSYAMSWVRQAPGKGLEWVASIS |
| | RGGSTYYPDSVKGRFTISRDNAKNSLYLQMNSLRAEDTAVYYCTRGYGYYR |
| | TPFANWGQGTLVTVSS (SEQ ID NO: 402) |
| 7C5V3-30 | QVQLVESGGGVVQPGRSLRLSCAASGFTLSSYAMSWVRQAPGKGLEWVASI |
| | SRGGSTYYPDSVKGRFTISRDNSKNTLYLQMNSLRAEDTAVYYCTRGYGYY |
| | RTPFANWGQGTLVTVSS (SEQ ID NO: 403) |
| 7C5V1-69 | QVQLVQSGAEVKKPGSSVKVSCKASGFTLSSYAMSWVRQAPGQGLEWVASI |
| | SRGGSTYYPQKFQGRVTITADESTSTAYMELSSLRSEDTAVYYCTRGYGYYR |
| | TPFANWGQGTLVTVSS (SEQ ID NO: 404) |
| 7C5V1-46 | QVQLVQSGAEVKKPGASVKVSCKASGFTLSSYAMSWVRQAPGQGLEWVASI |
| | SRGGSTYYPQKFQGRVTMTRDTSTSTVYMELSSLRSEDTAVYYCTRGYGYY |
| | RTPFANWGQGTLVTVSS (SEQ ID NO: 405) |
| 7C5V5-51 | EVQLVQSGAEVKKPGESLKISCKGSGFTLSSYAMSWVRQMPGKGLEWVASIS |
| | RGGSTYYPPSFQGQVTISADKSISTAYLQWSSLKASDTAMYYCTRGYGYYR |
| | TPFANWGQGTLVTVSS (SEQ ID NO: 406) |
| 7C5V4-39 | QLQLQESGPGLVKPSETLSLTCTVSGFTLSSYAMSWIRQPPGKGLEWVASISR |
| | GGSTYYPPSLKSRVTISVDTSKNQFSLKLSSVTAADTAVYYCTRGYGYYRTP |
| | FANWGQGTLVTVSS (SEQ ID NO: 407) |
| 7C5V4-30-4 | QVQLQESGPGLVKPSQTLSLTCTVSGFTLSSYAMSWIRQPPGKGLEWVASISR |
| | GGSTYYPPSLKSRVTISVDTSKNQFSLKLSSVTAADTAVYYCTRGYGYYRTP |
| | FANWGQGTLVTVSS (SEQ ID NO: 408) |
| Antibody 6G12 | Antibody 6G12 |
| 6G12V1-46 | QVQLVQSGAEVKKPGASVKVSCKASGYTFTEYTMHWVRQAPGQGLEWIGG |
| | INPNNGGTSYSQKFQGRVTMTRDTSTSTVYMELSSLRSEDTAVYYCARGGS |
| | HYYAMDYWGQGTLVTVSS (SEQ ID NO: 410) |
| 6G12V5-51 | EVQLVQSGAEVKKPGESLKISCKGSGYTFTEYTMHWVRQMPGKGLEWIGGI |
| | NPNNGGTSYSPSFQGQVTISADKSISTAYLQWSSLKASDTAMYYCARGGSHY |
| | YAMDYWGQGTLVTVSS (SEQ ID NO: 411) |
| 6G12V1-69 | QVQLVQSGAEVKKPGSSVKVSCKASGYTFTEYTMHWVRQAPGQGLEWIGG |
| | INPNNGGTSYSQKFQGRVTITADESTSTAYMELSSLRSEDTAVYYCARGGSH |
| | YYAMDYWGQGTLVTVSS (SEQ ID NO: 412) |
| 6G12V3-23 | EVQLLESGGGLVQPGGSLRLSCAASGYTFTEYTMHWVRQAPGKGLEWIGGI |
| | NPNNGGTSYSDSVKGRFTISRDNSKNTLYLQMNSLRAEDTAVYYCARGGSH |
| | YYAMDYWGQGTLVTVSS (SEQ ID NO: 413) |
| 6G12V3-30 | QVQLVESGGGVVQPGRSLRLSCAASGYTFTEYTMHWVRQAPGKGLEWIGGI |
| | NPNNGGTSYSDSVKGRFTISRDNSKNTLYLQMNSLRAEDTAVYYCARGGSH |
| | YYAMDYWGQGTLVTVSS (SEQ ID NO: 414) |
| 6G12V3-48 | EVQLVESGGGLVQPGGSLRLSCAASGYTFTEYTMHWVRQAPGKGLEWIGGI |
| | NPNNGGTSYSDSVKGRFTISRDNAKNSLYLQMNSLRAEDTAVYYCARGGSH |
| | YYAMDYWGQGTLVTVSS (SEQ ID NO: 415) |
| 6G12V3-7 | EVQLVESGGGLVQPGGSLRLSCAASGYTFTEYTMHWVRQAPGKGLEWIGGI |
| | NPNNGGTSYSDSVKGRFTISRDNAKNSLYLQMNSLRAEDTAVYYCARGGSH |
| | YYAMDYWGQGTLVTVSS (SEQ ID NO: 415) |
| 6G12V4-59 | QVQLQESGPGLVKPSETLSLTCTVSGYTFTEYTMHWIRQPPGKGLEWIGGIN |
| | PNNGGTSYSPSLKSRVTISVDTSKNQFSLKLSSVTAADTAVYYCARGGSHYY |
| | AMDYWGQGTLVTVSS (SEQ ID NO: 416) |
| 6G12V3-15 | EVQLVESGGGLVKPGGSLRLSCAASGYTFTEYTMHWVRQAPGKGLEWIGGI |
| | NPNNGGTSYSAPVKGRFTISRDDSKNTLYLQMNSLKTEDTAVYYCARGGSH |
| | YYAMDYWGQGTLVTVSS (SEQ ID NO: 417) |
| 6G12V4-39 | QLQLQESGPGLVKPSETLSLTCTVSGYTFTEYTMHWIRQPPGKGLEWIGGIN |
| | PNNGGTSYSPSLKSRVTISVDTSKNQFSLKLSSVTAADTAVYYCARGGSHYY |
| | AMDYWGQGTLVTVSS (SEQ ID NO: 418) |
| Antibody 8E10 | Antibody 8E10 |
| 8E10V1-46 | QVQLVQSGAEVKKPGASVKVSCKASGYTFTDYEMHWVRQAPGQGLEWIGV |
| | IDPETGGTAYNQKFQGRVTMTRDTSTSTVYMELSSLRSEDTAVYYCTSPDYY |
| | GSSYPLYYAMDYWGQGTLVTVSS (SEQ ID NO: 420) |
| 8E10V5-51 | EVQLVQSGAEVKKPGESLKISCKGSGYTFTDYEMHWVRQMPGKGLEWIGVI |
| | DPETGGTAYNPSFQGQVTISADKSISTAYLQWSSLKASDTAMYYCTSPDYYG |
| | SSYPLYYAMDYWGQGTLVTVSS (SEQ ID NO: 421) |
| 8E10V3-30 | QVQLVESGGGVVQPGRSLRLSCAASGYTFTDYEMHWVRQAPGKGLEWIGVI |
| | DPETGGTAYNDSVKGRFTISRDNSKNTLYLQMNSLRAEDTAVYYCTSPDYY |
| | GSSYPLYYAMDYWGQGTLVTVSS (SEQ ID NO: 422) |
| 8E10V3-23 | EVQLLESGGGLVQPGGSLRLSCAASGYTFTDYEMHWVRQAPGKGLEWIGVI |
| | DPETGGTAYNDSVKGRFTISRDNSKNTLYLQMNSLRAEDTAVYYCTSPDYY |
| | GSSYPLYYAMDYWGQGTLVTVSS (SEQ ID NO: 423) |
| 8E10V1-69 | QVQLVQSGAEVKKPGSSVKVSCKASGYTFTDYEMHWVRQAPGQGLEWIGV |
| | IDPETGGTAYNQKFQGRVTITADESTSTAYMELSSLRSEDTAVYYCTSPDYY |
| | GSSYPLYYAMDYWGQGTLVTVSS (SEQ ID NO: 424) |
| 8E10V3-48 | EVQLVESGGGLVQPGGSLRLSCAASGYTFTDYEMHWVRQAPGKGLEWIGVI |
| | DPETGGTAYNDSVKGRFTISRDNAKNSLYLQMNSLRAEDTAVYYCTSPDYY |
| | GSSYPLYYAMDYWGQGTLVTVSS (SEQ ID NO: 425) |
| 8E10V3-7 | EVQLVESGGGLVQPGGSLRLSCAASGYTFTDYEMHWVRQAPGKGLEWIGVI |
| | DPETGGTAYNDSVKGRFTISRDNAKNSLYLQMNSLRAEDTAVYYCTSPDYY |
| | GSSYPLYYAMDYWGQGTLVTVSS (SEQ ID NO: 425) |
| 8E10V4-59 | QVQLQESGPGLVKPSETLSLTCTVSGYTFTDYEMHWIRQPPGKGLEWIGVID |
| | PETGGTAYNPSLKSRVTISVDTSKNQFSLKLSSVTAADTAVYYCTSPDYYGSS |
| | YPLYYAMDYWGQGTLVTVSS (SEQ ID NO: 426) |
| 8E10V3-15 | EVQLVESGGGLVKPGGSLRLSCAASGYTFTDYEMHWVRQAPGKGLEWIGVI |
| | DPETGGTAYNAPVKGRFTISRDDSKNTLYLQMNSLKTEDTAVYYCTSPDYY |
| | GSSYPLYYAMDYWGQGTLVTVSS (SEQ ID NO: 427) |
| 8E10V4-39 | QLQLQESGPGLVKPSETLSLTCTVSGYTFTDYEMHWIRQPPGKGLEWIGVID |
| | PETGGTAYNPSLKSRVTISVDTSKNQFSLKLSSVTAADTAVYYCTSPDYYGSS |
| | YPLYYAMDYWGQGTLVTVSS (SEQ ID NO: 428) |
| Antibody 7E5 | Antibody 7E5 |
| 7E5V3-15 | EVQLVESGGGLVKPGGSLRLSCAASGFTFSDAWMGWVRQAPGKGLEWVAE |
| | IRDKVKNHATYYAAPVKGRFTISRDDSKNTLYLQMNSLKTEDTAVYYCRLG |
| | VFDYWGQGTLVTVSS (SEQ ID NO: 430) |
| 7E5V3-7 | EVQLVESGGGLVQPGGSLRLSCAASGFTFSDAWMGWVRQAPGKGLEWVAE |
| | IRDKVKNHATYYADSVKGRFTISRDNAKNSLYLQMNSLRAEDTAVYYCRLG |
| | VFDYWGQGTLVTVSS (SEQ ID NO: 431) |
| 7E5V3-23 | EVQLLESGGGLVQPGGSLRLSCAASGFTFSDAWMGWVRQAPGKGLEWVAE |
| | IRDKVKNHATYYADSVKGRFTISRDNSKNTLYLQMNSLRAEDTAVYYCRLG |
| | VFDYWGQGTLVTVSS (SEQ ID NO: 432) |
| 7E5V3-48 | EVQLVESGGGLVQPGGSLRLSCAASGFTFSDAWMGWVRQAPGKGLEWVAE |
| | IRDKVKNHATYYADSVKGRFTISRDNAKNSLYLQMNSLRAEDTAVYYCRLG |
| | VFDYWGQGTLVTVSS (SEQ ID NO: 431) |
| 7E5V3-30 | QVQLVESGGGVVQPGRSLRLSCAASGFTFSDAWMGWVRQAPGKGLEWVAE |
| | IRDKVKNHATYYADSVKGRFTISRDNSKNTLYLQMNSLRAEDTAVYYCRLG |
| | VFDYWGQGTLVTVSS (SEQ ID NO: 433) |
| 7E5V1-69 | QVQLVQSGAEVKKPGSSVKVSCKASGFTFSDAWMGWVRQAPGQGLEWVA |
| | EIRDKVKNHATYYAQKFQGRVTITADESTSTAYMELSSLRSEDTAVYYCRLG |
| | VFDYWGQGTLVTVSS (SEQ ID NO: 434) |
| 7E5V1-46 | QVQLVQSGAEVKKPGASVKVSCKASGFTFSDAWMGWVRQAPGQGLEWVA |
| | EIRDKVKNHATYYAQKFQGRVTMTRDTSTSTVYMELSSLRSEDTAVYYCRL |
| | GVFDYWGQGTLVTVSS (SEQ ID NO: 435) |
| 7E5V5-51 | EVQLVQSGAEVKKPGESLKISCKGSGFTFSDAWMGWVRQMPGKGLEWVAE |
| | IRDKVKNHATYYAPSFQGQVTISADKSISTAYLQWSSLKASDTAMYYCRLG |
| | VFDYWGQGTLVTVSS (SEQ ID NO: 436) |
| 7E5V4-59 | QVQLQESGPGLVKPSETLSLTCTVSGFTFSDAWMGWIRQPPGKGLEWVAEIR |
| | DKVKNHATYYAPSLKSRVTISVDTSKNQFSLKLSSVTAADTAVYYCRLGVFD |
| | YWGQGTLVTVSS (SEQ ID NO: 437) |
| 7E5V4-39 | QLQLQESGPGLVKPSETLSLTCTVSGFTFSDAWMGWIRQPPGKGLEWVAEIR |
| | DKVKNHATYYAPSLKSRVTISVDTSKNQFSLKLSSVTAADTAVYYCRLGVFD |
| | YWGQGTLVTVSS (SEQ ID NO: 438) |
| Antibody 7F8 | Antibody 7F8 |
| 7F8V3-15 | EVQLVESGGGLVKPGGSLRLSCAASGFSFNTYAMNWVRQAPGKGLEWIARI |
| | RSKSNNYATYYAAPVKGRFTISRDDSKNTLYLQMNSLKTEDTAVYYCVRHG |
| | DGNLWYIDVWGQGTLVTVSS (SEQ ID NO: 440) |
| 7F8V3-48 | EVQLVESGGGLVQPGGSLRLSCAASGFSFNTYAMNWVRQAPGKGLEWIARI |
| | RSKSNNYATYYADSVKGRFTISRDNAKNSLYLQMNSLRAEDTAVYYCVRHG |
| | DGNLWYIDVWGQGTLVTVSS (SEQ ID NO: 441) |
| 7F8V3-23 | EVQLLESGGGLVQPGGSLRLSCAASGFSFNTYAMNWVRQAPGKGLEWIARI |
| | RSKSNNYATYYADSVKGRFTISRDNSKNTLYLQMNSLRAEDTAVYYCVRHG |
| | DGNLWYIDVWGQGTLVTVSS (SEQ ID NO: 442) |
| 7F8V3-7 | EVQLVESGGGLVQPGGSLRLSCAASGFSFNTYAMNWVRQAPGKGLEWIARI |
| | RSKSNNYATYYADSVKGRFTISRDNAKNSLYLQMNSLRAEDTAVYYCVRHG |
| | DGNLWYIDVWGQGTLVTVSS (SEQ ID NO: 441) |
| 7F8V3-30 | QVQLVESGGGVVQPGRSLRLSCAASGFSFNTYAMNWVRQAPGKGLEWIARI |
| | RSKSNNYATYYADSVKGRFTISRDNSKNTLYLQMNSLRAEDTAVYYCVRHG |
| | DGNLWYIDVWGQGTLVTVSS (SEQ ID NO: 443) |
| 7F8V1-69 | QVQLVQSGAEVKKPGSSVKVSCKASGFSFNTYAMNWVRQAPGQGLEWIARI |
| | RSKSNNYATYYAQKFQGRVTITADESTSTAYMELSSLRSEDTAVYYCVRHG |
| | DGNLWYIDVWGQGTLVTVSS (SEQ ID NO: 444) |
| 7F8V5-51 | EVQLVQSGAEVKKPGESLKISCKGSGFSFNTYAMNWVRQMPGKGLEWIARI |
| | RSKSNNYATYYAPSFQGQVTISADKSISTAYLQWSSLKASDTAMYYCVRHG |
| | DGNLWYIDVWGQGTLVTVSS (SEQ ID NO: 445) |
| 7F8V1-46 | QVQLVQSGAEVKKPGASVKVSCKASGFSFNTYAMNWVRQAPGQGLEWIAR |
| | IRSKSNNYATYYAQKFQGRVTMTRDTSTSTVYMELSSLRSEDTAVYYCVRH |
| | GDGNLWYIDVWGQGTLVTVSS (SEQ ID NO: 446) |
| 7F8V4-59 | QVQLQESGPGLVKPSETLSLTCTVSGFSFNTYAMNWIRQPPGKGLEWIARIRS |
| | KSNNYATYYAPSLKSRVTISVDTSKNQFSLKLSSVTAADTAVYYCVRHGDGN |
| | LWYIDVWGQGTLVTVSS (SEQ ID NO: 447) |
| 7F8V4-30-4 | QVQLQESGPGLVKPSQTLSLTCTVSGFSFNTYAMNWIRQPPGKGLEWIARIRS |
| | KSNNYATYYAPSLKSRVTISVDTSKNQFSLKLSSVTAADTAVYYCVRHGDGN |
| | LWYIDVWGQGTLVTVSS (SEQ ID NO: 448) |
| Antibody 8F8 | Antibody 8F8 |
| 8F8V1-46 | QVQLVQSGAEVKKPGASVKVSCKASGYTVSRYWMHWVRQAPGQGLEWIG |
| | RIDPNSGGTKYNQKFQGRVTMTRDTSTSTVYMELSSLRSEDTAVYYCVLTG |
| | TDFDYWGQGTLVTVSS (SEQ ID NO: 450) |
| 8F8V3-23 | EVQLLESGGGLVQPGGSLRLSCAASGYTVSRYWMHWVRQAPGKGLEWIGR |
| | IDPNSGGTKYNDSVKGRFTISRDNSKNTLYLQMNSLRAEDTAVYYCVLTGT |
| | DFDYWGQGTLVTVSS (SEQ ID NO: 451) |
| 8F8V1-69 | QVQLVQSGAEVKKPGSSVKVSCKASGYTVSRYWMHWVRQAPGQGLEWIG |
| | RIDPNSGGTKYNQKFQGRVTITADESTSTAYMELSSLRSEDTAVYYCVLTGT |
| | DFDYWGQGTLVTVSS (SEQ ID NO: 452) |
| 8F8V5-51 | EVQLVQSGAEVKKPGESLKISCKGSGYTVSRYWMHWVRQMPGKGLEWIGR |
| | IDPNSGGTKYNPSFQGQVTISADKSISTAYLQWSSLKASDTAMYYCVLTGTD |
| | FDYWGQGTLVTVSS (SEQ ID NO: 453) |
| 8F8V3-48 | EVQLVESGGGLVQPGGSLRLSCAASGYTVSRYWMHWVRQAPGKGLEWIGR |
| | IDPNSGGTKYNDSVKGRFTISRDNAKNSLYLQMNSLRAEDTAVYYCVLTGT |
| | DFDYWGQGTLVTVSS (SEQ ID NO: 454) |
| 8F8V3-30 | QVQLVESGGGVVQPGRSLRLSCAASGYTVSRYWMHWVRQAPGKGLEWIGR |
| | IDPNSGGTKYNDSVKGRFTISRDNSKNTLYLQMNSLRAEDTAVYYCVLTGT |
| | DFDYWGQGTLVTVSS (SEQ ID NO: 455) |
| 8F8V3-7 | EVQLVESGGGLVQPGGSLRLSCAASGYTVSRYWMHWVRQAPGKGLEWIGR |
| | IDPNSGGTKYNDSVKGRFTISRDNAKNSLYLQMNSLRAEDTAVYYCVLTGT |
| | DFDYWGQGTLVTVSS (SEQ ID NO: 454) |
| 8F8V4-59 | QVQLQESGPGLVKPSETLSLTCTVSGYTVSRYWMHWIRQPPGKGLEWIGRID |
| | PNSGGTKYNPSLKSRVTISVDTSKNQFSLKLSSVTAADTAVYYCVLTGTDFD |
| | YWGQGTLVTVSS (SEQ ID NO: 456) |
| 8F8V3-15 | EVQLVESGGGLVKPGGSLRLSCAASGYTVSRYWMHWVRQAPGKGLEWIGR |
| | IDPNSGGTKYNAPVKGRFTISRDDSKNTLYLQMNSLKTEDTAVYYCVLTGT |
| | DFDYWGQGTLVTVSS (SEQ ID NO: 457) |
| 8F8V4-30-4 | QVQLQESGPGLVKPSQTLSLTCTVSGYTVSRYWMHWIRQPPGKGLEWIGRID |
| | PNSGGTKYNPSLKSRVTISVDTSKNQFSLKLSSVTAADTAVYYCVLTGTDFD |
| | YWGQGTLVTVSS (SEQ ID NO: 458) |
| Antibody 1H7 | Antibody 1H7 |
| 1H7V3-15 | EVQLVESGGGLVKPGGSLRLSCAASGFSFNTYAMNWVRQAPGKGLEWIARI |
| | RSKSNNYATYYAAPVKGRFTISRDDSKNTLYLQMNSLKTEDTAVYYCVRHG |
| | DGNLWYIDVWGQGTLVTVSS (SEQ ID NO: 440) |
| 1H7V3-23 | EVQLLESGGGLVQPGGSLRLSCAASGFSFNTYAMNWVRQAPGKGLEWIARI |
| | RSKSNNYATYYADSVKGRFTISRDNSKNTLYLQMNSLRAEDTAVYYCVRHG |
| | DGNLWYIDVWGQGTLVTVSS (SEQ ID NO: 442) |
| 1H7V3-48 | EVQLVESGGGLVQPGGSLRLSCAASGFSFNTYAMNWVRQAPGKGLEWIARI |
| | RSKSNNYATYYADSVKGRFTISRDNAKNSLYLQMNSLRAEDTAVYYCVRHG |
| | DGNLWYIDVWGQGTLVTVSS (SEQ ID NO: 441) |
| 1H7V3-7 | EVQLVESGGGLVQPGGSLRLSCAASGFSFNTYAMNWVRQAPGKGLEWIARI |
| | RSKSNNYATYYADSVKGRFTISRDNAKNSLYLQMNSLRAEDTAVYYCVRHG |
| | DGNLWYIDVWGQGTLVTVSS (SEQ ID NO: 441) |
| 1H7V3-30 | QVQLVESGGGVVQPGRSLRLSCAASGFSFNTYAMNWVRQAPGKGLEWIARI |
| | RSKSNNYATYYADSVKGRFTISRDNSKNTLYLQMNSLRAEDTAVYYCVRHG |
| | DGNLWYIDVWGQGTLVTVSS (SEQ ID NO: 443) |
| 1H7V5-51 | EVQLVQSGAEVKKPGESLKISCKGSGFSFNTYAMNWVRQMPGKGLEWIARI |
| | RSKSNNYATYYAPSFQGQVTISADKSISTAYLQWSSLKASDTAMYYCVRHG |
| | DGNLWYIDVWGQGTLVTVSS (SEQ ID NO: 445) |
| 1H7V1-69 | QVQLVQSGAEVKKPGSSVKVSCKASGFSFNTYAMNWVRQAPGQGLEWIARI |
| | RSKSNNYATYYAQKFQGRVTITADESTSTAYMELSSLRSEDTAVYYCVRHG |
| | DGNLWYIDVWGQGTLVTVSS (SEQ ID NO: 444) |
| 1H7V1-46 | QVQLVQSGAEVKKPGASVKVSCKASGFSFNTYAMNWVRQAPGQGLEWIAR |
| | IRSKSNNYATYYAQKFQGRVTMTRDTSTSTVYMELSSLRSEDTAVYYCVRH |
| | GDGNLWYIDVWGQGTLVTVSS (SEQ ID NO: 446) |
| 1H7V4-59 | QVQLQESGPGLVKPSETLSLTCTVSGFSFNTYAMNWIRQPPGKGLEWIARIRS |
| | KSNNYATYYAPSLKSRVTISVDTSKNQFSLKLSSVTAADTAVYYCVRHGDGN |
| | LWYIDVWGQGTLVTVSS (SEQ ID NO: 447) |
| 1H7V4-30-4 | QVQLQESGPGLVKPSQTLSLTCTVSGFSFNTYAMNWIRQPPGKGLEWIARIRS |
| | KSNNYATYYAPSLKSRVTISVDTSKNQFSLKLSSVTAADTAVYYCVRHGDGN |
| | LWYIDVWGQGTLVTVSS (SEQ ID NO: 448) |
| Antibody 2H8 | Antibody 2H8 |
| 2H8V3-15 | EVQLVESGGGLVKPGGSLRLSCAASGFSFNTYAMNWVRQAPGKGLEWIARI |
| | RSKSNNYATYYAAPVKGRFTISRDDSKNTLYLQMNSLKTEDTAVYYCVRHG |
| | DGNLWYIDVWGQGTLVTVSS (SEQ ID NO: 440) |
| 2H8V3-48 | EVQLVESGGGLVQPGGSLRLSCAASGFSFNTYAMNWVRQAPGKGLEWIARI |
| | RSKSNNYATYYADSVKGRFTISRDNAKNSLYLQMNSLRAEDTAVYYCVRHG |
| | DGNLWYIDVWGQGTLVTVSS (SEQ ID NO: 441) |
| 2H8V3-23 | EVQLLESGGGLVQPGGSLRLSCAASGFSFNTYAMNWVRQAPGKGLEWIARI |
| | RSKSNNYATYYADSVKGRFTISRDNSKNTLYLQMNSLRAEDTAVYYCVRHG |
| | DGNLWYIDVWGQGTLVTVSS (SEQ ID NO: 442) |
| 2H8V3-7 | EVQLVESGGGLVQPGGSLRLSCAASGFSFNTYAMNWVRQAPGKGLEWIARI |
| | RSKSNNYATYYADSVKGRFTISRDNAKNSLYLQMNSLRAEDTAVYYCVRHG |
| | DGNLWYIDVWGQGTLVTVSS (SEQ ID NO: 441) |
| 2H8V3-30 | QVQLVESGGGVVQPGRSLRLSCAASGFSFNTYAMNWVRQAPGKGLEWIARI |
| | RSKSNNYATYYADSVKGRFTISRDNSKNTLYLQMNSLRAEDTAVYYCVRHG |
| | DGNLWYIDVWGQGTLVTVSS (SEQ ID NO: 443) |
| 2H8V5-51 | EVQLVQSGAEVKKPGESLKISCKGSGFSFNTYAMNWVRQMPGKGLEWIARI |
| | RSKSNNYATYYAPSFQGQVTISADKSISTAYLQWSSLKASDTAMYYCVRHG |
| | DGNLWYIDVWGQGTLVTVSS (SEQ ID NO: 445) |
| 2H8V1-69 | QVQLVQSGAEVKKPGSSVKVSCKASGFSFNTYAMNWVRQAPGQGLEWIARI |
| | RSKSNNYATYYAQKFQGRVTITADESTSTAYMELSSLRSEDTAVYYCVRHG |
| | DGNLWYIDVWGQGTLVTVSS (SEQ ID NO: 444) |
| 2H8V1-46 | QVQLVQSGAEVKKPGASVKVSCKASGFSFNTYAMNWVRQAPGQGLEWIAR |
| | IRSKSNNYATYYAQKFQGRVTMTRDTSTSTVYMELSSLRSEDTAVYYCVRH |
| | GDGNLWYIDVWGQGTLVTVSS (SEQ ID NO: 446) |
| 2H8V4-59 | QVQLQESGPGLVKPSETLSLTCTVSGFSFNTYAMNWIRQPPGKGLEWIARIRS |
| | KSNNYATYYAPSLKSRVTISVDTSKNQFSLKLSSVTAADTAVYYCVRHGDGN |
| | LWYIDVWGQGTLVTVSS (SEQ ID NO: 447) |
| 2H8V4-30-4 | QVQLQESGPGLVKPSQTLSLTCTVSGFSFNTYAMNWIRQPPGKGLEWIARIRS |
| | KSNNYATYYAPSLKSRVTISVDTSKNQFSLKLSSVTAADTAVYYCVRHGDGN |
| | LWYIDVWGQGTLVTVSS (SEQ ID NO: 448) |
| Antibody 3A2 | Antibody 3A2 |
| 3A2V5-51 | EVQLVQSGAEVKKPGESLKISCKGSGYPFSNFWITWVRQMPGKGLEWIGDIY |
| | PGSDNSNYNPSFQGQVTISADKSISTAYLQWSSLKASDTAMYYCAREAYYTN |
| | PGFAYWGQGTLVTVSS (SEQ ID NO: 462) |
| 3A2V1-69 | QVQLVQSGAEVKKPGSSVKVSCKASGYPFSNFWITWVRQAPGQGLEWIGDI |
| | YPGSDNSNYNQKFQGRVTITADESTSTAYMELSSLRSEDTAVYYCAREAYYT |
| | NPGFAYWGQGTLVTVSS (SEQ ID NO: 463) |
| 3A2V1-46 | QVQLVQSGAEVKKPGASVKVSCKASGYPFSNFWITWVRQAPGQGLEWIGDI |
| | YPGSDNSNYNQKFQGRVTMTRDTSTSTVYMELSSLRSEDTAVYYCAREAYY |
| | TNPGFAYWGQGTLVTVSS (SEQ ID NO: 464) |
| 3A2V3-48 | EVQLVESGGGLVQPGGSLRLSCAASGYPFSNFWITWVRQAPGKGLEWIGDIY |
| | PGSDNSNYNDSVKGRFTISRDNAKNSLYLQMNSLRAEDTAVYYCAREAYYT |
| | NPGFAYWGQGTLVTVSS (SEQ ID NO: 465) |
| 3A2V3-30 | QVQLVESGGGVVQPGRSLRLSCAASGYPFSNFWITWVRQAPGKGLEWIGDI |
| | YPGSDNSNYNDSVKGRFTISRDNSKNTLYLQMNSLRAEDTAVYYCAREAYY |
| | TNPGFAYWGQGTLVTVSS (SEQ ID NO: 466) |
| 3A2V3-7 | EVQLVESGGGLVQPGGSLRLSCAASGYPFSNFWITWVRQAPGKGLEWIGDIY |
| | PGSDNSNYNDSVKGRFTISRDNAKNSLYLQMNSLRAEDTAVYYCAREAYYT |
| | NPGFAYWGQGTLVTVSS (SEQ ID NO: 465) |
| 3A2V3-23 | EVQLLESGGGLVQPGGSLRLSCAASGYPFSNFWITWVRQAPGKGLEWIGDIY |
| | PGSDNSNYNDSVKGRFTISRDNSKNTLYLQMNSLRAEDTAVYYCAREAYYT |
| | NPGFAYWGQGTLVTVSS (SEQ ID NO: 467) |
| 3A2V4-59 | QVQLQESGPGLVKPSETLSLTCTVSGYPFSNFWITWIRQPPGKGLEWIGDIYP |
| | GSDNSNYNPSLKSRVTISVDTSKNQFSLKLSSVTAADTAVYYCAREAYYTNP |
| | GFAYWGQGTLVTVSS (SEQ ID NO: 468) |
| IGHV3-15 | EVQLVESGGGLVKPGGSLRLSCAASGYPFSNFWITWVRQAPGKGLEWIGDIY |
| | PGSDNSNYNAPVKGRFTISRDDSKNTLYLQMNSLKTEDTAVYYCAREAYYT |
| | NPGFAYWGQGTLVTVSS (SEQ ID NO: 469) |
| 3A2V4-39 | QLQLQESGPGLVKPSETLSLTCTVSGYPFSNFWITWIRQPPGKGLEWIGDIYP |
| | GSDNSNYNPSLKSRVTISVDTSKNQFSLKLSSVTAADTAVYYCAREAYYTNP |
| | GFAYWGQGTLVTVSS (SEQ ID NO: 470) |
| Antibody 3A7 | Antibody 3A7 |
| 3A7V3-15 | EVQLVESGGGLVKPGGSLRLSCAASGFTFSDAWMGWVRQAPGKGLEWVAE |
| | IRDKVKNHATYYAAPVKGRFTISRDDSKNTLYLQMNSLKTEDTAVYYCRLG |
| | VFDYWGQGTLVTVSS (SEQ ID NO: 430) |
| 3A7V3-7 | EVQLVESGGGLVQPGGSLRLSCAASGFTFSDAWMGWVRQAPGKGLEWVAE |
| | IRDKVKNHATYYADSVKGRFTISRDNAKNSLYLQMNSLRAEDTAVYYCRLG |
| | VFDYWGQGTLVTVSS (SEQ ID NO: 431) |
| 3A7V3-23 | EVQLLESGGGLVQPGGSLRLSCAASGFTFSDAWMGWVRQAPGKGLEWVAE |
| | IRDKVKNHATYYADSVKGRFTISRDNSKNTLYLQMNSLRAEDTAVYYCRLG |
| | VFDYWGQGTLVTVSS (SEQ ID NO: 432) |
| 3A7V3-48 | EVQLVESGGGLVQPGGSLRLSCAASGFTFSDAWMGWVRQAPGKGLEWVAE |
| | IRDKVKNHATYYADSVKGRFTISRDNAKNSLYLQMNSLRAEDTAVYYCRLG |
| | VFDYWGQGTLVTVSS (SEQ ID NO: 431) |
| 3A7V3-30 | QVQLVESGGGVVQPGRSLRLSCAASGFTFSDAWMGWVRQAPGKGLEWVAE |
| | IRDKVKNHATYYADSVKGRFTISRDNSKNTLYLQMNSLRAEDTAVYYCRLG |
| | VFDYWGQGTLVTVSS (SEQ ID NO: 433) |
| 3A7V1-69 | QVQLVQSGAEVKKPGSSVKVSCKASGFTFSDAWMGWVRQAPGQGLEWVA |
| | EIRDKVKNHATYYAQKFQGRVTITADESTSTAYMELSSLRSEDTAVYYCRLG |
| | VFDYWGQGTLVTVSS (SEQ ID NO: 434) |
| 3A7V1-46 | QVQLVQSGAEVKKPGASVKVSCKASGFTFSDAWMGWVRQAPGQGLEWVA |
| | EIRDKVKNHATYYAQKFQGRVTMTRDTSTSTVYMELSSLRSEDTAVYYCRL |
| | GVFDYWGQGTLVTVSS (SEQ ID NO: 435) |
| 3A7V5-51 | EVQLVQSGAEVKKPGESLKISCKGSGFTFSDAWMGWVRQMPGKGLEWVAE |
| | IRDKVKNHATYYAPSFQGQVTISADKSISTAYLQWSSLKASDTAMYYCRLG |
| | VFDYWGQGTLVTVSS (SEQ ID NO: 436) |
| 3A7V4-59 | QVQLQESGPGLVKPSETLSLTCTVSGFTFSDAWMGWIRQPPGKGLEWVAEIR |
| | DKVKNHATYYAPSLKSRVTISVDTSKNQFSLKLSSVTAADTAVYYCRLGVFD |
| | YWGQGTLVTVSS (SEQ ID NO: 437) |
| 3A7V4-39 | QLQLQESGPGLVKPSETLSLTCTVSGFTFSDAWMGWIRQPPGKGLEWVAEIR |
| | DKVKNHATYYAPSLKSRVTISVDTSKNQFSLKLSSVTAADTAVYYCRLGVFD |
| | YWGQGTLVTVSS (SEQ ID NO: 438) |
| Antibody 3B10 | Antibody 3B10 |
| 3B10V3-15 | EVQLVESGGGLVKPGGSLRLSCAASGLTSNTYTQTWVRQAPGKGLEWESVI |
| | RSKSNNFSTLYAAPVKGRFTISRDDSKNTLYLQMNSLKTEDTAVYYCVRHKS |
| | NRYPGVYWGQGTLVTVSS (SEQ ID NO: 472) |
| 3B10V3-30 | QVQLVESGGGVVQPGRSLRLSCAASGLTSNTYTQTWVRQAPGKGLEWESVI |
| | RSKSNNFSTLYADSVKGRFTISRDNSKNTLYLQMNSLRAEDTAVYYCVRHKS |
| | NRYPGVYWGQGTLVTVSS (SEQ ID NO: 473) |
| 3B10V3-23 | EVQLLESGGGLVQPGGSLRLSCAASGLTSNTYTQTWVRQAPGKGLEWESVI |
| | RSKSNNFSTLYADSVKGRFTISRDNSKNTLYLQMNSLRAEDTAVYYCVRHKS |
| | NRYPGVYWGQGTLVTVSS (SEQ ID NO: 474) |
| 3B10V1-46 | QVQLVQSGAEVKKPGASVKVSCKASGLTSNTYTQTWVRQAPGQGLEWESVI |
| | RSKSNNFSTLYAQKFQGRVTMTRDTSTSTVYMELSSLRSEDTAVYYCVRHK |
| | SNRYPGVYWGQGTLVTVSS (SEQ ID NO: 475) |
| 3B10V3-48 | EVQLVESGGGLVQPGGSLRLSCAASGLTSNTYTQTWVRQAPGKGLEWESVI |
| | RSKSNNESTLYADSVKGRFTISRDNAKNSLYLQMNSLRAEDTAVYYCVRHK |
| | SNRYPGVYWGQGTLVTVSS (SEQ ID NO: 476) |
| 3B10V1-69 | QVQLVQSGAEVKKPGSSVKVSCKASGLTSNTYTQTWVRQAPGQGLEWESVI |
| | RSKSNNFSTLYAQKFQGRVTITADESTSTAYMELSSLRSEDTAVYYCVRHKS |
| | NRYPGVYWGQGTLVTVSS (SEQ ID NO: 477) |
| 3B10V3-7 | EVQLVESGGGLVQPGGSLRLSCAASGLTSNTYTQTWVRQAPGKGLEWESVI |
| | RSKSNNFSTLYADSVKGRFTISRDNAKNSLYLQMNSLRAEDTAVYYCVRHK |
| | SNRYPGVYWGQGTLVTVSS (SEQ ID NO: 476) |
| 3B10V5-51 | EVQLVQSGAEVKKPGESLKISCKGSGLTSNTYTQTWVRQMPGKGLEWESVI |
| | RSKSNNFSTLYAPSFQGQVTISADKSISTAYLQWSSLKASDTAMYYCVRHKS |
| | NRYPGVYWGQGTLVTVSS (SEQ ID NO: 478) |
| 3B10V4-59 | QVQLQESGPGLVKPSETLSLTCTVSGLTSNTYTQTWIRQPPGKGLEWESVIRS |
| | KSNNFSTLYAPSLKSRVTISVDTSKNQFSLKLSSVTAADTAVYYCVRHKSNR |
| | YPGVYWGQGTLVTVSS (SEQ ID NO: 479) |
| 3B10V4-39 | QLQLQESGPGLVKPSETLSLTCTVSGLTSNTYTQTWIRQPPGKGLEWESVIRS |
| | KSNNFSTLYAPSLKSRVTISVDTSKNQFSLKLSSVTAADTAVYYCVRHKSNR |
| | YPGVYWGQGTLVTVSS (SEQ ID NO: 480) |
| Antibody 4F11 | Antibody 4F11 |
| 4F11V5-51 | EVQLVQSGAEVKKPGESLKISCKGSGYPFSNFWITWVRQMPGKGLEWIGDIY |
| | PGSDNSNYNPSFQGQVTISADKSISTAYLQWSSLKASDTAMYYCAREAYYTN |
| | PGFAYWGQGTLVTVSS (SEQ ID NO: 462) |
| 4F11V1-69 | QVQLVQSGAEVKKPGSSVKVSCKASGYPFSNFWITWVRQAPGQGLEWIGDI |
| | YPGSDNSNYNQKFQGRVTITADESTSTAYMELSSLRSEDTAVYYCAREAYYT |
| | NPGFAYWGQGTLVTVSS (SEQ ID NO: 463) |
| 4F11V1-46 | QVQLVQSGAEVKKPGASVKVSCKASGYPFSNFWITWVRQAPGQGLEWIGDI |
| | YPGSDNSNYNQKFQGRVTMTRDTSTSTVYMELSSLRSEDTAVYYCAREAYY |
| | TNPGFAYWGQGTLVTVSS (SEQ ID NO: 464) |
| 4F11V3-48 | EVQLVESGGGLVQPGGSLRLSCAASGYPFSNFWITWVRQAPGKGLEWIGDIY |
| | PGSDNSNYNDSVKGRFTISRDNAKNSLYLQMNSLRAEDTAVYYCAREAYYT |
| | NPGFAYWGQGTLVTVSS (SEQ ID NO: 465) |
| 4F11V3-30 | QVQLVESGGGVVQPGRSLRLSCAASGYPFSNFWITWVRQAPGKGLEWIGDI |
| | YPGSDNSNYNDSVKGRFTISRDNSKNTLYLQMNSLRAEDTAVYYCAREAYY |
| | TNPGFAYWGQGTLVTVSS (SEQ ID NO: 466) |
| 4F11V3-7 | EVQLVESGGGLVQPGGSLRLSCAASGYPFSNFWITWVRQAPGKGLEWIGDIY |
| | PGSDNSNYNDSVKGRFTISRDNAKNSLYLQMNSLRAEDTAVYYCAREAYYT |
| | NPGFAYWGQGTLVTVSS (SEQ ID NO: 465) |
| 4F11V3-23 | EVQLLESGGGLVQPGGSLRLSCAASGYPFSNFWITWVRQAPGKGLEWIGDIY |
| | PGSDNSNYNDSVKGRFTISRDNSKNTLYLQMNSLRAEDTAVYYCAREAYYT |
| | NPGFAYWGQGTLVTVSS (SEQ ID NO: 467) |
| 4F11V4-59 | QVQLQESGPGLVKPSETLSLTCTVSGYPFSNFWITWIRQPPGKGLEWIGDIYP |
| | GSDNSNYNPSLKSRVTISVDTSKNQFSLKLSSVTAADTAVYYCAREAYYTNP |
| | GFAYWGQGTLVTVSS (SEQ ID NO: 468) |
| 4F11V3-15 | EVQLVESGGGLVKPGGSLRLSCAASGYPFSNFWITWVRQAPGKGLEWIGDIY |
| | PGSDNSNYNAPVKGRFTISRDDSKNTLYLQMNSLKFEDTAVYYCAREAYYT |
| | NPGFAYWGQGTLVTVSS (SEQ ID NO: 469) |
| 4F11V4-39 | QLQLQESGPGLVKPSETLSLTCTVSGYPFSNFWITWIRQPPGKGLEWIGDIYP |
| | GSDNSNYNPSLKSRVTISVDTSKNQFSLKLSSVTAADTAVYYCAREAYYTNP |
| | GFAYWGQGTLVTVSS (SEQ ID NO: 470) |
| Antibody 6H6 | Antibody 6H6 |
| 6H6V3-15 | EVQLVESGGGLVKPGGSLRLSCAASGFTFSDAWMDWVRQAPGKGLEWWV |
| | AEIRNKVNNHATYYAPVKGRFTISRDDSKNTLYLQMNSLKTEDTAVYYCCT |
| | SLYDGYYLRFAWGQGTLVTVSS (SEQ ID NO: 482) |
| 6H6V3-7 | EVQLVESGGGLVQPGGSLRLSCAASGFTFSDAWMDWVRQAPGKGLEWWV |
| | AEIRNKVNNHATYYDSVKGRFTISRDNAKNSLYLQMNSLRAEDTAVYYCCT |
| | SLYDGYYLRFAWGQGTLVTVSS (SEQ ID NO: 483) |
| 6H6V3-23 | EVQLLESGGGLVQPGGSLRLSCAASGFTFSDAWMDWVRQAPGKGLEWWVA |
| | EIRNKVNNHATYYDSVKGRFTISRDNSKNTLYLQMNSLRAEDTAVYYCCTS |
| | LYDGYYLRFAWGQGTLVTVSS (SEQ ID NO: 484) |
| 6H6V3-48 | EVQLVESGGGLVQPGGSLRLSCAASGFTFSDAWMDWVRQAPGKGLEWWV |
| | AEIRNKVNNHATYYDSVKGRFTISRDNAKNSLYLQMNSLRAEDTAVYYCCT |
| | SLYDGYYLRFAWGQGTLVTVSS (SEQ ID NO: 483) |
| 6H6V3-30 | QVQLVESGGGVVQPGRSLRLSCAASGFTFSDAWMDWVRQAPGKGLEWWV |
| | AEIRNKVNNHATYYDSVKGRFTISRDNSKNTLYLQMNSLRAEDTAVYYCCT |
| | SLYDGYYLRFAWGQGTLVTVSS (SEQ ID NO: 485) |
| 6H6V1-46 | QVQLVQSGAEVKKPGASVKVSCKASGFTFSDAWMDWVRQAPGQGLEWWV |
| | AEIRNKVNNHATYYQKFQGRVTMTRDTSTSTVYMELSSLRSEDTAVYYCCT |
| | SLYDGYYLRFAWGQGTLVTVSS (SEQ ID NO: 486) |
| 6H6V1-69 | QVQLVQSGAEVKKPGSSVKVSCKASGFTFSDAWMDWVRQAPGQGLEWWV |
| | AEIRNKVNNHATYYQKFQGRVTITADESTSTAYMELSSLRSEDTAVYYCCTS |
| | LYDGYYLRFAWGQGTLVTVSS (SEQ ID NO: 487) |
| 6H6V5-51 | EVQLVQSGAEVKKPGESLKISCKGSGFTFSDAWMDWVRQMPGKGLEWWVA |
| | EIRNKVNNHATYYPSFQGQVTISADKSISTAYLQWSSLKASDTAMYYCCTSL |
| | YDGYYLRFAWGQGTLVTVSS (SEQ ID NO: 488) |
| 6H6V4-59 | QVQLQESGPGLVKPSETLSLTCTVSGFTFSDAWMDWIRQPPGKGLEWWVAE |
| | IRNKVNNHATYYPSLKSRVTISVDTSKNQFSLKLSSVTAADTAVYYCCTSLY |
| | DGYYLRFAWGQGTLVTVSS (SEQ ID NO: 489) |
| 6H6V4-39 | QLQLQESGPGLVKPSETLSLTCTVSGFTFSDAWMDWIRQPPGKGLEWWVAEI |
| | RNKVNNHATYYPSLKSRVTISVDTSKNQFSLKLSSVTAADTAVYYCCTSLYD |
| | GYYLRFAWGQGTLVTVSS (SEQ ID NO: 490) |
| Antibody7A9 | Antibody 7A9 |
| 7A9V3-15 | EVQLVESGGGLVKPGGSLRLSCAASGFTFNTYSMNWVRQAPGKGLEWVAHI |
| | KTKZNNFATFYAAPVKGRFTISRDDSKNTLYLQMNSLKTEDTAVYYCVZHZ |
| | SNNYPFAYWGQGTLVTVSS (SEQ ID NO: 492) |
| 7A9V3-48 | EVQLVESGGGLVQPGGSLRLSCAASGFTFNTYSMNWVRQAPGKGLEWVAHI |
| | KTKZNNFATFYADSVKGRFTISRDNAKNSLYLQMNSLRAEDTAVYYCVZHZ |
| | SNNYPFAYWGQGTLVTVSS (SEQ ID NO: 493) |
| 7A9V3-23 | EVQLLESGGGLVQPGGSLRLSCAASGFTFNTYSMNWVRQAPGKGLEWVAHI |
| | KTKZNNFATFYADSVKGRFTISRDNSKNTLYLQMNSLRAEDTAVYYCVZHZ |
| | SNNYPFAYWGQGTLVTVSS (SEQ ID NO: 494) |
| 7A9V3-7 | EVQLVESGGGLVQPGGSLRLSCAASGFTFNTYSMNWVRQAPGKGLEWVAHI |
| | KTKZNNFATFYADSVKGRFTISRDNAKNSLYLQMNSLRAEDTAVYYCVZHZ |
| | SNNYPFAYWGQGTLVTVSS (SEQ ID NO: 493) |
| 7A9V3-30 | QVQLVESGGGVVQPGRSLRLSCAASGFTFNTYSMNWVRQAPGKGLEWVAH |
| | IKTKZNNFATFYADSVKGRFTISRDNSKNTLYLQMNSLRAEDTAVYYCVZH |
| | ZSNNYPFAYWGQGTLVTVSS (SEQ ID NO: 495) |
| 7A9V1-46 | QVQLVQSGAEVKKPGASVKVSCKASGFTFNTYSMNWVRQAPGQGLEWVAH |
| | IKTKZNNFATFYAQKFQGRVTMTRDTSTSTVYMELSSLRSEDTAVYYCVZH |
| | ZSNNYPFAYWGQGTLVTVSS (SEQ ID NO: 496) |
| 7A9V1-69 | QVQLVQSGAEVKKPGSSVKVSCKASGFTFNTYSMNWVRQAPGQGLEWVAH |
| | IKTKZNNFATFYAQKFQGRVTITADESTSTAYMELSSLRSEDTAVYYCVZHZ |
| | SNNYPFAYWGQGTLVTVSS (SEQ ID NO: 497) |
| 7A9V5-51 | EVQLVQSGAEVKKPGESLKISCKGSGFTFNTYSMNWVRQMPGKGLEWVAHI |
| | KTKZNNFATFYAPSFQGQVTISADKSISTAYLQWSSLKASDTAMYYCVZHZS |
| | NNYPFAYWGQGTLVTVSS (SEQ ID NO: 498) |
| 7A9V4-59 | QVQLQESGPGLVKPSETLSLTCTVSGFTFNTYSMNWIRQPPGKGLEWVAHIK |
| | TKZNNFATFYAPSLKSRVTISVDTSKNQFSLKLSSVTAADTAVYYCVZHZSN |
| | NYPFAYWGQGTLVTVSS (SEQ ID NO: 499) |
| 7A9V4-30-4 | QVQLQESGPGLVKPSQTLSLTCTVSGFTFNTYSMNWIRQPPGKGLEWVAHIK |
| | TKZNNFATFYAPSLKSRVTISVDTSKNQFSLKLSSVTAADTAVYYCVZHZSN |
| | NYPFAYWGQGTLVTVSS (SEQ ID NO: 500) |
| Antibody 7B3 | Antibody 7B3 |
| 7B3V1-46 | QVQLVQSGAEVKKPGASVKVSCKASGYTFTTYWIHWVRQAPGQGLEWIGR |
| | NDPNSGGSNYNQKFQGRVTMTRDTSTSTVYMELSSLRSEDTAVYYCVRTNW |
| | DGDFWGQGTLVTVSS (SEQ ID NO: 502) |
| 7B3V5-51 | EVQLVQSGAEVKKPGESLKISCKGSGYTFTTYWIHWVRQMPGKGLEWIGRN |
| | DPNSGGSNYNPSFQGQVTISADKSISTAYLQWSSLKASDTAMYYCVRTNWD |
| | GDFWGQGTLVTVSS (SEQ ID NO: 503) |
| 7B3V1-69 | QVQLVQSGAEVKKPGSSVKVSCKASGYTFTTYWIHWVRQAPGQGLEWIGR |
| | NDPNSGGSNYNQKFQGRVTITADESTSTAYMELSSLRSEDTAVYYCVRTNW |
| | DGDFWGQGTLVTVSS (SEQ ID NO: 504) |
| 7B3V3-23 | EVQLLESGGGLVQPGGSLRLSCAASGYTFTTYWIHWVRQAPGKGLEWIGRN |
| | DPNSGGSNYNDSVKGRFTISRDNSKNTLYLQMNSLRAEDTAVYYCVRTNWD |
| | GDFWGQGTLVTVSS (SEQ ID NO: 505) |
| 7B3V3-7 | EVQLVESGGGLVQPGGSLRLSCAASGYTFTTYWIHWVRQAPGKGLEWIGRN |
| | DPNSGGSNYNDSVKGRFTISRDNAKNSLYLQMNSLRAEDTAVYYCVRTNWD |
| | GDFWGQGTLVTVSS (SEQ ID NO: 506) |
| 7B3V3-30 | QVQLVESGGGVVQPGRSLRLSCAASGYTFTTYWIHWVRQAPGKGLEWIGR |
| | NDPNSGGSNYNDSVKGRFTISRDNSKNTLYLQMNSLRAEDTAVYYCVRTNW |
| | DGDFWGQGTLVTVSS (SEQ ID NO: 507) |
| 7B3V3-48 | EVQLVESGGGLVQPGGSLRLSCAASGYTFTTYWIHWVRQAPGKGLEWIGRN |
| | DPNSGGSNYNDSVKGRFTISRDNAKNSLYLQMNSLRAEDTAVYYCVRTNWD |
| | GDFWGQGTLVTVSS (SEQ ID NO: 506) |
| 7B3V4-59 | QVQLQESGPGLVKPSETLSLTCTVSGYTFTTYWIHWIRQPPGKGLEWIGRND |
| | PNSGGSNYNPSLKSRVTISVDTSKNQFSLKLSSVTAADTAVYYCVRTNWDGD |
| | FWGQGTLVTVSS (SEQ ID NO: 508) |
| 7B3V3-15 | EVQLVESGGGLVKPGGSLRLSCAASGYTFTTYWIHWVRQAPGKGLEWIGRN |
| | DPNSGGSNYNAPVKGRFTISRDDSKNTLYLQMNSLKTEDTAVYYCVRTNWD |
| | GDFWGQGTLVTVSS (SEQ ID NO: 509) |
| 7B3V4-30-4 | QVQLQESGPGLVKPSQTLSLTCTVSGYTFTTYWIHWIRQPPGKGLEWIGRND |
| | PNSGGSNYNPSLKSRVTISVDTSKNQFSLKLSSVTAADTAVYYCVRTNWDGD |
| | FWGQGTLVTVSS (SEQ ID NO: 510) |
| Antibody 8A1 | Antibody 8A1 |
| 8A1V5-51 | EVQLVQSGAEVKKPGESLKISCKGSGYAFSNYWMSWVRQMPGKGLEWIGQI |
| | YPGDGDTKYNPSFQGQVTISADKSISTAYLQWSSLKASDTAMYYCSREKGA |
| | DYYGSTYSAWFSYWGQGTLVTVSS (SEQ ID NO: 512) |
| 8A1V1-46 | QVQLVQSGAEVKKPGASVKVSCKASGYAFSNYWMSWVRQAPGQGLEWIG |
| | QIYPGDGDTKYNQKFQGRVTMTRDTSTSTVYMELSSLRSEDTAVYYCSREK |
| | GADYYGSTYSAWFSYWGQGTLVTVSS (SEQ ID NO: 513) |
| 8A1V3-23 | EVQLLESGGGLVQPGGSLRLSCAASGYAFSNYWMSWVRQAPGKGLEWIGQI |
| | YPGDGDTKYNDSVKGRFTISRDNSKNTLYLQMNSLRAEDTAVYYCSREKGA |
| | DYYGSTYSAWFSYWGQGTLVTVSS (SEQ ID NO: 514) |
| 8A1V1-69 | QVQLVQSGAEVKKPGSSVKVSCKASGYAFSNYWMSWVRQAPGQGLEWIGQ |
| | IYPGDGDTKYNQKFQGRVTITADESTSTAYMELSSLRSEDTAVYYCSREKGA |
| | DYYGSTYSAWFSYWGQGTLVTVSS (SEQ ID NO: 515) |
| 8A1V3-7 | EVQLVESGGGLVQPGGSLRLSCAASGYAFSNYWMSWVRQAPGKGLEWIGQI |
| | YPGDGDTKYNDSVKGRFTISRDNAKNSLYLQMNSLRAEDTAVYYCSREKGA |
| | DYYGSTYSAWFSYWGQGTLVTVSS (SEQ ID NO: 516) |
| 8A1V3-48 | EVQLVESGGGLVQPGGSLRLSCAASGYAFSNYWMSWVRQAPGKGLEWIGQI |
| | YPGDGDTKYNDSVKGRFTISRDNAKNSLYLQMNSLRAEDTAVYYCSREKGA |
| | DYYGSTYSAWFSYWGQGTLVTVSS (SEQ ID NO: 516) |
| 8A1V3-30 | QVQLVESGGGVVQPGRSLRLSCAASGYAFSNYWMSWVRQAPGKGLEWIGQ |
| | IYPGDGDTKYNDSVKGRFTISRDNSKNTLYLQMNSLRAEDTAVYYCSREKG |
| | ADYYGSTYSAWFSYWGQGTLVTVSS (SEQ ID NO: 517) |
| 8A1V4-59 | QVQLQESGPGLVKPSETLSLTCTVSGYAFSNYWMSWIRQPPGKGLEWIGQIY |
| | PGDGDTKYNPSLKSRVTISVDTSKNQFSLKLSSVTAADTAVYYCSREKGADY |
| | YGSTYSAWFSYWGQGTLVTVSS (SEQ ID NO: 518) |
| 8A1V3-15 | EVQLVESGGGLVKPGGSLRLSCAASGYAFSNYWMSWVRQAPGKGLEWIGQI |
| | YPGDGDTKYNAPVKGRFTISRDDSKNTLYLQMNSLKTEDTAVYYCSREKGA |
| | DYYGSTYSAWFSYWGQGTLVTVSS (SEQ ID NO: 519) |
| 8A1V4-30-4 | QVQLQESGPGLVKPSQTLSLTCTVSGYAFSNYWMSWIRQPPGKGLEWIGQIY |
| | PGDGDTKYNPSLKSRVTISVDTSKNQFSLKLSSVTAADTAVYYCSREKGADY |
| | YGSTYSAWFSYWGQGTLVTVSS (SEQ ID NO: 520) |
| Antibody 9F5 | Antibody 9F5 |
| 9F5V5-51 | EVQLVQSGAEVKKPGESLKISCKGSGYAFSSSWMNWVRQMPGKGLEWIGRI |
| | YPGDGDTNYNPSFQGQVTISADKSISTAYLQWSSLKASDTAMYYCARLLRN |
| | QPGESYAMDYWGQGTLVTVSS (SEQ ID NO: 522) |
| 9F5V1-46 | QVQLVQSGAEVKKPGASVKVSCKASGYAFSSSWMNWVRQAPGQGLEWIGR |
| | IYPGDGDTNYNQKFQGRVTMTRDTSTSTVYMELSSLRSEDTAVYYCARLLR |
| | NQPGESYAMDYWGQGTLVTVSS (SEQ ID NO: 523) |
| 9F5V1-69 | QVQLVQSGAEVKKPGSSVKVSCKASGYAFSSSWMNWVRQAPGQGLEWIGR |
| | IYPGDGDTNYNQKFQGRVTITADESTSTAYMELSSLRSEDTAVYYCARLLRN |
| | QPGESYAMDYWGQGTLVTVSS (SEQ ID NO: 524) |
| 9F5V3-23 | EVQLLESGGGLVQPGGSLRLSCAASGYAFSSSWMNWVRQAPGKGLEWIGRI |
| | YPGDGDTNYNDSVKGRFTISRDNSKNTLYLQMNSLRAEDTAVYYCARLLRN |
| | QPGESYAMDYWGQGTLVTVSS (SEQ ID NO: 525) |
| 9F5V3-7 | EVQLVESGGGLVQPGGSLRLSCAASGYAFSSSWMNWVRQAPGKGLEWIGRI |
| | YPGDGDTNYNDSVKGRFTISRDNAKNSLYLQMNSLRAEDTAVYYCARLLRN |
| | QPGESYAMDYWGQGTLVTVSS (SEQ ID NO: 526) |
| 9F5V3-48 | EVQLVESGGGLVQPGGSLRLSCAASGYAFSSSWMNWVRQAPGKGLEWIGRI |
| | YPGDGDTNYNDSVKGRFTISRDNAKNSLYLQMNSLRAEDTAVYYCARLLRN |
| | QPGESYAMDYWGQGTLVTVSS (SEQ ID NO: 526) |
| 9F5V3-30 | QVQLVESGGGVVQPGRSLRLSCAASGYAFSSSWMNWVRQAPGKGLEWIGRI |
| | YPGDGDTNYNDSVKGRFTISRDNSKNTLYLQMNSLRAEDTAVYYCARLLRN |
| | QPGESYAMDYWGQGTLVTVSS (SEQ ID NO: 527) |
| 9F5V4-59 | QVQLQESGPGLVKPSETLSLTCTVSGYAFSSSWMNWIRQPPGKGLEWIGRIY |
| | PGDGDTNYNPSLKSRVTISVDTSKNQFSLKLSSVTAADTAVYYCARLLRNQP |
| | GESYAMDYWGQGTLVTVSS (SEQ ID NO: 528) |
| 9F5V3-15 | EVQLVESGGGLVKPGGSLRLSCAASGYAFSSSWMNWVRQAPGKGLEWIGRI |
| | YPGDGDTNYNAPVKGRFTISRDDSKNTLYLQMNSLKTEDTAVYYCARLLRN |
| | QPGESYAMDYWGQGTLVTVSS (SEQ ID NO: 529) |
| 9F5V4-30-4 | QVQLQESGPGLVKPSQTLSLTCTVSGYAFSSSWMNWIRQPPGKGLEWIGRIY |
| | PGDGDTNYNPSLKSRVTISVDTSKNQFSLKLSSVTAADTAVYYCARLLRNQP |
| | GESYAMDYWGQGTLVTVSS (SEQ ID NO: 530) |
| 9F5-H1 | QVQLVQSGAEVKKPGASVKVSCKASGYAFSSSWMNWVRQAPGQGLEWMG |
| | RIYPGDGDTNYAQKFQGRVTMTRDTSTSTVYMELSSLRSEDTAVYYCARLL |
| | RNQPGESYAMDYWGQGTLVTVSS (SEQ ID NO: 845) |
| 9F5-H2 | QVQLVQSGAEVKKPGASVKVSCKASGYAFSSSWMNWVRQAPGQGLEWIGR |
| | IYPGDGDTNYAQKFQGRVTMTADTSTSTVYMELSSLRSEDTAVYYCARLLR |
| | NQPGESYAMDYWGQGTLVTVSS (SEQ ID NO: 846) |
| 9F5-H3 | QVQLVQSGAEVKKPGASLKISCKASGYAFSSSWMNWVRQAPGQGLEWIGRI |
| | YPGDGDTNYAQKFQGRATLTADTSTSTAYMELSSLRSEDTAVYYCARLLRN |
| | QPGESYAMDYWGQGALVTVSS (SEQ ID NO: 847) |
| Antibody 9G1 | Antibody 9G1 |
| 9G1V5-51 | EVQLVQSGAEVKKPGESLKISCKGSGYIFTTYWIHWVRQMPGKGLEWIGRID |
| | PNNGDTNYNPSFQGQVTISADKSISTAYLQWSSLKASDTAMYYCVMTGTDF |
| | DYWGQGTLVTVSS (SEQ ID NO: 532) |
| 9G1V1-46 | QVQLVQSGAEVKKPGASVKVSCKASGYIFTTYWIHWVRQAPGQGLEWIGRI |
| | DPNNGDTNYNQKFQGRVTMTRDTSTSTVYMELSSLRSEDTAVYYCVMTGT |
| | DFDYWGQGTLVTVSS (SEQ ID NO: 533) |
| 9G1V1-69 | QVQLVQSGAEVKKPGSSVKVSCKASGYIFTTYWIHWVRQAPGQGLEWIGRI |
| | DPNNGDTNYNQKFQGRVTITADESTSTAYMELSSLRSEDTAVYYCVMTGTD |
| | FDYWGQGTLVTVSS (SEQ ID NO: 534) |
| 9G1V3-23 | EVQLLESGGGLVQPGGSLRLSCAASGYIFTTYWIHWVRQAPGKGLEWIGRID |
| | PNNGDTNYNDSVKGRFTISRDNSKNTLYLQMNSLRAEDTAVYYCVMTGTDF |
| | DYWGQGTLVTVSS (SEQ ID NO: 535) |
| 9G1V3-30 | QVQLVESGGGVVQPGRSLRLSCAASGYIFTTYWIHWVRQAPGKGLEWIGRI |
| | DPNNGDTNYNDSVKGRFTISRDNSKNTLYLQMNSLRAEDTAVYYCVMTGT |
| | DFDYWGQGTLVTVSS (SEQ ID NO: 536) |
| 9G1V3-7 | EVQLVESGGGLVQPGGSLRLSCAASGYIFTTYWIHWVRQAPGKGLEWIGRID |
| | PNNGDTNYNDSVKGRFTISRDNAKNSLYLQMNSLRAEDTAVYYCVMTGTD |
| | FDYWGQGTLVTVSS (SEQ ID NO: 537) |
| 9G1V3-48 | EVQLVESGGGLVQPGGSLRLSCAASGYIFTTYWIHWVRQAPGKGLEWIGRID |
| | PNNGDTNYNDSVKGRFTISRDNAKNSLYLQMNSLRAEDTAVYYCVMTGTD |
| | FDYWGQGTLVTVSS (SEQ ID NO: 537) |
| 9G1V4-59 | QVQLQESGPGLVKPSETLSLTCTVSGYIFTTYWIHWIRQPPGKGLEWIGRIDP |
| | NNGDTNYNPSLKSRVTISVDTSKNQFSLKLSSVTAADTAVYYCVMTGTDFD |
| | YWGQGTLVTVSS (SEQ ID NO: 538) |
| 9G1V3-15 | EVQLVESGGGLVKPGGSLRLSCAASGYIFTTYWIHWVRQAPGKGLEWIGRID |
| | PNNGDTNYNAPVKGRFTISRDD SKNTLYLQMNSLKTEDTAVYYCVMTGTDF |
| | DYWGQGTLVTVSS (SEQ ID NO: 539) |
| 9G1V4-30-4 | QVQLQESGPGLVKPSQTLSLTCTVSGYIFTTYWIHWIRQPPGKGLEWIGRIDP |
| | NNGDTNYNPSLKSRVTISVDTSKNQFSLKLSSVTAADTAVYYCVMTGTDFD |
| | YWGQGTLVTVSS (SEQ ID NO: 540) |
| Antibody 9G3 | Antibody 9G3 |
| 9G3V3-15 | EVQLVESGGGLVKPGGSLRLSCAASGFNFNTYAMKWVRQAPGKGLEWIARI |
| | RSNSNDYATNYSAPVKGRFTISRDDSKNTLYLQMNSLKTEDTAVYYCVGHKI |
| | NNYPFAHWGQGTLVTVSS (SEQ ID NO: 636) |
| 9G3V3-23 | EVQLLESGGGLVQPGGSLRLSCAASGFNFNTYAMKWVRQAPGKGLEWIARI |
| | RSNSNDYATNYSDSVKGRFTISRDNSKNTLYLQMNSLRAEDTAVYYCVGHKI |
| | NNYPFAHWGQGTLVTVSS (SEQ ID NO: 637) |
| 9G3V3-30 | QVQLVESGGGVVQPGRSLRLSCAASGFNFNTYAMKWVRQAPGKGLEWIARI |
| | RSNSNDYATNYSDSVKGRFTISRDNSKNTLYLQMNSLRAEDTAVYYCVGHKI |
| | NNYPFAHWGQGTLVTVSS (SEQ ID NO: 638) |
| 9G3V3-48 | EVQLVESGGGLVQPGGSLRLSCAASGFNFNTYAMKWVRQAPGKGLEWIARI |
| | RSNSNDYATNYSDSVKGRFTISRDNAKNSLYLQMNSLRAEDTAVYYCVGHK |
| | INNYPFAHWGQGTLVTVSS (SEQ ID NO: 639) |
| 9G3V3-7 | EVQLVESGGGLVQPGGSLRLSCAASGFNFNTYAMKWVRQAPGKGLEWIARI |
| | RSNSNDYATNYSDSVKGRFTISRDNAKNSLYLQMNSLRAEDTAVYYCVGHK |
| | INNYPFAHWGQGTLVTVSS (SEQ ID NO: 640) |
| 9G3V1-69 | QVQLVQSGAEVKKPGSSVKVSCKASGFNFNTYAMKWVRQAPGQGLEWIAR |
| | IRSNSNDYATNYSQKFQGRVTITADESTSTAYMELSSLRSEDTAVYYCVGHKI |
| | NNYPFAHWGQGTLVTVSS (SEQ ID NO: 641) |
| 9G3V1-46 | QVQLVQSGAEVKKPGASVKVSCKASGFNFNTYAMKWVRQAPGQGLEWIAR |
| | IRSNSNDYATNYSQKFQGRVTMTRDTSTSTVYMELSSLRSEDTAVYYCVGH |
| | KINNYPFAHWGQGTLVTVSS (SEQ ID NO: 642) |
| 9G3V5-51 | EVQLVQSGAEVKKPGESLKISCKGSGFNFNTYAMKWVRQMPGKGLEWIARI |
| | RSNSNDYATNYSPSFQGQVTISADKSISTAYLQWSSLKASDTAMYYCVGHKI |
| | NNYPFAHWGQGTLVTVSS (SEQ ID NO: 643) |
| 9G3V4-59 | QVQLQESGPGLVKPSETLSLTCTVSGFNFNTYAMKWIRQPPGKGLEWIARIR |
| | SNSNDYATNYSPSLKSRVTISVDTSKNQFSLKLSSVTAADTAVYYCVGHKINN |
| | YPFAHWGQGTLVTVSS (SEQ ID NO: 644) |
| 9G3V4-30-4 | QVQLQESGPGLVKPSQTLSLTCTVSGFNFNTYAMKWIRQPPGKGLEWIARIR |
| | SNSNDYATNYSPSLKSRVTISVDTSKNQFSLKLSSVTAADTAVYYCVGHKINN |
| | YPFAHWGQGTLVTVSS (SEQ ID NO: 645) |
| Antibody 10A9 | Antibody 10A9 |
| 10A9V5-51 | EVQLVQSGAEVKKPGESLKISCKGSGYPFSNFWITWVRQMPGKGLEWIGDIY |
| | PGSDNRNFNPSFQGQVTISADKSISTAYLQWSSLKASDTAMYYCAREAYYTN |
| | PGFAYWGQGTLVTVSS (SEQ ID NO: 542) |
| 10A9V1-69 | QVQLVQSGAEVKKPGSSVKVSCKASGYPFSNFWITWVRQAPGQGLEWIGDI |
| | YPGSDNRNFNQKFQGRVTITADESTSTAYMELSSLRSEDTAVYYCAREAYYT |
| | NPGFAYWGQGTLVTVSS (SEQ ID NO: 543) |
| 10A9V1-46 | QVQLVQSGAEVKKPGASVKVSCKASGYPFSNFWITWVRQAPGQGLEWIGDI |
| | YPGSDNRNFNQKFQGRVTMTRDTSTSTVYMELSSLRSEDTAVYYCAREAYY |
| | TNPGFAYWGQGTLVTVSS (SEQ ID NO: 544) |
| 10A9V3-48 | EVQLVESGGGLVQPGGSLRLSCAASGYPFSNFWITWVRQAPGKGLEWIGDIY |
| | PGSDNRNFNDSVKGRFTISRDNAKNSLYLQMNSLRAEDTAVYYCAREAYYT |
| | NPGFAYWGQGTLVTVSS (SEQ ID NO: 545) |
| 10A9V3-7 | EVQLVESGGGLVQPGGSLRLSCAASGYPFSNFWITWVRQAPGKGLEWIGDIY |
| | PGSDNRNFNDSVKGRFTISRDNAKNSLYLQMNSLRAEDTAVYYCAREAYYT |
| | NPGFAYWGQGTLVTVSS (SEQ ID NO: 545) |
| 10A9V3-30 | QVQLVESGGGVVQPGRSLRLSCAASGYPFSNFWITWVRQAPGKGLEWIGDI |
| | YPGSDNRNFNDSVKGRFTISRDNSKNTLYLQMNSLRAEDTAVYYCAREAYY |
| | TNPGFAYWGQGTLVTVSS (SEQ ID NO: 546) |
| 10A9V3-23 | EVQLLESGGGLVQPGGSLRLSCAASGYPFSNFWITWVRQAPGKGLEWIGDIY |
| | PGSDNRNFNDSVKGRFTISRDNSKNTLYLQMNSLRAEDTAVYYCAREAYYT |
| | NPGFAYWGQGTLVTVSS (SEQ ID NO: 547) |
| 10A9V4-59 | QVQLQESGPGLVKPSETLSLTCTVSGYPFSNFWITWIRQPPGKGLEWIGDIYP |
| | GSDNRNFNPSLKSRVTISVDTSKNQFSLKLSSVTAADTAVYYCAREAYYTNP |
| | GFAYWGQGTLVTVSS (SEQ ID NO: 548) |
| 10A9V3-15 | EVQLVESGGGLVKPGGSLRLSCAASGYPFSNFWITWVRQAPGKGLEWIGDIY |
| | PGSDNRNFNAPVKGRFTISRDDSKNTLYLQMNSLKTEDTAVYYCAREAYYT |
| | NPGFAYWGQGTLVTVSS (SEQ ID NO: 549) |
| 10A9V4-39 | QLQLQESGPGLVKPSETLSLTCTVSGYPFSNFWITWIRQPPGKGLEWIGDIYP |
| | GSDNRNFNPSLKSRVTISVDTSKNQFSLKLSSVTAADTAVYYCAREAYYTNP |
| | GFAYWGQGTLVTVSS (SEQ ID NO: 550) |
| Antibody 11A8 | Antibody 11A8 |
| 11A8V3-15 | EVQLVESGGGLVKPGGSLRLSCAASGFNFNTYAMNWVRQAPGKGLEWVAR |
| | IRSKSNNYATYYAAPVKGRFTISRDDSKNTLYLQMNSLKTEDTAVYYCVRH |
| | YSNYGWGFAYWGQGTLVTVSS (SEQ ID NO: 552) |
| 11A8V3-48 | EVQLVESGGGLVQPGGSLRLSCAASGFNFNTYAMNWVRQAPGKGLEWVAR |
| | IRSKSNNYATYYADSVKGRFTISRDNAKNSLYLQMNSLRAEDTAVYYCVRH |
| | YSNYGWGFAYWGQGTLVTVSS (SEQ ID NO: 553) |
| 11A8V3-23 | EVQLLESGGGLVQPGGSLRLSCAASGFNFNTYAMNWVRQAPGKGLEWVARI |
| | RSKSNNYATYYADSVKGRFTISRDNSKNTLYLQMNSLRAEDTAVYYCVRHY |
| | SNYGWGFAYWGQGTLVTVSS (SEQ ID NO: 554) |
| 11A8V3-30 | QVQLVESGGGVVQPGRSLRLSCAASGFNFNTYAMNWVRQAPGKGLEWVAR |
| | IRSKSNNYATYYADSVKGRFTISRDNSKNTLYLQMNSLRAEDTAVYYCVRH |
| | YSNYGWGFAYWGQGTLVTVSS (SEQ ID NO: 555) |
| 11A8V3-7 | EVQLVESGGGLVQPGGSLRLSCAASGFNFNTYAMNWVRQAPGKGLEWVAR |
| | IRSKSNNYATYYADSVKGRFTISRDNAKNSLYLQMNSLRAEDTAVYYCVRH |
| | YSNYGWGFAYWGQGTLVTVSS (SEQ ID NO: 553) |
| 11A8V1-69 | QVQLVQSGAEVKKPGSSVKVSCKASGFNFNTYAMNWVRQAPGQGLEWVAR |
| | IRSKSNNYATYYAQKFQGRVTITADESTSTAYMELSSLRSEDTAVYYCVRHY |
| | SNYGWGFAYWGQGTLVTVSS (SEQ ID NO: 556) |
| 11A8V1-46 | QVQLVQSGAEVKKPGASVKVSCKASGFNFNTYAMNWVRQAPGQGLEWVA |
| | RIRSKSNNYATYYAQKFQGRVTMTRDTSTSTVYMELSSLRSEDTAVYYCVR |
| | HYSNYGWGFAYWGQGTLVTVSS (SEQ ID NO: 557) |
| 11A8V5-51 | EVQLVQSGAEVKKPGESLKISCKGSGFNFNTYAMNWVRQMPGKGLEWVARI |
| | RSKSNNYATYYAPSFQGQVTISADKSISTAYLQWSSLKASDTAMYYCVRHYS |
| | NYGWGFAYWGQGTLVTVSS (SEQ ID NO: 558) |
| 11A8V4-59 | QVQLQESGPGLVKPSETLSLTCTVSGFNFNTYAMNWIRQPPGKGLEWVARIR |
| | SKSNNYATYYAPSLKSRVTISVDTSKNQFSLKLSSVTAADTAVYYCVRHYSN |
| | YGWGFAYWGQGTLVTVSS (SEQ ID NO: 559) |
| 11A8V4-39 | QLQLQESGPGLVKPSETLSLTCTVSGFNFNTYAMNWIRQPPGKGLEWVARIR |
| | SKSNNYATYYAPSLKSRVTISVDTSKNQFSLKLSSVTAADTAVYYCVRHYSN |
| | YGWGFAYWGQGTLVTVSS (SEQ ID NO: 560) |
| Antibody 12D9 | Antibody 12D9 |
| 12D9V1-46 | QVQLVQSGAEVKKPGASVKVSCKASGYTFSDYYIHWVRQAPGQGLEWIGYI |
| | YPNNGDNGYNQKFQGRVTMTRDTSTSTVYMELSSLRSEDTAVYYCARRGY |
| | YGGSYDYWGQGTLVTVSS (SEQ ID NO: 562) |
| 12D9V5-51 | EVQLVQSGAEVKKPGESLKISCKGSGYTFSDYYIHWVRQMPGKGLEWIGYIY |
| | PNNGDNGYNPSFQGQVTISADKSISTAYLQWSSLKASDTAMYYCARRGYYG |
| | GSYDYWGQGTLVTVSS (SEQ ID NO: 563) |
| 12D9V1-69 | QVQLVQSGAEVKKPGSSVKVSCKASGYTFSDYYIHWVRQAPGQGLEWIGYI |
| | YPNNGDNGYNQKFQGRVTITADESTSTAYMELSSLRSEDTAVYYCARRGYY |
| | GGSYDYWGQGTLVTVSS (SEQ ID NO: 564) |
| 12D9V3-48 | EVQLVESGGGLVQPGGSLRLSCAASGYTFSDYYIHWVRQAPGKGLEWIGYIY |
| | PNNGDNGYNDSVKGRFTISRDNAKNSLYLQMNSLRAEDTAVYYCARRGYY |
| | GGSYDYWGQGTLVTVSS (SEQ ID NO: 565) |
| 12D9V3-30 | QVQLVESGGGVVQPGRSLRLSCAASGYTFSDYYIHWVRQAPGKGLEWIGYI |
| | YPNNGDNGYNDSVKGRFTISRDNSKNTLYLQMNSLRAEDTAVYYCARRGY |
| | YGGSYDYWGQGTLVTVSS (SEQ ID NO: 566) |
| 12D9V3-23 | EVQLLESGGGLVQPGGSLRLSCAASGYTFSDYYIHWVRQAPGKGLEWIGYIY |
| | PNNGDNGYNDSVKGRFTISRDNSKNTLYLQMNSLRAEDTAVYYCARRGYY |
| | GGSYDYWGQGTLVTVSS (SEQ ID NO: 567) |
| 12D9V3-7 | EVQLVESGGGLVQPGGSLRLSCAASGYTFSDYYIHWVRQAPGKGLEWIGYIY |
| | PNNGDNGYNDSVKGRFTISRDNAKNSLYLQMNSLRAEDTAVYYCARRGYY |
| | GGSYDYWGQGTLVTVSS (SEQ ID NO: 565) |
| 12D9V4-59 | QVQLQESGPGLVKPSETLSLTCTVSGYTFSDYYIHWIRQPPGKGLEWIGYIYP |
| | NNGDNGYNPSLKSRVTISVDTSKNQFSLKLSSVTAADTAVYYCARRGYYGG |
| | SYDYWGQGTLVTVSS (SEQ ID NO: 568) |
| 12D9V3-15 | EVQLVESGGGLVKPGGSLRLSCAASGYTFSDYYIHWVRQAPGKGLEWIGYIY |
| | PNNGDNGYNAPVKGRFTISRDDSKNTLYLQMNSLKTEDTAVYYCARRGYY |
| | GGSYDYWGQGTLVTVSS (SEQ ID NO: 569) |
| 12D9V4-30-4 | QVQLQESGPGLVKPSQTLSLTCTVSGYTFSDYYIHWIRQPPGKGLEWIGYIYP |
| | NNGDNGYNPSLKSRVTISVDTSKNQFSLKLSSVTAADTAVYYCARRGYYGG |
| | SYDYWGQGTLVTVSS (SEQ ID NO: 570) |
| Antibody 12F9 | Antibody 12F9 |
| 12F9V3-15 | EVQLVESGGGLVKPGGSLRLSCAASGFRFNTYAMTWVRQAPGKGLEWEGVI |
| | RRKSSNFATLYAAPVKGRFTISRDDSKNTLYLQMNSLKTEDTAVYYCVRHK |
| | SNKYPFVYWGQGTLVTVSS (SEQ ID NO: 572) |
| 12F9V3-23 | EVQLLESGGGLVQPGGSLRLSCAASGFRFNTYAMTWVRQAPGKGLEWEGVI |
| | RRKSSNFATLYADSVKGRFTISRDNSKNTLYLQMNSLRAEDTAVYYCVRHK |
| | SNKYPFVYWGQGTLVTVSS (SEQ ID NO: 573) |
| 12F9V3-48 | EVQLVESGGGLVQPGGSLRLSCAASGFRFNTYAMTWVRQAPGKGLEWEGVI |
| | RRKSSNFATLYADSVKGRFTISRDNAKNSLYLQMNSLRAEDTAVYYCVRHK |
| | SNKYPFVYWGQGTLVTVSS (SEQ ID NO: 574) |
| 12F9V3-30 | QVQLVESGGGVVQPGRSLRLSCAASGFRFNTYAMTWVRQAPGKGLEWEGV |
| | IRRKSSNFATLYADSVKGRFTISRDNSKNTLYLQMNSLRAEDTAVYYCVRHK |
| | SNKYPFVYWGQGTLVTVSS (SEQ ID NO: 575) |
| 12F9V3-7 | EVQLVESGGGLVQPGGSLRLSCAASGFRFNTYAMTWVRQAPGKGLEWEGVI |
| | RRKSSNFATLYADSVKGRFTISRDNAKNSLYLQMNSLRAEDTAVYYCVRHK |
| | SNKYPFVYWGQGTLVTVSS (SEQ ID NO: 574) |
| 12F9V1-69 | QVQLVQSGAEVKKPGSSVKVSCKASGFRFNTYAMTWVRQAPGQGLEWEGV |
| | IRRKSSNFATLYAQKFQGRVTITADESTSTAYMELSSLRSEDTAVYYCVRHK |
| | SNKYPFVYWGQGTLVTVSS (SEQ ID NO: 576) |
| 12F9V1-46 | QVQLVQSGAEVKKPGASVKVSCKASGFRFNTYAMTWVRQAPGQGLEWEG |
| | VIRRKSSNFATLYAQKFQGRVTMTRDTSTSTVYMELSSLRSEDTAVYYCVRH |
| | KSNKYPFVYWGQGTLVTVSS (SEQ ID NO: 577) |
| 12F9V5-51 | EVQLVQSGAEVKKPGESLKISCKGSGFRFNTYAMTWVRQMPGKGLEWEGVI |
| | RRKSSNFATLYAPSFQGQVTISADKSISTAYLQWSSLKASDTAMYYCVRHKS |
| | NKYPFVYWGQGTLVTVSS (SEQ ID NO: 578) |
| 12F9V4-59 | QVQLQESGPGLVKPSETLSLTCTVSGFRFNTYAMTWIRQPPGKGLEWEGVIR |
| | RKSSNFATLYAPSLKSRVTISVDTSKNQFSLKLSSVTAADTAVYYCVRHKSN |
| | KYPFVYWGQGTLVTVSS (SEQ ID NO: 579) |
| 12F9V4-39 | QLQLQESGPGLVKPSETLSLTCTVSGFRFNTYAMTWIRQPPGKGLEWEGVIR |
| | RKSSNFATLYAPSLKSRVTISVDTSKNQFSLKLSSVTAADTAVYYCVRHKSN |
| | KYPFVYWGQGTLVTVSS (SEQ ID NO: 580) |
| Antibody 10C1 | Antibody 10C1 |
| 10C1V3-15 | EVQLVESGGGLVKPGGSLRLSCAASGFTFSDAWMDWVRQAPGKGLEWVAE |
| | IRNKINNHATYYAAPVKGRFTISRDDSKNTLYLQMNSLKTEDTAVYYCTSLY |
| | DGSYLRFAYWGQGTLVTVSS (SEQ ID NO: 647) |
| 10C1V3-7 | EVQLVESGGGLVQPGGSLRLSCAASGFTFSDAWMDWVRQAPGKGLEWVAE |
| | IRNKINNHATYYADSVKGRFTISRDNAKNSLYLQMNSLRAEDTAVYYCTSLY |
| | DGSYLRFAYWGQGTLVTVSS (SEQ ID NO: 648) |
| 10C1V3-23 | EVQLLESGGGLVQPGGSLRLSCAASGFTFSDAWMDWVRQAPGKGLEWVAEI |
| | RNKINNHATYYADSVKGRFTISRDNSKNTLYLQMNSLRAEDTAVYYCTSLY |
| | DGSYLRFAYWGQGTLVTVSS (SEQ ID NO: 649) |
| 10C1V3-30 | QVQLVESGGGVVQPGRSLRLSCAASGFTFSDAWMDWVRQAPGKGLEWVAE |
| | IRNKINNHATYYADSVKGRFTISRDNSKNTLYLQMNSLRAEDTAVYYCTSLY |
| | DGSYLRFAYWGQGTLVTVSS (SEQ ID NO: 650) |
| 10C1V3-48 | EVQLVESGGGLVQPGGSLRLSCAASGFTFSDAWMDWVRQAPGKGLEWVAE |
| | IRNKINNHATYYADSVKGRFTISRDNAKNSLYLQMNSLRAEDTAVYYCTSLY |
| | DGSYLRFAYWGQGTLVTVSS (SEQ ID NO: 651) |
| 10C1V1-69 | QVQLVQSGAEVKKPGSSVKVSCKASGFTFSDAWMDWVRQAPGQGLEWVA |
| | EIRNKINNHATYYAQKFQGRVTITADESTSTAYMELSSLRSEDTAVYYCTSL |
| | YDGSYLRFAYWGQGTLVTVSS (SEQ ID NO: 652) |
| 10C1V1-46 | QVQLVQSGAEVKKPGASVKVSCKASGFTFSDAWMDWVRQAPGQGLEWVA |
| | EIRNKINNHATYYAQKFQGRVTMTRDTSTSTVYMELSSLRSEDTAVYYCTSL |
| | YDGSYLRFAYWGQGTLVTVSS (SEQ ID NO: 653) |
| 10C1V5-51 | EVQLVQSGAEVKKPGESLKISCKGSGFTFSDAWMDWVRQMPGKGLEWVAE |
| | IRNKINNHATYYAPSFQGQVTISADKSISTAYLQWSSLKASDTAMYYCTSLY |
| | DGSYLRFAYWGQGTLVTVSS (SEQ ID NO: 654) |
| 10C1V4-59 | QVQLQESGPGLVKPSETLSLTCTVSGFTFSDAWMDWIRQPPGKGLEWVAEIR |
| | NKINNHATYYAPSLKSRVTISVDTSKNQFSLKLSSVTAADTAVYYCTSLYDGS |
| | YLRFAYWGQGTLVTVSS (SEQ ID NO: 655) |
| 10C1V4-30-4 | QVQLQESGPGLVKPSQTLSLTCTVSGFTFSDAWMDWIRQPPGKGLEWVAEIR |
| | NKINNHATYYAPSLKSRVTISVDTSKNQFSLKLSSVTAADTAVYYCTSLYDG |
| | SYLRFAYWGQGTLVTVSS (SEQ ID NO: 656) |
| Antibody 7E9 | Antibody 7E9 |
| 7E9V1-46 | QVQLVQSGAEVKKPGASVKVSCKASGYTFTEYTMHWVRQAPGQGLEWIGG |
| | INPNNGGTSYKQKFQGRVTMTRDTSTSTVYMELSSLRSEDTAVYYCARGGS |
| | HYYAMDYWGQGTLVTVSS (SEQ ID NO: 658) |
| 7E9V1-69 | QVQLVQSGAEVKKPGSSVKVSCKASGYTFTEYTMHWVRQAPGQGLEWIGG |
| | INPNNGGTSYKQKFQGRVTITADESTSTAYMELSSLRSEDTAVYYCARGGSH |
| | YYAMDYWGQGTLVTVSS (SEQ ID NO: 659) |
| 7E9V5-51 | EVQLVQSGAEVKKPGESLKISCKGSGYTFTEYTMHWVRQMPGKGLEWIGGI |
| | NPNNGGTSYKPSFQGQVTISADKSISTAYLQWSSLKASDTAMYYCARGGSH |
| | YYAMDYWGQGTLVTVSS (SEQ ID NO: 660) |
| 7E9V3-23 | EVQLLESGGGLVQPGGSLRLSCAASGYTFTEYTMHWVRQAPGKGLEWIGGI |
| | NPNNGGTSYKDSVKGRFTISRDNSKNTLYLQMNSLRAEDTAVYYCARGGSH |
| | YYAMDYWGQGTLVTVSS (SEQ ID NO: 661) |
| 7E9V3-30 | QVQLVESGGGVVQPGRSLRLSCAASGYTFTEYTMHWVRQAPGKGLEWIGGI |
| | NPNNGGTSYKDSVKGRFTISRDNSKNTLYLQMNSLRAEDTAVYYCARGGSH |
| | YYAMDYWGQGTLVTVSS (SEQ ID NO: 662) |
| 7E9V3-48 | EVQLVESGGGLVQPGGSLRLSCAASGYTFTEYTMHWVRQAPGKGLEWIGGI |
| | NPNNGGTSYKDSVKGRFTISRDNAKNSLYLQMNSLRAEDTAVYYCARGGSH |
| | YYAMDYWGQGTLVTVSS (SEQ ID NO: 663) |
| 7E9V3-7 | EVQLVESGGGLVQPGGSLRLSCAASGYTFTEYTMHWVRQAPGKGLEWIGGI |
| | NPNNGGTSYKDSVKGRFTISRDNAKNSLYLQMNSLRAEDTAVYYCARGGSH |
| | YYAMDYWGQGTLVTVSS (SEQ ID NO: 664) |
| 7E9V4-59 | QVQLQESGPGLVKPSETLSLTCTVSGYTFTEYTMHWIRQPPGKGLEWIGGIN |
| | PNNGGTSYKPSLKSRVTISVDTSKNQFSLKLSSVTAADTAVYYCARGGSHYY |
| | AMDYWGQGTLVTVSS (SEQ ID NO: 665) |
| 7E9V3-15 | EVQLVESGGGLVKPGGSLRLSCAASGYTFTEYTMHWVRQAPGKGLEWIGGI |
| | NPNNGGTSYKAPVKGRFTISRDDSKNTLYLQMNSLKFEDTAVYYCARGGSH |
| | YYAMDYWGQGTLVTVSS (SEQ ID NO: 666) |
| 7E9V4-39 | QLQLQESGPGLVKPSETLSLTCTVSGYTFTEYTMHWIRQPPGKGLEWIGGIN |
| | PNNGGTSYKPSLKSRVTISVDTSKNQFSLKLSSVTAADTAVYYCARGGSHYY |
| | AMDYWGQGTLVTVSS (SEQ ID NO: 667) |
| Antibody 8C3 | Antibody 8C3 |
| 8C3V1-46 | QVQLVQSGAEVKKPGASVKVSCKASGYSFTGYYMHWVRQAPGQGLEWIGR |
| | VNPNNGGTSYNQKFQGRVTMTRDTSTSTVYMELSSLRSEDTAVYYC |
| | VLTGGYFDYWGQGTLVTVSS (SEQ ID NO: 669) |
| 8C3V5-51 | EVQLVQSGAEVKKPGESLKISCKGSGYSFTGYYMHWVRQMPGKGLEWIGR |
| | VNPNNGGTSYNPSFQGQVTISADKSISTAYLQWSSLKASDTAMYYCVLTGG |
| | YFDYWGQGTLVTVSS (SEQ ID NO: 670) |
| 8C3V3-23 | EVQLLESGGGLVQPGGSLRLSCAASGYSFTGYYMHWVRQAPGKGLEWIGR |
| | VNPNNGGTSYNDSVKGRFTISRDNSKNTLYLQMNSLRAEDTAVYYCVLTGG |
| | YFDYWGQGTLVTVSS (SEQ ID NO: 671) |
| 8C3V1-69 | QVQLVQSGAEVKKPGSSVKVSCKASGYSFTGYYMHWVRQAPGQGLEWIGR |
| | VNPNNGGTSYNQKFQGRVTITADESTSTAYMELSSLRSEDTAVYYCVLTGG |
| | YFDYWGQGTLVTVSS (SEQ ID NO: 672) |
| 8C3V3-30 | QVQLVESGGGVVQPGRSLRLSCAASGYSFTGYYMHWVRQAPGKGLEWIGR |
| | VNPNNGGTSYNDSVKGRFTISRDNSKNTLYLQMNSLRAEDTAVYYCVLTGG |
| | YFDYWGQGTLVTVSS (SEQ ID NO: 673) |
| 8C3V3-48 | EVQLVESGGGLVQPGGSLRLSCAASGYSFTGYYMHWVRQAPGKGLEWIGR |
| | VNPNNGGTSYNDSVKGRFTISRDNAKNSLYLQMNSLRAEDTAVYYCVLTGG |
| | YFDYWGQGTLVTVSS (SEQ ID NO: 674) |
| 8C3V3-7 | EVQLVESGGGLVQPGGSLRLSCAASGYSFTGYYMHWVRQAPGKGLEWIGR |
| | VNPNNGGTSYNDSVKGRFTISRDNAKNSLYLQMNSLRAEDTAVYYCVLTGG |
| | YFDYWGQGTLVTVSS (SEQ ID NO: 675) |
| 8C3V4-59 | QVQLQESGPGLVKPSETLSLTCTVSGYSFTGYYMHWIRQPPGKGLEWIGRVN |
| | PNNGGTSYNPSLKSRVTISVDTSKNQFSLKLSSVTAADTAVYYCVLTGGYFD |
| | YWGQGTLVTVSS (SEQ ID NO: 676) |
| 8C3V3-15 | EVQLVESGGGLVKPGGSLRLSCAASGYSFTGYYMHWVRQAPGKGLEWIGR |
| | VNPNNGGTSYNAPVKGRFTISRDDSKNTLYLQMNSLKTEDTAVYYCVLTGG |
| | YFDYWGQGTLVTVSS (SEQ ID NO: 677) |
| 8C3V4-39 | QLQLQESGPGLVKPSETLSLTCTVSGYSFTGYYMHWIRQPPGKGLEWIGRVN |
| | PNNGGTSYNPSLKSRVTISVDTSKNQFSLKLSSVTAADTAVYYCVLTGGYFD |
| | YWGQGTLVTVSS (SEQ ID NO: 678) |

Humanization of Antibody 9F5

The heavy chain variable region (VH) and light chain variable region (VL) sequences of murine anti-TREM2 antibody 9F5 (T2-9F5.1) were used as input to the IgBLAST program on the NCBI website (Ye, J. et al. Nucleic Acids Research 41:W34-W40 (2013)). IgBLAST takes a murine VH or VL sequence and compares it to a library of known human germline sequences. The databases used were IMGT human VH genes (F+ORF, 273 germline sequences) and IMGT human VLkappa genes (F+ORF, 74 germline sequences). For the 2F5 antibody VL, human germline IGKV2-29 (allele 2) was chosen as a good acceptor sequence and human light chain IGKJ2(allele 1) joining region (J gene) was chosen from human joining region sequences compiled at IMGT® the international ImMunoGeneTics information System® (FIG. 4C). For the 2F5 antibody VH, human germline IGHV1-46(allele 1) was chosen as a good acceptor sequence and the human heavy chain IGHJ4(allele 1) joining region (J gene) was chosen from human joining region sequences compiled at IMGT® the international ImMunoGeneTics information System® (FIG. 4D). Complementarity determing regions (CDRs) for the antibody VL and VH were defined according to the AbM definition (AbM antibody modeling software).

Alteration of human germline framework (i.e., non-CDR residues in the VH and VL) positions to corresponding parental murine sequences may be required to optimize binding of the humanized antibody. Potential changes for each humanized sequence are noted in FIGS. 4C and 4D.

FIGS. 4C and 4D show sequences of humanized versions of anti-TREM2 antibody 9F5. In the in CDR-L1 of theVL domain of antibody 9F5, Asp30c-Gly30d has a high potential for deamidation followed by isoaspartate formation (FIG. 4C).

Post-translational modification at this site may affect binding of the antibody to its target. The 9F5 may be humanized and then in a final step the NG may be altered, for example, to QG, and tested to determine if binding is maintained (FIG. 4C). In CDR-L2, Asn53 has a low potential for deamidation based on sequence and conformation but may show a low level of deamidation and may be changed to reduce deamidation risk (FIG. 4C). Variant VL sequences based on the above are listed in Table 7A.

In the VH domain of antibody 9F5, there are two Asn (Asn58 and Asn 98) that have low potential for deamidation based on sequence and conformation, and may show a low level of these post-translational modifications (FIG. 4D). In additioan, Asn58 and Asn 98 may be changed to reduce deamidation risk (FIG. 4D). In CDR-H1, Trp33 is likely to be solvent-exposed and have potential for oxidation, especially under stress conditions (FIG. 4D). Accordingly, Trp33 may be changed to reduce oxidation risk. In CDR-H2, Asp54-Gly55 has a medium potential for isoaspartate formation (FIG. 4D). Thus, post-translational modification at Asp54-Gly55 may affect binding of the antibody to its target. The 9F5 may be humanized and then in a final step the DG, may be altered, for example, toEG or other amino acids, and tested to determine if binding is maintained. Variant VH sequences based on the above are listed in Table 7B.

### Example 2: Epitope Mapping of TREM2 Antibodies

TREM2 antibodies were tested for their ability to bind 15-mer or 25-mer peptides spanning the entire human TREM2 (SEQ ID NO: 1) and mouse TREM2 (SEQ ID NO: 2). Additionally, the epitopes of the anti-TREM2 antibodies 9F5 (MAb), T21-9 (Fab), T22 (Fab), and T45-10 (Fab) were mapped by shotgun mutatagenesis.

Methodology

Linear 15-mer peptides were synthesized based on the sequence of human TREM2 (SEQ ID NO: 1), with a 14 residue overlap. In addition, linear 25-mer peptides were synthesized based on sequence of human TREM2 (SEQ ID NO: 1) or mouse TREM2 (SEQ ID NO: 2) with a single residue shift. The binding of TREM2 antibodies to each of the synthesized peptides was tested in an ELISA based method. In this assay, the peptide arrays were incubated with primary antibody solution (overnight at 4° C.). After washing, the peptide arrays were incubated with a 1/1000 dilution of an antibody peroxidase conjugate (SBA, cat. nr. 2010-05) for one hour at 25° C. After washing, the peroxidase substrate 2,2′-azino-di-3-ethylbenzthiazoline sulfonate (ABTS) and 2 μl/ml of 3% H₂O₂ were added. After one hour, the color development was measured. The color development was quantified with a charge coupled device (CCD) camera and an image processing system.

Alternatively, to reconstruct epitopes of the target molecule, libraries of looped and combinatorial peptides were synthesized. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer).

Synthesis of structural mimics was done using Pepscan's proprietary Chemically Linked Peptides on Scaffolds (CLIPS) technology. CLIPS technology allows to structure peptides into single loops and double-loops. CLIPS templates are coupled to cysteine residues. The side-chains of multiple cysteines in the peptides are coupled to one or two CLIPS templates. For example, a 0.5 mM solution of the mP2 CLIPS (2,6-bis(bromomethyl)pyridine) is dissolved in ammonium bicarbonate (20 mM, pH 7.8)/acetonitrile (1:3(v/v)). This solution is added onto the peptide arrays. The CLIPS template will bind to side-chains of two cysteines as present in the solid-phase bound peptides of the peptide-arrays (455 wells plate with 3 μl wells). The peptide arrays are gently shaken in the solution for 30 to 60 minutes while completely covered in solution. Finally, the peptide arrays are washed extensively with excess of H₂O and sonicated in disrupt-buffer containing 1% SDS/0.1% β-mercaptoethanol in PBS (pH 7.2) at 70° C. for 30 minutes, followed by sonication in H₂O for another 45 minutes. The T3 CLIPS (2,4,6-tris(bromomethyl)pyridine) carrying peptides were made in a similar way but now with three cysteines.

Looped Peptides:

constrained peptides of length 17. Positions 2-16 are 15-mers derived from the target sequence. Native Cys residues are protected by acetamidomethyl group (ACM). Positions 1 and 17 are Cys that are linked by mP2 CLIPS moieties. Combinatorial peptides (discontinuous mimics): constrained peptides of length 33. Positions 2-16 and 18-32 are 15-mer peptides derived from the target sequence with native Cys residues protected by ACM. Positions 1, 17 and 33 are Cys that are linked by T3 CLIPS moieties.

The binding of antibody to each of the synthesized peptides is tested in a PEPSCAN-based ELISA. The peptide arrays are incubated with test antibody solution composed of the experimentally optimized concentration of the test antibody and blocking solution (for example 4% horse serum, 5% ovalbumin (w/v) in PBS/1% Tween80). The peptide arrays are incubated with the test antibody solution overnight at 4° C. After extensive washing with washing buffer (1×PBS, 0.05% Tween80), the peptide arrays are incubated with a 1/1000 dilution of an appropriate antibody peroxidase conjugate for one hour at 25° C. After washing with the washing buffer, the peroxidase substrate 2,2′-azino-di-3-ethylbenzthiazoline sulfonate (ABTS) and 2 μl/ml of 3% H₂O₂ are added. After one hour, the color development is measured. The color development is quantified with a charge coupled device (CCD)—camera and an image processing system.

Alternatively a mass spectrometry method is used to identify conformational epitopes. In order to determine the key residues of conformational epitopes on the TREM2 protein that anti-TREM2 antibodies bind to with high resolution, antibody/antigen complexes are incubated with deuterated cross-linkers and subjected to multi-enzymatic proteolytic cleavage. After enrichment of the cross-linked peptides, the samples are analyzed by high resolution mass spectrometry (nLC-Orbitrap MS) and the data generated is analyzed using XQuest software. Specifically, TREM2 ECD/antibody complexes are generated by mixing equimolar solutions of TREM2 antigen and antibody (4 μM in 5 μl each). One μ1 of the mixture obtained is mixed with 1 μl of a matrix composed of a re-crystallized sinapinic acid matrix (10 mg/ml) in acetonitrile/water (1:1, v/v), TFA 0.1% (K200 MALDI Kit). After mixing, 1 μl of each sample is spotted on a MALDI plate (SCOUT 384). After crystallization at room temperature, the plate is introduced in aMALDI mass spectrometer and analyzed immediately. The analysis is repeated in triplicate. Peaks representing monomeric antibody, the antigen, and antibody and antigen/antibody complexes are detected at the predicted molecular weights.

It is then determined whether the epitope in conformational binding competes with unstructured C1q peptides generated by proteolysis. Specifically, to determine if TREM2 ECD/antibody complexes can compete with linear peptides, the TREM2 ECD antigen is digested with immobilized pepsin. 25 μl of the antigen with a concentration of 10 μM are mixed with immobilized pepsin 5 μM and incubate at room temperature for 30 minutes. After the incubation time, the sample are centrifuged and the supernatant is pipetted. The completion of the proteolysis is controlled by High-Mass MALDI mass spectrometry in linear mode. The pepsin proteolysis is optimized in order to obtain a large amount of peptide in the 1000-3500 Da range. Next, 5 μl of the antigen peptides generated by proteolysis are mixed with 5 μl of antibodies (8 μM) and incubated at 37° C. for 6 hours. After incubation of the antibodies with the TREM2 antigen peptides, 5 μl of the mixture is mixed with 5 μl of the intact TREM2 antigen (4 μM) so the final mix contains 2 μM/2 μM/2.5 μM of TREM2/antibody/TREM2 antigen peptides. The MALDI ToF MS analysis is performed using CovalX's HM3 interaction module with a standard nitrogen laser and focusing on different mass ranges from 0 to 2000 kDa. For the analysis, the following parameters are applied for the mass spectrometer: Linear and Positive mode; Ion Source 1: 20 kV; Ion Source 2: 17 kV; Pulse Ion Extraction: 400 ns; for HM3: Gain Voltage: 3.14 kV; Gain Voltage: 3.14 kV; Acceleration Voltage: 20 kV. To calibrate the instrument, an external calibration with clusters of Insulin, BSA and IgG is being applied. For each sample, 3 spots are analyzed (300 laser shots per spots). Presented spectrum corresponds to the sum of 300 laser shots. The MS data are analyzed using the Complex Tracker analysis software version 2.0 (CovalX Inc). To identify the conformational epitopes for TREM2 binding to antibodies, using chemical cross-linking, High-Mass MALDI mass spectrometry and nLCOrbitrap mass spectrometry the interaction interface between the antigen and antibodies the following procedure is followed. 5 μl of the sample antigen (concentration 4 μM) is mixed with 5 μl of the sample antibody (Concentration 4 μM) in order to obtain an antibody/antigen mix with final concentration 2 μM/2 μM. The mixture is incubated at 37° C. for 180 minutes. In a first step, 1 mg of DiSuccinimidylSuberate H12 (DSS-H12) cross-linker is mixed with 1 mg of DiSuccinimidylSuberate D12 (DSS-D12) cross-linker. The 2 mg prepared were mixed with 1 ml of DMF in order to obtain a 2 mg/ml solution of DSS H12/D12. 10 μl of the antibody/antigen mix prepared previously were mixed with 1 μl of the solution of cross-linker d0/d12 prepared (2 mg/ml). The solution is incubated 180 minutes at room temperature in order to achieve the cross-linking reaction.

In order to facilitate the proteolysis, it is necessary to reduce the disulfide bonds present in the protein. The cross-linked samples are mixed with 20 μl of ammonium bicarbonate (25 mM, pH 8.3). After mixing 2.5 μl of DTT (500 mM) is added to the solution. The mixture is then incubated 1 hour at 55° C. After incubation, 2.5 μl of iodioacetamide (1 M) is added before 1 hour of incubation at room temperature in a dark room. After incubation, the solution is diluted 1/5 by adding 120 μl of the buffer used for the proteolysis. 145 μl of the reduced/alkyled cross-linked sample is mixed with 2 μl of trypsin (Sigma, T6567). The proteolytic mixture is incubated overnight at 37° C. For a-chymotrypsin proteolysis, the buffer of proteolysis is Tris-HCL 100 mM, CaCl2 10 mM, pH7.8. The 145 μl of the reduced/alkyled cross-linked complex is mixed with 2 μl of α-chymotrypsin 200 μM and incubated overnight at 30° C. For this analysis, an nLC in combination with Orbitrap mass spectrometry is used. The cross-linker peptides are analyzed using Xquest version 2.0 and stavrox software. The peptides and cross-linked amino acids are then identified.

Results

The TREM2 binding region was determined for 26 anti-TREM2 antibodies. The binding regions within human and/or mouse TREM2 are listed in Tables 8A and 8B.

TABLE 8A
TREM2 antibody binding region to human TREM2

| Antibody | Human TREM2 binding region | Amino acid region of SEQ ID NO: 1 |
|---|---|---|
| 5A2 | ³⁵SCPYDSMKHWGRRKA⁴⁹ and | 35-49 and 140-150 |
| 7D1 | ¹⁴⁰DLWFPGESESF¹⁵⁰ | |
| 8E10 | ³⁹DSMKHWGRRKA⁴⁹ and | 39-49 and 63-77 |
| | ⁶³VVSTHNLWLLSFLRR⁷⁷ | |
| 2H8 | ⁵¹CRQLGEKGPCQ⁶¹ | 51-61 |
| 18E4 | ⁵⁵GEKGPCQR⁶²,¹⁰⁴DAGLYQ¹⁰⁹, | 55-62, 104-109, and 148-158 |
| | and ¹⁴⁸ESFEDAHVEHS¹⁵⁸ | |
| 44A8 | ⁵⁵GEKGPCQR⁶², ¹⁰⁴DAGLYQ¹⁰⁹, and | 55-62, 104-109, and 160-166 |
| | ¹⁶⁰SRSLLEG¹⁶⁶ | |
| 18D8 | ⁵⁵GEKGPCQRVVS⁶⁵ and | 55-65 and 124-134 |
| | ¹²⁴VLVEVLADPLD¹³⁴ | |
| 49E12 | ⁶³VVSTHNLWLLS⁷³ and | 63-73 and 156-170 |
| 11D7 | ¹⁵⁶EHSISRSLLEGEIPF¹⁷⁰ | |
| 6H6 | ¹¹⁷EADTLRKVLVEVLADPL¹³³ | 117-133 |
| 8C3 | ¹³⁷DAGDLWFPGE¹⁴⁶ | 137-146 |
| 7E9 | | |
| 12F9 | | |
| 9G1 | | |
| 9G3 | | |
| 11A8 | | |
| 7B3 | | |
| 3B10 | ¹³⁹GDLWFPGES¹⁴⁷ | 139-147 |
| 8F8 | | |
| 11H5 | ¹³⁹GDLWFPGESES¹⁴⁹ | 139-149 |
| 6G12 | | |
| 10A9 | | |
| 10C1 | | |
| 3A2 | | |
| 2F6 | | |
| 1B4 | ¹⁴⁰DLWFPGE¹⁴⁶ | 140-146 |
| 29F6 | | |
| 7B11 | | |
| 40D5 | | |
| 45D6 | | |
| 29F7 | | |
| 21D10 | | |
| 3A7 | ¹⁴²WFPGESESFED¹⁵² | 142-152 |
| 7E5 | | |
| 6H2 | ¹⁴⁶ESESFEDAH¹⁵⁴ | 146-154 |
| 12G6 | ¹⁴⁹SFEDAHVEH¹⁵⁷ | 149-157 |
| 9F5 | | |
| 2A7 | ¹⁵⁴HVEHSISR¹⁶¹ | 154-161 |
| 9B4 | | |

TABLE 8
TREM2 antibody binding region to mouse TREM2

| Anti- | MouseTREM2 | Amino acid region of |
|---|---|---|
| body | binding region | SEQ ID NO: 2 |
| 2H8 | ⁵¹CRQLGEEGPCQ⁶¹ | 51-61 |
| 3B10 | | |
| 8F8 | ¹³⁹GDLWVPEES¹⁴⁷ | 139-147 |
| 2F6 | | |
| 3A7 | ¹⁴²WVPEESSSFEG¹⁵² | 142-152 |
| 7E5 | | |

As indicated in Table 8A, antibodies 5A2 and 7D1 showed robust binding exclusively for peptides within two extracellular domains (including the IgV domain) of human TREM2. As indicated in Table 8A, the peptides recognized by antibodies 5A2 and 7D1 correspond to amino acid residues 35-49 and 140-150 of SEQ ID NO: 1 and have the amino acid sequences of: SCPYDSMKHWGRRKA and DLWFPGESESF.

As indicated in Table 8A, antibody 8E10 showed robust binding exclusively for peptides within the extracellular IgV domain of human TREM2. As indicated in Table 8A, the peptides recognized by antibody 8E10 correspond to amino acid residues 39-49 and 63-77 of SEQ ID NO: 1 and have the amino acid sequences of: DSMKHWGRRKA and VVSTHNLWLLSFLRR.

As indicated in Tables 8A and 8B, antibody 2H8 showed robust binding exclusively for a peptide within the extracellular IgV domain of human and mouse TREM2. As indicated in Table 8A, the human TREM2 peptide recognized by antibody 2H8 corresponds to amino acid residues 51-61 of SEQ ID NO: 1 and has the amino acid sequence of: CRQLGEKGPCQ. As indicated in Table 8B, the mouse TREM2 peptide recognized by antibody 2H8 corresponds to amino acid residues 51-61 of SEQ ID NO: 2 and has the amino acid sequence of: CRQLGEEGPCQ.

As indicated in Table 8A, antibody 18E4 showed robust binding exclusively for peptides within the extracellular domain of human TREM2. As indicated in Table 8A, the peptides recognized by antibody 18E4 correspond to amino acid residues 55-62, 104-109, and 148-158 of SEQ ID NO: 1 and have the amino acid sequences of: GEKGPCQR, DAGLYQ, and ESFEDAHVEHS. Moreover, it was determined that a key sequence involved in binding corresponds to amino acid residues 151-155 of SEQ ID NO: 1 and has the amino acid sequence of: EDAHV.

As indicated in Table 8A, antibody 44A8 showed robust binding exclusively for peptides within the extracellular domain of human TREM2. As indicated in Table 8A, the peptides recognized by antibody 44A8 correspond to amino acid residues 55-62, 104-109, and 160-166 of SEQ ID NO: 1 and have the amino acid sequences of: GEKGPCQR, DAGLYQ, and SRSLLEG. Moreover, it was determined that a key sequence involved in binding corresponds to amino acid residues 162-165 of SEQ ID NO: 1 and has the amino acid sequence of: SLLE.

As indicated in Table 8A, antibody 18D8 showed robust binding exclusively for peptide within the extracellular domain of human TREM2. As indicated in Table 8A, the peptide recognized by antibody 18D8 correspond to amino acid residues 55-65 and 124-134 of SEQ ID NO: 1 and have the amino acid sequences of: GEKGPCQRVVS and VLVEVLADPLD.

As indicated in Table 8A, antibodies 49E12 and 11D7 showed robust binding exclusively for peptides within the extracellular domain of human TREM2. As indicated in Table 8A, the peptides recognized by antibodies 49E12 and 11D7 correspond to amino acid residues 63-73 and 156-170 of SEQ ID NO: 1 and have the amino acid sequences of: VVSTHNLWLLS and EHSISRSLLEGEIPF.

As indicated in Table 8A, antibody 6H6 showed robust binding exclusively for a peptide within the extracellular domain of human TREM2. As indicated in Table 8A, the peptide recognized by antibody 6H6 corresponds to amino acid residues 117-133 of SEQ ID NO: 1 and has the amino acid sequence of: EADTLRKVLVEVLADPL.

As indicated in Table 8A, antibodies 8C3, 7E9, 12F9, 9G1, 9G3, 11A8, and 7B3 showed robust binding exclusively for a peptide within the extracellular domain of human TREM2. As indicated in Table 8A, the peptide recognized by antibodies 8C3, 7E9, 12F9, 9G1, 9G3, 11A8, and 7B3 corresponds to amino acid residues 137-146 of SEQ ID NO: 1 and has the amino acid sequence of: DAGDLWFPGE.

As indicated in Tables 8A and 8B, antibodies 3B10 and 8F8 showed robust binding exclusively for a peptide within the extracellular domain of human and mouse TREM2. As indicated in Table 8A, the human TREM2 peptide recognized by antibodies 3B10 and 8F8 corresponds to amino acid residues 139-147 of SEQ ID NO: 1 and has the amino acid sequence of: GDLWFPGES. As indicated in Table 8B, the mouse TREM2 peptide recognized by antibodies 3B10 and 8F8 corresponds to amino acid residues 139-147 of SEQ ID NO: 2 and has the amino acid sequence of: GDLWVPEES. Moreover, the human and mouse peptide recognized by antibodies 3B10 and 8F8 has the amino sequence of: GDLW[F/V]P[G/E]ES (SEQ ID NO: 884).

As indicated in Table 8A, antibodies 11H5, 6G12, 10A9, 10C1, and 3A2 showed robust binding exclusively for a peptide within the extracellular domain of human TREM2. As indicated in Table 8A, the peptide recognized by antibodies 11H5, 6G12, 10A9, 10C1, and 3A2 corresponds to amino acid residues 139-149 of SEQ ID NO: 1 and has the amino acid sequence of: GDLWFPGESES.

As indicated in Tables 8A and 8B, antibody 2F6 showed robust binding exclusively for a peptide within the extracellular domain of human and mouse TREM2. As indicated in Table 8A, the human TREM2 peptide recognized by antibody 2F6 corresponds to amino acid residues 139-149 of SEQ ID NO: 1 and has the amino acid sequence of: GDLWFPGESES. As indicated in Table 8B, the mouse TREM2 peptide recognized by antibody 2F6 corresponds to amino acid residues 139-147 of SEQ ID NO: 2 and has the amino acid sequence of: GDLWVPEES.

As indicated in Table 8A, antibodies 1B4, 29F6, 7B11, 40D5, 45D6, 29F7, and 21D10 showed robust binding exclusively for a peptide within the extracellular domain of human and mouse TREM2. As indicated in Table 8A, the peptide recognized by antibodies 1B4, 29F6, 7B11, 40D5, 45D6, 29F7, and 21D10 corresponds to amino acid residues 140-146 of SEQ ID NO: 1 and has the amino acid sequence of: DLWFPGE. Moreover, it was determined that a key sequence involved in binding corresponds to amino acid residues 140-143 of SEQ ID NO: 1 and has the amino acid sequence of: DLWF.

As indicated in Tables 8A and 8B, antibodies 3A7 and 7E5 showed robust binding exclusively for a peptide within the extracellular domain of human and mouse TREM2. As indicated in Table 8A, the human TREM2 peptide recognized by antibodies 3A7 and 7E5 corresponds to amino acid residues 142-152 of SEQ ID NO: 1 and has the amino acid sequence of: WFPGESESFED. As indicated in Table 8B, the mouse TREM2 peptide recognized by antibodies 3A7 and 7E5 corresponds to amino acid residues 142-152 of SEQ ID NO: 1 and has the amino acid sequence of: WVPEESSSFEG.

As indicated in Table 8A, antibody 6H2 showed robust binding exclusively for a peptide within the extracellular domain of human TREM2. As indicated in Table 8A, the peptide recognized by antibody 6H2 corresponds to amino acid residues 146-154 of SEQ ID NO: 1 and has the amino acid sequence of: ESESFEDAH. Moreover, it was determined that key amino acid residues involved in binding correspond to amino acid residues S149 and F150 of SEQ ID NO: 1.

As indicated in Table 8A, antibodies 12G6 and 9F5 showed robust binding exclusively for a peptide within the extracellular domain of human TREM2. As indicated in Table 8A, the peptide recognized by antibodies 12G6 and 9F5 corresponds to amino acid residues 149-157 of SEQ ID NO: 1 and has the amino acid sequence of: SFEDAHVEH.

As indicated in Table 8A, antibodies 2A7 and 9B4 showed robust binding exclusively for a peptide within the extracellular domain of human TREM2. As indicated in Table 8A, the peptide recognized by antibodies 2A7 and 9B4 corresponds to amino acid residues 154-161 of SEQ ID NO: 1 and has the amino acid sequence of: HVEHSISR.

Shotgun Mutagenesis Epitope Mapping

Shotgun mutagenesis epitope mapping of anti-TREM2 antibodies (9F5 (MAb), T21-9 (Fab), T22 (Fab), and T45-10 (Fab)) was performed using an alanine-scanning library for the TREM2 protein. A TREM2-Dap12 expression construct encoding a full length hTREM2-Dap12 chimera was subjected to high-throughput alanine scanning mutagenesis (outlined in Davidson and Doranz, 2014 Immunology 143, 13-20) to generate a comprehensive mutation library. Each of residues 19 to 174 of TREM2 was mutated, representing the TREM2 extracellular domain, most to alanine, while alanine codons were mutated to serine. In total, 154 TREM2 mutant expression constructs were generated, sequences confirmed, and arrayed into a 384-well plate, one mutant per well.

The TREM2 mutation library clones, arrayed in a 384-well microplate, were transfected individually into HEK-293T cells and allowed to express for 22 hours. Cells were then incubated with the indicated MAb or Fabs diluted in 10% normal goat serum (NGS) (Sigma-Aldrich, St. Louis, Mo.). Prior to library screening, primary MAb and Fab concentrations were determined using an independent immunofluorescence titration curve against cells expressing wild-type TREM2 to ensure that signals were within the linear range of detection. The MAb was detected using 3.75 μg/ml AlexaFluor488-conjugated secondary antibody (Jackson ImmunoResearch Laboratories, Westgrove, Pa., Cat. #115-545-003) in 10% NGS, while the Fabs were detected using 7.50 μg/ml AlexaFluor488-conjugated secondary antibody (Jackson ImmunoResearch Laboratories, Westgrove, Pa., Cat. #109-546-006) in 10% NGS. Cells were washed twice with PBS −/− and resuspended in Cellstripper (Cellgro, Manassas, Va.) with 0.1% BSA (Sigma-Aldrich, St. Louis, Mo.). Mean cellular fluorescence was detected using the Intellicyt High Throughput Flow Cytometer (HTFC, Intellicyt, Albuquerque, N. Mex.). MAb and Fab reactivities against each mutant clone were calculated relative to wild-type TREM2 protein reactivity by subtracting the signal from mock-transfected controls, and normalizing to the signal from wild-type TREM2-transfected controls.

Mutated residues within critical clones were identified as critical to the MAb or Fab epitope if they did not support reactivity of the test MAb or Fab but did support reactivity of other test antibodies. This counter-screen strategy facilitated the exclusion of TREM2 mutants that were locally misfolded or that had an expression defect.

FIG. 4E depicts the mean binding reactivities and ranges for all critical residues identified in the screens. The range was the difference between the duplicate experimental binding values. Primary critical residues were identified as residues where mutations were negative for test antibody binding (<30% of binding to WT for Fab T21-9; <20% of binding to WT for MAb 9F5 and Fabs T22 and T45-10) but positive for other test antibodies (>70% WT).

The amino acid residues critical for antibody binding are listed in Table 8C.

TABLE 8C
Residues involved in anti-TREM2 antibody MAb and Fab binding

| Antibody | Critical TREM2 residues | Secondary TREM2 residues |
|---|---|---|
| 9F5 MAb | E₁₅₁; D₁₅₂; H₁₅₄; and E₁₅₆ | |
| T21-9 Fab | K₄₂ and H₁₁₄ | |
| T22 Fab | K₄₂; G₄₅; and H₁₁₄ | H₄₃; W₄₄; and E₁₁₇ |
| T45-10 Fab | R₇₇ | H₆₇ and T₈₈ |

As indicated in Table 8C, the critical TREM2 residues involved in binding by MAb 9F5 corresponded to amino acid residues E₁₅₁, D₁₅₂, H₁₅₄, and E₁₅₆ of SEQ ID NO: 1. The critical TREM2 residues involved in binding by Fab T21-9 corresponded to amino acid residues K₄₂ and H₁₁₄ of SEQ ID NO: 1. The critical TREM2 residues involved in binding by Fab T22 corresponded to amino acid residues K₄₂, G₄₅, and H₁₁₄ of SEQ ID NO: 1; and the secondary residues involved in binding by Fab T22 correspond to amino acid residues H₄₃, W₄₄, and E₁₁₇. The critical TREM2 residue involved in binding by Fab T45-10 corresponded to amino acid residue R₇₇ of SEQ ID NO: 1; and the secondary residues involved in binding by Fab T45-10 correspond to amino acid residues H₆₇ and T₈₈. The secondary residues involved in binding by Fab T22 and Fab T45-10 contribute to a lesser extent to the overall energy of binding.

### Example 3: TREM2 Antibodies Induce Syk Phosphorylation

Spleen tyrosine kinase (Syk) is an intracellular signaling molecule that functions downstream of TREM2 by phosphorylating several substrates, thereby facilitating the formation of a signaling complex leading to cellular activation and inflammatory processes. The ability of agonist TREM2 antibodies to induce Syk activation was determined by culturing mouse macrophages and measuring the phosphorylation state of Syk protein in cell extracts.

Bone marrow-derived macrophages (BMDM) from wild-type (WT) mice, from TREM2 knockout (KO) mice, and from mice that lack expression of functional Fc receptor common gamma chain gene (FcgR KO; REF: Takai T 1994. Cell 76(3):519-29) were starved for 4 hours in 1% serum RPMI and then removed from tissue culture dishes with PBS-EDTA, washed with PBS, and counted. The cells were coated with full-length TREM2 antibodies 2F6, 11H5, 2H8, 1H7, 3A7, 3B10, 7F8, and 7E5, or with control antibodies (10A9 or msIgG1 isotype control) for 15 minutes on ice. After washing with cold PBS, cells were incubated at 37° C. for the indicated period of time in the presence of goat anti-human IgG. After stimulation, cells were lysed with lysis buffer (1% v/v NP-40%, 50 Mm Tris-HCl (pH 8.0), 150 mM NaCl, 1 mM EDTA, 1.5 mM MgCl₂, 10% glycerol, plus protease and phosphatase inhibitors) followed by centrifugation at 16,000 g for 10 mM at 4° C. to remove insoluble materials. Lysates were then immunoprecipitated with anti-Syk antibody (N-19 for BMDM or 4D10 for human DCs, Santa Cruz Biotechnology). Precipitated proteins were fractionated by SDS-PAGE, transferred to PVDF membranes and probed with anti-phosphotyrosine antibody (4G10, Millipore). To confirm that all substrates were adequately immunoprecipitated, immunoblots were reprobed with anti-Syk antibody (Abcam, for BMDM) or anti-Syk (Novus Biological, for human DCs). Visualization was performed with the enhanced chemiluminescence (ECL) system (GE healthcare), as described (e.g., Peng et al., (2010) Sci Signal., 3(122): ra38).

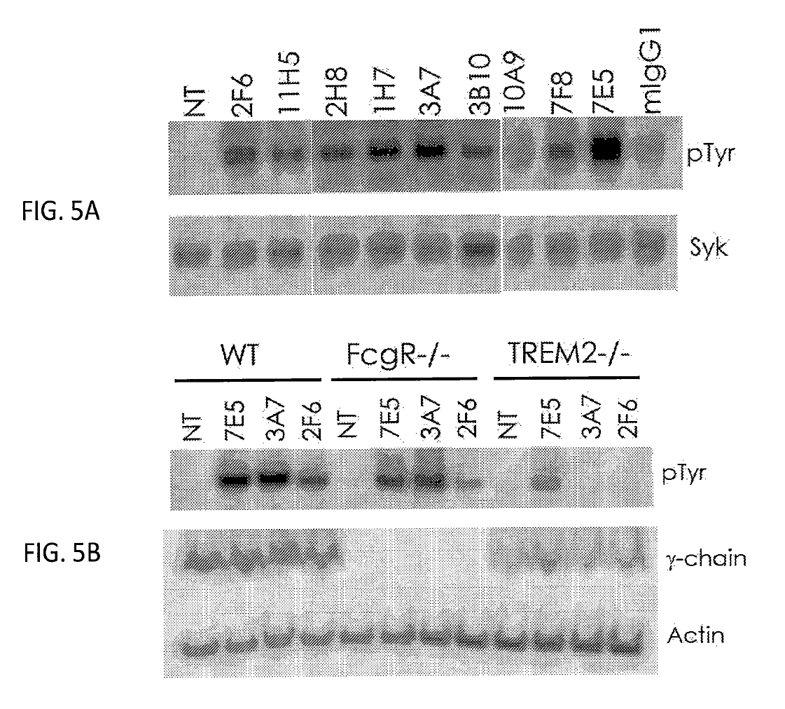

As shown in FIG. 5A, TREM2 antibodies 2F6, 11H5, 2H8, 1H7, 3A7, 3B10, 7F8, and 7E5 induced TREM2-mediated Syk phosphorylation in BMDMs. Syk phosphorylation induced by antibodies 7E5, 3A7, and 2F6 is TREM2-specific, as Syk phosphorylation was not induced when TREM2 KO BMDM were used as a control (FIG. 5B). Syk phosphorylation induced by antibodies 7E5 and 3A7 does not require Fc receptor common gamma chain (FcgR), as Syk phosphorylation was not induced when FcgR KO BMDM were used as a control (FIG. 5B). Control antibodies 10A9 and msIgG1 did not induce a significant amount of Syk phosphorylation. Based on these results, TREM2 antibodies 2F6, 11H5, 2H8, 1H7, 3A7, 3B10, 7F8, and 7E5 function as agonist antibodies that induce Syk phosphorylation in macrophages.

### Example 4: TREM2 Antibodies Induce Syk Phosphorylation when Clustered by Adjacent Cells that Expresses Fc Gamma Receptors

Activation of spleen tyrosine kinase (Syk) is facilitated by crosslinking two or more TREM2 receptors with antibodies, thereby facilitating the formation of a signaling complex leading to cellular activation and inflammatory processes. In vivo cross-linking is mediated by adjacent cells that express high affinity Fc receptors (FcR), such as B cells and other leukocytes (White AL Cancer Immunol Immunother (2013) 62:941-948; Wilson N S 2011, Cancer Cell 19, 101-113; Bartholomaeus P J Immunol 2014; 192:2091-2098).

The ability of Fc receptors to induce activation of Syk through antibody clustering was determined by culturing mouse macrophages in the presence of cells expressing Fc receptors and measuring the phosphorylation state of Syk protein in cell extracts. Bone marrow-derived macrophages (BMDM) from wild-type (WT) mice and from TREM2 knockout (KO) mice were starved for 4 hours in 1% serum RPMI and then removed from tissue culture dishes with PBS-EDTA, washed with PBS, and counted. The cells were coated with full-length TREM2 antibodies 2F6, 11H5, 2H8, 1H7, 3A7, 3B10, 7F8, and 7E5, or control antibodies (10A9 or msIgG1 isotype control) for 15 minutes on ice. After washing with cold PBS, cells were incubated for 5 minutes at 37° C. with glutaraldehyde-fixed cells that express Fc receptors and that were previously prepared as follows. Briefly, Fc receptors expressing cells were either B cells isolated from mouse spleens using MACS microbeads (CD19⁺ B-cell isolation kit Miltenyi Biotec) according to the manufacturer's protocol or alternatively the P815 cell line that overexpresses FcR2b and FcR3. 2×10⁶ cells/ml cells were fixed with 0.05% glutaraldehyde for 1 minute at room temperature, the reaction was stopped with 1 μM Glycine and cells were then washed extensively with PBS. After stimulation, cells were lysed with lysis buffer (1% v/v NP-40%, 50 Mm Tris-HCl (pH 8.0), 150 mM NaCl, 1 mM EDTA, 1.5 mM MgCl₂, 10% glycerol, plus protease and phosphatase inhibitors) followed by centrifugation at 16,000 g for 10 mM at 4° C. to remove insoluble materials. Lysates were then immunoprecipitated with anti-Syk antibody (N-19 for BMDM or 4D10 for human DCs, Santa Cruz Biotechnology). Precipitated proteins were fractionated by SDS-PAGE, transferred to PVDF membranes and probed with anti-phosphotyrosine antibody (4G10, Millipore). To confirm that all substrates were adequately immunoprecipitated, immunoblots were reprobed with anti-Syk antibody (Abcam, for BMDM) or anti-Syk (Novus Biological, for human DCs). Visualization was performed with the enhanced chemiluminescence (ECL) system (GE healthcare), as described (e.g., Peng et al., (2010) Sci Signal., 3(122): ra38).

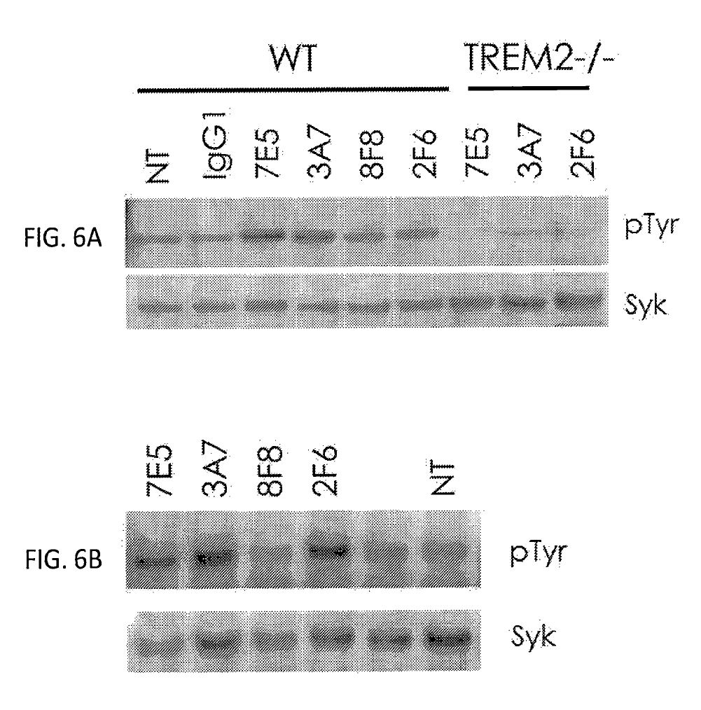

TREM2 antibodies 3A7 and 7E5 induced TREM-2 mediated Syk phosphorylation in BMDMs upon clustering with P815 cells, while antibodies 8F8 and 2F6 did not activate Syk phosphorylation, as compared to the isotype control msIgG1 (FIG. 6A). Syk phosphorylation induced by antibodies 3A7 and 7E5 is TREM2-specific, as Syk phosphorylation was not induced when TREM2 KO BMDM were used as a control (FIG. 6A). TREM2 antibodies 2F6, 3A7, and 7E5 induced TREM2-mediated Syk phosphorylation in BMDMs upon clustering with primary splenic B cells, while antibody 8F8 did not activate Syk phosphorylation, as compared to the isotype control msIgG1 (FIG. 6B). Based on these results, TREM2 antibodies 7E5, 3A7, and 2F6 are agonist antibodies that induce Syk phosphorylation in primary macrophages when clustered by adjacent cell that expresses Fc gamma receptors.

### Example 5: TREM2 Antibodies Induce DAP12 Phosphorylation In Vitro and In Vivo

DAP12 Phosphorylation in Mouse Macrophages

TREM2 signals through DAP12, leading downstream to activation of PI3K and other intracellular signals. The ability of TREM2 antibodies to induce DAP12 activation was determined by culturing mouse macrophages and measuring the phosphorylation state of DAP12 protein in cell extracts. Before stimulation with antibodies, mouse wild-type (WT) bone marrow-derived macrophages (BMDM) and TREM2 knockout (KO) BMDM were starved for 4 h in 1% serum RPMI. 15×10⁶ cells were incubated in ice for 15 mM with full-length TREM2 antibodies or control antibodies. Cells were washed and incubated at 37° C. for the indicated period of time in the presence of goat anti-human IgG. After stimulation, cells were lysed with lysis buffer (1% v/v n-Dodecyl-β-D-maltoside, 50 Mm Tris-HCl (pH 8.0), 150 mM NaCl, 1 mM EDTA, 1.5 mM MgCl₂, 10% glycerol, plus protease and phosphatase inhibitors), followed by centrifugation at 16,000 g for 10 mM at 4° C. to remove insoluble materials. Cell lysate was immunoprecipitated with a second TREM2 antibody (R&D Systems). Precipitated proteins were fractionated by SDS-PAGE, transferred to PVDF membranes, and probed with anti-phosphotyrosine Ab (4G10, Millipore). The membrane was stripped and reprobed with anti-DAP12 antibody (Cells Signaling, D7G1X). Each cell lysate used for TREM2 immunoprecipitations contained an equal amount of proteins, as indicated by a control antibody (anti-Actin, Santa Cruz).

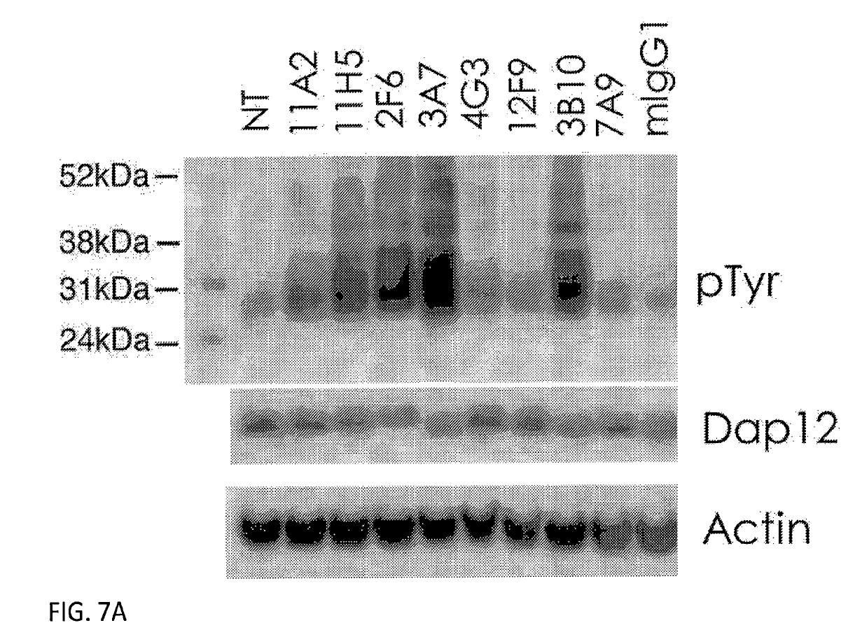

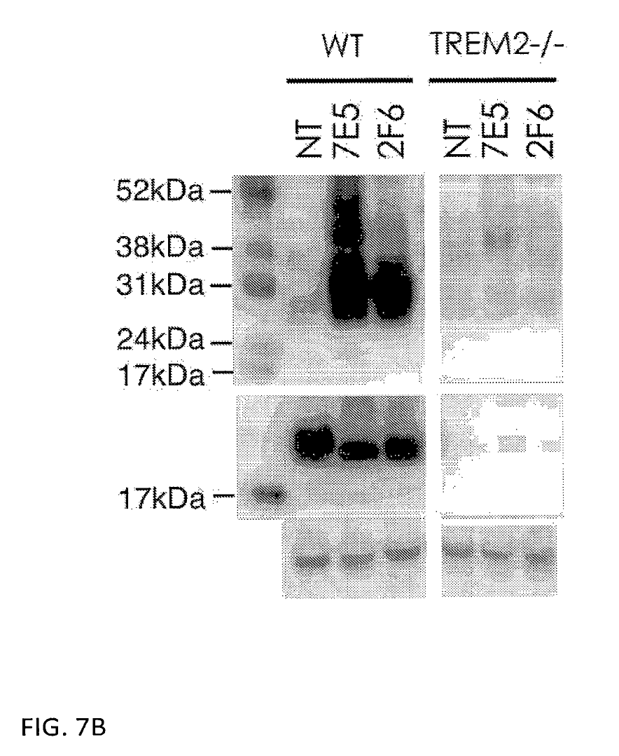

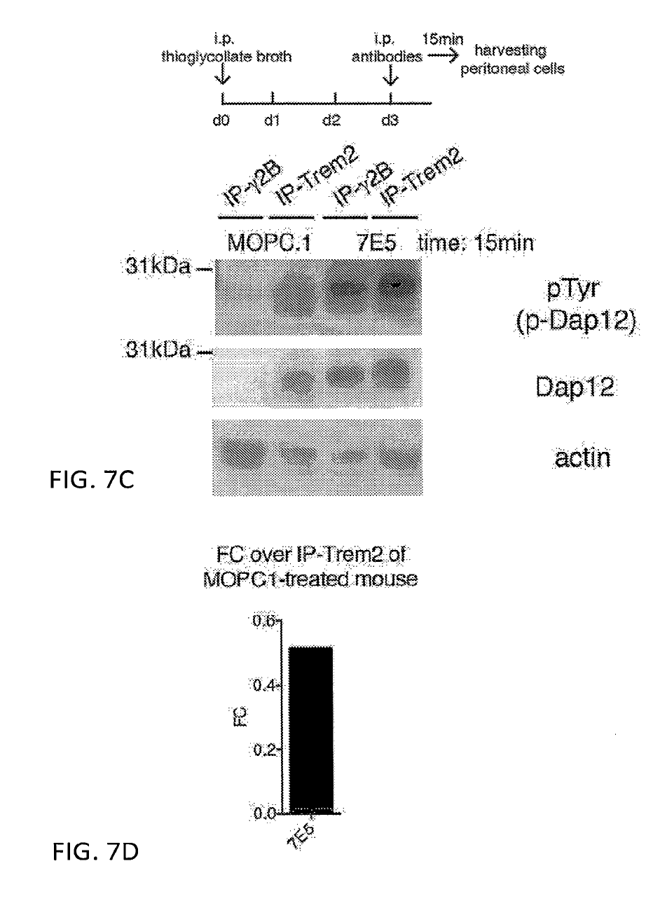

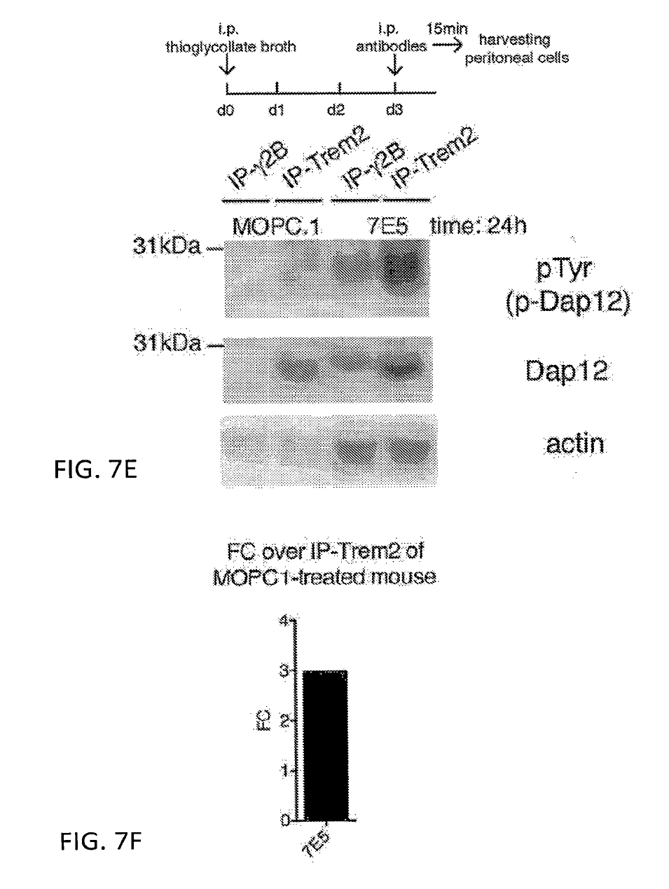

As shown in FIGS. 7A and 7B, DAP12 co-precipitated with TREM2 and was phosphorylated in WT macrophages incubated with TREM2 antibodies 11H5, 2F6, 3A7, 7E5, and 3B10, but not with antibodies 11A2, 4G3, 12F9, and 7A9, or the isotype control msIgG1 (FIGS. 7A and 7B). As a control, no DAP12 phosphorylation was observed in TREM2 KO (TREM2^{-/-}) macrophages incubated with antibodies 2F6 or 7E5 (FIG. 7B). These results demonstrate that TREM2 antibodies 2F6 and 7E5 are agonist antibodies that induce phosphorylation of TREM2-associated DAP12 in a TREM2-specific manner, as DAP12 phosphorylation is absent in TREM2-deficient BMDM.

In Vivo DAP12 Phosphorylation

The ability of TREM2 antibodies to induce DAP12 activation was determined in vivo on Brewer's Thioglycollate-induced peritoneal macrophages and measuring the phosphorylation state of DAP12 protein in cell extracts. C57B16 mice were injected intraperitoneally (i.p.) with 3 ml of 3% Brewer's Thioglycollate at day 0. At day 3, mice were injected intraperitoneally (i.p.) with isotype control antibody (CTR antibody) mIgG1 (clone MOPC-21, Bioxcell) or with anti-TREM2 antibody 7E5 for 15 minutes (FIGS. 7C and 7D) or for 24 hours (FIGS. 7E and 7F). Peritoneal cavity (PEC) cells were harvested by peritoneal lavage using 4 mL saline solution and washed with PBS. Cells were then lysed with lysis buffer (1% v/v n-Dodecyl-β-D-maltoside), 50 Mm Tris-HCl (pH 8.0), 150 mM NaCl, 1 mM EDTA, 1.5 mM MgCl₂, 10% glycerol, plus protease and phosphatase inhibitors), followed by centrifugation at 16,000 g for 10 mM at 4° C. to remove insoluble materials. Lysates recovered from each mouse sample were split and immunoprecipitated with a second TREM2 antibody (R&D Systems) or with isotype control Rat IgG2b antibody directly conjugated with beads. Precipitated proteins were fractionated by SDS-PAGE, transferred to PVDF membranes, and probed with anti-phosphotyrosine Ab (4G10, Millipore). The membrane was stripped and reprobed with anti-DAP12 antibody (Cells Signaling, D7G1X). Each cell lysate used for TREM2 immunoprecipitations was also probed with a control antibody (anti-Actin, Santa Cruz). The peritoneal fluid includes cells that express high levels of TREM2, as well as other cell types (e.g., eosinophils), and the number of cells harvested may vary. Therefore, some lysates recovered after immunoprecipitation were loaded on a gel and blotted with anti-actin.

This blot gives an indication of the total amount of cells (TREM2⁺ cells and TREM2⁻ cell) that were lysed. The graphs shown in FIGS. 7D and 7F indicate the fold change (FC) of TREM2-associated DAP12 phosphorylation upon in vivo stimulation with anti-TREM2 antibody 7E5 stimulation over the CTR antibody. Phosphorylated TREM2-associated DAP12 was normalized based on the amount of DAP12-associated TREM2.

It was previously shown that in vitro cross-linking (via cells expressing FcR) of anti-TREM2 antibody 7E5 induces clustering of TREM2 and induces TREM2-associated DAP12 phosphorylation. The results in FIG. 7C-7F indicate that DAP12 co-precipitated with TREM2 and was phosphorylated in peritoneal cells from mice treated with antibody 7E5, but not with the isotype control antibody (msIgG1). The results demonstrate that TREM2 antibody 7E5 is an agonist antibody that induces phosphorylation of TREM2-associated DAP12 in a TREM-2-specific manner, as DAP12 phosphorylation is absent in mice treated with control antibody.

### Example 6: Plate-Bound TREM2 Antibodies Induce TREM2-Dependent NFAT Promoter

The ability of plate-bound full-length anti-TREM2 antibodies to activate mouse or human TREM2-dependent genes was evaluated using a luciferase reporter gene under the control of an NFAT (nuclear factor of activated T-cells) promoter. The cell line BW5147.G.1.4 (ATCC® TIB48TM), derived from mouse thymus lymphoma T lymphocytes, was infected with mouse TREM2 and DAP12, and with Cignal Lenti NFAT-Luciferase virus (Qiagen). Alternatively the BW5147.G.1.4 cell line was infected with a human TREM2/DAP12 fusion protein, and with Cignal Lenti NFAT-Luciferase virus (Qiagen). As a positive control for signaling, PMA (0.05 ug/ml) and ionomycin (0.25 uM) were added together. Anti-TREM2 and isotype control antibodies were dissolved in PBS, plated on tissue culture plates at a concentration of 10 ug/ml and incubated overnight at 4° C. to allow the antibodies to absorb to the plate. After washing of the plates, cells were plated on the plate-bound antibodies and incubated for 6 hours. Luciferase activity was measured by adding OneGlo Reagent (Promega) to each well and incubating 3 min at room temperature on a plate shaker. Luciferase signal was measured using a BioTek plate reader. The cells display tonic TREM2-dependent signaling due to either the presence of an endogenous ligand or to spontaneous receptor aggregation, which leads to TREM2 signaling in the absence of additional stimulation.

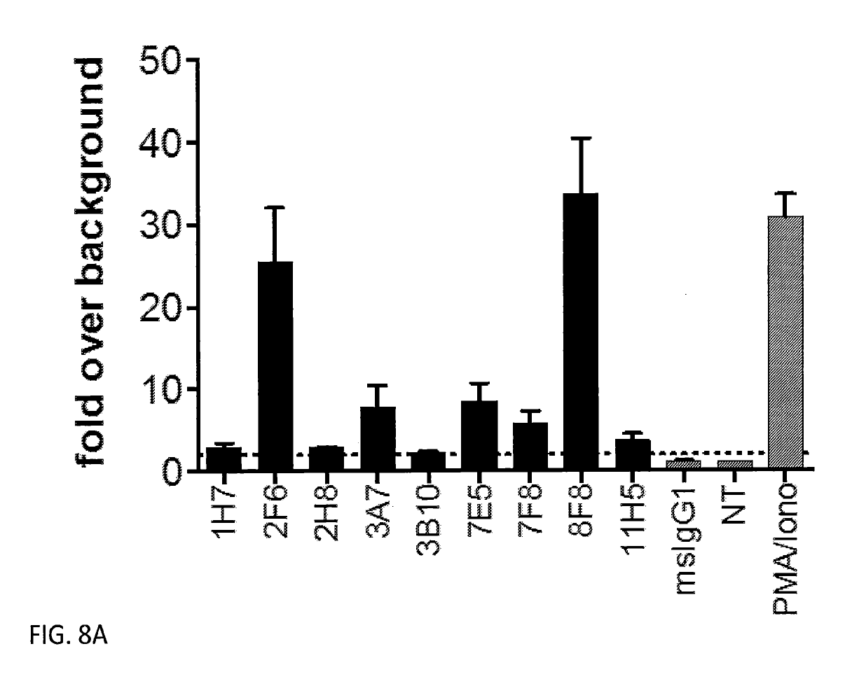

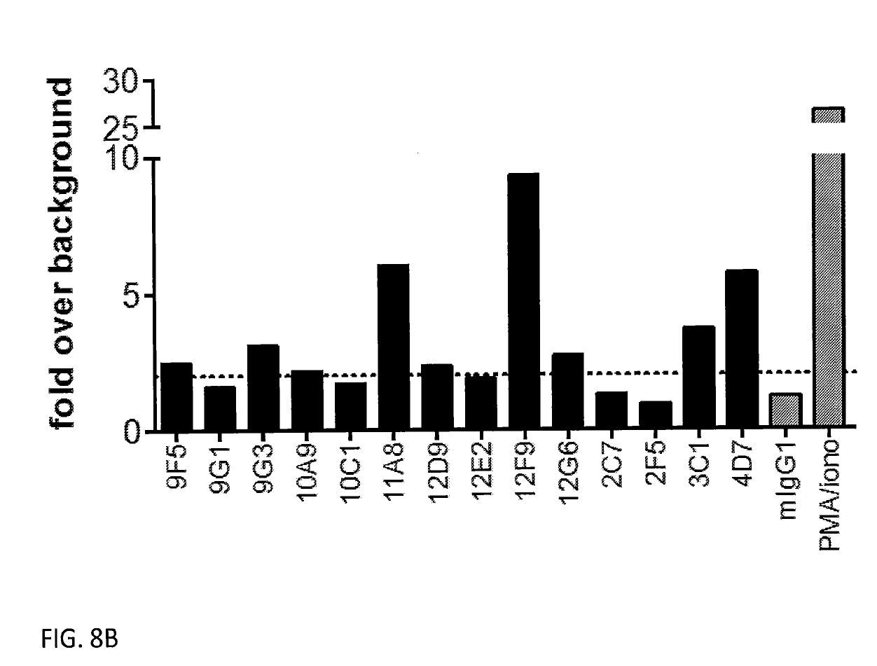

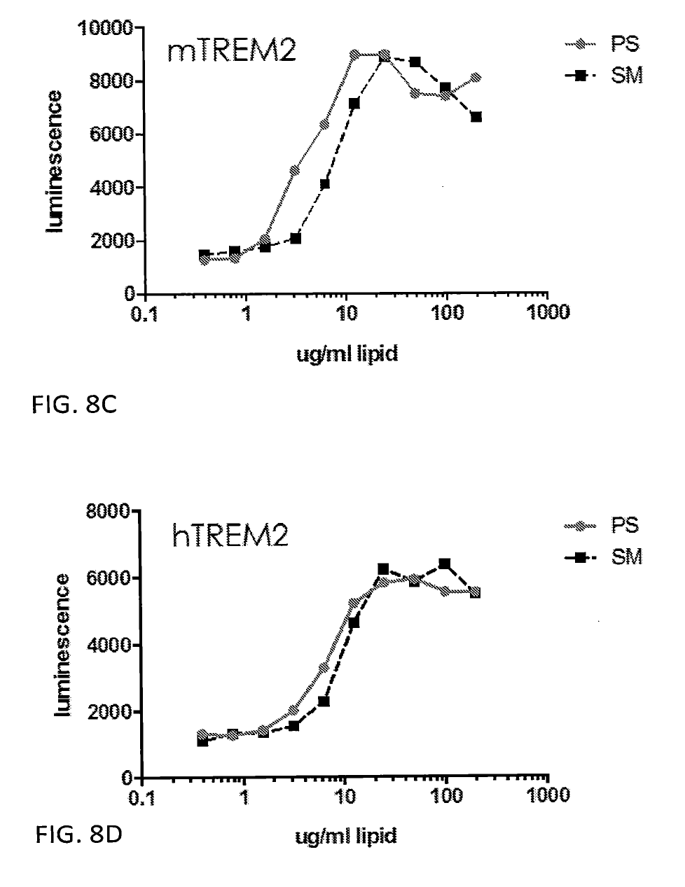

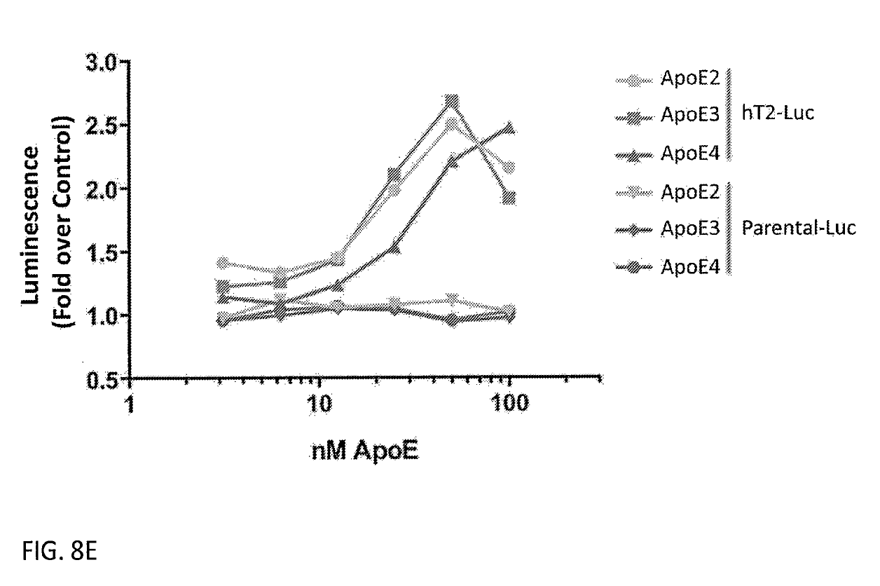

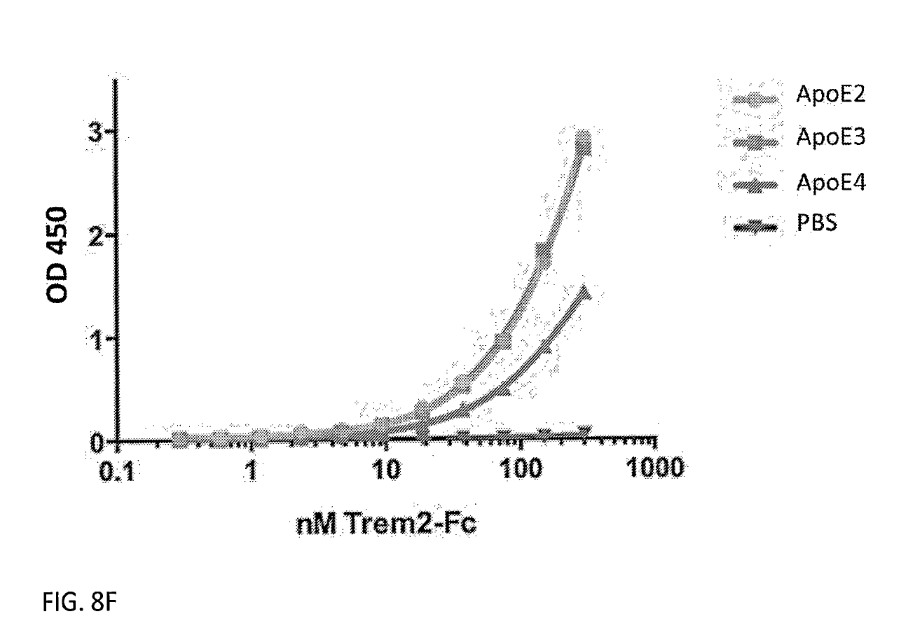

As shown in FIG. 8A, anti-TREM2 antibodies 2F6, 3A7, 7E5, 7F8, 8F8, and 11H5 increased luciferase activity in cells expressing mouse TREM2, as compared to the isotype control (msIgG1), indicating that the antibodies were able to induce TREM2-dependent gene transcription. As shown in FIG. 8B, anti-TREM2 antibodies 9F5, 9G3, 11A8, 12D9, 12F9, 12G6, 3C1, and 4D7 increased luciferase activity in cells expressing human TREM2, as compared to the isotype control (msIgG1), indicating that the antibodies were able to induce TREM2-dependent gene transcription. The dotted lines in FIGS. 8A and 8B indicate the levels of TREM2 activity without stimulation. FIGS. 8C and 8D show that plate-bound phosphatidylserine (PS) and sphingomyelin (SM) also induce NFAT promoter signaling in cells that express mouse TREM2 (FIG. 8C) and in cells that express human TREM2 (FIG. 8D). It is believed that PS and SM are natural ligands of TREM2. Thus, the results in FIG. 8A-8D indicate that agonist anti-TREM2 antibodies can mimic a natural ligand of TREM2.

### Example 7: TREM2 Ligands Induce TREM2-Dependent NFAT Promoter

The ability of natural TREM2 ligands to activate mouse or human TREM2-dependent genes was evaluated using the cell based luciferase reporter system described in Example 6. Plates were coated overnight with increasing concentration of phosphatidyl serine (PS), Sphingomyelin (SM) and human APOE variants APOE2, APOE3, and APOE4. After washing of the plates, cells were plated and incubated for 6 hours at 37° C. Luciferase activity was then measured by adding OneGlo Reagent (Promega), as described in Example 6. As previously describe, PS, SM, and the APOE variants provide activation of TREM2-dependent signaling.

Additionally, the ability of APO variants APOE2, APOE3, and APOE4 to bind recombinant human TREM2 protein was evaluated by ELISA. 2 μg/ml of ApoE protein was coated on high binding ELISA plates overnight at 4° C. On day 2, the plates were washed three times with 0.05% Tween in 1×PBS. Plates were then blocked with 3% milk at room temperature for 1 hour. The plates were incubated with 20 nM of TREM2-Fc protein. Then plates were washed 3 times at room temperature and incubated with secondary antibody goat anti-human Fc HRP for 1 hour. Plates were washed again three times at room temperature and 100 μL of TMB was added to each well. After the reaction had come to completion, 50 μL of 2N sulfuric acid was added per well to stop the reaction. Plates were read with a plate reader at 630 and 450 nm.

FIG. 8E shows that plate-bound APOE2, APOE3, and APOE4 also induce NFAT promoter activity in cells that express human TREM2. It is believed that the different APOE isoforms are natural ligand of TREM2. FIG. 8F shows that different APOE alleles (APOE2, APOE3 and APOE4) bind to recombinant TREM2 protein in an ELISA assay. Thus, the results indicate that agonistic anti-TREM2 antibodies can mimic a natural ligand of TREM2.

### Example 8: Soluble TREM2 Antibodies Induce TREM2-Dependent Genes

The ability of soluble full-length anti-TREM2 antibodies to activate mouse or human TREM2-dependent genes was evaluated using a luciferase reporter gene under the control of an NFAT (nuclear factor of activated T-cells) promoter. The cell line BW5147.G.1.4 (ATCC® TIB48TM), derived from mouse thymus lymphoma T lymphocytes, was infected with mouse TREM2 and DAP12, and with Cignal Lenti NFAT-Luciferase virus (Qiagen). Alternatively the BW5147.G.1.4 cell line was infected with a human TREM2/DAP12 fusion protein, and with Cignal Lenti NFAT-Luciferase virus (Qiagen). As a positive control for signaling, PMA (0.05 ug/ml) and ionomycin (0.25 uM) were added together. Cells were incubated together with soluble anti-TREM2 and isotype control antibodies for 6 hours and luciferase activity was measured by adding OneGlo Reagent (Promega) to each well and incubating 3 min at room temperature on a plate shaker. Luciferase signal was measured using a BioTek plate reader. The cells display tonic TREM2-dependent signaling due to either the presence of an endogenous ligand or to spontaneous receptor aggregation, which leads to TREM2 signaling.

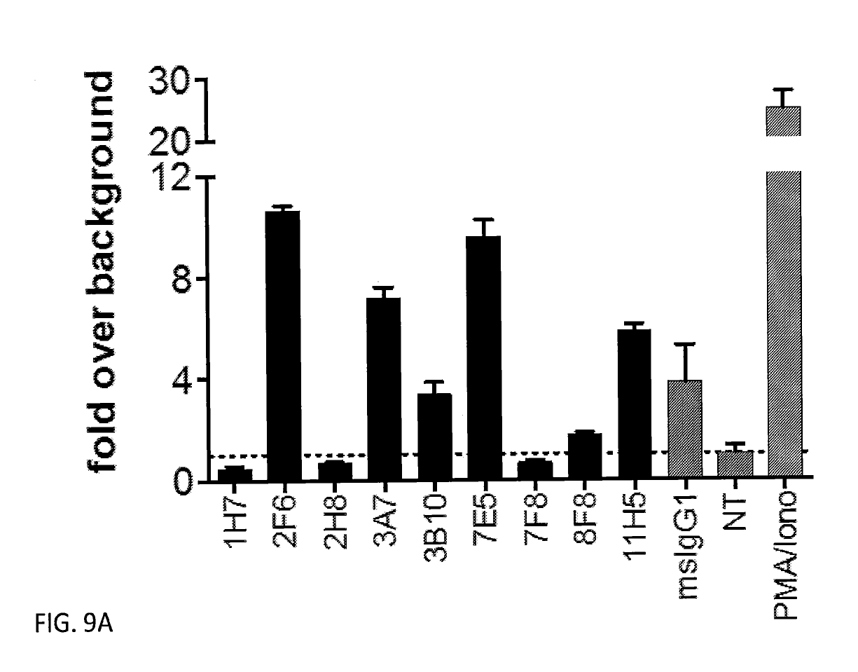

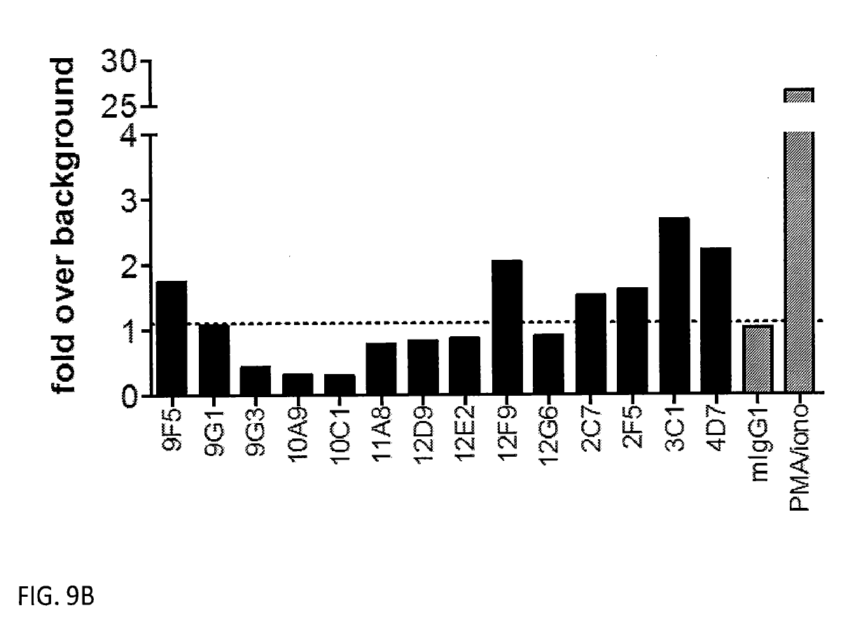

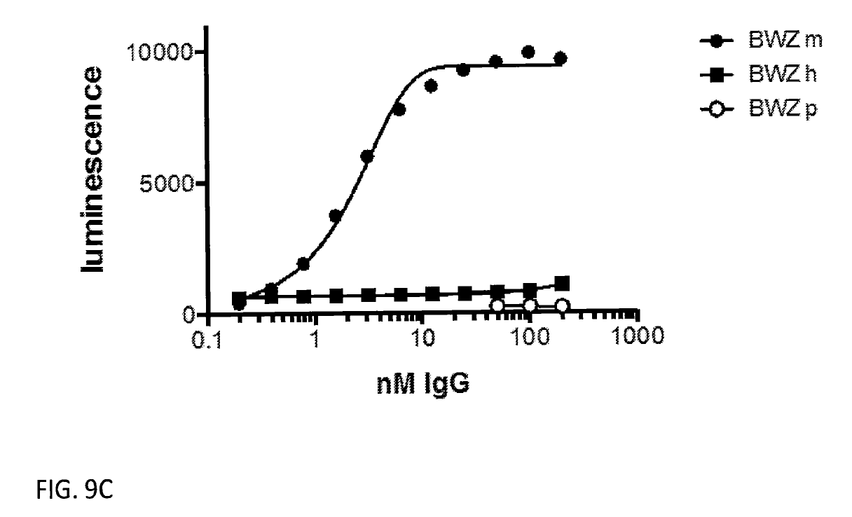

As shown in FIG. 9A, soluble full-length anti-TREM2 antibodies 2F6, 3A7, 3B10, 7E5, 8F8, and 11H5 increased luciferase activity in cells expressing mouse TREM2, as compared to the isotype control (msIgG1), indicating that the antibodies are agonist antibodies that are able to induce TREM2-dependent gene transcription. In contrast, soluble, full-length anti-TREM2 antibodies 1H7, 2H8, and 7F8 appeared to act as antagonists to block tonic TREM2 signaling. The dotted line in FIG. 9A indicates the levels of TREM2 activity without stimulation.

As shown in FIG. 9B, anti-TREM2 antibodies 9F5, 12F9, 2C7, 2F5, 3C1, and 4D7 increased luciferase activity in cells expressing human TREM2, as compared to the isotype control (msIgG1), indicating that the antibodies are agonist antibodies that are able to induce TREM2-dependent gene transcription. In contrast, soluble full-length anti-TREM2 antibodies, such as 10A9 and 10C1, appeared to act as antagonists to block tonic TREM2 signaling. The dotted line in FIG. 9B indicates the levels of TREM2 activity without stimulation.

FIG. 9C shows a dose response curve of luciferase activity induced by increasing concentrations of soluble full-length anti-TREM2 antibody 7E5, indicating that the effect on gene expression is dose dependent and that the EC₅₀ is approximately 1.52 nM.

Taken together with the results in FIGS. 8C and 8D, the results in FIG. 9A-9C indicate that soluble agonist anti-TREM2 antibodies can induce gene expression to an extent that is similar to plate-bound phosphatidylserine (PS), which is believed to be a natural ligand of TREM2.

### Example 9: Analysis of the Ability of Soluble TREM2 Antibodies to Enhance the Activity of Natural Ligands of TREM2

The ability of soluble full-length anti-TREM2 antibodies to enhance the activity of natural ligands of mouse TREM2 or human TREM2 was evaluated using a luciferase reporter gene under the control of an NFAT (nuclear factor of activated T-cells) promoter to measure activation of gene expression. The cell line BW5147.G.1.4 (ATCC® TIB48TM), derived from mouse thymus lymphoma T lymphocytes, was infected with mouse TREM2 and DAP12, and with Cignal Lenti NFAT-Luciferase virus (Qiagen). Alternatively the BW5147.G.1.4 cell line was infected with a human TREM2/DAP12 fusion protein, and with Cignal Lenti NFAT-Luciferase virus (Qiagen). Cells were incubated for 6 hours, together with soluble anti-TREM2 and isotype control antibodies, on plates that were pre-coated with increasing concentrations of phosphatidylserine (PS) or sphingomyelin (SM). Luciferase activity was measured by adding OneGlo Reagent (Promega) to each well and incubating 3 min at room temperature on a plate shaker. Luciferase signal was measured using a BioTek plate reader.

Additionally, the ability of soluble full-length anti-TREM2 antibodies to enhance APOE3 binding to recombinant human TREM2 protein was evaluated by ELISA. 1 μg/ml of ApoE protein was coated on high binding ELISA plates overnight at 4° C. On day 2, the plates were washed three times with 0.05% Tween in 1×PBS. Plates were then blocked with 3% milk at room temperature for 1 hour. The plates were incubated with 20 nM of TREM2-Fc protein and either 15 μg/ml or 5 μg/ml of the TREM2 antibody per well. Then plates were washed three times at room temperature and incubated with secondary antibody goat anti-human Fc HRP for 1 hour. Plates were washed again three times at room temperature and 100 μL of TMB was added to each well. After the reaction came to completion, 50 μL of 2N sulfuric acid was added per well to stop the reaction. Plates were read with a plate reader at 630 and 450 nm.

As shown in FIGS. 10A and 10B, the soluble full-length anti-TREM2 antibody 7E5 increased the potency and the maximal effect of phosphatidylserine (PS) in cells expressing mouse TREM2, as compared to the isotype control (msIgG1). 7E5 also increased the maximal effect of sphingomyelin (SM) in cells expressing mouse TREM2, as compared to the isotype control (msIgG1) (FIGS. 10C and 10D). These results indicate that antibody 7E5 was able to enhance TREM2-dependent gene transcription induced by PS and SM, which are believed to be natural ligands of TREM2.

As shown in FIG. 10E, anti-TREM2 antibodies 3A7, 2F6, 11H5, and 8F8 increased the maximal effect of phosphatidylserine (PS) in cells expressing mouse TREM2, as compared to the isotype control (msIgG1), indicating that these antibodies were able to enhance TREM2-dependent gene transcription induced by natural ligands, such as PS.

FIG. 10F shows that in contrast to agonistic anti-TREM2 antibody 7E5, a commercial anti-TREM2 antibody (R&D Cat#F7E57291) inhibits the activity of sphingomyelin (SM).

As shown in FIG. 11A, soluble full-length anti-TREM2 antibody 9F5 increased the maximal effect of phosphatidylserine (PS) in cells expressing human TREM2. This result indicates that antibody 9F5 was able to enhance TREM2-dependent gene transcription induced by PS, which is believed to be a natural ligand of TREM2. As a control, mouse IgG1 isotype antibody had no effect (FIG. 11B).

Similar to antibody 9F5, anti-TREM2 antibodies 7B3, 9G1, 9G3, 11A8, 12F9, 3B10, and 8F8 also increased the maximal effect of phosphatidylserine (PS) in cells expressing human TREM2, as compared to the isotype control (msIgG1) (FIGS. 11C and 11D). These results indicate that these antibodies were able to enhance TREM2-dependent gene transcription induced by natural ligands, such as PS.

As shown in FIGS. 11E and 11F, soluble full-length anti-TREM2 antibody 9F5 increased the strength of binding of a recombinant human TREM2 to APOE3 in an ELISA binding assay. The results indicate that antibody 9F5 was able to stabilize the binding to a natural ligand of TREM2. By contrast other antibodies, such as antibody 9G3, antagonized the binding of TREM2 to APOE3. As a control, a mouse IgG1 isotype antibody had no effect.

Together with the results in FIG. 8A-8F and FIG. 9A-9C, these results demonstrate that agonistic anti-TREM2 antibodies synergize with natural ligands of TREM2, such as PS, SM, and APOE, to enhance TREM2-dependent gene transcription, as the increased level in TREM2-dependent gene transcription induced by the combination of agonistic anti-TREM2 antibodies and TREM2 ligand was greater than the cumulative level in TREM2-dependent gene transcription that would be expected when the levels induced by the anti-TREM2 antibody alone and TREM2 ligand alone are added together.

### Example 10: TREM2 Antibodies Induce Macrophage Killing

The antagonistic functionality of soluble, non-cross-linked anti-TREM2 antibodies was evaluated in innate immune cells (e.g., bone marrow-derived macrophages).

Bone marrow-derived macrophages obtained from C57B16 mice were plated on non-tissue-culture-treated 96-well plates in the presence of 20 ng/ml M-CSF and 10 μg/ml of soluble, non-cross-linked anti-TREM2 antibodies 1H7, 2F6, 2H8, 2A7, 7E5, 7F8, 8F8, R&D (R&D Cat#F7E57291), or mouse IgG1 (mIgG1) or rat IgG2b (R IgG2b) as isotype controls. Each condition was plated in triplicate. Analysis of cell viability was performed using a CellTiter-Glo® kit (Promega) 3 days later. Plates were read with a BioTek Synergy™ Microplate Reader using GENS™ 2.04 software.

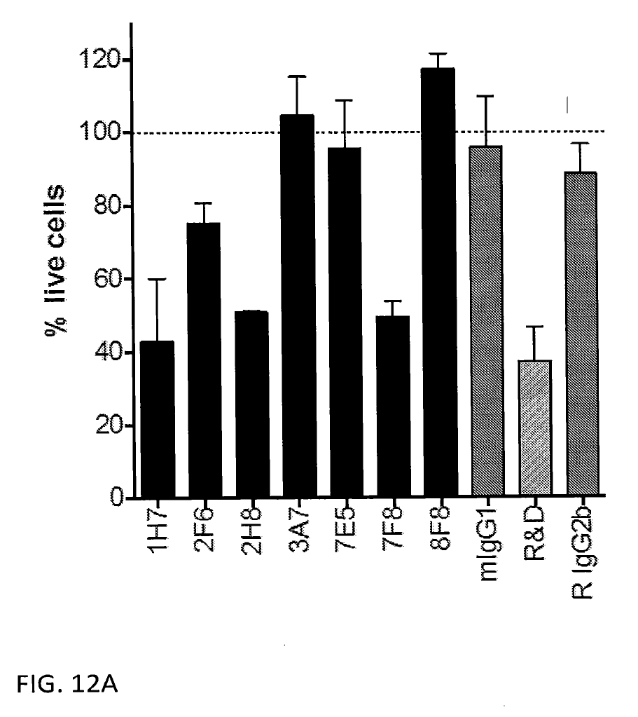

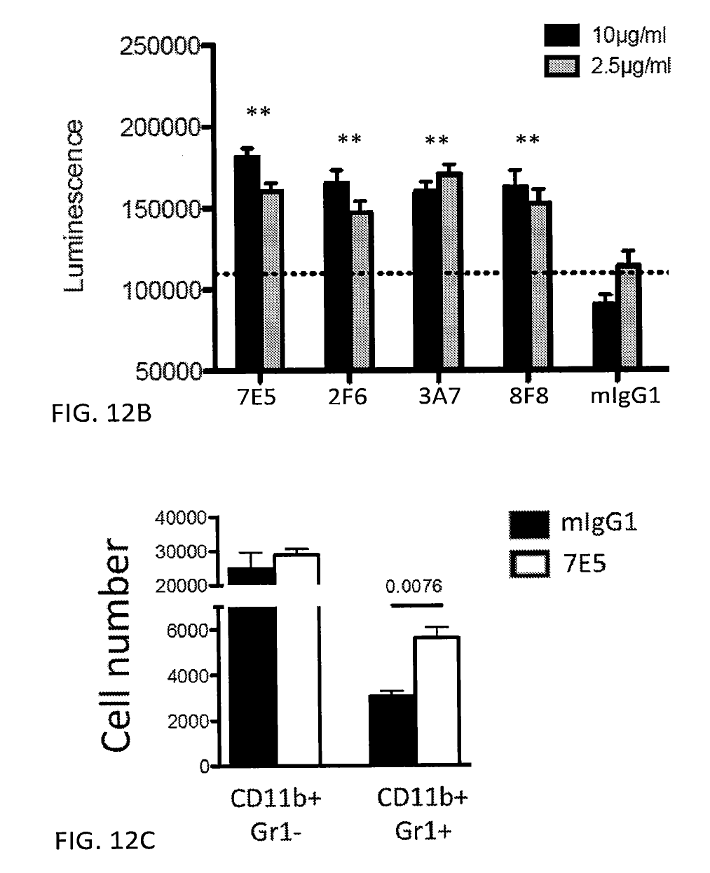

In FIG. 12A, the dotted line indicates the average cell viability obtained with untreated macrophages (i.e., no antibody added). The 0% reference indicates the average cell viability obtained when macrophages were cultured in the absence of M-CSF.

When macrophage cell viability was evaluated with soluble, non-cross-linked anti-TREM2 antibodies, the results indicated that antibodies 1H7, 2H8, and 7F8, and commercial antibody R&D decrease cell viability by ˜50% after 3 days of culture. By contrast, antibodies 3A7, 7E5, 2F6, and 8F8 do not have significant cytotoxic effects on primary macrophages.

### Example 11: TREM2 Increases Survival of Immune Cells

In Vitro Cell Survival

To evaluate the ability of anti-TREM2 antibodies to enhance cell survival in vitro, macrophages deficient in the gamma chain subunit of FcgRI, FcgRIII, and FceRI receptors (Fcgrl KO mice, REF: Takai T, Li M, Sylvestre D, Clynes R, Ravetch J. (1994). Cell, 76:519-529) were cultured in the presence of plate-bound anti-TREM2 antibodies and cell viability was determined when cells were cultured in suboptimal growth conditions.

Murine bone marrow precursor cells from FcgR1 KO mice (Taconic, Model 584) were obtained by flushing tibial and femoral marrow cells with cold PBS. After one wash with PBS, erythrocytes were lysed using ACK Lysing Buffer (Lonza), washed twice with PBS and suspended at 0.5×10⁶ cells/ml in complete RPMI media (10% FCS, Pen/Strep, Gln, neAA) with the indicated amount of M-CSF (Peprotech) to produce macrophages. To analyze cell viability of bone marrow-derived macrophages, cells were prepared as above and plated at 2.5×10⁴/200 μl in a 96-well plate with suboptimal amounts of M-CSF (10 ng/mil in non-tissue culture treated plates for two days. Cells were then quantified using the ToxGlo™ kit (Promega) and luminescence was determined as a measure of cell viability. All experiments were conducted in the presence or absence of anti-TREM2 antibodies or isotype control antibodies.

As shown in FIG. 12B, crosslinking the TREM2 receptor with plate-bound anti-TREM2 antibodies 7E5, 2F6, 3A7, and 8F8 increased the number of macrophages that are metabolically active in suboptimal culture conditions. In fact, incubation with plate-bound anti-TREM2 antibodies 7E5, 2F6, 3A7, and 8F8 increased cell viability by ˜50%, as compared the isotype control (mIgG1) and compared to non-treated macrophages (dotted line).

In Vivo Cell Survival

To evaluate the ability of anti-TREM2 antibodies to increase the number of immune cells in vivo, C57B16 mice were injected intraperitoneally (IP) with anti-TREM2 antibody 7E5 or a mouse IgG1 isotype control antibody, and the number of immune cells in the brain was then quantified by FACS.

Three to four mice per group received an IP injection of 40 mg/kg anti-TREM2 antibody 7E5 or isotype control antibody mIgG1 (clone MOPC-21, Bioxcell). 48 hours later, the entire brains were harvested, rinsed with PBS, incubated at 37° C. in PBS containing 1 mg/ml collagenase and processed through a cell strainer to obtain a single cell suspension. Cells were then incubated with anti-CD45-PerCp-Cy7, anti-CD11b-PerCP-Cy5.5, anti-Grl-FITC antibodies and a cell viability dye (Life Technologies, Cat# L34957) for 30 min on ice, then washed twice with cold FACS buffer. 4% PFA-fixed samples were then analyzed by FACS. Data were acquired on a BD FACSCanto™ II cytometer (Becton Dickinson) and analyzed with FlowJo software.

As shown in FIG. 12C, treatment with anti-TREM2 antibody 7E5 increased the number of immune cells that co-express the markers CD11b and Grl in the brain or blood vessels associated with the brain, as compared to treatment with the isotype control antibody. Treatment with anti-TREM2 antibody 7E5 alone induced the recruitment, on average, of 50% as many CD11b+Gr1+ cells (−6,000), as compared the isotype control or untreated cells (−4000 cells). Cells of the monocyte/macrophage lineage were defined as being positive for the surface markers CD11b and Grl.

### Example 12: Summary of TREM2 Agonistic Antibodies that Induce Gene Expression or Enhance Gene Expression Induced by Natural Ligands or by Binding to Natural Ligands

Tables 9A and 9B summarize results of the functional studies described in Examples 3-11 above. Anti-TREM2 antibodies demonstrated agonistic or antagonistic activity, either in solution or following antibody clustering (i.e., by plate binding), in modulating TREM2-dependent gene expression in cells expressing human TREM2 (Table 9A) or mouse TREM2 (Table 9B), as measured by a luciferase reporter gene or by modulating the strength of binding to TREM2. As indicated in Tables 9A and 9B, a subset of TREM2 antibodies displays agonistic activity when plate-bound. Another subset of TREM2 antibodies displays agonistic activity when in solution. A third subset of TREM2 antibodies displays antagonistic effects of soluble non-cross-linked antibodies. Certain anti-TREM2 antibodies increased binding of recombinant TREM2 protein to ligands (e.g., APOE3), while other anti-TREM2 antibodies decreased binding of recombinant TREM2 protein to ligands (e.g., APOE3).

In Table 9A, “Media” refers to a culture media only control, “mIgG1” refers to mouse IgG1 isotype control antibody, “mIgG2a” refers to mouse IgG2a isotype control antibody, “mIgG2b” refers to mouse IgG2b isotype control antibody, “rIgG1” refers to rat IgG1 isotype control antibody, “RIgG2a” refers to rat IgG2a isotype control antibody, “RIgG2b” refers to rat IgG2b isotype control antibody, “P+I” refers to a PMA/Ionomycin control, and “ND” refers to not determined.

TABLE 9A
TREM2 antibody functional studies with human TREM2

| | BWZhT2 LUC | | BWZhT2 LUC | BWZhT2 | |
|---|---|---|---|---|---|
| | Plate-bound | BWZhT2 LUC | Solution + PS | LUC Solution + | Percent human |
| Antibody | (FOB) | Solution (FOB) | (Δ Bmax) | PS (EC₅₀) | APOE3 binding |
| 1A7 | 3.36 | 0.96 | ND | ND | ND |
| 3A2 | 1.07 | 0.32 | ND | ND | ND |
| 3B10 | 5.80 | 0.89 | 1661 | 10.85 | ND |
| 6G12 | 2.11 | 0.30 | ND | ND | ND |
| 6H6 | 2.37 | 0.29 | ND | ND | ND |
| 7A9 | 2.01 | 0.86 | ND | ND | ND |
| 7B3 | 2.38 | 1.19 | 1645 | 4.41 | 64.3 |
| 8A1 | 5.46 | 0.35 | ND | ND | ND |
| 8E10 | 0.00 | 0.34 | ND | ND | ND |
| 8F11 | 1.62 | 0.26 | ND | ND | ND |
| 8F8 | 7.17 | 1.28 | 2235 | 10.27 | 79.7 |
| 9F5 | 2.48 | 1.75 | 2930 | 14.73 | 143.7 |
| 9G1 | 1.59 | 1.07 | 2657 | 5.82 | ND |
| 9G3 | 3.11 | 0.43 | 1258 | 7.82 | 16.4 |
| 10A9 | 2.18 | 0.31 | ND | ND | ND |
| 10C1 | 1.70 | 0.30 | ND | ND | ND |
| 11A8 | 6.01 | 0.79 | 957 | 8.45 | ND |
| 12E2 | 1.87 | 0.86 | ND | ND | ND |
| 12F9 | 9.29 | 2.04 | 1685 | 9.42 | ND |
| 12G6 | 2.70 | 0.90 | ND | ND | ND |
| 2C7 | 1.29 | 1.52 | ND | ND | ND |
| 2F5 | 0.92 | 1.61 | ND | ND | ND |
| 3C1 | 3.65 | 2.68 | ND | ND | ND |
| 4D7 | 5.72 | 2.22 | ND | ND | ND |
| 4D11 | 6.51 | 4.28 | −17661 | 8.7 | ND |
| 6C11 | 1.26 | 3.71 | ND | ND | ND |
| 6G12 | 1.20 | 1.14 | ND | ND | ND |
| 7A3 | 2.36 | 0.76 | ND | ND | ND |
| 7C5 | 5.95 | 4.00 | −34579 | 2.16 | ND |
| 7E9 | 5.17 | 2.19 | 25547 | 26.8 | ND |
| 7F6 | 4.24 | 3.45 | 133 | 3.85 | ND |
| 7G1 | 1.37 | 2.45 | ND | ND | ND |
| 7H1 | 2.70 | 1.96 | ND | ND | ND |
| 8C3 | 6.04 | 2.35 | −42068 | 2.24 | ND |
| 8F10 | 1.81 | 1.28 | ND | ND | ND |
| 12A1 | 1.61 | 0.80 | ND | ND | ND |
| 1E9 | 5.4 | 1.2 | −435 | 4.63 | ND |
| 2C5 | 5.6 | 1.2 | −4695 | 5.85 | ND |
| 3C5 | 5.6 | 1.3 | −888 | 6.44 | ND |
| 4C12 | 5.5 | 1.3 | 49 | 5.66 | ND |
| 4F2 | 4.2 | 1.3 | −4720 | 4.78 | ND |
| 5A2 | 3.2 | 3.1 | −47304 | 1.6 | ND |
| 6B3 | 1.8 | 0.7 | ND | ND | ND |
| 7D1 | 2.5 | 2.9 | −36088 | 1.8 | ND |
| 7D9 | 3.6 | 1.2 | −2853 | 5.55 | ND |
| 11D8 | 5.2 | 1.2 | ND | ND | ND |
| 8A12 | 1.3 | 1.1 | ND | ND | ND |
| 10E7 | 0.8 | 1.4 | ND | ND | ND |
| 10B11 | 5.1 | 1.0 | ND | ND | ND |
| 10D2 | 0.8 | 0.4 | ND | ND | ND |
| 7D5 | 1.1 | 1.0 | ND | ND | ND |
| 2A7 | 1.6 | 3.4 | ND | ND | ND |
| 3G12 | 1.1 | 1.4 | ND | ND | ND |
| 6H9 | 1.1 | 3.2 | ND | ND | ND |
| 8G9 | 1.7 | 2.9 | ND | ND | ND |
| 9B4 | 1.2 | 2.8 | ND | ND | ND |
| 10A1 | 1.3 | 1.4 | ND | ND | ND |
| 11A8 | 1.0 | 1.4 | ND | ND | ND |
| 12F3 | 0.9 | 1.3 | ND | ND | ND |
| 2F8 | ND | ND | ND | ND | ND |
| 10E3 | ND | ND | ND | ND | ND |
| 1B4 | 3.5 | 1.5 | ND | ND | ND |
| 6H2 | 1.1 | 3.6 | −40202 | 3.0 | ND |
| 7B11 | 5.5 | 1.2 | ND | ND | ND |
| 18D8 | 1.4 | 1.3 | ND | ND | ND |
| 18E4 | 1.7 | 1.2 | ND | ND | ND |
| 29F6 | 1.7 | 3.8 | ND | ND | ND |
| 40D5 | 1.9 | 1.4 | ND | ND | ND |
| 43B9 | 1.2 | 1.3 | ND | ND | ND |
| 44A8 | 1.5 | 6.6 | ND | ND | ND |
| 44B4 | 0.8 | 1.4 | ND | ND | ND |
| 45D6 | 2.3 | 1.0 | ND | ND | ND |
| 29F7 | 1.5 | 1.2 | ND | ND | ND |
| 32G1 | 1.0 | 1.1 | ND | ND | ND |
| Media | ND | ND | 0.00 | 5.04 | ND |
| mIgG1 | 0.9 | 1.3 | −383.67 | 6.59 | ND |
| mIgG2a | 1.0 | 1.4 | −1650.00 | 3.57 | ND |
| mIgG2b | 0.9 | 1.4 | −98.00 | 4.18 | ND |
| P + I | 26.5 | ND | ND | ND | ND |
| R IgG1 | 1.0 | 1.2 | ND | ND | ND |
| R IgG2a | 0.8 | 1.2 | ND | ND | ND |
| R IgG2b | 0.8 | 1.3 | ND | ND | ND |

In Table 9B, “mIgGl” and “rIgG2b” refer to isotype control antibodies, “NT” refers to not treated control, “RDT2” refers to commercial anti-TREM2 antibody from R&D (R&D Cat#F7E57291), “PMA” refers to PMA-only positive control, “ND” refers to not determined, and “BMMAc Killing Solution” refers to decreased bone marrow-derived macrophage cell viability (due to increased macrophage killing)

TABLE 9B
TREM2 antibody functional studies with mouse TREM2

| | BWZmT | | BWZmT | | | | | |
|---|---|---|---|---|---|---|---|---|
| | 2 LUC | BWZmT | 2 LUC | BWZmT2 | | | | |
| | Plate- | 2 LUC | Solution + | LUC | Percent | | | BMMAc |
| Anti- | bound | Solution | PS (Δ | Solution + | APOE | | pSyk/ | Killing |
| body | (FOB) | (FOB) | Bmax) | PS (EC₅₀) | binding | pSyk | DAP12 | Solution |
| 1H7 | 2.89 | 0.49 | ND | ND | | + | ND | ++ |
| 2F6 | 25.53 | 10.62 | 7900 | 3.94 | 74.34 | ++ | + | − |
| 2H8 | 2.89 | 0.68 | ND | ND | | | | ++ |
| 3A7 | 7.80 | 7.18 | 10248 | 1.98 | 92.28 | ++ | + | - |
| 3B10 | 2.19 | 3.36 | 14 | 12.95 | 79.30 | ++ | + | NT |
| 7E5 | 8.47 | 9.53 | 2274 | 3.084 | 83.76 | + | + | − |
| 7F8 | 5.78 | 0.66 | ND | ND | | + | + | + |
| 8F8 | 33.71 | 1.76 | 1957 | 6.73 | 81.64 | − | ND | − |
| 11H5 | 3.63 | 5.84 | 4240 | 8.023 | 83.68 | + | ND | NT |
| mIgG1 | 1.11 | 3.79 | 819.5 | 13.33 | 90.81 | ND | ND | ND |
| NT | 1 | 1 | 0 | 12.155 | | ND | ND | ND |
| RDT2 | ND | ND | 3103 | 12.07 | 106.47 | ND | ND | ND |
| rIgG2b | ND | ND | −850 | 12.21 | 102.44 | ND | ND | ND |
| PMA | 30.81 | 24.00 | ND | ND | | ND | ND | ND |

### Example 13: Analysis of the Effect of TREM2 Antibodies in Increasing Recruitment of Immune Cells and Induction of Pro-Inflammatory Signals In Vivo

Recruitment of Immune Cells

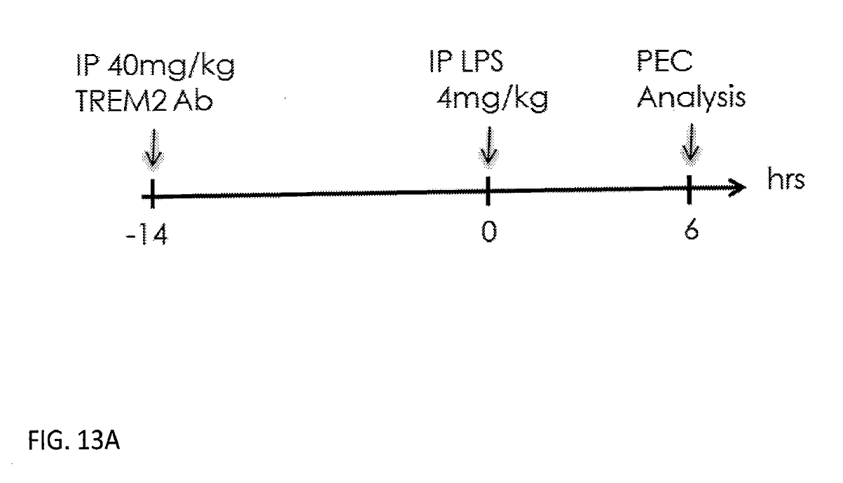

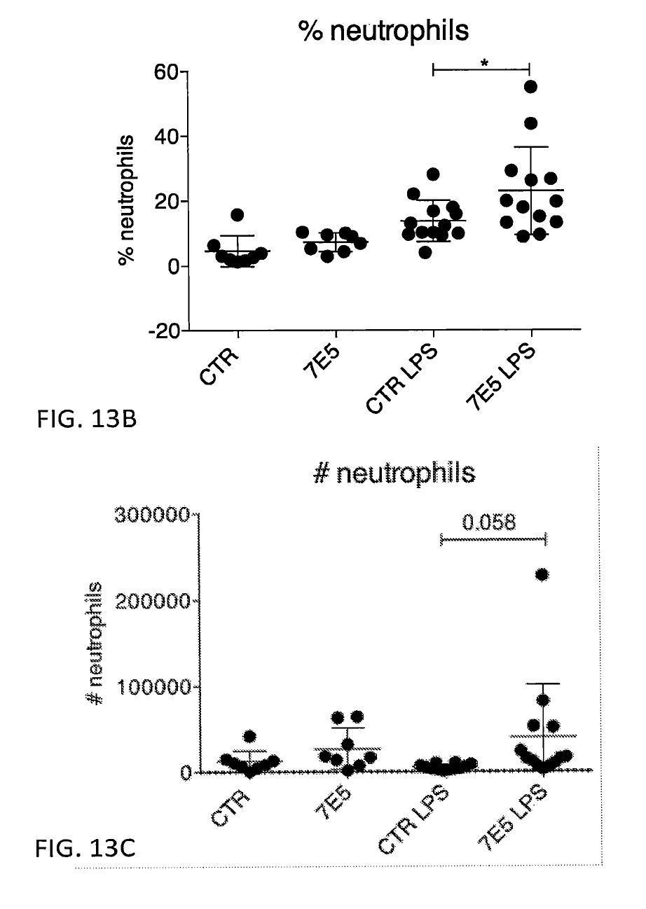

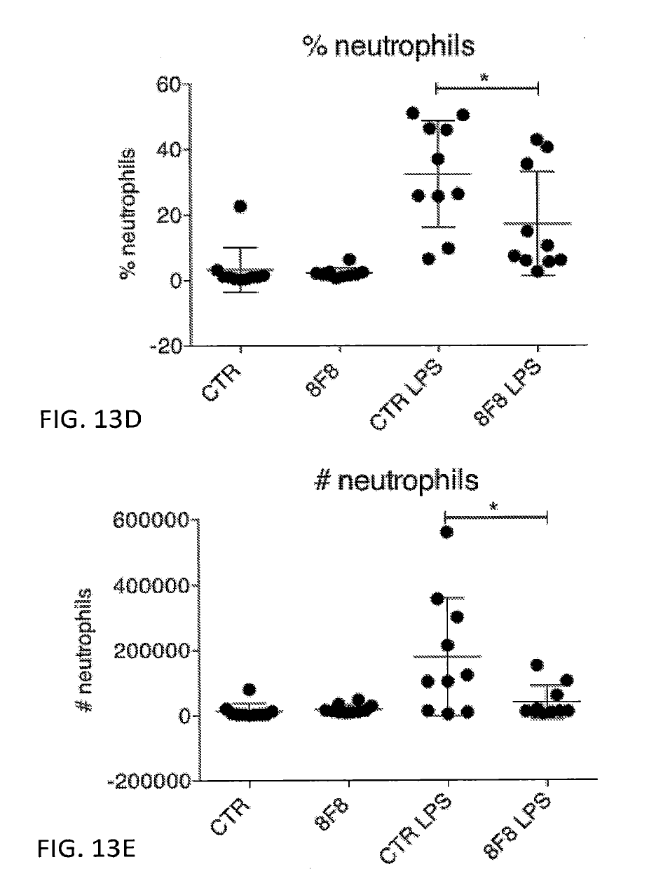

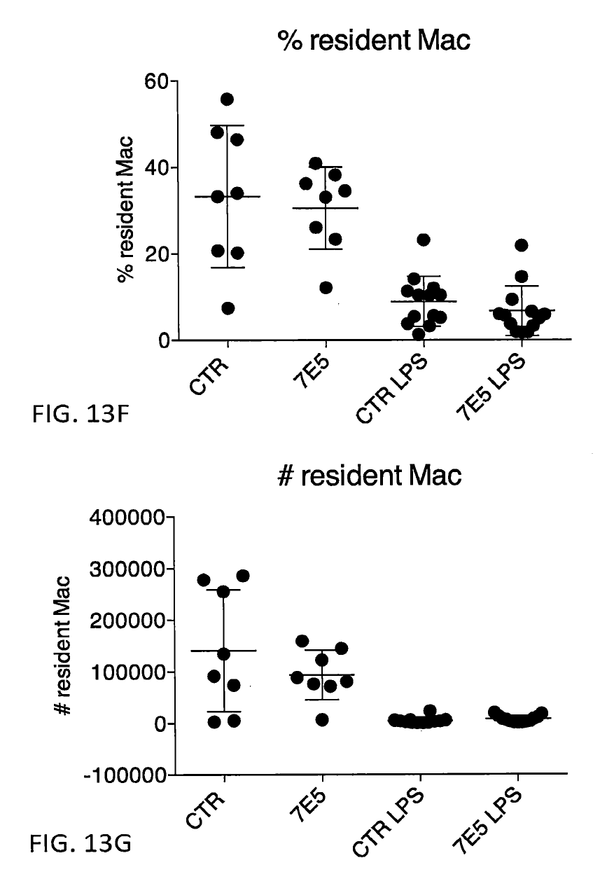

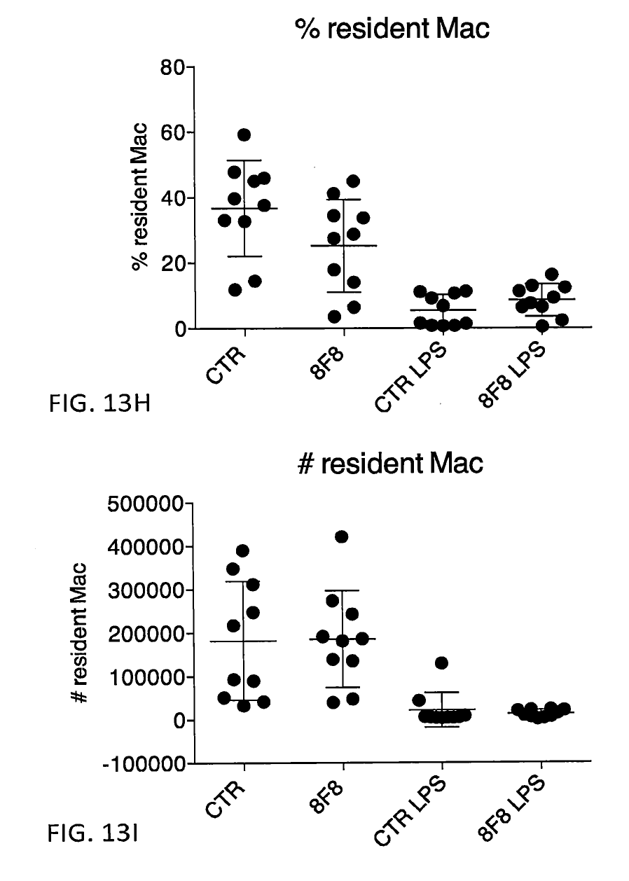

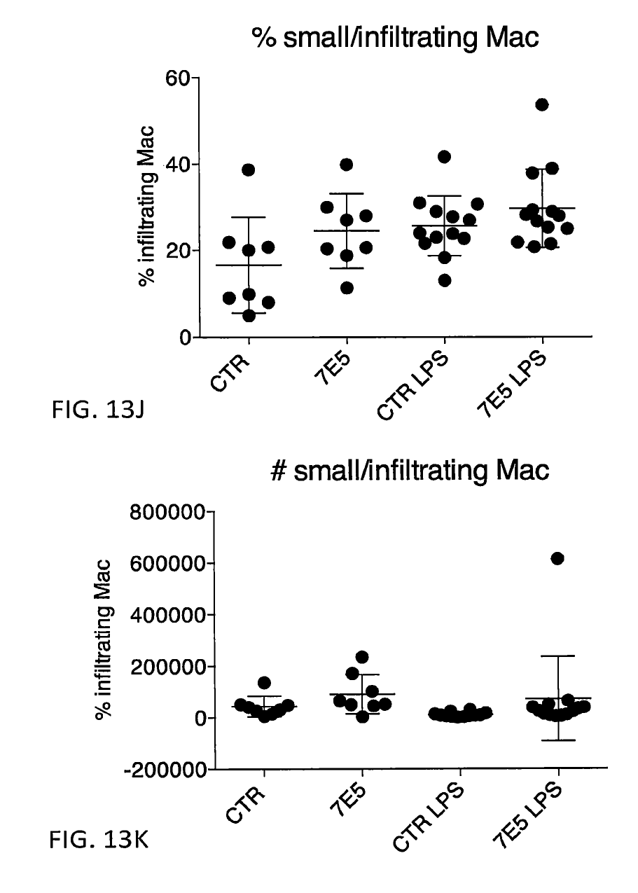

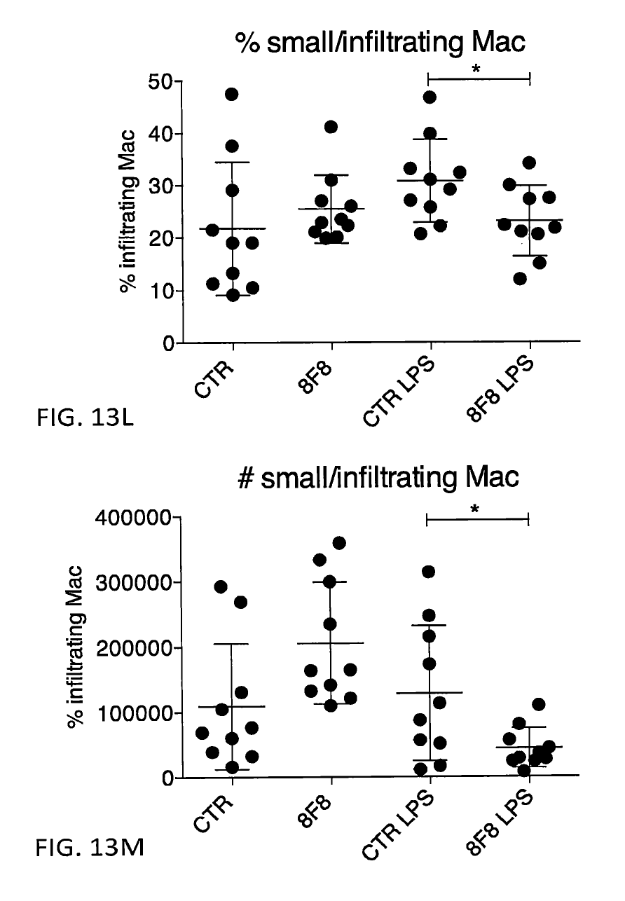

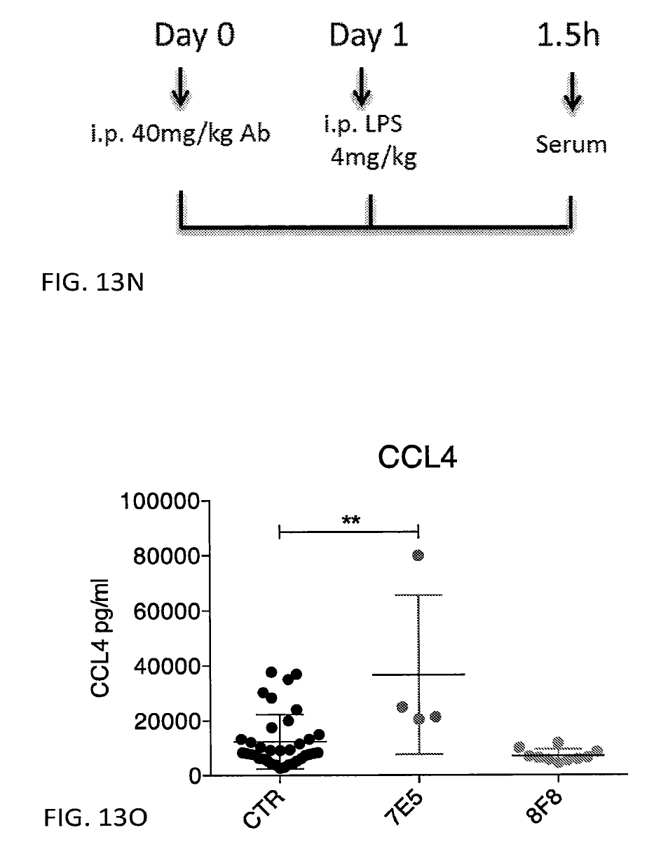

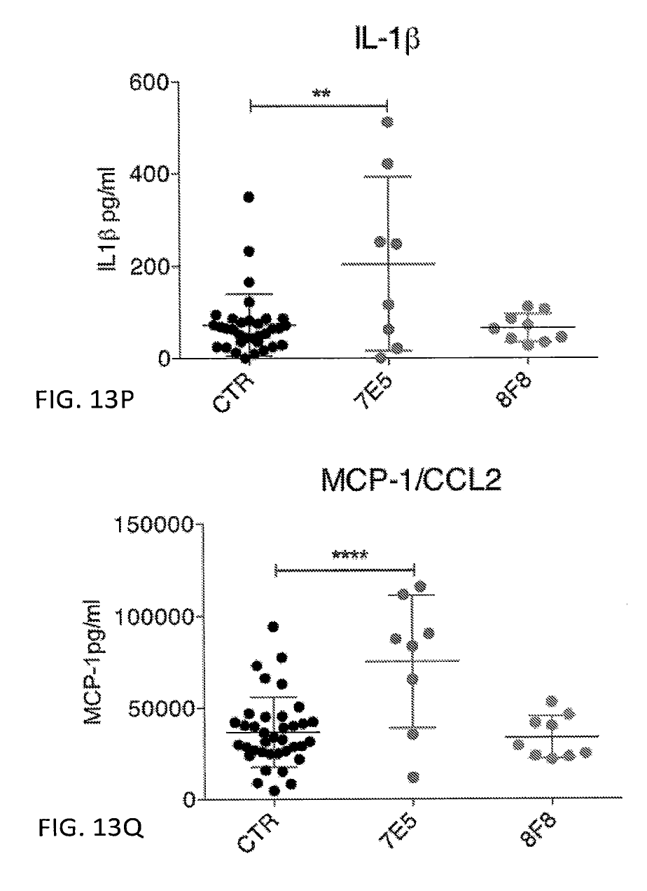

The ability of TREM2 antibodies to modulate the recruitment of inflammatory cells (neutrophil granulocytes, monocytes, and macrophages) in the peritoneal cavity (PEC) of C57B16 mice after intraperitoneal (IP) administration of either antibody alone or in combination with LPS was evaluated. Four mice per group were treated as described in FIG. 13A. Briefly, mice receive first an IP injection of 40 mg/kg anti-TREM2 antibody 7E5, antibody 8F8, or isotype control antibody mIgG1 (clone MOPC-21, Bioxcell). Fourteen hours later, mice received an IP injection of 4 mg/kg LPS, or PBS as a control. Six hours after LPS or PBS injection, cells were harvested from the PEC as described (see, e.g., Gawish R et al, 2014 FASEB J) and analyzed by FACS. For FACS analysis, PEC cells were incubated with anti-CD11b-Pacific Blue, anti-CD11c PeCy7, anti-MCH-II-APCCy7, anti-Grl-FITC, anti-Ly6G-PE and a viability die (Life Technologies, Cat# L34957) for 1 hour on ice, then washed twice with cold FACS buffer. 4% PFA-fixed samples were then acquired. Data were acquired on a BD FACS CANTO II cytometer (Becton Dickinson) and analyzed with FlowJo software.

As shown in FIGS. 13B and 13C, treatment with anti-TREM2 antibody 7E5 increased the number of neutrophils recruited in the PEC, as compared to treatment with the control antibody. The effect of anti-TREM2 antibody 7E5 on neutrophil recruitment was more pronounced in the presence of LPS, thus indicating that anti-TREM2 antibody 7E5 synergizes with LPS in the recruitment of neutrophils in the PEC. As shown in FIGS. 13D and 13E, treatment with anti-TREM2 antibody 8F8 did not increase the number of neutrophils recruited in the PEC, as compared to treatment with the control antibody. Neutrophils were defined as being positive for the surface markers Ly6G and Gr1.

As shown in FIG. 13F, 13G, 13 H, and 13I, treatment with anti-TREM2 antibody 7E5 or 8F8 did not increase the number of resident macrophages recruited in the PEC, as compared to treatment with the control antibody. Resident macrophages were defined as being positive for the surface marker CD11b and highly positive for the surface marker F4/80.

As shown in FIGS. 13J, 13K, 13L, and 13M treatment with anti-TREM2 antibody 7E5 and 8F8 increased the number of small infiltrating macrophages recruited in the PEC, as compared to treatment with the control antibody. The effect of anti-TREM2 antibody 7E5 and 8F8 on small infiltrating macrophage recruitment was not increased in the presence of LPS, indicating that anti-TREM2 antibodies 7E5 and 8F8 alone are sufficient to recruite small infiltrating macrophage in the PEC. Small infiltrating macrophages were defined as being positive for the surface marker CD11b and intermediately positive for the surface marker F4/80. Statistics in FIG. 13A-13M were calculated using Student's T-test, *Pval<0.05, **Pval<0.01, ***Pval<0.001.

Previous results have demonstrated that anti-TREM2 antibody 7E5 induces agonistic activity in vivo, and that anti-TREM2 antibody 8F8 behaves as a blocking antibody in vivo. The results in FIG. 13A to 13M, indicate that antibody 7E5 induces in vivo neutrophil accumulation in the peritoneum of mice that have been injected with LPS, while antibody 8F8 injection reduces the accumulation of neutrophils (and infiltrating macrophages) during LPS-induced sepsis. The results confirm that antibody 8F8 is a blocking antibody in vivo.

Induction of Pro-Inflammatory Signals

The ability of TREM2 antibodies to modulate the production of pro-inflammatory cytokines (CCL4, IL-1β, and MCP-1) in the peritoneal cavity (PEC) of C57B16 mice after intraperitoneal (IP) administration of either antibody alone or in combination with LPS was evaluated. Mice were treated as described in FIG. 13N. Briefly, mice received an IP injection of 40 mg/kg anti-TREM2 antibody 7E5, antibody 8F8, or isotype control antibody mIgG1 (clone MOPC-21, Bioxcell) on day 0. On day 1, mice received an IP injection of 4 mg/kg LPS, or PBS as a control. At 1.5 hours after LPS or PBS injection, the concentration of CCL4, IL-1β, and MCP-1 (CCL2) in serum samples from the mice was measured by cytometric bead assay (CBA).

As shown in FIGS. 13O to 13Q, anti-TREM2 antibody 7E5 induces enhanced production of CCL4, IL-1β, and MCP-1 in vivo. However, anti-TREM2 antibody 8F8 did not induce an in vivo increase in the production of CCL4, IL-1β, or MCP-1 (FIGS. 13O to 13Q).

### Example 14: TREM2 Antibodies Increase the Level of Soluble TREM2 in Mice

It is believed that the extracellular portion of TREM2 can be shedded into a soluble form (sTREM2), and thus can be detected in the plasma and cerebrospinal fluid (CSF). It is also believed that in individuals with Alzheimer's disease or frontotemporal dementia the amount of sTREM2 in the CSF is reduced compared to healthy control individuals.

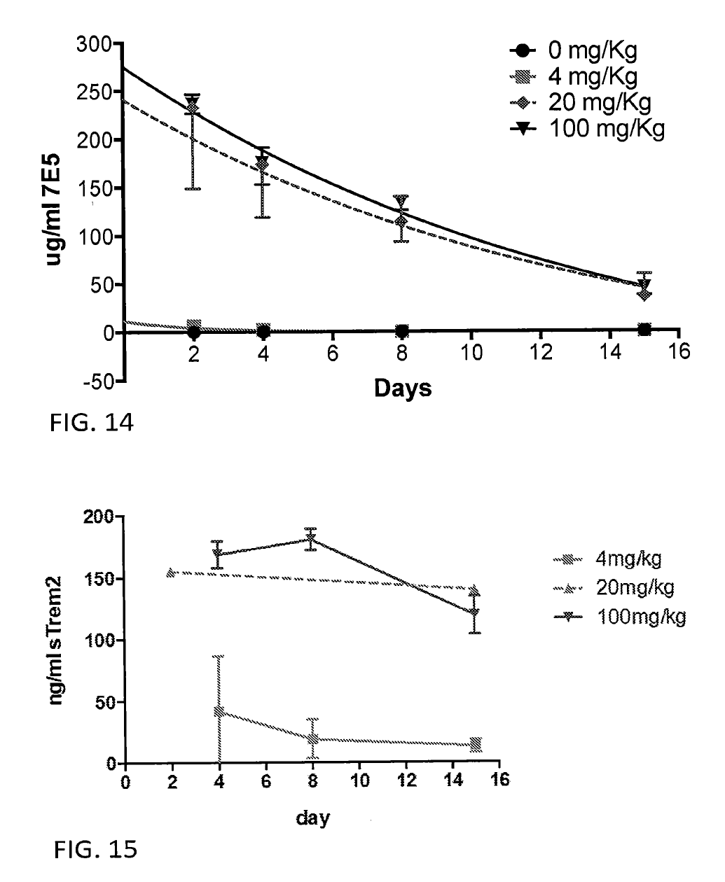

To determine the amount of anti-TREM2 antibodies present in the blood serum of mice at 2, 4, 8 and 15 days after injection of anti-TREM2 antibody 7E5, a standard ELISA method was utilized. Briefly, ELISA plates coated with 0.1 ug/well recombinant mouse TREM2 protein at 100 uL/well in carbonate coating buffer (pH 9.6) overnight at 4° C. Plates were then washed and blocked with 3% skim milk powder in PBS for 1 hour at room temperature and then washed. Mouse blood serum samples were titrated in PBS-Tween, added to the plate at 100 uL/well, and incubated for 1 hour at 37° C. with shaking. Anti-TREM2 antibodies were detected using a Goat anti-mouse IgG1-HRP secondary and developed with TMB substrate. A defined amount of anti-TREM2 antibody 7E5 was spiked in the blood serum of a naïve mouse and titrated to obtain a calibration curve. The results are depicted in FIG. 14, and indicate that the half-life of antibody 7E5 in the serum of mice is approximately 9.3 days.

To determine the effect of anti-TREM2 antibodies on blood serum levels of sTREM2 in mice, the amount of sTREM2 present in blood samples from mice was measured at 2, 4, 8 and 15 days after the injection of soluble anti-TREM2 antibody 7E5. Serum levels of sTREM2 were measured using a standard ELISA method. Briefly, Immulon ELISA 96-well plates were coated overnight at 4° C. with 100 μI of capture anti-TREM2 antibody (ADI-9) at 2 μg/ml. The next morning plates were washed three times with 200 μl wash buffer (PBS+0.05% Tween-20). Then plates were blocked by addition of 300 μl binding buffer (PBS+1% BSA) for 1 hr at room temperature on orbital shaker. Subsequently serum samples (1:12 dilution) and standards (recombinant mouse TREM2, R&D Systems) were added in 100 μl binding buffer, and plates were incubated at room temperature for 1 hr. Then plates were washed three times with 200 μl wash buffer. The detection biotinylated rat anti-TREM2 (R&D Systems, biotinylated with micro-NHS-Peg4-Biotinylation kit from Life Technologies Pierce) was added at 1:10,000 in 100 μl binding buffer and incubated for 1h at room temperature on orbital shaker. Then plates were washed three times with 200 μl wash buffer. 100 μl Streptavidin-HRP (R&D Systems) at 1:200 in binding buffer was added to the plates and incubated for 20 min on orbital shaker. Then plates were washed three times with 200 μl wash buffer and 100 μl TMB substrate (Life Technologies Pierce) was added and incubated on plate shaker until color developed. The reaction was stopped by addition of 50 μl sulfuric acid, and color was quantified using a Biotek Synergy H1 plate reader.

As shown in FIG. 15, the anti-TREM2 antibody 7E5 increases the serum half-life of sTREM2 in mice in a dose dependent manner. This increase in serum levels can serve as a biomarker for the biological activity of TREM2 antibodies.

### Example 15: TREM2 Antibodies Decrease Cell Surface Levels of TREM2

It is believed that antibodies that target certain ITIM/ITAM receptors expressed on the surface of immune cells can reduce the surface levels of the receptor on monocytes, macrophages, dendritic cells, and/or microglia.

The ability of anti-TREM2 antibodies to reduce cell surface expression of TREM2 on mouse primary bone marrow derived macrophages (BMDM) was evaluated. BMDM were cultured in 96-well tissue culture plates that were pre-coated with increasing concentrations of phosphatidylserine (PS) or sphingomyelin (SM), which are believed to be natural ligands of TREM2. Either a Syk inhibitor (R408) or 10 ug/ml of soluble anti-TREM2 antibodies or isotype control antibodies were added. 24 hours later, BMDM were analyzed by FACS for TREM2 expression on the cell surface. TREM2 expression was detected using a commercial anti-TREM2 antibody (R&D Cat#F7E57291).

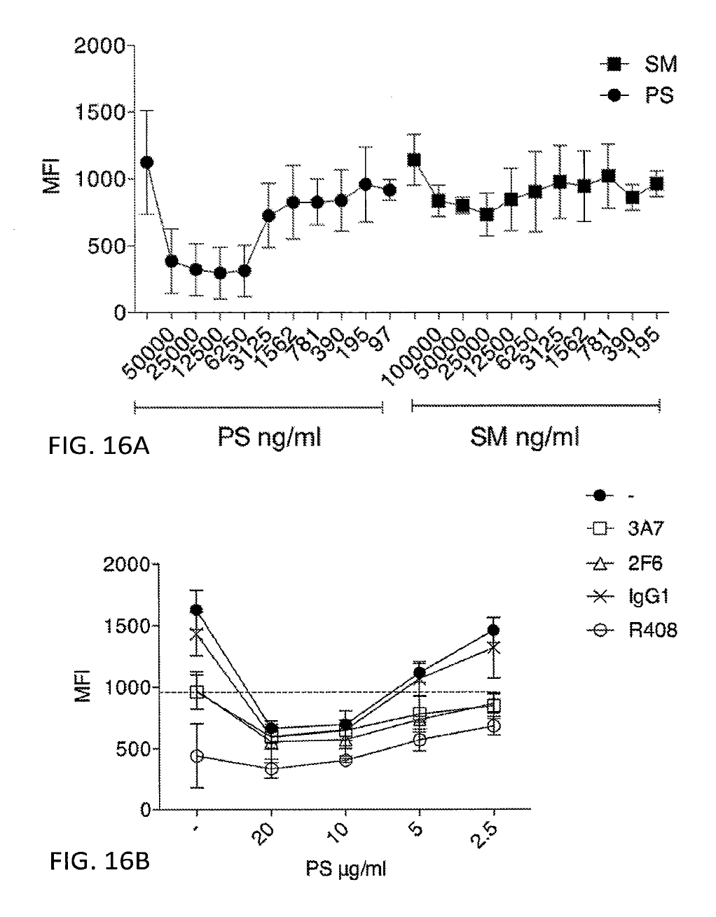

As shown in FIG. 16A, both TREM2 ligands PS and SM were able to decrease cell surface levels of TREM2 in a dose-dependent manner. Cell surface levels of TREM2 were reduced by ˜75% at the most effective PS dose (FIG. 16A). The effect of SM was less pronounced than PS. The maximal reduction of cell surface levels of TREM2 obtained with SM was ˜30% (FIG. 16A).

FIG. 16B demonstrates that treatment with soluble anti-TREM2 antibodies 3A7 or 2F6 alone decreased cell surface levels of TREM2 by ˜58%, which was similar to the reduction seen with the TREM2 ligands. R408 is a SYK inhibitor, which also decreases TREM2 cell surface levels. The results indicate that both activation of TREM2 signaling by lipid binding and inhibition induced by a signaling (SYK) inhibitor decrease cell surface levels of TREM2. Similarly, TREM2 antibodies 3A7 and 2F6 also decrease TREM2 cell surface levels.

### Example 16: Analysis of the Effect of Anti-TREM2 Antibodies in Mouse Models of Alzheimer's Disease

Mouse Models of Alzheimer's Disease

APP/PS1 mice contain human transgenes for both APP and PSEN1. The APP human transgene contains the Swedish mutation (K670N, M671L) and the PSEN1 human transgene contains an L166P mutation. Both transgene are under the control of the Thy1 promoter.

5XFAD mice overexpress mutant human APP (695) with the Swedish (K670N, M671L), Florida (1716V), and London (V717I) familial Alzheimer's disease (FAD) mutations. 5XFAD mice also overexpress human PS1 harboring two FAD mutations, M146L and L286V. Both transgenes are under the control of the mouse Thyl promoter to drive over expression in the brain and recapitulate major features of Alzheimer's disease.

Tg2576 mice overexpress a mutant form of APP (isoform 695) bearing the Swedish mutation (KM670/671NL).

For intracranial injections into 4-month old APP/PS1 or 5-month old 5xFAD mice, five mice per group received an injection of 2 ul of a 1 or 5 mg/ml solution of anti-TREM2 antibody 7E5 or isotype control antibody mIgG1 (clone MOPC-21, Bioxcell) as described (Wilcock D M, et al., (2003) J Neurosci 23:3745; Wilcock D M, et al., (2004) Neurobiol Dis 15:11; Sudduth et al., (2013) J. Neurosci, 33, 9684. On the day of surgery, mice were weighed, anesthetized with isoflurane, and placed in a stereotaxic apparatus (51733D digital dual manipulator mouse stereotaxic frame; Stoelting). A mid-sagittal incision was made to expose the cranium and four burr holes were drilled with a dental drill mounted in the stereotaxic frame over the frontal cortex and hippocampus to the following coordinates: frontal cortex, anteroposterior, +1.7 mm, lateral ±2.0 mm; hippocampus, anteroposterior −2.7 mm; lateral, ±2.5 mm, all taken from bregma. A 26 gauge needle attached to a 10 ml Hamilton syringe (Hamilton) containing the solution to be injected was lowered 3.0 mm ventral to bregma, and a 2 μl injection was made over a 2 min period. The incision was cleaned and closed with surgical staples. Three days post-injection, mice were perfused with saline and the right hemisphere of the brains was dissected into frontal cortex, hippocampus, rest of brain, and flash frozen. The left half was immersion fixed in freshly prepared 4% paraformaldehyde.

For systemic treatment of 3-month old wild-type (WT) or 5xFAD transgenic mice, animals were injected intraperitoneally weekly for 16 weeks with 50 mg/kg 7E5 antibody or mouse IgG1 isotype control antibody. At the end of the experiment, mice were perfused with normal saline and whole brains were extracted. The left hemibrain was drop fixed in 4% paraformaldehyde for 24h, followed by immunohistochemistry. The right hemibrains were dissected into frontal cortex, posterior cortex, hippocampus and cerebellum and flash frozen in liquid nitrogen.

Analysis of Cytokine and Chemokine Expression after Anti-TREM2 Antibody Treatment in the Brain In Vivo

The ability of anti-TREM2 antibodies to modulate the expression of inflammatory genes in different regions of the brain of APP/PS1 mice and 5XFAD mice was evaluated after intracranial (IC) administration of anti-TREM2 antibodies.

RNA was extracted from left hippocampus using the Trizol Plus RNA Purification System (Ambion, Invitrogen) according to the manufacturer's instructions. RNA was quantified using the BioSpec Nano spectrophotometer (Shimadzu) and cDNA was reverse transcribed using the cDNA High Capacity kit (Applied Biosystems) according to the manufacturer's instructions. Real-time PCR was performed using the 384-well microfluidic card custom TaqMan® assays containing TaqMan® gene expression probes for genes of interest IL-1b, IL-6, TNFa, IL-12, YM-1, IL-1Ra, MRC1, IL-10, CD86, FCGR1B, CCL2, CCL3, CCR2, CXCL10, Gata3, Rorc, OPN, FLT1, CSF-1, MHC-II, AXL and TGFb (Applied Biosystems, Invitrogen). All gene expression data was normalized to 18S rRNA expression. Fold change was determined using ACT-method. Data are presented as mean±SEM. Statistical analysis is performed using the JMP statistical analysis program (SAS). Statistical significance was assigned where the p value was lower than 0.05. One-way ANOVA and two-way ANOVA were used, where appropriate, to detect treatment differences and differences within treatment groups along the time course.

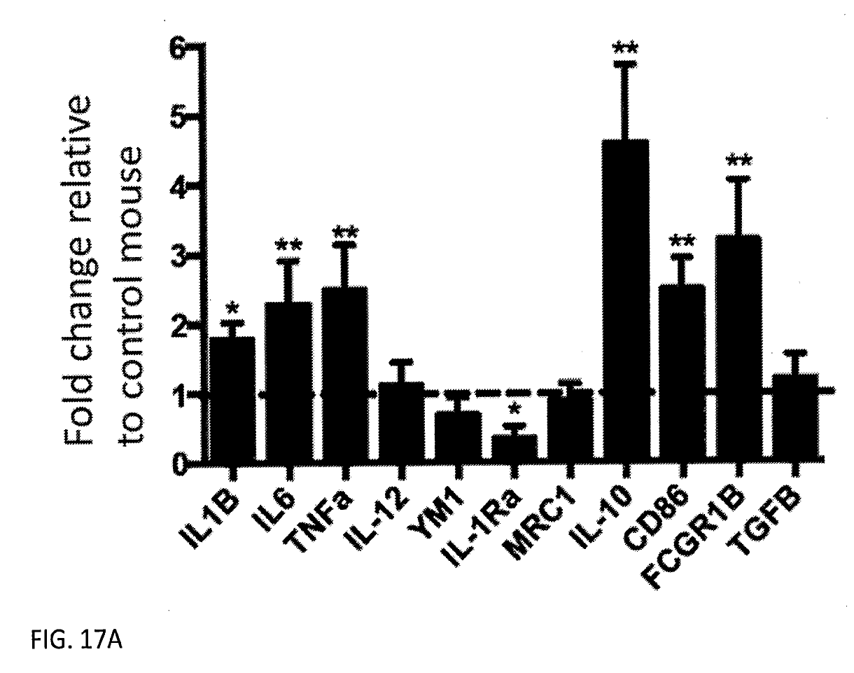

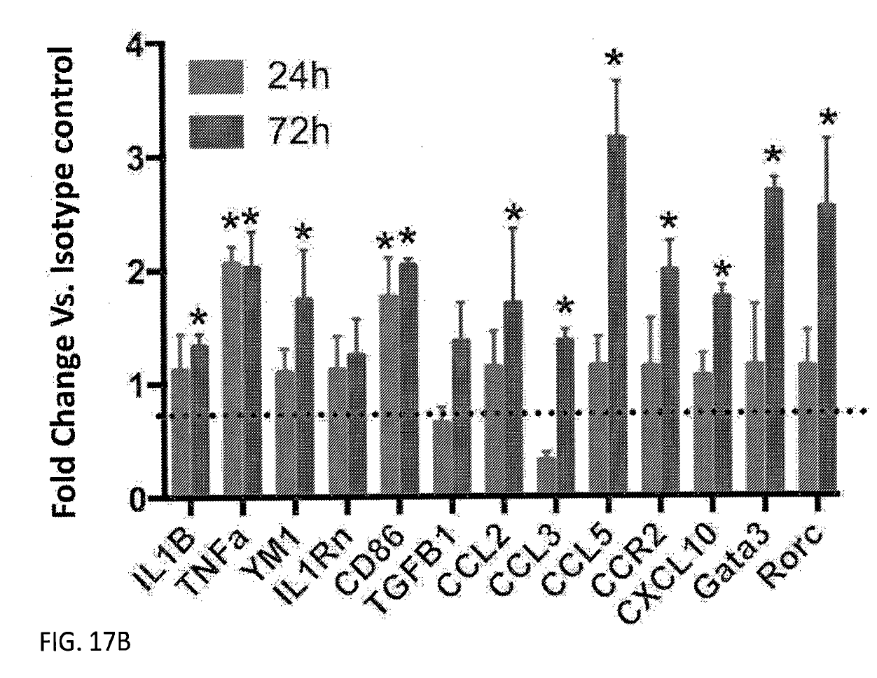

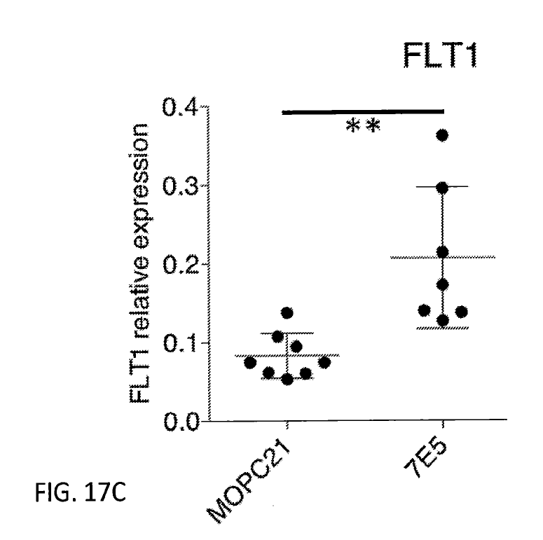

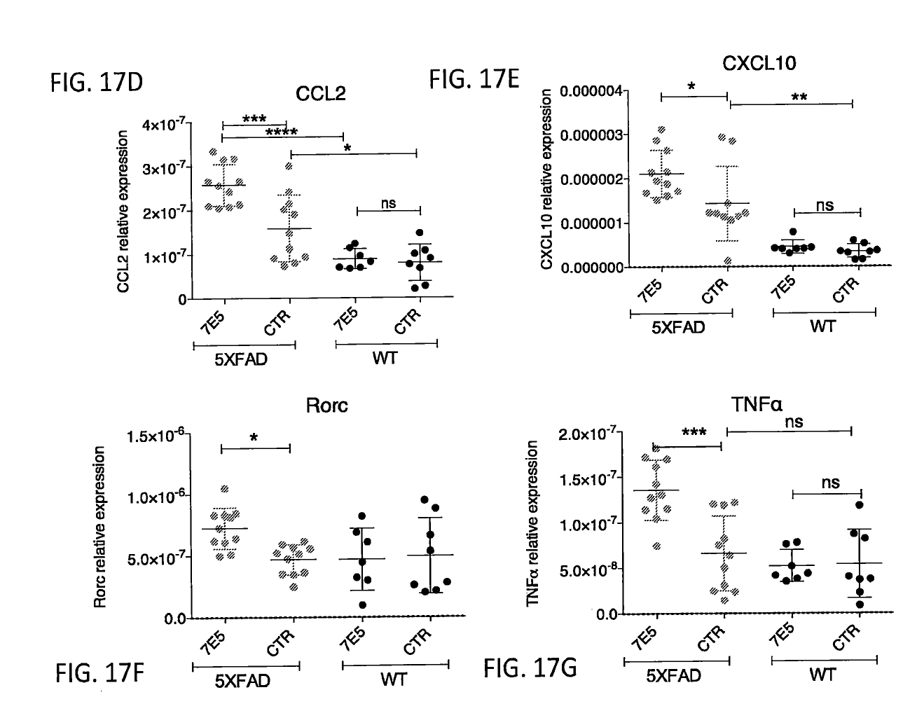

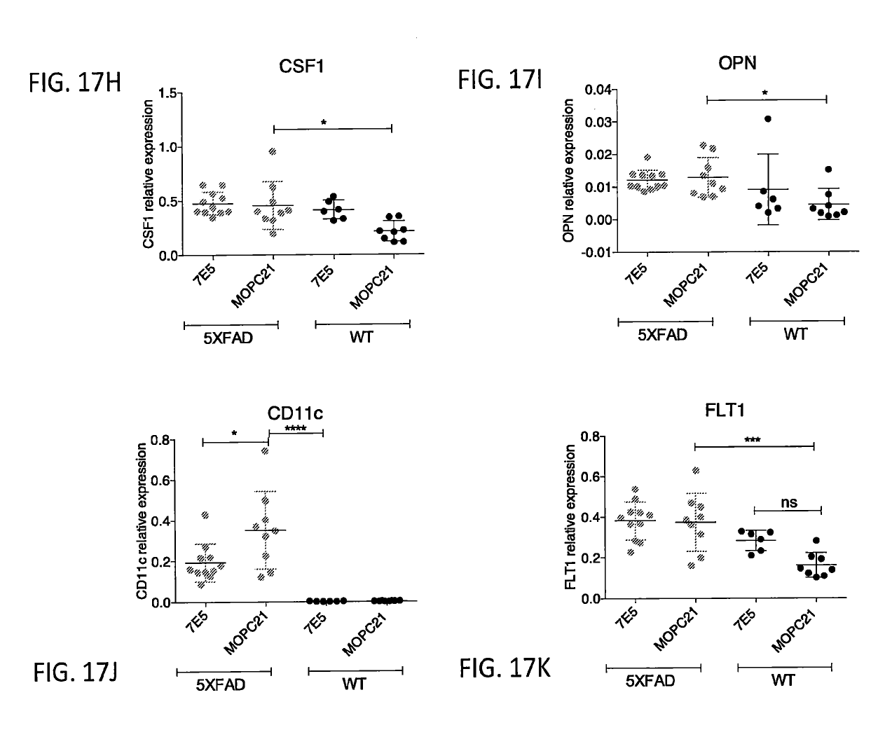

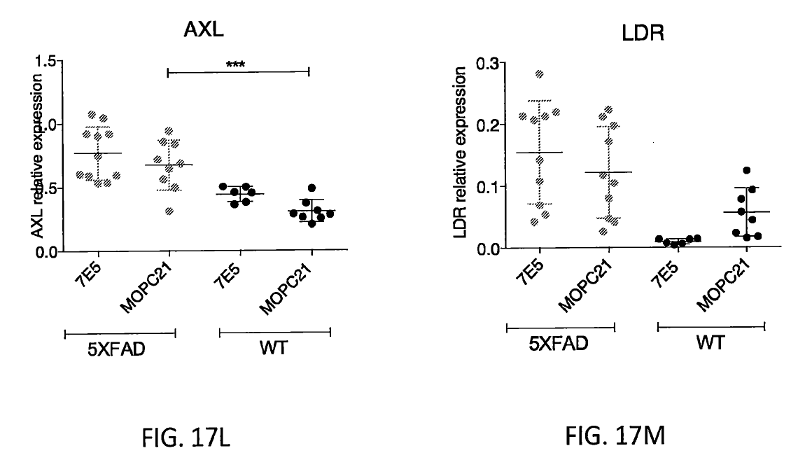

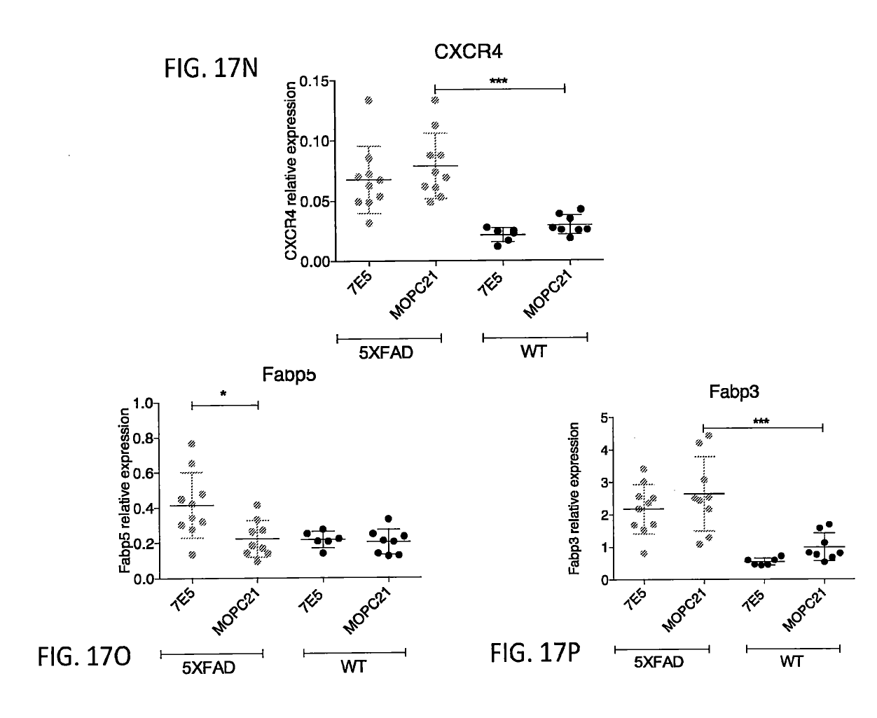

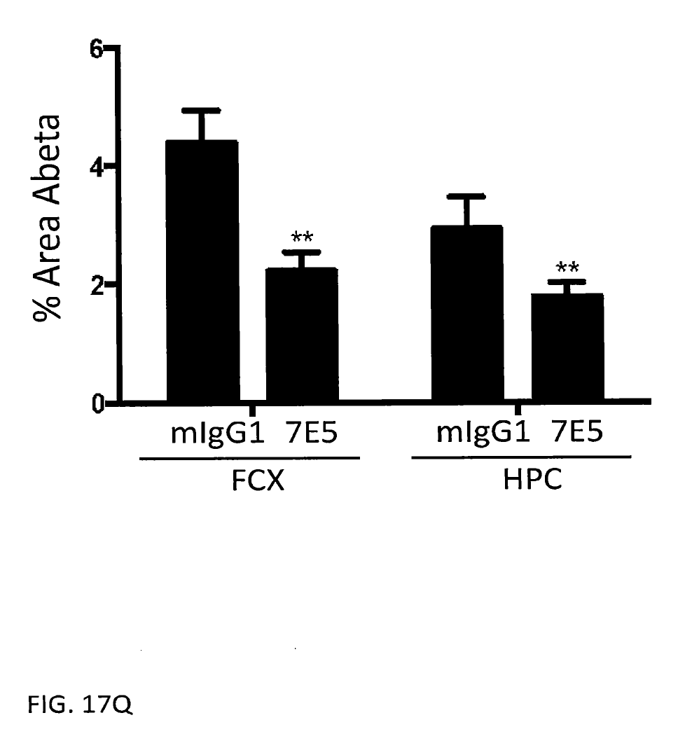

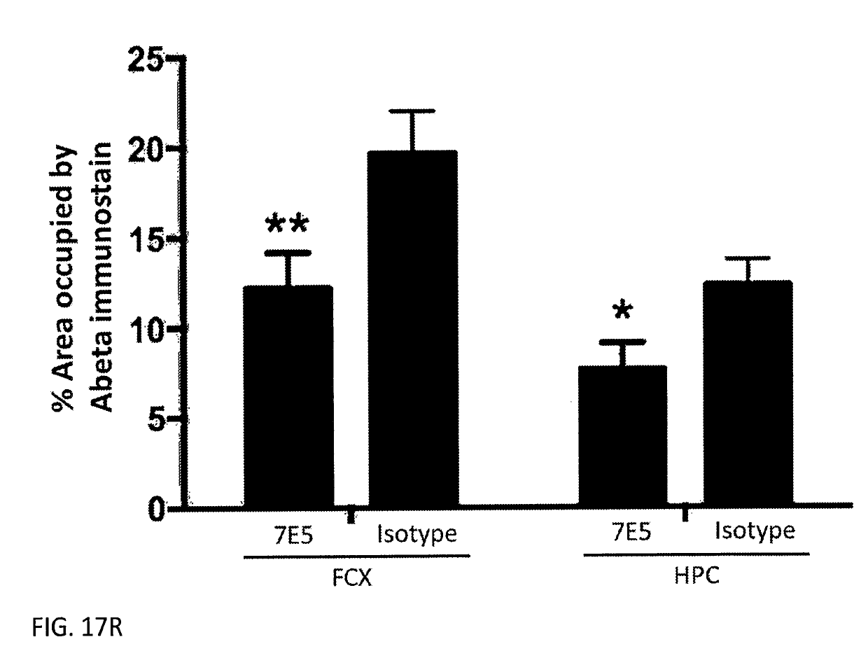

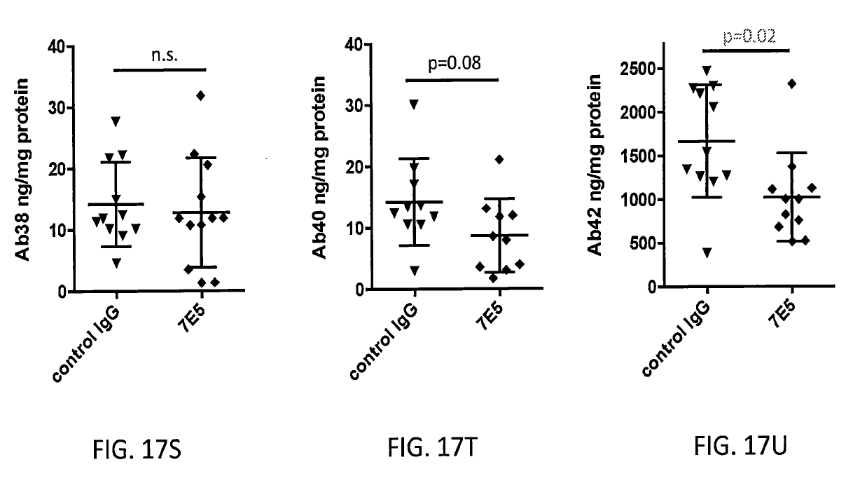

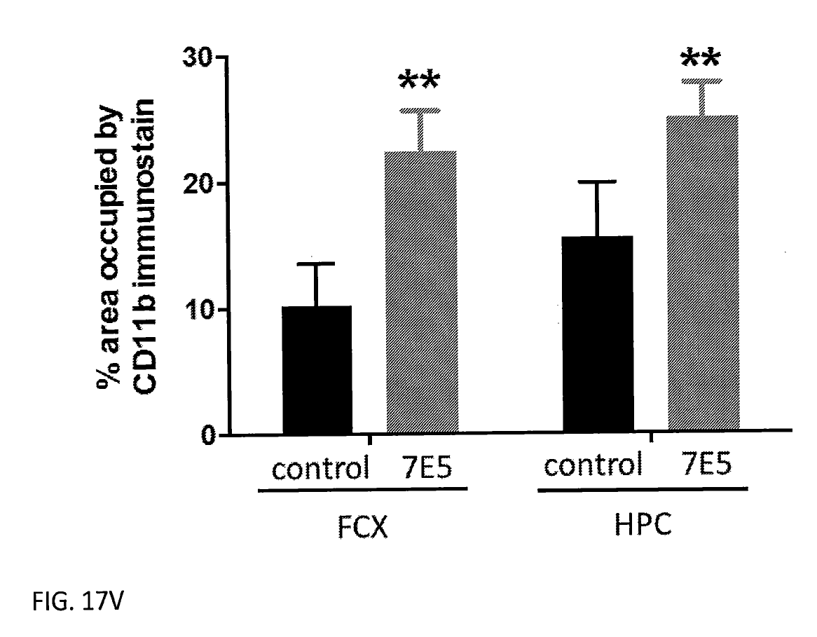

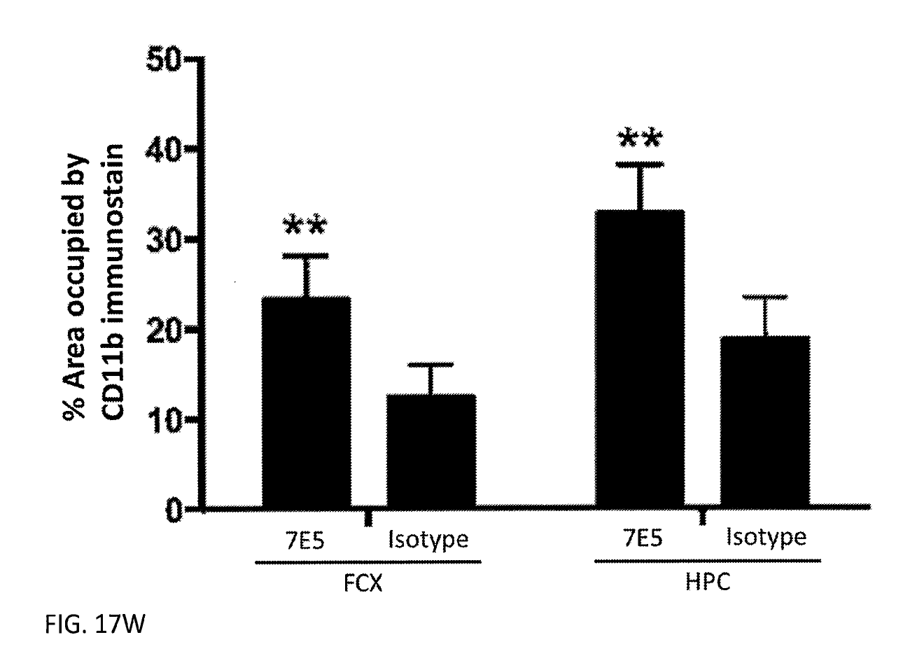

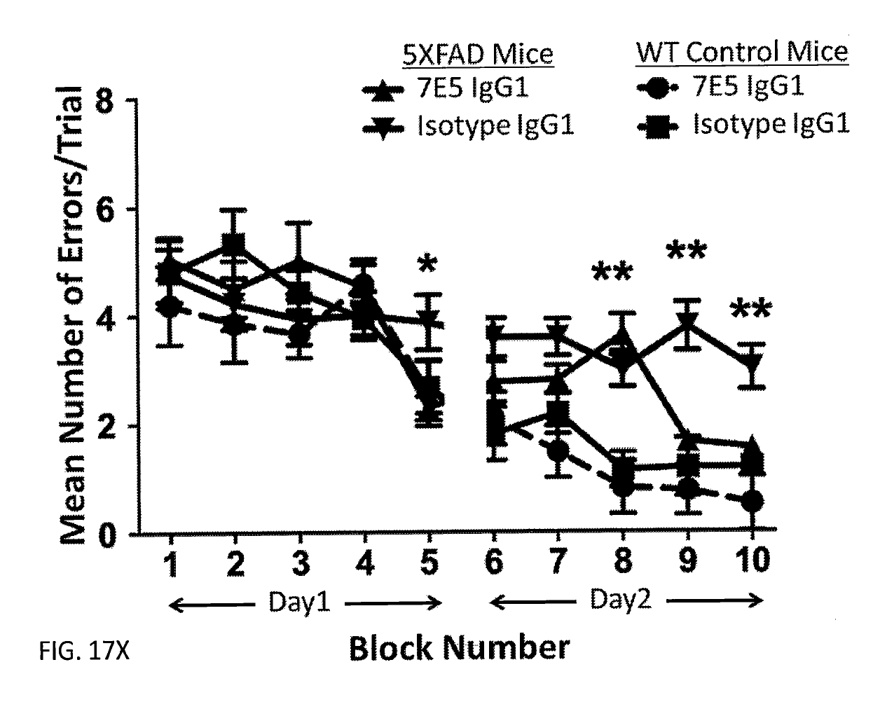

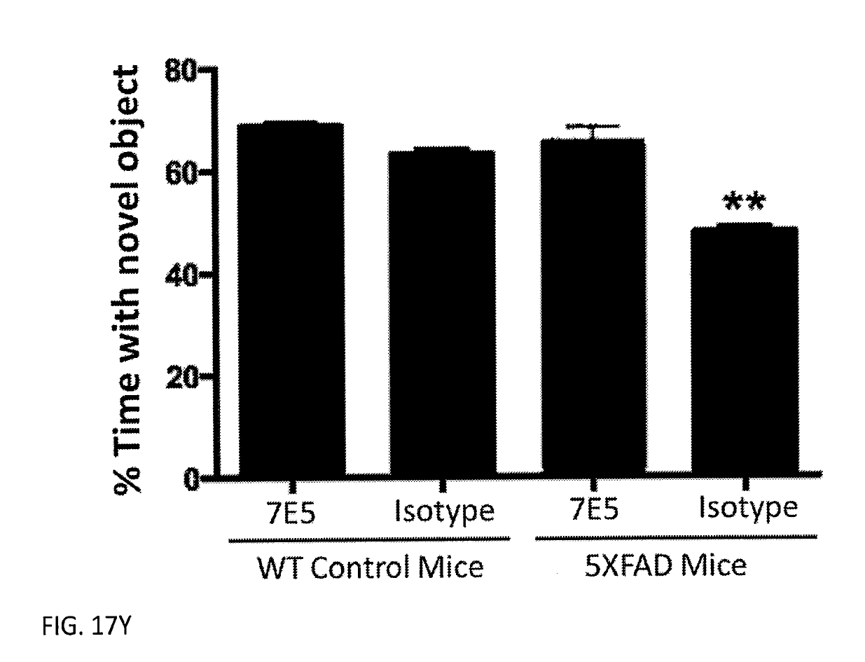

As shown in FIG. 17A, treatment of APP/PS1 mice with anti-TREM2 antibody 7E5 significantly increased the expression of IL-lb, IL-6, TNFa, and CD86 by approximately 2-fold. The expression of FCGR1B was increased approximately 3-fold, and the expression of IL-10 was increased approximately 4-fold. By contrast, expression of the IL-1Ra decreased by half. Expression of IL-12, YM-1, MRC1, and TGFB remained unchanged.

As shown in FIG. 17B, intracranial injection of 5XFAD mice with anti-TREM2 antibody 7E5 significantly increased the expression of IL-lb, TNFa, YM-1, CD86, CCL2, CCL3, CCR2, CXCL10, Gata3 and Rorc by approximately 2-fold 72 hours post injection. The expression of CCLS was increased approximately 3-fold. Expression of TGF-β1 remained unchanged. As shown in FIG. 17C, FLT1 levels were increased upon 7E5 injection. The expression of pro-inflammatory and anti-inflammatory genes in the hippocampus of 5XFAD mice 24 and 72 hours after injection with antibody 7E5 was measured using TaqMan assays containing TaqMan® gene expression probes for IL-1b, TNFa, YM-1, IL1Rn CD86, TGF-β1, CCL2, CCL3, CCL5, CCR2, CXCL10, Gata3, FLT1 and Rorc (Applied Biosystems, Invitrogen), and real-time PCR.

Three month long weekly injections of antibody 7E5 into 5xFAD mice decreased the level of CD11c, while increasing levels of Fabp3 (FIG. 17D-17P). In contrast, levels of CCL2, CXCL10, Rorc, Fabp5 and TNFa were increased. The results indicate that antibody 7E5 modulates inflammatory signaling and may thereby elicit a therapeutic benefit (FIG. 17D-17P).

Amyloid Beta Peptide Accumulation

The ability of anti-TREM2 antibodies to reduce the amount of amyloid beta (Abeta) peptide in different regions of the brain was evaluated after either intracranial (IC) administration of anti-TREM2 antibody into APP/PS1 mice or after intraperitoneal administration of anti-TREM2 antibody 7E5 into 5xFAD mice as described above. For the quantification of Abeta peptide, the paraformaldehyde fixed left hemibrain was passed through a series of 10, 20, and 30% sucrose solutions as cryoprotection and 25 μm frozen horizontal sections were collected using a sliding microtome and stored floating in PBS containing sodium azide at 4° C. Sections spaced 300 μm spanning the estimated injection site were initially mounted and stained by cresyl violet to identify the injection site. For all subsequent histology and immunohistochemistry six sections spanning the injection site, spaced 100 μm apart were selected and analyzed. Free-floating immunohistochemistry for Abeta (rabbit polyclonal antibody Aβ1-16; Invitrogen) was performed. The percent area occupied by positive stain was calculated using Nikon elements BR software.

To measure Abeta peptide levels in protein lysates, protein was extracted from the right frontal cortex using a two-step extraction method. First, the brain was homogenized in PBS containing a complete protease and phosphatase inhibitor (Pierce Biotechnology). These samples were centrifuged at 16,000×g at 4° C. for 1 h. The supernatant was removed and became the “soluble” extract. The resulting pellet was homogenized in 100 μl of 70% formic acid and centrifuged at 16,000×g at 4° C. for 1 h. The supernatant was removed and neutralized 1:20 with 1 M Tris-HCl and became the “insoluble” extract. Protein concentration for both the soluble and insoluble extracts was determined using the bicinchoninic acid protein assay according to manufacturer's instructions (Thermo Scientific). Meso-Scale Discovery multiplex ELISA system was used to measure Abeta 38 (A1338), Abeta 40 (A1340), and Abeta 42 (A(342) (MSD). ELISA kits were run according to the manufacturer's instructions.

As shown in FIG. 17Q, treatment with anti-TREM2 antibody 7E5 significantly decreased the area of the brain that stained positive for the Abeta peptide. In the frontal cortex Abeta peptide covered approximately 4% of the tissue in mice treated with control antibody, as compared to approximately 2% of the tissue in mice that were treated with anti-TREM2 antibody 7E5. In the hippocampus Abeta peptide covered approximately 3% of the tissue in mice treated with control antibody, as compared to approximately 2% of the tissue in mice treated with anti-TREM2 antibody 7E5.

As shown in FIG. 17R, injection of 5xFAD mice with the anti-TREM2 antibody 7E5 significantly decreased the area of the brain that stained positive for the Abeta peptide. In the frontal cortex Abeta peptide covered approximately 20% of the tissue in mice treated with control antibody, as compared to approximately 12.5% of the tissue in mice that were treated with anti-TREM2 antibody 7E5. In the hippocampus Abeta peptide covered around approximately 12% of the tissue in mice treated with control antibody, as compared to approximately 7.5% of the tissue in mice treated with anti-TREM2 antibody 7E5.

As shown in FIG. 17S-17U, injection of 5XFAD mice with the anti-TREM2 antibody 7E5 significantly decreased the levels of Abeta42, but not Abeta30, peptide in the insoluble protein lysates. There was a small, but not significant decrease of Abeta40 peptide. FIG. 17S shows levels of Abeta38 peptide. FIG. 17T shows levels of Abeta40 peptide. FIG. 17U shows levels of Abeta42 peptide.

Microglia Immunostaining

The ability of anti-TREM2 antibodies to modulate the number, morphology, and activation status of microglia in different regions of the brain in APP/PS1 mice intracranial injected with anti-TREM2 antibody or 5xFAD mice peripherally injected weekly for three months with the anti-TREM2 antibody 7E5 was evaluated after treatment with anti-TREM2 antibody. Mice were treated as described above, perfused, and brain samples were processed for histology. Six sections spanning the injection site, spaced 100 μm apart were selected and analyzed. Sections were incubated in primary anti-CD11b antibody, 1:3,000 (Rat monoclonal, AbD Serotec, Raleigh, N.C.) in 4% goat serum in DPBS left at room temperature and then overnight at 4° C. Sections were then incubated in biotinylated secondary antibody for 2 hours. Goat anti-rabbit IgG was used for GFAP and goat anti-rat for CD11b, both at 1:3,000 (Vector Laboratories, Burlingame, Calif.). Amplification of the secondary antibody signal was achieved through incubation in advidin-biotin complex (ABC) (Vector Laboratories, Burlingame, Calif.) for 1 hour. For color development, the vector diaminobenzidine (DAB) peroxidase kit (Vector Laboratories, Burlingame, Calif.) was used according to the manufacturer's instructions. Sections were mounted onto slides, left to air-dry overnight, dehydrated in an ethanol gradient followed by xylene incubations, and cover-slipped using DPX mountant (Electron Microscopy Sciences, Hatfield, Pa.). The percent area occupied by positive stain was calculated using Nikon elements BR software.

As shown in FIG. 17V, IC injection with anti-TREM2 antibody 7E5 in APPPS1 mice significantly decreased the area of the brain that stained positive for CD11b. In the frontal cortex, CD11b positive cells occupied approximately 10% of the tissue in mice that were treated with control antibody, as compared to approximately 22% in mice that were treated with anti-TREM2 antibody 7E5. In the hippocampus, CD11b⁺ cells occupied approximately 14% of the tissue in mice that were treated with control antibody, as compared to approximately 22% of the tissue in mice that were treated with anti-TREM2 antibody 7E5.

As shown in FIG. 17W, systemic treatment of 5XFAD mice with anti-TREM2 antibody 7E5 significantly increased the area of the brain that stained positive for CD11b. In the frontal cortex, CD11b positive cells occupied approximately 12% of the tissue when mice were treated with control antibody, as compared to approximately 23% of the tissue when mice that were treated with anti-TREM2 antibody 7E5. In the hippocampus, CD11b+ cells occupied approximately 18% of the tissue when mice were treated with control antibody, as compared to approximately 32% of the tissue when mice were treated with anti-TREM2 antibody 7E5.

Cognitive and Motor Function Determination

To evaluate the ability of anti-TREM2 antibodies to delay, prevent, or reverse the cognitive deficits of Alzheimer's disease (AD), 5XFAD mice were used. The 5XFAD mice were treated weekly with 50 mg/kg anti-TREM2 antibody 7E5 or with isotype control antibody mIgG1 (clone MOPC-21, Bioxcell) for 12 weeks. At the end of treatment, mice were tested for reduction in cognitive deficits using the radial arm water maze and the novel object recognition test.

Radial Arm Water Maze

The radial arm water maze is a spatial learning and memory task, as described in Wilcock et al., (2006) The Journal of Neuroscience, 26:5340. Briefly, after 12 weeks of antibody treatment, the 5XFAD mice were subjected to a 2 d radial arm water maze paradigm, followed by 1 d of an open-pool visible platform task. The apparatus was a six-arm maze as described previously in Gordon et al., (2001). Neurobiol Aging 22:377. The radial arm water maze task was run as described previously in Wilcock et al., (2004). J Neuroinflammation 1:24. On day 1, 15 trials were run in three blocks of five. Mice were further run in cohorts of four mice, permitting a short rest between each trial (as the other three mice were run) and a longer break between the blocks, when another cohort of four mice were run. This permitted rapid testing of aged mice without development of fatigue. Moreover, the spaced practice of the trials appears to enhance the rate of acquisition compared with daily massed trials over 10-14 d. The start arm was varied for each trial, with the goal arm remaining constant for both days. For the first 11 trials, the platform was alternately visible and then hidden and remained hidden for the last four trials. On day 2, the mice were run in exactly the same manner as day 1, except that the platform was hidden for all trials. The numbers of errors (incorrect arm entries) were measured in each trial over a 1 min time frame. To avoid confounds caused by inactive mice, one error is assigned for every 20 s a mouse failed to make an arm selection. Each mouse's errors for three consecutive trials were averaged, producing five blocks of trials for each day. Trial blocks were analyzed statistically by ANOVA using StatView (SAS Institute, Cary, N.C.). After the 2 d of radial arm water maze, the mice received 15 trials in an open-pool task with a visible platform to identify whether poor scores in the radial arm maze could be attributed to sensory or performance deficits rather than memory impairment. In the current study, all mice performed well on the visible platform version of the maze, and none were excluded because of sensory or performance deficits.

As shown in FIG. 17X, treatment with anti-TREM2 antibody 7E5 significantly improved special learning and memory deficits in 5XFAD mice. hi the last blocks (9-10) 5xFAD mice averaged 3 errors when treated with control antibody, as compared to less than 1 error when treated with the anti-TREM2 antibody 7E5 (FIG. 17X).

Novel Object Recognition Test

The novel object recognition test (NORT) is a sensitive and reproducible test for measuring cognitive abnormalities in mouse models of Alzheimer's disease. 5XFAD mice were placed for a 1-hour habituation period in an open glass aquarium-like transparent box, each at a time, in a sound-isolated room. On the following day they were reintroduced in the box for 5 min with two identical clean plaster objects, placed in two different corners of the box to measure baseline activity. Four hours later, one of the objects was replaced with a new one of the same size and texture, and the mice were reintroduced for an additional 5 min into the same cage to test for novel object recognition. The time spent by the mouse in object exploration was recorded manually by an operator blinded to the different treatments. The cumulative time spent at each of the objects was recorded. Exploration of an object was defined as directing the nose to the object at a distance of 2 cm and/or touching it with the nose. The percentage of the total time that the animal spent investigating the new object out of total exploration time was the measure of recognition memory. At baseline, the mouse spent approximately an equal time at each of the two objects, since both are novel to the mouse. At test, the cognitively healthy mice identify the new object as “new”, remembering the old one, and therefore spend more time exploring the new object (˜70-75% of the time).

Alzheimer's disease develops in 5xFAD mice over time leading to impaired memory, thus shorter (than normal) percent of the time exploring the new object. Significance for NORT was tested using two-way ANOVA for repeated measures, and post hoc Fisher's PLSD test. Data are expressed as mean±s.e.m.

As shown in FIG. 17Y, an improvement in cognitive function was observed in 5XFAD mice that were treated with anti-TREM2 antibody 7E5. The 5XFAD mice that were treated with the control antibody spent only approximately 50% of the time exploring the novel object, which is indicative of highly unpaired cognitive function. By contrast, mice treated with the anti-TREM2 antibody 7E5 spent 67% of the time exploring the novel object, which is close to the normal cognitive function measured in wild-type (WT) mice, indicating almost full recovery. Post hoc Fisher's PLSD test was used for statistical analysis **=Pval<0.01.

Tg2576 Mouse Alzheimer's Disease Model

To evaluate the ability of anti-TREM2 antibodies to delay, prevent, or reverse the development of Alzheimer's disease (AD), Tg2576 mice are used. Tg2576 mice overexpress a mutant form of APP (isoform 695) bearing the Swedish mutation (KM670/671NL). Mice are treated weekly with 50 mg/Kg anti-TREM2 antibody 7E5 or with isotype control antibody mIgG1 (clone MOPC-21, Bioxcell) starting from 98-99 weeks of age. Mice are tested for Abeta plaque load with immunohistochemistry and by ELISA of tissue extracts. Mice are further tested for the number of microglia in the brain, and for reduction in cognitive deficit using Morris Water maze, a spatial learning and memory task, Radial Arm Water Maze, a spatial learning and memory task, Y Maze (quantifies spontaneous alternation as a measure of spatial cognition), novelty preference in in an open field, operant learning to assess learning and memory, and fear conditioning (mousebiology.org website; Wang et al., (2015) Cell. pii: S0092-8674(15)00127-0).

### Example 17: TREM2 Expression in the Tumor Microenvironment

Groups of 3 C57B16 or BALB/c mice (females, 8 weeks old) were challenged subcutaneously with 1×10⁶ MC38 or CT26 colon carcinoma cells, or EMT-6 murine mammary carcinoma cells, suspended in 100 ul PBS. Animals are anesthetized with isoflurane prior to implant. When the tumors reached a size of 700-1000 mm³, tumors were explanted to analyze TREM2 expression in the tumor microenvironment by FACS. As a comparison, the spleen of the tumor baring mice or control spleen of naïve mice were also analyzed. For expression analysis by FACS, tumor and spleens were incubated in PBS containing lmg/ml collagenase and then processed through a cell strained to obtain a single cell suspension. Cells were then incubated with anti-CD45-PerCp-Cy7, anti-CD11b-PerCP-Cy5.5, anti-CD3-PC, anti-Grl-FITC, anti-NK1.1-PE, anti-TREM2-APC antibodies and a viability die (Life Technologies, Cat# L34957) for 30 min on ice, then washed twice with cold FACS buffer. 4% PFA-fixed samples were then acquired. Data were acquired on a BD FACS CANTO II cytometer (Becton Dickinson) and analyzed with FlowJo software.

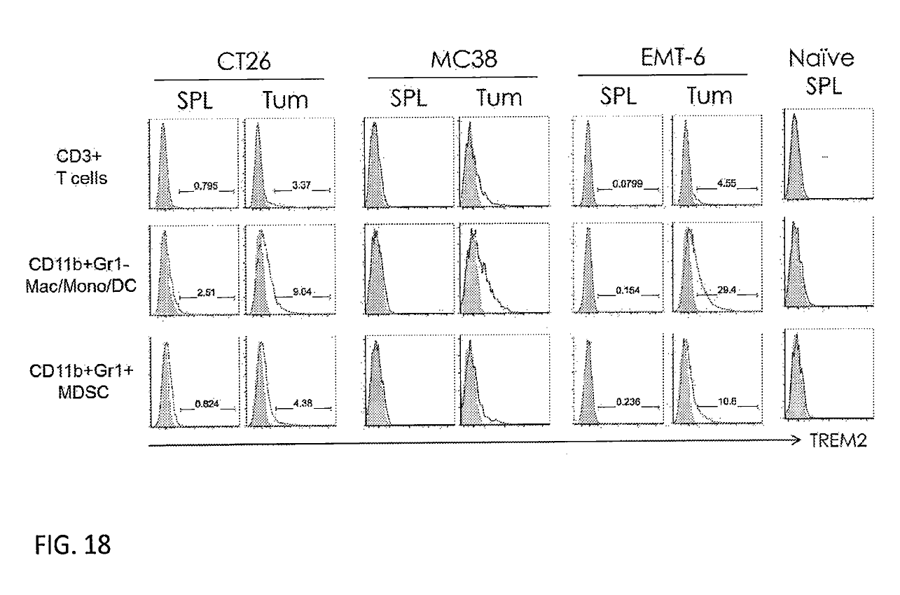

As shown in FIG. 18, TREM2 was found expressed on the cell surface in ˜5-20% of CD45+CD3-CD11b+Gr1-myeloid cells (which include macrophages, monocytes, and dendritic cells) and in ˜5-20% of CD45+CD3-CD11b+Gr1+ myeloid derived suppressor cells (MDSC) that infiltrate MC38, CT26 and EMT6 tumors. TREM2 was not found expressed in the spleen of tumor bearing mice or naïve mice. These results indicate that MC38, CT26, and EMT-6 tumors stimulate cell surface expression of TREM2 in a subset of myeloid cells.

### Example 18: Analysis of Tumor Growth in TREM2-Deficient Mice

Groups of TREM2 wild-type (WT, n=11) and TREM2 knock-out (KO, n=14) mice (sex and age-matched littermates, 10+/−2 weeks old) were challenged subcutaneously with 1×10⁶ MC38 colon carcinoma tumor cells suspended in 100 ul PBS. Mice were anesthetized with isoflurane prior to implant. Tumor growth was monitored with a caliper biweekly to measure tumor growth starting at day 5. The endpoint of the experiment is a tumor volume of 2000 mm³ or 60 days. Tumor size over time (expressed as volume, mm³) is the outcome measure.

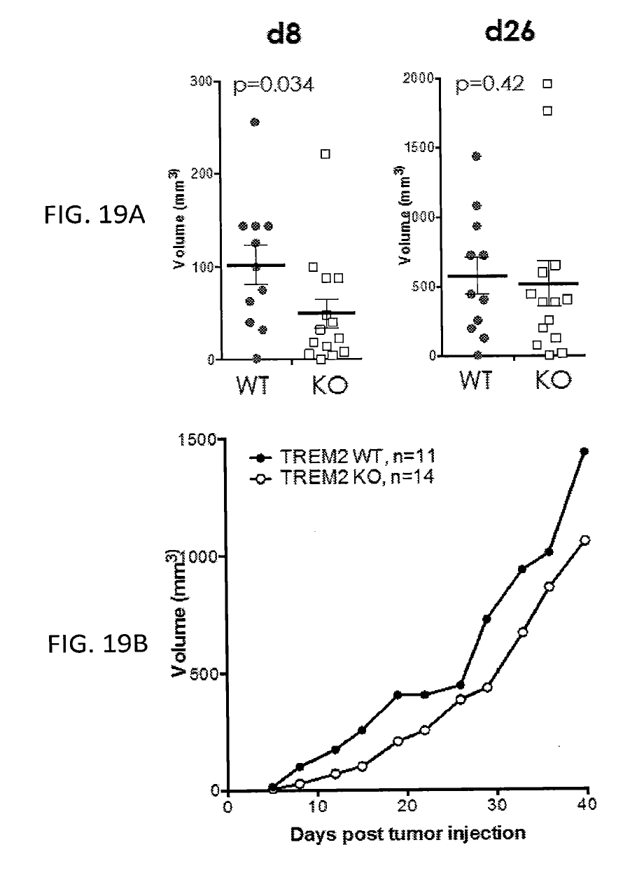

FIG. 19A shows that the average tumor size is significantly smaller in TREM2 knock-out (KO) mice, as compared to wild-type (WT) mice, at an early time point after tumor injection (day 8), while the difference in tumor size becomes no longer statistically significant at later time points (day 26). Median tumor growth was reduced in TREM2 knock-out (KO) mice, as compared wild-type (WT) mice (FIG. 19B). These results suggest that TREM2 promotes tumor growth, and is particularly prominent in the early phases of tumor progression.

### Example 19: Analysis of the Anti-Cancer Effect of TREM2 Antibodies in a Mouse Model of Breast Cancer

Groups of 10 BALB/c mice at 8 weeks (+/−2 weeks) of age are challenged subcutaneously with 5×10⁶ EMT-6 tumor cells suspended in 100 ul PBS. Animals are anesthetized with isoflurane prior to implant. Starting at day 2, groups of mice are injected IP at day 1, 4, 8, 15, and 22 with 40 mg/kg of anti-TREM2 antibodies. Tumor growth is monitored with a caliper biweekly to measure tumor growth starting at day 4. The endpoint of the experiment is a tumor volume of 2000 mm³ or 60 days. Tumor growth and % survival are the outcome measures. Reduced tumor take and growth rate, reduced number of tumor infiltrating immune suppressor macrophages, and increased effector T cell influx into the tumor indicate the anti-cancer effects of blocking anti-TREM2 antibodies.

### Example 20: Analysis of Additive Anti-Tumor Effect of Combination Therapy that Combines TREM2 Antibodies with Antibodies Against Inhibitory Checkpoint Proteins or Inhibitory Cytokines/Chemokines and their Receptors in a Mouse Model of Breast Cancer

Groups of 10 BALB/c mice at 8 weeks (+/−2 weeks) of age are challenged subcutaneously with 5×10⁶ EMT-6 tumor cells suspended in 100 ul PBS. Animals are anesthetized with isoflurane prior to implant. Starting at day 2, groups of mice are injected IP at day 1, 4, 8, 15, and 22 with 40 mg/kg of anti-TREM2 antibodies alone or in combination with antibodies against checkpoint proteins (e.g., anti-PDL1 mAb clone 10F.9G2 and/or anti-CTLA-4 mAb clone 9H10) at day 8 and 11. Treatment groups include anti-TREM2; anti-CTLA-4; anti-TREM2+anti-CTLA-4 and isotype control. Tumor growth is monitored with a caliper biweekly to measure tumor growth starting at day 4. The endpoint of the experiment is a tumor volume of 2000 mm³ or 60 days. Tumor growth and % survival are the outcome measures. A decrease in tumor growth and an increase in percent survival with combination therapy indicate that anti-TREM2 antibodies have additive or synergistic therapeutic effects with anti-checkpoint antibodies. Antagonistic antibodies against checkpoint molecules include antibodies against PDL1, PDL2, PD1, CTLA-4, B7-H3, B7-H4, HVEM, BTLA, KIR, GALS, TIM3, A2AR, LAG-3, and phosphatidylserine (PS). Antagonist antibodies against inhibitory cytokines include antibodies against CCL2, CSF-1, and IL-2.

### Example 21: Analysis of Additive Anti-Tumor Effect of Combination Therapy that Combines TREM2 Antibodies with Antibodies that Activate Stimulatory Checkpoint Proteins

Groups of 15 C57B16/NTac mice at 8 weeks (+/−2 weeks) of age are challenged subcutaneously with tumor cells as described in Example 19. Animals are anesthetized with isoflurane prior to implant. Starting at day 2, mice are injected intraperitoneally every 3 days for 4 doses with 200 ug anti-TREM2 antibodies alone or in combination with agonistic antibodies that activate stimulatory checkpoint proteins (e.g., OX40 or ICOS mAb) at day 3, 6, and 9. Tumor growth is monitored with a caliper biweekly to measure tumor growth starting at day 4. The endpoint of the experiment is a tumor volume of 2000 mm³ or 60 days. Tumor growth and percent survival are the outcome measures. A decrease in tumor growth and an increase in percent survival with combination therapy indicate that anti-TREM2 antibodies have additive or synergistic therapeutic effects with stimulatory checkpoint antibodies. Stimulatory checkpoint antibodies include agonistic/stimulatory antibodies against CD28, ICOS, CD137, CD27, CD40, and GITR.

### Example 22: Analysis of Additive Anti-Tumor Effect of Combination Therapy that Combines TREM2 Antibodies with Stimulatory Cytokines

Groups of 15 C57B16/NTac mice at 8 weeks (+/−2 weeks) of age are challenged subcutaneously with tumor cells as described in Example 19. Animals are anesthetized with isoflurane prior to implant. Starting at day 2, mice are injected intraperitoneally every 3 days for 4 doses with 200 ug anti-TREM2 antibodies alone or in combination with stimulatory cytokines (e.g., IL-12, IFN-a). Tumor growth is monitored with a caliper biweekly to measure tumor growth starting at day 4. The endpoint of the experiment is a tumor volume of 2000 mm³ or 60 days. Tumor growth and percent survival are the outcome measures. A decrease in tumor growth and an increase in percent survival with combination therapy indicate that anti-TREM2 antibodies have additive or synergistic therapeutic effects with immune-stimulatory cytokines. Stimulatory cytokines include IFN-α/b, IL-2, IL-12, IL-18, GM-CSF, and G-CSF.

### Example 23: Characterization of the Therapeutic Use of Agonistic TREM2 and/or TREM2 Bispecific Antibodies in a Model of Inflammatory Diseases

The therapeutic utility of agonistic anti-TREM2, and/or TREM2 bispecific antibodies is tested in a model of inflammatory diseases. For example rheumatoid arthritis or in an established model of another inflammatory disease (Mizoguchi (2012) Prog Mol Biol Transl Sci., 105:263-320; and Asquith et al., (2009) Eur J Immunol. 39:2040-4).

### Example 24: In Vivo Protection from EAE and Cuprizone in a Whole Animal

Adult 7-9 week-old female C57BL/6 mice (obtained from Charles River Laboratories) are injected in the tail base bilaterally with 200 μl of an innoculum containing 100 ng of myelin oligodendrocyte glycoprotein peptide 35-55 (amino acids MEVGWYRSPFSRVVHLYRNGK (SEQ ID NO: 885); Seqlab) and 1 mg of Mycobacterium tuberculosis H37 Ra (Difco) in incomplete Freund adjuvant (Difco). Pertussis toxin (200 ng; List Bio-logical Laboratories) is injected at day 0 and at day 2 after immunization. Clinical signs are scored as follows: 0, no clinical signs; 1, complete limp tail; 2, complete limp tail and abnormal gait; 3, one hind-limb paraparesis; 4, complete hindlimb paraparesis; and 5, fore- and hind-limb paralysis or moribund. Only mice having disease onset (clinical score of 1 or more) at day 14 are used for experiments. Agonistic anti-TREM2, and/or TREM2 bispecific antibodies are injected intraperitoneally or intravenously in EAE-diseased mice at the day of the first clinical symptoms or at any other desired time (PLoS Med (2007) 4(4): e124).

Young or aged wild-type (WT) mice are fed a standard diet (Harlan) containing 0.2% cuprizone (CPZ) powdered oxalic bis(cyclohexylidenehydrazide) (Sigma-Aldrich) for 4, 6 or 12 weeks. For Histological and immunohistochemical analyses brains are removed after mouse perfusion with 4% paraformaldehyde (PFA), fixed in 4% PFA for 24 h, followed by immersion in 30% sucrose for 24-48 h. To evaluate myelin integrity and damage, as well as cell proliferation and inflammation sections or mouse brain are stained with anti-MBP (1:100; Abcam, ab7349), -dMBP (1:2000; Millipore, ab5864), -β APP (1:100; Invitrogen, 51-2700), -SMI-31 (1:1000; Covance, smi-31R), -Ibal (1:600; Wako, 019-19741), -BrdU (1:250; Abcam, 7E5893), -GFAP (1:200; Invitrogen, 13-0300), -iNOS (1:100; BD Pharmingen, 610329), -LPL(1:400, from Dr. G. Olivecrona) and -MHC II (1:100; BD Pharmingen, 553549). For behavioral effects of the antibodies, mice are analyzed for locomotor activity using transparent polystyrene enclosures and computerized photobeam instrumentation. General activity variables (total ambulations, vertical rearings), along with indices of emotionality including time spent, distance traveled and entries, are analyzed. A battery of sensorimotor tests is performed to assess balance (ledge and platform), strength (inverted screen), coordination (pole and inclined screens) and initiation of movement (walking initiation). Motor coordination and balance are studied using a rotarod protocol (Cantoni et al., Acta Neuropaehol c2015)129(3):429-47).

### Example 25: Characterization of the Therapeutic Use of Agonistic TREM2 and/or TREM2 Bispecific Antibodies in Established Animal Models of Traumatic Brain Injury

The therapeutic utility of agonistic anti-TREM2, and/or TREM2 bispecific antibodies is tested in established animal models of traumatic brain injury (Tanaka, Y et al. (2013) Neuroscience 231 49-60). For example, a model of traumatic brain injury that induces the activation of microglia and astrocytes is used. Eight or nine week-old male C57BL/6J WT mice or progranulin heterozygous mice are used (purchased from Charles River Laboratories or Jackson Laboratories). Mice are anesthetized by intraperitoneal administration of xylazine hydrochloride (8 mg/kg) and chloral hydrate (300 mg/kg) dissolved in sterile saline, and subsequently placed in a stereotaxic apparatus (Narishige, Tokyo, Japan). An incision is made in the scalp and the cranium is exposed. The periosteum is cleaned from the skull, a hole is drilled over the right cerebral hemisphere with a dental drill, and the duramater is removed with a needle tip. A stainless steel cannula, with a 0.5 mm outer diameter, is used to make a longitudinal stab wound in the right hemisphere. The cannula is positioned at 1.3 mm lateral to the midline, and 1 mm posterior to bregma, and introduced into the brain until the tip reaches a depth of 2 mm. The cannula is then shifted 2 mm caudally (bregma 3 mm), and then shifts back 2 mm rostrally to its initial position. Finally, the cannula is removed from the brain, and the scalp wound is sutured.

Alternatively, a modified weight-drop device is used (Chen, Y., et al., (1996) J. Neurotrauma 13, 557-568). Specifically, following isoflurane anesthesia, a midline longitudinal incision is made and the skull exposed. A Teflon-tipped cone (2-mm diameter) is placed 1-2 mm lateral to the midline in the midcoronal plane. The head is manually held in place, and a 95-g weight is dropped on the cone from a prefixed height, resulting in a focal injury to the left hemisphere. After recovery from anesthesia, the mice are returned to their home cages with postoperative care and free access to food and water. Sham controls received anesthesia and skin incision only. Mice are treated with Trem2 antibodies delivered by Intraperitoneal injection at a volume of 250 ul/mouse (calculated as 100 ul/10 gr Body weight) at concentration of antibodies ranging from 4 mg/ml to 0.5 mg/ml. Control IgG antibody is injected at a concentration of 4 mg/ml. Antibodies are injected at days −3 to the traumatic brain injury and then at days 1, 7, 14, 21, 28. Neurological score (NSS) is evaluated 1 hour after TBI (to define, and ensure similar severity of injury in all groups) and then at 24 hour, and days 3, 5, 7, and once weekly till the end of the follow-up (4 weeks). Cognitive functions are being tested at days 4, 16, 32 after injury) using the novel object recognition test.

The Neurological Severity Score (NSS), is performed as described (Beni-Adani, L. et al., (2001) J. Neurotrauma 25, 324-333; Tsenter, J. et al., (2008). J. Neurotrauma 25, 324-333). Specifically, NSS consists of 10 individual tasks, including open-field performance, beam walk, balance, and hemiparesis evaluations, which reflect motor function, alertness, and behavior. One point is given for failure to perform a task and 0 for success. The NSS at 1 h post-trauma reflects the initial severity of injury. Thus, the extent of recovery (delta NSS) is calculated as the difference between the initial NSS score at 1 h postinjury and at any subsequent time point.

The novel object recognition test (NORT) is a sensitive and reproducible test for measuring cognitive abnormalities in TBI. Mice are placed for 1 h habituation period in an open glass aquarium-like transparent box, each at a time, in a sound-isolated room. On the following day they are re-introduced in the box for 5 min with two identical clean plaster objects, placed in two different corners of the box to measure baseline activity. Four hours later, one of the objects is replaced with a new one of the same size and texture, and the mice were re-introduced for additional 5 min into the same cage to test for novel object recognition. The time spent by the mouse in object exploration was recorded manually by an operator blinded to the different treatments. The cumulative time spent at each of the objects was recorded. Exploration of an object is defined as directing the nose to the object at a distance of 2 cm and/or touching it with the nose. The percentage of the total exploration time that the animal spent investigating the new object out of total exploration time is the measure of recognition memory. At baseline, the mouse spent about equal times at both objects since both are novel for him. At test, the cognitively healthy mice will identify the new object as “new”, remembering the old one, and therefore will spend more time exploring the new object (˜70-75% of the time). TBI leads to impaired memory, thus shorter (than normal) percent of the time exploring the new object. Some spontaneous recovery from TBI does occur, and could lead to TBI mice spending 60-65% of the time at the novel object. For statistical analyses, commercially available computer software (SigmaStat 2.03, Systat Software, San Jose, Calif., USA) can be used. Treatments are the independent variables and the outcomes of the TBI parameters are the dependent variables. Significance for NSS and NORT experimental series are tested using two-way ANOVA for repeated measures, and post hoc Fisher's PLSD test. Data are expressed as mean±s.e.m.

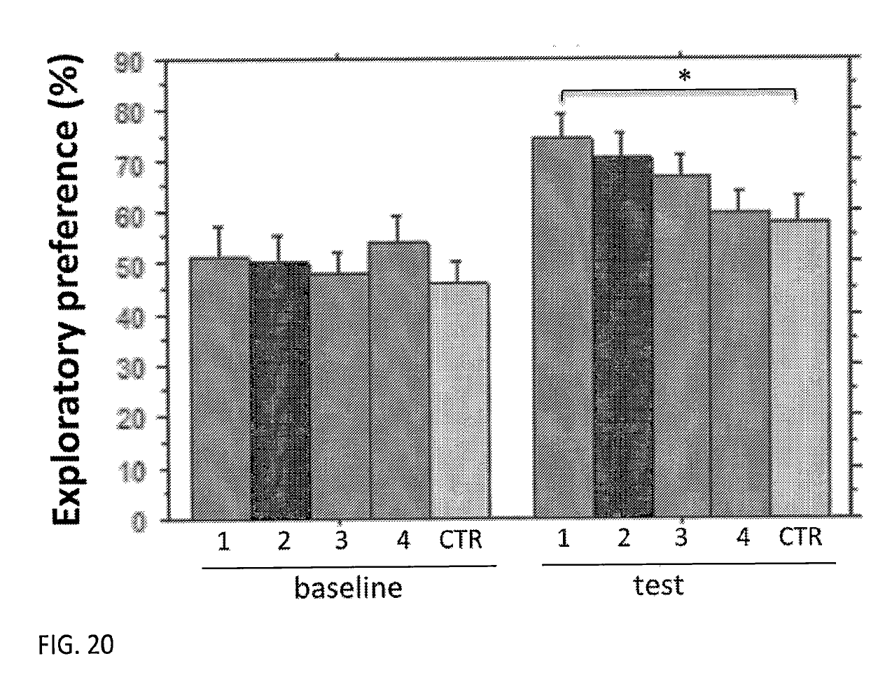

As shown in FIG. 20, a dose dependent improvement in cognitive function was observed in mice with traumatic brain injury that were treated with different doses of anti-TREM2 antibody 7E5. Cognitive function was assessed with the NORT test. Treatment groups included: 1=40 mg/Kg 7E5; 2=20 mg/Kg 7E5; 3=10 mg/Kg 7E5; 4=5 mg/Kg 7E5 and CTR=40 mg/Kg isotype control antibody mIgGl. The NORT test was performed at day 32 after injury. Bar graphs represent the percentage of time that mice spent investigating the new object. “Baseline” bar graphs represent the time spent exploring two identical objects, which is similar regardless of the treatment that the mice have received. “Test” bar graphs represent the time spent exploring a new object. The post hoc Fisher's PLSD test was used for statistical analysis *=Pval<0.05.

Mice with traumatic brain injury that were treated with the control antibody spent only 57.4±5.3% exploring the novel object, which is indicative of highly unpaired cognitive function (FIG. 20). By contrast, mice treated with the highest dose of anti-TREM2 antibody 7E5 spent 73.9±5.4% of the time exploring the novel object, which is close to normal cognitive function, indicating almost full recovery (FIG. 20).

### Example 26: Characterization of Therapeutic Use of Agonistic TREM2 and/or TREM2 Bispecific Antibodies in a Model of Neuro-Inflammation and Neuron Loss Following Toxin-Induced Injury

The therapeutic utility of agonistic anti-TREM2, and/or TREM2 bispecific antibodies is tested in a model of neuro-inflammation and neuron loss following toxin-induced injury (Martens, L H et al., (2012) The Journal of Clinical Investigation, 122, 3955). Three-month-old mice are treated with 4 intraperitoneal injections of MPTP (1-methyl-4-phenyl-1,2,3,6-tetrahydropyridine) per day for 2 days (4 μg/g body weight) (Sigma-Aldrich) or PBS. Mice are treated with agonistic anti-TREM2, and/or TREM2 bispecific antibodies according to standard protocols and then analyzed using Stereological counting to quantify dopamine neurons and microglia in the substantia nigra pars compacta (SNpc), as described.

### Example 27: Enhancement of the Ability of BMDCs to Induce Antigen-Specific T-Cell Proliferation by Agonistic and/or Bispecific TREM2 Antibodies

It is believed that agonistic anti-TREM2, and/or TREM2 bispecific antibodies may increase ability of bone marrow-derived dendritic cells (BMDC) to express the markers CD83 and CD86 and then to induce antigen-specific T-cell proliferation. To determine if TREM2 antibodies induce expression of the cell surface markers CD83 and CD86 on dendritic cells, antibodies are plated overnight at 4° C. in 12 well plates at 2 or 5 μg/ml in PBS. Wells are washed 3× with PBS the next day and day 5 immature human DCs are harvested and plated at 1 million cells per well and incubated at 37C, 5% CO₂ in the absence of cytokine. FACS analysis of CD86, CD83 and CD11c (BD Biosciences) is performed on a BD FACS Canto 48 hours later. Data analysis was performed with FlowJo (TreeStar) software version 10.0.7. Alternatively, day 5 immature human dendritic cells are plated at 100,000 cells per well in a U-bottom non-TC treated 96 well plate in media without cytokine. Antibodies are added at 5 μg/ml with or without LPS-removed anti-human secondary antibody (Jackson ImmunoResearch) at 20 μg/ml. FACS analysis for CD86, CD83, and CD11c (BD Biosciences) is performed 48 hrs post antibody addition as previously described. Ovalbumin (OVA)-specific T-cell response induced by BMDCs can be determined by CFSE dilution. BMDCs are isolated by MACS after 6 days of culture and plated at 1×10⁴ cells per well of a round bottom 96 well plate with OVA (2 or 0.5 mg/mL) and CpG DNA (100 or 25 nM) in the presence of GM-CSF (10 ng/mL) for 4 h. CD4 T-cells from the spleen and lymph nodes of OT-II transgenic mice are isolated by using Dynal Mouse CD4 Negative Isolation Kit (Invitrogen) and stained with CFSE (final 0.8 mM). After 4 h of DC culture, 1×10 CFSE-labeled CD4 OT-II T-cells are added into each well and incubated for 72 h. After culturing, cells are stained with an anti-CD4 monoclonal antibody and flow cytometry is performed to detect CFSE dilution of gated CD4 OT-II T-cells. Data analysis to calculate the percentage of divided and division index is performed by Flowjo software (Treestar) (Eur. J. Immunol. 2012. 42: 176-185).

Alternatively, Day 5 immature dendritic cells (CD14⁻CD11c⁺ LIN⁻) are plated in 12 well dishes coated the previous day with 2_{K}g/ml antibody. Plates are washed 3 times with PBS before addition of T cells. CD4⁺ T cells from nonautologous donors were isolated and labeled with CFSE before addition to DCs in ratio of 1:10. CD3/CD28 Dynal beads serve as a positive control. Day 5 post co-culture cells are analyzed by flow cytometry on a BD FACSCanto II for CFSE dilution. Percent CFSE^{hi} compared to CFSE^{lo} cells are calculated for each condition with FlowJo (TreeStar).

### Example 28: TREM2 Antibodies Induce the Expression of CD83 and CD86 on Human Dendritic Cells (DCs) and Induce T Cell Proliferation

To evaluate the ability of anti-TREM2 antibodies to modify expression of CD83 and CD86, both plate-bound and soluble antibodies were incubated with dendritic cells (DCs), and the expression of CD83, CD86, CCR7, and phosphorylated ERK were measured. To evaluate the ability of anti-TREM2 antibodies to modulate T cell proliferation, DCs were incubated with T cells and anti-TREM2 antibodies, and the level of T cell proliferation is measured. Antibodies are plated overnight at 4C in 12 well plates at 2 or 5 ug/ml in PBS. Wells are washed 3× with PBS the next day. On day 5, immature human DCs are harvested and plated at 1 million cells per well and incubated at 37C, 5% CO₂ in the absence of cytokine. FACS analysis of CD86, CD83, CD11c, HLA-DR, and LIN (BD Biosciences) are performed on a BD FACS Canto 48 hours later. Data analysis is performed with FlowJo (TreeStar) software version 10.0.7. Levels of CD83, CD86, and CCR7 are evaluated for CD11c+HLA-DR+LIN− cell populations. For intracellular ERK phosphorylation, cells are fixed with 1% formaldehyde, permeabilized with cytofix/cytoperm kit (BD), and intracellular Erk phopshorylation is determined with flow cytometry after staining with PE-ERK antibody (BD).

Alternatively, Day 5 immature human dendritic cells are plated at 100,000 cells per well in a U-bottom non-TC treated 96 well plate in media without cytokine. Antibodies are added at 5 ug/ml with or without LPS-removed anti-human secondary (Jackson ImmunoResearch) at 20 ug/ml. FACS analysis for CD86, CD83, CD11c, HLA-DR, and LIN (BD Biosciences) is performed 48 hrs post antibody addition as previously described. Additionally, Day 5 immature dendritic cells (CD14⁻ CD11c⁺ LIN⁻) are plated in 12 well dishes coated the previous day with Zug/ml antibody. Plates are washed 3 times with PBS before addition of T cells. CD4⁺ T cells from non-autologous donors were isolated and labeled with CFSE before addition to DCs in ratio of 1:10, 1:50, or 1:250. CD3/CD28 Dynal beads serve as a positive control. Day 5 post co-culture cells are analyzed by flow cytometry on a BD FACSCanto II for CFSE dilution. Percent CFSE^{hi} compared to CFSE^{lo} cells are calculated for each condition with FlowJo (TreeStar).

### Example 29: Normalization and Increase of Toll-Like Receptor (TLR) Responses in Macrophages by Agonistic and/or Bispecific TREM2, Antibodies

To evaluate the ability of anti-TREM2 antibodies to modify TLR response, Bone marrow-derived macrophages (BMDM) or primary peritoneal macrophage responses are altered to TLR signaling by deficiency of TREM2 (Turnbull, I R et al., J Immunol 2006; 177:3520-3524). It is believed that agonistic anti-TREM2, and/or TREM2 bispecific antibodies may increase or normalize TLR responses in macrophages. To elicit primary macrophages, mice are treated with 1.5 ml of 2% thioglycollate medium by intraperitoneal injection, and cells are then isolated by peritoneal lavage. To generate BMDM, total bone marrow is cultured in DMEM supplemented with 10% bovine calf serum, 5% horse serum, and 6 ng/ml recombinant human CSF-1 (R&D Systems). Cells are cultured for 5-6 days, and adherent cells are detached with 1m MEDTA in PBS. Cells are stained with commercially available antibodies: anti-CD11b, anti-CD40, anti-GR1 (BD Pharmingen), and F4/80 (Caltag Laboratories). BMDM are re-plated and allowed to adhere for 4 h at 37° C., and then TLR agonists, such as LPS (Salmonella abortus equi), zymosan (Saccharomyces cerevisiae), and CpG 1826 DNA (purchased from e.g., Sigma-Aldrich) are added. Cell culture supernatant is collected 24 h after stimulation and the levels of IFN-α4, IFN-b, IL-6, IL-12 p70, and TNF Cytokine concentrations in the culture supernatants are determined using mouse IFN-α4, IFN-b, IL-6, IL-12 p70, TNF, and IL-10 ELISA kits (eBioscience) and VeriKine Mouse IFN-b ELISA kit (PBL interferon source) according to manufacturer's protocol. Alternatively Cytometric Bead Array for human or mouse cytokines (BD Biosciences), or a V-PLEX Human or mouse Cytokine system with the Meso scale discovery System can be used. Alternatively, to analyze cytokines secretion BM derived macrophages of the indicated genotype are harvested at day 5 and plated on 96-well plate at 10⁵ cells/well. Cells are then stimulated with the indicated concentration of LPS or zymosan. 24 hours later, cell culture supernatants are harvested and analyzed by FACS for the presence of inflammatory cytokines (IL-12, IL-10, IFN-γ, TNFa, IL-6, MCP-1) using a Cytometric Bead Array kit (BD, following manufacturer's instructions). Cells are also analyzed by FACS to assess viability (DAPI) and expression of surface markers (CD11b, CD86).

### Example 30: TREM2 Increases Secretion of Inflammatory Cytokines from Macrophages

Bone marrow-derived macrophages (BMDM) or primary peritoneal macrophage responses possess altered TLR signaling when deficient in TREM2 (Turnbull, I R et al., J Immunol 2006; 177:3520-3524). In order to determine whether TREM2 antibodies induce changes in inflammatory cytokine production, mouse wild-type (WT) and TREM2 knock-out mice (KO) or TREM2 heterozygous mice (HETS) are cultured with the antibodies alone or with the antibodies in combinations with non-saturating levels of TLR stimulators and the level of cytokines are measured after 24-48h. To generate BMDM, total bone marrow from wild-type (WT), was cultured in RPMI supplemented with 10% bovine calf serum, 5% horse serum, and 50 ng/ml recombinant mouse CSF-1 (R&D Systems). Cells are cultured for 5 days, and adherent cells are detached with 1 mM EDTA in PBS. BMDM are plated on 96-well plates at 10⁵ cells/well and allowed to adhere for 4 h at 37° C. Cells are then exposed to antibodies alone, stimulated with TLR agonists LPS (Salmonella abortus equi) or zymosan (Saccharomyces cerevisiae) at concentrations ranging from 0.01-100 ng/ml (LPS) or 0.01-100 □g/ml (zymosan) alone or stimulated with LPS or zymosan in combination with Trem2 antibodies. Alternatively, macrophages isolated from WT and KO mice are cultured in the presence of 10 ng/ml of the cytokine IL-4 or 50 ng/ml of IFN-gamma with or without Trem2 antibodies. Cell culture supernatant was collected 24 or 48 h after stimulation and the levels of TNFa, IL-6, IL-10, and MCP-1 cytokines were measured by using Cytometric Bead Array Mouse Inflammation Kit (BD) according to manufacturer's protocol.

### Example 31: In Vivo Effect of Anti-TREM2 Antibodies on Inflammatory Cytokine Production

Mouse macrophages were stimulated directly in vivo on Brewer's Thioglycollate-induced peritoneal macrophages and then the concentration of cytokines in the peritoneal cavity was measured. In order to determine whether TREM2 antibodies induce changes in inflammatory cytokine production, wild-type (WT) mice and TREM2 knock-out mice (KO) were injected intraperitoneally with 3 ml of 3% Brewer's Thioglycollate at day 0. At day 3, mice were injected intraperitoneally with anti-TREM2 antibody 7E5 or isotype control antibody (mIgG1) for 15 min or 24 hr. Peritoneal cells were then harvested by peritoneal lavage using 4 ml saline solution and washed with PBS. Levels of TNFa and MCP-1/CCL2 cytokines were measured by using Cytometric Bead Array Mouse Inflammation Kit (BD) according to manufacturer's protocol.

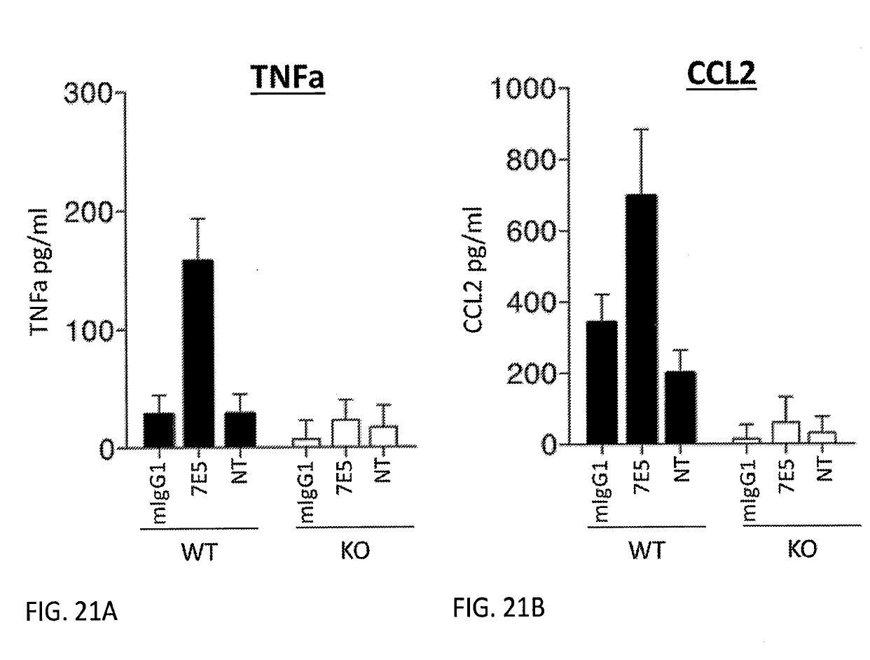

As shown in FIGS. 21A and 21B, when WT mice were treated with the 7E5 antibody the level of TNFa and CCL2 increased, as compared to mice treated with the isotype control antibody. In particular, the concentration of TNFa increased about 6-fold in mice treated with antibody 7E5, as compared to isotype control treated mice (FIG. 21A). The concentration of CCL2 increased ˜2 fold in mice treated with antibody 7E5, as compared to isotype control treated mice (FIG. 21B). The 7E5 antibody does not have an effect when administered to KO mice (FIGS. 21A and 21B). The results indicate that the induction of TNFa and CCL2 is specific to TREM2.

### Example 32: Inhibition of the Anti-Inflammatory Cytokine IL-10 in Bone Marrow-Derived Myeloid Precursor Cells by Agonistic and/or Bispecific TREM2 Antibodies

It is believed that bone marrow-derived myeloid precursor cells may show a decrease in the anti-inflammatory cytokine IL-10 following treatment with agonistic anti-TREM2, and/or TREM2 bispecific antibodies and stimulation with 100 ng/ml LPS (Sigma), by co-culturing with apoptotic cells, or by a similar stimulus. Isolation of bone marrow-derived myeloid precursor cells is performed as follows. Bone marrow cells are isolated from adult 6-8 week-old female C57BL/6 mice (Charles River, Sulzfeld, Germany) and from TREM2 deficient mice (KOMP repository) from the medullary cavities of the tibia and femur of the hind limbs. Removal of erythrocytes is performed by lysis with hypotonic solution. Cells are cultured in DMEM medium (Invitrogen) containing 10% fetal calf serum (Pan Biotech) and 10 ng/ml of GM-CSF (R&D Systems) in 75 cm² culture flasks (Greiner Bio-One). After 24 h, non-adherent cells are collected and re-seeded in fresh 75 cm² culture flasks. Medium is changed after 5 d and cells are cultured for an additional 10-11 d. Cells are cultured in the presence or absence of Trem2 antibodies, supernatant is collected after 24 h, and the level of IL-10 released from the cells is determined by IL-10 ELISA according to manufacturer's instructions (QuantikineM mouse IL-10, R&D Systems) (JEM (2005), 201; 647-657; and PLoS Medicine (2004), 4|Issue 4|e124).

### Example 33: Induction of Phagocytosis in Cells from the Myeloid Lineage by Agonistic and/or Bispecific TREM2 Antibodies

It is believed that agonistic anti-TREM2 and/or TREM2 bispecific antibodies may induce phagocytosis of apoptotic neurons, nerve tissue debris, non-nerve tissue debris, bacteria, other foreign bodies, and disease-causing proteins, optionally, such as A beta peptide, alpha synuclein protein, Tau protein, TDP-43 protein, prion protein, huntingtin protein, RAN, translation Products antigen, including the DiPeptide Repeats, (DPRs peptides) composed of glycine-alanine (GA), glycine-proline (GP), glycine-arginine (GR), proline-alanine (PA), or proline-arginine (PR) in cells from the myeloid lineage, such as monocytes, Dendritic cells macrophages and microglia. The bispecific antibodies may be antibodies that recognize the TREM2 antigen and a second antigen that includes, without limitation, A beta peptide, antigen or an alpha synuclein protein antigen or, Tau protein antigen or, TDP-43 protein antigen or, prion protein antigen or, huntingtin protein antigen, or RAN, translation Products antigen, including the DiPeptide Repeats, (DPRs peptides) composed of glycine-alanine (GA), glycine-proline (GP), glycine-arginine (GR), proline-alanine (PA), or proline-arginine (PR). Monocytes are isolated from peripheral blood that is collected from adult C57BL/6 mice. Hypotonic lysis buffer depletes erythrocytes. Cells are plated on culture dishes in RPMI medium (Invitrogen) containing 10% fetal calf serum (Pan Biotech). Cells are cultured for several hours at 37° C. in 10% CO₂. After trypsinization, adherent cells are collected and used for phagocytosis experiments.

Microglial cells are prepared from the brains of post-natal day 3 to 5 (P3 to P5) C57BL/6 mice. In brief, meninges are removed mechanically, and the cells are dissociated by trituration and cultured in basal medium (BME; GIBCO BRL) supplemented with 10% FCS (PAN Biotech GmbH), 1% glucose (Sigma-Aldrich), 1% L-glutamine (GIBCO BRL), and 1% penicillin/streptomycin (GIBCO BRL), for 14 d to form a confluent glial monolayer. To collect microglial cells, the cultures are shaken on a rotary shaker (200 rpm) for 2 h. The attached astrocytes are used for immunohistochemistry. The detached microglial cells are seeded in normal culture dishes for 1 h, and then all non-adherent cells are removed and discarded. Purity of the isolated microglial cells is about 95% as determined by flow cytometry analysis with antibody directed against CD11b (BD Biosciences). Microglial cells are cultured in basal medium as previously described (Hickman S E et al., J Neurosci. 2008 Aug. 13; 28(33):8354-60; and Microglia Methods and Protocols Vol. 1041). Oligodendrocytes (i.e., neurons) and neuron-enriched cells are prepared from the brain of C57BL/6 mouse embryos (E15-16). In brief, brain tissue is isolated and mechanically dispersed and seeded in culture dishes pre-coated with 0.01 mg/ml poly-L-ornithin (Sigma-Aldrich) and 10 μg/ml laminin (Sigma-Aldrich). Cells are cultured in neuronal condition medium (BME; GIBCO BRL) supplemented with 2% B-27 supplement (GIBCO BRL), 1% glucose (Sigma-Aldrich), and 1% FCS (PAN Biotech GmbH). Cells are cultured for 5-10 d to obtain morphologically mature oligodendrocytes.

To conduct phagocytosis assays microglia, macrophages or dendritic cells are cultured with apoptotic neurons, nerve tissue debris, non-nerve tissue debris, bacteria, other foreign bodies, and disease-causing proteins. Neurons are cultured for 5-10 d, and okadaic acid is then added at the final concentration of 30 nM for 3 h to induce apoptosis. Neuronal cell membranes are labeled with CellTracker CM-DiI membrane dye (Molecular Probes). After incubation, apoptotic neurons or other targets of phagocytosis are washed two times and added to the transduced microglial culture at an effector/target ratio of 1:20. At 1 and 24 h after addition of apoptotic neurons, the number of microglia having phagocytosed neuronal cell membranes is counted under a confocal fluorescence microscope (Leica). Apoptotic cells are counted in three different areas at a magnification of 60. The amount of phagocytosis is confirmed by flow cytometry. Moreover, 24, 48, or 72 h after the addition of apoptotic neurons, cells are collected and used for RT-PCR of cytokines. To conduct microsphere bead or bacterial phagocytosis assay, microglia, macrophages or dendritic cells are treated with anti-TREM2 agonistic antibodies. After 24 h, 1.00 μm of red fluorescent microsphere beads (Fluoresbrite Polychromatic Red Microspheres; Polysciences Inc.) or, fluorescent labeled bacteria are added for 1 h. Phagocytosis of microsphere beads or, fluorescent labeled bacteria, by microglia is analyzed by fluorescence microscopy. Furthermore, microglia are collected from the culture plates and analyzed by flow cytometry. The percentage of microglia having phagocytosed beads is determined. To conduct amyloid phagocytosis assay, HiLyteFluor™ 647 (Anaspec)-Abeta-(1-40) is resuspended in Tris/EDTA (pH 8.2) at 20 mM and then incubated in the dark for 3 d at 37° C. to promote aggregation. Microglial, macrophages or dendritic cells are pretreated in low serum (0.5% FBS supplemented with insulin), LPS (50 ng/ml), IFNc (100 units/ml), and anti-TREM2 agonistic antibodies for 24 h prior to the addition of aggregated fluorescently labeled a beta peptide. Amyloid phagocytosis and surface expression of TREM2 are determined by flow cytometric analysis 5 h post-addition of 100 nM aggregated HiLyteFluor™ 647-Ab-(1-40) (ASN NEURO (2010) 2(3): 157-170). Phagocytosis of other disease-causing proteins is conducted in a similar manner.

### Example 34: Induction of CCR7 and Migration Toward CCL19 and CCL21 in Microglia, Macrophages, and Dendritic Cells by Agonistic TREM2, or TREM2 Bispecific Antibodies

It is believed that anti-TREM2, and/or TREM2/bispecific antibodies may induce CCR7 and migration toward CCL19 and CCL21 in microglial cells, macrophages, and dendritic cells. Microglial, macrophages or dendritic cells are either cultured with agonistic anti-TREM2, and/or TREM2/DAP12 bispecific antibodies, or with a control antibody. Cells are collected after 72 h, immuno-labeled with CCR7 specific anti-bodies, and analyzed by flow cytometry. To determine any functional consequences of increased CCR7 expression, a chemotaxis assay is performed. Microglia, macrophages or dendritic cells are stimulated via TREM2 with the agonistic anti-TREM2, and/or TREM2/DAP12 bispecific antibodies and placed in a two-chamber system. The number of microglial cells migrating toward the chemokine ligands CCL19 and CCL21 is quantified (JEM (2005), 201, 647-657). For the chemotaxis assay, microglial, macrophages or dendritic cells are exposed to the agonistic anti-TREM2 or TREM2/bispecific antibodies and treated with 1 μg/ml LPS. Microglia, macrophages or dendritic cells are transferred into the upper chamber of a transwell system (3 μm pore filter; Millipore) containing 450 μl medium with 100 ng/ml CCL19 or CCL21 (both from PeproTech) in the lower chamber. After a 1 h incubation period, the number of microglial macrophages or dendritic cells that have migrated to the lower chamber is counted in three independent areas by microscopy (JEM (2005), 201, 647-657).

### Example 35: Induction of F-Actin in Microglia, Macrophages, and Dendritic Cells by Agonistic TREM2, and/or TREM2 Bispecific Antibodies

It is believed that agonistic anti-TREM2, or TREM2 bispecific antibodies may induce F-actin in microglial cells, macrophages, and dendritic cells. Microglia, macrophages or dendritic cells and other cells of interest that are transduced with TREM2 or that express TREM2 are added to culture plates and then exposed to agonistic anti-TREM2, and/or TREM2 bispecific antibodies, or a control antibody. Cells are fixed, blocked, and then stained with Alexa Fluor 546-conjugated phalloidin (Molecular Probes) after 1 h and F-actin is labeled with a fluorescence dye. Images are collected by confocal laser scanning microscopy with a 40× objective lens (Leica). (JEM (2005), 201, 647-657).

### Example 36: Induction of Osteoclast Production and Increased Rate of Osteoclastogenesis by Agonistic TREM2, DAP12, and/or TREM2/DAP12 Bispecific Antibodies

It is believed that agonistic anti-TREM2 and/or TREM2 bispecific antibodies may induce osteoclast production and increase the rate of osteoclastogenesis. RAW264.7 cells that make osteoclasts or bone marrow-derived monocyte/macrophage (BMM) precursor cells are maintained in RPMI-1640 medium (Mediatech), or another appropriate medium, supplemented with 10% FBS (Atlantic Biologics, Atlanta, Ga., USA) and penicillin-streptomycin-glutamine (Mediatech). TREM2B cDNA with a FLAG epitope added to the N terminus is inserted into the retroviral vector pMXpie upstream of an IRES, followed by an eGFP cDNA sequence. Cells are transfected with pMXpie-FLAG TREM2B, using Fugene 6 (Roche) according to manufacturer's protocol. Cells are selected in puromycin (Sigma) at 2 μg/ml. Stable puromycin-resistant clones are screened for anti-FLAG M2 monoclonal antibody (Sigma) binding by using flow cytometry, and then subcloned and maintained on puromycin selection media.

RAW264.7 cells expressing TREM2B are seeded in 96-well plates with 3000 cells/well in alpha-MEM medium supplemented with 10% FBS, penicillin-streptomycin-glutamine, 50 ng/ml RANKL, and 20 ng/ml M-CSF. The medium is changed every 3 days, exposed to anti-TREM2 agonistic antibodies and the number of multinucleated (at least three nuclei) TRACP⁺ osteoclasts are counted and scored by light microscopy. To determine complexity and size, osteoclasts are counted by number of nuclei (>10 or 3-10 nuclei). The surface area of osteoclasts is also measured by using Image J software (NIH). In addition, expression levels of osteoclasts genes are determined. Total RNA is extracted from osteoclastogenic cultures at different time points using TRIzol reagent (Invitrogen). After first-strand cDNA synthesis using a SuperScript III kit (Invitrogen), real-time quantitative PCR reactions are performed for Nfatcl, Acp5, Ctsk, Cater, and Ccndl. Relative quantification of target mRNA expression is calculated and normalized to the expression of cyclophilin and expressed as (mRNA of the target gene/mRNA of cyclophilin) 3×10⁶. (J. OF BONE AND MINERAL RESEARCH (2006), 21, 237-245; J Immunol 2012; 188:2612-2621).

Alternatively, BMM cells are seeded onto the plates in triplicate wells and treated with RANKL, M-CSF, and with an anti-TREM2, and/or TREM2 bispecific antibody, or an isotype-matched control monoclonal antibody. The medium is changed every 3 days until large multinucleated cells are visible. After 3 to 5 days in culture, cells are fixed with 3.7% formaldehyde in PBS for 10 min. Plates are then washed twice in PBS, incubated for 30 s in a solution of 50% acetone and 50% ethanol, and washed with PBS. Cells are stained for tartrate-resistant acid phosphatase (TRAP) with a kit from Sigma (product 435). Multinucleated (more than two nuclei), TRAP-positive cells are then counted by light microscopy, as described (e.g., Peng et al., (2010) Sci Signal., 3(122): ra38).

### Example 37: Characterization of the Therapeutic Use of Agonistic TREM2 and/or TREM2 Bispecific Antibodies in Animal Models of Aging, Seizures, Spinal Cord Injury, Retinal Dystrophy, Frontotemporal Dementia, and Alzheimer's Disease

The therapeutic utility of agonistic anti-TREM2, and/or TREM2 bispecific antibodies is tested in animal models for aging, seizures, spinal cord injury, retinal dystrophy, frontotemporal dementia, Huntington disease, Parkinson's disease amyotrophic lateral sclerosis and Alzheimer's disease, as previously described (e.g., Beattie, M S et al., (2002) Neuron 36, 375-386; Volosin, M et al., (2006) J. Neurosci. 26, 7756-7766; Nykjaer, A et al., (2005) Curr. Opin. Neurobiol. 15, 49-57; Jansen, P et al., (2007) Nat. Neurosci. 10, 1449-1457; Volosin, M et al., (2008) J. Neurosci. 28, 9870-9879; Fahnestock, M et al., (2001) Mol. Cell Neurosci. 18, 210-220; Nakamura, K et al., (2007) Cell Death. Differ. 14, 1552-1554; Yune, T et al., (2007) Brain Res. 1183, 32-42; Wei, Y et al., (2007) Neurosci. Lett. 429, 169-174; Provenzano, M J et al., (2008) Laryngoscope 118, 87-93; Nykjaer, A et al., (2004) Nature 427, 843-848; Harrington, A W et al., (2004) Proc. Natl. Acad. Sci. U.S.A. 101, 6226-6230; Teng, H K et al., (2005) J. Neurosci. 25, 5455-5463; Jansen, P et al., (2007) Nat. Neurosci. 10, 1449-1457; Volosin, M et al., (2008) J. Neurosci. 28, 9870-9879; Fan, Y J et al., (2008) Eur. J. Neurosci. 27, 2380-2390; Al-Shawi, R et al., (2008) Eur. J. Neurosci. 27, 2103-2114; and Yano, H et al., (2009) J. Neurosci. 29, 14790-14802).

### Example 38: Characterization of the Therapeutic Use of Agonistic TREM2 and/or TREM2 Bispecific Antibodies in Models of Atherosclerosis

The therapeutic utility of agonistic anti-TREM2 and/or TREM2 bispecific antibodies is tested in models of atherosclerosis, as previously described (e.g., Lance, A et al., (2011) Diabetes, 60, 2285; and Kjolby, M et al., (2012) Cell Metabolism 12, 213-223).

### Example 39: Characterization of the Therapeutic Use of Agonistic TREM2 and/or TREM2 Bispecific Antibodies in a Model of Infection

The therapeutic utility of agonistic anti-TREM2 and/or TREM2 bispecific antibodies is tested in a model of infection. For example, Listeria monocytogenes or other infection in normal mice can be used, as previously described (e.g., Yin, F et al., (2009) J. Exp. Med, 207, 117-128).

### Example 40: Screening for Anti-TREM2 and/or TREM2 Bispecific Antibodies that Induce Phosphorylation of TREM2, DAP12, SYk, ERK, and AKT, which Indicate Activation of the PI3K Pathway

Cells (J774, RAW 264.7, BMM cells, or osteoclasts) are removed from tissue culture dishes with PBS-EDTA, washed with PBS, and counted. J774 (40×10⁶) or RAW 264.7 cells (10×10⁶ BMM or osteoclasts) are incubated with an anti-TREM2 and/or TREM2 bispecific antibody or with an isotype-matched control antibody at 1 μg/10⁶ cells for 20 min on ice or under other conditions. Cells are lysed in ice-cold radioimmunoprecipitation assay (RIPA) buffer for 20 min followed by centrifugation at 16,000 g for 10 min at 4° C. to remove insoluble materials. The resulting supernatant is subjected to immunoprecipitation reactions with the indicated antibodies (DAP12, ERK, or AKT) and protein A- or protein G-agarose (Sigma). The beads are extensively washed with RIPA buffer and the proteins are separated by SDS-polyacrylamide gel electrophoresis (SDS-PAGE). The proteins are then transferred to nitrocellulose membranes by Western blotting, incubated with the appropriate antibodies (antibodies that specifically recognize the phosphorylated form of DAP12, ERK, or AKT) and visualized with the enhanced chemiluminescence (ECL) system (Pierce), as described (e.g., Peng et al., (2010) Sci Signal., 3(122): ra38).

### Example 41: Screening for Anti-TREM2, and/or TREM2 Bispecific Antibodies that Induce Calcium Flux

BMM cells are washed twice with HEPES-containing buffer [20 mM HEPES (pH 7.3), 120 mM NaCl, 1 mM CaCl, 1 mM MgCl, 5 mM KCl, glucose (1 mg/ml), bovine serum albumin (1 mg/ml)] followed by incubation in 0.05% Pluronic F-127 (Invitrogen) and 1 μM Indo-1 AM (Invitrogen) for 20 min at 37° C. Cells are washed twice with HEPES buffer and are then stimulated with an anti-TREM2 and/or TREM2 bispecific antibody (16 μg/ml) or with a control antibody (16 μg/ml) and monitored by spectrophotometer (PTL Photon Technology International). The Indo-1 fluorescence emission is converted to calcium (Ca²) according to manufacturer's instructions (e.g., Peng et al., (2010) Sci Signal., 3(122): ra38).

### Example 42: Screening for Anti-TREM2 and/or TREM2 Bispecific Antibodies that Promote Survival of Osteoclasts and/or Microglia

Murine Bone Marrow precursors are obtained by flushing tibial and femoral marrow cells with cold PBS. After one wash with PBS, erythrocytes are lysed using ACK Lysing Buffer (Lonza), washed twice with PBS and suspended at 0.5×10⁶ cells/ml in complete RPMI media (10% FCS, Pen/Strep, Gln, neAA) with the indicated amounts of 50 ng/ml M-CSF to make macrophages or 10 ng/ml GM-CSF. For M2-type macrophages, 10 ng/ml IL-4 is added to the cultured cells. For M1-type macrophages, 50 ng/ml IFN-γ is added. In some experiment LPS or zymosan is added to the cell culture at day 5, at a concentration of 1 μg/ml-0.01 ng/ml. Recombinant cytokines were purchased by Peprotech. To analyze viability of BM derived macrophages, cells of the indicated genotype are prepared as above and cultured in graded concentrations of MCSF. Cells are either plated at 10⁵/200 μl in a 96-well plate (for viability analysis using a luciferase based-assay) or at 0.5×10⁶/1 ml in a 6-well plate (for Trypan Blue exclusion cell count) in non-tissue culture treated plates. Media containing fresh M-CSF is added at day 3. At the indicated time points cells are gently detached from the plates with 3 mM EDTA and counted using a Burker chamber. In some experiments cells are also stained for FACS analysis using CD11b antibody and DAPI. Alternatively, cells are directly incubated with ToxGlo reagent (Promega) and luciferase activity is determined. In some experiments MCSF is withdrawn or not from the culture media at day 5 and cell viability is analyzed 36 hours later by FACS. Mature osteoclast cell cultures are differentiated in 24-well dishes with RANKL and M-CSF. After 4 days, complete medium is substituted with serum-free medium to induce apoptosis. Cells are treated with RANKL, PBS, and an anti-TREM2 and/or TREM2 bispecific antibody, or an isotype-matched control antibody, during the overnight serum starvation. Cells are fixed in 1% paraformaldehyde and stained with a TUNEL-based kit (Millipore Corporation) according to manufacturer's instructions. Apoptotic nuclei are counted with a Nikon TE2000-E microscope with 20× magnification. Results are expressed as the percentage of apoptotic cells relative to the total number of cells in six randomly selected fields of the two wells, as described (e.g., Peng et al., (2010) Sci Signal., 3(122): ra38). Similar assays are performed with primary microglial cells.

### Example 43: TREM2 Increases the Survival of Macrophages and Dendritic Cells

To evaluate the role of TREM2 in cell survival, wild-type (WT), TREM2 knock-out (KO), and TREM2 heterozygous (Het) macrophages and dendritic cells are cultured in the presence of Trem2 antibodies or fragment thereof and cell viability is determined.

Murine bone marrow precursor cells from TREM2 WT, Het, and KO mice are obtained by flushing tibial and femoral marrow cells with cold PBS. After one wash with PBS, erythrocytes are lysed using ACK Lysing Buffer (Lonza), washed twice with PBS and suspended at 0.5×10⁶ cells/ml in complete RPMI media (10% FCS, Pen/Strep, Gln, neAA) with the indicated amounts of 50 ng/ml M-CSF to produce macrophages, or 10 ng/ml GM-CSF to produce dendritic cells. For M2-type macrophages, 10 ng/ml IL-4 is added to the cultured cells. For M1-type macrophages, 50 ng/ml IFN-α is added. In some experiments LPS or zymosan is added to the cell culture at day 5 at a concentration range of 1 μg/ml-0.01 ng/ml. Recombinant cytokines are purchased from Peprotech. To analyze viability of bone marrow-derived macrophages, cells are prepared as above and cultured in MCSF. Cells are either plated at 10⁵/200 μl in a 96-well plate (for viability analysis using a luciferase based-assay) or at 0.5×10⁶/1 ml in a 6-well plate (for Trypan Blue exclusion cell count) in non-tissue culture treated plates. Media containing fresh M-CSF is added at day 3. At indicated time points cells are gently detached from the plates with 3 mM EDTA and counted using a Burker chamber. For FACS analysis of live cells, macrophages are cultured either in 50 ng/ml MCSF for 6 days (+MCSF) or in 50 ng/ml MCSF for 4 days before MCSF is removed for an additional 36 hrs (−MCSF). Cells are stained using CD11b antibody and DAPI. For luciferase viability assays, cell viability is measured at day 5 of culture in graded concentrations of growth factors GMCSF (dendritic cells), MCSF (M1 macrophages), or MCSF+IL-4 (M2 macrophages). Cells are directly incubated with ToxGlo reagent (Promega) and luciferase activity (luminescence) is determined. For FACS analysis of viable macrophages cultured in the presence of inflammatory mediators IFN-α, LPS, or zymosan, cells are collected at day 5 and stained using CD11b antibody and DAPI. All experiments are conducted in the presence or absence of Trem2 antibodies or control antibodies or fragments thereof. Alternatively, WT mice are injected with 40 mg/kg or another dose of TREM2 or control antibodies intraperitoneally (IP) followed by IP injections of 2-4 mg/kg LPS 12-24h latter. Cells are collected from the abdominal cavity 6 hours later and analyzed by FACS using the following markers; CD11b-PB; CD11c Pecy7; MHC-II APCcy7; Grl FITC; Ly6G PE; Amcyan live/dead cells.

### Example 44: Screening for Anti-TREM2 and/or TREM2 Bispecific Antibodies that Normalize TREM2/TYROBP-Dependent Changes in Gene Expression within the Immune/Microglia Regulatory Module

Microglial cells derived from mouse embryonic stem cells are genetically modified by lentiviral vectors to overexpress either full-length or a truncated version of Tyrobp that lacks both intracellular immunoreceptor tyrosine-based activation motif (ITAM) motifs. Microglia cells are also derived from mouse embryonic stem cells that are heterozygous for TREM2. To assess the genome-wide gene-expression changes in response to the perturbation of Tyrobp or TREM2, gene-expression data is derived from the RNA sequencing of mouse microglial macrophages or dendritic cells overexpressing: (1) vehicle, (2) full-length Tyrobp, or (3) dominant-negative truncated Tyrobp; or (4) overexpressing a knockdown construct for TREM2, such as SiRNA and cells which are heterozygous for TREM2 as well as from cells derived from TREM2 deficient mouse. Approximately 2,638 and 3,415 differentially expressed genes for the overexpression of full-length Tyrobp and truncated Tyrob are identified, respectively (Zhang et al., (2013) Cell 153, 707-720). Approximately 99% of the differentially expressed genes from the microglia overexpressing intact Tyrobp are downregulated compared to the control vehicle. For example, 658 genes, related to the vacuole/autophagy, as well as genes involved with RNA metabolism and cell-cycle mitosis are downregulated by active Tyrobp, but upregulated in cells expressing dominant-negative truncated Tyrobp. Conversely, some 2,856 genes for the vacuole/autophagy pathway and for mitochondrion are selectively upregulated in microglia expressing the dominant-negative truncated Tyrobp. Agonistic anti-TREM2, and/or TREM2 bispecific antibodies are screened for their ability to elicit gene expression profiles similar to that observed in normal microglial cells and in microglial cells overexpressing intact Tyrobp in cells that express dominant-negative truncated Tyrobp (Zhang et al., (2013) Cell 153, 707-720), in cells that express the knockdown construct for TREM2, or in cells that are heterozygous for TREM2. Antibodies that are capable of changing the gene expression network are selected.

### Example 45: Analysis of the Anti-Cancer Effect of TREM2 Antibodies

Groups of 10 C57B16/NTac mice at 8 weeks (+/−2 weeks) of age are challenged subcutaneously with tumor cells (e.g. 1×10⁵ to 1×10⁶ MC38, Lewis Lung, or B16 cells) suspended in 100 ul PBS. Animals are anesthetized with isoflurane prior to implant. Starting at day 2, groups of mice are injected intraperitoneally every 3 days for 4 doses with 200 ug of each of antagonistic anti-TREM2 antibodies, such as those described in Examples 38 and 40. Tumor growth is monitored with a caliper biweekly to measure tumor growth starting at day 4. The endpoint of the experiment is a tumor volume of 2000 mm³ or 60 days. Tumor growth and percent survival are the outcome measures. Reduced tumor take and growth rate, reduced number of tumor infiltrating immune suppressor macrophages, and increased effector T cell influx into the tumor indicate the anti-cancer effects of blocking anti-TREM2 antibodies.

### Example 46: Analysis of Additive Anti-Tumor Effect of Combination Therapy that Combines TREM2 Antibodies with Antibodies Against Inhibitory Checkpoint Proteins or Inhibitory Cytokines/Chemokines and their Receptors

Groups of 15 C57B16/NTac mice at 8 weeks (+/−2 weeks) of age are challenged subcutaneously with tumor cells. Animals are anesthetized with isoflurane prior to implant. Starting at day 2, mice are injected i.p. every 3 days for 4 doses with 200 ug anti-TREM2 antibodies alone or in combination with antibodies against checkpoint proteins (e.g. anti-PDL1 mAb clone 10F.9G2 and/or anti-CTLA-4 mAb clone UC10-4F10-11) at day 3, 6, and 9. Treatment groups include anti-TREM2; anti-CTLA-4; anti-PDL1; anti-TREM2+anti-CTLA-4; anti-TREM2+anti-PDL1; and isotype control. Tumor growth is monitored with a caliper biweekly to measure tumor growth starting at day 4. The endpoint of the experiment is a tumor volume of 2000 mm³ or 60 days. Tumor growth and % survival are the outcome measures. A decrease in tumor growth and an increase in % survival with combination therapy indicate that anti-TREM2 antibodies have additive or synergistic therapeutic effects with anti-checkpoint antibodies. Antagonistic antibodies against checkpoint molecules include antibodies against PDL1, PDL2, PD1, CTLA-4, B7-H3, B7-H4, HVEM, BTLA, KIR, GALS, TIM3, A2AR, LAG-3, and phosphatidylserine (PS). Antagonist antibodies against inhibitory cytokines include antibodies against CCL2, CSF-1, and IL-2.

### Example 47: Analysis of Additive Anti-Tumor Effect of Combination Therapy that Combines TREM2 Antibodies with Antibodies that Activate Stimulatory Checkpoint Proteins

Groups of 15 C57B16/NTac mice at 8 weeks (+/−2 weeks) of age are challenged subcutaneously with tumor cells. Animals are anesthetized with isoflurane prior to implant. Starting at day 2, mice are injected intraperitoneally every 3 days for 4 doses with 200 ug anti-TREM2 antibodies alone or in combination with agonistic antibodies that activate stimulatory checkpoint proteins (e.g., OX40 or ICOS mAb) at day 3, 6, and 9. Tumor growth is monitored with a caliper biweekly to measure tumor growth starting at day 4. The endpoint of the experiment is a tumor volume of 2000 mm³ or 60 days. Tumor growth and percent survival are the outcome measures. A decrease in tumor growth and an increase in % survival with combination therapy indicate that anti-TREM2 antibodies have additive or synergistic therapeutic effects with stimulatory checkpoint antibodies. Stimulatory checkpoint antibodies include agonistic/stimulatory antibodies against CD28, ICOS, CD137, CD27, CD40, and GITR.

### Example 48: Analysis of Anti-Stroke Effect of TREM2 Antibodies

Transient occlusion of the middle cerebral artery (MCAO)—a model that closely resembles human stroke is used to induce cerebral infarction in mice. Monofilament (70SPRe, Doccol Corp, USA) is introduced into the internal carotid artery through an incision of the right common carotid artery. The middle cerebral artery is occluded for 30 minutes with a range of reperfusion times (6 h, 12 h, 24 h, 2 d, 7 d and 28 d). The effect of surgery is controlled using sham animals at 12 h and at 7 d. Sham animals undergo the same surgical procedure without occlusion of the middle cerebral artery. MCAO animals treated with agonistic anti-TREM2 antibodies or control antibodies are tested for infarct volumetry, acute inflammatory response (12 h reperfusion), transcription of pro-inflammatory cytokines TNFa, IL-1α, and IL-lb, microglial activity (CD68, Ibal), transcription of chemokines CCL2 (MCP1), CCL3 (MIPla and the chemokine receptor CX3CR1 and invasion of CD3-positive T-cells (Sieber et al. (2013) PLoS ONE 8(1): e52982. doi:10.1371/journal.pone.0052982.).

### Example 49: Analysis of Anti-Alzheimer's Disease Effect of Anti-TREM2 Antibodies

To evaluate the ability of anti-TREM2 antibodies to delay, prevent, or reverse the development of Alzheimer's disease (AD), 5XFAD mice are used. 5XFAD mice overexpress mutant human APP (695) with the Swedish (K670N, M671L), Florida (1716V), and London (V717I) familial Alzheimer's disease (FAD) mutations, along with human PS1 harboring two FAD mutations, M146L and L286V. Both transgenes are regulated by the mouse Thyl promoter to drive over expression on the brain and recapitulate major features of AD. Mice treated with the agonistic anti-TREM2 antibodies or with control antibodies are tested for A beta plaque load with immunohistochemistry and by ELISA of tissue extracts. They are further tested for the number of microglia in the brain, and for reduction in cognitive deficit using Morris Water maze, a spatial learning and memory task, Radial Arm Water Maze, a spatial learning and memory task, Y Maze (quantifies spontaneous alternation as a measure of spatial cognition), novelty preference in in an open field, operant learning to assess learning and memory, and fear conditioning (mousebiology.org website; Wang et al., (2015) Cell, pii: S0092-8674(15)00127-0).

### Example 50: Analysis of the Protective Effect of TREM2 Antibodies in Respiratory Tract Infections

To evaluate the ability of TREM2 antibodies to delay, prevent, or treat bacterial respiratory tract infections, a preclinical mouse model involving challenge of C57B16 mice with Streptococcus pneumoniae is used. This model involves intranasal (i.n.) administration of 105 CFU S. pneumoniae serotype 3 (ATCC 6303) as described (see, e.g., Sharif 0 et al, 2014 PLoS Pathog. 2014 June; 10(6): e1004167; and Schabbauer G et al, 2010 J Immunol 185: 468-476). In this model ˜90% WT C57B16 mice succumb to infection within 6 days post infection. Ten to fifteen mice/group are challenged with S. pneumoniae and concomitantly are treated with antagonist anti-TREM2 antibodies every other day starting from day 0. The first dose of anti-TREM2 antibodies is administered 3 hours prior to challenge with S. pneumonia. Mice are monitored daily for 15 days to check for death events. % of mice surviving bacteria challenge is determined. In separate experiments, count of bacterial load and cytokine expression in the blood and in the lungs is also determined. 24 or 48 hours after infection blood is collected in EDTA-containing tubes and plated on agar plates to enumerate bacterial CFU in the plasma. Plasma is stored at −20° C. for cytokine analysis by ELISA. Lungs are harvested, homogenized and plated on agar plates to enumerate bacterial CFU, or incubated for 30 min in lysis buffer and supernatants analyzed for cytokine measurements. In separate experiments, lungs are collected 40 hours post bacterial infection, fixed in 10% formalin, and embedded in paraffin for H&E pathology analysis.

### Example 51: Analysis of the Protective Effect of TREM2 Antibodies in Sepsis

To evaluate the ability of TREM2 antibodies to delay, prevent, or treat sepsis, a preclinical mouse model involving systemic challenge of C57B16 mice with LPS is used. This model involves intraperitoneal (i.p.) administration of 37 mg/ml LPS as described (see, e.g., Gawish R et al, 2014 FASEB J). In this model >95% WT C57B16 mice succumb infection within 40 hours post LPS injection. Cohorts of mice are challenged with LPS and concomitantly are treated with antagonist anti-TREM2 antibodies every day starting from day 0. The first dose of anti-TREM2 antibodies is administered 3 hours prior to challenge with LPS. Mice are monitored every ˜4 hours during daytime, to check for death events. Percentage of mice surviving LPS challenge is determined.

In separate experiments, peritoneal lavage fluid (PLF) is collected. Supernatants are stored at −20° C. for cytokine analysis by ELISA; pelleted cells are counted to quantify inflammatory cells recruited in the peritoneal cavity. Similar studies can be conducted to test the efficacy of TREM2 antibodies in other models of infection (see, e.g., Sun et al., (2013) Invest Ophthalmol Vis Sci. 17; 54(5):3451-62).

### Example 52: Analysis of the Protective Effect of TREM2 Antibodies in Acute and Chronic Colitis

To evaluate the ability of anti-TREM2 antibodies to delay, prevent, or treat colitis, preclinical mouse models of acute or chronic colitis are used. For DSS-induced colitis, mice receive 3% DSS in drinking water ad libitum for 8 days. For TNBS-induced colitis, mice are anesthetized and treated with an intra-rectal injection of 3 rng TNBS in 20% ethanol (vol/vol) or vehicle alone as a control. For the chronic colitis model, all mice are treated with 3 cycles of 2% DSS for 5 days, followed by a 10-day recovery period. For all models, weight loss, stool consistency, and presence of fecal occult blood are monitored daily and used to calculate the disease activity index, as described (see, e.g., Correale C, 2013, Gastroenterology, February 2013, pp. 346-356.e3). Cohorts of mice are treated with antagonist anti-TREM2 antibodies every day starting from day 0 and subjected to DSS or TNBS administration. Mice are monitored every day, to check for weight loss, stool consistency, and presence of fecal occult blood were monitored daily and used to calculate the disease activity index, as described (see, e.g., S. Vetrano, (astroenterology, 135 (2008), pp. 173-184). In separate experiments, endoscopic and histological images of mucosal damage are collected to evaluate inflammatory cell infiltration and mucosal damage. Similar studies can be conducted to test the benefit of TREM2 antibodies in other models of autoimmunity including Crolin's disease, inflammatory bowel disease, and ulcerative colitis (see, e.g., Low et al., (2013) Drug Des Devel Ther.; 7: 1341-1357; and Said et al., (2008) PLoS Med 5(9): e198).

### Example 53: Analysis of the Protective Effect of Agonist TREM2 in Wound Healing

To evaluate the ability of anti-TREM2 antibodies to increase colonic wound repair following injury, a mouse model of biopsy injury in the colon is used. In this model, the endoscope with outer operating sheath is inserted to the mid-descending colon and the mucosa is surveyed to the ano-rectal junction. Then, a single full thickness area of the entire mucosa and submucosa is removed with flexible biopsy forceps with a diameter of 3 French, avoiding penetration of the muscularis propria. Each mouse is biopsy injured at 3-5 sites along the dorsal side of the colon (see. e.g., Seno H, 2008, Proc Natl Acad Sci USA. 2009 Jan. 6; 106(1): 256-261). Cohorts of mice are treated with agonist anti-TREM2 antibodies 2 or 3 days after biopsy injury. Mice are monitored every day for 15 days, to check for weight loss and wound healing by measuring the surface area of lesions.

### Example 54: Analysis of the Protective Effect of TREM2 Antibodies in Retinal Degeneration

AMD is a degenerative disease of the outer retina. It is thought that inflammation, particularly inflammatory cytokines and macrophages, contribute to AMD disease progression. The presence of macrophages in the proximity of AMD lesions is documented, in the drusen, Bruch's membrane, choroid and retina. Macrophages release tissue factor (TF) and vascular endothelial growth factor (VEGF), which triggers the expansion of new blood vessels formation in patients showing choroidal neovasulcarization. The type of macrophage present in the macular choroid changes with age, displaying elevated levels of M2 macrophages in older eyes compared to younger eyes. However, advanced AMD maculae had higher M1 to M2 rations compared to normal autopsied eyes of similar age. (see, e.g., Cao X et al, (2011), Pathol Int 61(9): pp 528-35). This suggests a link between classical M1 macrophage activation in the eye in the late onset of AMD progression. Retinal microglia cells are tissue-resident macrophages that are also normally present in the inner retina. In the event of damage, microglia can be activated and act as mediator of inflammation. Activated microglia has been detected in the AMD tissue samples and has been proposed as one potential contributor of inflammatory processed that lead to AMD pathogenesis (Gupta et al., (2003) Exp Eye Res., 76(4):463-71.). The ability of antagonist TREM2 antibodies to prevent, delay, or reverse AMD is tested in one or more of AMD models (see, e.g., Pennesi et al., (2012) Mol Aspects Med.; 33(4): 487-509). Overall inflammatory macrophages (either M1 and/or activated microglia) are documented to correlate with AMD disease progression and therefore represent a therapeutic target for antagonist TREM2 antibodies. Similar therapeutic benefit can be achieved in glaucoma and genetic forms or retinal degeneration such as retinitis pigmentosa.

The ability of TREM2 antibodies to prevent, delay, or reverse retinal ganglion cell degeneration in glaucoma is tested in a glaucoma model (see, e.g., El-Danaf et al., (2015). J Neurosci. 11; 35(6):2329-43). Likewise, the therapeutic benefit of REM2 in genetically induced retinal degeneration and retinitis pigmentosa is tested as described in Chang et al., (2002) Vision Res.; 42(4):517-25, and in “Retinal Degeneration Rat Model Resource Availability of P23H and S334ter Mutant Rhodopsin Transgenic Rats and RCS Inbred and RCS Congenic Strains of Rats,” MM LaVail, Jun. 30, 2011.

### Example 55: Analysis of the Protective Effect of TREM2 Antibodies in Adipogenesis and Diet-Induced Obesity

To test the effect of TREM2 antibodies in adipogenesis and obesity, a mouse model of high-fat diet (HFD) is used (see, e.g., Park et al., (2015) Diabetes. 64(1):117-27).

### Example 56: Analysis of the Protective Effect of TREM2 Antibodies in Malaria

TREM2 expression in the nonparenchymal liver cells closely correlates with resistance to liver stage infection with the malaria agent Plasmodium berghei (Gonsalves et al., (2013) Proc Natl Acad Sci 26; 110(48):19531-6). Without wishing to be bound to theory, it is believed that TREM2 antibodies increase resistence to liver stage infection with P. berghei. The ability of TREM2 antibodies to increase resistance to malaria infection is tested as described in Gonçalves et al., (2013) Proc Natl Acad Sci 26; 110(48):19531-6. Briefly, GFP-expressing P. berghei ANKA sporozoites are obtained by dissection of infected salivary glands from Anopheles stephensi mosquitoes. Sporozoite suspensions in RPMI medium are injected i.v. in 100 μL of inocula containing 10⁴ sporozoites per mouse. Livers are collected at 40 h after injection or survival, and parasitemia is followed for 28 days. For experimental cerebral malaria scoring, neurologic symptoms are monitored from day 5 after injection.

### Example 57: Analysis of the Protective Effect of TREM2 Antibodies in Osteoporosis

Bone is a dynamic organ constantly remodeled to support calcium homeostasis and structural needs. The osteoclast is the cell responsible for removing both the organic and inorganic components of bone. The osteoclast is derived from hematopoietic progenitors in the macrophage lineage and differentiates in response to the tumor necrosis factor family cytokine receptor activators of NFκB ligand. Osteoclasts, the only bone-resorbing cells, are central to the pathogenesis of osteoporosis and osteopetrosis (Novack et al., (2008) Annual Rev Pathol., 3:457-84). Osteoporosis is a progressive bone disease that is characterized by a decrease in bone mass and density which can lead to an increased risk of fracture. It is mostly manifested in the first years following menopause, when bone turnover is accelerated, with increased activity of both osteoclasts and osteoblasts. Owing to an imbalance in the processes of resorption and synthesis, however, the net effect is bone loss, which is largely trabecular. Thus, the most prevalent sites of fracture in osteoporosis are the wrist, femoral neck, and vertebral bodies, in which the trabecular structure is key to overall bone strength. Accelerated osteoclast differentiation and increased bone resorption capacity, resulting in osteoporosis have been described in animal models lacking the expression of TREM2 (Otero et al (2012) J. Immunol. 188, 2612-2621). Reduced osteoclast function results in osteopetrosis, with increased bone mass and elimination of bone marrow space, as observed in animal models lacking DAP12 ITAM signaling adapter and resulting in a significant defect in differentiation of osteoclast-like cells (Koga, et al., (2004) Nature 428: 758-763). Without wishing to be bound by theory, it is believed that administering an anti-TREM2 antibody of the present disclosure can prevent, reduce the risk of, and/or treat osteoporosis. In some embodiments, administering an agonist anti-TREM2 antibody may induce one or more TREM2 activities in an individual having osteopetrosis (e.g., DAP12 phosphorylation, Syk activation, and accelerated differentiation into osteoclasts) (Peng et al (2010). Sci Signal. 2010 18; 3 122; and Humphrey et al., (2006) J Bone Miner Res., 21(2):237-45).

### Example 58: Analysis of the Effect of Anti-TREM2 Antibodies in Mouse Models of Spinal Cord Injury

A total of 20 C57/BL6 mice were used in 2 groups of 10 mice each. Spinal cord injury (SCI) was induced according to Han et al., Brain 2010, 133:1026-42. Briefly, the mice were anaesthetized and their backs shaved and cleansed with Betadine (Purdue Products LP). After a midline incision and laminectomy of the T9 vertebra, spinal cord contusions were induced using the Infinite Horizon Impactor with the force set at 50 kilodyne (PSI, Lexington, Ky.). The vertebral column was stabilized in a frame with rigid steel clamps inserted under the transverse processes. Only mice with injury displacements of 400-800 □m were included. After the injury, the muscles were closed in layers, the skin incision was closed and Bacitracin zinc antibiotic (Altana, Melville, N.Y.) was applied on the incision area.

Animals were tested for locomotion until 6 weeks after injury. Anti-TREM2 antibody 7E5 and isotype control antibody (control IgG) were injected at 80 mg/kg once per week intraperitoneally, including one pre-injury injection 24 h prior to SCI induction.

To test motor skills, hindlimb performance in stepping, limb coordination, and trunk stability was tested on a 10-point scale, called the Basso Mouse Scale (Basso et al., J Neurotrauma 2006, 23:635-59). Basso Mouse Scale (BMS) scores of 0-2 involve hindlimb paralysis; 3-4 some ankle movement; 5-6 weight-bearing with some coordination and stepping; 7-9 high functioning with consistent coordinated stepping; and where 9 is normal. BMS subscores are calculated when mice have a score of 5 and over (weight bearing). These represent fine motor skills including frequency of plantar stepping, interlimb coordination, paw position during stepping, as well as trunk stability and tail positioning during locomotion. The subscore can reveal differences between groups when the BMS is the same. BMS was performed before and 24 hr after the first injection, and every week after that, again 24 hr after the weekly injections.

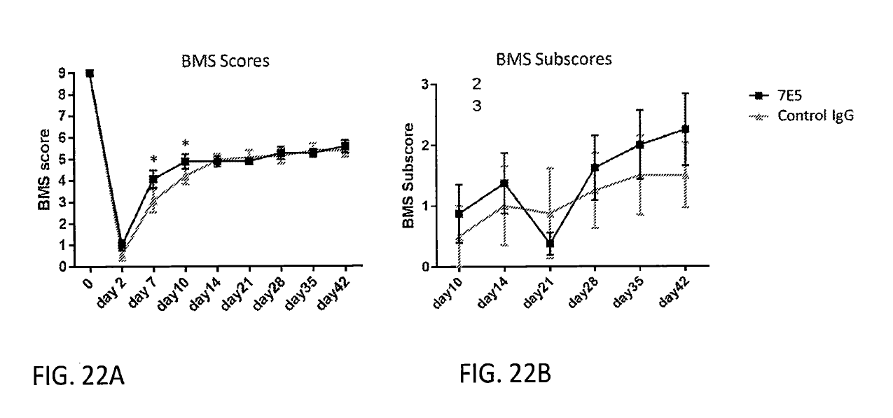

The results in FIGS. 22A and 22B show that treatment with the anti-TREM2 antibody 7E5 significantly, but transiently, improved BMS scores on days 7 and 10. The results may be due to a differential effect of microglial function on tissue recovery after injury.

### Example 59: Analysis of the Ability of TREM2 Antibodies to Affect Survival of Human Dendritic Cells In Vitro

Monocytes from peripheral blood mononuclear cells were isolated using RosetteSep™ human monocyte enrichment cocktail (Stem Cell Technoclogies) according to manufacturer's manual. Monocytes were cultured at 1×10⁶ cells/ml in complete RPMI media (10% FCS, Pen/Strep, L-Glutamine, MEM non-essential Amino Acid, Sodium Pyruvate, 1 mM HEPES) in the presence of 100 ng/ml hGM-CSF and 100 ng/ml hIL-4 for 7 days. Human dendritic cells were plated at 25,000 cells per well in a 96-well, non-tissue culture treated plate. Various concentrations of anti-TREM2 antibody 9F5 or 10 Kg/ml of anti-TREM2 antibody 10A9 was added in the presence of 20 ng/ml of hGM-CSF and 20 ng/ml of hIL-4. Mouse IgG1 isotype control antibody and media alone (no addition) were used as a control. After 3 days, cell viability was analyzed using a CellTiter-Glo® (Promega) according to manufacturer's protocol.

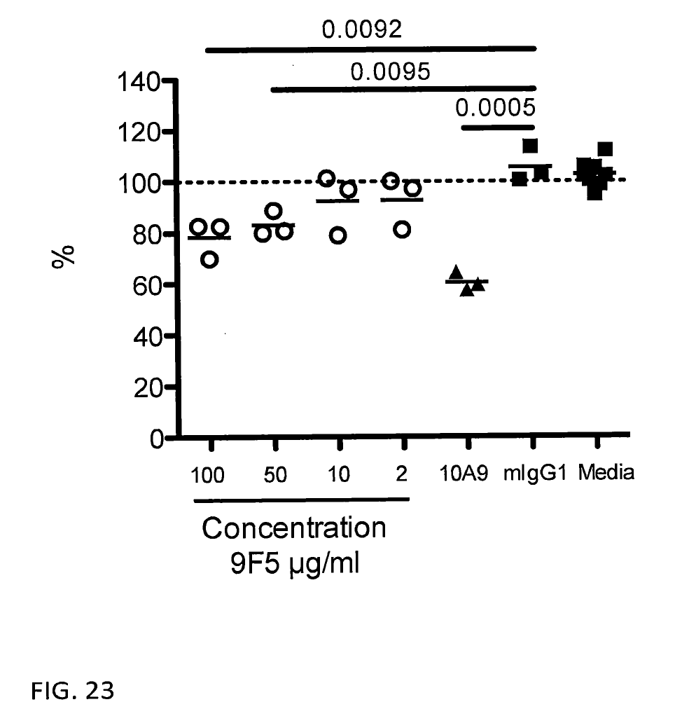

Human monocyte-derived dendritic cells were incubated for three days with either anti-TREM2 antibody 9F5 or 10A9. While incubation with 10 μg/ml of antibody 10A9 decreased cell survival to 60%, 10 μg/ml of antibody 9F5 did not significantly affect cell survival (FIG. 23).

### Example 60: TREM2 Antibodies Show Brain or CSF Levels at Above 1% of Peripheral Concentrations

Groups of wild-type (WT) or 5XFAD mice are chronically treated with anti-TREM2 antibody or control antibody for between four to twelve weeks by intraperitoneal weekly injection. Plasma is collected weekly. At the end of the study, mice are anesthetized with 3-5% isoflurane/oxygen mixture until unconscious and unresponsive to withdrawal reflex. The isoflurane/oxygen mixture is maintained throughout the procedure by delivery through a nose cone. Mice are placed on their back and an incision is made through the abdomen and diaphragm exposing the heart. The right atrium is cut to allow the blood to escape and 20-30 ml of sterile saline is injected by syringe and 25 gauge needle into the left ventricle. The saline is delivered at a slow, even pace until all of the blood had been removed and the liver appears blanched. The brain is then removed from the skull and placed in a sterile petri dish under a dissecting microscope. The cerebellum, midbrain, and hindbrain are removed and the brain is divided into two hemispheres by cutting through the midline. Both hippocampus and frontal cortex are collected and snap frozen. Brain lysates are prepared using ice cold N-Per (Thermo Fisher) with protease inhibitors according to manufacturer's instructions. Protein concentration in the lysates is measured using a BCA assay (Thermo Fisher). Antibody levels in blood plasma and brain lysates are measured using a custom IgG Meso Scale Discovery assay and IgG concentrations are used to measure percent brain concentration of antibodies.

### Example 61: TREM2 Antibodies Ameliorate Pathology in a Mouse Model of Chronic Colitis

### Materials and Methods

Seven week-old female C57BL/6 mice were subjected to the following dextran sodium sulfate (DSS) protocol. The experimenters were blinded and antibody solutions of either anti-TREM antibody 7E5 or control antibody MOPC-21 were intraperitoneally (IP) injected into the mice starting at day −3 before the first DSS cycle, and then twice per week for the entire experiment at a concentration of 40 mg/kg. After 3 days, mice were subjected to three oral cycles (7 days) of 1.5% DSS (molecular mass 40 kDa, MP Biomedicals, cat n° 160110, Lot n° Q1408), each followed by cycles of regular water (7 days). Severity of acute and chronic colitis was scored twice per week, using a disease activity index (DAI) score based on evaluation of body weight, diarrhea and presence of blood in stools. DAI was determined by scoring changes in: weight loss (0=none; 1=1 to 5%; 2 =5 to 10%; 3=10 to 20%; 4=>20%); stool consistency (0=normal; 2=loose; 4=diarrhea); and rectal bleeding (0=normal; 2=occult bleeding; 4=gross bleeding) to obtain a five grade (0-4) DAI.

Surviving mice were sacrificed at day 35, after blood serum collection and colon endoscopy. In addition, after measurement of colon length, half of the tissue was formalin-fixed and included in paraffin for histological evaluation.

The colon endoscopy gives the opportunity to evaluate the severity of colitis with different parameters, such as thickening of the mucosa, bleeding, and sometimes a granular mucosa surface, loss of blood vessel structure, and the presence of fibrin. Based on these endoscopic signs of inflammation, at the end of the experiment, inflammation was scored also by endoscopy with the following system: thickening of the colon (score 0=transparent, 1=moderate, 2=marked, 3=non transparent), changes of the vascular pattern (score 0=normal, 1=moderate, 2=marked, 3=bleeding), visible Fibrin (0=none, 1=little, 2=marked, 3=extreme), granularity of the mucosal surface (0=none, 1=moderate, 2=marked, 3=extreme). All sub-scores are summed up to obtain an overall colon score of 0-12.

### Results

The dextran sodium sulfate (DSS) experimental model is the most widely used mouse model of colitis. DSS is a water-soluble, negatively charged sulfated polysaccharide with a highly variable molecular weight ranging from 5 to 1400 kDa. The mechanism by which DSS induces intestinal inflammation is thought to be the result of damage to the epithelial monolayer lining the large intestine, allowing the dissemination of pro-inflammatory intestinal contents (e.g., bacteria and their products) into underlying tissue. The distal and rectal parts are the most affected segments of the colon after this experimental protocol.

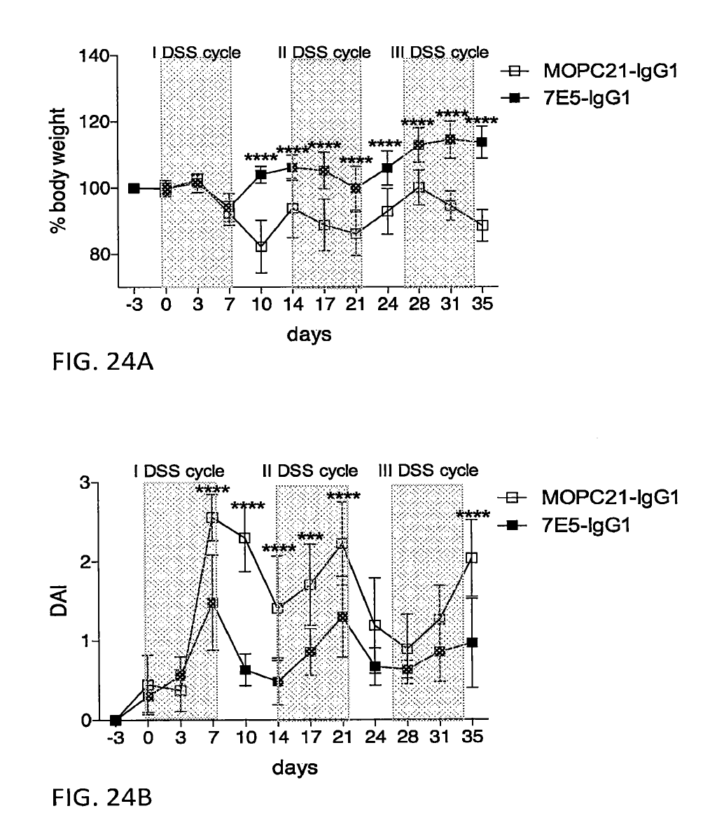

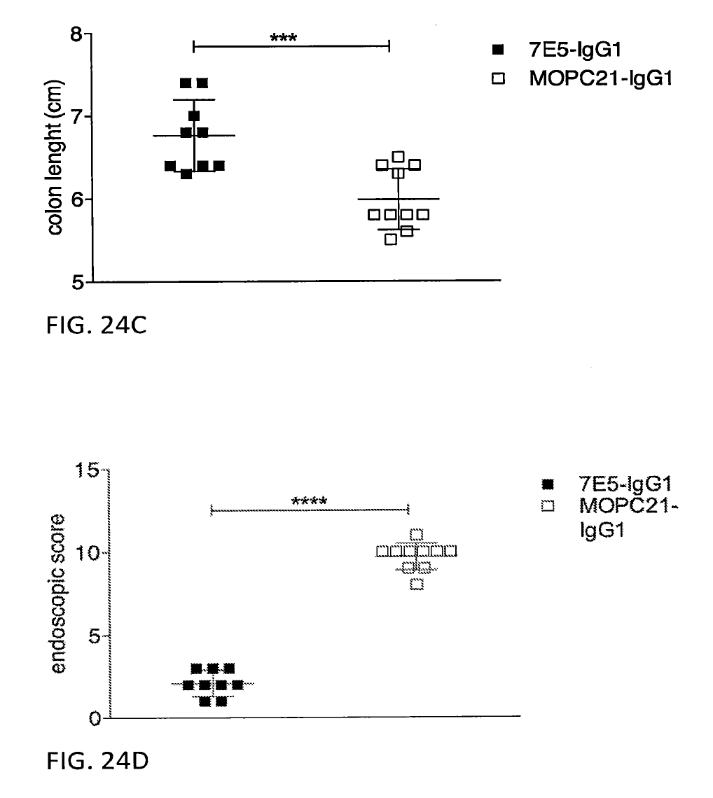

Mice treated with anti-TREM2 antibody 7E5 showed significantly reduced symptoms of inflammation after the first DSS cycle, as compared to mice treated with the control antibody. These results were also seen after the second and third DSS treatment (FIG. 24). Mice treated with anti-TREM2 antibody 7E5 showed a significant reduction in weight loss (FIG. 24A) and disease activity index (FIG. 24B) after the first DSS cycle and throughout the entire course of the study. At the end of the experiment, mice were sacrificed and the length of the colon was measured. Colon samples of mice injected with anti-TREM2 antibody 7E5 were significantly longer than colon samples of mice injected with the control antibody (FIG. 24C). This result further confirms that antibody 7E5 protects against DSS-induced colitis. Additionally, mice treated with anti-TREM2 antibody 7E5 showed a significantly lower endoscopic score, as compared to mice treated with the control antibody (FIG. 24D).

The results demostrate that treatment with an anti-TREM2 antibody significantly lowers symptoms of colitis in a chronic DSS mouse model. Moveover, these results indicate that anti-TREM2 antibodies, such as those of the present disclosure, could be used as a therapeutic for treting ulcerative colitis.

### Example 62: TREM2 Antibodies Show Activity in Humanized TREM2 Transgenic Mice

### Materials and Methods

To obtain mice expressing human TREM2 in the myeloid lineage, bacterial artificial chromosome (BAC) clones harboring TREM2, along with other TREM family members, and sufficient flanking sequences (at least 10 kb on either end) were identified. BAC clones harboring TREM gene loci were identified using the UCSC genome browser and the Clone DB at NCBI. Criteria were to identify clones with a minimum of 10 KB of flanking 5′ and 3′ sequences in addition to the genes of interest, to maximize the likelihood of appropriate gene expression.

BAC clones were obtained from Invitrogen as bacterial stab cultures. The cultures were grown and DNA was isolated using standard techniques. Agarose Gel electrophoresis after restriction digestion confirmed size and intactness of inserts, based on comparison with sequences of the UCSC genome browser. Additionally, sequences for BAC clones were queried at Clone DB at the NCBI web portal. which includes sequences for the ends of each BAC, as well as relevant human single nucleotide polymorphisms of interest.

Based on the above strategy, BAC clone CTD-3222A20 was identified. This clone contains the complete sequences of the human TREM2, human TREML2, human TREM1, human TREML1, and human TREML4 genes. As the TREM family of genes is found within a cluster on chromosome 6, a contiguous region covered by this BAC, from nucleotide 41104901-41292419, based on hg38 build of UCSC, spanning 187,519 nucleotides, included all of the genes.

Transgenic mice harboring BAC clone CTD-3222A20 were generated by injection of purified BAC DNA into C57BL6/j mouse zygotes utilizing standard pronuclear injection techniques. Zygotes were implanted into female mice. Pups from the implanted mice were then genotyped for the presence of the transgene. These founder mice were then bred to non-transgenic mice and the progeny were screened for appropriate expression of the transgenes. Briefly, blood was obtained from 4-8 week old mice, and monocytes were isolated using standard techniques. The monocytes were then analyzed by FACS using antibodies specific for each of the transgenes (i.e., TREM2, TREM1, and TREML2). Expression was confirmed for these genes.

For the experiments, either wild-type muce (WT) or humanize TREM2 BAC Trangenic mice (huTREM2 Tg or Bac-Tg) were intraperitoneally (IP) injected with 3% thioglycollate.

On day 4, peritoneal cells were collected from each mouse. In parallel, bone marrow was harvested to generate bone-marrow-derived macrophages according to standard procedures. To measure cytokine production upon stimulation of thioglycollate-induced macrophages, cells were incubated for about 60 hr on plates coated with either anti-TREM2 antibody 9F5 or control mouse antibody MOPC-21. TNF-alpha secreted in the supernatant was measured by cytometric bead array (CBA, BD Biosciences).

To examine expression of human vs. mouse TREM2 in WT vs. huTREM2 Tg mice, thioglycollate-induced macrophages were collected and stained with human TREM2-specific antibodies 9F5 or 10A9, with mouse TREM2-specific antibody 2F5, or with an anti-TREM2 antibody that recognizes both human and mouse TREM2 (R&D rat anti-TREM2) using standard cell staining protocols. Cells were analyzed with a FACS Canto and the live cell population was gated on CD11b+ and F4/80+. Raw data was analyzed by FlowJo.

For in vitro stimulation of bone marrow-derived macrophages from either WT or huTREM2 Tg mice, 10×10⁶ cells were either left unstimulated or stimulated with anti-TREM2 antibodu 9F5 or control antibody MOPC-21. Cells were then lysed and immunoprecipitated with rat anti-TREM2 antibody (R&D Systems). Samples were run on SDS-gels under non-reducing conditions and a Western immunoblot was performed with anti-phospho-tyrosine (Millipore) and anti-DAP12 antibodies (Cell signaling) using standard procedures.

### Results

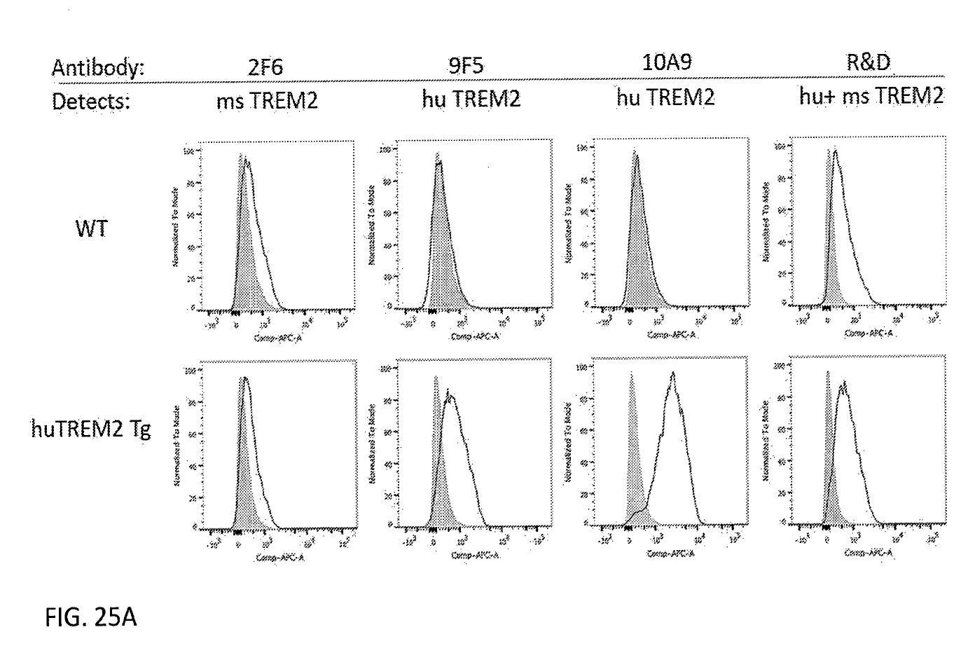

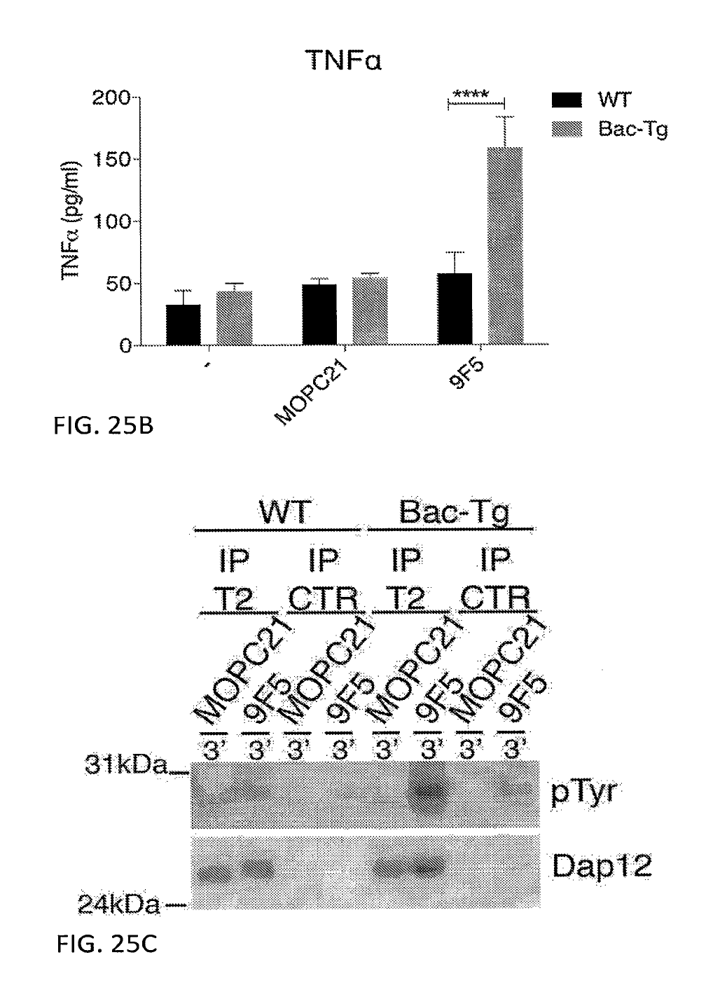

FIG. 25A shows that human TREM2 BAC transgenic mice express human TREM2, as there is positive TREM2 binding on thioglycollate-induced macrophages by both human specific anti-TREM2 antibodies 9F5 and 10A9, but these antibodies show no binding to WT macrophages that only express mouse TREM2.

Stimulating thioglycollate-induced macrophages from human TREM2 BAC transgenic mice in vitro with plate-bound anti-TREM2 antibody 9F5 for about 60 hr induced a significant increase in TNF-alpha secretion (FIG. 25B). In contrast, antibody 9F5 did not have an effect on thioglycollate-induced macrophages from WT mice.

Additionally, stimulating bone marrow-derived macrophages from human TREM2 BAC transgenic mice in vitro with anti-TREM2 antibody 9F5 induced Dap12 phosphorylation, while this effect was not observed with the control antibody (FIG. 25C).

The results in FIG. 25 indicate that anti-TREM2 antibody 9F5 can engage human TREM2 and induce TREM2-mediated signaling.

### Example 63: TREM2 Antibodies do not Bind Soluble TREM2 In Vivo

### Materials and Methods

Transgenic BAC mice expressing human TREM2 in the myeloid lineage were generated as described in Example 62.

Human TREM2 BAC transgenic mice (huTREM2 Tg) or wild-type mice (WT) were injected intraperitoneally with anti-TREM2 antibody 9F5, anti-TREM2 antibody T21-9, or isotype control mouse IgG1 antibody at 20 mg/kg (n=3 animals/group). Plasma samples were collected two days after the injection using standard procedures. Soluble TREM2 in plasma from either WT or TREM2 BAC transgenic mice was detected using a custom ELISA that specifically detects human TREM2. Briefly, 100 ul of 2 ug/ml human TREM2 specific capture antibody (8F11, msIgG) in PBS was incubated on high bind 96-well ELISA plates overnight at 4° C. The next day, plates were washed three times with 300 ul wash buffer (PBS+0.05% Tween) and 300 ul binding buffer (PBS+1% BSA) was added and incubated at room temperature (RT) for at least one hour. Plasma samples and standard (recombinant human TREM2-FC, R&D Systems) were diluted in binding buffer, added to the plate and incubated for 1 hour at RT. Then plates were washed again as before and biotinylated detection antibody (goat anti-human TREM2, R&D Systems) was added at 1:2000 dilution in binding buffer and incubated for 1h at RT. Plates were washed again and incubated with Streptavidin-HRP at 1:200 diluted in binding buffer for 20 min at RT. Plates were washed again and TMB substrate was added and incubated until color developed. The reaction was stopped upon addition of 2N sulfuric acid and plates were read on a BioTek Synergy™ H1 plate reader.

To test whether anti-TREM2 antibody 9F5 can bind to soluble TREM2 in plasma, a modified ELISA setup was tested. Plates were coated with 100 ul of a 2 ug/ml solution of 9F5, T21-9 or control IgG antibody in PBS overnight at 4° C. The next day, plates were washed three times with 300 ul wash buffer (PBS+0.05% Tween), and 300 ul binding buffer (PBS+1% BSA) was added and incubated at room temperature (RT) for at least one hour. Plasma samples from untreated TREM2 BAC transgenic mice were diluted in binding buffer, added to the plate and incubated for 1 hour at RT. Then plates were washed again as before and biotinylated detection antibody (goat anti-human TREM2, R&D Systems or rat anti-hu/ms TREM2, R&D Systems) was added at 1:2000 (for goat IgG) or 1:10,000 (for rat IgG) in binding buffer and incubated for 1 hr at RT. Plates were washed again and incubated with Streptavidin-HRP at 1:200 diluted in binding buffer for 20 min at RT. Plates were washed again and TMB substrate was added and incubated until color developed. The reaction was stopped upon addition of 2N sulfuric acid and plates were read on a BioTek Synergy™ H1 plate reader.

### Results

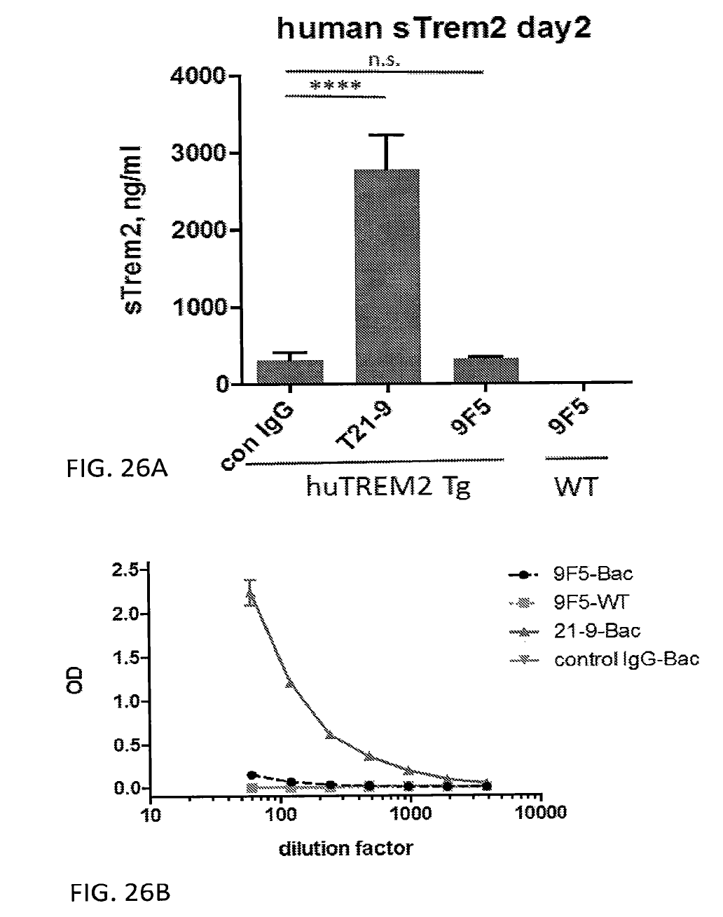

The results from the ELISA assay show that injection of T21-9 TREM2 antibody causes a highly significant increase in levels of soluble human TREM2 in plasma, while injection of antibody 9F5 does note increase levels of soluble human TREM2 in plasma (FIG. 26A). The ELISA could not detect any soluble TREM2 in WT mice that do not express human TREM2, confirming that the ELISA is specific for human TREM2 and does not recognize mouse TREM2. These results indicate that, in contrast to antibody T21-9, antibody 9F5 is not able to increase levels of soluble TREM2 in vivo.

It was hypothesized that antibody 9F5 may have failed to increase levels of soluble TREM2 in plasma because the antibody is not able to bind soluble TREM2. Antibody 9F5 was plated on ELISA plates to determine whether that antibody can capture endogenous soluble TREM2 from plasma. Again, in contrast to T21-9 which shows binding to solubleTREM2, antibody 9F5 only showed weak binding to soluble TREM2 (FIG. 26B). These results indicate that antibody 9F5 can only weakly bind to soluble TREM2 in plasma, which explains the inability of antibody 9F5 to increase soluble TREM2 in vivo.

In summary, the results indicate that antibody 9F5 cannot significantly bind to soluble TREM2 in vivo. This may be advantageous, as soluble TREM2 is thought to be an inactive version of the TREM2 receptor that can scavenge TREM2 antibodies, making them unable to bind to cellular TREM2, and thus lowering efficacy of TREM2 antibodies.

1. An isolated antibody that binds to a TREM2 protein, wherein the antibody induces one or more TREM2 activities and enhances one or more TREM2 activities induced by binding of one or more TREM2 ligands to the TREM2 protein.

2. The isolated antibody of claim 1, wherein the antibody enhances one or more TREM2 activities induced by binding of one or more TREM2 ligands to the TREM2 protein, as compared to the one or more TREM2 activities induced by binding of the one or more TREM2 ligands to the TREM2 protein in the absence of the isolated antibody.

3. The isolated antibody of claim 1, wherein the antibody enhances the one or more TREM2 activities without blocking binding of the one or more TREM2 ligands to the TREM2 protein.

4. The isolated antibody of claim 1, wherein the antibody does not compete with the one or more TREM2 ligands for binding to the TREM2 protein.

5. The isolated antibody of claim 1, wherein the antibody enhances binding of the one or more TREM2 ligands to the TREM2 protein.

6.-10. (canceled)

11. The isolated antibody of claim 1, wherein the antibody enhances the one or more TREM2 activities in the absence of cell surface clustering of TREM2.

12. The isolated antibody of claim 1, wherein the antibody enhances the one or more TREM2 activities by inducing or retaining cell surface clustering of TREM2.

13.-23. (canceled)

24. The isolated antibody of claim 1, wherein the TREM2 protein is a mammalian protein or a human protein.

25. The isolated antibody of claim 24, wherein the TREM2 protein is a wild-type protein, a naturally occurring variant, or a disease variant.

26.-27. (canceled)

28. The isolated antibody of claim 1, wherein the one or more TREM2 activities are selected from the group consisting of:
(a) TREM2 binding to DAP12;
(b) DAP12 phosphorylation;
(c) activation of Syk kinase;
(d) modulation of one or more pro-inflammatory mediators selected from the group consisting of IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells;
(e) recruitment of Syk to a DAP12/TREM2 complex;
(f) increasing activity of one or more TREM2-dependent genes, optionally wherein the one or more TREM2-dependent genes comprise nuclear factor of activated T-cells (NFAT) transcription factors;
(g) increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof;
(h) modulated expression of one or more stimulatory molecules selected from the group consisting of CD83, CD86 MHC class II, CD40, and any combination thereof, optionally wherein the CD40 is expressed on dendritic cells, monocytes, macrophages, or any combination thereof, and optionally wherein the dendritic cells comprise bone marrow-derived dendritic cells;
(i) increasing memory; and
(j) reducing cognitive deficit.

29. The isolated antibody of claim 1, wherein the antibody is of the IgG class, the IgM class, or the IgA class.

30. The isolated antibody of claim 29, wherein the antibody is of the IgG class and has an IgG1, IgG2, IgG3, or IgG4 isotype.

31.-36. (canceled)

37. The isolated antibody of claim 30, wherein:
(a) the isolated antibody has a human or mouse IgG1 isotype and comprises one or more amino acid substitutions in the Fc region at a residue position selected from the group consisting of: N297A, D265A, D270A, L234A, L235A, G237A, C226S, C229S, E233P, L234V, L234F, L235E, P331S, S267E, L328F, A330L, M252Y, S254T, T256E, L328E, P238D, S267E, L328F, E233D, G237D, H268D, P271G, A330R, and any combination thereof, wherein the numbering of the residues is according to EU numbering, or comprises an amino acid deletion in the Fc region at a position corresponding to glycine 236;
(b) the isolated antibody has an IgG1 isotype and comprises an IgG2 isotype heavy chain constant domain 1(CH1) and hinge region, optionally wherein the IgG2 isotype CH1 and hinge region comprises the amino acid sequence of ASTKGPSVFP LAPCSRSTSE STAALGCLVK DYFPEPVTVS WNSGALTSGVHTFPAVLQSS GLYSLSSVVT VPSSNFGTQT YTCNVDHKPS NTKVDKTVERKCCVECPPCP (SEQ ID NO: 886), and optionally wherein the antibody Fc region comprises a S267E amino acid substitution, a L328F amino acid substitution, or both, and/or a N297A or N297Q amino acid substitution, wherein the numbering of the residues is according to EU numbering;
(c) the isolated antibody has an IgG2 isotype and comprises one or more amino acid substitutions in the Fc region at a residue position selected from the group consisting of: P238S, V234A, G237A, H268A, H268Q, V309L, A330S, P331S, C214S, C232S, C233S, S267E, L328F, M252Y, S254T, T256E, H268E, N297A, N297Q, A330L, and any combination thereof, wherein the numbering of the residues is according to EU numbering;
(d) the isolated antibody has a human or mouse IgG4 isotype and comprises one or more amino acid substitutions in the Fc region at a residue position selected from the group consisting of: L235A, G237A, S228P, L236E, S267E, E318A, L328F, M252Y, S254T, T256E, E233P, F234V, L234A/F234A, S228P, S241P, L248E, T394D, N297A, N297Q, L235E, and any combination thereof, wherein the numbering of the residues is according to EU numbering; or
(e) the isolated antibody has a hybrid IgG2/4 isotype, and optionally wherein the antibody comprises an amino acid sequence comprising amino acids 118 to 260 of human IgG2 and amino acids 261 to 447 of human IgG4, wherein the numbering of the residues is according to EU numbering.

38.-47. (canceled)

48. The isolated antibody of claim 1, wherein the antibody binds to one or more amino acids within amino acid residues selected from the group consisting of:
i. amino acid residues 137-146 of SEQ ID NO: 1, or amino acid residues on a TREM2 protein corresponding to amino acid residues 137-146 of SEQ ID NO: 1;
ii. amino acid residues 139-147 of SEQ ID NO: 1, or amino acid residues on a TREM2 protein corresponding to amino acid residues 139-147 of SEQ ID NO: 1;
iii. amino acid residues 139-149 of SEQ ID NO: 1, or amino acid residues on a TREM2 protein corresponding to amino acid residues 139-149 of SEQ ID NO: 1;
iv. amino acid residues 142-152 of SEQ ID NO: 1, or amino acid residues on a TREM2 protein corresponding to amino acid residues 142-152 of SEQ ID NO: 1; and
v. amino acid residues 149-157 of SEQ ID NO: 1, or amino acid residues on a TREM2 protein corresponding to amino acid residues 149-157 of SEQ ID NO: 1.

49.-50. (canceled)

51. The isolated antibody of claim 48, wherein the antibody binds to one or more amino acid residues selected from the group consisting of E151, D152, H154, and E156 of SEQ ID NO: 1, or one or more amino acid residues on a mammalian TREM2 protein corresponding to an amino acid residue selected from the group consisting of E151, D152, H154, and E156 of SEQ ID NO: 1.

52. The isolated antibody of claim 1, wherein the antibody competes with one or more antibodies selected from the group consisting of 3B10, 8F8, 9F5, 9G3, 11A8, 12F9, 2F6, 3A7, 7E5, 11H5, and any combination thereof for binding to TREM2.

53.-54. (canceled)

55. The isolated antibody of claim 1, wherein:
(a) the HVR-L1 comprises the amino acid sequence of SEQ ID NO: 11 or an amino acid sequence with at least about 90% homology to the amino acid sequence of SEQ ID NO: 11, the HVR-L2 comprises the amino acid sequence of SEQ ID NO: 26 or an amino acid sequence with at least about 90% homology to the amino acid sequence of SEQ ID NO: 26, the HVR-L3 comprises the amino acid sequence of SEQ ID NO: 36 or an amino acid sequence with at least about 90% homology to the amino acid sequence of SEQ ID NO: 36, the HVR-H1 comprises the amino acid sequence of SEQ ID NO: 51 or an amino acid sequence with at least about 90% homology to the amino acid sequence of SEQ ID NO: 51, the HVR-H2 comprises the amino acid sequence of SEQ ID NO: 69 or an amino acid sequence with at least about 90% homology to the amino acid sequence of SEQ ID NO: 69, and the HVR-H3 comprises the amino acid sequence of SEQ ID NO: 88 or an amino acid sequence with at least about 90% homology to the amino acid sequence of SEQ ID NO: 88;
(b) the HVR-L1 comprises the amino acid sequence of SEQ ID NO: 14 or an amino acid sequence with at least about 90% homology to the amino acid sequence of SEQ ID NO: 14, the HVR-L2 comprises the amino acid sequence of SEQ ID NO: 28 or an amino acid sequence with at least about 90% homology to the amino acid sequence of SEQ ID NO: 28, the HVR-L3 comprises the amino acid sequence of SEQ ID NO: 39 or an amino acid sequence with at least about 90% homology to the amino acid sequence of SEQ ID NO: 39, the HVR-H1 comprises the amino acid sequence of SEQ ID NO: 53 or an amino acid sequence with at least about 90% homology to the amino acid sequence of SEQ ID NO: 53, the HVR-H2 comprises the amino acid sequence of SEQ ID NO: 71 or an amino acid sequence with at least about 90% homology to the amino acid sequence of SEQ ID NO: 71, and the HVR-H3 comprises the amino acid sequence of SEQ ID NO: 90 or an amino acid sequence with at least about 90% homology to the amino acid sequence of SEQ ID NO: 90;
(c) the HVR-L1 comprises the amino acid sequence of SEQ ID NO: 16 or an amino acid sequence with at least about 90% homology to the amino acid sequence of SEQ ID NO: 16, the HVR-L2 comprises the amino acid sequence of SEQ ID NO: 29 or an amino acid sequence with at least about 90% homology to the amino acid sequence of SEQ ID NO: 29, the HVR-L3 comprises the amino acid sequence of SEQ ID NO: 35 or an amino acid sequence with at least about 90% homology to the amino acid sequence of SEQ ID NO: 35, the HVR-H1 comprises the amino acid sequence of SEQ ID NO: 55 or an amino acid sequence with at least about 90% homology to the amino acid sequence of SEQ ID NO: 55, the HVR-H2 comprises the amino acid sequence of SEQ ID NO: 73 or an amino acid sequence with at least about 90% homology to the amino acid sequence of SEQ ID NO: 73, and the HVR-H3 comprises the amino acid sequence of SEQ ID NO: 92 or an amino acid sequence with at least about 90% homology to the amino acid sequence of SEQ ID NO: 92;
(d) the HVR-L1 comprises the amino acid sequence of SEQ ID NO: 19 or an amino acid sequence with at least about 90% homology to the amino acid sequence of SEQ ID NO: 19, the HVR-L2 comprises the amino acid sequence of SEQ ID NO: 28 or an amino acid sequence with at least about 90% homology to the amino acid sequence of SEQ ID NO: 28, the HVR-L3 comprises the amino acid sequence of SEQ ID NO: 43 or an amino acid sequence with at least about 90% homology to the amino acid sequence of SEQ ID NO: 43, the HVR-H1 comprises the amino acid sequence of SEQ ID NO: 60 or an amino acid sequence with at least about 90% homology to the amino acid sequence of SEQ ID NO: 60, the HVR-H2 comprises the amino acid sequence of SEQ ID NO: 78 or an amino acid sequence with at least about 90% homology to the amino acid sequence of SEQ ID NO: 78, and the HVR-H3 comprises the amino acid sequence of SEQ ID NO: 97 or an amino acid sequence with at least about 90% homology to the amino acid sequence of SEQ ID NO: 97;
(e) the HVR-L1 comprises the amino acid sequence of SEQ ID NO: 19 or an amino acid sequence with at least about 90% homology to the amino acid sequence of SEQ ID NO: 19, the HVR-L2 comprises the amino acid sequence of SEQ ID NO: 28 or an amino acid sequence with at least about 90% homology to the amino acid sequence of SEQ ID NO: 28, the HVR-L3 comprises the amino acid sequence of SEQ ID NO: 43 or an amino acid sequence with at least about 90% homology to the amino acid sequence of SEQ ID NO: 43, the HVR-H1 comprises the amino acid sequence of SEQ ID NO: 60 or an amino acid sequence with at least about 90% homology to the amino acid sequence of SEQ ID NO: 60, the HVR-H2 comprises the amino acid sequence of SEQ ID NO: 888 or an amino acid sequence with at least about 90% homology to the amino acid sequence of SEQ ID NO: 888, and the HVR-H3 comprises the amino acid sequence of SEQ ID NO: 97 or an amino acid sequence with at least about 90% homology to the amino acid sequence of SEQ ID NO: 97;
(f) the HVR-L1 comprises the amino acid sequence of SEQ ID NO: 21 or an amino acid sequence with at least about 90% homology to the amino acid sequence of SEQ ID NO: 21, the HVR-L2 comprises the amino acid sequence of SEQ ID NO: 32 or an amino acid sequence with at least about 90% homology to the amino acid sequence of SEQ ID NO:32, the HVR-L3 comprises the amino acid sequence of SEQ ID NO: 45 or an amino acid sequence with at least about 90% homology to the amino acid sequence of SEQ ID NO: 45, the HVR-H1 comprises the amino acid sequence of SEQ ID NO: 62 or an amino acid sequence with at least about 90% homology to the amino acid sequence of SEQ ID NO: 62, the HVR-H2 comprises the amino acid sequence of SEQ ID NO: 80 or an amino acid sequence with at least about 90% homology to the amino acid sequence of SEQ ID NO: 80, and the HVR-H3 comprises the amino acid sequence of SEQ ID NO: 99 or an amino acid sequence with at least about 90% homology to the amino acid sequence of SEQ ID NO: 99;
(g) the HVR-L1 comprises the amino acid sequence of SEQ ID NO: 22 or an amino acid sequence with at least about 90% homology to the amino acid sequence of SEQ ID NO: 22, the HVR-L2 comprises the amino acid sequence of SEQ ID NO: 29 or an amino acid sequence with at least about 90% homology to the amino acid sequence of SEQ ID NO: 29, the HVR-L3 comprises the amino acid sequence of SEQ ID NO: 46 or an amino acid sequence with at least about 90% homology to the amino acid sequence of SEQ ID NO: 46, the HVR-H1 comprises the amino acid sequence of SEQ ID NO: 63 or an amino acid sequence with at least about 90% homology to the amino acid sequence of SEQ ID NO: 63, the HVR-H2 comprises the amino acid sequence of SEQ ID NO: 82 or an amino acid sequence with at least about 90% homology to the amino acid sequence of SEQ ID NO: 82, and the HVR-H3 comprises the amino acid sequence of SEQ ID NO: 100 or an amino acid sequence with at least about 90% homology to the amino acid sequence of SEQ ID NO: 100; or
(h) the HVR-L1 comprises the amino acid sequence of SEQ ID NO: 16 or an amino acid sequence with at least about 90% homology to the amino acid sequence of SEQ ID NO: 16, the HVR-L2 comprises the amino acid sequence of SEQ ID NO: 29 or an amino acid sequence with at least about 90% homology to the amino acid sequence of SEQ ID NO: 29, the HVR-L3 comprises the amino acid sequence of SEQ ID NO: 35 or an amino acid sequence with at least about 90% homology to the amino acid sequence of SEQ ID NO: 35, the HVR-H1 comprises the amino acid sequence of SEQ ID NO: 65 or an amino acid sequence with at least about 90% homology to the amino acid sequence of SEQ ID NO: 65, the HVR-H2 comprises the amino acid sequence of SEQ ID NO: 84 or an amino acid sequence with at least about 90% homology to the amino acid sequence of SEQ ID NO: 84, and the HVR-H3 comprises the amino acid sequence of SEQ ID NO: 102 or an amino acid sequence with at least about 90% homology to the amino acid sequence of SEQ ID NO: 102.

56.-72. (canceled)

73. The isolated antibody of claim 25, wherein the antibody is a fragment and the fragment is a Fab, Fab′, Fab′-SH, F(ab′)2, Fv or scFv fragment.

74. The isolated antibody of claim 1, wherein the one or more TREM2 ligands are selected from the group consisting of E. coli cells, apoptotic cells, nucleic acids, anionic lipids, anionic lipids, APOE, APOE2, APOE3, APOE4, anionic APOE, anionic APOE2, anionic APOE3, anionic APOE4, lipidated APOE, lipidated APOE2, lipidated APOE3, lipidated APOE4, zwitterionic lipids, negatively charged phospholipids, phosphatidylserine, sulfatides, phosphatidylcholin, sphingomyelin, membrane phospholipids, lipidated proteins, proteolipids, lipidated peptides, lipidated amyloid beta peptide, and any combination thereof.

75. (canceled)

76. The isolated antibody of claim 1, wherein the antibody is a murine antibody, a humanized antibody, a bispecific antibody, a multivalent antibody, a conjugated antibody, or a chimeric antibody.

77. The isolated antibody of claim 1, wherein the antibody is a monoclonal antibody.

78.-81. (canceled)

82. The isolated antibody of claim 1, wherein the antibody binds specifically to both human TREM2 and mouse TREM2.

83. The isolated antibody of claim 1, wherein the antibody:
(a) has dissociation constant (K_{D}) for human TREM2 that ranges from about 12.8 nM to about 2.9 nM, or less than 2.9 nM, wherein the K_{D} is determined at a temperature of approximately 4° C.; or
(b) has dissociation constant (K_{D}) for mouse TREM2 that ranges from about 10.4 nM to about 1.2 nM, or less than 1.2 nM, wherein the K_{D} is determined at a temperature of approximately 4° C.

84.-91. (canceled)

92. An isolated nucleic acid comprising a nucleic acid sequence encoding the antibody of claim 1.

93. A vector comprising the nucleic acid of claim 92.

94. An isolated host cell comprising the vector of claim 93.

95. A method of producing an antibody that binds to TREM2, comprising culturing the cell of claim 94 so that the antibody is produced.

96. (canceled)

97. An isolated antibody that binds to TREM2 produced by the method of claim 95.

98. A pharmaceutical composition comprising the antibody of claim 1 and a pharmaceutically acceptable carrier.

99. A method of preventing, reducing risk, or treating an individual having a disease, disorder, or injury selected from the group consisting of dementia, frontotemporal dementia, Alzheimer's disease, Nasu-Hakola disease, cognitive deficit, memory loss, spinal cord injury, traumatic brain injury, multiple sclerosis, chronic colitis, ulcerative colitis, and cancer, comprising administering to an individual in need thereof a therapeutically effective amount of the isolated antibody of claim 1.

100.-101. (canceled)

102. The method of claim 99, wherein the disease, disorder, or injury is Alzheimer's disease.

103.-124. (canceled)

125. A method of inducing one or more TREM2 activities and enhancing one or more TREM2 activities induced by binding of one or more TREM2 ligands to a TREM2 protein in an individual in need thereof, comprising administering to the individual a therapeutically effective amount of the isolated antibody of claim 1.

126. A method of inducing or promoting innate immune cell survival in an individual in need thereof, comprising administering to the individual a therapeutically effective amount of the isolated antibody of claim 1.

127. (canceled)

128. The method of claim 99, wherein the individual has a heterozygous variant of TREM2, wherein the variant comprises one or more substitutions selected from the group consisting of:
i. a glutamic acid to stop codon substitution in the nucleic acid sequence encoding amino acid residue Glu14 of SEQ ID NO: 1;
ii. a glutamine to stop codon substitution in the nucleic acid sequence encoding amino acid residue Gln33 of SEQ ID NO: 1;
iii. a tryptophan to stop codon substitution in the nucleic acid sequence encoding amino acid residue Trp44 of SEQ ID NO: 1;
iv. an arginine to histidine amino acid substitution at an amino acid corresponding to amino acid residue Arg47 of SEQ ID NO: 1;
v. a tryptophan to stop codon substitution in the nucleic acid sequence encoding amino acid residue Trp78 of SEQ ID NO: 1;
vi. a valine to glycine amino acid substitution at an amino acid corresponding to amino acid residue Va1126 of SEQ ID NO: 1;
vii. an aspartic acid to glycine amino acid substitution at an amino acid corresponding to amino acid residue Asp134 of SEQ ID NO: 1; and
viii. a lysine to asparagine amino acid substitution at an amino acid corresponding to amino acid residue Lys186 of SEQ ID NO: 1.

129. The method of claim 99, wherein the individual has a heterozygous variant of TREM2, wherein the variant comprises a guanine nucleotide deletion at a nucleotide corresponding to nucleotide residue G313 of the nucleic acid sequence encoding SEQ ID NO: 1; a guanine nucleotide deletion at a nucleotide corresponding to nucleotide residue G267 of the nucleic acid sequence encoding SEQ ID NO: 1; or both.

130. The method of claim 99, wherein the individual has a heterozygous variant of DAP12, wherein the variant comprises one or more variants selected from the group consisting of:
i. a methionine to threonine substitution at an amino acid corresponding to amino acid residue Met1 of SEQ ID NO: 2;
ii. a glycine to arginine amino acid substitution at an amino acid corresponding to amino acid residue Gly49 of SEQ ID NO: 2;
iii. a deletion within exons 1-4 of the nucleic acid sequence encoding SEQ ID NO: 2;
iv. an insertion of 14 amino acid residues at exon 3 of the nucleic acid sequence encoding SEQ ID NO: 2; and
v. a guanine nucleotide deletion at a nucleotide corresponding to nucleotide residue G141 of the nucleic acid sequence encoding SEQ ID NO: 2.

131.-146. (canceled)
